(19) United States
(12) Patent Application Publication
(10) Pub. No.: US 2026/0317762 A1
(43) Pub. Date: Oct. 15, 2026

(54) Circular RNA Compositions and Methods

(71) Applicant: Orna Therapeutics, Inc., Watertown, MA (US)

(72) Inventors: Robert Alexander Wesselhoeft, Somerville, MA (US); Trent Stevens, Wakefield, MA (US); B. Nelson Chau, Needham, MA (US); Nikolay Rozhkov, Sharon, MA (US); Rahul Vungutur, Cambridge, MA (US)

(73) Assignee: Orna Therapeutics, Inc., Watertown, MA (US)

(21) Appl. No.: 19/481,366

(22) PCT Filed: May 3, 2024
(86) PCT No.: PCT/US2024/027627

§ 371 Date: Nov. 4, 2025

Related U.S. Application Data
(60) Provisional application No. 63/464,435, filed on May 5, 2023.
(60) Provisional application No. 63/470,064, filed on May 31, 2023.

(51) Int. Cl. C12N 15/113

(52) U.S. Cl. CPC C12N 15/113; C12N 2310/321; C12N 2310/322; C12N 2310/333; C12N 2310/334; C12N 2310/3341; C12N 2310/335; C12N 2310/336; C12N 2310/532

(57) ABSTRACT

Circular RNA and precursor RNA polynucleotides, along with related compositions and methods are described herein. In some embodiments, the circular RNA comprises, in the following order, a 3′ self-spliced exon segment, an intervening region, and a 5′ self-spliced exon segment. In some embodiments, the precursor RNA is a linear RNA comprising a 5′ combined accessory element comprising a 3′ permuted intron segment; an intervening region; and a 3′ combined accessory element comprising a 5′ permuted intron segment. In some embodiments, the circular RNA and/or precursor RNA polynucleotides comprise a coding region comprising an expression sequence. In some embodiments, the precursor RNA polynucleotide comprises a monotron. In some embodiments, the circular RNA has improved expression, functional stability, immunogenicity, ease of manufacturing, and/or half-life when compared to linear RNA. In some embodiments, the methods and constructs result in improved circularization efficiency and/or splicing efficiency as compared to a control RNA polynucleotide comprising a native intronic sequence. Also provided herein are precursor RNA polynucleotides, circular RNA polynucleotides, and DNA polynucleotides, including polynucleotides comprising at least one modified A, C, G, or U nucleotide or nucleoside. Also presented herein are methods and compositions for the manufacture and preparation of circularized RNAs, along with methods of administering said circular RNAs and related compositions to a subject in need thereof for treatment or prevention purposes.

### CROSS REFERENCE TO RELATED APPLICATIONS

This application claims the benefit of, and priority to, U.S. Provisional Application No. 63/464,435, filed on May 5, 2023; and U.S. Provisional Application No. 63/470,064, filed on May 31, 2023, the contents of each of which are hereby incorporated by reference in their entirety for all purposes.

### SEQUENCE LISTING

The present application contains a Sequence Listing which has been submitted electronically in XML format. Said XML copy, created on Apr. 19, 2024, is named “2024-04-19_01318-0006-OOPCT_SL.xml” and is 32,662,470 bytes in size. The information in the electronic format of the sequence listing is incorporated herein by reference in its entirety.

Conventional gene therapy involves the use of DNA for insertion of desired genetic information into host cells. The DNA introduced into the cell is usually integrated to a certain extent into the genome of one or more transfected cells, allowing for long-lasting action of the introduced genetic material in the host. While there may be substantial benefits to such sustained action, integration of exogenous DNA into a host genome may also have many deleterious effects. For example, it is possible that the introduced DNA will be inserted into an intact gene, resulting in a mutation which impedes or even totally eliminates the function of the endogenous gene. Thus, gene therapy with DNA may result in the impairment of a vital genetic function in the treated host, such as e.g., elimination or deleteriously reduced production of an essential enzyme or interruption of a gene critical for the regulation of cell growth, resulting in unregulated or cancerous cell proliferation. In addition, with conventional DNA based gene therapy it is necessary for effective expression of the desired gene product to include a strong promoter sequence, which again may lead to undesirable changes in the regulation of normal gene expression in the cell. It is also possible that the DNA based genetic material will result in the induction of undesired anti-DNA antibodies, which in turn, may trigger a possibly fatal immune response. Gene therapy approaches using viral vectors can also result in an adverse immune response. In some circumstances, the viral vector may even integrate into the host genome. In addition, production of clinical grade viral vectors is also expensive and time consuming. Targeting delivery of the introduced genetic material using viral vectors can also be difficult to control. Thus, while DNA based gene therapy has been evaluated for delivery of secreted proteins using viral vectors (U.S. Pat. No. 6,066,626; U.S. Publication No. US2004/0110709), these approaches may be limited for these various reasons.

In contrast to DNA, the use of RNA as a gene therapy agent is substantially safer because RNA does not involve the risk of being stably integrated into the genome of the transfected cell, thus eliminating the concern that the introduced genetic material will disrupt the normal functioning of an essential gene, or cause a mutation that results in deleterious or oncogenic effects, and extraneous promoter sequences are not required for effective translation of the encoded protein, again avoiding possible deleterious side effects. In addition, it is not necessary for RNA to enter the nucleus to perform its function, while DNA must overcome this major barrier.

Circular RNA (circRNA or oRNA®) is a stable form of RNA that provides an advantage compared to linear RNA in structure and function, especially in the case of molecules that are prone to folding in an inactive conformation (Wang and Ruffner, 1998). Circular RNA polynucleotides lack the free ends necessary for exonuclease-mediated degradation, causing them to be resistant to several mechanisms of RNA degradation and granting extended half-lives when compared to an equivalent linear RNA. Circularization may allow for the stabilization of RNA polynucleotides that generally suffer from short half-lives and may improve the overall efficacy of exogenous mRNA in a variety of applications. Circular RNA can also be particularly interesting and useful for in vivo applications, especially in the research area of RNA-based control of gene expression and therapeutics, including protein replacement therapy and vaccination.

Three main techniques for making circularized RNA in vitro are the splint-mediated method, the permuted intron-exon method, and the RNA ligase-mediated method. However, existing methodologies may be limited by the size of RNA that can be circularized, thus limiting their therapeutic application. The present disclosure addresses this need by providing methods and compositions for the manufacture and preparation of circularized RNAs via engineering of the sequences for the DNA template, precursor linear RNA, and ultimately the circular RNA, along with methods of manufacturing and preparing the circular RNA, and methods of treating a subject in need using said circular RNA.

In some embodiments, provided herein are circular RNA polynucleotides (also referred to herein as “circular RNA”) comprising, in the following order, a 3′ self-spliced exon segment, an intervening region, and a 5′ self-spliced exon segment. In some embodiments, the 3′ self-spliced exon segment and/or the 5′ self-spliced exon segment is selected from an exon segment disclosed herein, e.g., in Table A or Table B, or SEQ ID NOs: 2990-3668, 25573, and 25574.

In some embodiments, provided herein are circular RNA polynucleotides comprising, in the following order, i) a 5′ combined accessory element comprising a 3′ self-spliced exon segment; ii) an intervening region; and iii) a 3′ combined accessory element comprising a 5′ self-spliced exon segment. In some embodiments, the 3′ self-spliced exon segment and/or the 5′ self-spliced exon segment is selected from an exon segment disclosed herein, e.g., in Table A or Table B, or SEQ ID NOs: 2990-3668, 25573, and 25574.

In some embodiments, provided herein are circular RNA polynucleotides comprising, in the following order, i) a 5′ combined accessory element comprising a 3′ self-spliced exon segment, wherein the 3′ self-spliced exon segment comprises an exon segment; ii) an intervening region; and iii) a 3′ combined accessory element comprising a 5′ self-spliced exon segment, wherein the 5′ self-spliced exon segment comprises an exon segment. In some embodiments, the 3′ self-spliced exon segment and/or the 5′ self-spliced exon segment is selected from an exon segment disclosed herein, e.g., in Table A or Table B, or SEQ ID NOs: 2990-3668, 25573, and 25574.

In some embodiments, provided herein are circular RNA polynucleotides comprising, in the following order, i) a 5′ combined accessory element comprising a 3′ self-spliced exon segment, wherein the 3′ self-spliced exon segment comprises an exon segment and a 3′ nucleotide of a 3′ splice site dinucleotide; ii) an intervening region; and iii) a 3′ combined accessory element comprising a 5′ self-spliced exon segment, wherein the 5′ self-spliced exon segment comprises an exon segment and a 5′ nucleotide of a 5′ splice site dinucleotide. In some embodiments, the 3′ self-spliced exon segment and/or the 5′ self-spliced exon segment is selected from an exon segment disclosed herein, e.g., in Table A or Table B, or SEQ ID NOs: 2990-3668, 25573, and 25574.

In some embodiments, provided herein is a circular RNA polynucleotide comprising, in the following order, a 3′ self-spliced exon segment, an intervening region, and a 5′ self-spliced exon segment, wherein at least one of the 3′ or 5′ self-spliced exon segments is selected from an exon segment comprising a sequence selected from SEQ ID NOs: 2990-3668, 25573, and 25574.

In some embodiments, provided herein are precursor linear RNA polynucleotides that are capable of autocatalytically self-splicing and forming the circular RNA described herein, retaining the intervening region and a 3′ exon segment and a 5′ exon segment.

In some embodiments, provided herein are precursor linear RNA comprising, for example, a 5′ combined accessory element comprising a 3′ permuted intron segment; an intervening region; and a 3′ combined accessory element comprising a 5′ permuted intron segment. In some embodiments, provided herein are precursor linear RNA comprising, for example, a 5′ combined accessory element comprising a 3′ permuted intron segment and a 3′ permuted exon segment; an intervening region; and a 3′ combined accessory element comprising a 5′ permuted intron segment and a 5′ permuted exon segment. In some embodiments, provided herein are precursor linear RNA comprising a monotron, an intervening region, and a terminal sequence; or a terminal sequence, an intervening region, and a monotron. In some embodiments, provided herein are precursor linear RNA comprising at least one modified A, C, G, or U/T nucleotide or nucleoside. Provided herein are circular RNA produced from these precursors. In some embodiments, preparing circular RNA constructs based on the precursor RNA polynucleotides of the disclosure results in improved circularization efficiency and/or splicing efficiency as compared to a control RNA polynucleotide comprising a native intronic sequence.

### BRIEF DESCRIPTION OF THE DRAWINGS

FIG. 1A depicts a size exclusion-high-performance liquid chromatography (SEC-HPLC) analysis of a post-IVT reaction solution. Milli-absorbance units (mAU) were measured over the course of the retention time. The IVT reaction was performed on a DNA template comprising Anabaena intron and exon segments. The largest peak in the figure (beginning approximately at 9.25 min) provides the circular RNA collected post-IVT of the DNA template. The two peaks to the right of the largest peak (beginning approximately at 11.5 and 13 minutes) correspond to the spliced-out 5′ and 3′ intron fragments produced post-IVT of the DNA template. FIG. 1B illustrates a permuted intron construct design schematic used to develop the exemplary DNA templates of FIG. 1a. FIG. 1C shows the general placement of the splice site dinucleotides in the exemplary DNA templates. FIG. 1D provides an illustration of the splicing reaction of a linear precursor wherein the permutation occurs in a 5′ orientation created post IVT of the exemplary DNA template and resulting circular product. FIG. 1E provides an illustration of the splicing reaction of a linear precursor wherein the permutation occurs in a 3′ orientation created post IVT of the exemplary DNA template and resulting circular product.

FIG. 2 depicts fragment analyzer analysis of a post-IVT reaction solution. The IVT reaction was performed on a DNA template comprising Anabaena intron and exon segments. Relative fluorescent units (RFU) were measured for each of the peaks. The largest peak in the figure (2530 nucleotides) contains the circular RNA, precursor RNAs, or nicked circular RNAs produced post-IVT of the DNA template. The two peaks to the left of the largest peak (265 and 357) correspond to the spliced-out 5′ and 3′ intron fragments produced post-IVT of the DNA template. LM in the figure indicates the lowest marker.

FIG. 3 depicts estimated percent circulation measured for various post-IVT reaction solutions using a fragment analyzer (“FA”) or a SEC-HPLC (“SEC”). IntronPeak and circPeak in the figure correspond to whether the intron peaks (“IntronPeak”) or the circular RNA peaks (“circPeak”) in the fragment analyzer and/or SEC-HPLC results were used to measure the estimated percent circularization. Post-IVT reaction solutions were formed from IVT reactions on 12 different DNA templates comprising a 5′ and 3′ Anabaena intron segment.

FIG. 4A and FIG. 4B show intronic activity for permuted introns of several different species origins (e.g., from Azoarcus, Twort, Nostoc, Nodularia, S795, large subunit ribosomal RNA (LSU), Pleurocapsa, and/or Planktothrix). Depicted in FIG. 4A are SEC-HPLC chromatographs of post-IVT DNA template solutions comprising introns for the species.

FIG. 4B provides the circular to precursor RNA present post IVT fraction of each of the DNA templates used in FIG. 4a.

FIG. 5A and FIG. 5B depict percent estimated excised introns for Group I introns (FIG. 5A) and Group II introns (FIG. 5B) from various species origins. % estimated intron in the figures represent the percent of intronic fragments present post IVT of DNA templates comprising said Group I or Group II introns. Intron and exon sequences for FIGS. 5a and 5b are present in SEQ ID NOS: 2990-3130 (Group I) and 3131-3187 (Group II), respectively.

FIG. 6A, FIG. 6B, and FIG. 6C illustrate the permutation screening process used to preliminarily determine whether Group I or Group II intron generated maintained splicing activity post permutation. FIG. 6A represents the general intron screening process. FIG. 6B provides an example of 3 permutations—indicated by the arrows in the figure—that were made on a Hypocrea pallida sequence. FIG. 6C illustrates an exemplary linear RNA sequence (i.e., precursor RNA polynucleotide) schematic used to test the splicing activity of the permuted introns from FIG. 6A.

FIG. 7 shows percent circularization for 6 naturally occurring introns (e.g., Coxiella burnetti, Geosmithia argillacea, Agrobacterium tumefaciens, Hypocrea pallida, bulbithecium hyalosporum, and Myocarachis inversa) at three different permutation sites. Percent circularization was measured from the IVT reaction of a DNA template comprising the permuted introns. DNA templates in FIG. 7 comprise sequences from SEQ ID NOS: 2991-2993, 2997, 3054 and 2998.

FIG. 8A and FIG. 8B depict permutation heat maps of regions of an exemplary group I intron (e.g., Anabaena (FIG. 8A) and Azoarcus (FIG. 8B)). Each location in FIG. 8A corresponds with a permuted intron sequence from SEQ ID NOS: 3222-3483. Each number in each figure indicates a tested permutation site. The splice junctions are indicated by the triangles.

FIG. 9A provides percent circularization (e.g., estimated percent splicing) of Coxiella burnetti and Hypocrea pallida introns at 8 permutation sites. Percent circularization was measured from the IVT reaction of a DNA template comprising the permuted introns. FIG. 9B depicts the 8 permutation sites of Coxiella burnetti. FIG. 9C depicts the 8 permutation sites of Hypocrea pallida. In FIG. 9B and FIG. 9C, each of the numbers in the key to the left of the figure are represented by the arrows along the sequence and correspond to a specific permutation site.

FIG. 10A and FIG. 10B depict estimated percent splicing for RNA constructs with incrementally minimized exon segments. Estimated percent splicing for both FIGS. 10a and 10b was collected from constructs comprising Anabaena introns segments, exon segments, a CVB3 internal ribosome entry site (IRES), and firefly luciferase coding regions. FIG. 10A depicts results from constructs with 5′-terminal exon deletions from naturally occurring Anabaena 5′ exons. Original (51 nt) in FIG. 10a pertains to the RNA construct control comprising full-length 51 nucleotide (e.g., non-minimized) exon segments derived from SEQ ID NO: 3188. Constructs comprising minimized exon sequences used in FIG. 10a comprise a sequence from SEQ ID NOS: 3189-3197. FIG. 10B depicts results from constructs with 3′ terminal deletions from naturally occurring Anabaena 3′ exons. Original (15 nt) in FIG. 10a pertains to the RNA construct control comprising full-length 15 nucleotide (e.g., non-minimized) exon segments. Constructs comprising minimized exon sequences used in FIG. 10b comprise a sequence from SEQ ID NOS: 3198-3205. FIG. 10C illustrates the direction of deletion for the 3′ and/or 5′ exon segments (i.e., left arrow shows the incremental deletion from the 3′ end of the 3′ exon segment; the right arrow shows the incremental deletion from the 5′ end of the 5′ exon segment).

FIG. 11A and FIG. 11B depict estimated percent splicing for RNA constructs with incrementally minimized exon segments. Estimated percent splicing for FIG. 11A and FIG. 11B was collected from constructs comprising Anabaena (FIG. 11A) or Coxiella burnetti (FIG. 11B) introns segments and exon segments, a Caprine kobuvirus internal ribosome entry site (IRES), and firefly luciferase coding regions. FIG. 11A depicts results from constructs with 5′ or 3′-terminal exon deletions from a permuted Anabaena 5′ or 3′ exons. Constructs comprising minimized exon sequences in FIG. 11A comprise a sequence from SEQ ID NOS: 3579-3596. FIG. 11B depicts results from constructs with 5′ or 3′-terminal exon deletions from Coxiella burnetti 5′ or 3′ exons. Constructs comprising minimized exon sequences in FIG. 11B comprise a sequence from SEQ ID NOS: 3642-3664. 3′ exons (P1) were deleted from the 3′ terminal end of the 3′ exon; 5′ exons (P2) were deleted from the 5′ terminal end of the 5′ exon in both FIG. 11A and FIG. 11B. Std refers to a non-minimized pair of exons in both FIG. 11A and FIG. 11B.

FIG. 12A and FIG. 12B depict estimated percent splicing of permuted intron-exon (PIE) constructs with a single nucleotide swap within the splice junctions of either the 3′ intron and/or sequences from Anabaena (FIG. 12A) or Coxiella burnetii (FIG. 12B). PIE constructs were derived from naturally occurring Anabaena intron and exon sequences and comprise of SEQ ID NOS: 3572-3578. FIG. 12A depicts nucleotide swaps from natural intron and exon sequences. FIG. 12B depicts nucleotide swaps for spacer constructs and Coxiella burnetti exon sequences. PIE constructs were derived from naturally occurring Anabaena intron and exon sequences. DNA templates for FIG. 12B comprised of SEQ ID NOS: 3635-3641.

FIG. 13A and FIG. 13B depict percent estimated splicing for constructs comprising Anabaena (FIG. 13A) or Coxiella burnetti (FIG. 13B) permuted introns and exons with one or more nucleotide swaps in the exon segments. Nucleotide swaps were reverse complements and/or a random scramble of one or more nucleotides in either the 3′ exon (P1) or 5′ exon (P2). FIG. 13A constructs comprised SEQ ID NOS: 3622 and 3624-3627. FIG. 13B constructs comprised SEQ ID NOS: 3665-3668.

FIG. 14 provides a schematic of an intron deletion in one or more exemplary DNA templates. FIG. 14 depicts the deletion of 3′ intron segment including the naturally occurring nucleotide of the splice site dinucleotide. The internal and external accessory sequences indicated in the figure may comprise a spacer and/or homology arm.

FIG. 15 depicts estimated percent splicing of DNA templates with a deleted 3″ intron (P1) (e.g., DNA templates comprised in the following 5′ to 3′ order: a 3′ exon segment, an internal ribosome entry site (IRES), an expression sequence, a 5′ exon segment and a 5′ intron segment). A base pair of one of the splice junctions had also been swapped to one of the other three base pairs (e.g., indicated in the figure as initial nucleotide>swapped nucleotide, e.g., C to G). Intron and exon segments were derived from Anabaena DNA plasmids. IRESes were derived from CVB3 TRESes and the expression sequence encodes firefly luciferase. DNA templates were comprised of sequences in whole or in part from SEQ ID NOS: 3597-3603.

FIG. 16 depicts estimated percent splicing of constructs lacking a 3′ intron segment. DNA templates comprised a 3′ exon segment, an internal ribosome entry site (TRES), a firefly luciferase coding region, a 5′ exon segment and a 5′ intron segment. Each of the DNA templates also received exon minimization incrementally in the 5′ and or 3′ exon segments. DNA templates were comprised of sequences in whole or in part from SEQ ID NOS: 3597, and 3604-3621.

FIG. 17A, FIG. 17B, and FIG. 17C depict a construct comprising a 5′ terminal sequence and a 3′ monotron sequence. FIG. 17A illustrates an exemplary DNA template comprising the 5′ terminal sequence and 3′ monotron sequence along with the placement of the splice site nucleotides. FIG. 17B provides an illustration of splicing and circularization process of a linear precursor of the DNA template in FIG. 17A. FIG. 17C depicts a size exclusion-high-performance liquid chromatography (SEC-HPLC) analysis of a post-IVT reaction solution of the DNA template in FIG. 17A. Milli-absorbance units (mAU) were measured over the course of the retention time. The IVT reaction was performed on a DNA template comprising Anabaena intron and exon segments. The largest peak in the figure (beginning approximately at 10 min) provides the circular RNA collected post-IVT of the DNA template.

FIG. 18A, FIG. 18B, and FIG. 18C depicts a construct comprising a 3′ terminal sequence and a 5′ monotron sequence. FIG. 18A illustrates an exemplary DNA template comprising the 3′ terminal sequence and 5′ monotron sequence along with the placement of the splice site nucleotides. FIG. 18B provides an illustration of splicing and circularization process of a linear precursor of the DNA template in FIG. 18A. FIG. 18C depicts a size exclusion-high-performance liquid chromatography (SEC-HPLC) analysis of a post-IVT reaction solution of the DNA template in FIG. 18A. Milli-absorbance units (mAU) were measured over the course of the retention time. The IVT reaction was performed on a DNA template comprising Anabaena intron and exon segments. The largest peak in the figure (beginning approximately at 11.5 min) provides the circular RNA collected post-IVT of the DNA template.

FIG. 19A and FIG. 19B depict percent circular RNAs produced in RNA constructs developed with either Anabaena position 189 or position 230 permutation site in FIG. 8A. The constructs of the figures were designed to include accessory sequences (e.g., internal or external spacers and/or homology arms) or not include any accessory sequences (indicated by NA). Percent circular RNA produced was measured post SEC-HPLC analysis.

FIG. 20 provides percent splicing for initial DNA templates comprising accessory elements, including internal homology arms (IH), external homology arms (EH), internal spacers (IS), and/or external spacers (ES) of different lengths, as compared to a control lacking accessory elements. The DNA templates were comprised of sequences from SEQ ID NOS: 3484-3571.

FIG. 21 depicts estimated percent splicing for a construct with a 3′ monotron element (P2) lacking internal homology arms (IH). The standard construct (“Std”) comprises two permuted intron exon elements and no monotron or terminal elements. DNA template comprised a sequence from SEQ ID NO: 3628 or 3633-3634.

FIG. 22A and FIG. 22B depict estimated percent splicing for RNA constructs that were allowed to circularize co-transcriptionally and optionally allowed to refold (refolded constructs are indicated by “_R” in the figures). FIG. 22A illustrates results from two RNA constructs with different intron permutation sites. Each of the constructs in FIG. 22A were allowed to undergo co-transcription and the constructs indicated with “_R” were allowed an additional refold step. FIG. 22B shows results from RNA constructs with various 5′-terminal exon deletions (e.g., 10, 20, 40, 42, 44 nucleotide deletions). “Original” indicates the constructs containing non-minimized Anabaena exon structures with 51 nucleotides.

FIG. 23A provides a schematic showing the incorporation of m6A modifications in linear RNA constructs to form circular RNA constructs comprising m6A modifications. FIG. 23B depicts a gel of a post IVT reaction of various RNA samples. RNA samples comprised RNA comprising either 0%, 1%, 5%, 10% or 100% fed m6A modifications, a CBV3 internal ribosome entry site (IRES), firefly luciferase coding region, and Anabaena permuted intron-exon (PIE) segments.

FIG. 24A illustrates naturally occurring DNA plasmids comprising exon and Group I or Group II introns sequences used to form linear precursor RNA with selective modification regions for FIG. 24B. FIG. 24B, FIG. 24C, and FIG. 24D illustrate exemplary depiction of the two linear precursor RNA used to form a single construct with certain regions lacking modifications (e.g., introns). As shown in FIG. 24B, FIG. 24C, and FIG. 24D, one of the strands (top) comprises no modified nucleotides or nucleosides; the other of the two strands (bottom) comprises one or more modified nucleotides or nucleosides (indicated by the stars). FIG. 24B depicts two linear precursor RNAs, wherein each linear precursor RNA comprises two introns and two exon segments. FIG. 24C depicts two linear precursor RNAs, wherein each linear precursor RNA comprises a monotron intron and two exon segments. There are more than one variation of the reactions for the two linear precursor RNAs of FIG. 24C that may occur; the dotted and non-dotted reactions in steps 2 and 3 may occur simultaneously or independently of each other (e.g., the dotted reactions could occur first for steps 2 and 3 then be followed by the non-dotted reactions in steps 2 and 3 (not depicted in FIG. 24B) or the dotted and non-reaction of step 2 occurs at the same time and then is followed by the dotted and non-dotted reactions of step 3 (depicted in FIG. 24). FIG. 24D depicts two linear precursor RNAs, wherein one linear RNA precursor comprises an intron and two exon segments, while the other comprises a monotron intron and (non-monotron) intron segment along with two exons segments. In some embodiments, in each of FIG. 24B, FIG. 24C, and FIG. 24D, Strand 1 may have a transesterification reaction first. In alternative embodiments, Strand 2 may have a transesterification reaction first.

FIG. 25 depicts a size exclusion-high-performance liquid chromatography (SEC-HPLC) analysis of a post-IVT reaction solution of the DNA template comprising an intron developed from an Azoarcus position 11 permutation site in FIG. 8B. Milli-absorbance units (mAU) were measured over the course of the retention time. The IVT reaction was performed on a DNA template comprising Azoarcus intron and exon segments at low magnesium levels of 12.75 mM and treated either with or without exonuclease digestion. The arrows in the figure indicate the circular RNA and linear RNA collected post-IVT of the DNA template.

FIG. 26 provides percent circular RNAs produced in RNA constructs developed with either Anabaena position 230 permutation site (i.e., “L9a5” as labeled in FIG. 26) in FIG. 8A or Azoarcus position 11 permutation site in FIG. 8B. The constructs of the figures were designed to include accessory sequences (e.g., internal or external spacers and/or homology arms). Percent circular RNA produced was measured post SEC-HPLC analysis. Circular RNAs were produced from IVT reactions of DNA templates at either low magnesium levels (i.e., 12.75 mM) or standard reaction levels (i.e., 34 mM).

FIG. 27 depicts a permutation heat map of regions of an exemplary group I intron Tetrahyema. Each location in FIG. 27 corresponds with a permuted intron sequence from SEQ ID NO: 25573. Each number in each figure indicates a tested permutation site.

FIG. 28 depicts a permutation heat map of regions of an exemplary group I intron T4 td. Each location in FIG. 28 corresponds with a permuted intron sequence from SEQ ID NO: 25574. Each number in each figure indicates a tested permutation site.

FIG. 29 depicts a permutation heat map of regions of an exemplary group I intron Staphylococcus phage Twort. Each location in FIG. 29 corresponds with a permuted intron sequence from SEQ ID NO: 3006. Each number in each figure indicates a tested permutation site.

FIG. 30 depicts a permutation heat map of regions of an exemplary group I intron Coxiella Burnetii. Each location in FIG. 30 corresponds with a permuted intron sequence from SEQ ID NO: 2997. Each number in each figure indicates a tested permutation site.

FIG. 31A and FIG. 31B illustrate percent m6A modification incorporation (i.e., “% M6A Peak Area”) into IVT reactions of DNA templates comprising introns developed with either Anabaena position 230 permutation site in FIG. 8A (FIG. 31A) or Azoarcus position 12 permutation site in FIG. 8B (FIG. 31B). Amount of m6A modified nucleotide introduced into the IVT reaction was either at 0%, 1% 5%, 10%, or 50% (i.e., “% Fed M6A”).

FIG. 32 depicts estimated percent circular RNAs produced in RNA constructs developed with either m6A or m1Ψ modifications post IVT reaction of DNA template comprising a Caprine kobuvirus internal ribosome entry site (IRES) and Anabaena intron permuted at position of 230 in FIG. 8A. Percent circular RNA produced was measured post SEC-HPLC analysis. Amount of m6A or m1Ψ modified nucleotide introduced into the IVT reaction was either at 0%, 1% 5%, 10%, or 50% (i.e., “% Fed Base Modification”).

FIG. 33 depicts estimated percent circular RNAs produced in RNA constructs developed with either m6A or m1Ψ modifications post IVT reaction of DNA template comprising a Caprine kobuvirus internal ribosome entry site (IRES) and Anabaena intron permuted at position of 230 in FIG. 8A and subsequently purified using oligo-dT purification methods. Percent circular RNA produced was measured post SEC-HPLC analysis. Amount of m6A or m1Ψ modified nucleotide introduced into the IVT reaction was either at 0%, 1% 5%, 10%, or 50% (i.e., “% Fed Base Modification”).

FIG. 34A provides luminescence of circular RNAs encoding firefly luciferase in relative light units (“RLU”) (i.e., “Fluc Activity”) and percent circularization (i.e., “circ, %”) post IVT reaction of three DNA templates (e.g., “control”, “Anabaena” and “Azoarcus” as depicted in FIG. 34A) with transfection of either 0%, 1%, 5% or 10% m6A or m1Ψ base modifications. FIG. 34B provides IFNβ secretion levels of constructs provided in FIG. 34A. In FIGS. 34A-34B, all three DNA templates comprised: (1) a T7 polymerase promoter, (2) 5′ and 3′ permuted intron segments formed from the permuted site(s), (4) 5′ and 3′ exon segments, (5) internal ribosome entry site (IRES), (6) Fluc coding sequence and an XbaI restriction site. As depicted in FIG. 34A, “Control” DNA template comprises an intron developed with an Anabaena position 230 permutation site in FIG. 8A and a Caprine kobuvirus IRES; “Anabaena” DNA template comprises an intron developed with an Anabaena position 230 permutation site in FIG. 8A and a CoxsackievirusB3 (CVB3) IRES; and “Azoarcus” DNA template comprises an intron developed with an Azoarcus position 12 permutation site in FIG. 8B and a CVB3 IRES. “Mock” was a lipofectamine control. 5′ triphosphate hairpin RNA transfected cells (“3p-hpRNA”) was a positive control. “+RNAseR” indicates where RNase R or ribonuclease R was used to purify the circular RNA solutions.

FIG. 35A-35C depicts IFNβ secretion (FIG. 35A), IFNβ (FIG. 35B), IL-6 fold induction (FIG. 35C) of circular RNAs formed from DNA templates undergoing IVT reaction with either 0% or 5% m6A or m1Ψ base modifications. DNA templates comprised: (1) a T7 polymerase promoter, (2) 5′ and 3′ permuted intron segments formed from the permuted site(s), (4) 5′ and 3′ exon segments, (5) Caprine kobuvirus internal ribosome entry site (IRES), (6) Fluc coding sequence and an XbaI restriction site. “Mock” was a lipofectamine control. 5′ triphosphate hairpin RNA transfected cells (“3p-hpRNA”) was a positive control.

FIG. 36A depicts loss of circularization post IVT reaction of DNA templates with either 0%, 1%, 5%, 10%, and 50% m6A base modifications as compared to 0% m6A base modifications. DNA templates comprised introns of either Anabaena positions 8 or 230 permutation sites in FIG. 8A or Azoarcus positions 12 or 119 permutation sites in FIG. 8B. FIG. 36B depicts estimated percent circularization of constructs present in FIG. 36A as determined using SEC-HPLC.

FIG. 37 depicts a gel of a post IVT reaction of various RNA samples. RNA samples comprised RNA comprising a CBV3 internal ribosome entry site (IRES), firefly luciferase coding region, and Anabaena permuted intron-exon (PIE) segments. RNA samples undergone IVT with either 0%, 1%, 5%, 10% or 100% m6A modifications.

FIG. 38A depicts SEC-HPLC analysis of a two linear precursor (“Strand 1” and “Strand 2”). Strand 1 comprises a monotron sequence and a (non-monotron) intron segment. Strand 2 comprises two (non-monotron) intron segments. “dT+Exonuclease” are the circular RNA product formed from Strand 1 and Strand 2 after oligo-dT and exonuclease purification. FIG. 38B depicts the circular RNA product present in FIG. 38A after purification using oligo-dT only purification (“dT Purified”), and after purification using both oligo-dT and an exonuclease solution. FIG. 38C depicts an exonuclease control post IVT reaction of a DNA template capable of self-circularization and comprising Anabaena 5′ and 3′ intron segments.

FIG. 39A and FIG. 39B show circular RNA generated using in vitro transcription followed by purification reducing reactogenicity in a Balb/c mouse model.

FIG. 40A and FIG. 40B show circular RNA generated using in vitro transcription followed by purification reducing reactogenicity in a BLaER1 model.

### DETAILED DESCRIPTION

The present disclosure provides, among other things, precursor RNAs for producing circular RNAs and the produced circular RNAs. In some embodiments, such produced circular RNAs have improved properties, such as improved circularization efficiency. In some embodiments, the precursor RNAs comprise Group I or Group II exon and/or intron segments. In certain embodiments, the precursor RNAs and/or circular RNAs comprise one or more modified nucleotides or nucleosides. Also provided herein are related compositions (e.g., template DNAs or lipid nanoparticles). Also provided herein are methods for the selection, design, preparation, manufacture, formulation, and/or use of RNA preparations, such as precursor RNAs or circular RNAs.

Reference will now be made in detail to certain embodiments, examples of which are illustrated in the accompanying drawings. While the disclosure is described in conjunction with the illustrated embodiments, it will be understood that they are not intended to limit the disclosure to those embodiments. On the contrary, the disclosure is intended to cover all alternatives, modifications, and equivalents, which may be included within the disclosure as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### 1. Definitions

Unless stated otherwise, the following terms and phrases as used herein are intended to have the following meanings:

As used herein, linear nucleic acid molecules are said to have a “5′-terminus” (or “5′ end”) and a “3′-terminus” (or “3′ end”) because nucleic acid phosphodiester linkages occur at the 5′ carbon and 3′ carbon of the sugar moieties of the substituent mononucleotides. The end nucleotide of a polynucleotide at which a new linkage would be to a 5′ carbon is its 5′ terminal nucleotide. The end nucleotide of a polynucleotide at which a new linkage would be to a 3′ carbon is its 3′ terminal nucleotide. A “terminal nucleotide,” as used herein, is the nucleotide at the end position of the 3′- or 5′-terminus.

As used herein, the term “3′ intron segment” (or “3′ intron fragment”) refers to a sequence with at least 75%, at least 80%, at least 85%, at least 90%, at least 91%, at least 92%, at least 93%, at least 94%, at least 95%, at least 96%, at least 97%, at least 98%, at least 99%, or 100% similarity to the 3′-proximal end of a natural intron (e.g., a group I or group II intron). In certain embodiments, the 3′ intron segment includes the 5′ nucleotide of the splice site dinucleotide. “3′ exon segment” (or “3′ exon fragment”) refers to a sequence with at least 75%, at least 80%, at least 85%, at least 90%, at least 91%, at least 92%, at least 93%, at least 94%, at least 95%, at least 96%, at least 97%, at least 98%, at least 99%, or 100% similarity to the 5′-proximal end of an exon adjacent to a “3′ intron segment” as described herein. In certain embodiments, the 3′ exon segment includes the 3′ nucleotide of the splice site dinucleotide.

The term “5′ intron segment” (or “5′ intron fragment”) refers to a sequence with at least 75%, at least 80%, at least 85%, at least 90%, at least 91%, at least 92%, at least 93%, at least 94%, at least 95%, at least 96%, at least 97%, at least 98%, at least 99%, or higher 100% similarity to the 5′-proximal end of a natural intron (e.g., a group I or group II intron). In certain embodiments, the 5′ intron segment includes the 3′ nucleotide of the splice site dinucleotide. “5′ exon segment” (or “5′ exon fragment”) refers to a sequence with at least 75%, at least 80%, at least 85%, at least 90%, at least 91%, at least 92%, at least 93%, at least 94%, at least 95%, at least 96%, at least 97%, at least 98%, at least 99%, or higher 100% similarity to the 3′-proximal end of an exon adjacent to a “5′ intron segment” as described herein. In certain embodiments, the 5′ exon segment includes the 5′ nucleotide of the splice site dinucleotide.

In some embodiments, the 3′ intron segment and the 3′ exon segment together form a first portion of an autocatalytic or self-splicing intron-exon sequence. In some embodiments, the 5′ intron segment and the 5′ exon segment together form the remainder (i.e., second portion) of the autocatalytic or self-splicing intron-exon sequence. In these embodiments, a linear nucleic acid molecule, e.g., RNA, comprising the 3′ intron segment and the 3′ exon segment at the 5′ end of the linear nucleic acid molecule and further the 5′ intron segment and the 5′ exon segment at the 3′ end the linear nucleic acid molecule, is capable of autocatalytically self-splicing and thereby capable of forming a circular nucleic acid molecule, e.g., circular RNA. In these embodiments, the 3′ intron segment and the 5′ intron segments are excised from the circular nucleic acid molecule, e.g., circular RNA, and the 3′ exon segment and the 5′ exon segment are retained in the circular nucleic acid molecule, e.g., circular RNA. Each retained post-splicing exon segment may be referred to as a self-splicing or self-spliced exon segment, e.g., a 3′ self-splicing or self-spliced exon segment and a 5′ self-splicing or self-spliced exon segment.

In some embodiments, the intron segment is a “Group I intron” and the corresponding exon segment may be referred to as a “Group I exon” or “Group 1 self-splicing exon” or “Group I self-spliced exon segment” or the like. In some embodiments, the intron segment is a “Group II intron” and the corresponding exon segment may be referred to as a “Group II exon” or “Group II self-splicing exon” or “Group II self-spliced exon segment” or the like.

In some embodiments, the retained, post-splicing, self-splicing 3′ or 5′ exon segment is a non-coding sequence in the circular nucleic acid molecule, e.g., circular RNA. In some embodiments, the circular nucleic acid molecule, e.g., circular RNA, further comprises a desired coding sequence, and the retained, post-splicing, self-splicing 3′ or 5′ exon segment is (e.g., designed) to be a portion of the desired expression sequence, contiguous with the desired coding sequence, and/or in frame with the desired coding sequence.

Within a circular nucleic acid molecule, e.g., derived from a linear nucleic acid precursor, and comprising a coding sequence, the 5′ to 3′ orientation of the coding sequence may be used to inform whether other sequences within the circular nucleic acid are 5′ and/or 3′, e.g., for example, 5′ is nearer to the 5′ of the coding sequence, and the 3′ end is downstream of the coding sequence. As used herein, within a circular nucleic acid molecule, e.g., derived from a linear nucleic acid precursor, reference to a “5′” or “3′” portion of the molecule may correspond to the orientation of the sequence within the linear nucleic acid precursor.

As used herein, “splice site” refers to the junction consisting of a dinucleotide between an exon and an intron in an unspliced RNA. As used herein, the term “splice site” refers to a dinucleotide that is partially or fully included in a group I or group II intron and/or exon and between which a phosphodiester bond is cleaved during RNA circularization. A “splice site dinucleotide” refers two nucleotides: a 5′ splice site nucleotide and the 3′ splice site nucleotide. A “5′ splice site” refers to the natural 5′ dinucleotide of the intron and/or exon e.g., group I or group II intron and/or exon, while a “3′ splice site” refers to the natural 3′ dinucleotide of the intron and/or exon. Exemplary splice site dinucleotides are shown in the table below.

TABLE
Exemplary Splice Site Dinucleotides

| 5′ nt | 3′ nt | 5′ nt | 3′ nt | 5′ nt | 3′ nt | 5′ nt | 3′ nt | 5′ nt | 3′ nt | 5′ nt | 3′ nt | 5′ nt | 3′ nt | 5′ nt | 3′ nt |
|---|---|---|---|---|---|---|---|---|---|---|---|---|---|---|---|
| of a 3′ | of a 3′ | of a 5′ | of a 5′ | of a 3′ | of a 3′ | of a 5′ | of a 5′ | of a 3′ | of a 3′ | of a 5′ | of a 5′ | of a 3′ | of a 3′ | of a 5′ | of a 5′ |
| splice | splice | splice | splice | splice | splice | \|splice | splice | splice | splice | \|splice | splice | splice | splice | \|splice | splice |
| site | site | site | site | site | site | site | site | site | site | site | site | site | site | site | site |
| di-nt | di-nt | di-nt | di-nt | di-nt | di-nt | di-nt | di-nt | di-nt | di-nt | di-nt | di-nt | di-nt | di-nt | di-nt | di-nt |
| A | A | A | A | C | A | A | A | G | A | A | A | U | A | A | A |
| A | A | A | C | C | A | A | C | G | A | A | C | U | A | A | C |
| A | A | A | G | C | A | A | G | G | A | A | G | U | A | A | G |
| A | A | A | U | C | A | A | U | G | A | A | U | U | A | A | U |
| A | A | C | A | C | A | C | A | G | A | C | A | U | A | C | A |
| A | A | C | C | C | A | C | C | G | A | C | C | U | A | C | C |
| A | A | C | G | C | A | C | G | G | A | C | G | U | A | C | G |
| A | A | C | U | C | A | C | U | G | A | C | U | U | A | C | U |
| A | A | G | A | C | A | G | A | G | A | G | A | U | A | G | A |
| A | A | G | C | C | A | G | C | G | A | G | C | U | A | G | C |
| A | A | G | G | C | A | G | G | G | A | G | G | U | A | G | G |
| A | A | G | U | C | A | G | U | G | A | G | U | U | A | G | U |
| A | A | U | A | C | A | U | A | G | A | U | A | U | A | U | A |
| A | A | U | C | C | A | U | C | G | A | U | C | U | A | U | C |
| A | A | U | G | C | A | U | G | G | A | U | G | U | A | U | G |
| A | A | U | U | C | A | U | U | G | A | U | U | U | A | U | U |
| A | C | A | A | C | C | A | A | G | C | A | A | U | C | A | A |
| A | C | A | C | C | C | A | C | G | C | A | C | U | C | A | C |
| A | C | A | G | C | C | A | G | G | C | A | G | U | C | A | G |
| A | C | A | U | C | C | A | U | G | C | A | U | U | C | A | U |
| A | C | C | A | C | C | C | A | G | C | C | A | U | C | C | A |
| A | C | C | C | C | C | C | C | G | C | C | C | U | C | C | C |
| A | C | C | G | C | C | C | G | G | C | C | G | U | C | C | G |
| A | C | C | U | C | C | C | U | G | C | C | U | U | C | C | U |
| A | C | G | A | C | C | G | A | G | C | G | A | U | C | G | A |
| A | C | G | C | C | C | G | C | G | C | G | C | U | C | G | C |
| A | C | G | G | C | C | G | G | G | C | G | G | U | C | G | G |
| A | C | G | U | C | C | G | U | G | C | G | U | U | C | G | U |
| A | C | U | A | C | C | U | A | G | C | U | A | U | C | U | A |
| A | C | U | C | C | C | U | C | G | C | U | C | U | C | U | C |
| A | C | U | G | C | C | U | G | G | C | U | G | U | C | U | G |
| A | C | U | U | C | C | U | U | G | C | U | U | U | C | U | U |
| A | G | A | A | C | G | A | A | G | G | A | A | U | G | A | A |
| A | G | A | C | C | G | A | C | G | G | A | C | U | G | A | C |
| A | G | A | G | C | G | A | G | G | G | A | G | U | G | A | G |
| A | G | A | U | C | G | A | U | G | G | A | U | U | G | A | U |
| A | G | C | A | C | G | C | A | G | G | C | A | U | G | C | A |
| A | G | C | C | C | G | C | C | G | G | C | C | U | G | C | C |
| A | G | C | G | C | G | C | G | G | G | C | G | U | G | C | G |
| A | G | C | U | C | G | C | U | G | G | C | U | U | G | C | U |
| A | G | G | A | C | G | G | A | G | G | G | A | U | G | G | A |
| A | G | G | C | C | G | G | C | G | G | G | C | U | G | G | C |
| A | G | G | G | C | G | G | G | G | G | G | G | U | G | G | G |
| A | G | G | U | C | G | G | U | G | G | G | U | U | G | G | U |
| A | G | U | A | C | G | U | A | G | G | U | A | U | G | U | A |
| A | G | U | C | C | G | U | C | G | G | U | C | U | G | U | C |
| A | G | U | G | C | G | U | G | G | G | U | G | U | G | U | G |
| A | G | U | U | C | G | U | U | G | G | U | U | U | G | U | U |
| A | U | A | A | C | U | A | A | G | U | A | A | U | U | A | A |
| A | U | A | C | C | U | A | C | G | U | A | C | U | U | A | C |
| A | U | A | G | C | U | A | G | G | U | A | G | U | U | A | G |
| A | U | A | U | C | U | A | U | G | U | A | U | U | U | A | U |
| A | U | C | A | C | U | C | A | G | U | C | A | U | U | C | A |
| A | U | C | C | C | U | C | C | G | U | C | C | U | U | C | C |
| A | U | C | G | C | U | C | G | G | U | C | G | U | U | C | G |
| A | U | C | U | C | U | C | U | G | U | C | U | U | U | C | U |
| A | U | G | A | C | U | G | A | G | U | G | A | U | U | G | A |
| A | U | G | C | C | U | G | C | G | U | G | C | U | U | G | C |
| A | U | G | G | C | U | G | G | G | U | G | G | U | U | G | G |
| A | U | G | U | C | U | G | U | G | U | G | U | U | U | G | U |
| A | U | U | A | C | U | U | A | G | U | U | A | U | U | U | A |
| A | U | U | C | C | U | U | C | G | U | U | C | U | U | U | C |
| A | U | U | G | C | U | U | G | G | U | U | G | U | U | U | G |
| A | U | U | U | C | U | U | U | G | U | U | U | U | U | U | U |

As used herein, the term “permutation site” refers to a site in an intron and/or exon (e.g., a group I or II intron and/or exon) where a cut is made prior to permutation of the intron/or exon. For example, such a cut generates an intron sequence comprising a 3′ intron segment and a sequence comprising a 5′ intron segment (e.g., group I or group II intron fragments) that are permuted to be on either side of a stretch of precursor RNA to be circularized. The permuted intron segments are thereby called “3′ permuted intron segments” or “3′ permuted elements” and “5′ permuted intron segments” or “5′ permuted elements” in the context of said precursor RNA. As used herein, “permuted intron segment” and “permuted intron element” are used interchangeably. In some embodiments, the permutation site consists of a dinucleotide.

As used herein, the singular forms “a,” “an,” and “the” include plural referents unless the content clearly dictates otherwise. Thus, for example, reference to “a cell” includes combinations of two or more cells, or entire cultures of cells; reference to “a polynucleotide” includes, as a practical matter, many copies of that polynucleotide.

A used herein, the terms “about,” or “approximately” are understood as within a range of normal tolerance in the art, for example within 2 standard deviations of the mean. “About” can be understood as within 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01% of the stated value. Unless otherwise clear from the context, all numerical values provided herein are modified by the term “about.”

As used herein, “accessory element” or “accessory sequences” refers to internal spacer(s), external spacer(s), and/or homology arm(s). As used herein, a “combined accessory element” or “combined accessory sequences” comprises the accessory element and further comprises an intron and/or exon segment. In some embodiments, the accessory element increases circularization efficiency and/or translation efficiency in a circular RNA as compared to a control circular RNA without the accessory sequences.

As used herein, an “affinity sequence” or “affinity tag” is a region of a polynucleotide sequence ranging from one (1) nucleotide to hundreds or thousands of nucleotides containing a repeated set of nucleotides for the purposes of aiding purification of a polynucleotide sequence. For example, an affinity sequence may comprise, but is not limited to, a polyA or polyAC sequence. In some embodiments, affinity tags are used in purification methods, referred to herein as “affinity-purification,” in which selective binding of a binding agent to molecules comprising an affinity tag facilitates separation from molecules that do not comprise an affinity tag. In some embodiments, an affinity-purification method is a “negative selection” purification method, in which unwanted species, such as linear RNA, are selectively bound and removed and wanted species, such as circular RNA, are eluted and separated from unwanted species.

An “antigen” refers to any molecule that provokes an immune response or is capable of being bound by an antibody or an antigen binding molecule. The immune response may involve either antibody production, or the activation of specific immunologically—competent cells, or both. A person of skill in the art would readily understand that any macromolecule, including virtually all proteins or peptides, may serve as an antigen. An antigen may be endogenously expressed, i.e. expressed by genomic DNA, or may be recombinantly expressed. An antigen may be specific to a certain tissue, such as a cancer cell, or it may be broadly expressed. In addition, fragments of larger molecules may act as antigens. In some embodiments, antigens are tumor antigens.

An “antigen binding molecule,” “antigen binding portion,” or “antibody fragment” refers to any molecule that specifically binds to a desired antigen. In some embodiments, an antigen binding molecule comprises the antigen binding parts (e.g., CDRs) of an antibody or antibody-like molecule. An antigen binding molecule may include the antigenic complementarity determining regions (CDRs). Examples of antibody fragments include, but are not limited to, Fab, Fab′, F(ab′)2, Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In further embodiments, the antigen binding molecule is an antibody fragment, including one or more of the complementarity determining regions (CDRs) thereof, that specifically binds to the antigen. In further embodiments, the antigen binding molecule is a single chain variable fragment (scFv). In some embodiments, the antigen binding molecule comprises or consists of avimers.

The term “antibody” (Ab) includes, without limitation, a glycoprotein immunoglobulin which binds specifically to an antigen. In general, an antibody may comprise at least two heavy (H) chains and two light (L) chains interconnected by disulfide bonds, or an antigen-binding molecule thereof. Each H chain may comprise a heavy chain variable region (abbreviated herein as VH) and a heavy chain constant region. The heavy chain constant region can comprise three constant domains, CH1, CH2 and CH3. Each light chain can comprise a light chain variable region (abbreviated herein as VL) and a light chain constant region. The light chain constant region can comprise one constant domain, CL. The VH and VL regions may be further subdivided into regions of hypervariability, termed complementarity determining regions (CDRs), interspersed with regions that are more conserved, termed framework regions (FR). CDRs may be described by numbering known in the art, for example, Kabat numbering, Chothia numbering, AbM numbering, or contact numbering. Each VH and VL may comprise three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component of the classical complement system. Antibodies may include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain variable fragments (scFv), camelized antibodies, affybodies, Fab fragments, F(ab′)2 fragments, disulfide-linked variable fragments (sdFv), anti-idiotypic (anti-id) antibodies (including, e.g., anti-anti-Id antibodies), minibodies, domain antibodies, synthetic antibodies (sometimes referred to herein as “antibody mimetics”), and antigen-binding fragments of any of the above. In some embodiments, antibodies described herein refer to polyclonal antibody populations.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in humans. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some embodiments, the variable region is a human variable region. In some embodiments, the variable region comprises rodent or murine CDRs and human framework regions (FRs). In particular embodiments, the variable region is a primate (e.g., non-human primate) variable region. In some embodiments, the variable region comprises rodent or murine CDRs and primate (e.g., non-human primate) framework regions (FRs). The terms “VL” and “VL domain” are used interchangeably to refer to the light chain variable region of an antibody or an antigen-binding molecule thereof. The terms “VH” and “VH domain” are used interchangeably to refer to the heavy chain variable region of an antibody or an antigen-binding molecule thereof.

As used herein, the terms “constant region” and “constant domain” are interchangeable and have a meaning common in the art. The constant region is an antibody portion, e.g., a carboxyl terminal portion of a light and/or heavy chain which is not directly involved in binding of an antibody to antigen but which may exhibit various effector functions, such as interaction with the Fc receptor. The constant region of an immunoglobulin molecule generally has a more conserved amino acid sequence relative to an immunoglobulin variable domain.

As used herein, “aptamer” refers in general to either an oligonucleotide of a single defined sequence or a mixture of said nucleotides, wherein the mixture retains the properties of binding specifically to the target molecule (e.g., eukaryotic initiation factor, 40S ribosome, polyC binding protein, polyA binding protein, polypyrimidine tract-binding protein, argonaute protein family, Heterogeneous nuclear ribonucleoprotein K and La and related RNA-binding protein). Thus, as used herein “aptamer” denotes both singular and plural sequences of nucleotides, as defined hereinabove. The term “aptamer” is meant to refer to a single- or double-stranded nucleic acid which is capable of binding to a protein or other molecule. In general, aptamers preferably comprise about 10 to about 100 nucleotides, preferably about 15 to about 40 nucleotides, more preferably about 20 to about 40 nucleotides, in that oligonucleotides of a length that falls within these ranges are readily prepared by conventional techniques. Optionally, aptamers can further comprise a minimum of approximately 6 nucleotides, preferably 10, and more preferably 14 or 15 nucleotides, that are necessary to effect specific binding.

As used herein, “autoimmunity” is defined as persistent and progressive immune reactions to non-infectious self-antigens, as distinct from infectious non self-antigens from bacterial, viral, fungal, or parasitic organisms which invade and persist within mammals and humans. Autoimmune conditions include scleroderma, Grave's disease, Crohn's disease, Sjorgen's disease, multiple sclerosis, Hashimoto's disease, psoriasis, myasthenia gravis, autoimmune polyendocrinopathy syndromes, Type I diabetes mellitus (TIDM), autoimmune gastritis, autoimmune uveoretinitis, polymyositis, colitis, and thyroiditis, as well as in the generalized autoimmune diseases typified by human Lupus.

“Autoantigen” or “self-antigen” as used herein refers to an antigen or epitope which is native to the mammal and which is immunogenic in said mammal.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y may generally be represented by the dissociation constant (K_{D} or K_{d}). Affinity may be measured and/or expressed in a number of ways known in the art, including, but not limited to, equilibrium dissociation constant (K_{D}), and equilibrium association constant (K_{A} or Kₐ). The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} may be determined by techniques known to one of ordinary skill in the art, such as BIACORE® or KinExA.

As used herein, the term “specifically binds,” refers to molecules that bind to an antigen (e.g., epitope or immune complex) as such binding is understood by one skilled in the art. For example, a molecule that specifically binds to an antigen may bind to other peptides or polypeptides, generally with lower affinity as determined by, e.g., immunoassays, BIACORE®, KinExA 3000 instrument (Sapidyne Instruments, Boise, ID), or other assays known in the art. In a specific embodiment, molecules that specifically bind to an antigen bind to the antigen with a K_{A} that is at least 2 logs, 2.5 logs, 3 logs, 4 logs or greater than the K_{A} when the molecules bind to another antigen.

As used herein, “bicistronic RNA” refers to a polynucleotide that includes two expression sequences coding for two distinct proteins. These expression sequences can be separated by a nucleotide sequence encoding a cleavable peptide such as a protease cleavage site. They can also be separated by a ribosomal skipping element.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor. Examples of cancers that may be treated by the methods disclosed herein include, but are not limited to, cancers of the immune system including lymphoma, leukemia, myeloma, and other leukocyte malignancies. In some embodiments, the methods disclosed herein may be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, neoplasm of the central nervous system (CNS), primary CNS lymphoma, tumor angiogenesis, spinal axis tumor, brain stem glioma, pituitary adenoma, epidermoid cancer, squamous cell cancer, T cell lymphoma, environmentally induced cancers including those induced by asbestos, other B cell malignancies, and combinations of said cancers. In some embodiments, the methods disclosed herein may be used to reduce the tumor size of a tumor derived from, for example, sarcomas and carcinomas, fibrosarcoma, myxosarcoma, liposarcoma, chondrosarcoma, osteogenic sarcoma, Kaposi's sarcoma, sarcoma of soft tissue, and other sarcomas, synovioma, mesothelioma, Ewing's tumor, leiomyosarcoma, rhabdomyosarcoma, colon carcinoma, pancreatic cancer, breast cancer, ovarian cancer, prostate cancer, hepatocellular carcinomna, lung cancer, colorectal cancer, squamous cell carcinoma, basal cell carcinoma, adenocarcinoma (for example adenocarcinoma of the pancreas, colon, ovary, lung, breast, stomach, prostate, cervix, or esophagus), sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, carcinoma of the fallopian tubes, carcinoma of the endometrium, carcinoma of the cervix, carcinoma of the vagina, carcinoma of the vulva, carcinoma of the renal pelvis, CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma). The particular cancer may be responsive to chemo- or radiation therapy or the cancer may be refractory. A refractory cancer refers to a cancer that is not amenable to surgical intervention and the cancer is either initially unresponsive to chemo- or radiation therapy or the cancer becomes unresponsive over time.

As used herein, the terms “circRNA,” “circular polyribonucleotide,” “circular RNA,” “circularized RNA,” “circular RNA polynucleotide” and “oRNA” are used interchangeably and refer to a single-stranded polyribonucleotide wherein the 3′ and 5′ ends that are normally present in a linear RNA polynucleotide have been joined together, e.g., by covalent bonds. As used herein, such terms also include preparations comprising circRNAs.

As used herein, the term “circularization efficiency” refers to a measurement of the rate of formation of amount of resultant circular polyribonucleotide as compared to its linear starting material.

The expression sequences in the polynucleotide construct may be separated by a “cleavage site” sequence which enables polypeptides encoded by the expression sequences, once translated, to be expressed separately by the cell, e.g., eukaryotic cell. A “self-cleaving peptide” refers to a peptide which is translated without a peptide bond between two adjacent amino acids, or functions such that when the polypeptide comprising the proteins and the self-cleaving peptide is produced, it is immediately cleaved or separated into distinct and discrete first and second polypeptides without the need for any external cleavage activity.

As used herein, “co-administering” refers to administering a therapeutic agent provided herein in conjunction with one or more additional therapeutic agents sufficiently close in time such that the therapeutic agent provided herein can enhance the effect of the one or more additional therapeutic agents, or vice versa.

As used herein, “coding element,” “coding sequence,” “coding nucleic acid,” or “coding region” is region located within the expression sequence and encodings for one or more proteins or polypeptides (e.g., therapeutic protein).

As used herein, a “noncoding element,” “noncoding sequence,” “non-coding nucleic acid,” or “noncoding nucleic acid” is a region located within the expression sequence. This sequence by itself does not encode for a protein or polypeptide, but may have other regulatory functions, including but not limited, allow the overall polynucleotide to act as a biomarker or adjuvant to a specific cell.

A “costimulatory ligand,” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MIC) molecule loaded with peptide. A co-stimulatory ligand may include, but is not limited to, 3/TR6, 4-IBB ligand, agonist or antibody that binds Toll-like receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) LI. A co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD 18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD1-la, CDl-lb, CDl-lc, CDl-ld, CDS, CEACAM1, CRT AM, DAP-10, DNAM1 (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, IT GAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CD1 la/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Lyl08), SLAMF7, SLP-76, TNF, TNFr, TNFR2, Toll ligand receptor, TRANCE/RANKL, VLA1, or VLA-6, or fragments, truncations, or combinations thereof.

As used herein, an antigen binding molecule, an antibody, or an antigen binding molecule thereof “cross-competes” with a reference antibody or an antigen binding molecule thereof if the interaction between an antigen and the first binding molecule, an antibody, or an antigen binding molecule thereof blocks, limits, inhibits, or otherwise reduces the ability of the reference binding molecule, reference antibody, or an antigen binding molecule thereof to interact with the antigen. Cross competition may be complete, e.g., binding of the binding molecule to the antigen completely blocks the ability of the reference binding molecule to bind the antigen, or it may be partial, e.g., binding of the binding molecule to the antigen reduces the ability of the reference binding molecule to bind the antigen. In some embodiments, an antigen binding molecule that cross-competes with a reference antigen binding molecule binds the same or an overlapping epitope as the reference antigen binding molecule. In other embodiments, the antigen binding molecule that cross-competes with a reference antigen binding molecule binds a different epitope as the reference antigen binding molecule. Numerous types of competitive binding assays may be used to determine if one antigen binding molecule competes with another, for example: solid phase direct or indirect radioimmunoassay (RIA); solid phase direct or indirect enzyme immunoassay (EIA); sandwich competition assay (Stahli et al., 1983, Methods in Enzymology 9:242-253); solid phase direct biotin-avidin EIA (Kirkland et al., 1986, J. Immunol. 137:3614-3619); solid phase direct labeled assay, solid phase direct labeled sandwich assay (Harlow and Lane, 1988, Antibodies, A Laboratory Manual, Cold Spring Harbor Press); solid phase direct label RIA using 1-125 label (Morel et al., 1988, Molec. Immunol. 25:7-15); solid phase direct biotin-avidin EIA (Cheung, et al., 1990, Virology 176:546-552); and direct labeled RIA (Moldenhauer et al., 1990, Scand. J. Immunol. 32:77-82).

A “cytokine,” as used herein, refers to a non-antibody protein that is released by one cell in response to contact with a specific antigen, wherein the cytokine interacts with a second cell to mediate a response in the second cell. A cytokine may be endogenously expressed by a cell or administered to a subject. Cytokines may be released by immune cells, including macrophages, B cells, T cells, neutrophils, dendritic cells, eosinophils and mast cells to propagate an immune response. Cytokines may induce various responses in the recipient cell. Cytokines may include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines may promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-la, IL-lb, IL-6, IL-13, IL-17a, IL-23, IL-27, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), TGF-beta, IL-35, and perforin. Examples of acute phase-proteins include, but are not limited to, C-reactive protein (CRP) and serum amyloid A (SAA).

The terms “deoxyribonucleic acid” and “DNA” as used herein mean a polymer composed of deoxyribonucleotides. The terms “ribonucleic acid” and “RNA” as used herein mean a polymer composed of ribonucleotides.

As used herein, the term “DNA template” refers to a DNA sequence capable of transcribing a linear RNA polynucleotide. For example, but not intending to be limiting, a DNA template may include a DNA vector, PCR product or plasmid.

As used herein, the terms “duplexed,” “double-stranded,” and “hybridized” are used interchangeably and refer to double-stranded nucleic acids formed by hybridization of two single strands of nucleic acids containing complementary sequences. Sequences of the two single-stranded nucleic acids can be fully complementary or partially complementary. In some embodiments, a nucleic acid provided herein may be fully double-stranded or partially double-stranded. In most cases, genomic DNA is double-stranded.

As used herein, two “duplex sequences,” “duplex forming sequences,” “duplex region,” “duplex forming regions,” “homology arms,” or “homology regions,” complement, or are complementary, fully or partially, to one another when the two regions share a sufficient level of sequence identity to one another's reverse complement to act as substrates for a hybridization reaction. In some embodiments, two duplex forming sequences are thermodynamically favored to cross-pair in a sequence specific interaction. As used herein, polynucleotide sequences have “homology” when they are either identical or share sequence identity to a reverse complement or “complementary” sequence. The percent sequence identity between a homology region and a counterpart homology region's reverse complement can be any percent of sequence identity that allows for hybridization to occur. In some embodiments, an internal duplex forming region of a polynucleotide disclosed herein is capable of forming a duplex with another internal duplex forming region and does not form a duplex with an external duplex forming region.

As used herein, the term “encode” refers broadly to any process whereby the information in a polymeric macromolecule is used to direct the production of a second molecule that is different from the first. The second molecule may have a chemical structure that is different from the chemical nature of the first molecule. For example, a DNA template (e.g., a DNA vector) may encode a RNA polynucleotide; a precursor RNA polynucleotide (e.g., a linear precursor RNA polynucleotide) may encode a mature RNA polynucleotide (e.g., a circular RNA polynucleotide).

As used herein, “endogenous” means a substance that is native to, i.e., naturally originated from, a biological system (e.g., an organism, a tissue, or a cell). For example, in some embodiments, a “endogenous polynucleotide” is normally expressed in a cell or tissue. In some embodiments, a polynucleotide is still considered endogenous if the control sequences, such as a promoter or enhancer sequences which activate transcription or translation, have been altered through recombinant techniques.

As used herein, the term “heterologous” means from any source other than naturally occurring sequences.

As used herein, an “endonuclease site” refers to a stretch of nucleotides within a polynucleotide that is capable of being recognized and cleaved by an endonuclease protein.

An “eukaryotic initiation factor” or “eIF” refers to a protein or protein complex used in assembling an initiator tRNA, 40S and 60S ribosomal subunits required for initiating eukaryotic translation.

As used herein, an “epitope” is a term in the art and refers to a localized region of an antigen to which an antibody may specifically bind. An epitope may be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some embodiments, the epitope to which an antibody binds may be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giege R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody: antigen crystals may be studied using well known X-ray diffraction techniques and may be refined using computer software such as X-PLOR (Yale University, 1992, distributed by Molecular Simulations, Inc.; see e.g. Meth Enzymol (1985) volumes 114 & 115, eds Wyckoff H W et al.; U.S. Patent Publication No. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323).

As used herein, the term “expression sequence” refers to a nucleic acid sequence that encodes a product, e.g., a peptide or polypeptide, regulatory nucleic acid, or non-coding nucleic acid. An exemplary expression sequence that codes for a peptide or polypeptide can comprise a plurality of nucleotide triads, each of which can code for an amino acid and is termed as a “codon.”

As used herein, a “fusion protein” is a protein with at least two domains that are encoded by separate genes that have been joined to transcribe for a single peptide.

As used herein, the term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

As used herein, an “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

As used herein, the term “immunogenic” or “immunostimulatory” refers to a potential to induce an immune response to a substance. An immune response may be induced when an immune system of an organism or a certain type of immune cells is exposed to an immunogenic substance. The term “non-immunogenic” refers to a lack of or absence of an immune response above a detectable threshold to a substance. No immune response is detected when an immune system of an organism or a certain type of immune cells is exposed to a non-immunogenic substance. In some embodiments, a non-immunogenic circular polyribonucleotide as provided herein, does not induce an immune response above a pre-determined threshold when measured by an immunogenicity assay. In some embodiments, no innate immune response is detected when an immune system of an organism or a certain type of immune cells is exposed to a non-immunogenic circular polyribonucleotide as provided herein. In some embodiments, no adaptive immune response is detected when an immune system of an organism or a certain type of immune cell is exposed to a non-immunogenic circular polyribonucleotide as provided herein.

As used herein, an “internal ribosome entry site” or “IRES” refers to an RNA sequence or structural element ranging in size from 10 nt to 1000 nt or more, capable of initiating translation of a polypeptide in the absence of a typical RNA cap structure. An exemplary IRES can be about 500 nt to about 700 nt in length.

As used herein, an “intervening region” refers to the portion of an RNA sequence that comprises one or more noncoding or one or more coding elements, or combinations thereof (e.g., translation initiation element, coding element, and/or stop codon) between splice sites. In some embodiments, the intervening regions are between the 5′ combined accessory element and the 3′ combined accessory element or between the 3′ intron fragment and the 5′ intron fragment in a precursor RNA polynucleotide. In some embodiments, the intervening region is between the monotron element and terminal element in other precursor RNA polynucleotides.

As used herein, “isolated” or “purified” generally refers to isolation of a substance (for example, in some embodiments, a compound, a polynucleotide, a protein, a polypeptide, a polynucleotide composition, or a polypeptide composition) such that the substance comprises a significant percent (e.g., greater than 1%, greater than 2%, greater than 5%, greater than 10%, greater than 20%, greater than 50%, or more, usually up to about 90%-100%) of the sample in which it resides. In certain embodiments, a substantially purified component comprises at least 50, 60, 70, 75, 80, 85, 90, 95, 96, 97, 98, or 99% of the sample. In additional embodiments, a substantially purified component comprises about, 80%-85%, or 90%-95%, 95-99%, 96-99%, 97-99%, or 95-100% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is more than as it is found naturally.

As used herein, a “leading untranslated sequence” is a region of polynucleotide sequences ranging from 1 nucleotide to hundreds of nucleotides located at the upmost 5′ end of a polynucleotide sequence. The sequences can be defined or can be random. A leading untranslated sequence is non-coding.

As used herein, a “terminal untranslated sequence” is a region of polynucleotide sequences ranging from 1 nucleotide to hundreds of nucleotides located at the downmost 3′ end of a polynucleotide sequence. The sequences can be defined or can be random. A terminal untranslated sequence is non-coding.

As used herein, the terms “terminal sequence” or “terminal element” are used interchangeably to refer to an RNA sequence capable of complexing with a monotron sequence or monotron element. The terminal sequence comprises a splice site nucleotide from the natural group I or group II intron present in the monotron. In some embodiments, the terminal sequence further comprises a natural exon or a fragment thereof and/or a synthetic sequence.

The term “lymphocyte” as used herein includes natural killer (NK) cells, T cells, or B cells. NK cells are a type of cytotoxic (cell toxic) lymphocyte that represent a major component of the innate immune system. NK cells reject tumors and cells infected by viruses. It works through the process of apoptosis or programmed cell death. They were termed “natural killers” because they do not require activation in order to kill cells. T cells play a major role in cell-mediated-immunity (no antibody involvement). T cell receptors (TCR) differentiate T cells from other lymphocyte types. The thymus, a specialized organ of the immune system, is the primary site for T cell maturation. There are numerous types of T cells, including: helper T cells (e.g., CD4+ cells), cytotoxic T cells (also known as TC, cytotoxic T lymphocytes, CTL, T-killer cells, cytolytic T cells, CD8+ T cells or killer T cells), memory T cells ((i) stem memory cells (TSCM), like naive cells, are CD45RO−, CCR7+, CD45RA+, CD62L+ (L-selectin), CD27+, CD28+ and IL-7Ra+, but also express large amounts of CD95, IL-2R, CXCR3, and LFA-1, and show numerous functional attributes distinctive of memory cells); (ii) central memory cells (TCM) express L-selectin and CCR7, they secrete IL-2, but not IFNγ or IL-4, and (iii) effector memory cells (TEM), however, do not express L-selectin or CCR7 but produce effector cytokines like IFNγ and IL-4), regulatory T cells (Tregs, suppressor T cells, or CD4+CD25+ or CD4+ FoxP3+ regulatory T cells), natural killer T cells (NKT) and gamma delta T cells. B-cells, on the other hand, play a principal role in humoral immunity (with antibody involvement). B-cells make antibodies, are capable of acting as antigen-presenting cells (APCs) and turn into memory B-cells and plasma cells, both short-lived and long-lived, after activation by antigen interaction. In mammals, immature B-cells are formed in the bone marrow.

As used herein, a “miRNA site” refers to a stretch of nucleotides within a polynucleotide that is capable of forming a duplex with at least 8 nucleotides of a natural miRNA sequence.

As used herein, the terms “monotron,” “monotron sequence,” or “monotron element” are used interchangeably to refer a segment of a precursor RNA polynucleotide that is located at either the 5′ or 3′ end of the polynucleotide, i.e., either 5′ or 3′ from the intervening region. A monotron element refers to a sequence with 70% or higher similarity to a natural group I or group II intron including the splice site dinucleotide. In some embodiments, the monotron is capable of contributing to ribozymatic activity that allows it to enzymatically self-cleave. In some embodiments, the monotron is capable of forming a phosphodiester bond with a terminal sequence, i.e., a sequence containing a splice site dinucleotide and optionally a natural exon sequence or fragment thereof. In some embodiments, the terminal sequence is upstream of the monotron in a linear precursor. In some embodiments, the monotron sequence is upstream of the terminal sequence in a linear precursor. When the terminal sequence is upstream to the monotron in a linear precursor, the monotron can perform two transesterification reactions, e.g., sequentially, self-cleavage and formation of a phosphodiester bond with the terminal sequence. In embodiments in which the terminal sequence is upstream to the monotron in the linear precursor, (a) the monotron is capable of interacting with a nucleophile that is capable of cleaving at the splice site dinucleotide at or near the 5′ end of the monotron, and (b) the cleavage product of (a), i.e., the 5′ splice site nucleotide, e.g., having a 3′ hydroxyl group, engages in a transesterification reaction (cleaves) at the splice site nucleotide of the terminal sequence, yielding a circular RNA or oRNA. In these embodiments, the monotron interacts with the nucleophile (e.g., a guanosine, e.g., a free guanosine that is introduced to the precursor) by forming a binding pocket with the nucleophile, and the linear precursor is capable of adopting a conformation in which the nucleophile is in proximity to and is capable of cleaving at the splice site dinucleotide at or near the 5′ end of the monotron. When the monotron is upstream of the terminal sequence in a linear precursor, the monotron can also perform two transesterification reactions. In embodiments in which the monotron is upstream of the terminal sequence in the linear precursor, (a) the monotron is capable of interacting with a nucleophile that is capable of cleaving at the splice site nucleotide of the terminal element, and (b) the cleavage product of (a), i.e., the 5′ splice site nucleotide, e.g., having a 3′ hydroxyl group, engages in a transesterification reaction (cleaves) at the splice site dinucleotide at or near the 3′ end of the monotron, yielding a circular RNA or oRNA. In these embodiments, the monotron interacts with the nucleophile (e.g., a guanosine, e.g., a free guanosine that is introduced to the precursor) by forming a binding pocket with the nucleophile, and the linear precursor is capable of adopting a conformation in which the nucleophile is in proximity to and is capable of cleaving the splice site nucleotide of the terminal element.

In some embodiments, the monotron comprises a 5′ proximal end of a natural group I or group II intron including the splice site dinucleotide and optionally a natural exon sequence or fragment thereof. In some embodiments, the 5′ end of the monotron refers to nucleotides within the 5′ half of the monotron. In some embodiments, the 3′ end of the monotron refers to nucleotides within the 3′ half of the monotron. In some embodiments, at or near the 5′ end of the monotron refers to within the 5′ half of the monotron. In some embodiments, at or near the 5′ end of the monotron refers to within the first ten 5′ positions in the monotron. In some embodiments, at the 5′ end of the monotron refers to the first 5′ position(s) in the monotron. In some embodiments, at or near the 3′ end of the monotron refers to within the 3′ half of the monotron. In some embodiments, at or near the 3′ end of the monotron refers to within the last ten 3′ positions in the monotron. In some embodiments, at the 3′ end of the monotron refers to last 3′ position(s) in the monotron.

The term “nucleophile” refers to a nucleophilic nucleotide or nucleoside capable of initiating a nucleophilic attack at a splice site and/or transesterification reaction (cleavage) at a splice site.

The term “nucleotide” and “nucleoside” refer to a ribonucleotide, a deoxyribonucleotide, or an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Nucleosides are similar to nucleotides, e.g., comprising purines and pyrimidines, but without the additional phosphate group.

“Modified” nucleotide or nucleosides, or nucleoside or nucleotide “analogs” include nucleotides or nucleoside having modifications in the chemical structure of the base, sugar and/or phosphate, including, but not limited to, 5′-position pyrimidine modifications, 8′-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety as defined herein. Nucleotide or nucleoside modifications are also meant to include nucleotides or nucleoside with bases such as inosine, queuosine, xanthine; sugars such as 2′-methyl ribose; non-natural phosphodiester linkages such as methylphosphonate, phosphorothioate and peptide linkages. The “modified” nucleotide or nucleoside may be naturally occurring (e.g., pseudouridine) or synthetic. Nucleotide or nucleoside modifications include 5-methoxyuridine, 1-methylpseudouridine, and 6-methyladenosine. Exemplary nucleotide or nucleotide modifications are described herein. As exhibited by the exemplary nucleotide or nucleotide modification, such modifications differ from mutations selected from insertions, deletions, addition, or subtraction of nucleotides, for example, the mutations in a permuted Group I and Group II intron segment. As used herein, a nucleotide or nucleoside “comprising no nucleotide or nucleoside modifications” (i.e., comprising 0% modifications) can be interchangeable with “an unmodified nucleotide or nucleoside” in context. A modified polynucleotide sequence contains at least one nucleotide or nucleoside having a modification, e.g., between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides are modified. In some embodiments, “% modification” refers to the level of incorporation within a polynucleotide, i.e., the number of modified nucleotides or nucleosides in a polynucleotide sequence divided by the total number of nucleotides or nucleosides (modified or unmodified) in the polynucleotide sequence. In some embodiments, “% modification” refers to the relative quantity of modified nucleotide or nucleoside used to generate the polynucleotide (e.g., 5% modified adenosine refers to feeding 5 mM modified adenosine and 95 mM unmodified adenosine to generate a polynucleotide sequence).

All nucleotide sequences disclosed herein can represent an RNA sequence or a corresponding DNA sequence. It is understood that deoxythymidine (dT or T) in a DNA is transcribed into a uridine (U) in an RNA. As such, “T” and “U” may be used interchangeably herein in nucleotide sequences.

The terms “nucleic acid”, “polynucleotide”, and “nucleic acid molecule,” are used interchangeably herein to describe a polymer of any length, e.g., greater than about 2 bases, greater than about 10 bases, greater than about 100 bases, greater than about 500 bases, greater than 1000 bases, or up to about 10,000 or more bases, composed of nucleotides, e.g., deoxyribonucleotides or ribonucleotides, and may be produced enzymatically or synthetically (e.g., as described in U.S. Pat. No. 5,948,902 and the references cited therein), which can hybridize with naturally occurring nucleic acids in a sequence specific manner analogous to that of two naturally occurring nucleic acids, e.g., can participate in Watson-Crick base pairing interactions. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or azapyrimidines, pyrimidine bases with substituent groups at the 5 or 6 position (e.g., 5-methylcytosine), purine bases with a substituent at the 2, 6, or 8 positions, 2-amino-6-methylaminopurine, O6-methylguanine, 4-thio-pyrimidines, 4-amino-pyrimidines, 4-dimethylhydrazine-pyrimidines, and O4-alkyl-pyrimidines; U.S. Pat. No. 5,378,825 and PCT No. WO 93/13121). For general discussion see The Biochemistry of the Nucleic Acids 5-36, Adams et al., ed., 11th ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Naturally occurring nucleic acids are comprised of nucleotides, including guanine, cytosine, adenine, thymine, and uracil containing nucleotides (G, C, A, T, and U respectively).

As used herein, an “oligonucleotide” is a polynucleotide comprising fewer than 1000 nucleotides, such as a polynucleotide comprising fewer than 500 nucleotides or fewer than 100 nucleotides.

As used herein, “polyA” means a polynucleotide or a portion of a polynucleotide consisting of nucleotides comprising adenine. As used herein, “polyT” means a polynucleotide or a portion of a polynucleotide consisting of nucleotides comprising thymine. As used herein, “polyAC” means a polynucleotide or a portion of a polynucleotide consisting of nucleotides comprising adenine or cytosine.

As used herein, the term “ribosomal skipping element” refers to a nucleotide sequence encoding a short peptide sequence capable of causing generation of two peptide chains from translation of one RNA molecule. While not wishing to be bound by theory, it is hypothesized that ribosomal skipping elements function by (1) terminating translation of the first peptide chain and re-initiating translation of the second peptide chain; or (2) cleavage of a peptide bond in the peptide sequence encoded by the ribosomal skipping element by an intrinsic protease activity of the encoded peptide, or by another protease in the environment (e.g., cytosol).

The terms “sequence identity,” or “sequence similarity” as used herein, refers to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

As used herein, a “spacer” refers to a region of a polynucleotide sequence ranging from 1 nucleotide to hundreds or thousands of nucleotides separating two other elements along a polynucleotide sequence. The sequences can be defined or can be random. A spacer is typically non-coding. In some embodiments, spacers include duplex regions.

As used here, the term “splicing efficiency” refers to a measurement of the rate of splicing activity (e.g., none, low, or high) in a splicing or self-splicing reaction, for example, in portions of a precursor RNA polynucleotide capable of self-circularization. In some embodiments, the splicing activity of, e.g., a monotron element or intron segment, is affected by the structure and/or sequence of the linear RNA polynucleotide.

As used herein, “structured” with regard to RNA refers to an RNA sequence that is predicted by the RNAFold software or similar predictive tools to form a structure (e.g., a hairpin loop) with itself or other sequences in the same RNA molecule. As used herein, “unstructured” with regard to RNA refers to an RNA sequence that is not predicted by RNA structure predictive tools to form a structure (e.g., a hairpin loop) with itself or other sequences in the same RNA molecule. In some embodiments, unstructured RNA can be functionally characterized using nuclease protection assays.

As used herein, the term “therapeutic protein” refers to any protein that, when administered to a subject directly or indirectly in the form of a translated nucleic acid, has a therapeutic, diagnostic, and/or prophylactic effect and/or elicits a desired biological and/or pharmacological effect.

As used herein, “translation initiation element” or “TIE” refers to a portion of the intervening region comprising a sequence to allow translation efficiency of an encoded protein. In some embodiments, core functional elements comprising one or more coding elements will further comprise one or more TIEs. In some embodiments, where the intervening region comprises one or more noncoding elements, the TIE can be part of the noncoding element. In some embodiments, the TIE comprises an internal ribosome entry site (IRES).

As used herein, “transcription” refers to the formation or synthesis of an RNA molecule by an RNA polymerase using a DNA molecule as a template. The disclosure is not limited with respect to the RNA polymerase that is used for transcription. For example, in some embodiments, a T7-type RNA polymerase can be used.

As used herein, “translation” refers to the formation of a polypeptide molecule by a ribosome based upon an RNA template.

As used herein, the term “translation efficiency” refers to a rate or amount of protein or peptide production from a ribonucleotide transcript. In some embodiments, translation efficiency can be expressed as amount of protein or peptide produced per given amount of transcript that codes for the protein or peptide.

As used herein, the term “transfect” or “transfection” refers to the intracellular introduction of one or more encapsulated materials (e.g., nucleic acids and/or polynucleotides) into a cell, or preferably into a target cell. The term “transfection efficiency” refers to the relative amount of such encapsulated material (e.g., polynucleotides) up-taken by, introduced into and/or expressed by the target cell which is subject to transfection. In some embodiments, transfection efficiency may be estimated by the amount of a reporter polynucleotide product produced by the target cells following transfection. In some embodiments, a transfer vehicle has high transfection efficiency. In some embodiments, a transfer vehicle has at least about 10%, 20%, 30%, 40%, 50%, 60%, 70%, 80%, or 90% transfection efficiency.

As used herein, “transfer vehicle” includes any of the standard pharmaceutical carriers, diluents, excipients, and the like, which are generally intended for use in connection with the administration of biologically active agents, including nucleic acids. In certain embodiments of the present disclosure, the transfer vehicles (e.g., lipid nanoparticles) are prepared to encapsulate one or more materials or therapeutic agents (e.g., circRNA). The process of incorporating a desired therapeutic agent (e.g., circRNA) into a transfer vehicle is referred to herein as or “loading” or “encapsulating” (Lasic, et al., FEBS Lett., 312: 255-258, 1992). The transfer vehicle-loaded or -encapsulated materials (e.g., circRNA) may be completely or partially located in the interior space of the transfer vehicle, within a bilayer membrane of the transfer vehicle, or associated with the exterior surface of the transfer vehicle.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. The treatment or prevention provided by the method disclosed herein can include treatment or prevention of one or more conditions or symptoms of the disease. Also, for purposes herein, “prevention” can encompass delaying the onset of the disease, or a symptom or condition thereof, e.g., prophylaxis of disease.

As used herein, the terms “upstream” and “downstream” refer to relative positions of genetic code, e.g., nucleotides, sequence elements, in polynucleotide sequences. In some embodiments, in an RNA polynucleotide, upstream is toward the 5′ end of the polynucleotide and downstream is toward the 3′ end. In some embodiments, in a DNA polynucleotide, upstream is toward the 5′ end of the coding strand for the gene in question and downstream is toward the 3′ end.

As used herein, a “vaccine” refers to a composition for generating immunity for the prophylaxis and/or treatment of diseases. Accordingly, vaccines are medicaments which comprise antigens and are intended to be used in humans or animals for generating specific defense and protective substances upon administration to the human or animal.

### A. Lipid Definitions

As used herein, the phrase “biodegradable lipid” or “degradable lipid” refers to any of a number of lipid species that are broken down in a host environment on the order of minutes, hours, or days ideally making them less toxic and unlikely to accumulate in a host over time. Common modifications to lipids include ester bonds, and disulfide bonds among others to increase the biodegradability of a lipid.

As used herein, the phrase “biodegradable PEG lipid” or “degradable PEG lipid” refers to any of a number of lipid species where the PEG molecules are cleaved from the lipid in a host environment on the order of minutes, hours, or days ideally making them less immunogenic. Common modifications to PEG lipids include ester bonds, and disulfide bonds among others to increase the biodegradability of a lipid.

As used herein, the term “cationic lipid” or “ionizable lipid” refers to any of a number of lipid species that carry a net positive charge at a selected pH, such as physiological pH 4 and a neutral charge at other pHs such as physiological pH 7.

As used herein, the term “PEG” means any polyethylene glycol or other polyalkylene ether polymer.

As generally defined herein, a “PEG-OH lipid” (also referred to herein as “hydroxy-PEGylated lipid”) is a PEGylated lipid having one or more hydroxyl (—OH) groups on the lipid.

As used herein, a “phospholipid” is a lipid that includes a phosphate moiety and one or more carbon chains, such as unsaturated fatty acid chains.

As used herein, the term “structural lipid” refers to sterols and also to lipids containing sterol moieties. As defined herein, “sterols” are a subgroup of steroids consisting of steroid alcohols.

The terms “head-group” and “tail-group,” when used herein to describe the compounds (e.g., lipids) of the present disclosure, and in particular functional groups that are comprised in such compounds, are used for ease of reference to describe the orientation of such compounds or of one or more functional groups relative to other functional groups. For example, in certain embodiments, a hydrophilic head-group (e.g., guanidinium) is bound (e.g., by one or more of hydrogen-bonds, van der Waals' forces, ionic interactions and covalent bonds) to a cleavable functional group (e.g., a disulfide group), which in turn is bound to a hydrophobic tail-group (e.g., cholesterol). In certain embodiments, the compounds disclosed herein comprise, for example, at least one hydrophilic head-group and at least one hydrophobic tail-group, each bound to at least one cleavable group, thereby rendering such compounds amphiphilic.

As used herein, the term “amphiphilic” means the ability to dissolve in both polar (e.g., water) and non-polar (e.g., lipid) environments. For example, in certain embodiments, the compounds (e.g., lipids) disclosed herein comprise at least one lipophilic tail-group (e.g., cholesterol or a C₆₋₂₀ alkyl) and at least one hydrophilic head-group (e.g., imidazole), each bound to a cleavable group (e.g., disulfide).

As used herein, the term “hydrophilic” is used to indicate in qualitative terms that a functional group is water-preferring, and typically such groups are water-soluble. For example, disclosed herein are compounds (e.g., ionizable lipids) that comprise a cleavable group (e.g., a disulfide (S—S) group) bound to one or more hydrophilic groups (e.g., a hydrophilic head-group), wherein such hydrophilic groups comprise or are selected from the group consisting of imidazole, guanidinium, amino, imine, enamine, an optionally-substituted alkyl amino (e.g., an alkyl amino such as dimethylamino) and pyridyl.

As used herein, the term “hydrophobic” is used to indicate in qualitative terms that a functional group is water-avoiding, and typically such groups are not water soluble. In certain embodiments, at least one of the functional groups of moieties that comprise the compounds disclosed herein is hydrophobic in nature (e.g., a hydrophobic tail-group comprising a naturally occurring lipid such as cholesterol). For example, disclosed herein are compounds (e.g., ionizable lipids) that comprise a cleavable functional group (e.g., a disulfide (S—S) group) bound to one or more hydrophobic groups, wherein such hydrophobic groups may comprise, or may be selected from, one or more naturally occurring lipids such as cholesterol, an optionally substituted, variably saturated or unsaturated C₆-C₂₀ alkyl, and/or an optionally substituted, variably saturated or unsaturated C₆-C₂₀ acyl.

As used herein, the term “liposome” generally refers to a vesicle composed of lipids (e.g., amphiphilic lipids) arranged in one or more spherical bilayer or bilayers. Such liposomes may be unilamellar or multilamellar vesicles which have a membrane formed from a lipophilic material and an aqueous interior that contains the encapsulated circRNA to be delivered to one or more target cells, tissues and organs.

As used herein, the phrase “lipid nanoparticle” or “LNP” refers to a transfer vehicle comprising one or more cationic or ionizable lipids, stabilizing lipids, structural lipids, and helper lipids.

In certain embodiments, the compositions described herein comprise one or more liposomes or lipid nanoparticles. Examples of suitable lipids (e.g., ionizable lipids) that may be used to form the liposomes and lipid nanoparticles contemplated include one or more of the compounds disclosed herein (e.g., HGT4001, HGT4002, HGT4003, HGT4004 and/or HGT4005). Such liposomes and lipid nanoparticles may also comprise additional ionizable lipids such as C12-200, dLin-KC2-DMA, and/or HGT5001, helper lipids, structural lipids, PEG-modified lipids, MC3, DLinDMA, DLinkC2DMA, cKK-E12, ICE, HGT5000, DODAC, DDAB, DMRIE, DOSPA, DOGS, DODAP, DODMA, DMDMA, DODAC, DLenDMA, DMRIE, CLinDMA, CpLinDMA, DMOBA, DOcarbDAP, DLinDAP, DLincarbDAP, DLinCDAP, kLin-K-DMA, dLin-K-XTC2-DMA, HGT4003, and combinations thereof.

In some embodiments, a lipid, e.g., an ionizable lipid, disclosed herein comprises one or more cleavable groups. The terms “cleave” and “cleavable” are used in this regard to mean that one or more chemical bonds (e.g., one or more of covalent bonds, hydrogen-bonds, van der Waals' forces and/or ionic interactions) between atoms in or adjacent to the subject functional group are broken (e.g., hydrolyzed) or are capable of being broken upon exposure to selected conditions (e.g., upon exposure to enzymatic conditions). In certain embodiments, the cleavable group is a disulfide functional group, and in particular embodiments is a disulfide group that is capable of being cleaved upon exposure to selected biological conditions (e.g., intracellular conditions). In certain embodiments, the cleavable group is an ester functional group that is capable of being cleaved upon exposure to selected biological conditions. For example, the disulfide groups may be cleaved enzymatically or by a hydrolysis, oxidation or reduction reaction. Upon cleavage of such disulfide functional group, the one or more functional moieties or groups (e.g., one or more of a head-group and/or a tail-group) that are bound thereto may be liberated. Exemplary cleavable groups may include, but are not limited to, disulfide groups, ester groups, ether groups, and any derivatives thereof (e.g., alkyl and aryl esters). In certain embodiments, the cleavable group is not an ester group or an ether group. In some embodiments, a cleavable group is bound (e.g., bound by one or more of hydrogen-bonds, van der Waals' forces, ionic interactions and covalent bonds) to one or more functional moieties or groups (e.g., at least one head-group and at least one tail-group). In certain embodiments, at least one of the functional moieties or groups is hydrophilic (e.g., a hydrophilic head-group comprising one or more of imidazole, guanidinium, amino, imine, enamine, optionally-substituted alkyl amino and pyridyl).

### B. Chemical Definitions

The disclosure may include compounds and pharmaceutically acceptable salts thereof, pharmaceutical compositions containing such compounds and methods of using such compounds and compositions, and the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

Compounds described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; F may be in any isotopic form, including ¹⁸F and ¹⁹F; and the like.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, the term “alkyl” refers to both straight and branched chain C₁₋₄₀ hydrocarbons (e.g., C₆₋₂₀ hydrocarbons), and include both saturated and unsaturated hydrocarbons. In certain embodiments, the alkyl may comprise one or more cyclic alkyls and/or one or more heteroatoms such as oxygen, nitrogen, or sulfur and may optionally be substituted with substituents (e.g., one or more of alkyl, halo, alkoxyl, hydroxy, amino, aryl, ether, ester or amide). In certain embodiments, a contemplated alkyl includes (9Z,12Z)-octadeca-9,12-dien. The use of designations such as, for example, “C₆₋₂₀” is intended to refer to an alkyl (e.g., straight or branched chain and inclusive of alkenes and alkyls) having the recited range carbon atoms. In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

As used herein, “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

As used herein, “alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

As used herein, “alkylene,” “alkenylene,” and “alkynylene,” refer to a divalent radical of an alkyl, alkenyl, and alkynyl group respectively. When a range or number of carbons is provided for a particular “alkylene,” “alkenylene,” or “alkynylene” group, it is understood that the range or number refers to the range or number of carbons in the linear carbon divalent chain. “Alkylene,” “alkenylene,” and “alkynylene” groups may be substituted or unsubstituted with one or more substituents as described herein.

The term “alkoxy,” as used herein, refers to an alkyl group which is attached to another moiety via an oxygen atom (—O(alkyl)). Non-limiting examples include e.g., methoxy, ethoxy, propoxy, and butoxy.

As used herein, the term “aryl” refers to aromatic groups (e.g., monocyclic, bicyclic and tricyclic structures) containing six to ten carbons in the ring portion. The aryl groups may be optionally substituted through available carbon atoms and in certain embodiments may include one or more heteroatoms such as oxygen, nitrogen or sulfur. In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl).

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈ cycloalkyl,” derived from a cycloalkane. Exemplary cycloalkyl groups include, but are not limited to, cyclohexanes, cyclopentanes, cyclobutanes and cyclopropanes.

As used herein, “cyano” refers to —CN.

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

As used herein, “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” and “heterocyclic radical,” may be used interchangeably.

The terms “halo” and “halogen” as used herein refer to an atom selected from fluorine (fluoro, F), chlorine (chloro, Cl), bromine (bromo, Br), and iodine (iodo, I). In certain embodiments, the halo group is either fluoro or chloro.

As used herein, “oxo” refers to —C═O.

In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

In typical embodiments, the present disclosure is intended to encompass the compounds disclosed herein, and the pharmaceutically acceptable salts, pharmaceutically acceptable esters, tautomeric forms, polymorphs, and prodrugs of such compounds. In some embodiments, the present disclosure includes a pharmaceutically acceptable addition salt, a pharmaceutically acceptable ester, a solvate (e.g., hydrate) of an addition salt, a tautomeric form, a polymorph, an enantiomer, a mixture of enantiomers, a stereoisomer or mixture of stereoisomers (pure or as a racemic or non-racemic mixture) of a compound described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

In certain embodiments, the compounds (e.g., ionizable lipids) and the transfer vehicles (e.g., lipid nanoparticles) of which such compounds are a component exhibit an enhanced (e.g., increased) ability to transfect one or more target cells. Accordingly, also provided herein are methods of transfecting one or more target cells. Such methods generally comprise the step of contacting the one or more target cells with the compounds and/or pharmaceutical compositions disclosed herein such that the one or more target cells are transfected with the circular RNA encapsulated therein.

It is to be understood that the terminology used herein is for the purpose of describing particular embodiments only, and is not intended to be limiting. Unless specifically stated or obvious from context, as used herein, the term “or” is understood to be inclusive. Unless defined herein and below in the reminder of the specification, all technical and scientific terms used herein have the same meaning as commonly understood by one of ordinary skill in the art.

### 2. Precusor RNA, DNA Template & Circular RNA

The present disclosure is based in part on precursor RNAs comprising both 5′ intron and exon elements and 3′ exon and intron elements or comprising only 3′ exon and intron elements for producing circular RNAs with enhanced circularization efficiency.

Accordingly, provided herein is a precursor RNA polynucleotide capable of producing a circular RNA polynucleotide after splicing, wherein the precursor RNA polynucleotide comprises both 5′ intron and exon elements and 3′ exon and intron elements (e.g., combined accessory elements). Also provided is a precursor RNA polynucleotide capable of producing a circular RNA polynucleotide after splicing, wherein the precursor RNA polynucleotide comprises only 3′ exon and intron elements.

In some embodiments, a provided precursor RNA polynucleotide comprises (i) 3′ permuted intron segment comprising a 5′ nucleotide of a 3′ splice site dinucleotide; and (ii) a 3′ exon segment comprising a 3′ nucleotide of a 3′ splice site dinucleotide. Exemplary splice site dinucleotides are provided in the Table set forth herein. In some embodiments, a provided precursor RNA polynucleotide comprises (i) a 5′ exon segment comprising a 5′ nucleotide of a 5′ splice site dinucleotide; and (ii) a 5′ permuted intron segment comprising a 3′ nucleotide of a 5′ splice site dinucleotide. In some embodiments, a provided precursor RNA polynucleotide comprises a terminal element comprising (a) an excised terminal segment and a retained terminal segment or (b) a natural exon or a fragment thereof.

In some embodiments, a provided precursor RNA polynucleotide comprises (i) a 5′ intron element comprising a 3′ permuted intron segment comprising a 5′ nucleotide of a 3′ splice site dinucleotide; (ii) a 5′ exon element comprising a 3′ exon segment comprising a 3′ nucleotide of a 3′ splice site dinucleotide; (iii) a 3′ exon element comprising a 5′ exon segment comprising a 5′ nucleotide of a 5′ splice site; and (iv) a 3′ intron element comprising a 5′ permuted intron segment comprising a 3′ nucleotide of a 5′ splice site dinucleotide. In some embodiments, a provided precursor RNA polynucleotide comprises (i) a terminal element comprising (a) an excised terminal segment and/or a retained terminal segment or (b) a natural exon or a fragment thereof, (ii) a 5′ intron element comprising a 3′ permuted intron segment comprising a 5′ nucleotide of a 3′ splice site dinucleotide; and (iii) a 5′ exon element comprising a 3′ exon segment comprising a 3′ nucleotide of a 3′ splice site dinucleotide.

In some embodiments, a provided precursor RNA polynucleotide comprises a 5′ combined accessory element comprising (i) a 3′ permuted intron segment comprising a 5′ nucleotide of a 3′ splice site dinucleotide; and (ii) a 3′ exon segment comprising a 3′ nucleotide of a 3′ splice site dinucleotide. In some embodiments, element (ii) is located upstream to the intervening region. In some embodiments, the 5′ combined accessory element comprises a 3′ exon segment comprising a Group I or Group II exon 3′ nucleotide of a 3′ splice site dinucleotide.

In some embodiments, a provided precursor RNA polynucleotide comprises a 3′ combined accessory element comprising (i) a 5′ exon segment comprising a 5′ nucleotide of a 5′ splice site dinucleotide; and (ii) a 5′ permuted intron segment comprising a 3′ nucleotide of a 5′ splice site dinucleotide. In some embodiments, element (ii) is located downstream to the intervening region. In some embodiments, a 3′ combined accessory element comprises a 5′ exon segment comprising a Group I or Group II exon 5′ nucleotide of a 5′ splice site dinucleotide.

In some embodiments, a provided precursor RNA polynucleotide comprises a 5′ combined accessory element, an intervening region, and a 3′ combined accessory element. In some embodiments, (a) the 5′ combined accessory element comprises (i) a 3′ permuted intron segment comprising a 5′ nucleotide of a 3′ splice site dinucleotide; and (ii) a 3′ exon segment comprising a 3′ nucleotide of a 3′ splice site dinucleotide; and (b) the 3′ combined accessory element comprising (i) a 5′ exon segment comprising a 5′ nucleotide of a 5′ splice site dinucleotide; and (ii) a 5′ permuted intron segment comprising a 3′ nucleotide of a 5′ splice site dinucleotide. In some embodiments, the 5′ nucleotide of a 3′ splice site dinucleotide, 3′ nucleotide of a 3′ splice site dinucleotide, 5′ nucleotide of a 5′ splice site dinucleotide and 3′ nucleotide of a 5′ splice site dinucleotide are optionally a combination of nucleotides or a portion of a sequence selected from SEQ ID NOS: 2990-3668.

In some embodiments, the 5′ combined accessory element is located 5′ to the intervening region; and the intervening region is located is 5′ to the 3′ combined accessory element.

In some embodiments, a provided precursor RNA polynucleotide comprises a terminal element, an intervening region, and a monotron element. In some embodiments, the monotron element is located 5′ to the intervening region, which is located 5′ to the terminal element. In other embodiments, the monotron element is located 3′ to the intervening region, which is located 3′ to the terminal element. As set forth in further detail below, in some embodiments, the terminal element comprises a splice site nucleotide and the monotron element comprises a splice site dinucleotide and a splice site nucleotide.

In some embodiments, the precursor RNA polynucleotide is linear.

In some embodiments, permuted intron-exon splicing results in circularization of the precursor RNA polynucleotide. During splicing, a transesterification reaction can occur at the 5′ splice site and a second transesterification reaction can occur at the 3′ splice site. In some embodiments, splicing of the precursor RNA polynucleotide results in the removal of the 3′ intron element and the 5′ intron element. Accordingly, the circular RNA polynucleotide produced after splicing of the precursor RNA polynucleotide lacks the 3′ intron segment and the 5′ intron segment, but retains the 3′ exon segment and the 5′ exon segment.

In some embodiments, the precursor RNA polynucleotide is capable of circularizing when incubated in the presence of one or more guanosine nucleotides or nucleoside (e.g., GTP) and a divalent cation (e.g., Mg²⁺).

In some embodiments, the precursor RNA polynucleotide is between 300 and 10000, between 400 and 9000, between 500 and 8000, between 600 and 7000, between 700 and 6000, between 800 and 5000, between 900 and 5000, between 1000 and 5000, between 1100 and 5000, between 1200 and 5000, between 1300 and 5000, between 1400 and 5000, or between 1500 and 5000 nucleotides (nt) in length. In some embodiments, the precursor RNA polynucleotide is at least 300 nt, at least 400 nt, at least 500 nt, at least 600 nt, at least 700 nt, at least 800 nt, at least 900 nt, at least 1000 nt, at least 1100 nt, at least 1200 nt, at least 1300 nt, at least 1400 nt, at least 1500 nt, at least 2000 nt, at least 2500 nt, at least 3000 nt, at least 3500 nt, at least 4000 nt, at least 4500 nt, or at least 5000 nt in length. In some embodiments, the precursor RNA polynucleotide is no more than 3000 nt, no more than 3500 nt, no more than 4000 nt, no more than 4500 nt, no more than 5000 nt, no more than 6000 nt, no more than 7000 nt, no more than 8000 nt, no more than 9000 nt, or no more than 10000 nt in length. In some embodiments, the precursor RNA polynucleotide is about 300 nt, about 400 nt, about 500 nt, about 600 nt, about 700 nt, about 800 nt, about 900 nt, about 1000 nt, about 1100 nt, about 1200 nt, about 1300 nt, about 1400 nt, about 1500 nt, about 2000 nt, about 2500 nt, about 3000 nt, about 3500 nt, about 4000 nt, about 4500 nt, about 5000 nt, about 6000 nt, about 7000 nt, about 8000 nt, about 9000 nt, or about 10000 nt in length.

In various embodiments, provided herein are DNA templates that transcribe into precursor RNA polynucleotides of the disclosure. Accordingly, provided herein are DNA templates comprising sequences encoding the precursor RNAs of the disclosure. In some embodiments, the DNA template or polynucleotide of the present disclosure comprises a vector, a PCR product, a plasmid, a minicircle DNA, a cosmid, an artificial chromosome, a complementary DNA (cDNA), an extrachromosomal DNA (ecDNA), a doggybone DNA (dbDNA), a close-ended DNA (ceDNA), a viral polynucleotide, or a fragment thereof. In some embodiments, the polynucleotide of the present disclosure is selected from a DNA plasmid, a cosmid, a PCR product, dbDNA, close-ended DNA (ceDNA), and a viral polynucleotide. In some embodiments, the polynucleotide further comprises a promoter segment or sequence. In some embodiments, the DNA template is linearized. In other embodiments, the DNA template is non-linearized. In some embodiments, the DNA template is single-stranded. In some embodiments, the DNA template is double-stranded. In some embodiments, the DNA template comprises in whole or in part from a viral, bacterial or eukaryotic vector.

In various embodiments, provided herein is a circular RNA polynucleotide produced by circularization of a precursor RNA polynucleotide described herein.

In some embodiments, the circular RNA polynucleotide is produced inside a cell. In some embodiments, a provided precursor RNA is transcribed using a DNA template in the cytoplasm (e.g., by a bacteriophage RNA polymerase) or nucleus (e.g., by host RNA polymerase II) and then circularized.

In some embodiments, the circular RNA polynucleotide is between 300 and 10000, between 400 and 9000, between 500 and 8000, between 600 and 7000, between 700 and 6000, between 800 and 5000, between 900 and 5000, between 1000 and 5000, between 1100 and 5000, between 1200 and 5000, between 1300 and 5000, between 1400 and 5000, or between 1500 and 5000 nucleotides (nt) in length. In some embodiments, the circular RNA polynucleotide is at least 300 nt, at least 400 nt, at least 500 nt, at least 600 nt, at least 700 nt, at least 800 nt, at least 900 nt, at least 1000 nt, at least 1100 nt, at least 1200 nt, at least 1300 nt, at least 1400 nt, at least 1500 nt, at least 2000 nt, at least 2500 nt, at least 3000 nt, at least 3500 nt, at least 4000 nt, at least 4500 nt, or at least 5000 nt in length. In some embodiments, the circular RNA polynucleotide is no more than 3000 nt, no more than 3500 nt, no more than 4000 nt, no more than 4500 nt, no more than 5000 nt, no more than 6000 nt, no more than 7000 nt, no more than 8000 nt, no more than 9000 nt, or no more than 10000 nt in length. In some embodiments, the circular RNA polynucleotide is about 300 nt, about 400 nt, about 500 nt, about 600 nt, about 700 nt, about 800 nt, about 900 nt, about 1000 nt, about 1100 nt, about 1200 nt, about 1300 nt, about 1400 nt, about 1500 nt, about 2000 nt, about 2500 nt, about 3000 nt, about 3500 nt, about 4000 nt, about 4500 nt, about 5000 nt, about 6000 nt, about 7000 nt, about 8000 nt, about 9000 nt, or about 10000 nt in length.

Circular RNA polynucleotides lack the free ends necessary for exonuclease-mediated degradation, causing them to be resistant to several mechanisms of RNA degradation and granting extended half-lives when compared to an equivalent linear RNA. Circularization may allow for the stabilization of RNA polynucleotides that generally suffer from short half-lives and may improve the overall efficacy of exogenous mRNA in a variety of applications.

In some embodiments, the circular RNA polynucleotide has a functional half-life of at least 5 hours, 10 hours, 15 hours, 20 hours, 30 hours, 40 hours, 50 hours, 60 hours, 70 hours, or 80 hours. In some embodiments, the circular RNA polynucleotide has a functional half-life of 5-80, 10-70, 15-60, or 20-50 hours. In some embodiments, the circular RNA polynucleotide has a functional half-life greater (e.g., at least 1.5-fold greater or at least 2-fold greater) than that of an equivalent linear RNA polynucleotide comprising the same expression sequence. In some embodiments, the circular RNA polynucleotide, or a pharmaceutical composition thereof, has a functional half-life in a human cell greater than or equal to that of a pre-determined threshold value. In some embodiments, the functional half-life is determined by a functional protein assay. For example, in some embodiments, the functional half-life is determined by an in vitro luciferase assay, wherein the activity of Gaussia luciferase (GLuc) is measured in the media of human cells (e.g., HepG2) expressing the circular RNA polynucleotide every 1, 2, 6, 12, or 24 hours over 1, 2, 3, 4, 5, 6, 7, or 14 days. In some embodiments, the functional half-life is determined by an in vivo assay, wherein levels of a protein encoded by the expression sequence of the circular RNA polynucleotide are measured in patient serum or tissue samples every 1, 2, 6, 12, or 24 hours over 1, 2, 3, 4, 5, 6, 7, or 14 days. In some embodiments, the pre-determined threshold value is the functional half-life of a reference linear RNA polynucleotide comprising the same expression sequence as the circular RNA polynucleotide. In some embodiment, the functional half-life of a circular RNA polynucleotides provided herein in eukaryotic cells (e.g., mammalian cells, such as human cells) as assessed by protein synthesis is at least 20 hours (e.g., at least 80 hours).

In some embodiments, the circular RNA polynucleotide provided herein has higher functional stability than an mRNA comprising the same expression sequence. In some embodiments, the circular RNA provided herein has higher functional stability than an mRNA comprising the same expression sequence, 5moU modifications, optimized UTR, cap, and/or polyA tail.

In some embodiments, a provided circular RNA polynucleotide may have a higher magnitude of expression, e.g., a higher magnitude of expression 24 hours after administration of RNA to cells, than an equivalent linear mRNA. In some embodiments, the circular RNA polynucleotide has a higher magnitude of expression than an mRNA comprising the same expression sequence, 5moU modifications, optimized UTR, cap, and/or polyA tail.

In some embodiments, a provided circular RNA polynucleotide is transfected into a cell. In some embodiments, the DNA template, which transcribes into the precursor RNA polynucleotide from which the circular RNA polynucleotide is produced, is transfected into a cell and subsequently transcribed in the cell. Transcription of the circular RNA from the transfected DNA template may be induced via polymerases. In some embodiments, the polymerases are endogenous polymerases of the cell. In some embodiments, the polymerases are added to the cell. In some other embodiments, the polymerases are encoded by one or more nucleic acids transfected into the cell.

In some embodiments, the circular RNA polynucleotide is administered to an animal (e.g., a human) such that a polypeptide (e.g., an adjuvant, an adjuvant-like molecule, or an immunomodulatory molecule) encoded by the circular RNA polynucleotide is expressed inside the animal.

In some embodiments, a provided circular RNA is less immunogenic than an equivalent mRNA when exposed to an immune system of an organism or a certain type of immune cell. In some embodiments, the circular RNA is associated with modulated production of cytokines when exposed to an immune system of an organism or a certain type of immune cell. For example, in some embodiments, the circular RNA is associated with reduced production of IFN-β1, RIG-I, IL-2, IL-6, IFNγ, and/or TNFα when exposed to an immune system of an organism or a certain type of immune cell as compared to mRNA comprising the same expression sequence. In some embodiments, the circular RNA is associated with less IFN-β1, RIG-I, IL-2, IL-6, IFNγ, and/or TNFα transcript induction when exposed to an immune system of an organism or a certain type of immune cell as compared to mRNA comprising the same expression sequence. In some embodiments, the circular RNA is less immunogenic than mRNA comprising the same expression sequence. In some embodiments, the circular RNA is less immunogenic than mRNA comprising the same expression sequence, 5moU modifications, an optimized UTR, a cap, and/or a polyA tail.

Various circular RNA, circular RNA constructs, compositions comprising circular RNA, precursor RNA, and related methods are described, for example in U.S. patent application Ser. No. 17/853,576, WO2019236673, WO2020237227, WO2021113777, WO2021226597, WO2021189059, WO2021236855, WO2022261490, WO2023056033, WO2023081526, WO2023141586, and WO2023250375 which are each incorporated by reference in their entireties.

### A. Intron Elements, Exon Elements & Terminal Elements

Polynucleotides provided herein (e.g., DNA templates, precursor RNA polynucleotides, or circular RNA polynucleotides) may comprise one or more intron elements, exon elements, and/or terminal elements. In some embodiments, each intron element, exon element, and terminal element may independently comprise one or more spacers, intron segments, exon segments, duplex regions, affinity sequences, and/or untranslated elements. These sequence elements within the intron elements, exon elements, or terminal elements are arranged to optimize circularization and/or protein expression.

a. Intron and Exon Elements

In various embodiments, an intron element (e.g., 3′ intron element or 5′ intron element) comprises a permuted intron segment. In some embodiments, a 3′ permuted intron segment is a contiguous sequence at least 75% homologous (e.g., at least 80%, 85%, 90%, 91%, 92%, 93%, 94%, 95%, 96%, 97%, 98%, 99% or 100% homologous) to a 3′ proximal fragment of a natural intron (e.g., a group I or group II intron) including the 5′ nucleotide of the 3′ splice site dinucleotide. In some embodiments, a 5′ permuted intron fragment is a contiguous sequence at least 75% homologous (e.g., at least 80%, 85%, 90%, 91%, 92%, 93%, 94%, 95%, 96%, 97%, 98%, 99% or 100% homologous) to a 5′ proximal fragment of a natural intron (e.g., a group I or group II intron) including the 3′ nucleotide of the 5′ splice site dinucleotide. Exemplary splice site dinucleotides are described in the Table herein.

In some embodiments, an intron element comprises an intron derived from a trans-splicing ribozyme. In some embodiments, the intron element comprises a Group I trans-splicing ribozyme (e.g., a Tetrahymena trans-splicing ribozyme) segment. In some embodiments, the trans-splicing ribozyme segment along with an exon segment that may cleave a target site (e.g., a sequence of interest and/or a coding element) and subsequently ligate cleaved targe site to a 3′ exon to form a circular RNA product.

In various embodiments, a provided polynucleotide (e.g., a DNA template or a precursor RNA polynucleotide) comprises a 5′ exon element located upstream to the intervening region. In some embodiments, a provided polynucleotide comprises a 3′ intron element located downstream to the intervening region. In various embodiments, a provided polynucleotide comprises a 3′ exon element located upstream to the intervening region. In some embodiments, a provided polynucleotide comprises a 3′ intron element located upstream to the intervening region.

According to the present disclosure, the 3′ exon element and 5′ exon element each comprise an exon segment. In some embodiments, the 5′ exon element comprises a 3′ exon segment. In some embodiments, the 3′ exon element comprises a 5′ exon segment. In some embodiments, the 3′ and/or 5′ exon segment is a self-spliced or self-splicing exon segment. In some embodiments, the self-spliced and/or self-splicing exon segment comprises in part or in whole a naturally occurring exon sequence from a virus, bacterium or eukaryotic DNA vector. In other embodiments, the self-spliced and/or self-splicing exon segment comprises in part or in whole a non-naturally occurring sequence.

In some embodiments, a 3′ exon segment is a contiguous sequence at least 75% homologous (e.g., at least 80%, 85%, 90%, 91%, 92%, 93%, 94%, 95%, 96%, 97%, 98%, 99% or 100% homologous) to the 5′-proximal end of an exon adjacent a 3′ intron segment as described herein, including the 3′ nucleotide of the splice site dinucleotide. In some embodiments, a 5′ exon segment is a contiguous sequence at least 75% homologous (e.g., at least 80%, 85%, 90%, 91%, 92%, 93%, 94%, 95%, 96%, 97%, 98%, 99% or 100% homologous) to the 3′-proximal end of an exon adjacent a 5′ intron segment as described herein, including the 5′ nucleotide of the splice site dinucleotide.

In some embodiments, at least one of the exon segments is less than 15 nucleotides in length. In some embodiments, the 3′ exon segment and/or 5′ exon segment comprises a Group I exon segment or a Group II exon segment less than 15 nucleotides in length.

In some embodiments, the circular RNA comprises a self-spliced exon segment that is 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides. In some embodiments, the circular RNA comprises a self-spliced exon segment that is 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides from the exonic sequences of Table A (in which sequences are shown as 15-nucleotide exonic sequence, intronic sequence, 15-nucleotide exonic sequence), e.g., contiguous nucleotides from the 5′ or 3′ end of the exonic sequences of Table A. In some embodiments, the circular RNA comprises a self-spliced exon segment that is 5 nucleotides, 6 nucleotides, 7 nucleotides, 8 nucleotides, 9 nucleotides, or 10 nucleotides from the exonic sequences of Table B (in which sequences are shown as 10-nucleotide exonic sequence, intronic sequence, 10-nucleotide exonic sequence), e.g., contiguous nucleotides from the 5′ or 3′ end of the exonic sequences of Table B. See also SEQ ID NOs: 2990-3668, 25573, and 25574.

In some embodiments, the intron segment is a Group I intron and the exon segment comprises a Group I self-splicing exon segment. In some embodiments, the intron segment is a Group II intron and the exon segment comprises a Group II self-splicing exon segment.

In some embodiments, the exon element comprises a sequence directed to a native Group I intron-adjacent exon segment sequence or Group II intron-adjacent exon segment sequence, or fragment thereof. In some embodiments, the exon element comprises at least one mutation of a native Group I intron-adjacent exon segment sequence or Group II intron-adjacent exon segment sequence, or fragment thereof. In some embodiments, the exon element comprises at least one deletion of a native Group I intron-adjacent exon segment sequence or Group II intron-adjacent exon segment sequence, or fragment thereof. In some embodiments, the exon element comprises at least one insertion of a native Group I intron-adjacent exon segment sequence or Group II intron-adjacent exon segment sequence, or fragment thereof. In some embodiments, the native Group I intron segment or Group II intron segment sequences are selected from a sequence in Table A or Table B, below.

b. Terminal Elements

In various embodiments, a provided polynucleotide (e.g., a DNA template or a precursor RNA polynucleotide) comprises a terminal element. In some embodiments, the terminal element is located upstream to the intervening region. In some embodiments, the terminal element is non-intronic. In some embodiments, the terminal element lacks one or both nucleotides of a natural splice site dinucleotide associated with a natural Group I or Group II intron sequence. In some embodiments, a portion or the entire terminal element is excised after circularization of a precursor RNA polynucleotide comprising said terminal element.

In some embodiments, a polynucleotide comprises a terminal element, an intervening region, and a monotron. In some embodiments, the polynucleotide comprises, in the following order, a terminal element, an intervening region, and a monotron. In some embodiments, the polynucleotide comprises, in the following order, a monotron, an intervening region, and a terminal element. In some embodiments, the terminal element comprises a splice site nucleotide capable of engaging in a transesterification reaction with the monotron.

In some embodiments, the terminal element comprises an excised terminal segment and a retained terminal segment. In the same embodiments, the retained terminal segment is retained after circularization of a precursor RNA polynucleotide comprising such a terminal element. In the same embodiments, the exercised terminal segment is not retained after circularization of a precursor RNA polynucleotide comprising such a terminal element. In still the same embodiments, the nucleotide sequence of the terminal element is non-natural or synthetic.

In some embodiments, the terminal element comprises a natural exon or a fragment thereof. In some embodiments, the terminal element is retained after circularization of a precursor RNA polynucleotide comprising said terminal element.

In some embodiments, the terminal element is capable of binding to a 3′ intron element (e.g., the 3′ intron element comprised in the same polynucleotide). In some embodiments, the terminal element is capable of directing or functionalizing the splicing activity of a 3′ intron element (e.g., the 3′ intron element comprised in the same polynucleotide).

c. Exemplary Intron Elements, Exon Elements & Terminal Elements

For means of example and not intended to be limiting, in some embodiments, a 5′ intron element comprises, in the following 5′ to 3′ order: a 5′ leading sequence, an optional 5′ external duplex, a 5′ affinity tag, a 5′ external spacer, and a 3′ permuted intron segment. In the same embodiments, the 5′ exon element comprises, in the following 5′ to 3′ order: a 3′ exon segment, an optional 5′ internal duplex, and a 5′ internal spacer. In the same embodiments, the 3′ exon element comprises, in the following 5′ to 3′ order: a 3′ internal spacer, an optional 3′ internal duplex, and a 5′ exon segment. In still the same embodiments, the 3′ intron element comprises, in the following 5′ to 3′ order: a 5′ permuted intron segment, a 3′ external spacer, an optional 3′ external duplex, a 3′ affinity tag, and a 3′ lagging sequence.

As another exemplary embodiment, a terminal element comprises, in the following 5′ to 3′ order: a 5′ leading sequence, a 5′ external spacer, an excised terminal segment, a retained terminal segment, an optional 5′ internal duplex, and a 5′ internal spacer. In the same embodiments, the 3′ exon element comprises, in the following 5′ to 3′ order: a 3′ internal spacer, an optional 3′ internal duplex, and a 5′ exon segment. In still the same embodiments, the 3′ intron element comprises, in the following 5′ to 3′ order: a 5′ permuted intron segment, a 3′ external spacer, an optional 3′ external duplex, and a 3′ lagging sequence.

In some embodiments, the terminal element sequence has a percent sequence identity of about 70%, 75%, 80%, 85%, 90%, 91%, 92%, 93%, 94%, 95%, 96%, 97%, 98%, 99% or more to an exon fragment of a sequence selected from Tables A or B.

For means of example and not intended to be limiting, in some embodiments, a 3′ intron element comprises in the following 5′ to 3′ order: a leading untranslated sequence, a 5′ affinity tag, an optional 5′ external duplex region, a 5′ external spacer, and a 3′ intron fragment. In the same embodiments, the 3′ exon element comprises in the following 5′ to 3′ order: a 3′ exon fragment, an optional 5′ internal duplex region, an optional 5′ internal duplex region, and a 5′ internal spacer. In the same embodiments, the 5′ exon element comprises in the following 5′ to 3′ order: a 3′ internal spacer, an optional 3′ internal duplex region, and a 5′ exon fragment. In still the same embodiments, the 3′ intron element comprises in the following 5′ to 3′ order: a 5′ intron fragment, a 3′ external spacer, an optional 3′ external duplex region, a 3′ affinity tag, and a trailing untranslated sequence. In some embodiments, the affinity tag is a polyA affinity tag.

In some embodiments, the 5′ intron element is located 5′ to the 5′ exon element. In some embodiments, the 5′ intron element is adjacent to the 5′ exon element. In some embodiments, the 3′ intron element is located 3′ to the 3′ exon element. In some embodiments, the 3′ intron element is adjacent to the 3′ exon element.

In some embodiments, the 5′ exon element comprises a 5′ internal duplex sequence located 3′ to the 3′ exon segment. In some embodiments, the 3′ exon element comprises a 3′ internal duplex sequence located 5′ to the 5′ exon segment. In some embodiments, the 5′ intron element comprises a 5′ external duplex sequence located 5′ to the 3′ permuted intron segment. In some embodiments, the 3′ intron element comprises a 3′ external duplex sequence located 3′ to the 5′ permuted intron segment. In some embodiments, the 5′ intron element is adjacent to the 5′ exon element. In some embodiments, the 3′ intron element is located 3′ to the 3′ exon element. In some embodiments, the 3′ intron element is adjacent to the 3′ exon element.

In some embodiments, the 5′ intron comprises a 5′ affinity tag, a 5′ external spacer, and the 3′ permuted intron segment. In some embodiments, the 5′ exon comprises the 3′ exon segment, a 5′ internal duplex sequence, and a 5′ internal spacer. In some embodiments, the 5′ affinity tag is adjacent to the 5′ external spacer. In some embodiments, the 5′ affinity tag is located 5′ to the 5′ external spacer. In some embodiments, the 5′ internal duplex sequence is adjacent to the 5′ internal spacer. In some embodiments, the 5′ internal duplex sequence is located 5′ to the 5′ internal spacer. In some embodiments, the 3′ exon comprises a 3′ internal spacer, 3′ internal duplex sequence, and the 5′ exon segment. In some embodiments, the 3′ intron comprises the 5′ permuted intron segment, a 3′ external spacer, and a 3′ affinity tag. In some embodiments, the 3′ affinity tag is adjacent to the 3′ external spacer. In some embodiments, the 3′ affinity tag is located 3′ to the 3′ external spacer. In some embodiments, the 3′ internal duplex sequence is adjacent to the 3′ internal spacer. In some embodiments, the 3′ internal duplex sequence is located 3′ to the 3′ internal spacer. In some embodiments, the affinity tag is a polyA affinity tag.

In some embodiments, the 5′ exon comprises a 5′ internal duplex sequence located between the 3′ exon segment and the intervening region. In some embodiments, the 3′ exon comprises a 3′ internal duplex sequence positioned between the intervening region and the 5′ exon segment. In some embodiments, the polynucleotide comprises a 5′ internal duplex sequence and a 3′ internal duplex sequence.

In some embodiments, the 3′ and 5′ permuted intron segments each independently comprise a Group I intron segment, a Group II intron segment, a synthetic intron segment, or a variant thereof. In some embodiments, the 3′ permuted intron segment comprises a 3′ Group I intron segment or a variant thereof. In some embodiments, the 5′ permuted intron segment comprises a 5′ Group I intron segment or a variant thereof. In some embodiments, the 3′ permuted intron segment comprises a 3′ Group II intron segment or a variant thereof. In some embodiments, the 5′ permuted intron segment comprises a 5′ Group II intron segment or a variant thereof.

In some embodiments, the 3′ permuted intron segment or element, 5′ permuted intron segment or element, or both the 3′ and 5′ permuted intron segments or elements are at least 100, at least 90, at least 80, at least 70, at least 60, and/or at least 50 nucleotides in length. In some embodiments, the 3′ permuted intron element, 5′ permuted intron element, or both the 3′ and 5′ permuted intron elements are at least 50 nucleotides in length. In some embodiments, the 3′ permuted intron element, 5′ permuted intron element, or both the 3′ and 5′ permuted intron elements have a percent sequence identity of about 70%, 75%, 80%, 85%, 90%, 91%, 92%, 93%, 94%, 95%, 96%, 97%, 98%, 99%, or more to a naturally occurring intron.

In some embodiments, the 3′ permuted intron element, 5′ permuted intron element, or both the 3′ and 5′ permuted intron elements comprise a native Group I intron segment or Group II intron segment sequence. In some embodiments, the 3′ permuted intron element, 5′ permuted intron element, or both the 3′ and 5′ permuted intron elements comprise one or more nucleotide substitutions of a native Group I intron segment or Group II intron segment sequence. In some embodiments, the 3′ permuted intron element, 5′ permuted intron element, or both the 3′ and 5′ permuted intron elements comprise one or more nucleotide insertions of a native Group I intron segment or Group II intron segment sequence. In some embodiments, the 3′ permuted intron element, 5′ permuted intron element, or both the 3′ and 5′ permuted intron elements comprise one or more nucleotide deletions of a native Group I intron segment or Group II intron segment sequence. In some embodiments, the 3′ permuted intron element, 5′ permuted intron element, or both the 3′ and 5′ permuted intron elements comprise a nucleotide substitution of one or both the dinucleotide of a native Group I or Group II intron splice site dinucleotide. In some embodiments, the 3′ Group I or Group II intron segment or the 5′ Group I or Group II intron segment comprises one, two, three, four, five, six, seven, eight, nine, ten, or more mutations of a native Group I intron or Group II intron sequence. In some embodiments, the mutations are selected from insertion, deletion, mutation, addition, and subtraction. In some embodiments, the mutations are deletions of two or more nucleotides of the 3′ Group I or Group II intron segment or the 5′ Group I or Group II intron segment, or combinations thereof. In some embodiments, the mutations are two or more deletions of the 5′ Group I intron segment at the 3′ end or two or more deletions of the 3′ Group I intron segment at the 5′ end.

In some embodiments, the native Group I intron segment or Group II intron segment sequences are selected from a sequence in Table A or Table B, below. In some embodiments, the 3′ and/or 5′ permuted intron element comprise a polynucleotide sequence having a percent sequence identity of about 70%, 75%, 80%, 85%, 90%, 91%, 92%, 93%, 94%, 95%, 96%, 97%, 98%, 99%, or more to a naturally occurring intron selected from a sequence set forth in Table A or Table B, below, or a fragment or segment thereof. In some embodiments, the 3′ and/or 5′ permuted intron element comprise a polynucleotide sequence selected from a sequence set forth in SEQ ID NOS: 2990-3668. In some embodiments, the 3′ and/or 5′ permuted intron element comprise a polynucleotide sequence selected from a sequence set forth in SEQ ID NOS: 3188-3668.

In some embodiments, the 3′ permuted intron segment comprises a 3′ Group I or Group II intron segment derived from a gene selected from a genus and/or species selected from column 2 of Tables A or B; and/or the 5′ permuted intron segment comprises a 5′ Group I or Group II intron segment derived from a gene selected from a genus and/or species selected from column 2 of Tables A or B.

In some embodiments, the 3′ Group I or Group II intron segment or the 5′ Group I or Group II intron segment are derived from a gene selected from a species selected from: Cyanobacterium Anabaena sp., T4 phage, Hypocrea pallida, Bulbithecium hyalosporum, Myoarachis inversa, Geosmithia argillacea, Coxiella burnetii, Agrobacterium tumefaciens, Azoarcus, Nostoc, Cordyceps capitata, Prochlorothrix hollandica, Tilletiopsis orzyzicola, Tetrahymena thermophila, and Staphylococcus phage Twort.

TABLE A
Group I introns (flanked by 15 nt exons)

| SEQ | | |
|---|---|---|
| ID | | |
| NO: | Species | Sequence (15 nt exon-intron-15 nt exon) |
| 2990 | Anabaena_ | gaatggtagacgctacggacttaaataattgagccttagagaagaaattctttaagtggatgctctcaaactcagggaaacct |
| | sp | aaatctagctatagacaaggcaatcctgagccaagccgaagtagtaattagtaagttaacaacagataacttacagctaatcg |
| | | gaaggtgcagagactcgacgggagctaccctaacgtcaagacgagggtaaagagagagtccaattctcaaagccaataggcag |
| | | tagcgaaagctgcgggagaatgaaaatccgttgaccttaaacggt |
| 2991 | Hypocrea_ | gagggcaagtctggtaaacgttcgctggtgccagaaatagccagccctgtaaaatggctagtcgagtgctcgatctgtagcgg |
| | pallida | gtgagcaccggcgagacaacctggatcggggaagcctaagggacctgtcacagggtcctatggtgatcccgagcagagccgc |
| | | aaggcctgtgtagagcacgctaaggtgtcggtcccgtcctctgcggcggggcttaaggtacgtgccaatcccctgcgaaagct |
| | | gggccctagggagtagcacccgctgtgcgaagctctaggggacctgtgcccgccgtggcgcaggccgtccgacagtcgtca |
| | | gactgctcactataatggccagcagccgcggt |
| 2992 | Bulbithecium_ | gagggcaagtctggtgaactgttggtgccagagatagctgcggcccgcaacggcagctagtgggtgctcgatctgtgacgggt |
| | hyalosporum | gagcacctgcgagacaacctggatcgggggaccctaagggcgccacgtgcgcctatggaaatcccgagcagagccgcaag |
| | | gcctgtgtagagcgcgccaaggtgtcggtctgacttctgtcaggcttgaggtacgtgccgatccccagcgaaagctgggccca |
| | | gtggactagcacccgtcgtgcgaagccgctggggagtccgtgatggactttagcgacaaatcggccagcagccgcggt |
| 2993 | Mycoarachis_ | gagggcaagtctggtgaactgttggtgccagatagagccgcggccctgaacggcggctagtcgagtgcccgtcagtgacggg |
| | inversa | tgggcaccggcgagacaacctggatcgggggaccctaagggccgagaggcctatggaaatcccgagcagagcctgtagag |
| | | gcctgtgtagagcgcgccaaggtgtcggtccgtccccgcggggacgggcttaaggtacgtgccgaccccccgcgaaagcgg |
| | | ggcctaggggatgagcacccgtcgtgcgacgcccctggggagcatacgggctatgtataacggccagcagccgcggt |
| 2994 | Mito- | tagttgaatttgtttaacaagttccggagaatagtaatattctcatgaaaatcgggtgaattgtcaaggagacctcctacaat |
| | chondrion_ | ggagatgacttgcagcgaagcttatatcagtgcttaatagatataagaacgttcaacgactaatcggtgagtagcactaacaa |
| | Allomyces_ | taaaccggacactaggtggtgagtacaaccaacaataatccacccacgagcgcccgacaaccataattatggttgatgacata |
| | macrogynus | gtctgaacaggcaggtgactgtctgaagagaaatttaaacgatttctcgataacataattgttgtttgggg |
| 2995 | Geosmithia_ | ggcaccacaaggcgtaaaccattgctacagccgcagcaactctgcgccgaacagcagcccgaaagggtgatgtggtgccctc |
| | argillacea | taagcggtaaatgctagtctgcccccgggcaggcgacacgctcaaattgcgggaaacccctaaagctcccagctaccaagcc |
| | | gccggtcgaaagacggcggtggccaggttaattgcctcgggtacggtcacaacgctgggagatgcatgggcgatccgcagcc |
| | | aagcccctacgtccccccggggatacggggaaggttcagagactagatgggcgtgggttgaccgggaggggggcgtgtccc |
| | | cccccgggcggcttaagatatagtccggcaccgggagagatcccggtggtcaggtccacctgtaacacgggagcctgcggct |
| | | ta |
| 2996 | Stephano- | aggctgaaacttaaaggaattgacggaagggcaccaccaggagtaaacttttacagctagccgcagtaactctgctccaaaaa |
| | ascus_ | gcagcctgaaagggttagtggtgtcttcttttaattgctagtcgatatttttgtatcggcaacacgctcaaattgcgggaaat |
| | ciferrii | tcctaaaggctttattaccaagctaacattgaaaggtgttggtggccgagcgaaatgccctgggtatggtaaaaacataaagc |
| | | atgttacaatggatgatccgcagccaagctcctaagttcttgaaaaaaggatatggagaaggttcagagactaaatgggtgtg |
| | | aggtgtttgtacgcttaagatatagtccgtccatgactgaaaagtcaatggtggtaaccttaaaacgggagcctgcggcttaa |
| | | tttgactcaacacggggaaactcac |
| 2997 | Coxiella_ | tggggttagccctcgggcgaagctcttgatcgaagccccggtaaacggcggccgtaactataaattaaccgttgtagttacgt |
| | burnetii_ | aaataaacttggctatatgctggaaactccgagtatcctccagtactcatctcaactaaaattgaggtagtaaaaatctgcga |
| | RSA_493 | ggtgcggacaatcagcaggaaagactaatctttttgattagaatcctcagagactaatacgccgagcgtacttagaaaaaatc |
| | | cattctaagtgtgatgatagagtccgatctctatagcgatatagagggttttcaaaagaaacctgttaatcattatcccatga |
| | | ttaacataacgtcaataaacggtcctaaggtagcgaaattccttgtcgggtaaaaaatt |
| 2998 | Agro- | gagcacaggattcctaaggtaaattgggggttgcgcccggaaacgacgcaatcgatctgctcaaagtcggggaaagcttcgct |
| | bacterium_ | ggtcttccagtatgccaatcccgagccaagctccggagaaatccggtgaaggtgtagagactggatgggcagcacctaaaggc |
| | tumefaciens | cgcaaggtccacggtgaagggacagtccagaccacgaacgcccttatccgattgagatgggggcggcgaaagccgaagc |
| | | ggtatgaatcctggggtcgga |
| 2999 | Ankistro- | atgggtgaaagcactattgcccatcatagcagctgtccacaaagcagtctgctagtcgtcgttgcaccgctttgcaaggcgca |
| | desmus_ | acgcacggcgagaccgtcgaattgcggggacatccttacagctcaagctaccaacttgtcgcagaaatgctggcaagggccgg |
| | stipitatus | ggtaatgacccagggtatggtaaaaacgcttgagattggataatccgcagccaagcaccttacccggattcaacgtccggaaa |
| | | ggtgaaggttcagagactaggtggcggtcggtcacgagacgtggcttaagatagagtccgttcgcgctgaaaagcgttcgtcg |
| | | ggaagaaagccttagcaaggccggagccagacgaagggtagccattattggctgcctcgaggtacacgggtggtgc |
| 3000 | Monacro- | tttagaggaagtaaaagtcgtaacaaggtttgaagtatgacccttctgtggtgacacttacgagaagcctttgcggccccgca |
| | sporium_ | aggggtaccggccgcgactataaatgaaaggcaagccggcattaaatcgcaagtcagctcagctggctacactttcgaattgc |
| | leptosporum | gggaaacccctaaagccgacctctaccaactgttcagggaaacctggatggggcctgtgctaacatcacagggtacggtaaca |
| | | atgaggaggatatatgggcaatccgcagccaagtccctaaggctctcgagctatgggaaaggttcacagactaagtggaagtg |
| | | ggttggggcggttgtaatccgccccggcttaagatatagtcgggctgtccgcgagagcagacaggatgtcacaaatcaaccgt |
| | | tccgtaggtgaacctgcggaaggatcatta |
| 3001 | Nostoc_ | agataactgagccttgaaggagaaatccctcaagtggaagctctcaaactcagggaaacctaaatctggtgacagacatggca |
| | sp._F. | atcctgagccaagcccaaaaattttagatttgcgattagtcttaaattcaaaatctaaaatccaaaattacgggaaggtgcag |
| | | agacccgacgggagctaccctaacgtgaagtcgagggtaaagggagagtccaattctcaaaacctgatttggcgattgccagc |
| | | aagtagcagtgaaaactgcgggagaatg |
| 3002 | Nostoc_ | agataactgagccttgaaggagaaatccctcaagtggaagctctcaaactcagggaaacctaaatctggtgacagacatggca |
| | sp._C. | atcctgagccaagccgaaaaaagtcgtgagtgcagagtaaattaaaactcttaactcctaactattcggaaggtgcagagacc |
| | | cgacgggagctaccctaacgttaagtcgagggtaaagggagagtccaattctcaaaacctgatttggctattgctatcaggta |
| | | gcagtgaaaactgcgggaggatg |
| 3003 | Monora- | ggagaagtcgtaacaaggtttccgttgaattcactagcggataacagctgccctgaaaggggggctagtcattgatagctcat |
| | phidium_ | gatggtgagggctttcaattggcaacactgtcgaattgcgggaaattccttacagttctcactaccaagccttcgtggaaaca |
| | terrestre | cagtcggtggcctagggtaatcacctagggtatggtcaaaacgtgagggattggaaaatccgcagccaagctcctaaaggcga |
| | | catgacctatggagaaggttcagagactaggtggcagtgggctagttgctgcgtgcaattagtttaagatagagtccggccct |
| | | accgtgaggtagcctcaagacgaaaggactaaccagcccagagcttgaggagctgatgtgttacgtatcagtgggatcaaccg |
| | | aggtgaacctgcggaaggatcat |
| 3004 | Arxula_ | cagaaaagttaccacagggatgtgctaagtctattaagacaaatttacaacataacgtcccaggcatcgactcccattgatgc |
| | adeninivorans | ctagtccggctatagcttacttgtgcttgctatagttagctctctagaagagtgtaagggcaacacgtctggatgcggggaac |
| | | tctcgctaggtctttggtaccaaacattggcggtaacaccccaatgtagagtctgttggggcagacgggtaaaaatccaaaga |
| | | atagagacaatccgcagctgacctggccaaaagtatatttacaaagtagctaggcagttcaacgctcgctaagatgtgggttg |
| | | accattaatggtcggcttaaggtacgggctacgccacccgagagggtgcaactacctaccgggcgcacaacctgagaagtagt |
| | | tggtcatggtagtacggaactggcttgtggcagtc |
| 3005 | Penicillium_ | gaaagttaggggatcgaagacgatttaaggaagcctcccggcctggtttccgtggaaatcaggccggagatggtatttggtcg |
| | oblatum | tccaaagtaagcctgaaaggacttgctagtctcggccgtggccgaggcgacaccgtcaaattgcggggacctcctaaagcctg |
| | | gactaccaagccgacgccgaaaggcgccggtggccggggtaatgacctagggtatggtaacaacgtccgggatgtgacaatg |
| | | gacaatccgcagccaagcgctaccgcccctccggggaccacgcgtgcagttcacagactagatgtcggtgggggatccgtct |
| | | cctaagatatagtcgaggcccagcgcgaaagcctgggagtatccgcagataccgtcgtagtcttaaccataaactatgccg |
| 3006 | Staphy- | aactactgaaagcatAAATAATTGTGCCTTTATACAGTAATGTATATCGAAAAATCCTC |
| | lococcus | TAATTCAGGGAACACCTAAACAAACTAAGATGTAGGCAATCCTGAGCTAAGC |
| | phage | TCTTAGTAATAAGAGAAAGTGCAACGACTATTCCGATAGGAAGTAGGGTCAA |
| | Twort | GTGACTCGAAATGGGGATTACCCTTCTAGGGTAGTGATATAGTCTGAACATA |
| | | TATGGAAACATATAGAAGGATAGGAGTAACGAACCTATTCGTAACATAATTG |
| | | aacttttagttattt |
| 3007 | Cryptodia- | gagcatgggggcgaaagactaatcgaactgttgacgcacgagagtgtgggcacaccgtatgaaaattcgttttcgcgggctgc |
| | porthe_ | taggcagttaagagtccggccgtaaggcagggctagtaggcttcagagaggcctgcgacaccgtcaaattgcggggacacccc |
| | corni | attagaaagctgcgctaccgcctacggctcgaaagagctgtcaggcaccaggtgtaacggcctgggtatggtaataacgcgtg |
| | | tacgcaaggggcaatccgcagccaatccctaacccacctctataggtggtatggaggcagttcacagactagacggcggtggg |
| | | caccgttacagaaggtgcttaagatatagtcgagtcccgcctagcaataggtgggtagtttaacgctagtagctggttaccgc |
| | | cgaagtttccctcagg |
| 3008 | Pleurocapsa | acggacttaaaaaaattgagccttggcagagaaatctgtcatgcgaacgctctcaaattcagggaaacctaagtctggcaaca |
| | | gatatggcaatcctgagccaagccttaatcaaggaaaaaaacatttttaccttttaccttgaaaggaaggtgcagagactcaa |
| | | cgggagctaccctaacaggtcaagctgagggtaaagagagagtccaattctcaaagccagcagatggcagtagcgaaagctgc |
| | | gggagaatgaaaatccgt |
| 3009 | Spizello- | cgttgattacgtccctgcccttttccttgatggtacaaggggctaaatgtgctcccgaaaggaatgcgcaagtcttcagtggc |
| | myces_ | accgaaggcaacactgtcaaattgcggggaagccctaaagctttgtctaccaagcagggatggaaacatttctgtggccaggg |
| | punctatus | taatgacctcgggtatggtaaaaacggcaaagatagaacaatgggtaatccgcagccaagtcctaaacttgcacccagctggg |
| | | gcaagcatggatgcagttcacagactaaatggcagtgggctctgcgatagagattattgcggggcttaagatatagtcggacc |
| | | caccgcgagagcggttctctagatgaagctcagtgatttccaggtgcctgaagaactgatcctgagcccgagctagagtgtaa |
| | | agggagcatcgttgtacacaccgcccgtcgct |
| 3010 | Chlamydomonas | caggcttagttgtaggcctcctattaggcaactaatgggctaacattgggtgaattgcatgaaagctaaggcgtaacgctatg |
| | moewusii_ | ctaacctgcagcgaattacatcgactgatattaatataaaaatagatgtacacgttcaacgactagcgagtgatgtaaatcag |
| | Z17234 | tcaaataatctcgccacgagcgcccgacatcttgatatttacttagacccccgcttaagtaaatatggccaagatgatgacat |
| | | agtctgagcaacgtggaaacacgttggagccaaagataaaaaactttggcacatttctgaaacatgaaatgttcacgttaaaa |
| | | aaaataaaaacgtgaaaaacataactg |
| 3011 | Kirch- | ggaattgacggaagggcaccaccaggcgttaaaatagctctagcgccttagagtctcctcgagaggggaggctagtctgtagt |
| | neriella_ | ggctcacatcggtgagagcttctatcaggctacactgtcgaattgcggggaaatccttatagctcaagctaccaagccaggat |
| | aperta | ggaaacattctggtggcctggttaatgacccagggtatggtaacaacgcttgagattggacaatccgcagccaagcttcccac |
| | | caggtaatgctgaggaagacggttcagagactaagtggcagtggggggtgcaaacctgcttaagatagagtccgtccctaccg |
| | | aaaggtagctctgagaggaaaggcccaaccagcctaaagctcagagagcttgcttgagcaagtgggagtaaacgggagcctgc |
| | | ggcttaatttgactcaacacggg |
| 3012 | Pneumocystis | taaacggcgggagtaactatgactcaccttctgagggtcatgaaagcggcgtgaaaacgttagctagtgatctggaataaatt |
| | carinii | cagattgcgacactgtcaaattgcggggaagccctaaagattcaactactaagcagtttgtggaaacacagctgtggccgagt |
| | | taatagccctgggtatagtaacaatgttgaatatgaatcttttgcgagatgaaatgggtgatccgcagccaagtcctaagggc |
| | | atttttgtctatggatgcagttcaacgactagatggcagtgggtattgtaaggaattgcagttttcttgcagtgcttaaggta |
| | | tagtctatcctctttcgaaagaaagagtatattgtgctcttaaggtagccaa |
| 3013 | Panellus_ | ccaccaggtgtaaacttgttagccacagttaactctgcactaaaaagcagccctaacgggtgaggtggttcagccctagagtg |
| | stipticus | cgcaatgactcttaaatctaaatgctagtctgtgcatccttgtgttgcacgggcgagaccttcaaactgcgggaaactcctta |
| | | gagctttcactaccgctgctatcactgaaaagtgtagtctgcaccagggtaatgacctcgggtatggtaataacgtgaaagat |
| | | tggacaatccgcagccaagtccctaaaggtagttattgacctacgggaaaggttcagagactagatggaggttggtcaactaa |
| | | gttggcttaaggtatagtccgtctctgcatgaaagtgcaagaggtttgaaatgggagcctgcggctta |
| 3014 | Dunaliella_ | accaggcgttaaaaagcagactcagcgcctaagagtcagtgggaaaccattggctagtgcttgggtttcattactcaagtgca |
| | parva | acactgatcaaattgcgggaaagccctaaagctttgctaaccaagctaagtgtgaaagcactcagtggccgggttaaagacct |
| | | cgggtatggtaaaatcagcaaagatgcaacaatgggcaatccgcagccaagctcctgaagccttataggcaatggagaaggtt |
| | | cagagactaaatggcagtgggccaacttgttggcttaagatatagtccgtcccagctgaaaggctgtctgctagaggaagacc |
| | | tttctaggtctgagagctagtagagggtaggagaagtctctcctacctggaggaaacgggagcc |
| 3015 | Azoarcus_ | ctcatatttcgatgtgccttgcgccgggaaaccacgcaagggatggtgtcaaattcggcgaaacctaagcgcccgcccgggcg |
| | sp. | tatggcaacgccgagccaagcttcggcgcctgcgccgatgaaggtgtagagactagacggcacccacctaaggcaaacgcta |
| | | tggtgaaggcatagtccagggagtggcgaaagtcacacaaaccggaatccgt |
| 3016 | Ditopella_ | gagcatgggggcgaaagactaatcgaactgttagctactaggcagttaagagtctcccagtaatgggaggctagtaggctctt |
| | ditopa | tttcagagtttgcgacaccatcaaattgcggggacaccctgttataacgctatgataccgcgcctcgtggaaacacagagagt |
| | | agcactaggtgtaacggcctaggtatggtaaaaagtcattagctaggggcaatccgcagccaagccctacgtcttcggatatg |
| | | ggtgcagttcacagactaaacgttggtgggcttagaatctgtattctaggcttaagatatagtcgaatccgcctagtaatagg |
| | | tgggtatcaaatgaataggtgcaaaaacttgtttatttgactacagcagaaatgctagtagctggttaccgccgaagtttccc |
| | | tcaggatag |
| 3017 | Anabaena_ | cggacttaaataattgagccttagagaagaaattctttaagtggatgctctcaaactcagggaaacctaaatctagctataga |
| | sp. | caaggcaatcctgagccaagccgaagtagtaattagtaagttaacaacagataacttacagctagtcggaaggtgcagagact |
| | | cgacgggagctaccctaacgtcaagacgagggtaaagagagagtccaattctcaaagccaataggcagtagcgaaagctgcgg |
| | | gagaatgaaaatccg |
| 3018 | chlamydo- | gttatagtttatttgatggtactcttactcggataaccaagcaaaaggttagtccgtgtcagtttaaacgactggcaagtcga |
| | monad_ | ttctattggatcggcaacaccatcgaattgcgggaaagtccttagagccagtgctaccaagctggagtggaaacacaccggtg |
| | sp._NDem2_ | gccagggtaatgacctcgggtatggtaaaaacgcactggattggataatccgcagccaagctcctaagggtagcattgcctat |
| | 24P_3d | ctatggagaaggtccaacgactaagtgttggtgggctggtagcaaaccggcttaagatatagtctggccctgctgagaggcag |
| | | ccttgggattaaaggcttaaacagcccggagcccaaggggttggagcacatgctctgaccggaacaaccgaaccgtagtaatt |
| | | ctagagctaatacgtgcgtaaatcccg |
| 3019 | Azoarcus | ctcatatttcgatgtgccttgcgccgggaaaccacgcaagggatggtgtcaaattcggcgaaacctaagcgcccgcccgggcg |
| | sp | tatggcaacgccgagccaagcttcggcgcctgcgccgatgaaggtgtagagactagacggcacccacctaaggcaaacgcta |
| | | tggtgaaggcatagtccagggagtggcgaaagtcacacaaaccggaatccgt |
| 3020 | Penicillium_ | gaaagttaggggatcgaagacgatttagggccctcccggcgtccgccgacgccgggggatggtatttggtcgtcaaaagtaag |
| | sabulosum | ctgaaaaggtcttgctagtcccggccgcggccggggcgacaccgtcaaattgcggggactccctaaagcctgaactaccaag |
| | | ccgacgccgaaaggcgccggtggccggggtaatgacctagggtacggtaacaacgttcaggatgtgccaatgggtaatccgc |
| | | agccaagcgctaccgcccgtcccgggccacgcgtgcagttcacagactagatgtcggtgggggatccgtcccctaagatatag |
| | | tcgagccccggagcgaaagcccggggaggatccgcagataccgtcgtagtcttaaccataaactatgccg |
| 3021 | Ustilago_ | accaggagtaaactttcacagctagccgcagtgtctctgctccaaaaagccgcctgaaagggtcggtgcgtgttcctatttga |
| | maydis | aataatggctagtctgatgcgaatcaggcgacacatccaaattgcgggaaactccttagagctctagctaccgcggccccatc |
| | | gtgagattagggtgcagcaccaggttaatggcctcgggtatggtaaaaacgctagagattggacaatccgcagccaagcctct |
| | | tcgcaagaagtggaaggttcagagactaaatggttgtgggtggcagacggtttcgcgaatgtatgaatcaatcataaacacgt |
| | | gaaggaagtctgctgcttaagatatagtccgagccacagggaaaccttgggaattttggcaacaaaccgaaacagataaataa |
| | | catcgggagcctgc |
| 3022 | Pleurastrum_ | ccaggcgtaaaaccaagccgtttggctctgcgccagtcgggtgtgaggagggcctcaaacaagctcctcacacgagaaaccc |
| | paucicelulare | ggctagtcctggtgcggcttgcatcgcgccgccgcacctgggcaacatcatcgaattgctggaaacccctaaagctcttggta |
| | | ccaacctgcatgggaaaccgtgcgggggcccactccggggaggttataatccaagagatgcaacaatgggcaatcagcagcg |
| | | aagaccctgtcggttgggacttgcgtcccggcccatgggtaacgttcagagactaagtggtgatgggccccgatgccacccgg |
| | | cctcggggcttaagatatagtccggccccggcgaaagccgatgtcgagatgagggggtgcgaccccagagctcggcagtag |
| | | ggatcagagttcctgctctaatgtnggagcg |
| 3023 | Macropha- | agtacaaggaagcggaaggctataacaggtttaaaggcttgagtggcgcgaaagcgaccgctttgtggctacaaagtcgcgac |
| | ryngomonas_ | accgtcaaattgcgggaacaccctaaagcttcaactaccaagcgtatgtggaaacacttgcgtggccagggtaatgacctcgg |
| | halophila | gtatggtaaaaacgttgaagatgaaacaatgggcaatccgcagccaagccctaaagggatttttcctaagggtgcagttcaca |
| | | gactagatgtcggtgggtagagtcgtactctgcttaagatatagtcgaggccaccgtgaaaacggtgcacagtggaggaaatt |
| | | cggggtaactcctggatggagagccgctgtgggtagacaaaagtctactggtaacctgctgtaatgcccttagatgttctggg |
| | | ctgca |
| 3024 | Nostoc_ | agataactgagccttggaggagaaatccctcaagtggaagctctcaaactcagggaaacctaaatctgacaacagacatggca |
| | sp._A. | atcctgagccaagcccaaaaattttagatttgtgattttagatttacgattagtcttcaatccaaaattcaaaatctaaaatc |
| | | caaaattgcgggaaggtgcagagacccgacgggagctaccctaacgttaagtcgagggtaaagggagagtccaattcttaaaa |
| | | cctgagctggctattgccattaggtagcagtgaaaactgcggaagaatg |
| 3025 | Nostoc_ | agataactgagccttgaaggagaaatccctcaagtggaagctctcaaactcagggaaacctaaatctggttacagacatggca |
| | sp._Nephroma_ | atcctgagccaagcccaaaagttttagatttgcgattttagatttgcgattaatcttcaatccaaaattcaaaatcgttcgac |
| | resupinatum | tgagcgaagccgaagtctaaaatccaaaattgagggaaggtgcagagacccgacgggagctaccctaacgttaagtcgagggt |
| | | aaagggagagtccaattctcaaaatctgatctggctattgtcatcaggtagcagtgaaaactgcgggagaatg |
| 3026 | Nostoc_ | agacgctncggacttaaataattgagccttgaaggagaaatccctcaagtggaagctctcaaactcagggaaacctaaatctg |
| | sp._J | gtgacagacatggcaatcctgagccaagccgaagaaagtcctgagtcatgagtgctgagtgctgagtaaatttaaaactctta |
| | | actcctaactcctaactcataactgttcggaaggtgcagagacccgacgggagctaccctaacgttaagtcgagggtaaaggg |
| | | agagtccaattctcaaaatctgatctggctattgtcatcaggtagcaatgaaaattgcaggagaatgaaaatccgttgaccg |
| 3027 | Cordyceps_ | aattggagggcaagtctggtgaacgtcgctggcgccagagatagccgccctcggtaaacaaccggctagtcgagcgctcgttg |
| | capitata | cgtgacgggtgagcgccggcgagacaacctggatcggggaaggctaagggccccgcgggcctacgctgatcccgagcaga |
| | | gccacgcccggtggcctgtgtagagcgcgccaaggtgtcggtcccgaggtctcctcggggcttaaggtacgtgccaatcccct |
| | | gcgaaagcagggccctgcggaacagcacccgtcgtgcgaagccgcagggagcccccctccccgggggggcgtgtgctac |
| | | aggccagcagccgcggtaattccag |
| 3028 | Nodularia | acggacttagaaaactgagccttgatcgagaaatctttcaagtggaagctctcaaattcagggaaacctaaatctgtttacag |
| | | atatggcaatcctgagccaagccgaaacaagtcctgagtgttaaagctcataactcatcggaaggtgcagagactcgacggga |
| | | gctaccctaacgttaagccgagggtaaagggagagtccaattctcaaagccgaaggttattaaaacctggcagcagtgaaagc |
| | | tgcgggagaatgaaaatccgt |
| 3029 | Dactylella_ | gtcgtaacaaggttgaagtaagatccttctacagtgacacttacgagaagcctttgtggccccgcaaggggtacctgccgcga |
| | copepodii | ctataaacaatgcaagtgggtattaaatcgcaagtcagctttagctggccacactttcgaattgcggggaaaccctaaagctc |
| | | accataccaaccagcctgggaaatcaggttggggcccgtgctaactccacgggatatggtaataatgggatgagatgtaacaa |
| | | tgggcaatccgcagccaagtccctaaggtcttgtccagactacgggaaaggttcacagactaagtggaagtgggccggagttt |
| | | gtaccctccggcttaagatatagtcgggctgcctgtgagagcaagtgggaagtcacagttgcacttcaaaccgttccgtaggt |
| | | gaacctgcggaaggatcatta |
| 3030 | Prochloro- | agacgctgcggacttagataaactgggccttggtggagaaatccgcgaagtgtaagctctcaaattcagggaaacctaaggcc |
| | thrix_ | aagggctagggcaatcctgagccaagctgagggatcaaccacccctaggggcgttgaccttcggaaggtgcagagactcgacg |
| | hollandica | ggagctaccctaacaggccgtctgccaaggcaggcgatcgctgagggtaaagggagagtccaattctcaaaacctctggcac |
| | | cacgtcaggggcaacagtgaaagctgtgggagaatgaaaatccgttgaccg |
| 3031 | Protomyces_ | ttgacggaagggcaccaccaggagtaaacgtttatgtcgcagttaactctgctccgaaaagccactcgaaagagtgtggtgtc |
| | pachydermus | cttagaggctagtcagtccgctggcaacacaatcaaattgcgggaaactccttagagctcaagctaccgctgctgcccagaga |
| | | tgggctgtggcaccaggttaacgacctcgggtatggtaacaacgcctgagattggacaatccgcagccaagctccctcatggg |
| | | aagaaggttcagagactagatggttgtgggttgaacacgtttatctgaagtatttgaacggttcggcttaagatatagtccaa |
| | | tccaatgtggaaacgcactggtctgaaagatagaaaatgggagcctgcgcttaatttgac |
| 3032 | Monacros- | gtcgtaacaaggtttggaatataagatccttccgtgacgcttatgagaagcctttgtggccccgcaaggggtacccgccgcga |
| | porium_ | ctgtaaaaaaggatgcaggtattaaatcgcaagtcagcctttggctggccacactttcgaattgcggggatgccctaaagccc |
| | bembicodes | tcctctaccaaccaagctgggaaacctgcttggggcccgtgttaacggcacggggtacggtaacaacgaggttggataggtgg |
| | | gtaatccgcagccgagtccctaaggcaagccgctatgggaaaggttcacagactaagtggaagtgggctggatgctccagctt |
| | | aagatatagtcgggctgttggcgagagcgaacagatctgtcacttcatcatgattagtgcaaaaccgttccgtaggtgaacct |
| | | gcggaaggatcatta |
| 3033 | Tilletiopsis_ | gaggcaataacaggttaacctatcacaggcctgtaacagctgcgaggcagctagtccagcgtcgacgtctgcatttggggcag |
| | oryzicola | atggggtctgtttagaccttggcgcgggcgacacaacctggtacaggaaacgccccggcagcaatgccaggccgatcctgtg |
| | | gcgagctctgcgaggagccgtcgcaacgtgcggaaaggtgtgggtctttctaggaaaggcttaaggtacgtactaatccccgg |
| | | ggaaacccggcctctgcgagaagagcccaaactctatgcgcagggggaggcaacggtgttgccttggtatacatgctgtgatg |
| | | cccttag |
| 3034 | Protomyces_ | acggaagggcaccaccaggagtaacgtttatgtcgcagttaactctgctccgaaaagccactcgaaagagtgtggtgtcctta |
| | inouyei | gaggctagtcagtacgctggcaacacaatcaaattgcgggaaactccttagagctcaagctaccgctgctgcccagagatggg |
| | | ttgtggcaccaggttaacgacctcgggtatggtaacaacgcttgagattggacaatccgcagccaagctccctcatgggaaga |
| | | aggttcagagactagatggttgtgggttgaacacacgtttaattgaaacagttcagcttaagatatagtccaatccagtgtgg |
| | | aaacgcactggtctaaaagatagaaaatgggagcctgcggcttaatttga |
| 3035 | Artomyces_ | ttgacggaagggcaccaccaggagtaaacgcaacagcttagccgcagtggctctgctccgaacagcagcccgaaagggtga |
| | pyxidatus | ggtggtttaaaagccttgaggttcgatccgaggacctctaaagcgaaatgctagtctccctcgtggggggcgacaccttcaaa |
| | | ttgacggggaactcctaaagctcagagcaccaagcttcgttccgaaagggcggggtggccaggttaatagcctcgggtacggt |
| | | aaaagactctcgagatgttacaatggacgatccgcagccaaggccctacggcttcccttcgcggggtgagctacgggacaggt |
| | | tcagagactagatggaggtgggtcgtcgcgcgtcgacggcttaaggtatagtccgttcgcgagcgaaagctcgatgaggttta |
| | | ggtaaacccgcctttgacaaaacacggga |
| 3036 | Nostoc_ | agataactgagccttgaaggagaaatccctcaagtggaagctctcaaactcagggaaacctaaatctggtgacagacatggca |
| | sp._B | atcctgagccaagcccaaaaattttagatttgcgattagtcttaaatctaaaatccaaaattgcgggaaggtgcagagacccg |
| | | acgggagctaccctaacgttaagccgagggtaaagggagagtccaattctcaaaacctgagctggctattgccatcaggtagc |
| | | agtgaaaactgcgggagaatg |
| 3037 | Fusarium | ggcaccaccaggggtaaactacaacggtttctcgaaagccgcagttaactctgctcctaaaagccgctctaacgagttgatgg |
| | solani _f. | tgttccctggctcaggcctaaactaatggctagtctcctcggaggcgacatcctcaaactgcgggaaactcctaaagctcaca |
| | sp._phaseoli | ctaccaagcaggcctcgaaagagagctctgtggccggggtaacgacctagggtacggtaaaaacgtgtgagatgctacagtgg |
| | | acgatccgcagccaagcccctacgtgcgccagcatacggggaaggttcagagacttgacggggggggtgggctacaggcctgc |
| | | ctaagataaagtccggccccgcgtgaaagcgctgggggttacaactgcttctgcactattcgaaatactgcatcgaggcccca |
| | | gtaaaacgggagcctgcggcttaatttgact |
| 3038 | Nostoc_ | agataactgagccttgaaggagaaatccctcaagtggaagctctcaaactcagggaaacctaaatctggtgacagacatggca |
| | sp._I. | atcctgagccaagcccaaaaattttagatttgtgattttagatttatgattagtcttcaatccaaaattcaaaatctaaaatc |
| | | caaaattgcgggaaggtgcagagacccgacgggagctaccctaacgttaagtcgagggtaaagggagagtccaattctcaaaa |
| | | cctggccttgctaaagccatcaggtagcagtgaaaactgcgggagaatg |
| 3039 | Pnetmocystis_ | aactatgactcaccttttgagggtcatgaaagcggcgcgaaagtgttagctagtgatccgaaaaataaattcgggttgcgaca |
| | carinii | ctgtcaaattgcggggagtccctaaagattcaactactaagcagcttgtggaaacacagttgtggccgagttaatagccctgg |
| | | gtatagtaacaatgttgaatatgactcttaattgaggaaatgggtgatccgcagccaaatcctaaggacattttattgtctat |
| | | ggatgcagttcagcgactagacggcagtgggtattgtagagatatggggttatttatggccttatctacaatgcttaaggtat |
| | | agtctaatctctttcgaaagaaagagtagtgtgctcttaaggt |
| 3040 | Trebotxia_ | aggtttccgtaggtttnantatgcggataaaagcaggcttgaaaagagtttcctgctagtacctaactttttaagtaaggtgc |
| | gelatinosa | gacattgtcaaattgcctggaaatcctgtcaagcccctggtaccgctcatcttgaggaaactctggtgtgcacctcggtgaaa |
| | | gtcgagggtatggtaataatccagcgggataaggacaataggcagccaactgctaaagancccgaaggccctcangcaggcag |
| | | tccacagactaaatggcaatgggtcaaggagcattaactgctcattggcttaagatatagtcggtcctcgctgaaaagcggcc |
| | | aatgagaggaattctctttgagaaggagagctcattggagtttgggtaatactctgcaatgcaaattgtatgagggcaataag |
| | | cctccaaactgggtgaaacgaggtgaa |
| 3041 | Bionectria_ | aattgacggaagggcaccaccaggggttaacagaccgcagtcgctctgcccctaaaagcagccgggaaactggttaaggtgg |
| | atreofulva | gagtccttggccggaaggccgaaaactgctagtcatcgaacgatggcaacaccctcaaactgacgggaacgtcctaaagctga |
| | | tcaacaccaagcaagtgtccgaaaggtgcttgtggccgagctaatagccctgggtatggtaaaagcttgatcanatgctacaa |
| | | tggataactcgcagccaagcccctaagtagagcgatctatacggggaaggttcagagacttaacgggggtgggtgtggtgtan |
| | | accatgcctaagataaagtccgtccgcctgcgaaagcttgcgtagaaaaccgtaaaaacgggagcctgcggcttaatttgact |
| | | caacac |
| 3042 | Pleurastrum_ | gattctgtgggtgaacggtgtccgtgacactactgccccagcgggtgaaagcctacgctagtcgtccttgctcgacccaaatt |
| | paucic | gctatagcaagggaacggcgagaccatcgaattgcgggaaccccctaaagcttgcaccaccaaggcatgcgggaaaccgacat |
| | ellulare_ | gctggccagggtaatgacctcgggtacggtaaaagcgtgcaagatgcaacaatgggcaatccgcagccaagcgccctaacag |
| | SAG_463_1 | gtaatgctgagggtgaaggttcagagactaagtggtggtcggtgccggagcctgtgtgccccggtgcttaagatatagtccgg |
| | | cactacggaaacgtagccacgagaggaaaggccagcagcgcccggagctcgtggaggctgtcggtgctccgctcaccggga |
| | | ntgtgactaatcgatgtgattttgtgggtggtgcat |
| 3043 | Dunaliella_ | gacggaagggcaccaccaggcgttaacttagcagcaagctcagcgcctcaaagtcgaagggaaacctttggctagtatctggg |
| | salina | tgtagatttcacctaagtgcaacactgttcaaattgcgggaaagccctaaagctttgctaaccaagctgtcctagaaatggga |
| | | tggtggccaggtgaaagaccttgggtacggtaaaatcagcaaagatgcaacaatgggcaatccgcagccaagctcctacgggc |
| | | tgtcaaagcctatggagaaggttcagagactaaatggcagtgggcaagcatggcaatgcttgcttaagatatagtccgtccca |
| | | gctgagaagctgcctatgagaggaatgccgtaaggcaggagagctaataggaagtaagtgtctttaatcaacttacttggatt |
| | | ccacgggagcctgcggcttaatttgact |
| 3044 | Protomyces_ | cggaagggcaccaccaggagtaaacgtttatgtcgcagttaactctgctccgaaaagccgctcgaaagagtcatggtgtcctt |
| | macrosporus | gaaggctagtcagttcgctggcgacatttccaaattgcgggaaactccttagagctcaagctaccgcctctgtttagtgatag |
| | | acagctggcaccgggttaacgacctcgggtatggtaaaaacgtttgagattggacaatccgcagccagcttccttgagaggaa |
| | | gaaggttcagagactagatggatgtgggtgaagtctttggatatgaagattttgcttaagatatagtccaatccaaagcggag |
| | | acgcattggttacaacaagtagaaaatgggagcctgcgcttaatttga |
| 3045 | Planktothrix | acggacttaaagataaattgagccttgaggcgagaaatctctcaagtgtaagctgtcaaattcagggaaacctaaatctgtaa |
| | | attcagacaaggcaatcctgagccaagcctaggggtattagaaatgagggagtttccccaatctaagatcaatacctaggaag |
| | | gtgcagagactcgacgggagctaccctaacgttaagccgagggtaaagagagagtccaattctcaaagccaattggtagtagc |
| | | gaaagctacgggagaatgaaaatccgt |
| 3046 | Bionectria_ | aattgacggaagggcaccaccaggggttaacagaccgcagtcgctctgcccctaaaagcagccgggaaactggttaggtggg |
| | ochroleuca | aaagtccttggccggaaggccgaatactgctagtcatcgaacgatggcaacaccctcaaactgacgggaacgtcctaaagccg |
| | | atcaacaccaagcaggcgtccgaaaggtgcctgtggccgagctaatagccctggagatggtaaaagcttgatcggatgctaca |
| | | atggataactcgcagccaagcccctacgtgttccgacatacggggaaggttcagagacttaacggggggggtgtggtgtaga |
| | | ccatgcctaagataaagtccgtccgcctgcgaaagcttgcgaatacaaccgtaaaaacgggagcctgcggcttaatttgactc |
| | | aac |
| 3047 | Prochloro- | gtcaacccgtagaccatcaggggggcgtggcggaatggtagacgctgcggacttagataaactgggccttggtggagaaatc |
| | thrix_ | cgcgaagtgtaagctctcaaattcagggaaacctaaggccaagggctagggcaatcctgagccaagctgagggatcaaccac |
| | hollandica | ccctagggggttgaccttcggaaggtgcagagactcgacgggagctaccctaacaggccgtctgccaaggcaggcgatcgc |
| | | tgagggtaaagggagagtccaattctcaaaacctctggcaccacgtcaggggcaacagtgaaagctgtgggagaatgaaaatc |
| | | cgttgaccgtgaggttcgtgagggttcaagtccctccgcc |
| 3048 | Dactylella_ | gtcgtaacaaggtttgaagtaagtatccttccgagggtgacgctcacgagaagcctttgtggccccgcaaggggtacccgccg |
| | huisuniana | cgactgtaaagaatgaaagcaggtattaaatcgcaagtcagctctgctggccacactttcgaattgcggggacaccctaaagc |
| | | ctgcctctaccaaccgatcggggaaacctgaacggggcctgtgctaacgacacagggtacggtaacaacgaggaggatgcgaa |
| | | gaaatgggcgatccgcagccaacccctaaggcctgaaaaaggctacgggagaggttcacagactaagtggaagtgggctgga |
| | | ggtgttgaaaacctccggcttaagatatagtcgggccctgcgcgagagcgcgggggagtcacaactggactgaaccgttccgt |
| | | aggtgaacctgcggaaggatcatta |
| 3049 | Graphium_ | aaattgacggaagggcaccaccaggggttaacgtctaactgttaagccacagaagaaatctgctcctgaaagccgcccgaaag |
| | putredinis | ggtgaggtggtgtccggagcgtgcgagctcgaatttaaaggctagtcctccaatgcggagggcgacatcctcaaattgcgggg |
| | | aactcctaaagctttaactccaaagccggttcgcgaaagccgccggtggccaggttaacagcctcgggtactggaaaaacgtg |
| | | gaagatgcgacaatggacaatccgcagccaagcccctaaaggctccccgtcgagggacgcccacggggaaggttcagaga |
| | | cttaacggggatgggtattcgccgagaggcgcctgcttaagataaagtccggccctgcgcgagagcgcgggggtagcaggg |
| | | aactgcggcttaatttgactcaac |
| 3050 | Capronia_ | ggcgaaagactaatcgaaccattaagaagccactcggtggttgagtgtaggcccgaaagggtccgcaagtgccggatcctgg |
| | pilosella | cgcgacactgtcaaattgcgggaacaccctaaaactccagggaccgcctggcccccgaaagtgggccgaggcaccaggtta |
| | | acgacctcgggcacggtaatactcctggagatgtaacaatgggcaatctgcagccaagtcctacggggtcctccggggcctac |
| | | ggatgcagttcacagactagatggcagtgggctcggagagcttaagatatagtcggccccctggggagacccaggcgtgtcg |
| | | ctaaactcgaggtgaaggattccgcccccggcggccacctcgagcctgaacgacgcgggcctgtgttaaacaggtcggataa |
| | | gcgctggtagctggttcctgccgaagt |
| 3051 | Gaetmanno- | gggagtaactatgactcaagaattggcgaacgtgagagggtcatgaaagcggcgggaaaccggcgctagtgggcagccctg |
| | myces_ | cctgcgacattgtcaaattgcgggaacctcctaaagcctcgcccaccgcggcccgccgaaaggctgggtgcagcaccaggtta |
| | graminis_ | acgacctcggggacggtaatagcggcgaggatatgtggacaacctgcagccaagtcctaagttttggggtccgacgcgatgtc |
| | var._ | aagcgccggacctgggccaaaggcgaccgaaatagggatgcagttcaacgactagacggcaatgggtccccggggtgacg |
| | avenae | gccacggcgcttcgccgtcgtcccatacaaggggcttaagatatagtcttgtggtccgcttaacggcgtgtctgccacgtaaa |
| | | agctcttaaggtagcc |
| 3052 | Aspergillus | cgctagggatttataactgtgagtcctccaatattataaaatgttggtaatatattgggtaaatttcaaagacaacttttctc |
| | psudoglaucus | cacgtcaggatatagtgtatttgaagcgaaacttattttagcagtgaaaaagcaaataaggacgttcaacgactaaaaggtga |
| | | gtattgctaacaataatccttttttttaatgcccaacatctttattaactagttaataaagatgatgaaatagtctgaaccat |
| | | tttgagaaaagtggaaataaaagaaaatcttttatgataacataaattgaacaggctaa |
| 3053 | Arthrobotrys_ | gtcgtaacaaggtttgaagtttgttcccttccacaggtgacgctcacgagaagcctttgtgaccccgcaaggggtacccgccg |
| | cladodes | cgactgtaaagaatggaatgtgggtattaaatcgcaagtcagccgccgctggcaacactttcgaattgcgggaacaccctaaa |
| | | gcccacctctaccaaccagcctgagaaatccagctggggcccgtgttaacagcacgggaaatggtcataatgaggtgggatag |
| | | gtcagcctttccggttggcatcatggacaatccgcagccaagtccctaaggtccagctggggctacgggaaaggttcacagac |
| | | taggtggaagtgggttggttcagcaccaacttaagatatagtcgggctctgcgcgaaagcggaggggagtcactgttccgtag |
| | | gtgaacctgcggaaggatcatta |
| 3054 | Geosmithia_ | gaaagttaggggatcgaagacgatttaagaagggtccgcgccccccccggtgggggccggctcggtatttggtcgttaaaagt |
| | viridis | aagctcaaagtgtcttgctagtcgccgcgaccgcgggggcaacaccgtcaaactgcgggaacgtcctaaagcctgaactacca |
| | | agccgacgccgaaaggcgccggtggccggggtaatgacccagggtacggtaacaacgttcaggatgcgccaatggataacc |
| | | tgcaaccaagcgctaccgcctattggccatgcgtgcagttcacagactagatgtgggtggggggttgtaccccttaagatata |
| | | gtcgaagcccagtgtgaaagcctgggattagttgcagataccgtcgtagtcttaaccataaactatgccg |
| 3055 | Thiomargarita | aggattagatactacactaagtgtcccccagactggtgacagtctggtgtgcatccagctatatcggtgaaaccccattgggg |
| | namibiensis | taataccgagggaagctatattatatatatattaataaatagccccgtagagactatgtaggtaaggagatagaagatgataa |
| | | aatcaaaatcatcattaaagttatagaattatcagagaatgatatagtccaagccttatggtaacatgagggcacttgaccct |
| | | ggtag |
| 3056 | Mito- | ctgcgttagttgaatttgtttaacaagttccggagaatagtaatattctcatgaaaatcgggtgaattgtcaaggagacctcc |
| | chondrion_ | tacaatggagatgacttgcagcgaagcttatatcagtgcttaatagatataagaacgttcaacgactaatcggtgagtagcac |
| | Allomyces_ | taacaataaaccggacactaggtggtgagtacaaccaacaataatccacccacgagcgcccgacaaccataattatggttgat |
| | macrogynus | gacatagtctgaacaggcaggtgactgtctgaagagaaatttaaacgatttctcgataacataattgttgtttggggtggat |
| 3057 | Mito- | tacctcgactacggtacttttgtgccctgctttagcagtaaccgggttaattttgagaacatctaataagacaattcaaagcc |
| | chondrion_ | tatatataactgtataagttgaacgactattaataataattaactagaaaacctggctcctacttggagatgaaatagtctaa |
| | Trimorpho- | acattagtgtgaactaatgaaaccaataaaaggtgataatatgtattgacgatcagttaatgg |
| | myces_ | |
| | papilionaceus | |
| 3058 | Genicularia_ | aagtcgtaacaaggttttgccttgagcagacgcttgcggaagccttagcagcccacaagggttcccctcgcgactatatttaa |
| | spirotaenia | aggaaggggattgaatgctagtgcctgagctgccaacttgggtgcgacatcgccaaattgcggagaactccacatttgcctcg |
| | | attaccaagcggaccgccgaaaggaccccgtggccgggctaattgctttgggtatggtcaaaacatcgaggaaaaggatgatc |
| | | cgcagcgaaaccctcttgttgtacaaaagtgggtggcgttcacagactaaacggcgatgggttccagaaattcgaaacctggg |
| | | cttaagatgtagtcggtttgtcttcgaaagtggcagattgcagagtttcaccgttccgtaggtgaacc |
| 3059 | Oscillatoria_ | gtagctcgtcgggctcaatacgcaagttaactaaacgcttaatagttagcattgcgatgggctgcatactaaggaaactggta |
| | williamsii | tgcgatagcctctcaaattcggggaagccggtagcgcggtaatcccgagccaagctctgaacaagagaaggtgtagagactcg |
| | | atgggaggcaccctaacagttaaagctgagggtgaagggagagtccagaccacaaactggaaagataccgggcagcgaaagct |
| | | gtagatggtacgcataacccgaaggtc |
| 3060 | Anabaena_ | aaataattgagccttagagaagaaattctttaagtggatgctctcaaactcagggaaacctaaatctagctatagacaaggca |
| | sp | atcctgagccaagctgaagtagtaattagtaagttaacaacagataacttacagctaatcggaaggtgcagagactcgacggg |
| | | agctaccctaacgtcaagacgagggtaaagagagagtccaattctcaaagccaataggcagtagcgaaacgtgcggagaatg |
| 3061 | Oscillatoria_ | agaaaactgagccttattggagaaatccattaagtgaccgctctcaaattcagggaaacctaactctggtaacagacaaggca |
| | sp. | atcctgagccaagccgaaatttcggaaggtgcagagactcgacgggagctaccctaacgtaaagccgagggtaaagggagagt |
| | | ccaattctcaaaaccagaattctggcagcagcgaaagttgcgggagaatg |
| 3062 | Microcystis_ | acgcaacggacttagaccaaacttgagtgctcgcagggaaacctgcgatgcagaactgctcaaattcggggaaacctcttaac |
| | aeruginosa | tggcaatcccgagccaagccttggcgcgaaagtgcctcggaaggtgtagagactagacgggcagcacctaaaccccgccagc |
| | | ggggcacggtgaagggatagtccagaccacaaacattcgggctaccgagtggcggtgaaaaccgtagcaggtaagaaaatc |
| | | cgtcgggct |
| 3063 | Fischerella_ | cggtcaacggattttcattctcccgcagttttcactactgcctagtagactagctacttgtggctttgagaattggactctcc |
| | ambigua | ctttaccctcgacttaacgttagggtagctcccgtcgggtctctgcaccttccaaataagttgtttgttatttatcgcttgtc |
| | | ttttgttattcaacaattgacgaatgacaaatcacaaatgactaatttggcttggctcaggattgccttgtctgttgccagat |
| | | ttaggtttccctgaatttgagagcattcacttaaagagtttcttctttaaggctcaattacttaagtccgtagcgtct |
| 3064 | Synecho- | tcacgacctttacagtcagcggattttcattctctctgcaactttcgctgccatcaacaatcgcgatgttgagaattggacta |
| | coccus_ | tccctttaccctcggctgaatccgttagggtagctcccgtctagtctctgcaccttctaaagctgttagttgtttagctttag |
| | sp | cttggctcaggattgccatgactttaatgtttaaagttttaggtttccctgagtttgagagctgccactcgatccctttcgcg |
| | | atcgaggcccagttttctaagtccgctgcgtctaccgttccgccac |
| 3065 | Synecho- | cgacctttacagtcagcggattttcattctctctgcaactttcgctgccatcaacaatcgcgatgttgagaattggactatcc |
| | coccus_ | ctttaccctcggctgaatccgttagggtagctcccgtctagtctctgcaccttctaaagctgttagttgtttagctttagctt |
| | PC | ggctcaggattgccatgactttaatgtttaaagttttaggtttccctgagtttgagagctgccactcgatccctttcgcgatc |
| | C6301 | gaggcccagttttctaagtccgctgcgtctaccgttcc |
| 3066 | Chloroplast_ | agacgctacggacttaattggattgagccttggtatggaaacctactaagtgataattttcaaattcagagaaaccctggaat |
| | Gentiana_ | taataaaaagggcaatcctgagccaaatcctagttttcgaaaaagcaaaaagaaaggcttagaaagaaatgaaagaaataaag |
| | brachyphylla | gataggtgcagagactcaacggaagctgttctaacaaatggagttgattgcgttggtaaagaaacctttctaccaaaaattcc |
| | | aaaaggatgaggaaaaggggtatatacagactgaatcaaatgattcacccacaacctgcagatcttttcacgaacggattaat |
| | | cagacgagaataaagagagagtcccattctgcatgtcaatgccgacaacaatgaaatttatagtaagaggaaaa |
| 3067 | Mycos- | agggcaagtctggtggacgtccagtcccaatgctggcgccagaagcagtgaggccccaactcactagtcgatgcacccacca |
| | phaerella_ | acagagagggcgggtgccggcaagacgacctggtacgagggaccccttaacggccgcctataggtggggccgaaggcaat |
| | juvenis | ctcgtggcgacctccctcacacggaggcgtcgtaacgcgcggaaaggcgtcggtcgcgataagcggcttaaggaacgtgcta |
| | | gacccgagggaaacctcgctccttctgatcagccccctctcggcgaagcagtggaggcgcccggcgaacgggtgtggacga |
| | | gtcaatgaaaatcggccagcagccgcggta |
| 3068 | Barrmaelia_ | agggcaagtctggtgaactgaactcagcttccagctggcgccagagatagtggttaaaatcactagtcgagcatcctctccca |
| | oxyacanthae | cggtgaggacgccggcaaggcaacctggattcggggaaggctaaggggcttacgggccctatgccaatcccgagcagaaatta |
| | | cttctgtgtagagcgcggaaaggtgccggtcgtgagacctaccctggaacagcctaccctgtagcaagttaccctgtaacgag |
| | | ttaccctgtaggccttctcggttggggctcgcggctcgagggacgtgccaaccacctacgaaagtaggttctagtatcccgag |
| | | cgtagagctaggggtatggagccctgtattttagggtcgtcagtcactgatagtggccagcagccgcggta |
| 3069 | leotiomycete_ | ggagcctgcggcttaatttgactcaacacggggaaactcaccaggttaactacagttacgtctgggcctggaatagttatttg |
| | sp. | acaactagtacacgcctttacttgtgtgggagaagtctcccaattttggacacaaggcctttcaggagggtttggcggtgcga |
| | | cactacctggtacaggggacgcctcgctggagtgatccggcaggccgactctgtggcgagctcgagtaacgtcgagccgtcgc |
| | | aacggcgggaaaggagtgggttcggtggatct |
| 3070 | Tilletiopsis_ | gaggcaataacaggttaacctatcacaggcctgtaacagctgcaaggcagctagtccagcgtcgacgtctgcgtttgaggcag |
| | flava | atggggtcttttggaccttggcgcgggcgacacaacctggtacaggaaacgccccggcagcaatgccaggccgatctgttggc |
| | | gagctctgcgaggagccgtcgcaacgtgcggaaaggtgtgggtctttctctatgaaaggcttaaggtacgtactaatccccgg |
| | | ggaaacccgggcctctgcgagaagagcccaaactctatgcgcagggggaagcttgcggaatgcaaagctttggcgatgatcct |
| | | ttgtgacatcgataaatgctgtgatgcccttag |
| 3071 | Papulosa_ | cgatggaagttcgaggcaataacaggtcaacgcatcgcaggcctgtaatagtttaaataacgctagtccgctggatttacagc |
| | amerospora. | gggcgacactaccttgtacaggggacgcagtgctcgtcttgggagtctgccaatcctgtggcgagccctttgggccgtcgcaa |
| | | cgcgcgcagggagtgggctggggcttattggctccggcttaagatacgtgctaagctactgggtgaaaaccaggcttcgcgct |
| | | taaaaacgcggaggatttcgtgacgaacttagaaatagactgctgtgatgcccttagatgttctgggccgcacgcgcgt |
| 3072 | Chloroplast_ | cagcagccgcggtctggcgtaactaatagaagttgcaatgccgccatagcgagtgatcgttatgtaaaaaaaccctgctcata |
| | Chlamy- | acggtgaagccttagtaatttatctttttagggtattttacggtaataccgtgggaagtttatcaacaaaaggctgcccgcgg |
| | domonas_ | caaagccgcgggcacaaaaaaagcttttttaaaacgtgttgatataaccccgtaacgacttttcttgaaagagaaagaggtgc |
| | moewusii | tttattagcgcctaacacgcaggcacttacgagtcctaaatgcagcccgcggaaaccgcgggcggctatggtaataatctagc |
| | | ccgcggctttgccgcgggcgatgcctaaagtctaaagtaagttgaaggtatagtctgagcctctggaaacaggggaataatcg |
| 3073 | Chlamy- | gcccattaaagtggtacgtgagctgggttcaaaacgtaaataacactgcgtgtagttgtaaaaatacaactaaaagatttggc |
| | domonas_ | ttatatcggcgaaaccttactatctttttaactgtacgatagatggcaatgccgagggaagttcttattctaaattaatagat |
| | callosa | taagacacctgtagagactttataagtttataaaacttataagatacttcctacaaaggaaagtataatacgccaacccctac |
| | | tttttacctgagcttataaacaagcgaaaggctaaagcggggaggagatatagtccatgctcttatgaaaataggagggaagg |
| | | tttaacaatatttgttaaactgcatgcgtgagacagttcggtccatatccg |
| 3074 | Amoebidium_ | ggaaagtagacgcgttccggtgaagagtgttctacccttacgacaaaccgctcgaatgggccttgtgagagacaactctcaca |
| | parasiticum | ttgcagttggctatatgctagaaactaccgttgtattcataaatacaaaatgttatctactcttattgtatatgggagtgaca |
| | | atgatggacacgaggtacaattagcaggaaaccaaaggttacaagggtaacttagtaggatcttcagagactacacgccaacc |
| | | atcttgaattagcaagatgaagacatagtccatagtggattagtaaagatactagtattaatattaatctactagcgtcaagc |
| | | tgagaaagggttagttcgatgaagacaatcagtgttaagg |
| 3075 | Chara_ | agcgaaattcctcgacaaaggggtattttctgaacagcatcaaagacaaaatctggctatatgctggaacctccgagtgacgc |
| | vulgaris | aagcaaaatgcgtccggacaatcagcagggaaggaagtcatacatgtgttgatacatgcgctgcgcccctcaacgactacacg |
| | | ccagacatccctgcttgggatgatgatatagtctgaacttcgtggcgacacgaaggaggaaggcctcggttgaaatgatgttc |
| | | gaagtttatttcaattaacacaattgtgtcgcataagtagcgacctgcacgaatggtgtaacg |
| 3076 | Mito- | cgacgcgcgtgttcggcaccttgaggcgctacgcaagcgtagcgccgattcatcggggtatttttatagtgatataaaaaaaa |
| | chondrion_ | gaaaaaggctaaaagctggaaaatctataatatgataatcagctggaacaaaagtatgtaataagtaatagttattatatata |
| | Chlamy- | aaagattcttcaacgattaaaagcctagctcttttaataaagagtatgatgtagtctgatc |
| | domonas_ | |
| | moewusii | |
| 3077 | Pyrictlaria_ | atttagccgaggctaaaaaaatattctgatttggtctggttatatgtcgcaaattcctggaaaatctttataaggtaatcagc |
| | higginsii | aggaaaacttaattaatttagtgattaaactcttcatcgactaaacgtgacaatttaatttttttattaaataagagatagtc |
| | | aaaatttcgatgtgaatcgaataatatttattataagagcaccaggttcccagagcgctaggcgattattataatttatatta |
| | | gccccccttggaatcag |
| 3078 | Stigeo- | agtgaacggcggccgtaactatgacggtcaactttagggccgtctttaaagataactttttgctggaacctccataaaaagag |
| | clonium_ | caatcagcaagagattatattatttgttctaataatttcaacatctgtataaatttccgtctacattaaatggcttactttac |
| | helveticum | gaatgtcttcctttatgggatgccatgagaatacaaaaaggccaaaaaaatcaaagttttgaaactcttgaagacgcgcaaaa |
| | | ttatgtacgagattttgttgcgaacaaataaaaatagtttcttcagagactactggttatccgattttaaatattttagtatt |
| | | taagattgaagatatagtccgatattatatgaatgtgtaataaataccttgcctaaggtagcgaaattctttgtcgagtaag |
| 3079 | Neochloris_ | ggcagctgtaactatgacagttctaaggttaagaaatgctagctttagtaaaatcaaactatttgctgggaacccgtaataaa |
| | aquatica | ggaaatcagcaggtaactctataataatttatttatagagcacctcagagactatacgtttgacgttttgatgaaaacatcaa |
| | | ctttaagatagagtccagcgttttttgaaagaataacgattatctgagcgaaattcattgtcgagtaagtttcg |
| 3080 | Pedinomonas_ | caggctgatcttccccaagagttcacatcgacgggaaggtttggcacctcaactggggtctatcaatgtgaattgataatgca |
| | tuberculata | caagttagctatatgctggaaaatccggaggatccaaaaacccaccctaattttgttaggtcggaaaagttttttggttcgga |
| | | caatcagcagggaagaccatgtgcccctcaacgactacacgctgacctacttaaaaaaaaagtagatgatatagtctgaactt |
| | | caaggcgacttgaaggtattcatttttctgtgaacgaactgcaactagcagcacgcgaaaaggaacacgttttcgattgcatt |
| | | cgaaaattaacgtctttgcgatgtcggctcatcgcaacctggggcggtagtacgtcccaagggttgggctgttc |
| 3081 | Chlamy- | caggctgatcttccccaagagttcacatcgacgggaaggtttggcacctcgacatagggggtcgctttcacgcaagtgaaggc |
| | domonas_ | tgtaaattagctgtatgctggaacttacctattaggtaaaatcagcaggaagtgaagcacacgtgtttagctcctcaacgact |
| | starrii | acatgctaacactcttgtccttgcgtcctgtgccgcgccgcgccttcttgtccttcgccgaaggcgcggacgcgaaggcgaag |
| | | gcaaggacgcgaaggggaaaggacgcaagagcagagtgatgatatagtctgaactacgctttaacggcatagaccaacttagt |
| | | tggtttctttctagataacctaggagggataacatatttgcgatgtcggctcatcacatcctcggtctgtagtaggtccgaag |
| | | ggttgggctgttc |
| 3082 | Chara_sp._ | gatcttccccaagagttcacatcgacgggaaggtttggcacctcaaatcggggcaatagtccagtaatgggaaaataaagtag |
| | CL_200 | agaatttggctatatgctgggacatccgtagtttctaatctaaaaaaataagtgacaatagcgtaggaaatgaattgaatgaa |
| | 2_1 | ttcggattatcagcagggaagtttcttcaagatacccctcaacgactacacgccatgtatcctttgcttttcaaggataatga |
| | | tagagtctgagcttttaggtaactaaaaggccactattgagtgttagtggcgttaaaccatacatgtcgaaattccccggaaa |
| | | attgataatgtttaattaacacattgcgatgtcggctcttcgccacctggagcggtagtacgttccaagggttgggctgttc |
| 3083 | Chara_ | gatcttccccaagagttcacatcgacgggaaggtttggcacctcaaatcggggcaatcgtccagtaatgggaaaataaagtag |
| | corallina | agaatttggctatatgctgggacatccgtagtttctaatcaaaaaaatcagtaacaatagcgtaggaaatgaattgaatgaat |
| | | tcggattatcagcagggaagtttcttcaagatacccctcaacgactacacgccatgtatcctttgcttttcaaggataatgat |
| | | agagtctgagcttttaggtaactaaaaggccactattgagtgtgagtggcgttaaaccatacatgtataaattacccggacta |
| | | aaaatccataaaaaaatcaaattgagaatgtttaattaacacattgcgatgtcggctcttcgccacctggagcggtagtacgt |
| | | tccaagggttgggctgttc |
| 3084 | Selenastrum_ | cctcttacacggataaccgtataatacatgcggaagaagtactcgcgttcctagtagccttaaaaggctgcgacattgcaatt |
| | capri- | gcctgaaccctgttaatctcaccagtaccgctgctgtcccgaaagggaccacgcagcaccttggcaacgcccgagggtgtggt |
| | cornutum | aaaaacctgaagagtaggggcgatgggcagccaagtcctgttggtagccaatacctacggatgcagttcacagactaaatggt |
| | | aatgggttcttgagtttgactacacaagagcttaagatatagtcggtgggtgccaaaaggcagaccgcaggaggacaacaacc |
| | | acacgttgtctgagagcctgtggtggacagtaatagttgggtgcaaccaaccggtctgaccgtctgctcccggaacaaacgag |
| | | taattctagagct |
| 3085 | Chlorella_ | tactcggataaccgtaaggattnttgcgggagggaancgaaagcctgccctagtggctcgaaaagcgagctgcgacactgtcg |
| | trebotxioides | aattgcccggacgtcctgctacgctggagggaccgtctgggtcgggagaccggcctggacactcgtggcgaaagcctcgagg |
| | | tacggtaacactcctcccgctaggggctatgggcagccaagctctaagtcgctctcgcagcgatacgagtgcagtccacagac |
| | | tagatggcagtgggtatcgctcatcgaagcgatgcttaagacatagtcggtccccaccgagaggtggttcgacggaggatcga |
| | | ggcgaccgcctcgtgagagccgccgaggtcgggcgaggtctagctagacgacccgccgacggagcacacgagtaattctaga |
| | | gc |
| 3086 | Delitschia_ | gaacgaaatttaggggatcgaaaacaattttacagttaaacgtgacagtttctggtcgtcgaaagtgcgtaagcactaatctg |
| | didyma | ccccccccgggggcgggcgacatcatcaaattgcggggacccctgggaaccccagctaccacacacggttgaaaggcactgtg |
| | | ccaacttcggagagagaccgaantgaagtaaaaacgctgggtcaggatctattcctacaggaaatccgcagccgacgccctaa |
| | | accccctcccgggggcatgggtgcacttcacagactaaattgtgatgggtarggggcaytscctcctgcttaagatttattcg |
| | | ggcccggtgcgaaarcgstngnaacaatcttaanaatycgncagataccgtcgtagtcttaaccgtaaactatg |
| 3087 | Coelastrella_ | gaattgacggaagggcaccaccaggcgttaataagagctgctcagctttagcgccagtggcttggaaacaagctgctagtcac |
| | multistriata | actcctatttgtgagtcagagacttgctatagcagtgtggcaacatcctcaaattgctgggaagccctaaagctgctacatac |
| | | caaagttggttggaaacaactgactggccaggttaacaacctcgggtatggtgacaactgtagcagatgaagcatgctcacat |
| | | gctgaagtgggttatcagcagccaagtcctaaacgtgctcggtgtgttcaactctaccgcaaggtagcttattccaagacaca |
| | | ccaagcatgcatggatgcagttcacagactaaatgggggtgggccctcgaagcttaagatataggagcctgcggcttaatttg |
| | | actcaacacgggaaaacttaccaggtccag |
| 3088 | Naohidea_ | gtctaggttcgggggggagtatggtcaagcgttgcggccatgaagagtgacaaggtaacttgttgctagtctatttaataggc |
| | sebacea | aacaccgtgcaaattgcggcaacacccttaagcttgttaacaccaagcacggtaccgaaaggtacgtgtggccagatgtagtg |
| | | atctgggtatggttatagtttatcangtttgggcaagcacgcagccaagttcttactcgttagcgagagaatgcagttcacag |
| | | actaaatgccggtgggtaggaaaaagcaatttcattgcaaaagtgttcttatcaaatcctacttaagatatagtcgggccacg |
| | | tcgaaagatagtggattagcctgcgcaaggctgaaacttaaaggaattgacggaagggcaccaccag |
| 3089 | Sclerotinia_ | gaaacttaaagaaattgacggaaaggcaccaccaggcgttaactgcagtaactctgcgcctaaaagcagctcgtaagagttgg |
| | trifoliorum | tggtagccttcaaatatgctagttggaaatcagctataccttcaaactgcggggaactccttaaaaactcaactactaaacct |
| | | caattgaaagattgtggtggccagctgattctgggtagagtaataatgttgagaatttggacaatccgcatccaaccctctaa |
| | | ggttttaaactacgaggaaggttcagagactaaatgtaggtaggtagcgtcgctacttaagatatagtccgcctcgagattaa |
| | | cgtctcaagaataacaatgggagcctgcggcttaatttgactcaacacggggaaactcaccagg |
| 3090 | Pseudalle | ggaagggcaccaccaggggttaacgtctaactgttaagccacagaagaaatctgctcctgaaagccgcccgaaagggtgagg |
| | scheria_ | tggtgtccggagcgtgcgagctcgaatttaaaggctagtcctccaatgcggagggcgacatcctcaaattgnggggaactcct |
| | boydii | aaagctttcactccaaagccggttcgcgaaagccgccggtggccaggttaacagcctcgggtactggaaaaacgtggaagatg |
| | | cgacaatggacaatccgnagccaagcccctaaaggntccccgtcgagggacgcccacggggaaggttcagagacttaacggg |
| | | gatgggtatgcgccgagaggcgcctgnttaagataaagtccggccctgcncgagagcgcgggggtagcagggaacctncg |
| | | gcttaatttg |
| 3091 | Sporobolo- | acttaaaggaattgacggaagggcaccaccaggagttaacgcaagacaaagctctgctcttcaaggcgaaccagaaatggttt |
| | myces_ | gtggtgtcccttgtaacattcgccagtactctacgcgagggcgacactttcaaattgcggggaactcctgagagccctcagct |
| | sasicola | accaagtcgaagcgctgaaaagcctttgatggccagattaaaaatcttgggtacggtaacaacgttgagggattggatgatcc |
| | | gcagccgcctccccttcaatggggcgcggttcagagactaaacggaagtgggtccactctaaggatctaagatatagtccatg |
| | | gcttgaggaaactcaaag |
| 3092 | Nigrosabulum_ | ggctgaaacttaaagaaattgacggaagggcaccaccaggggtaaaatccgcagaagctctgcccctaagagcagccggga |
| | globosum | gaccggtcggtgggagaaaacagcccgggcgccgcagagcgcgaaccaactgctagtcggcccccccgggggtccggcg |
| | | acatcctcaaattgcggggaatccctaaagccgcgtgctaccaagtgggccgccgaaaggccgcccgtggccgggtctaaca |
| | | accccgggtacgttacaacgcaccggatgacaacaatgggtgacctcgcagccaagctcctacacggcttgccgcgcaggag |
| | | gaaggtccagagacttgacggggatgggtcccgccgccagccggcgtggcctaagataaagtccgtctgcgcgcgaaagcg |
| | | ctgcagagccctaaacgggacgtgcggcttaatttgactcaac |
| 3093 | Beauveria_ | attgacggaagggcaccaccaggggtaaaccgcttcatgcagcccgcagtagctctgctcccaaaagcagcccgaaagggtc |
| | bassiana | aagtggtgttcctgtccgcgctccggcgcggcgaaccaaagtgctagtctctccggaggcgacaccctcaaattggggaaac |
| | | ttctaaaaccagtgtcaccaagccggcgtcgaaaaggcgtcggtggccggggcaatgacctagggtacggtaaaagcacact |
| | | ggatatatggacaatccgcagccaagcctcgtcacggggaaggttcagagactaaatggggggggtagtgtgctggaggatt |
| | | cgtcctccgagcgctcgctcacacactgcttaagatatagtccgcccgcgctggaaacagcgcggattggcaagctcaaataa |
| | | acgggagcctgcggcttaatttgactc |
| 3094 | Mycoarachis_ | ggctgaaacttaaagaaattgacggaagggcaccaccaggggtaaaacgccgcaagctctgctccggaaagcagccaggag |
| | inversa | actggtcggtggtgccttcagaacacaagtgctagtcaccctccgcggtggcgacaccctcaaattgcgggaaatccctaaag |
| | | ccgcgtgctaccaaccggaccgccgaaaggcggccggggctgggctaactgccacaggtatggtcacaacgcaccggatga |
| | | gcggccgagaggccgcgaagtgggtgatccgcagccaagctcctacgtagaggtcccctctatacggagaaggtccagaga |
| | | cttgacggggggggtgcggcagcggccgcgccgaagataaagtccgtccggcgtgcgaaagcgccccggatccctaaac |
| | | gggagcctgcggcttaatttgactcaacacggggaatctca |
| 3095 | uncultured_ | ggaagggcaccaccaggagttcgccgaactgcgatttattagcagactctgctcctgaaagccgcccgtagaagggtcggtgg |
| | soil_fungus | tcgtccaggcttcgagccgaacaagtgctagtcacgcgagtggcaacatcgcccaactgcgggaacgtcctaaagctctacct |
| | | accaacccgccgtcgaaaggcgtcgggggcccggttaatgcccggggtacggtcacaacgggagagatatacaatggataatc |
| | | cgcaaccaagcccctacagcaatctgccacggggaaggtccagagactaaacggcggtgggctcacggggagcttaagatat |
| | | antccgtccggggatcagcgtcccacgga |
| 3096 | Kirschstei | gaaattgacggaagggcaccaccaggcgttgtggcagactctgcgccgagatgcggccccccaggcggggggtgggtggt |
| | niothelia_ | gccgtccttaaatgccagtctccgacgagcctcatcgtcggggggacatgccgaaattgcggggacatcccaaggctccgac |
| | aethiops | caccaggtcgccccccgaaagggggcgacgaccgggttctccccgggtagggtgggcacggtagagctggggatcactc |
| | | gcagccgagcgccttccgttccatcggggccccgagggcccggggggaccacggcgccgggtccagagactagacaggc |
| | | atggggggccccgttgcggaggggcccgcttaagatatagtccacgagcatccgaggatgcctccatcagggagcctgcgg |
| | | cttaatttgactcaacacg |
| 3097 | Protomyces_ | tatggtcgcaaggctgaaacttaaagaaattgacggaagggcaccaccaggagtaaacgtttatgtcgcagttaactctgctc |
| | lactucae | cgaaaagccactcgaaggagtgtggtgtccttagaggctagtcagtacgctggcaacacaatcaaattgcgggaaactcctta |
| | | gagctcaagctaccgctgctgcccagagatgggttgtggcaccaggttaacgacctcgggtatggtaacaacgcttgagattg |
| | | gacaatccgcagccaagctccctcacgggaagaaggttcagagactagatggttgtgggttgaacacacgtttattgaaacat |
| | | gttcggcttaagatatagtccaatccagtgtggaaacgcactggtctaaaagatagaaaatgggagcctgcggcttaatttga |
| | | ctaacacggggaaactcaccaggt |
| 3098 | Bensingtonia_ | accaggagttaacaaacaggctctgctccaaacagtagctcgaatagagtacggtggtcgctgtcgtttaaatactagtctgt |
| | ciliata | ttataacaggcaagatcttcaaattgcggggaagtactggggctctaatctaccaagtcattgtctgaaaaggcatgatggcc |
| | | tgaggtaataactcaggatatggttacaacgattagagatgttacaatgtatgattcgcagcccaatccctcaatatgggatc |
| | | aggttcagagactaaacggagatcgatctaggcattgaactaggtttaagatatagtccattattctgcttaacagcaattga |
| | | atgtattaattgggagcctgcggcttaa |
| 3099 | Pyxidiophora_ | acaccaggggtaaacttcgaactgtagccgcagtcgctctgctcctaaaagcagccgggaaaccgtcaggtggtaagctcctt |
| | arvernensis | gcccaggtgggcgaagtattgctagtcgtcgtaagggcaacaccctcaaattgacgggaagtccctaaagccagctgagacca |
| | | agcgagtatccgaaaggtgctcgtggccgagctaatagccctgggtatggtaaaacagcagctggatattacaatgggtaatc |
| | | cgcagccaagctcctaaacagatttatactgcacggagaaggttcagagacttgacggggggggtgcgtaattggtttttatt |
| | | gaactgcttacacgtgcttaagataaagtccgttgctgctgtaagcaggacaggtcttggtaacctttgaccattaaagggag |
| | | tgcggctt |
| 3100 | Geomyces_ | cttaaagaaattgacggaagggcaccaccaggagtaaacttagtttcgtcgcagcaaactctgctccagaaagcagcccgaaa |
| | pannorum_ | gggttagtggtgtcccaggcctcgggccgaattaaatgctagtctgccacggcaggcgacaccctcaaactgcggggaactcc |
| | var._ | taaagcttctaggtaccaagccggcgtgcgaaagctgccggcggccagggtaattgcctcgggtaccgccaaaaccctagaa |
| | pannorum | gatgctacaatggacaatccgcattcaagcccctaagggaccacggtcctacggggaaggttcagagacttgatgggggggg |
| | | tttgagaatactcaggcttaaggtaaagtccggccgctggagaaatccatgcggattggtaaataaaacgggagctgcggctt |
| | | aatttgactcaacacgggaaactcaccaggtcca |
| 3101 | Peni- | tcgcaaggctgaaacttaaagaaattgacggaagggcaccacaaggcgtaaacttttaccgctagccgcagtaactctgcgcc |
| | cilliopsis_ | gaacagcagcccgaaagggtgatgtggtgaccctctaaacagtaaatgctagtccagcacggctgggcgacacgctcaaattg |
| | clavarii- | cgggaaaccccttagagcctctagctaccaagccgccggtcgaaagacggcggtggccaggttaattccctcgggtacggtga |
| | formis | taacgctagaggattgggcaatccgcagccaagcccctacgttccgcagggaatacggggaaggttcagagactaaatgggtg |
| | | tgggttgggtcctccgggggaggacaccagcttaagatatagtccggcgccaggggaaaccatggcggttcaggtatacctgt |
| | | cttaacgggagcctgcggcttaatttgactcaa |
| 3102 | Dactylella_ | cttaaaggaattgacggaagggcaccaccagatgtaaaccgtagttaactctgcatctgaaagccgtctcgggagtagcatcc |
| | cylindrospora | tgaaggttgaatctggccctgttcgcaatggctagtccaagctttttcagctgggcaacaccctcaaattgcgggaaactcct |
| | | aaagctgctgaacaccaaacctgaccggtaacggttgggtggccgccctgaaagggtgcgggtatggtaacagagtcagcaga |
| | | ttgctcgccagcgggccagcaaatggacgatccgcagccaagcgaccggttcaaacccaaccggttgaaggttcagagactag |
| | | atgggggtgggttttcaaaaacttgaaggcttaaggtatagtccagccgttccagcgaaagcaaacgggtcaggcaatgggag |
| | | cctgcggcttaatttgactcaacacggggaaac |
| 3103 | Graphium_ | ggaagggcaccaccaggggtaaactgaagccgtatcgctctgccccgaaaagcgagcccgtaacgggttcggtggttgccg |
| | basitruncatum | gcattcgctaggtccccgtccaggcgggggcgacaccctcaacgtgcggggaaccctcgaagggcaccactcctaagtaacg |
| | | gtcgaaagacgcgctatggcggcgccgtaatggccgccggtacagggaaaacgtggtgcttaggaggcgattcgcagccaa |
| | | gctcctaccccgcctccggggcgcagggagaaggtccagagacttgacgggggggggtgngcgcgcagcccgcgccnn |
| | | ngngcctaagataaagtccgtgcctgcgcgagagcgcgggaatgctctacagcagagccccgaacaggggatgcggcttaa |
| | | tttgactcaacac |
| 3104 | Graphium_ | ggaagggcaccaccaggggtaaactgcaaagccgtatcgctctgccccggaaagcgagcccgtaacgggtccagtggtcgc |
| | peni- | cggcattcgctaagtctgcacgcgcagcggcgacaccctcaaattgcggggaaccctcgaagggcgtcactcctaaggacgg |
| | cillioides | tcgaaaggcgcgctatggngcagcgtaacggctgcnntacagggacaacgtgancctaggaggcgattcgaagccaagctc |
| | | ctaccngcgccccggcgccgtagggagaaggtccagagacttgacggggggggtaggtcctcgcgggcccgcctaagata |
| | | aagtccgtgcctncgcgagagcgcgggaatgctccaagcagagccccggaaaggggctgcggcttaatttgactcaacacgg |
| | | gg |
| 3105 | Scotie- | ntcaacacgggaaaacttaccaggtccagacatagtgtaacgatagctgctcagctttagcgccagtggcttggaaacaagct |
| | llopsis_ | gctagtcacactcccatctctaggcaagacagcctgcagtatggatgtggcaacaccctcaaattgctgggaagccctaaagc |
| | oocystiformis | tgctacataccaaagcttgttggaaacaactagctggccaggttaactacctcgggtatggtgacaattgtcccagacatagg |
| | | gaggattgacaagattgagagntntttctng |
| 3106 | Bensingtonia_ | gcgttgattacgtccctgcccttttttcattttaaacgcttgatgtacaaggggttcaaagtgtcttggtaaaagatgctagt |
| | thailandica | agtgaacacggtattactatttgtcaacttgccagaccgtcaaattgctggaaagcctaaagctttaactaccaagtcaagac |
| | | agaaatgtactgatggctgggtcacgacccagggtaaggtgaaaacgttaaagatgtttaacaatgggtaatcagcagccaag |
| | | ccctaaggtgtcttgtacactagggtgagttnacagactagatgtcggtcggcttttcgattaatcgaaaaagcttaagatat |
| | | agtcggcccaagtctgaaaggactttggataaagtgttgtacacaccgcccgtcg |
| 3107 | Gonatozygon_ | accttatcatttagaggaaggagaagtcgtaacaaggttttgcctcgagcaaacgcttgcggaagccttagcagcccgaaaag |
| | aculeatum | gttcccctcgcgaccaaatccgaaggaaggggattgaatgctagtgcccaagttgccaacttgggcgcgacatcgccaaattg |
| | | cggagaactccactatttcgcctcgattaccaagcggtacgccgaaaggactccgtggccgggctagttgcctcgggtaaggt |
| | | cataatatcgaggatgagggaagatccgcagccaagccctcgcgccaaacgctcgtgggtgctgttcacagactaaacggcgg |
| | | tgggttccggactctagaagcctggagcctaagatatagtcgttcgcctcgaaagagcaatctgcagagattcaccgttccgt |
| | | aggtgaacctgcagaaggatca |
| 3108 | Mesotaenium_ | aacaaggtttggctcgatcgtggatcctttcgcggttgaaactaccgaagccttagctgcccgaaagggtgacctccgcgact |
| | caldariorum | cgcaaaacaaggagaggtctcaaaagctagtggctcctagctgctgctcgtgcagtgagtgcgacatcaccaaattgcgggaa |
| | | atccctaaagctttgactaccaagtgcggcgccgaaaggctcgcatggccagggtaatgacctcgggtatggtaacaacgtca |
| | | aagatgcaacaatgggctatccgcagccaagtcctcgccgcttcgcagcggttggatgctgttcacagactagatggtggtgg |
| | | gttctcggatgcgtcccggggcttaagatatagtcgggcctgcttggaaacagcgggaaatcgcagatgcattcaccgttccg |
| | | taggtgaacctgcagaaggatcaa |
| 3109 | Statrastrum_ | aacaaggttccgtttatgatccttctcgccccgttatgggttcgagtcgcacggaagccttagcactatgtgcattgtatttt |
| | sp._M | aatacgctgcacccgtgctagtgccaatgatgatgtcattggtgcgacgtcatcaaactgcgggaaagctcctatatggcatg |
| | 752 | ggataccaagcacggcatcacacattgtgtgtgatgagtgtggctgagttaatgcactcgggtaaggtaacaacccccatgtc |
| | | aagaggaccgacctgcagccaagcctttggttataccaagggtgctgttcacagactaaatgatgacgggttgctaatcacaa |
| | | agcgtgattgcagctcaagatatagtcggactcactctagagtgagacaacacgtaataccttcccgcacgtgaattcaccg |
| 3110 | Cylindroc- | tttagaggaaggagaagtcgtaacaaggtttcattgcgtccttctgcaagtttgctactgaagccttagctgcctgaaaaggt |
| | ystis_ | gaccttgacgacttgataaagaaacaaaggtcttaaatgctagtgttcaccctgagtggacgcgacatcaccaaattgcggga |
| | ibrebssonii | aaaccctaaagctttgactaccaagtacacgtccgaaaggagtgcatggccagggtaatgacctcgggtatggtaataacgtc |
| | | aaagatgaaacaatgggtaatccgcagccaagtccttgcttgtcatggatgccgttcacagactaaatggtggtgggtttgcg |
| | | aagcaagcttaagatatattcgggcctctcggaaaccaaaagggggcagtttcnccgttccgtaggtgaacctgcagaaggat |
| | | ca |
| 3111 | Closterium_ | gaggaaggagaagtcgtaacaaggttgcccctgggcacgatccttccgaggaccccagacggaagcctgagctatctctgtcc |
| | spinosporum_ | gcggcgtggagagagagagagctaggtgtggctcatgccataccgacaccgtccaactgcgcgagagttccagaccctggcc |
| | var._ | ggtaccaagcgctctcaagagcgtggccggagaagctgtcatccatcgcggctctcttcgggtatggtcataacccggcaccg |
| | spinosporum | ggctcaaggataatccgcagccaacccgtctcaggcgggaccgttcacagactgaacgacggtgggtccatgcgtgcgtggg |
| | | cttaagatacagtcggcatctccccatttaagggagattcgagcaagcacagtcggcctgcagcgatttcaccgttccgtagg |
| | | tgaacctgcggaaggatcattg |
| 3112 | Closterium_ | gaggaaggagaagtcgtaacaaggttgcccctgggcacttccttccgacaagggacccagacggaagcctgagcaccgccc |
| | spinosporum_ | gctggcgcacagcgggctcggtgctaggcggtgcgcgcgtgcgcgcactgtcgacaccgtccaactgcgagagagttcccg |
| | var._ | accgcggatggtaccaagcgcctcagagcgtggccggaggcggagcgccgctcccccctcygggtatggtcacaacccatc |
| | malaysiense | cctggcagacggatgatccgcagccaacccgtcgcgtcgtgacgggccagttcacagactgaacgacggtgggtccgggtgt |
| | | cgcaagccgcgaaacccgggcttaagatacagtcggcggctccgggagaaccggagtcccaccccctcccctcgcagagga |
| | | cttcaccgttccgtaggtgaacctgcggaaggatcattg |
| 3113 | Phialophora_ | ggtcatttagaggaagtaaaagtcgtaacaaggtttcaccacggagaccatgcatcccctgagggaggcagcgcgactgtaaa |
| | gregata_ | taacgctgccgtatgcaagtcaggctaccgctctggcaacacgatcgaattgcggggacctcctaaagcttcactcactacct |
| | f._sp._ | acccccgaaaggaggcagcacagttataacagtgaagatgttacaatggacgatccgcagcggagaccctacagcgcaagcca |
| | adzukicola | cgggtagcgttcacagactaagtgattgtgggtagtacggtatactacttaagatatagtcgaacccttactgaaagtgaggg |
| | | gtaaaggtccgtaggtgaacctgcgga |
| 3114 | Dactylella_ | gtcgtaacaaggtttgaagtaagtttccttccataggtgacgctcacgagaagcctttgtggccccgcaaggggtacctgcca |
| | gampsospora | cgactgtaaagaatggatgcaggtattaaatcgcaagtcagctagcagctggcaacacttttgaattgacggggacaccctaa |
| | | agcccacctctaccaaccagctggagaaatccggttggggcctgtgttaacagcacagggtacggtaacaacgaggtcggata |
| | | agccagccctctggcacaatgggtaatcctcagcccaagtccctaatcttttcgttcagaaaaagacatgggaaaggttcaca |
| | | gactaagtggaagtgggttggttcctgaacccaacttaagatatagtcgggctctgtgcgaaagcagagaggagtcactgttc |
| | | cgtaggtgaacctgcggaaggatcatta |
| 3115 | anamorphic_ | gaggaagtaaaagtcgtaacaaggttttgctaagcaacagtccttctgtagcagagcttaacggagaccttgcgccaaaaggc |
| | fungus | gactataaataatcggcagcaagtcagtatcactggcgacacaatcgaattgcggggacgctttaaagctcgacggtaccaac |
| | JCM_80 | cgctggaaatcccagcggggctagcatgctagaggtcagaactcgtcgagatgtacaataagcaatccgcagcggtccccttc |
| | 69 | gaggggccgtccacagactaagtggttgtgggtaggacgtctgttctatctaagatatagtcgggccttgcgagagatcgcag |
| | | gggtttctgcgtccgta |
| 3116 | Encoelia_ | gcgtataggggcgaaagactaatcgaaccattggaagctactaggtggttaagaagagacttgaacaagtctcctagtcctat |
| | fascicularis | tgtagggcaacactatcaaattgatcgggaactacctgttaaattctatgctaccgcagcctggtcgaaagacgggttgcgca |
| | | ccaggttaacgaccttggggatggtaataatgcttagatagggtatatccgcagcgaaatcctaaggctcaggctatggatag |
| | | cgttcacagactagacgatagtgggcttggcagctctatcttgccacagcttaagatatagtcgaacccttcagagatgcggg |
| | | gcgtgagtgatggatgcaagtctgttgctcagcgcatcagatgctagtagctggttcctgccgaagtttccctcagga |
| 3117 | Grovesinia_ | gcgtataggggcgaaagactaatcgaatcatccacatggcatatatttatatagttgctaggtgattaagaagaggcttgaac |
| | pyramidalis | aaggtctcctagtcctaccttagggcaacactatcaaattgttcgggaactacctgttagaattctaagctaccgcagctttg |
| | | tcgaaagacggttgcgcaccaggttaatgacctcggggatggtaataatgcttagatagggtatatccgcagcgaaatcctaa |
| | | ggcttctataagctatggatagcgttcacagactagacgatagtggggcttttatgatattgaatcattagggcttaagatat |
| | | agtcgaaaccttcagagatgaagggcatgagcaataggatatgaatacaatccgaatgctcatgcattacatgctagtagctg |
| | | gttcctgccgaagtttccctcaggaatagttgctaggtgattaagaagaggcttgaacaaggtctcctagtcctaccttaggg |
| | | caacactatcaaattgttcgggaactacctgttagaattctaagctaccgcagctttgtcgaaagacggttgcgcaccaggtt |
| | | aatgacctcggggatggtaataatgcttagatagggtatatccgcagcgaaatcctaaggcttctataagctatggatagcgt |
| | | tcacagactagacgatagtggggcttttatgatattgaatcattagggcttaagatatagtcgaaaccttcagagatgaaggg |
| | | catgagcaataggatatgaatacaatccgaatgctcatgcattacatgctagtagctggttcctgccgaagtttccctcagga |
| 3118 | Myrioscle- | gcgtaaaggggcgaaagactaatcgaatcattaacactactaggtgattaagaagaaacttgaacaagtttcctagtcctatt |
| | rotinia_ | tcagggcaacactatcaaattgatcgggaactacctgctgaattctaagctaccgcagccttgtctaaagacgggtgcgcacc |
| | curreyana | aggttaataacctcggggatggtaataatgcttagatagggtacatccgcagcgaaatcctaaggcctttgctatggatagcg |
| | | ttcacagactagacgatagtgggcttctcagtgagagcttaagatatagtcgaaaccttcagagatggagggcgtgagtatct |
| | | tcgaatgctcatgcatcaaatgctagtagctggttcctgccgaagtttccctcagga |
| 3119 | Myrioscle- | gcgtaaaggggcgaaagactaatcgaaccattaaggaatagaccaagctctaggtggttgagaaacccctttggtattagtcc |
| | rotinia_ | tggagacagggcgacattgtcaaattgttcggggaccacccgttaaattacatactaccgcaacaggcctgaaaggcctgcga |
| | ciborium | gcactaggggtagcgcctctagggatggtaataacgtatgtatagggtatatccgcagcgaagttctaaggcctcacggctac |
| | | gaatcgcgttcacagactagacggcaatgggctcttcacagggcttaagatatagtcgaacccctcagagatgaggatggaat |
| | | caatgctagtagctggttcctgccgaagtttccctcaggagcgtataggggcgaaagactaatcgaaccattaaggaatagac |
| | | caagctc |
| 3120 | Myrioscle- | gcgtataggggcgaaagactaatcgaaccattaaggaatagaccaagctctaggtggttgagaaacccctttggtattagtcc |
| | rotinia_ | tggagacagggcgacattgtcaaattgttcggggaccacctgttaaattacatactaccgcaacagcgttgaaaggcctgtga |
| | luztlae | gcactagaggtaacgcctttagggatggtaataacgtatgtatagggtatatccgcagcgaagttctaaggtcttacggctac |
| | | gaatcgcgttcacagactagacggcaatgggctcttcacagggcttaagatatagtcgaacccctcagagatgaggatgggat |
| | | caatgctagtagctggttcctgccgaagtttccctcagga |
| 3121 | Myrioscle- | gcgtataggggcgaaagactaatcgaaccattaaggaatagaccaagctctaggtggttgagaaacccccatggtattagtcc |
| | rotinia_ | tggagacagggcgacattgtcaaattgttcggggaccacctgtcaagttacacactaccgcaacagtgctgaaaagctgtgag |
| | caricisampu- | cactagagatagcgcctctagggatggtaataacgtgtgtatagggtatatccgcagcgaagttctaaggcctcgcggctacg |
| | llacae | aatcgcgttcacagactagacggcaatgggtcctcgcagggcttaagatatagtcgaacccctcagagatgaggatggaatca |
| | | atgctagtagctggttcctgccgaagtttccctcagga |
| 3122 | Botrytis_ | gcgtataggggcgaaagactaatcgaaccattaaggaatagaccaagctctaggtggttgagaaacccccttggtattagtcc |
| | tulipae | tggagacagggcgacattgtcaaattgttcggggaccacctgttaaattacatactaccgcagcagtgctgaaaggcctgtga |
| | | gcactagaggtaacgcctctagggatggtaataacgtatgtatagggtatatccgcagcgaagttctaaggcctcacggctac |
| | | gaatcgcgttcacagactagacggcaatgggctcctaacggggcttaagatatagtcgaacccctcagagatgaggatggaat |
| | | caaagctagtagctggttcctgccgaagtttccctcagga |
| 3123 | Botrytis_ | gcgtataggggcgaaagactaatcgaaccattaaggaatagaccaagctctaggtggttgagaaacccctttggtattagtcc |
| | porri | tggagacagggcgacattgtcaaattgttcggggaccacctgttaaattacacgctaccgcaacagtgctgaaaggcctgtga |
| | | gcactagaggtaacgcctctagggatggtaataacgcgtgtatagggtatatccgcagcgaagttctaaggccttcacggcta |
| | | cgaatcgcgttcacagactagacggcaatgggctcctcgcggggcttaagatatagtcgaacccctcagagatgaggatggaa |
| | | tcaaagctagtagctggttcctgccgaagtttccctcagga |
| 3124 | Sclerotinia_ | gcgtataggggcgaaagactaatcgaatcattaaggaatagaccaagctctaggtgattgagaaacccctttggtattagtcc |
| | sclerotiorum | tggagacagggcgacattgtcaaattgttcggggaccacctgttaaattatatgctactgcagcagtgctgaaaggcctgtga |
| | | gcactgagggtaacgccctcagggatggtaataacgcatatatagggtatatccgcagcgaagttctaaggcttcgagctatg |
| | | aatcgcgttcacagactagacggcaatgggctcctcgcggggcttaagatatagtcgaacccctcagagatgaggatggaatc |
| | | aatgctagtagctggttcctgccgaagtttccctcagga |
| 3125 | Sclerotinia_ | gcgtataggggcgaaagactaatcgaaccattaaggaatagaccaagctctaggtggttgagaaacccctttgtattagtcct |
| | tetraspora | ggaaacagggcgacattgtcaaattgttcggggaccacccgctaaattacatgctaccgcagcagtgctgaaaggcctgtgag |
| | | cactagaggtaacgcctctagggatggtaataacgcgtgtatagggtatatccgcagcgaagttctaaggccttctgctacga |
| | | atcgcgttcacagactagacggcaatgggctccttgcggggcttaagatatagtcgaacccctcagagatgaggatggaatca |
| | | atgctagtagctggttcctgccgaagtttccctcagga |
| 3126 | Anabanea_ | aaataattgagccttagagaagaaattctttaagtggatgctctcaaactcagggaaacctaaatctagctatagacaaggca |
| | sp | atcctgagccaagctgaagtagtaattagtaagttaacaacagataacttacagctaatcggaaggtgcagagactcgacggg |
| | | agctaccctaacgtcaagacgagggtaaagagagagtccaattctcaaagccaataggcagtagcgaaacgtgcggagaatg |
| 3127 | Nostoc_ | agataactgagccttgaaggagaaatccctcaagtggaagctctcaaactcagggaaacctaaatctggtgacagacatggca |
| | sp._PCC7 | atcctgagccaagcccgaaaattttagatttgcgattttagatttgggattagttttcaatctaaaatccaaaacccaaaatt |
| | 3102. | gagggaaggtgcagagacccgacgggagctaccctaacgtaaagtcgagggtaaagggagagtccaattctcaaaatctgatt |
| | | tggctattgccatcaggtagcagtgaaaactgcgggaggatg |
| 3128 | Chloroplast_ | aattggattgagccttggtatggaaacctactaagtgataattttcaaattcagagaaaccctggaattaataaaaagggcaa |
| | Gentiana_ | tcctgagccaaatcctatttttcgaaaaagcaaaaagaaaggcttagaaagaaataaaggataggtgcagagactcaacggaa |
| | ligustica | gctgttctaacaaatggagttggttgcgttggtaaagaaatctttctaccaaaaattccaaaaggatgaagaagaggggtgac |
| | | tgtatcaaatgattcgcccacagcctgtagatcaacggattaatcggacgagaataaagagagagtcccattctgcatgtcaa |
| | | tgccgacaacaatgaaattgatagtaagagg |
| 3129 | Chloroplast_ | atcggtagacgctacggacttaattggattgagccttggtatggaaacctactaagtgataactttcaaattcagagaaaccc |
| | Draba_ | gggaattaacaatgggcaatcctgagccaaatcctggtttacgcgaacaaaccaaagtgtagaaagcgagaaagaagggatag |
| | tomentosa | gtgcagagactcaatggaagctgttctaacaaatggagttcactaccttgtattgatcaaatgattcacttcatagtctgata |
| | | gatccttggtggaactgattaatcggacgagaataaagatagagtcccattctacatgtcaatactgacaacaatgaaattta |
| | | tagtaagatgaaaatccgttgacttttaaaatcgtgagggttcaagtccctctatccccccaaacagtctgtttg |
| 3130 | Chloroplast_ | atcggtagacgctacggacttaattggattgagccttggtatggaaacctactaagtgataactttcaaattcagagaaaccc |
| | Draba_ | gggaattaacaatgggcaatcctgagccaaatcctggtttacgcgaacaaaccaaagtgtagaaagcgagaaagaagggatag |
| | dubia | gtgcagagactcaatggaagctgttctaacaaatggagttcactaccttgtattgatcaaatgattcacttcatagtctgata |
| | | gatccttggtggaactgattaatcggacgagaataaagatagagtcccattctacatgtcaatactgacaacaatgaaattta |
| | | tagtaagatgaaaatccgttgacttttaaaatcgtgagggttcaagtccctctatccccatccccaactatactc |
| 2557 | T4 td | GATGTTTTCTTGGGTTAATTGAGGCCTGAGTATAAGGTGACTTATACTTGTAA |
| | | TCTATCTAAACGGGGAACCTCTCTAGTAGACAATCCCGTGCTAAATTGTAGG |
| | | ACTGGTTCTACATAAATGCCTAACGACTATCCCTTTGGGGAGTAGGGTCAAGT |
| | | GACTCGAAACGATAGACAACTTGCTTTAACAAGTTGGAGATATAGTCTGCTC |
| | | TGCATGGTGACATGCAGCTGGATATAATTCCGGGGTAAGATTAACGACCTTA |
| | | TCTGAACATAATGCTACCGTTTAATATT |
| 2557 | Tetrahymena | TAACTATGACTCTCTAAATTGCAAAATTTACCTTTGGAGGGAAAAGTTATCAG |
| | thermophila | GCCTGCACCTGATAGCTAGTCTTTAAACCAATAGATTGCATCGGTTTAATAGG |
| | | CAAGACCGTCAAATTGCGGGAAAAGGGTCAACAGCCGTTCAGTACCAAGTCT |
| | | CAGGGGAAACTTTGAGATGGCCTTGCAAAGGATATGGTAATAAGCTGACGGA |
| | | CAGGGTCCTAACCACGCAGCCAAGTCCTAAGTCAACATTTCGGTGTTGATAT |
| | | GGATGCAGTTCACAGACTAAATGTCGGTCGGGGAAGAATAGGTATTCTTCTC |
| | | ATAAGATATAGTCGGACCTCTCCTTAATGGGAGCTGGCGGATGAAGTGGTGC |
| | | AACACTAGAGCCGCCGGGAACTAATAATAAACATAATATTAGTTTTGGACTA |
| | | ATCGTAAGGTAGCCAAATG |

TABLE B
Group II introns (flanked by 10nt exons)

| SEQ ID | Species | Sequence (10nt exon-intron-10 nt exon) |
|---|---|---|
| 3131 | Paraburkholderia xenovorans | tacgcctatggtgcgacacgaagcatgaggctcgttgttgaagagcgcagtgcatgtccgcttcatcattgtccagtcgcgaac |
| | | atgttcgtacgatagaggacccgctgtgcgaccagaggtggcctaggaaagcatctttcgttggcgacgacgctggatgaag |
| | | cctttccgacaacgtagtccaactgtatgcgccgccgcgaggtgacgtgtagaagctctgagcagcgacggggccgagacg |
| | | caaggctggccggtatgggtaacacatgtgaatcgctgataaacgtcgtcagaagaagaatgccaaagctgctgtcaggcatt |
| | | taccaaacaggtgcacgggcggatgctccgttcataggtcggagcacgtcgtcgagatgcccgccggcgaaaaggcggac |
| | | cctaacccggtcgtgtgatgaagcgggaacgtggtaagcccatacagttgcccacgaggcagacgagtcgcaaggcgagtt |
| | | gttgactgtgcgggtaagagaagacggaaaaagcgaatgctgggctgaaacggtccagataagggtttcaacattaccctac |
| | | gcgaaagcgtgcagacttccgtccggtgtccagcgcaaagctgtcgaaagacttcgtagatggaagcatgaaagagcatcgt |
| | | tcgcggtgtctgcgcccttcgctgcggcggaagaacctcggcaagacaatgcgcaggcgtcagatggtggacgccggtcac |
| | | accggcgtgtgactttggaatgcttgagccgtatgcagggaaacttgcacgtacggttcttaggggaggccgcagcggcgac |
| | | gctgtggccttacccgattttaagcccg |
| 3132 | Methanosarcina acetivorans | gagggtcatggtgtgccacgaggctgcagaaaagatgagggtagacccctcggactcgctttacaggaagttattccaaacc |
| | | acccataagctgctttggtcaaaagccacggtagtgagcattatgggaaaaggcgaggctatgacttcgtcaggtgtgggttaa |
| | | ggagccgaatgaaaatgaaccactgaagacgtatcgaaaacgcgatgatgtcgtcaaaaccaggtggcgtgactaacctgg |
| | | gataagtctggaagatactctgtttactgaccagatggtgaccggtatataggtggcgggaacttaactcgtgcttttctgtggaa |
| | | cgtgggaacctgctcatcgatgtaaagagaaacctccaagcggtggacccgcaaggacaaaagtatcgatgcgatgaacag |
| | | gggcggaacaattcgtagtagtgatgaagtgcctgtaatgggcatggagcgaagggattgtattatccagttttgaactgcggt |
| | | caaccttgtcttcaagggaggaaccaatggacaaaacaaggctttatggctggataatgggagccgtatgaatcgagagattc |
| | | aagtacggatctgagagggcctgggggtgaaattcccctgggctacttaccaatgcccaga |
| 3133 | Thermosynechococcus elongauts | agacaaggcagtgcgacgcgaaagctagccagatgattgtcccactagcccaacaagctagaacgggaccggttgttcccc |
| | | caaccgtagcctaaggaggcatgcgtgactggtaacggtcaggtatgaagccccccgacaatgaagcccgaaccggaag |
| | | gttggagccgaatccgtgaggaggaagcaacttcaccagcgtcaggtgatagggagctaggcttgagggtatggtgaacgc |
| | | aagtgaagtgacgctagaagcctcgttactccaagcaggccaaagatgctgataggcctgagccaaaatggcaaagcggatt |
| | | ggattcactgcttgcttttcagtgaacggggacagcaactccgccggggtatagtcaccacctaacccctcgtgtcatctggttg |
| | | gaacgcggtaagcccgtatcttcgccttgaacattcaaggcaggcaaaccgtaaggaatgctgatgggggtgcgggtagag |
| | | gaggtgggaaaaagcgaatgctagcctgtaatgggctagatagggattgagaatgctggcaggacattcaacatcatcccact |
| | | cgaaagagggcagacttcccgccggtccccccttacgagaaagtctatagaaccttcctaatggtgacaatgcaaatggcggt |
| | | gatcttgagtcactggtgcggtcaccaacctgctcaaaaggtagagaggctgtagatgagtatcgtaaaggttgcatgcacaa |
| | | ccggttcctcttaaatgaggggtcctggggggctcgagccggatgcggggaaacttgcacgtccggttcctagggggctagg |
| | | gggcagcgatgcccccctgctacccgacaatgacggtg |
| 3134 | Bryopsis maxima | aatgcatgacgtgcgacacgaaagtcgctgtatttagtagatatttacaaatcttatcctagcattcggatattcccatactttgggt |
| | | gcaatcctataataataaaagattgtgctaagccttagtaaaagccaaaacttatgatgttctcgtaatataaggcaagggaaaat |
| | | agaatagagttggtttaacggcctcgtaaagatgagaacagcccaaaagggttaactacttcgctaacagactgaatggatcgt |
| | | cgtggtcattcaggaatgcttctaagaagtcggggtgattgtctccaaaactattctatgtatctaaatatagtggaacgtgggaa |
| | | atccaaaaatctccctatgggtaggtgtttagcaataaacactaaaagattttggattgcggattgtcctgttatgtaagaattccat |
| | | gcaaaggaataatgaacccatagcaaaaaggaaaatatgggatgcgaattactcagtaaaaagtaagttctaatacagaaaac |
| | | agcccatatataactggaattcaggaaagttatttccaacaaatgaggacaaggtcttatttcataaaatttatattaaaataacaaa |
| | | gaacaaaggagattgacttatgatagtactttgggagactcaccagagtggataccatccaattggaacaccacaatggcaca |
| | | agttcggaatttaaaatatagaatctttatggcgaaactagaaggtaatcggagaacattacgaagattacaaaaattgatgttag |
| | | atagtcgccacaagatactcctgtccgtgagaagggtaacggtgattaatgatggaaaaatagattcctgggatagataaagtt |
| | | ctaataaaagataaaaatgacagaatggtattggtagaaaaactctataaaatgaatattttacaatacgagcctaagcccgtga |
| | | agagaatacaaataccaaaatcaaacgggaaaactagacctctgggaatccctaccatactagacagaatgtatacaagccat |
| | | agtgctaaatgcattgcaaccagaatgggaagcctgttttgaagcaaatagttacgctttcgcccaggaagaagtgcacacga |
| | | cgcgattggtcgggtgttcaacgtactaagtgttaagtcaaatggaaacaataataaaccatggatattagatgcacatatcgaa |
| | | gggtttttcgataacatatgtcatcaagacatattagaaaaactcgacaacttcccagccaaaaagcttataaaccgttggttaaa |
| | | agcaggctacatggaagaagatatcttttcaaaaactgagaaaggtacaccgaaaggatccattatcagcccactactcgcaa |
| | | acatcgcattgtatgagttagaggaatttcttaaagcatcaccagactcaacaggtcgagtgagggggaatagagtatacgttc |
| | | gatatgcagacgacttcatagtcatatgcgcatcaaaaacggaagctcaacaaaccaagaaacaaatctcaggttggttaaaa |
| | | accaaaggtctcagatttgcgccaaataaaataaaaatcagtgatattgataaaggctttgaatttttaggtgtagatattcgccat |
| | | gtaagtacgaatgcctcgcataaaaccaaaggtaaaatgcttctgatacgcccctctacaaaggctatcgacaaaataacacaa |
| | | aaactgaaaaaagaatggtttctcccgagaagtaaactcatagaaacggtactcacacgtctaaacccaatgatcacgggctg |
| | | ggcaaattaccatcaaaaattttcatccacggatatctttaataaattagacaactggacattccagaaaagttggaattatgcatc |
| | | cagaatgcacgccaacaaatcgaaacattggatttatgataagtattatgggaacttcaatttaaaccgaaaggaccgttggata |
| | | ttcggagataaggaaacaggaagataccttaaaaagtttgcttggttcaatataaagagacacgtggtggtaaaaggctacaatt |
| | | ccccttgtgacccaaggttacaagaatactgggaagaacgccggaaaactctctataaagtcagtagaacctaatcggaacaa |
| | | ataatcgcgaaaaatcaagattatagatgtccagtttgtgaacacccgatgagaatctacagaaacatcacctaatttcccaacg |
| | | aaatataaaaattgaaaattattggaacctagtatatatcatagacaatgccatcaattctgacattccaacccgaaaattgaacga |
| | | gaattacatggaaaattttattcgaggaaaatcgtgttcgtaagcgtctaacaaagaatccgcgaacacacatagaaagattaaa |
| | | actcgaacttttagaacataaggcttgagccgtatgcggggagactcgcacgtacggttcttaggggggaattggtggaaacg |
| | | cctgacctacccgactatttaactg |
| 3135 | Methanosarcinales archaeon | cgttactcacgtgcgccacgaagctgcacaagagatgagggttcggcccctcgaagcctgcttgcaggagccggcttcaaac |
| | | cgccttaagctgctgtggtcaaaagccatggtggtgagcgttaaggaaaaggtcccacaagatgggatcaggtgtagatcatg |
| | | gagacgaacgtaagtgaaccgctgaggaagtgtcgaaagcgtagggacgacgtcaaaaccaggggctggtcgttgacctg |
| | | ggataagtctgggagagcgcctgcttactgcccagatggcgtccggcatgaaggcggcgtgaacatggtcctggctcttgtgt |
| | | ggaacgtgggaacctatcccctaatgttaagggagaaacacaagtgggaggaccccacaagtgtgagagtaccgatgcagg |
| | | ggaataggggcggaccagcccgtattagtgatgaagtttctgtaatggagatagagcgaaggggttgggtcatcctgcttgatt |
| | | taaagatcaaccaggagatgggaggaatctttgatgaaagcaaagccatttaagatttccaagaaagttgtattggaagcgtgg |
| | | aaagaggtgaaagcaaaccgtggagcagcaggtgtagataagaagtcgattgcggattttgagaaggatcttaataataacct |
| | | ttacaagatcaggaatcggatgtcatcggggagctactttcctccaccggtgaggacggtagggataccgaagaagagtggg |
| | | ggagagaggttgttaggcatacccactgtggctgacagagtggcccagacagtggcaaagatgtaccttgagcctttggtaga |
| | | gccatactttcacaaggactcatacgggtacagacctggtaaatcagcgattcaagcagttggagtgacgcgtaagcgttgctg |
| | | gaggtatgattggatgcttgaattcgacatcaaggggttatttgacaatattaatcataacctcttgataagggcggtaaggaaac |
| | | acacaaactgtaaatggatgcttttgtacatagacagatggttgaaggctccatttcagagacaagatggtacgttagtgcagag |
| | | agaaaaaggaactccccaaggtggcgtgataagccccttgcttgccaatctcgttctgcactatgtgtttgataaatggatggag |
| | | cggaactatccgcaagttccattctgccgctatgctgatgatggtgttgtgcactgccggtctgaagccgaggcattaaagctaa |
| | | ggaaaactctgggggctaggtttgggaagtacaatcttgagcttcatccggagaagacgaagatcgtctattgtaaagatgatg |
| | | atcgtcgagatgactatcccaacacgagttttgattttcttggctttacattccggccaaggaggtcaaagaatcggtggggtaa |
| | | gcttttcatcaactttactcctgccgtgagtaataatgcggcagaggcgatgaggcaaaaggctcgaagatggaagatgcacct |
| | | gcgaagtgacaagtccttggacgatctgtcaaggatgttcagtcctattatcagaggctggatcaattactatgggcgcttttata |
| | | aatccgcgctgtacccaactttgcaacatctgaacagaacgttagtcagatgggcaatgaggaaatttaagaggtttagacacc |
| | | atcgtaggcgtgcggaatactggttaggggaaattgctcaaaggcagcccgggctgtttccgcattggcagatgggtgtgaga |
| | | cctacggctggatgatgggagcccggtgagctgagaggttcacgccgggttctgagaggacgtaggggtgagattcccctg |
| | | cgttactcacctgcgccacga |
| 3136 | Marchantia polymorpha | aagagttatcatgacaaaatgcaattacgagcaactactagaccccgagatatttaggctagcttacgagctaaagaaatcgaa |
| | | atcaggcaatatgaaacctggtgcggataaagaaactctcgacggtttctcccaagcctatgttgagaaggtcgtccgtcaact |
| | | aaaagacgaatcatttcaattccgtccgtcacgaagagaattcattcctaaagcagacggcaagcttcgttccctgggcatacc |
| | | atcacctagagacaagatagtacaagaggtcatgaggaggatccttgaacctgtatttgaaccgcgattcctggattcgtctcac |
| | | ggatttagacctcatcgatcaccgcacacggctctacgacaaatccgtcgatggacaggcacctcctggatgatcgaaggag |
| | | acatcaaaggatacttcgacaacatcgatcatcacctactcgcgggattcatagcagagttggtaaaggaccaacggcttctcg |
| | | cgctttattggaaattggtacgcgctggctatgtaaatcaaggcaaagcagagccacacttgctaacaggagtacctcaagga |
| | | aggatactatcgcctctgctttccaacatctacctacaccagttcgatctattcatggaggaaatcaaagtcaaatatacaacgac |
| | | cggtgcgctttccaaaaacaacccgatttacttgaaggcgcggaataaatactacaaacttgtgaaatcattaaaggcttcttccg |
| | | ccgaaatcatccgagcgagacgcgatatgttgaaaatgacttacgggattcaaacaggttctagggtgcgttatgttagatacg |
| | | cggacgattgggtgatcggggtcacgggtccaaaagccctggccgtacaaatcaaagaagaggtctctaccttcctccaaga |
| | | aaaactaaaactttcgcttcaggccgaaaaaacacgtattaccaatctatcaagaagcgaagctttattcctaggaaccttaataa |
| | | gcataacaactcgtaaatacgtgcaaagccagaaggtgggcggggggcacaggcgagcctccctcggtagaatacgcctgt |
| | | gcatccccatcgatatccttatcgggaagctctcacaaatgggggcgtgcgacgaaaagggaacgcccaaagcggtgacca |
| | | aatggatctttctaaacgtgggagaaataatcaacaaatatatggctgtgttccggggatactacaactactactcattcgcagac |
| | | gatatccatcaccttctccaaataatatacatactaagatactcggctatcaacacggtcgcccgtaaactggggcttaacacag |
| | | ccaaagtaataaaacgcttcggcgtggacctaatcttccgggaccacacgaatgagatcaagcataagctcaatttcccgcgat |
| | | ccctacctaataagcgcatgaacttcgccttgagtccgccttcggaccctagagttctatttgatacaagctgcgctcgcattcag |
| | | tgttgaacgacctgtg |
| 3137 | Vibrio harveyi | ttttcttttggtgcgccgagcatggcgttgatctaggaggtgaaagtcctctacgggctcagtcgagcgagaaccgttagcctat |
| | | gcaagggtgttcactgtgaggtgaaatctgaaggaagcaaatggcaaaacttggttttgacgaacagaaatctgatagtaggtc |
| | | actacaacttgggcaacctagccatagatagaatagcccaatgcctcgacgggaagtgtgtagtgataaatcaggcacggcc |
| | | aagggaaagagcaacgtcttacctcgggagatcttattgtttgtctcgatagagactaatgcagcagtgatgttgtgtgacgggc |
| | | aataagaagtcagcagagggcatagtaccttgagacagtacaacttaagggaaggcctgaactgaatatatcaagaagtaagt |
| | | cactagacactcaatcatgtggagtcatagcaagatggacatcatctctacgtcccaatgggcgataccgcaagtaaagctcac |
| | | ggtgacgaagaatggcaagcatggtcggcgtagacaggaggacgagtcttggtgacctcaacttcgttgatggaacaaatct |
| | | gctcctcaaccaacctgaatcaagctttacgaagagtgaagaagaacaagggatgtgcaggtgtcgacaaactcgacataga |
| | | tgcaactatctttaagcttcgacaagcttcaaacgggcaagcgcttcgccagagtcttctggatggaagctaccgacctcaacc |
| | | cgttcttggtgtgggaatccccaaaccgagtggtggcgtcagacaactaggtatcccaacagtgctcgatagaattgtccaaca |
| | | ggcaatcacctcagtactgtcagatatctatgaagctaagttctccaatagcagttacggatttagacccagccgcagtgcacat |
| | | catgcgctagcggcagccagtcgctacatccgagaggggcgaggttatgtagtagatatcgacctagcgaaatacttcgatac |
| | | cgttaatcacgatagactgatgcacagactgtcagaggatatcgcggataaacgagtattgaagctcatcaggtcatatctaca |
| | | ggcaggcataatgcgaaacggtttagtcgaacagagacaacgagggacaccacagggtggtccattatctccgttgctatcc |
| | | aatatcgtattagatgaactggataaagagctagagcgtagagggcataagttctgtcgatatgcagacgactgtcaaatctatg |
| | | ttggtagtgaggaagccgcttatcgagtgaaagagtcgataacggagtacttggagcaaaaactgaaactcacggtaaaccg |
| | | agagaaaagtgcagcaacaagagtgacagagcgaacttacctaagccaccgcttcgggatagatgggacaatccatatatcg |
| | | aagccagcgcaaactcaaatgaagacgcgagtgcggcagataacgaagcgaaaccgaggtcgagagttaaaggtgataat |
| | | cgccgagttaactcaatacctaagaggttggcaagactacttcaaactcgccatacggaagagcagcatgaagcgcttggac |
| | | gaatggatacggcgacggctaagatgctatcgacttaagcaacgcaaacacagatacggcatagcgacatggttacagcga |
| | | caaggtgtaacagagcgtaatgcgtggaagttagcgatgtcagataaaggttggtggcggctagcattaacgccccagacaa |
| | | atcacgctatgccaacaaaatggttcgaggagttgggtctgtactcgttgcgagatgggtatgagtcactaaaagtatattcgga |
| | | accgccgtatgcgacccacgcttgtacggtggtgtgagaggacggaggccgtgaggcctcctcctactcgattattgccttt |
| 3138 | Crocosphaera watsonii | ttaggggatagtgcgattcgttgagctagaaagctgacatccaagtatattacaaggatacagcgtgtagctcgttcaagagcct |
| | | tttaaaaggtaatccaaaggttaaatgaccaatcaccctccgaagaatcaccctccgaagagaaaacaaagcctagagcgcat |
| | | tctttaaaaggggtaaatacgaaggtcgaatgggtaaaacaacacccaacaaagcaataactaataaagtttacataacttaatt |
| | | agttattaccgaactagctaagtagggataaccgaacagccaatgaacctggaaacttgaataactgttggtttgttggcagaaa |
| | | ggagccacactcggaatcaagggataactcatcccaagagtgacgacgtaaccctccgttttggaattagtaaacccaggatg |
| | | gaagcagggttaaaaggagaacccgtcatgaaacagcatttcggagtcgttctttaagtcaatcggtcatggttaaacattacgt |
| | | tgaatcatcgtcctgaaccctcatgaatactataagtagccaaatctgtttgataggctgcgttcaaaagaacaaggtgaaaagt |
| | | ggagagtttgtcccctcatccacacagattcctaaaaataacctgggggagagatgaaacctaaaaccgatatacccaaatca |
| | | acggaacgaattaagtccatgatttctcttaccatcaggtcgatgatgtaagtagtgacaaacttaagcgcaagggtcatggatg |
| | | tgagaagcaataagaagcaaatgcctacttgtaatgagtacggataagctgacgtattgctggttaattagaagaaatagtctct |
| | | attagaggtagttaaatgtcgaatgcgagtataaccaagactacgccagaatggaacacaatcaactgggcaaaagtccaga |
| | | ggaaagtgtttaagcttcaaaaaagaatatttcaagctgttaaatcaggtaataaagttaaagctaaaaagcttcagaagttactgt |
| | | taaagtcacactacgcaaagctcctagccatacgcaaagtgacccaagacaaccaaggcaagaaaactgctggagttgatgg |
| | | gataaaggcattacgccccaaccaaagactaaagctaattaaagaacttaccttaaaaggttacaaagcaaaggcacttcgac |
| | | gggtttggattcctaagcctggaagagaagaaaaacgtggactgggaataccaaccatgaaagacagagcgatgcaagcctt |
| | | ggttaaatcagccttagaaccatattgggaagcccaattcgagggaacatcttatggtttccgtccaggcagaagcgcacatga |
| | | cgctatcggtagaatatactctggaatcaatcaatgttccggtggtaaatatgtcttagatgccgatatagcaaaatgcttcgataa |
| | | gattaaccatgagtacctactgtccaaagtagattgtccatatctcatcaaaagaatcattaaacaatggttacaaagcggggtaa |
| | | tggacaatggtgtatttgaggaaaccgattcaggaacacctcaaggtggtgtaataagtcccttgttggctaacattgcactacat |
| | | ggaatgataaatgacacaatgaacaagtttcccaaatccttttacagaaatgggaaacaagtgagggataatcaacccaaaatc |
| | | atccgatacgcagatgacttcgtaattatacataacgaatacgaagtcatcctacaatgtaaagaaataattgcacaatggttaaa |
| | | aaaggctggactggaactaaaaccagaaaagacctccatcagacataccttaaaaagtattgaacacgatggacaaaccgttg |
| | | accctggtttcgattttcttggattcaacatcaggtcatattcaaagggaaagcaccgttcaggtaaaaccccacgtggcaagat |
| | | aataggaagcaagacactaatcaaaccaagcaagaaaaaaatactagctcaccacgaggctttaaaagaggtcattaaagac |
| | | aataaaaaagccccacaagcagcattaatcgcaagattaaatcccataatactaggatggtgtaactactatcgaacggtcgca |
| | | agtaaggaaacatttgcatccgaaaacaacgtactatggaatatgctaagagcttggacagttaacaggaaaaagaaaaaaac |
| | | accattgtatgaagcccttagaaaatatttctcatacggaaagtacggaaaatggacgtttcaaacagaagaagcagtcctctac |
| | | tatcatgccgaaacggaaataaagagacatcaattggttaaacctgacgcatcaccttatgacggaaactggacttactggagt |
| | | aaaagacggggaacatataccgggacaccagctagagtatcaagactcttaaagaaacaaaaaggtatatgccctcaatgta |
| | | agcaacacttcacacctgaagacttgatagaagttgaccacattatccctaaatcgaaaggtggtttggatacatacaacaatctt |
| | | caagctctacatcgtcactgtcatgaggccaaaagcaaaaatgactacctctatgattggcacttataatggctatgaatggaaa |
| | | gacgatgtattaacagtaccctagacaagggactaattgccgagagccgtgtgaggtgaaagtctcacgcacggttcggactg |
| | | ggagtcgtggtaggtgactaccacttcgacccctaatcaagcttatt |
| 3139 | Candidatus Kuenenia stuttgartiensis | cgttaattcggtacgccaagaagcgtatatatatcagttggtgcaaatccaacccgccaattgaccgttcgggcggtagtgaaa |
| | | gaggcagaaaacctcacgggagaacgtaaacagcgatgaatacgttcgagtcccctgtagcgagcagtcagaccgtagcg |
| | | caagcgaacacgcaggcttcgttacgctaaaaatacgatagatgagcctcccggattgggcgaaatctgtaatcgcataggta |
| | | gaaagagacaaacggtagaactatgcggtgtcgtcggagttgagagtgacggtatgactgtaaaaggtatatatatgaacttg |
| | | ggaggtctcattgttcaggttgatgaccgtaacgcatactataagggaagccgaagggtatagcgggataatgagaagtcgga |
| | | gatgcccatagtagtgaagatggtgaagacaacataacttcaccgtagcgaaggggcattacttcagttaacgtttacaaagaa |
| | | aggaggaaattgtattgggagacctacaagcaataccgaaatcgcaaatgagcgatgatgaaagagtccgagactttcagcg |
| | | taaactatatcgaaaagccaaacaggaagaaggatttcgcttttatgtattgtatgacaaggtgagaatgttgcactttttgagaga |
| | | ggcgtacaagcgctgcaaagccaatggaggaagcgccggggcagacgggattacatttgaagatgtggagtcatacgggg |
| | | tggagaagtttcttggagagataatagaggaacttgaaaacaaaacctacgaaccacagccggtactgagagtgtacatacct |
| | | aaaaccaacggtaagacacgaccattgggaatccctgtgataaaagacagggtagtgcagatgagtgtaaagcttgtaataga |
| | | gccgatattcgaagcagactttgaagatagctcctacggattccgaccgggacgttcagcgggcgatgctgtgaggaaaatca |
| | | aagaaaaactgcgagaagggaaaacagaggtatttgacgcagacctgagttcttattttgacacaataccgcacaaggaattgt |
| | | tactgttaatagggatgaggataagcgacaagaacgtactgcacttgataaaaatgtggttaaaagcaccggtgatagaggaa |
| | | ggcaaaccaggaggcgggaggaaaaataagataggaacaccgcagggaagcgtaatatcaccattattagcaaacatatat |
| | | ctgcacatgctcgacaaagcagtcaatagagagaatggggttttctataagtatgggataacaatcatacgttatgcggatgatt |
| | | gggtattgatggcaaagcgaatacctcgtgaagcattagactatctcaacaggttgttaaagaagttaaagctgagtcttaacga |
| | | ggataagagtaaaatagtaaaagctgaagaagagagctttgactttctcggacataccatatccttctcagaagatttgtttggta |
| | | ggaaacacaagaaatactggaacatagaaccaagcaggaaatcacagaagaaggttagggaaaagataggaaactaccta |
| | | aagagcaatgggcataaagcggcggaaaaagtagctaatgaactgaatgcaataacaagaggatggataaattacttcacaa |
| | | taaagggagtaacgtatccaaacaaggcgaaaagagacctacgatattatcttttcaggagattaacgagatattataagagga |
| | | aaagtcagcgcaggagcaagctctataatcgaggagcgtttaaggaattagtcaatagatattgactaatagacccgacaaaat |
| | | atgttcctgccagacaacctgtgaaagcttaagaagaagactgtcggaaagccgtgtgcgggaaaattgcatgcacggtttga |
| | | cgaggggggcagatgataagtatatggcggagatatggtggcactggcgggaaaccagccagcaaacagagaaaacaaa |
| | | cttctgcctaagttctagtcgtctgtttctactctaccgttcattag |
| 3140 | Candidatus Accumulibacter phosphatis | agcgccgggtgtgcgcccagcatgggcgcgatgtgatggggtggaagtcccctgtgggagtaccgacgtgacttcatgatg |
| | | aagccatgataaccactagccgaaggcaagggcaaggtcgtgaggacttgtctggaggaagccggagcgcaaaagtgcga |
| | | ggcgacggacagaaactgcatagaaggcctggtctgcgggcgagttggcaccccatgacgaagcccaccggatacggcg |
| | | gacaaggtaaatgcagcgcttgtgcacggaaacatcgcgttcttacctggggagacctgatcggcaagcgattgccgacgttt |
| | | cgcaaaagcagtggggatcgctgcttaacagaatgatccgggcgttgcgggaggctgccggccgcagacgagcgaacag |
| | | gcagcgagcgaagcgaaggccatagcgcgacggcgagcaatcgccagcgtgactgatcaggagtcagcagaggccata |
| | | gtagcgacaccgaaaggtaacgcgaagggccgaacatgaacagacaaggaggagccgtgagtcactcgaacccgacaat |
| | | gaacccgaccgggggagccggggatcggcgtgatcttgcccagccagccttgcacgacgccccgaaggaaggttgtatgg |
| | | ggaaggacttgatggaagccgtactctcaccggccaacctgaaacaggcgtggcgtcgagtgaaatccaatcgcggcgcac |
| | | cgggaatcgacggactgcgcattgaggacttcccggcgtatgcttgtgagcactggccggccatccgccagaccctgagcg |
| | | agggtcgctatcagccacaagcggtgcgacgggtgattatcccgaagcccaatggtggcgaacgcgcgctgggcatcccg |
| | | accgttgttgatcgggtcgtccagcaagccatcgctcaaatcatgacaccgatcttcgatccggaattctccgaatcgagctac |
| | | gggtttcggccccgacgctccgcccacggcgcactcaagcaggtcagagccgacctcaaggccggctatcgcatcgccgtt |
| | | gatctcgacttggcgaaatttttcgacaacgtcgatcacgacatcctgatggcccgagtggccagaaaggtgagcgacaagc |
| | | ggttgctggccctcatcggccgctacctgcgggcgggtgtcatgattggttccacgcttcaacccagcgagttgggaacgccg |
| | | caaggcggacccctctcgccgctgctggccaacatcctgcttgacgatctggatcggactctggaaggggaggccaccgc |
| | | tttgcgcggtacgccgacgacctcatggttctggtcaagagcgaacgggccggccagcgcgtcaaggccagtctcaccgcc |
| | | tacctcggccggcaactcaaactgccggtcaacgagaagaagagccaggtggcaaagatcgagcagtgcgtttttctgggct |
| | | tcaccttcaggaagaacaaactgcgctggtcggatgccgccttcgccgacttcaagcaccgcctccgggaactgacggggc |
| | | gaagctggggagtctcgatgccacatcggttcgagaaactcgggcagtacctgcgcggctggatgggctactttggcatctc |
| | | ggagtattaccgaccgattcccgaactggatgaatggctacgccgacgtgtgcgcatgtgctactggaaacagtggcgtttgt |
| | | gtcgcacgaagatcagccaccttctggcgctgggggtcgaccggcggacggcgatcttgacgggcgtcagcagcaagagc |
| | | tactggcatttgtctcggtcaaaggccacgcaagtcgggatgacgaacgattggttgagagcacaaggtctggtgagtatccg |
| | | gaatctttggatgaaggctcacggttacgcttaacaacgtcgtgtgcgccctgttgatgaaccgcccggtgcggacccgcatg |
| | | ccgggtggtgtggggagggccggttagacgccggcccttacccgattagggcctga |
| 3141 | Bordetella petrii | ctgcggcaaggtgcgccgtatcggcgcttagaagaagcgtagcgcaagctaccagggctactggaatgccctgtactcacc |
| | | ggacttatccctgcctgcaagggcttgaattccgaaaggaaggggggagtgcagcatgttcggaaaaggccttttacccaaca |
| | | gctattagcggggggggccagagtaagacgtgtatggtcaaagctgaattgcctgaagtctagttccaagcggtctaccagt |
| | | cccgacgctggcgaaagtagctgaaacgccagactcgcgagttgtgggaaatgttttgcctctcccacaaaccagtatcgcga |
| | | gcgcaaccccgatcaggggattacggaacgaacggacgacctaaccacgtcgcatctttgtctaagcgtaaatgcgggatcg |
| | | cctaaacaggtcgtaagacctgcatggcgacggagtcttcgtattacccgatgtggcctctgtaatggaggttgctggccgcag |
| | | cagtaagcggcaccgacatggctataagcgagcagcaatgcgcgtgaaaggcccactgtccggggggaaggaagacagc |
| | | ttatctgtaaccaactcatcgaaaaggaggttgtgctgatgctaaccgacactactaatcgaaggttggatgcgctgggaatctt |
| | | gtctcaacagggcaagcgcatcaacggccttagtcgtctcatggaaaacccaatcctgtggaagcaggcgtatgtcaacatct |
| | | atgcgaattccggtgcaacaacggccggcgtagatggctcgtcactggatggtatgtcctacgagcggctggccggcctgat |
| | | ggccgccgtcaaaagcggtaattatcgcttcaagcccgtgaggcgagtcctcattccaaagagcaatgggaaaacccgtccg |
| | | ctcggcatccccactggggatgacaagctggtacaggaggttgtacggatgcttctcgtgaagatctacgagccggtcttttcc |
| | | gacgactcgcacggttttcgcaacgggcgttcgtgccacaccgctctcatgcaagtccggcagaaatggacgggcatgaagt |
| | | ggatcgtgaacatggatatcaaaggttacttcgacaacatcgaccatgaagttctggtggatgtgcttgccaaacgcattgacg |
| | | ataagcgctttcttgggctgattcattcgatgctgaaggcgggctacatggaggactggaagttccatgacaccttcagcggga |
| | | caccgcaaggcggagttgtctccccggtcttggccaacatctacttgcacgaactcgacgagtacgttgcagggttgaaggcc |
| | | gaattcaaccgaggaaaccgccgggcgtcgaaccgcgagtacaagcggatcagtggtgcaattgaacggttgatgaaacgc |
| | | attgatgcctacaaagccgatggcgactcgccaaaggtcgaggaagcgaagcgtgaattggccgaactttatctacgccgca |
| | | aggcactgagcagctccgacccgatggacgccaattaccggcgtctggtgtatgtccggtatgccgatgattttctgatcggca |
| | | tcatcggcagtcgggacgaggcggtaaccgtcatgcagcgagtcgcaggtttcatcagcgacaaactgcacttggagatcgc |
| | | tgaggaaaagtccggtgtggttcacgcatcggacggagttcgctttctggggtacgacgttcgcacgtactccggtgatcgca |
| | | acgtgcgcaccgtccgttcaggacggagcatcacagcacgcagcgtatccgagcggatgcaattgcacgtaccggctgaaa |
| | | agctgcgtagtttctgccagcgaaagggctacggcgtctatgacgctcacacccccacgcacaggagcgaactgatctcact |
| | | gagcgaagcggaaatcgttcaaacctacaacgctgaaatgcgaggccttgccaactactacggtttggcgaacagcgtcagt |
| | | cgtgtcctgaacaagctccatcacctctggttgacgagtttgtggaagacattggcggcaaaacgtcgttcgaccgtcacgaag |
| | | gtagcgcggagcatgaagcgccagggtggatacgtactggctgttcggggtaactcggactcccacacttttcccatctacag |
| | | cctgaaagacataaggaaagaacccatcacgttccagagtattgacctaccccccaagacttgggttttcacgcatagccgctc |
| | | ggagttgatccagcggctcgaagcgaggaagtgcgagtactgcgggaccaccgaaggcagtttccaagtgcatcacattcg |
| | | gaagctgaaggacgtggacaaagggaagtacctctggccacaggtgatgtcatggcgccgtcgcaagactttggtgctgtgc |
| | | gttccttgccatcagaagctacacgcaggcgttctttgaaaggactgttcctagtcaagtaagtggagagccggatacgctgaa |
| | | aggtgtaagtccggttcggaggggggctgacgggtgatcctttacgggacgcccgcagcctaccctacacgaccgtcg |
| 3142 | Cupriavidus taiwanensis | agtacgattagtgcgccaggagactggcagagagtagtcgatgaaagttcgatacaggaaaggaatagcgaaccaccctgg |
| | | ccccgagtcatgcggcggcacccgcgagggtgcagccgaagcgttgacaggggagagtgcaggccagccattgagccg |
| | | cgaaatcattctgtctggagcgccgacgctgttgatcgaagcggaaggcaacatcgtcgagggcgctaaacgcgagttctcg |
| | | gcgggctccacgcggtcgaagaccctgagcatgcacagaagctctctgcacaggaactgggagatctcgccagtgcccac |
| | | cgagcagctggcggtgggcgggtcggggaaggccgatggccgtacgcccgacatcgacgctggcgagaagtcagatgct |
| | | tgcgtagtaccgaggaacgatccgaacaacgacgcggccgcaaagccggcgtctgcggaggatcgggagggaaggcga |
| | | gcagccaagaggaacgccgaacaatctcccgcgccccggacacagagccggtcccgcgcgtcgacggggttgaacggc |
| | | gtacgcgaagcagcctgtgcccaaaaggcatcgggcaagaaggtgcagttcaccgcgctgatgcaccacatcacgccgcg |
| | | attgctgatcgacagcttcatgcatttgaagaagtcggcggcggccggggttgatggagtgacatggcacgactacgaggaa |
| | | tgcttggtcgagcggataggcaagctctgggacgcggtgcaagctggccgctaccgtgccctgccatcaaggcgagtgtac |
| | | atacccaaagcggacggcaaacagcgtccgctaggcatcgctgcgctggaggacaagatcgtccagcaagcggtggtgac |
| | | agtgctcacaccaatctacgagtccgacttccttgggttttcctacgggttccggcccggacgtggccagcaccaagcgcttga |
| | | tgcgctgtgggtggggctgcattggaagaaagtgaactgggtgctcgacgcagatattcgctcgttcttcgacacggtcgacc |
| | | acgggtggatgatgcggttccttgagcaccgaattgcggataagcgcctgctgcggctcatccgcaagtggctgacggccgg |
| | | cgtcatcgagaacggggcgaagacggaaatacgggtaggtaccccacagggggcggtgatctcaccgctgctcgcgaaca |
| | | tctacctgcactacgtctttgacttatggctccagcgatggcgtcgccgcgacgctaagggtgacgtgatcgtcgtgcgatatg |
| | | ctgacgatagcgttgtgggcttcgaggccgaggctgatgcctcgcgattcctggaggccttaaaggcacgatttgctcagttcg |
| | | ggctgtcgctcaacgaacagaagacgcgagtgttgcagtttggtcgctacgcggccagcctacgcaagcgcgctggcttgg |
| | | gccggccgcagacgtttgactttcttgggttcacgcacatttgcgcgaccaagcggtcgaatggcggtttcatcgtcaggcggt |
| | | tgacatcgagcaagcggatgcgtgccacactcaaggcactgcggcaggcgctgtaccggcgccggcacgaaccgatcgc |
| | | agtggtcggcacatggctacgccgtgtgatgcagggctacttcaactatcacgccgtcccgggcaacaacctgcgattaagta |
| | | gattccgttctggggtctgccgagcctggttacatgccctccggcgacgtgggcaatatgggcgaatgtcctgggcgcgattc |
| | | ctccgacgggtcgcgccatatgtaccgtcagttcgcgttctacacccttacccaacacagcgcttcttcgcgtcatgactcaagg |
| | | caggagccgtatgcggtagccctgcacgtacggatctgtgcggggggggggggtaacctccgtccctaccgcgatcccc |
| | | gtcaca |
| 3143 | Clostridium tetani | gaatatactatagccatacaataaaagtgcgaaacgttatcctataagtaagaaagttttaaaattttcttacgaaaaggatagaac |
| | | ttaaaagttctaactgttctactaaagtaataagtgaaaatcttatttaaagcaaacaaccaagtagctttaagtctaagtcccctac |
| | | acaagttttatactactatgcaaaacttgtgaagctaggtaaggtcgtaatccgtgaaagtcggatgcggggctccttaaaagatt |
| | | actatggtaaacataagctaatccattaagatgcgatttatatgtattttatactgttaaatatttttgtgcttgtggcttggtataaaac |
| | | agttaagatgaagtacttaactggttttggaataattggttgttaaactaaaacattataaatgttagtggatacctaaggtaatca |
| | | aaaatagggataggtagaatggaacgtttgatgctgtatatgaagaggtttagtagaacctaggacacatatacgggctcagca |
| | | ggttcatagtagctatgatactcagccggaagtcaaattaattttgaaatacttctatggtaacataggagaaggataaaactgag |
| | | tgagccaaggaacctagtcggtaatagaaaagtggaagttaaaacaaatataagattttagaattaatttaattaatgaacggaat |
| | | taatttaatgatatttaaagttagacggttataaattaaacatttcaaaattaaaccatatccaaattcataaatatagctagatcatat |
| | | cactagtttaaaaataaataaatcatttcaaattactattaagtaaggtattaataccttacttaatagtaatctcattacataagagaa |
| | | ttactagattagcagacagattcataaaaactatatcaactaggacaatagaaaatatatttatacacttcctattatcgagcgaac |
| | | gccttatgcgatgaaagtcgcacgtagggtgtagaccaagcgaaatcctatgcatttaggatagtgaggtatagcaaaggaga |
| | | aaa |
| 3144 | Thermosynechococcus elongatus | aggttcttctgttgcgacgtggagctaaccagatgattgtctcgctaacccgaaaggttagaatgagaccagctgttcccccag |
| | | ctgtagcctagggaggcatgcgtgactggcaacggtcaggtatgaagccctcccgacaacgtagcctgaaccgtaaggttga |
| | | agccgaatccgtgaggaggaagcaacttcaccagtgtcaggtgatagggagctaggcttgagggtatggtgaacgtaagtga |
| | | agtgacgctagaagcctcgtgatcttagacaggccaaagacgctgataggcctgggccaaaatggcaaacggacaggactc |
| | | actgtttgttcttcagtgaggggggacgatagctccgtcggggcagagtcaccacctaacccctcatatgtcatctaaacggaa |
| | | cgtggtaagcccgtactttcgccttgataggctaagaacaaggcaagtggaccgcggggaacacccatggaagtgcgggta |
| | | aaggaggtgggaaaaagcgaatgccaatctgtaatggattggatagggattgaggatggtggcaggacatccaacatcatcc |
| | | cactcgaaagagggcagacttcccactggtccccagttacgagaaaactcacagaacctccctaatggtgacaaagcaaatg |
| | | gcggtgatcttgagtcactggtgcggtcaccaacctgctcaaaaggtagagaggctgtagatgagtatcgtaaaggttgcatg |
| | | cacaaccggttcctcttaaatgaggggtcctggggggctcgagccggatgcagggaaacttgcacgtccggttcctaggggg |
| | | ctagggggcagcaatgcccccctgctacccgaccgacatttct |
| 3145 | Azobacter vinelandii | tgaacgcttagtgcgccagttaaagctggcggaggatagtcgatgaaagttcgatactggtaagaaatggcgaatcaaccagc |
| | | ccccgagtcatgcacgttgcaccgtgaggtgcagggtgaagcgttgacaggggaaaccgatgggccagccattgagccgc |
| | | gtaatcagaaatccgggacgccgatgctgttaagcgaagcagaaggcaatacggggcatggcgttatacgccagtcatgcc |
| | | ctggtcctgcgcggtcggagaccctgcgcacgtcgggaagtccttcgcacagaaactgggagatctcagtgatgcccggtg |
| | | cgcatgcactgggcggagcaggcaaggccaaaagctgtaaccctgctgtctacgtcgctgagaagtcggatacgtccgtagt |
| | | acctgagaaaccgtcgaacaaaggggctagccctgcggagagggtggaggaaaggggcgtagccaaggggaacaccga |
| | | caagaaccccacgccccggacgctgagccggagcagttgcgtgtcgatgggacttgaaggtgtacgtgaagcagcccgta |
| | | gggacaagggtatgcagttcacggcattgttgcaccacatcacaccgcaactgttggcgcagagcttctacgcacttcgccgc |
| | | gacgcggcggtgggagtggacggtatgtcgtggcgagaatacgaagaagacctccaccagaggggggcaagttgcacg |
| | | cacggctccacagaggagcctatcgggcaacgccgtcacgggggtatatatccccaaagctgatggcaggcagcggcca |
| | | ctgggtattgcctctttggaggacaagatcgtacagcaggcagtcgtcaccgttctgaatgcgatctatgaggaggatttccag |
| | | gggttctcgtatgggtttcggccgggacgaagccagcacgatgcgctggatgcgttgacggtcgcgctcaagagccagaag |
| | | gtgaactggatactggacgcggacatcacgtcgttctttgacgagatcgaccatgaatggatgctgatgtttctggggcaccga |
| | | attgcagaccgacgcatgctcggactcatctgcaaatggcttcaggcgggagtaatggaggatggccgtaggttggctgcga |
| | | ccaaggggactccccaaggcgcagtgatatcgccgttgctggcgaatatctatcttcactacgtgctggatctgtgggcaagg |
| | | cagtggcgccaacggcatgcccgtggcgagatgattgttgtgcgctacgcggacgacagcgtggtgggtttcaggacgcaa |
| | | tggcaggcgcagcgtttcctggtgcagttgcaggaacgcatggccaggttcggtctatctcttaatgcctcgaaaacacggctg |
| | | atcgagttcggtcgctttgctgtacagaatcgcaggcggcaagggctgggcaaaccggagacgtttgatttcctgggcttcact |
| | | cattgctgtagtaccaacagaagcgggggatttcaaatcctgcggctgacggtcaagaagcggatgcgtgcgacactgcaag |
| | | ccatccggatagcactgaatcgccggcgacatgagccgatccgagtcgtaggtcaatggctcggcagtgtggttggaggata |
| | | tttcaactaccacgccgtgccgggaaacctgatccgtcttgacggttttcgggtggcggtttgccgtctttggcggcaagccctc |
| | | aaacggcgcagccagcgtaaccggctccagtggtcgcgctatggacgccttgcagacctctatataccaagacccagaactg |
| | | cacatccttaccctgaggagcgcttcgcgtcacgtacctgaggcaggagccgtatgcggtagttccgcacgtacggatctgtg |
| | | cggggggtggcaggtaactgccattcctaccgcgacctataggaac |
| 3146 | Bacillus cereus | gcaggtcgtggtgcgaatcgttcctaagtgaaaagcttaggcatcttagacaagggaaggccccattaagatagaactaatgat |
| | | tcgagagacagaatgatagagtaacgtcttgaaatgtttcccctaagtctccgacatgcattgaaaggtaggatgtttaaatgtgt |
| | | gaagctcggtgaagacggctgacagataccgtagtgaaaaatgtgttttaaccgaaagtccttttagtaagaggatgtatcccat |
| | | aggccgggggtccgtaaagtatctatggtgagaatgttatatgaaataactgacgaactttcgaattaacgagtctataaccata |
| | | caatcagaaatgatagtaaaccatattttgtgttgtgtgtgaggttaagtaaattcgcggctatgaacaaccttacagtattataggt |
| | | actgtctagcacgcaggctcatagaaggcacctaagggtaccatatagtggatagaatcattggaacgatgaaagctcaggac |
| | | gcagagaacctacattctgtgaagcggtggtaaggaaaaggaggaatcctataacttatctttatttgagtgatagcgatgtcata |
| | | gtagcgtggaatttatggaaacataaaggagcgaagggcattagtcaatattgtgaggaaaaagcaatattgatggcaagccg |
| | | tatgaagggaaactttcatgtacggtttagtgtgggggaaaaagcagagattatatcaaagttttacctatcacaataaggagag |
| | | aa |
| 3147 | unknown bacterium | gggtcgggccgaaagcgccgattaaaatcggtattggatagagaatgaaagagtcttatggtggaaggtttagcaaaccaca |
| | | ccagcctcgagtcatgcggcaattgcagtaatgcattggctgaagcgttgacagagggaagcacaggcgggctattgagctc |
| | | cgaaatcactcattctcgggtgccgaccacgtggtccgatggggaaggcaacacgttttgccgcgttattatgcgagctgtaag |
| | | acgacccggggagtcctagaaccttgcatgtgtggacatcctttacgcgagaatcgggagatccgtgagtcttcctcaaaatg |
| | | aaaggaacggccagtgaaagagaaattctgtacgcctggcatgaacgacttacggaagtcggacaacaacatagtacctga |
| | | gaagcttgcgaacaaggattttaaacatccgcggagcaggtggagggaagggcgttgaccaaggggaacatcataaacgct |
| | | gccgcagtctgtacacagggacaggtagctgcgtcgaccggcctttgtggtgtgtgtagaaaagctcagcaggacaagagc |
| | | gccagatttaacaatctttttcaccatattacgccagagctgttgacttcaagtttctacaacctgaatagaaaagcagctgctgga |
| | | gtagatgaaatgacatggcgcaagtacaaggaaggctcgcctggacgaatagcagatttaaatgaacgtgtgcataccggaa |
| | | gctatagagctaaacccgttagaagaagctacattaataaatctgacggccggaaaaggccacttggtgttactgctctggagg |
| | | ataagatagtccagcaggcagtctcaacgatattaaatcaaatctatgagaccgattttatgggtttcagctatggtttcagagaa |
| | | aaacgtagtcaacacaatgccctcgatgctttatatattggaatatcgaggcgtaagataaattacatacttgatgcagatatctct |
| | | ggattttttgataaaatcaatcatgactggttattgaaattcctggagcatcgagtagcggatcgcaaaatattgcggctgattaaa |
| | | aaatggctaaaagtcggtgtgattgaagatggtaaacgaacatcacttgaagttggtaccccacaagggtctgtgatttcaccc |
| | | gtgttggcaaatgtttacctgcactacgcacaagatctttgggcacaccaatggagaaaaaggcatgcagacggagacgtgat |
| | | tattgtgcgttacgccgatgatagtgttgtagggtttcaatacagaaaagatgcagacaggttcttgaaggatctgattgagagaa |
| | | tgggtcagttcggtctcgaactacacccagtcaaaactcgattaatagaatttggacgctttgcagtggttaatcgccggaaaag |
| | | aggcgaaagaaaacctgaaacgtttgatttcttgggcttcacacactcctgctccaaaacacgcactggacgattcttcatacgc |
| | | cgtaaaacgattaagaaacggtttgtccagaagtataaggaagtgaagcaagaactgaaggaacgtatgcatgatgatgtgaa |
| | | ggaaactggaaaatggctgaattcagtcatcaggggatttcaaaattactatgcagtccctggtaatatgaatttggtaaagagtt |
| | | tctatgaccaaacaacgcgggcatggctacataatctaagacgcagaagccacaaagggcaaagtttcagttggttgcgcttta |
| | | caaaattaatcaggtggttgataccgagagttcgaattgcacatccttttcctgaccagagatttcagcgttattacctgaggtagg |
| | | agcccagtgcggtagttccgcacgctgggatctgaacggggggtgctgggtaaccggcattcctaccgtgacgtatcaagtg |
| 3148 | Methanosarcinales archaeon | aaaatgataggagtgccgagataaatgcataatatccagttggtgaaaatccaacctgaggaaaggttagccgccagctcaga |
| | | actgaaccttgcgcccagcctggtaacaggctgtggtgaagccaggggaatgggatactgggccgcaacgcaagtgaagg |
| | | aattcagccacgaaatatttattgtcccggaggccgacgttgtcttgttgacggaaggcagtactctcatcgccgatagtcttatt |
| | | gggacatggcaaggcgatgcgagcccggcggggtctaagaccgtggcacggtatcaaacggatattatggaaactcggga |
| | | gacccagagtgttctccttacaggagtatgtcctatcaagcctataaacggtaagactgtacagacgacactctgggagtcgga |
| | | ccagttcatagtaccggtgatgtggggtaatgcccatagagggaagggactggcaggagcgcgatggaccggcagggaca |
| | | catcccccatacccagagatgggcaaggggtgtcaacaaaactgtggtccataactcaacgagccagagaagatccaacct |
| | | gtaagtttacgtctttagcgcacttacttacggcggattttctcaaggaatgtttccgggagttaaagaaagataaagctcccggc |
| | | gttgatggggtaacatggagaaagtatgaagagaacttagacgagaatacagaagatttggtaacaagattgatagcgaagca |
| | | ataccgaccacaaccagtaaaacgagcctatataccgaagtcgaacggggaacgaagaccgctaggaattccagctctcga |
| | | agataagatcgtccagttggcgataaagaaaattctggaggctatctttgaagaggacttctgtgacgtatcctacgggtttcga |
| | | cccaaccgcagttgccatgacgcactggacatggttgacatgatcatcatgaccaagccggttagctatgtggtggatatggac |
| | | attgcgaagttctttgatacggtggatcacgaatgcctgatggaatgcctgaaacagcgagtagtagacccaagtctgctgcga |
| | | ataatcgcccggtgcttaaaatcgggagtgatggaggaaggaaaatatctggagacggataaaggcactccgcagggaggg |
| | | atattaagtccgatacttgccaacatatacctccattatgcgttggacctttggtttgagaaagaggtgaaggagcagctaaagg |
| | | ggtttgctcagctcatccgttacgcagatgacttcattgtctgttttcaacacgatgatgaagccagagcgtttgggaaaacattaa |
| | | gggaaagactggctaaattcggactgacgatttcggaagagaagagcagaatcattaagtttggtcgctatgcctgtcagcaa |
| | | gcaagaaagcagagtaagaaatgtgcaacttttgacttccttggttttacgctctactgcgataaaacccgaaacggtaaatttaa |
| | | agtggggcgaaagacatctagcaagaagttcaggcagaagatgaaaataatgaatctgtggctaaagggcgttcgaaatcgt |
| | | gtgaaattagagttatggtggctattgttagcacaaaagatgattggacactaccagtactatggtataagtggcaatatacggg |
| | | gattacggagtttctactatcatgcgacggaactcgcctttaagtggataaacaggcgaagccagaggaagagctataattgga |
| | | gtcaattcaatcgttttctttcttttaatccactgccaaaaccgaagatatatcatttctatgccctatcaaaacaaaagtagaggatg |
| | | tactcctgaagagccggatgagggaaaacttcaagtccggttctgtgagggggctcatagtaaccttggagccaatactccaa |
| | | cagaaggtggactatgagccctactcgacatacagaccc |
| 3149 | Methanosarcina acetivorans | ggacggaatcgtgcgacgagaagttatgccatggattgcggtacaatccgcctttctatccattcagaatgtctctactctgatgt |
| | | gcatctctggcaacggatttgtgcaaagctcagaggacaacgaggtgtaaaactgaacctgacagttccaaaccgctaggga |
| | | aatggacatatggagaaccgaaaggtgaagattcacaggggtcgtgacttgcaacatgcgaaagcaggttgaattacgcatg |
| | | agccaaatggcatcgaaacccggttatgacaatccgattccgtgtcataacgaatggacgaatgggtttccggcctataggatc |
| | | ctcctaagtgttcagcaactatctatggtatagaactcggtaaaccatttgtggccctataaggtaggaattccgtgaggaaaacc |
| | | aataccacaggtggcagaggaaatggcaaaaagctaaagattccctgtaacggggaatatagaggagggaaaaaaaaactc |
| | | tttccttaatgcgaaagcatgcccacttgccattggtcacgaaagcaacagggattgttaattatgaatgtaagaaaaccagaga |
| | | taacttcggttacggaccttacagacaaagaactcacccaacaatggaaaagcattgattggaaaagagttaaagaagttgtta |
| | | ataaccttcagtctcgaattgcaagtgcagctaagaacggaaaatggataaccgtgaacaaactctcccgtcttctgacccggt |
| | | ccttatatgccaaactactttcagttcgtaaagtaaccactaacaagggaagccgaactcccggaattgatggaattatctggtca |
| | | tcgtcggcagataagatgcgttccgctttacaactaacgaacaagggctaccgtgcaaaaccattaacacgaaagtacattcga |
| | | aagaagaacggtaaactacgacctcttagcataccaactatgtatgacagagcaatgcaaaccctgcactctctggtgctaggt |
| | | ccaatcgaatctgctataggtgacaagacttcgtttgggtttaaaccttaccgctcaactaaagatgcttacgcctaccttcacatc |
| | | tgtttaagcaagaaaattgctcctgaatggattgtcgaaggtgatattaaagcctgctttgatgaaatcaaccacacttggatactt |
| | | gacaacatccctatggataaacgaatccttaaggagtttctaaaagccggatatgtcgagaattatcatctgtttcctacggaaaa |
| | | aggcacaccacaaggaagccccatatctccaataattggaaatatggccttaaacggcttagaaaacgccttagcaatgagatt |
| | | ttactccagatcagatggaacaattgacaaatctcatcaaaacaggcacaaagtcaattgtgcccgttttgctgatgactttgtgg |
| | | caactgctgattccccggaaacagctcttgaaataatcgatgtcatccaagaatttttagatcctcgtggacttaagctcagcgaa |
| | | gaaaagactcttgtaactaatattagtgaaggattcaattttttaggctggaacttcaggaaatacaaaggaaaactccttccgaa |
| | | gccatctaaagactcccaaagggaaattatcaagaaaatcagtgatgtaattcacaaagcaaaagcatgggatcaagaccgac |
| | | tgatacgaatcctcaacccaatcattaggggatggacacagtatcacaaccatacagtttcctctgagattttcagcaaacttgat |
| | | gatacagtctataacatgcttatctcttgggcgaaaagaagacactcaaataaaggtctcacctggataatgaccaaatactggc |
| | | ataaatccggtagcagaaaatatgtattctgcacagagctaaagacgttggagaggttctccaattccaaagttgttaggcaaag |
| | | actagcaagccttaacaaaaatccatttatcgacaaggaatatttcgaacaatggaaattcatggaataccaccgaaagaaacg |
| | | catcaccaacccaattctgttctaaactgacacccgaaagggtagtagtggctcgagccggatgatgggaaactatcaagtcc |
| | | ggttcctagaagacggggtagggagtaatcccaccctgttattcgacgtacgcgcca |
| 3150 | Methanosarcinales archaeon | tggtctctgtttgttgacaagaagttgcatcacagattgcccaactggctaccccttccattcggggtaatcctaccgcggaatgc |
| | | atggatggcaacgcccacgcagtaagcctgcggaacaacgtggaaaatcagaaaatccgattcggatcaactgctaggatac |
| | | gaatgctatgaagagtgtaatgcgaaccatcgaaagagtcgtaacacaggataagcgaaagcggactgttacgcttagacca |
| | | aaaggtacgaaatcagactataactgctgcagagacgttatagagggtggacaaacggtttccggcttacagatggcttctaag |
| | | gcgttcataattatctgtgacgtagaacttggtaagccccctaagctcccggaatatctgggtagagaccccgtgagggcgaac |
| | | gatggcatagggggtagaggaatcgataaaaagcgaatgacatcttgtaatgaggtgcatagaggttcaaaatttgccttaacc |
| | | caaaagggtgctgacttatcgagtggtgtctcattgcatgaacggaggcaaacctgtgaatgtaagtaattcaattacgccctta |
| | | aaaggcgagaagcttacagacaagcgaccaaaaccccggtgggacgataccgactggaagagggtcgaagaacatgttaa |
| | | caggctgcaaaccagaattgcaaaagcagttaagcaagacaagtggaatttggtcaaaaggttaagatttctgctaactaactc |
| | | gttttatgcgaaattgttggcagtnaagcgtgtaacccagaatagagggaagagaacagctggaatcgatggcgtgaagtgg |
| | | gcaacactgaactccaaaatgaacgctgctcttatattatccgatgtgaagtataaagcaaagccgttaagaagagtttacatctc |
| | | aaagcagggaacgacgaagaagaggcctttaggaatcccaaccatgtacgatagggcgatgcaagcgctgtacgcccttgc |
| | | gttactaccgattgcagagacgacatctgatccacgttcgttcggcttcaggatacatagaagcacacaagatgtgcgccaata |
| | | tgcatattgttgccttggtgggaaatactctgcaaaatgggttttggaaggggatatcaagggatgtttcgataatatcgaccatg |
| | | actggcttctgaacaacatcccgatggataaatcgattctaaagcaattccttaaggctggatttgtgtataatcgacatttgaatc |
| | | ctacccccgcaggaacacctcagggaggaattatctccccgatactggcgaacatgacactggatgggatggaaaaagctat |
| | | ctcatctgtgtattacgtcggtaagaatgggaaaatcgataaacatcgatataatctccacaaggtgaatttcgtgagatatgcgg |
| | | acgatttcatcgttaccgcaaactcagaagagacggcaaaggagattgcggagttgattaaagagttcctgaaggcacgaggt |
| | | ctggaattgtcagaggaaaagactcatatcacccatatcgattgtggctttgactttttgggctggaacttccgcaaatacggagg |
| | | gaagcttctgataaagccatccaagaactcgatggggaatctcatccgtaaaatcggtgatgtgatcaagcgagcgaaggcat |
| | | ggaaacaggaagacctcatcaacgtattgaaccccctcattactggctggtcgaattatcatcgatcggctgtagctaaggaga |
| | | tattcagcaaattagatcatattgtctgggatatgctctggaggtgggctaagaggagacacccggacaaacgtaatacgtggg |
| | | ttgctaatagatattggcattctgtgggaactcggaacagggtgttttctaccggaaggaataggttgaaactattctcggatacg |
| | | aagattgtccggtgtgctggcttgaaattggataagaacccctttattgaccaagactactttaacttacggaactgctgcccgat |
| | | actgaaagggttatgagatgcttgagcggtatgaggtgaaagtctcacgtaccgttctgagatgaggaggggcgggtaaccg |
| | | ccctattcttaatctgcggctatcctt |
| 3151 | Methanosarcina acetivorans | gctgatgactgtgcgccacgaaagcgaatccgtgtgacagaatgtcgcatagtgtaactatgctgcatagaagatgagggtgg |
| | | gcccctcggaatcgctatacaggtagagattccaaaccaccataagctgctgtggtcaaaagccatggtagtgagcgttatgg |
| | | aaaaggcgagacttgacctcgtcaggtatgtgctatggagacgaacgcaatgaaccactgataaagtgtcgaaagcgtaggg |
| | | atgtcatcaaaaccagggggtagtcgttaacctgggataagtctggaagaaacctgtttactgaccagatggtggccggcatg |
| | | aaggtggcgtgaacttagcacaggcttctgtgtggaacgtgggaacctgtacttcgatggtaagagaaaattccaagcggagg |
| | | acccgtaaggaggaaagtatcgaagcgatgtacaggggcggaacaatccgtagtagtgatgaagctcctgtaatgggagtg |
| | | aagcgaagggattgtgttatccagttttgaaaattggtcaacctgacaaatcaagggaggaacccatggacgaaacaaagcct |
| | | tatgaaatctctaaagatatagtacaagaagcttttcagagagtaaaagcaaacaaaggtgctgctggtgttgatgatgaaaaca |
| | | ttgcagcttttgaatcagatctaacaaacaatctctataagatttggaatagaatgtcctctggctgctatttccccccatcagtgaa |
| | | agcaatcgaaatccctaaaaagagcggcggaactcgcattctgggaattcccacagtactcgacagggtagcacagatggtt |
| | | acaaaaatctatttggaaccgcaattagaaccactctttcaccccgactcttacggctatagacccggcaagtcagctgcagac |
| | | gctcttgctgcaacacgtaagcgttgttggagatataactggttactggaattcgatatcaaaggattgtttgacaatattaatcac |
| | | gatctgctaatgaagcaagtcagtatgcatactgacaaaccatggatcattctctacattcagagatggctcaaagcaccctttca |
| | | gatggcagatggaacagtcaatgaacggacaaaaggaactccccagggaggcgttgttagtccgcttcttgcgaatctgtttct |
| | | tcattatgcttttgaccagtggatggatagtcatcatcggtataatccatttgaaagatacgccgatgatagtgtcatacactgccg |
| | | aagcaagaaagatgcagatcgactgaggattaagttagaaaagcgtttgtctgaatttggacttgaactacatccaactaaaaca |
| | | cggatcgtctactgtaaagatgacgatcggcaagaggattatcctgaaacaaaatttgattttctcggatacacctttagaccccg |
| | | taggtccaaaaacaagtatggaaaacactttatcaacttcactcctgcagtcagtaatactgctaaaaagtctatgcagcaagaa |
| | | atccataattggcgtatgcacctcaaacccgatttaacgttagaagacctatcccatatgtataatcctatagttagaggctgggtc |
| | | aactactatggtctcttttacaaatctgaactgtactgtgtactcaagcatatgaatcgtgccttgactcgctgggccttgcgcaaat |
| | | acaagaaactttcagggcacaagcgacgagcaagatactggcttgggaaaattgccagaagggatccaaaactttttgttcact |
| | | ggcaaatggggatttttcctgaggctggataatgggagccggatgagctgagaggttcaagtccggtgctgagagggcctgg |
| | | gggtgaaactcccccgggctactcaccttattgttac |
| 3152 | Bacillus cereus | gtgcgacacgtttctttataagtgtgcaaacacgaagtaggagggttatcaatttgatgacaacaacggattgactgcaagtagg |
| | | aatgaaagccgtcaggttgagctgaaactacttatctgatactcctatatgcaaggcgtgatagtagtcacaaattgtatgaagct |
| | | aggtgaagtcggctgaacaaaacctaagtgagaaatcatatggtaatggataggtcgggatgctacaaaacatctatggtgag |
| | | aatgtcctaacggactggcgaatgtacaggtttaaaggattaattcattagaaatgtgtatattgtcaacgagtagttatttcgacg |
| | | ctatctaccgaaaagtaagagtaaataatatgaaattcggaagatctaacgatgaggatgtaaagataacaggcttacagcaag |
| | | cacctaaagatatatgtatagctaagtcattcagaacgtggtaagcaagagactgtcacaaatgcctactaacagacaaggtgc |
| | | atataaggttctaacgaaccaaaattgctttattcttgtgaaggtggggacacagtaccgacgaagcatgtaacaaatgtggag |
| | | ggatagtccctagtcttgttcgttgaaaactaaatcaactggatataactcacaggatcgagtaagatgatgtgacttttcgtaaga |
| | | aaagggaatgaatacgttggtgagtacaacatggtttgtaatctagttgttttagtataaaaggataagatggggcgctgtatgcg |
| | | atgaaagtcgcacgtacagtgtcaagcgggggaaaagatggagataactttaaagtcttacctatcgcaacggataaaaag |
| 3153 | Bacillus thuringiensis | cctatcactagcgcgacacgttcctaagtgaaaagcttaggcactgtcgaactcaacagttcagcagtgaactgtcattctaaga |
| | | agtcaaatgaaggagtaacgtctggaagggcttcccttaatcctccgacatgcaggaaagtaggcaagtactgaactgtgtga |
| | | agctcggtgaagtcggttgaaggttaccgtaaattagtatctctaatacgaaagctatccagcggtggatggtgtaactgataga |
| | | ccggaggtctataaaacactcaaggttaggatgcgcgatgaactagaggcgatcgctagtaagcgcagacgaatccctgatg |
| | | gtacgggtctatatcgggagggaatcgaaaggttctctgacacaaataagtgtcgctactgtgggtgagtaaaactctcctttat |
| | | gaaagcccatatatcgttacaggcgttattaaggtagcaggctcataggggaaacctaaaagtgtatgtacagataagaatgac |
| | | ggaacgtggtaagctgccgacatggagggcttgttctctttgaagtgttgccaaggaaagtcacaatgagattagttgtcgatat |
| | | aacttggtttaacggcagtgaaagtggtggcacagtaccgatgaaacgtgtaatgaacgtggagggatagccactagtcgatt |
| | | gaagattgaaggttactattggttaacatggtttcgagtaagactaagagatgtaatgctccaaagtaataaggaggttacagcc |
| | | catgttaaagaaaaccaagctaagacataacgaatattatgatacacaaaaaaagtgtatgacaatttatactcgaacagtcttaa |
| | | cggtaacaatttctttcaattggaaacgatggaacgccgtatgcccggaaacgggcgcgtacggtgtggagtgggggaaaag |
| | | ctggagataatctcaaaggcttacctatcactatcgcgacacgt |
| 3154 | Onion yellows phytoplasma | tattgcaaaagggcgagaaagatgcctaatttcttagtaaattaaccacttacaactgcaattagtaatgcagtacaacatatttttg |
| | | gtctattttgtgaaagcaagatagattatagcagaaaataaaagcaaaaaaacgagaaactgtttatcgtttggatttgcgagttg |
| | | aaaatagtcatggaaaagctttgcaataagcctaagagaaaaaaacgggcagtagctataaggaaaaacagttgataccttata |
| | | ttttagggcagtttatatattttttgcgtatataaaatttactaacacttattgatttaagtggcaagcaaaattgctaaacctaaaacca |
| | | ccacaaccacataatttttttatttttgtatattaattgtggttatctctcctaaaactattaaggtaagaaattagagtaacttctatcttc |
| | | ctaaaagttatttgctaaaagaaagttatttgataaaagtataaaagcatctaaaataaacttaagtgtttaggctcaaggaaaaag |
| | | cagaatcgtagtagtcgtaagaaattcaaagcttagctggaatgcataagtggactgacttaccaaggaatttgggaaaaccaa |
| | | atacaaggcgaaagaagaaatatattttatataaacgctgtatgcttggaaacttgcttgtacggtgttgtgcgaggctcaaaaaa |
| | | acctaattattagctaataattaaggcgttttgtttctatcgcgataattactcaa |
| 3155 | Thermosynechococcus elongatus | agacattagggtgcgacgcgaaagctagccagatgattgtcccactagcccaacaagctagaacgggaccggttgtgcccc |
| | | caaccgtagcctagggaggcatgcgtgactggtaacggtcaggtatgaagccctcccgacaacgtagcccgaaccgcaag |
| | | gttgaagccgaatccgtgaggaggaagcaacttcaccagcgtcaggtgatagggagctaggcttgagggtatggtgaacgta |
| | | agtgaagtgacgccagaagcctcgttactctcagcaggccaaagacgctgacaggcctgggccaaaatggcaagtggacg |
| | | gctacacctctcctaattaggtgtacggggacattagctccaccggggtaaagtcaccacctaacccctcgcgtcatctggttg |
| | | gaacgcggtaagcccgtacttccgccttgataggccaagcacaaggcaagtggactgcgaggaacacccatggaagtgcg |
| | | ggtagaggagacgggaaaaagcgaatgctagcctgtaatgggttagatagggattgagaatgctggcaggacattcaacatc |
| | | atcccactcgaaagagggcagacttcccgtcggttccccattacgagaaaatctatagaacctccctaatggtgacaaagcaa |
| | | atggcggtgaccttgagtcactggtgcggtcaccaaccctcccaccaagggtaggcaggctgtagatgagtatcgtaaaggtt |
| | | gtacacacaaccggttcctcttaaatgaggggttttggggggcctgagccggatgcgaggaaacttgcacgtccggttcttag |
| | | ggggctagggggcagtaatgcccccctgctacccgactggccttgcg |
| 3156 | Thermosynechococcus elongatus | tgacaaagcagtgcgacgcgaaagctagccagatgattgtcccactagcccaacaagctagaacgggaccggttgttccccc |
| | | aaccgtagcctaaggaggcatgcgtgactggtaacggtcaggtatgaagccctcccgacaatgaagcccgaaccggaaggt |
| | | tggagccgaatccgtgaggaggaagcaacttcaccagcgtcaggtgatagggagctaggcttgagggtatggtgaacgcaa |
| | | gtgaagtgacgctagaagcctcgttactccaagcaggccaaagatgctgataggcctgagccaaaatggcaaagcggattg |
| | | gattcactgcttgcttttcagtgaacggggacagcaactccgccggggtatagtcaccacctaacccctcgtgtcatctggttgg |
| | | aacgcggtaagcccgtatcttcgccttgaacattcaaggcaggcaaaccgtaaggaatgctgatgggggtgcgggtagagg |
| | | aggtgggaaaaagcgaatgctagcctgtaatgggctagatagggattgagaatgctggcaggacattcaacatcatcccactc |
| | | gaaagagggcagacttcccgccggtccccccttacgagaaagtctatagaaccttcctaatggtgacaatgcaaatggcggtg |
| | | atcttgagtcactggtgcggtcaccaacctgctcaaaaggtagagaggctgtagatgagtatcgtaaaggttgcatgcacaacc |
| | | ggttcctcttaaatgaggggtcctggggggctcgagccggatgcggggaaacttgcacgtccggttcctagggggctaggg |
| | | ggcagcgatgcccccctgctacccgacaatggcggtg |
| 3157 | Thermosynechococcus elongatus | gaggcgtggagtgcgacacgaagccgcttaagctgactgtcccactagcccaacaagctagaatgggaccggttattccccc |
| | | agctgtagcctagggaggcatgcgtgactggcaacggtcaggtatgaagccctcccgacaacgtagcccgaaccgtaaggt |
| | | tggagctgaatccgtgaggaggaagcaacttcaccagcgtcaggtggtagggagctaggcttaagggtatggtgaacgcaa |
| | | gtgaagtgacgttagaagacccgttatcacatacaggccaaagacgctgacaggcctgagccaaaacggcaagcggacaa |
| | | gatacaacgcttccacgtcggtgtacggggatgttgatccgtcggggcatagtcaccacctaacccctcgtgtcaactaagcg |
| | | gaacgtggtaagcccgtatcttcgccttgaacattcaaggcaggcaaaccgcaaggaaagctgatgagggtgcgggtagag |
| | | gaggcgggaaaaagcgaatgctagcctgtaatgggctagatagggattgagaatgttggcaggacattcaacatcatccgac |
| | | tcgaaagagggcagacttcccgctggtccccgattacgggaaaacctacagaacctccctaatggtgacaaagcaaatggcg |
| | | gtgaccttgagtcactggtgcggtcaccaaccttacacacaaggtagagaggctgtagatgagtatcgtaaaggttgtacatac |
| | | aaccggttcctcctaaaagaggagtcttggggggctcgagccggatgcggggaaacttgcacgtccggttcctagggggcta |
| | | gggggcagtaatgcccccccgctacccgacaaaggctttc |
| 3158 | Nicotiana tabacum | cggcttttaagtgcggctagtctcttttacacatatggatgaagtgagggattcgtccatactctcggtaaagtttggaagaccac |
| | | gactgatcctgaaagggaatgaatggtaaaaatagcatgtcgtatcaacggaaagttctgagaatatttcattgttcctagatggg |
| | | tataaaaccgtgttagaattcttggaacggaacaaaataaagttgggtcgaatgaataaatggatagggctgcggcttcaattaa |
| | | attatagggaaagaaagaaaaagcaacgagcttttgttcttaatttgaatgattcccgatctaattagacgttaaaaatttattagtg |
| | | cctgatgcgggaagggtttcttgtcccatgagtggattctccatttttttaatgaatcctaactattaccattttctattacggagatgt |
| | | gtgtgtagaagaaacagtatattgataaagaaagttttttccgaagtcaaaagagcgattaggttgaaaaaataaaggatttctaa |
| | | ccatcttattatcctataacactataacatagaccaattaaacgaaacgaaaaaaaaaagagatgatagagaatccgttgagaag |
| | | tttacctgtatccaaggtatctattcttactaaaatactttgttttaactgtatcgcactatgtatcatttgataaccctcaaaatcttccg |
| | | tctttggttcaaatcgaatttcaaatggaagaaatccaaagatatttacagccagatagatcgcaacaacacaacttcctatatcca |
| | | cttatctttcaggagtatatttatgcacttgctcatgatcatggtttaaatagaaataggtcgattttgttggaaaatccaggttataac |
| | | aataaattaagtttcctaattgtgaaacgtttaattactcgaatgtatcaacagaatcattttcttatttctactaatgattctaacaaaa |
| | | attcatttttggggtgcaacaagagtttgtattctcaaatgatatcagagggatttgcgtttattgtggaaattccgttttctctacgatt |
| | | aatatcttctttatcttctttcgaaggcaaaaagatttttaaatcttataatttacgatcaattcattcaacatttccttttttagaggacaa |
| | | tttttcacatctaaattatgtattagatatactaataccctaccctgttcatctggaaatcttggttcaaactcttcgctattgggtaaaa |
| | | gatgcctcttctttacatttattacgattctttctccatgaattttggaatttgaatagtcttattacttcaaagaagcccggttactccttt |
| | | tcaaaaaaaaatcaaagattcttcttcttcttatataattcttatgtatatgaatgcgaatccactttcgtctttctacggaaccaatctt |
| | | ctcatttacgatcaacatcttttggagcccttcttgaacgaatatatttctatggaaaaatagaacgtcttgtagaagtctttgctaag |
| | | gattttcaggttaccctatggttattcaaggatcctttcatgcattatgttaggtatcaaggaaaatccattctggcttcaaaaggga |
| | | cgtttcttttgatgaataaatggaaattttaccttgtcaatttttggcaatgtcattgttctctgtgctttcacacaggaaggatccatat |
| | | aaaccaattatccaatcattcccgtgactttatgggctatctttcaagtgtgcgactaaatccttcaatggtacgtagtcaaatgtta |
| | | gaaaattcatttctaatcaataatgcaattaagaagttcgatacccttgttccaattattcctttgattggatcattagctaaagcaaac |
| | | ttttgtaccgtattagggcatcccattagtaaaccggtttggtccgatttatcagattctgatattattgaccgatttgggcgtatatgc |
| | | agaaatctttttcattattatagcggatcttccaaaaaaaagactttatatcgaataaagtatatacttcgactttcttgtgctagaactt |
| | | tagctcggaaacacaaaagtactgtacgcacttttttgaaaagatcgggctcggaattattggaagaattcttaacgtcggaaga |
| | | acaagttctttctttgaccttcccacgagcttcttctagtttgtggggagtatatagaagtcggatttggtatttggatattttttgtatc |
| | | aatgatctggcgaattatcaatgattcattcttagattttctaaatggaaatttgtttctaaatgatgaagagataaaaaaatttcacta |
| | | ttctgaaatgttgattgtaatagtaattaaggggtaaatcaactgagtattcaactttttaaagtctttctaatttctataagaaaggaa |
| | | ctgatgtatacatagggaaagccgtgtgcaatgaaaaatgcaagcacggcttggggaggggtctttacttgtttatttaatttaag |
| | | attaacatttattttatttaacaaggaacttatctactccatccgactagtt |
| 3159 | Euglena myxocylindracea | cttttaggtgttgtgttttaatcattatataagaatttacaaaaaaatttgtgactttacaaatcatattaaaacaaagttttaactgagttt |
| | | tttattttatcttttaataaactaatgttttggaataaatcgtttgtggtggttagtaatttgcaaaaaaaaattttcaagagtagttattcc |
| | | aatgattttaagcgttccaaaagctttcaggctttaattttaaaatcaaatgctgcaaaatacttagcaattcgctatgttctacaatct |
| | | aatttggataaaaaaaatttaactattgatggcaaaactactttaagttatttagagcgtatctcgttattagagtttttaaaaattaatt |
| | | ataataatcggtttcctcaaactatacaaaatgtgggttctttgaacaaaaatggtgattttaagttaacatttacttgtacgatatcag |
| | | atcgtgtatggcaaacttttttaaatttttctatgcaaccggctcatgattctacttttcatccctttaattttggctttcggtttactacgg |
| | | aaaacttttttatcctaaaagtcttacttttaaatttaaaaaatcaatctcttcctttacaaaaacgtatatactatttttctttagaaaata |
| | | gattggttgattttgattttctttatttagtgaaaaagttaagaatttttagaactattaaaattggaatctttcgtttattaaaaaaaaact |
| | | actctttgcagtattgtgaagataagtatgatacattttctacattgttatttaatgttcttttggatggcttgctagattttggtaatatttt |
| | | tcattatggttatcattttttgttaattctcaaaccaatagagagcgagtttgttttttttaataatatttcgtgttttttttctagtataggtgt |
| | | ttctttttcagggaagtattcattactttctattacaggtttcgattttttatactggagctttacaaaaaaaggaaacttttatatttcagt |
| | | tccttccacaaaaaactactttaattttcttggacgagttaaatatatagttaacaactcgaattatggtgtaaaaatcaaagctgcta |
| | | aattattcccgataatcaaaaattggtatgtttatcataaatattcttttaagaaaggttgtagctctattattttttctataaaaaagaaa |
| | | gtattaaaagcttttgttaaagagtctaaacaagatatttattctgctaagagattattggataaatgctttttttttgttgttattcacagt |
| | | cttaaaattatcaagcaaaaaaaattttttcttgcaaaacacttagtttttcgcacaggttatttaaaatttcaatgtattcactgtggcg |
| | | tgaatttaattttctcataatgtttctaaaattaataatgttttcccttaatttttctagtcaagattttatctatggcctaaaatataatttttt |
| | | cttaacaaaatctttcctttaattaattttgaaaatctcaacggattaatcttttctcatgtagc |
| 3160 | Methanospirllum hungatei | agttaatatagtgcgacaagaagcagtaaatcggattgcgaacccggtttaaaatcggtaacgccacccaatccattctgggcg |
| | | tccctacctgcacgtgcgtgtttagcaataaacgggtgcgaagctgcaggaacaacgtggtgattcccatagtctctttgggata |
| | | agctgccagggaaaggtatcatggacaaacaaacgttgaaccgctgcaggatcgttaattatatcaggaggaaagagatttgc |
| | | ccctgtccccactgagggagaatcacggtatcagtagcttgaattatgctggtacgaatggacgaagctgattccggtctatgg |
| | | gcgggtcctaaaataccacaattatccgatttattgaacttggaaaacccaatacgctcctaaaaaaaaaaaaaaaaaattttttttt |
| | | aggtaagaaccttgtgagaggaaccacatgcgtagtgggagtaggaggctgcaaaacgcgaatgcccggctgtaatggctg |
| | | tggataggggttcaaactttgccctgacctgaaagggtgccgacgtgcagctggtttttcacggcgaacaggatggtttcaatg |
| | | aacgggataagttcaattacgcataaaagcgaggacatctcggacagaaaacttgcaaaacaatggaagaaatttccatttgct |
| | | aaagcaagagattatgtaaagcgacttcagacacgtatcgcaaaagcagtgaagaacggccaatatcgacttgcaagacgac |
| | | tccagtatctgcttacacattcgttttatgcaaaaatgttggcagtacaacgagtaaccaaaaacagaggtaaaagaagtgcagg |
| | | agtagatggggaaaaatggaccacccctgaacagaaaatgaaagcagcattaacgctttcggacaagggctatcgggcaaa |
| | | acctctccggagaatctacatccctaaaccgcaatcgagtaaaatgcgacctctttcgattccaaccatgtatgaccgtgctatg |
| | | caagctttgtatgcaatggctcttatgccttgggctgagaccacagcagacaagacatcattcggattccgaatgaagcgaaat |
| | | gcacaggatgctgcttcatacacttttcagtgcttaagcagaaagacttcaggtcaatggatattagaaggtgatatccgcgggt |
| | | gcttcgataatttcgcacaccaatggatgcttgataacatccctcttgaccaaagaatccttaaccaattcctgaaagccggttata |
| | | tttatgatggaatactctaccgtaacaagtcaggtacgccccaaggcggcttaatttcccccttattggctaacatggctcttgac |
| | | ggcatggaaagaatgttgaaagaacactttcccggaaataaggttcatctcatacggtttgcagatgattttctcgtaacggcag |
| | | actcacaggaaacggcactccagtgcaaggaactcatcactgaatttcttcatgaacgagggcttgaactctctgaggaaaag |
| | | accaaaatcgttcatatcaacgaggggttcgatttcctgggctggaatttcagaaaattcaaaggcaaattcctgatacaaccttc |
| | | gaagaaagctattgccgcaatcattgataaagtaagggtaatcattaagtcggcgaaggcctggaaacaggaagaccttatca |
| | | aagccctaaatcccgtaattaagggatgggcaatgtatcaccggacggtttctgcaagtatgacctttgggaaacttgactgggt |
| | | tgtccgaaatatgttgtggagatgggcaaagcgccgtcataataataaagggaagagatggattgccagaaaatactggcacc |
| | | caacacttaccagaaaacaggtctttagaacctctactcttactctcgaaaacttctccaacaccaaaattcaataccgaaaattc |
| | | ataaaactggatgcaaatccgtttattgataccgagtattttgaaaacaggccaggagttttcctctcaaagcaaagatcgatacg |
| | | gatgttccttcactacgcccataaaagcgggtagtgaaaaacttgagcggattgcggtgaaaatcgcacggtccgttcttggaa |
| | | ggctgggttagggtaacctaacctggctattcgacatacaccaaa |
| 3161 | Methanosarcinales archaeon | gcggatgactgtgtgccacgaagctgcacgagagatgagggtttggcccctcgaagcctgcttgcaggagcaggcttcaaa |
| | | ccgccttaagctgctgtggttaaaagccatggtggtgagcgttaaggaaaaggtcccacaagatgggatcaggtgtggatcat |
| | | ggaaacgaacgtaagtgaaccgctgaggaagtgtcgaaagcgtatggacggcgtcaaaaccaggggctggtcgttgacctg |
| | | ggataagtctgggggaaaacctgcttactgcccagatggcgtccggcataaaggcggcgtgaacatggtcctggctctcgtgt |
| | | ggaacgtgggaacctatcccctaatgttaagggagaaacacaagtgggaggaccccacaagtgtgagagtaccgatgcagg |
| | | ggaataggggggaccagcccgtattagtgatgaagtctctgtaatggagatagagcgaaggggttgggtcatcctgcttgat |
| | | ttaaagatcaaccaggagatgggaggaatctttgatgaaagcaaagccatttgagatttccaagaaagttgtattggaagcgtg |
| | | gaaagaggtgaaagcaaaccgtggagcagcaggtgtagataagaagtcgattgcggattttgagaaggatcataagaataat |
| | | ctttacaagatctggaatcggatgtcatcggggagctactttcctccaccagtgaggacggtagggataccaaagaagagtgg |
| | | gggagagaggttgttaggcatacccactgtggctgacagagtggcccagacagtggcaaagaggtaccttgagcctttggta |
| | | gagccatactttcacaaggactcatacgggtacagacctggtaaatcagcgattcaagcagttggagtgacgcgtaagcgttg |
| | | ctggaggtatgattgggtgcttgaattcgacatcaaggggttatttgacaatattaatcatgacctcttgataagggcggtaagga |
| | | agcacacaaactgtaaatggatgcttttgtacatagacagatggttgaaggctccatttcagggagaggatggcacgctagtgc |
| | | agagagaaaaaggaacaccccaagggggtgtgataagccccttgcttgccaatctcgttttgcattatgtgtttgataaatggat |
| | | ggagcgaaattattcgcaagttccattttgccgctatgctgatgacggagttgtgcactgtcggtctgaagcagaggctttaagg |
| | | ctaagggaaaatctggaggctcggtttgggaagtacaatctcgagcttcaccctgagaagacgaggatcgtctattgtaaagat |
| | | gacactcgtcgagatgagtatcccaacacgagttttgattttcttggctttacattcaggccaaggaggtcaaagaatcggtggg |
| | | gtaagtatttcatcaacttcactcctgccgtgagtaataaagcggcaaaggtgatgaggcagaaggctcgaagatggaaaata |
| | | cacctgcgaagtgacatgtccttagacgaactgtcaaggatgctcggtcccattatcaggggctggatcagttattacgggagc |
| | | ttttataaatccgcgctgtacccaattttgcgacatctgaacaggatattagttagatgggcaatgaggaaatttaagaggtttaga |
| | | cgccatcgtaggcgtgcggaatactggttgggggagattgcccaaaggcagccctggctatttccacactggcagatgggtg |
| | | tgagacctatggctggatgatgggagcccggtgagctgagaggttcacgccgggttctgagaggacgtaggggggaagttc |
| | | ccctgcgttactcacctcattgtctg |
| 3162 | Methanosarcinales archaeon | ataaatgtgggagtgccgagctaaatgcataatatccagttggtgaaaatccaacctgaggaaaggttagcagccacctcaga |
| | | actgaaccttgcatccagtctagtaacaggctgtggtgaagccagggggatgggatactgggccgcaacgcaagtgaaggg |
| | | attcagccacgaaatatttattgttccggaggccgacgttgtcctgttgacggaaggcaatactctcgtcaccgctagtcttaata |
| | | ggacatggcgaggtgatgcgagcccggcggggtccgagaccgtggcacggtatcaaatggatattatgcgaactcgggag |
| | | gcccagaatgttctcctttcaagagtatgtcctatcaaacctataaacggtaagatggtacagacaacactctgggagtcggacc |
| | | agttcatagtaccggtgaagcagggtaatgccttcagagggaagggactggcaggagcgcgttggaccggcagggacaca |
| | | ccttccatactcagagatgggcgagaggtgtcaacaaaactgtggtccataactctacgagctagagaagatccaacctgtaa |
| | | gtttacgtctttagcgcacttacttacggcggattttctcaaggaatgtttccgggagttaaagaaagataaagctcctggcattga |
| | | tggggtaacatggagaaagtatgaagagaacttagacgagaatatagacaatttggtaacaagattgatagcgaagcaataca |
| | | ggccacaaccagtcaaacgagcctatataccaaagtcaaatggggaacgaagaccgctaggaattccatctctcgaagataa |
| | | gatcgtccagttggcgataaagagaattctggaggctatctttgaagaggtcttctgtgacgtatcatatgggtttcgacccaacc |
| | | gcagttgccatgacgcgctggacatggttgacatgatcatcatgaccaagccggttaactctgtggtggatatggacattacga |
| | | agttctttgacatggtagatcacgaatgcatgatggaatgcctgaaacaacgtgtggtagacccaagtttgctgcgaataatcgc |
| | | ccggttcttaaagtcgggagtgatggaggaaggaaaatatcagaagactgatagaggcactccacagggagggatattgagt |
| | | ccgatacttgccaacatatacctccattatgcgttggacctttggtttgagaaagaggtgaagaagcagttaaatgggtttgctca |
| | | gctcacccgttacgcagatgacttcattgtctgttttcaatacgatgatgaagccagagcgtttgggaaggcattaagggaaaga |
| | | ctggctaaattcagactgacgatttcggaagagaagagcagaatcattgcgtttggtcgttatgcctgtcagcaagcaaggaag |
| | | cagggtaagaaatgtgcaacttttgacttcctcggttttacgctctactgcgataaaacccgtaacggtaaatttaaagtggggcg |
| | | aaagacatctagcaagaagttcaggcagaagatgaaaataatgaatctgtggctaaagggcgttcgaaatcgcatgaaattag |
| | | agttatggtggccattgttagcacaaaagatgattggacattatcagtactacggtataagtggcaatatacggggattacagag |
| | | tttctactatcatacgacggaattcgcctttaagtggataaaccggcgaagtcagaggaagagctataattggagtcaattcaatc |
| | | gttttctctcttttaatccattgccaaaaccgaagatatatcatttctatgccctatccaaacaaaagtagaggatgtactcctgaag |
| | | agccggatgagggaaaacttcatgtccggttctgtgagggggctcatagtaaccttggagccattactccaacagaaggtgga |
| | | ctatgagccctactcgacgcatctgtgt |
| 3163 | Methanosarcinales archaeon | gttctctgcctgttgacaagaagttgcatcacagattgcctaaccggctgccccctccattcggggtaatcctaccgcggaatgc |
| | | gtggatggtaacgcccacgcagtaagcctgcggaacaacgtgaaaaatcagaaagtccgattcggatcaatcgccaggata |
| | | aagaacgctatgaagagtgtaatgcgaaccatcgaaagagtcgtaacgcaggataagcgaaagcggactgttacgcttagac |
| | | caaaaggtacgaaatcagactataacggctgcagagacgttatagggaatggacagacggtttccggcttacaggtggcttct |
| | | aaggcgttcataattatctgtgatgtagaacttggtaagcccaatatgctcccggaaaatccgggtagggatcccgtgagggcg |
| | | aacgatggcatagtgggtagaggaatcgataaaaagcaaatgtcgatctgtaatggatcgaatagaggttcaaattttgccttaa |
| | | cccgaaagggtgctgacttatcgagtggtgtctcattgcatgaacggaggcaaacctgtgaatgtaagtaattcaattacgccct |
| | | taaaaggcgagagacttacagacaagcggccaaaacccctgtggaatgataccgactggaagagggtcgaagaacatgtta |
| | | acaggctgcaaaccagaattgcaaaagcagttaagcaaggaaagtggaatttggtcaaaaggttaagacatctgctaactaac |
| | | tcgttttatgcgaaattgttggcagtaaagcgtgtaacccagaatagagggaagagaacagcgggaatcgatggcgtgaagtg |
| | | gacaacacagaactccaaaatgaacgctgctcttaaattatccgatacgaagtataaagcaaagccattaagaagagtctatat |
| | | cccaaagcctggaacgacgaagaagcgacctttaggaatcccaaccatgtacgatagggcgatgcaagcgctgcacgctct |
| | | cgcgttacaaccgattgcagaaacaacagcagatccacgctcgtttagcttcagggtgcatagaagcacacaagatgcgcgt |
| | | caatacgcattctgttgccttggtgggaaatacactgcaaaatgggtttgggaaggggacatcaagggctgctttgataatatca |
| | | accatgactggcttctggacaatattccgatggataaattgattctaaagcaatttcttaaggcgggatttgtgtataatcgacattt |
| | | gaaccccaccacagcgggaacaccacaaggaggaattatctccccgatactggcgaatatgacactggatgggatggaaaa |
| | | agccatcgcatctgtgtaccaagttggtaagaatggcaaaatcgataagtgtcgatataatccccataaggtgaatttcgtgaga |
| | | tatgcggacgattttatcgtcaccgcagactcgaaagagacggcaaaggagattgctgagttgattaaagagttcctgaaggc |
| | | acgaggtctggaattgtcagaggaaaagactcatatcacctatatcgattgtggctttgactttttgggctggaacttccgcaaat |
| | | acggagaagagcttctgataaagccatccaagaagtcgattgggaatatcatctgtaaaatcggtgatgtgatcaagcgagcg |
| | | aaggcatggaaacaggaagacctcatcaacgtattgaaccccatcattactggctggtcgaattatcatcgatcggctgtagct |
| | | aaagagatattcagcaaattagatcatattgtctggaatatgctctggatgtgggctaagaggagacacccggacaaacgtaat |
| | | acgtgggttgctaacagatattggcattctgtgggaactcggaactgggtgttctctaccggaaggaataggttgagactattct |
| | | cggatacgaagattgtccggtgtgccggcttgaaattggataagaacccctttattgaccaagactactttaactttcggaaccgt |
| | | tgcccgatactgaaagggttatgagatgcttgagcggtatgaggtgaaagtctcacgtaccgttatgagatgagaaggggcgg |
| | | gtaaccgccctattcttaatctgcgtgctctgcg |
| 3164 | Amoebidium parasiticum | taacagacgcatgaatgtcttggaatgaaaatggaaacccacgtattggattcaaacagggtgaagtaaactttcctttttatggc |
| | | atgtgtatttagaacttatatctttaacatctagtttccctggcattcgacaatcaatagtaagaggaaagtctcataattcacactctt |
| | | ttgatacaaaaaatctccaatgcttaaatgaactaagcaatattctgtacactaatataaatggtaaaatggtaaaatctattaaagg |
| | | agaactatttgattatttaaactacagaacattagcgtacatgataatgggagatggagcgcgcaaaaataaagggtttgtaatat |
| | | gtctcgagaatttcacacttcaggaagtcgttttactattaaatatgttagcaatcaaatttaatttggaggacctaactattcaaact |
| | | gaagactctaaaccgtctccttttaaaaaagaaaaagaggaaattaagaagatttatagaatctactttggaatagagggaacca |
| | | aaacattattacctttcattaaaccttatttactagaccattttgattatcgcatatcgggaatgactaaagcacagtatgaacaacgt |
| | | ggtgtaaagcataaatcttaggtagtaggag |
| 3165 | Mesostigma viride | agaaaattggatgccgcgatttttttgcataagtgatccacatttttccctaaattcttctttatttctttccgtggtcactgcaatttctg |
| | | cattatcagatcctttagatatttggatagaaaaatcgttcggaaatgaaaaatttttttattcacgttttatggatcaatgtattgtagc |
| | | atgtttttcagacgctgattttttaaattcaaaatttgaaaaaatatcacaaaaatcttgttggaaattttctaaatgtttttccactgttcc |
| | | ttttttgggtggcacgctaattcactcttcaacaaggaaacccattgaatctaatgagctaaaaagtattaataatattaacagttctt |
| | | ggaaatggaaaatttcatggaatgttcttataaggttttgtcaaaatgaaagatattttcatcaaaattctgcaaaacctacggctgt |
| | | gccctaccttgttccacgaactcatgaagagattttggctcattataaaaaattaatttttttttttatctttattacaatcctatgcaag |
| | | atcaccaagcattcaattgtttagtagatggtttaaaggaatcatgtatattgacattggcccttaaatataaaatgaaaagtagaac |
| | | taaaataattcaaaaactttttcctcaaaagctgtaggagtttttct |
| 3166 | Azotobacter vinelandii | gcatgatgtagtgcgacgagagtcgctataggtaggagtacctagcgggctacatcctaggcggtcaccacatggttggtgg |
| | | ccgcttttcccgcaaaacagctctaaaaacccgagatccgggcgacaaaagcccggattcagggtgccgtcaaggcgctaa |
| | | ggattcggcaattcgttgcctaaatatccggtcttccaccgtggagaccataaaacgatattcgtactgagcctgtatttcgcgttt |
| | | tgagtacaggggaggtatgggagatcgtgtccaaaagctggctgctcctatccgtagggcgcaagccatagctgtcattgtct |
| | | cacccgaaaaccgcaaggtggccggggcgcgagaccttctgttcagccagaaagtagcctagggacagtgcgtcgccggt |
| | | aacggcggggtgcgaagcgtcctgacaatgtagccccatgatgtctgtggtgcgtggtcgccgcgaggcgccatgcagacc |
| | | ctgcgagcaacacgcgggcgaggcgctaggctgggtggtacggccaacgtaagtgaactgctgataaacaccgtcaaccg |
| | | catggagccaaagttgctgacaggcttcgaccaaaagggtgcgtgactggttgtccctcttacatctggggacacgctgacat |
| | | cgagtcaccggtgaacaggcagggcctaacccactcttgtgatggttatggaactcggtaagcccgtattttcgccactggctg |
| | | gtagagcgggggcaggtcgaccgcaaggaaggctgttgaaagtgcgggtaaaggaggtcggaaaaagcgaatgccgccc |
| | | tgtaacggggctggataggggccgttgccccgacccgaaagggtgcccacttccgactggtctttgatcgcgaaaaggcttgt |
| | | aaagccataaaagctggcgtcgttcggtccgaccggcggcgtctgaacagaaatcctcgcggggtagtgaactcactccccg |
| | | caagggcgacgtgcgtccgctgctcctccggggaaatcttctttggaggaaagcaagatgagtgcgtctgaaaaggaagtac |
| | | ctgcgtcctccctcccggccagcagttggcacacgatcgactgggcaaccagtcatcggcgggtgagagggctacaggtac |
| | | ggatcgcgaaggcagccaagaatcggcaatggcggcaggtgaaaaccttgcagcgcatgctggtccgctcgttcgccgcc |
| | | aaggcattggcggtcaaacgggtcactgaaaaccggggtcgcaggactccgggcgtcgacggggaaacctggagcacgc |
| | | ctgaaagcaaatggaaggctatttttcggttacagcgcacgggctaccggcccaggccgttgcggcgggtgtacatccccaa |
| | | agccaacggccagcgccgaccgctgggaataccgacgatgctggatcgggccatgcaggcattatacctgctggcgctcga |
| | | accggtgtccgagaccacggcggaccggaattcctatggctttcgtcctcaccgttccacggcggatgcaatcgagcaattgtt |
| | | cgtcaacctcggtcgcaagcactccgcgcagtgggtcatggaaggggatatcaaaggctgtttcgacaacatcagtcacgatt |
| | | ggctgatcgccaacgtcccgctggacaaggcggtcttacggaaatggctgaaggcgggatacctggaatccggccaattga |
| | | accccaccggggcgggaactccgcaaggaggcatcatctcgccggtgctggccaacctggcgcttgatggtctggaaaag |
| | | gcgctcgaatcgcgattcgggcaacgcaacaccaaagccagctacaagaccaaggtcaactatgtgcggtatgcggacgat |
| | | ttcgtcatcaccggcatctcgaaagagctgctggtgaatgaggtgaagcccgtcgtcgccgccttcatggcggagcgcgggtt |
| | | gagcctcgcggcggaaaagtcgctgttcacgcatgtatcggaggggttcgatttcctcggccagaatgttcgcaagtacggtg |
| | | acaagctgctgatcaagccagctcaccggaacgtcaaggcatttctggccaaggtcaaggcgttgatcgaggggaacaaga |
| | | cggctccggccagtctgttgatagacaagctcaacccggtcatccggggctgggccaactatcatcggccgatagtcgccaa |
| | | gcagaccttcaattacgtggattaccggatatggaagttgttgtggcggtggtgcaggggcgacatggaaatcgctgcaaac |
| | | gttggatcaaggagaaatacttcaagcgcatcggtacgcgcagttgggtgttctccgggcggtatcccagcggcaaactggc |
| | | cacattgctgtacgccgacgacacaaccatacagcggcacaagaaaatcagagcggaggccaatccgtacgacccggaag |
| | | acgagatgtacttcgaggaaaggctggaatacgcctggcggagatcggacgaagggaaacggaaaacgctgaggctgtgg |
| | | ctaggccagagcaagcgctgcccgatgtgcaagcagttgatcacgttcgagacgggatggaacatccaccacatcatcaag |
| | | cggcacatgggcggaggcgacgagctggacaatctggtgctgttgcatcccaactgtcatcgacagctacacagtgcggca |
| | | ccggctctttcgattgagaaagggcttacaaaggcttgagccgtatgcggggaaactcgcacgtacggttcttaggggaggg |
| | | gtggccagcaatggtcgctccttacccgacagccctgtcg |
| 3167 | Azotobacter vinelandii | ctttttgctggtgcgcccagcatgggcgcactcctgcgggtgcaagtcccgccatgagctggtcacagcgaatgaagcgaag |
| | | cgcaactgcgtgagggcgaccgagcgtggggaggaagcgcggagcgtaaatcgtgagccgatgaacaagaaccgcata |
| | | gaaggcgctgccgagcagggcaagcgggccagtaaccgcgaagctcttgtgaccaaggcgaggcggcgtagatgcggc |
| | | ggttgtgcggtgaaggagtgcgttcttacctggggagatctcgcctcatgcctgaaagggcgacggtgtcgagccggagcga |
| | | gaagtcagcagaggtcgtagtagccgcagtagcggggccgcagaggccagatgcgaaccggtgaaggaccgaacgaga |
| | | ggaagtgttggaaacgatgtcgatgtcggaggccatgcaccagaagcccgcgtgcgcggggcggcatgccgcaggccag |
| | | ggtgaagccccggccaaggtatcccgtggtgaagccgaaggcccgcgacatgagatggaaggcacagggtcggcgctgc |
| | | tggaagcggcgctgacgcgagagaacctgcggcaagcgttcaagcgggtgcgagccaaccggggatcggcgggcgtgg |
| | | acggtctggacatcgaccagacggcgcgcaagctggtgaccgagtggcctgcgatccgggagcaattgctgcgggggac |
| | | gtaccggcccagtccggtacggcgggtgatgattccgaagccggatgggagccaacgagaattgggtattccgacggtgac |
| | | ggatagactgatccagcaggcgttgttgcaagtgctgcaactgctgcttgatccgagcttcagtgagcacagctacgggttcag |
| | | gcccggaagacgggcgcatgacgcggtgttggccgcgcaatcgttcgtgcagtcgggccggcggatagtggtggacgtgg |
| | | acctggagaaattcttcgaccgggtcaaccacgacattctgatcgaccgcctacgcaaacgcatcgacgacgcgggagtcat |
| | | ccggctgattcgtgcgtacctgagcgcggggatcatggatggggggggtcatcgagcgggaccaggggacgccgcaa |
| | | ggcgggccgctgtcgccgctgttggccaacgtcctgctcgacgaggtggaccgggcgctggagcggcggggccattgctt |
| | | cgtgcgctacgccgatgactgcaacgtgtacgtacgcagtcggcggggggcgagcgggtgatgaatctgctgcgcaagct |
| | | gtacggccggctcaagctgagggtcaacgaagccaagagcgcggtggccagtgcgttcggccgcaagttcctggggtatg |
| | | ccttctgggcagcgccgaagggacaggtcaagcgcaaagtggcggccaagccgttggcgacgttcaagcagcggatcag |
| | | gcaactgacgcggcgcagcggtgggcgcagcatggcgcaagtcgtgcaggagctgcgtccgtatgtgctgggctggaag |
| | | gcttacttcggactgtcgcagacaccgagagtctggcgttcgctgggcgaatggctgcggcatcggttgcgtgccgtccagct |
| | | caaacagtggaaacgcggcaagaccctgtttcgggaactgcgcgccctgggggccagccacgaggtggcgcaacggatc |
| | | gcggccaacagccgccgatggtggcgcaacagcggcaagctcctgaacagcgtgctcaacctggcgtggttcgaccggct |
| | | tggcctaccccgactcgcctgacctcaagctctcgaaccgcccggtgcggacccgcatgccgggtggtgtggcaggggcg |
| | | ctgccttgacggcagccccctatgccgatcatgccggaa |
| 3168 | Agrobacterium radiobacter | ttggacgagggtgtgcggcgaggcacccgcagcagtgtgggagccgagctgcacgaaagatgagggtaggtccctcgaa |
| | | gccgaccgtcaggggtaggtttcaaaccactgcaagctgcgacggtaaagagccgtggtggtgagcgttgcagaaaaggc |
| | | ggatgttagtccgtcaggtaaggttcagagagacgagcgcaagcgaaccgctgatgacgtgtcgaaaagtgcaagatgatgt |
| | | cgaaaccgggaggaattcgttagcccgggacgagtctggggggcgacctgatttctgcccagacggcatccggcatgaag |
| | | gcggcgtgactcttcaccaggctttcgtgtggaacgcgggaacctgtcgctccgatgttaagggagaaacccaagcaggcg |
| | | gtccctgcgagggtgagattaccgaagcggggcacaggggcggagcagcccgtagtagtgctgaaggtcctgtaatgggg |
| | | ctggagcgaaggggttgcgttgttcagctctggagcgtggacaaccggcgacgggaggatcctcgtggatagagcaaagcc |
| | | gtatacgatcccgaaaagggaggtgtgggaagcatataagagggttagagccaaccaaggagcggctggtatagatggcca |
| | | gacgattgcggacttcgaggctgatcttaggaacaacctgtacaagctctggaatcggctggcgtcgggcagctactttccgc |
| | | cgccggtgcggcgggtcgacatacccaagagcgatgggaagacgcgcccgctgggcatacctacggtcgccgatcgcgtt |
| | | gcgcagatggtggtcaaacgccatctggaaccggtggtggagcctgaattccatccggattcctatgggtatcggcccggca |
| | | aatcggcacttgatgcgatcagcgtggcacggcagcgatgctggcgttacaactgggttcttgatctcgatatcaaggctttcttt |
| | | gatagcattgaaccggacctgctgatgcgggctgtgcgcaagcacaccgattgcccgtgggtgcttttatacattgaacgatgg |
| | | ctgaaagcgccagtgcagatgccggacggaaatctcgtcgccagagaacgaggaacgccacagggggggtgatcagcc |
| | | ccctcttggcaagcctgtttcttcactacgcgtttgacatgtggatgtgtcggaatttcccggacattccgtttgaacggtatgctg |
| | | acgacgcgatctgtcattgtcggagcgaggatcaggcgatggcgcttcagaacgctctggacgcgcgttttactgactgtggg |
| | | ctgacgcttcatcctgacaagactaaaatcgtctactgcagggatgaaagtcggcgaggtactcatccggtctacaagttcgac |
| | | tttctcggctacactttccggccgagacttgtgagtaagaaggccgggggcatgggtgtttcattcggtcctgcggccagtccg |
| | | acggcgctcaaggcgatacggggcacgatccgtagttggtccttgcaccttcgcagtgacaaagctctcgatgatctggcacg |
| | | gatgttcaactcgtacatccgcggctggatcaattactatggtcggttctgtccttcagctctccaacctacgctttggagtgtcga |
| | | gcgatacttggctcgatgggcatcgggaaagtacaagtccttgcgtaggcataaacggcgatcccggcattggcttttgcgcat |
| | | cgcgcagcgccaacctcggctgttcgcccattggcctctgcttcatggatacggtcgaacaatgggagccggatgatgcgag |
| | | agtatcacgtccggttctaagagagcgtgggggtggaactcccccgcgccactcgcctcctgatttc |
| 3169 | Alistipes shahii | aagtatttgtgtgcgcccgtacaagctgcttagtaaatatcccactggcaagggattaggtaagtgttctttgaaaaacctatcatc |
| | | acccgacttatccgaaagggaaaccggacggggagtgtagcatgtcgggaacgtcgtaagtcagtcagttaccagactgcc |
| | | actgaacggcaagatgaaacgatggatatgaggatgaaaaccgaattgtttgaacgacagtccgagctgctctacttgaaggc |
| | | tatgacgtaaggtaactgaaatcgtcatactgcggtactgctccggtgtataggtacggacgcaagcaagaacttgccgcaga |
| | | gcaaccatgataaatggtaaaggacggaaatcgcatccgacaatccatcatgccaacagttactaagtaagtaaacggggatt |
| | | acctaaaccggaacgccgtaaggctatgaggtgcagacctctgaatatccggcacggtaacggagcctccgtagtagtccgg |
| | | gcagggtaacgccctgcacatggcgaagggaggcagctaattttctttaatacaatttaatggataatgtgagagacattatgag |
| | | aagtccggaaagagtattaaacagtctgaacgaacacagtaaggattcgagctacaagttcgaacgcctttaccggattctgtt |
| | | caacgaagagttgttctatgttgcctaccaaaagatcgcatcgaacggaggcagtacgaccaaaggctccgacggtcgcagt |
| | | atcgacgagatgagccttgcccggatcgaaacgctgatagcctcgttgaaagacgaaagttatcagcctcacccgtcgcgga |
| | | gggtgcatatcccgaagaaaaacgggaaaacgcgccctctgggaataccggcttttgaagacaagctggtgcaggaggtag |
| | | tccgcatgattctggaagccatttacgagggacattttgagaccacctcgcacggtttccgacctaaacggagctgtcacactg |
| | | cattacttcatattcaaaagacgttcagcggggcgaaatggtttatcgagggcgacatcaaagggttcttcgacaacattgacca |
| | | tgatgtacttgtcggaatcctgcgggagcgcatttcagatgaccgttttatccgtctgatacggaaattcctgaaagcgggatac |
| | | gtcgaggattggacgtttcacaacacttacagcggaatgccgcagggaggtatcgtcagcccgattctggcgaatatatacctc |
| | | gataaattagacaagtacgtgaaagagtatatccgacatttcgacatgggaaccaaacgcaggccgggcaaagagagcaac |
| | | gatttggccaatgaacgaaaacggactgtgcggaaactgaaaaagataaaagacgggactgagaaggcggctttggtcgca |
| | | agactcaaagccatcgaacaggaacgtgcagcatttccaagcggagatgaaatggacggaagttaccgcaggctcaaatac |
| | | atccgttacgccgacgattttattctgggtgtaatcggtagcaaagaggatgcactgcggataaaggaggatattaaatcattcct |
| | | atccgaaagcctcgccctcgaactgtccgaagaaaagacgctgataacccatacgggtaaatcggcgaaatttctcggatatg |
| | | agattacggtaacacggaacaatcatcaacgacgggatgtgcaaggacgtctgcgacgcacctacggcaagcgtgtccggc |
| | | tgaatgtcagcatggcaacgctgcgggacaaacttctggaatacggagctatggaaatcaagctccgcaacgggaaagagat |
| | | ttggaaacccaaatgccgttcaggattgatattcaacgacgatcttgaaatcctcgaccgatacaatcgggaaacagtgggattt |
| | | tgcaactattacctgatcgccaacaactgcgtcgtactgcacaacttcagatatatcatggagtacagcatgtataaaacatttgc |
| | | gggcaaatataggagcacggtacgaaaaatcaacaaaaagtaccgtctcaacaaactgttcaccgtaaagtacgagcagaaa |
| | | ggggtaatcaagtcccgaaccttttacaagacaagtttcaaacgccggacaacggcgttcaacggaagctgcgacatcgaac |
| | | cgtactctatcgcagacgcgagccgaaccaatttgacagacaggctcaaagcggaaaaatgcgaattgtgtggggcaacgg |
| | | gcaagctgattatgcaccatgtccgcaacctcaaagacctgaaagagaaagagagttggaaacggctcatgtcagcccgaaa |
| | | acgcaagaccattgcgttgtgtccgagttgccacaggctgcggcatctgggaaaagtttagactgaaaaaaaaaattagtgga |
| | | gagccggatacgccgagaggtgtaagtccggttcgggggcgagttcttggaaacctgccgtagaaatacggtaaggcgccg |
| | | ggtgcttagcctacataaaacctg |
| 3170 | Wolbachia sp | tcagtaccaagtgcgtccagagaaggcgagtattgttcagtaggaaatagacctaccaggataaaagtcggaatcctgtagctt |
| | | gggtagcagcataaagggaaaccaaaatgttgaagcctaccgacaacattatcgtaggataatggcgaattaccaggccgta |
| | | gcgaaagcgaatgcacagattgcctcgaaaggaatattctgaaggtcgagccatcttggtactggcgaagacagaatagacc |
| | | agacgaaaaacgaatgaaacgattggtctacttcagcggggtgttagtaacagcatggcaatgaggacatacaaagcaactg |
| | | gagaagccctcctcatcccaaggagaaacaccttggagaggtaggttctataactggaaaacaggaagtgaactgaagatga |
| | | gagggtgacggatgggtgcgtagtagtgaagaagtgaggtaacgctcatggagcaaaggcaccctactagcagggatctttt |
| | | aacaaaaggaaggtaaaggtaaaaatgacaaaaatgccgataaaattacaagacctgagaagaaagatatacaccaaagcg |
| | | aaggcagaaccggaatggcgattctggggaatccatgttcatgtctgtaaaatggagacactggaggaagcttacaaactaac |
| | | gaagaggagtaatggggcaccagggattgatggggtgacattcgaatcgatagagacagagggctcaagaaagtatcttca |
| | | acgaataagacacgaattaataaccaagacctatagcccaaataggaaccggagaaaggaaataccaaaatctggagaaaa |
| | | attcagaacgttgaacataccctgcatccgggatagaatagtgcaaacagcactgaagctaatacttgaaccgatattcgaatc |
| | | agacttccaaaaaggatcatacgggtatagacctaagcgtaatgcgcatgaagcagtacaaaaagtaacggaagcagcaatc |
| | | aaaggcaatacaaaagtaattgacgtagatctaaaatcctactttgacagtgtgcgacatcatattctgatggaaaagattgccaa |
| | | aagaataaatgacaaagaaatcatgcgcatgattaagctaatcctcaaaataggaggaaaacgaggaatggcacaaggatca |
| | | ccactttcaccactactaagcaatatatacctaaatgaggtagataaaatgttggagaaagcaaaagaggtgaccaaagaagg |
| | | aaagtaccaacgtatggaatatgccagatgggcagatgatttggtgatactgatcagagaatacccgaaacgggaatggctag |
| | | aaagagcagtatacagaaggttggaagaagaattagcaaaactagaagtgagagtaaatgaagaaaagacaaaagtcattaa |
| | | cctaaagaaaggagaaacatttagcttcctagggtttgattttcaagagaatatcacaaaacaaggaaagtggaatgtcaggaa |
| | | aacaccgcagatgaaagcacgaacaaacctgctacgcaagttgaaggaaatatttcggcgccataaatcgcaaccgataaaa |
| | | agagtgataaatgacatcaatccgatattgagaggggggtaaactactttaggataggaaactctagtcgaaagttcagttacg |
| | | taaaacactgggtggaaaagaaggtaagacgcaatttaatgcgagccaggaaagcaaagaaagggtttggttggaagaaat |
| | | ggagtagcgaatggatatacaaaacgctgggtctatattcagactacagaataaggtattacagaccgaaagcatcaccaaca |
| | | caatatgtgataaactttggtaaagaacttgctaggaaagcgtagtgcgggaaaaccgcatgctgcgtttgacgaggcgggag |
| | | ctggaaacgttaacatgggaatttggattgaggcccaggatgaaaataatggatcagccaccaaaccctaaagaggcgcgcc |
| | | agttctcgaccctacagatgcgctt |
| 3171 | Xylella fastidiosa | tggcaagctcgtgcgacgtgaagtcgcttgtttcattgcttcaccgcttcggcgtgatgaagcctgatgttccgtcatcagtagcc |
| | | tatcggtgcatgcgtggttggcaacgactgcgtgtgaagccaccgagacaatgtaggtcggcgaaagccaggtggagaccg |
| | | cgaggtcgaaaccgcatctgcgagcgccaacgcggatggatcgctagactggtcggtatggttaacgtatgtgaagtgctgat |
| | | aaacaccgtcaagatgaaagagccaaagacgctgacaggctctacccaaaatggggcgtagctggacattcctctgagcttt |
| | | gggaatgcacggacacgaaagctaccggtggagaggcgcaacctaacctgacctgtgaatcaagtggaacacggtaagcc |
| | | cgtatctctgcccctcggggcagacagaccgtaaggaaagttcatggagatgcgggtagaggaatgtggaaaaagcgaatg |
| | | ccgccctgtaatggggcggataggggctgagccgcgaggcaacatcgcttcacgtgaaaacgggcagacttccacttggtct |
| | | ttctttacgagattgtttgaagaacctttttaggagagaaagcaaatgactacgcaagctgcttgcgcgggtgcgctctccggcg |
| | | actcgggcggatggcactgcatcgactgggccaactgccaccgggaagtcaggaggctgcaaacgcgcatcgtaaaggcg |
| | | acacaggaaggccgctggggcaaggtgaaagccttgcaatggctgctgacccactcgttcagcggcaaggctcttgccgtg |
| | | cggcgggtgactgaaaaccagggcaagaagactccgggtgtggacaaggtggtatggggcaccccggaaaagaaggaa |
| | | aacgcagtagatgagttgcggcgccatggccataaagccagaccgctgaggcgagtctatatcccgaagtccaatgggaag |
| | | ctacggccattgggtatcccaaccatgagagatcgcgcgatgcaggcattgcatctgctaagtctgctcccggtgtcggaaac |
| | | ccaagctgatggatgttcctacggcttccggcccgagcgttcagtggcagatgcagtcgataggtgcttcagggcgcttaatg |
| | | gacaacattccgcccaatggatactggaggccgatatcaagggatgcttcgatcacatcagccacgactggcttctgggcaac |
| | | gtgcccatggacaagcggatgctggaaatgtggttgaagtgcggcttcatggaagcaggtgcatggtcggaaactgaggca |
| | | gggacaccacaaggcgggatcatctcgccgacactggcaaacatggcgctggacgggctggaacaactattggaacagtc |
| | | cttccgctcaagaatgcgagatcggcagatcaccaacccgaaggtcaaccttgtccgttatgccgatgactttgtgatcaccgg |
| | | agactcagagaagcttttgatcaaggaggtcaagccactggtggagcgattcctggcggaccgtggactcaccttgtctgcgg |
| | | agaaaaccagggtgacgcacatcgacgaaggcttcgacttcctcggccagaatgtgcgcaagtacaaagggaagttgctgat |
| | | caagccatcagcagcgagcctcaagaattgcgcagacaaaatccgggccatcgtacggtcgaacaaggcagccaatcagg |
| | | catcgctaattcacctgctcaaccctgtgatccagggggggcaaacttccatcggcatattgttgccaagaagaccttcaagtc |
| | | tttggatgatcatgtctggagggccctgtggaaatgggctctacggcgtcatccggcaaaaggcaaaaagtggattcggcatc |
| | | ggtatttccatgcgtagcgtggtcgtcaatgggtatttgcagcccgatacgtggactcttcgagcaaagaggctcgatgggcaa |
| | | ccctccgtaaggcagtagacaccaagatcaggcgaaatacgatgatccgcatgggtgcgaatccattcgacccggagtggg |
| | | aaacctactttgaacagcgcctcggcgccaagatgaaggacaacctacaaggtcgaaaacgtctattgcacttgtggttggaa |
| | | caggacgggaagtgtcctgtatgcgacgaaccgctgacgaaagaaagcggctggcatgtccaccatatcgtccgtcgcgtc |
| | | gatggtggttccaatcaaagcgggaacctggtgatggttcacctgaactgtcattaccagatccacaaccttggtttggatgttgt |
| | | gaaaccggctccagcaatggggctttgaaaggcttgagccgtatgaggggaaaccttcacgtacggttcttaggggaggatg |
| | | tggtggcgacgccatgtccttacccgaccacataggca |
| 3172 | Zunongwangia profunda | ggatgaaagtgtgtgccttgaagcggcctaaagtaatgccgcaggctgttagattgatggtggtagacccaccgtaggggttg |
| | | acagacagaccctacaaaccaccttgtggtgctttatttcgaaagaatgaggtgctgagcgacaaggaaaaggcgagagaat |
| | | actcgtcaggtgaatttcagtgagctgaacgaaagtgaatcactggttaagttaaagtaccgatatcgccagattctgtcgaaat |
| | | cggagaattgtgtttcttctccgagaataaaacttagtggcagtaacctgaagttgactaagtgacaggcggtacagaggtggc |
| | | aggacactgaaataggcgatctaacggaacaagggaatttgtacagggatggtaagggaaatgcacaagcggaaaccccgt |
| | | gaggcagaaaaccaatgaactgtacagaggcggactaagccgtagtactgcagaagtctctgtaatggagatggagggaag |
| | | ggcttagccagattagttttatgtaagacacaactttgaaagaaggatgattgattattatgaaacaaaacaacatccaataacca |
| | | agaaaatggtattggatgcgtacaagaaagtaaagtccaaaaagggcagtgccggagtcgatggacagagtttacagaatttt |
| | | agggagaacctctctggaaatttgtataaaatctggaaccgtatgacctcaggcagttattttccgcctgttgtaaaggaagttcg |
| | | tataaccaagaaaacaggaggctttcgcagtctcggaattcctacggtatcggatcgcattgcgcaacaggtgattaaaagtta |
| | | cttagaacccaaggtggaaagcagttttcatcagaatagttatggatacagacccaggaaaagtgcgcatcaggcactggaga |
| | | aaacagtgtccagatgtggttattacagttgggtggtagaccttgatattcgtggattctttgataacattgaccataccctactgat |
| | | gaaggcagtagaaaggtatacaaaagagaaatgggtgttgatgtatattggaaggtggttgaaaacaggagtatccagagaa |
| | | ggtgaaatcaccgatagaataaaaggtactcctcaaggcggcgtcattagtccgctacttgccaatatatttcttcactttgcattc |
| | | gataaatggatgcaaatccatcactccaatatgcctttcgaacgatattgcgacgatgccatcatacattgcacatcagagaaac |
| | | aagcatatttcattagagaggctgtttctaaacggatgaaagcttgtaagttggaactcaatagtgagaaaactcacattgtgtatt |
| | | gcaagaatcacgtgcacagcgaaagtcacaagaatacaagttttgattttctcggttacacgttccgccccctcagacgaccaa |
| | | ccaaaaatggttggaaactgacttactttccagtaatgagccacgcctcaaagaaagaagcaaggcgaagacttaagaaggtt |
| | | gtcaatagaaagtttcggggatccatccaagaactggcacagataatcaaccctatgataaggggctggtatcaatatttttgca |
| | | agtattcgaaatggacaacccgtggactatggtattggttaaacaggaaaatagttagatggattaggaggtacagaaagcgc |
| | | agtagagtgcaagctcgaaaatggctaaaaaatgtgtacaagactaacccacaattgtttgagcattggaaacctgtcttagata |
| | | taaaaccttactaattgtaccgttaatctggaagcgccgtatgatgggagactatcacgtacggtgctgtgagagacttggggtg |
| | | aaattccccttgtctactcgacccgataaagg |
| 3173 | Zunongwangia profunda | acttgcgatagtgtgccgagcaaacaatgaccggcaataaatagtcattgtgaactataattaagatggtagaatcgacctacc |
| | | ggtgtcgtcttataggagaaggcatcaaaccaaccaaagatgctttggtaaagagccaaggtattgagcgtttaggaaacggc |
| | | cagtcaagaattggtcaggtatgattaagagagatgaacaaaagtgaaccccttctgaggtgtcgagaagttgctacatcctgt |
| | | caaaagccacgaagtatcgcacggggttaaagagtatagcagttacctatttattggctatacggcaggcgtcattcaggggg |
| | | catgactcttattcaggcttagttatggaactcgggaacctgactatagatgctaagggaaatacacaataggctcaacctagag |
| | | gtagaataccaatgctgttttcagggacggactaacccgtagtagcaatgaagttcctgtaatgggaatagagcaaaggggtta |
| | | ggttatacagttacgttacatttaacaactcagttttgaggatgactttatgcaatgaaacaaaatcaatacctataagcaaagctat |
| | | gatatggcaagcttataaaagagtgcgcgccaacaaaggaagtgcagggatcgatacagtaagcattgagcaattcgatgag |
| | | agtttatcaaagaacctgtacaaactttggaatcgtatggcatcgggaagttatttcccccctgcggtcaaagaagtggaaatcc |
| | | cgaaaaaggatggtaaagtccgtaaattagggattcctacgataagtgaccgtatcgggcaaatggtggtaaaaatgtacctg |
| | | gagccccgtttagaaaatgtattcaatccaaactcctacggatatagaccaaacaaaagtgcccatcaggcacttgaacaggtc |
| | | aggaaaaactgttggaaaatggattgggttattgatctggacattaaaggtttttttgataacattgaccatcataaaatgatgcttg |
| | | ctatagaaaaacacgttcctgaaagatgggttaggttatatatagcacgttggctcgcaagtccagtaatgacaaagtcgggaa |
| | | atttagtctctaaccagggaagaggaactccccagggaggggttataagcccattactggccaaccttttccttcactatggttta |
| | | gataaatggctggaacagaacgacaatacggtgaaattcacacggtatgccgatgatgtgattgtgaactgtaaaagtcaaaa |
| | | gcacgcagagcaaacacttgaagctatcaaaagcaggatgcatcaaattggtctggagttacatccagagaaaaccaaaattg |
| | | tatactgtagagattaccgaaggcaagaaaagtattccaatgtgaagtttgattttctaggttattcttatcaacctagaactacaaa |
| | | atcgaagaaatcaaacggactctatcttggctttgattgcggcataagtataagttcgaggaaacgaattgcagataaacttgaa |
| | | gaattgaaagtagagcgaatgacttctgatcgtatagtaggaattgctgccattctcaatccaatgattaggggatgggtgaact |
| | | actatggtaaatttagaaggtcaatgctacacaaagtcttcaagttattgaataatcgaatcgtgaaatgggcaagaaaaaggtat |
| | | aaacgttacaaaaccagtataaaacgtgcctatcaatggtttgaaaggattaaggaacaatatccaaaactattttatcattggca |
| | | ggtgggatttgtatggtaatttgtaatttagattgtataacaagagccgtatgatgggagactatcacgtacggttctgtgagagg |
| | | cttggggtgaaattccccttgtctactcgacaccgtaaccg |
| 3174 | Vibrio vulniricus | tcaagtaaaagtgtgccgtgggcacaggatcctgttatctaaggaaggtaaacaggagaatgagcataagctcatgaagagat |
| | | ggaggtaggccctccagagtctgctgtcggaagtcggctctaaaccactttcaagtggctgttattaagagtaatggtcagaag |
| | | cgttgaaggaaagtcagaatagaattctggcaaggtagagtgcaaggagatgagtaatcccgaacccatgtggacgcgtcgt |
| | | taattagaaccagcatcaaaactgaggtcgttttattatctcaggacgagtacaccggtaaattccgaatgctgggtgtgcggtg |
| | | ctcggcgtaaagtgggcatgactttgcactaggctctttgtgggaaccacgggaaccagtcatcacgatgtaaaggaagaaat |
| | | ataaatgactaaattcatgagtatgagagtaccgatgcgtgatactggggcggagcaattcgtagtagtgatgaagggcttgta |
| | | atgagcgtggagcgaagggattgcgtcaagtcggttggaacgatgactcaactgccgacaggcaggaggaggtcgcacgg |
| | | acaaccaaaaccttttgcaatatccaaatgggatgtaatgactgcgttcgagaaggtaaaagccaacaaaggcggagccgga |
| | | gtggatggagtaacgatagaggactttgaaaaagaccttaaaaacaacctttacaagatatggaacagaatgtcatcggggtc |
| | | ctactttcccacaccggtcgcagcggtaagtataccgaaaaagtctggtggagagagggtattgggtatcccaacggtcagcg |
| | | accgagtggcgcaaactgtggtaagagacaagcttgaaatcatgctcgagcatcacttcctagatgactcttacggctatcgag |
| | | tgggtaaatctgctcatgatgcgatagaagtcactagaagacgatgttggcagtatgattgggtactagagtttgatatcaaaggt |
| | | ctctttgacaatattcgtcatgacttgttgatgaaagcggtaaagaaacatgttcagctcgctgaagagagtcagagccgggatt |
| | | atcaatggataacactgtacatcgaaaggtggttagttgctccgttacagaaagcagatgggacgcaaacagaaagagagtta |
| | | ggaacgccacaaggtggcgtggtaagcccagtgcttgccaacctatttcttcactatgtttttgataaatggctggagaagaatta |
| | | tccagacaacccatggtgtcgatacgcagatgatggacttgtccatgcaaggacaaagccgaaagcggagaagttgaggga |
| | | tgagctagcgaagcgcttcaaggagtgcggactggagatgcacccaattaagacgaagattgtttactgcaaagatgatatta |
| | | gacgagggtcaggtaagcatatagagcataaacaatttgattttctagggtataccttcagggccagaacaaataagtgtaaac |
| | | gtacaggtcaactctataatcgattcctgcctgcggtcagtatggcagcaaagaaagtcatgcgaaggcaaatcagagagcta |
| | | agagttcgccaggaaacgcagtacagcttagagcaattaagcagatggttaagtccaatgctgaatggttggataaactactac |
| | | ggaaagttcaggcgaagcgaattagactcggtattcagacacttcaacaagactttagtacgttgggcgagaaggaaattcaa |
| | | atcgctaaaatgtcacaaaagtcgaaccgtagcgttctttgataagctatcggctcaatgccctagattgtttcctcactggaaatt |
| | | tggttcagcaagaagttttacttgatgggagccgtatgagctgagaggttcacgtacggttctgcgagaggctgctggggtggt |
| | | tccggtggcctactcacctcagttggtt |
| 3175 | unknown marine bacterium | tgagattatgtgtgcacagaggattgagcgagagtaaagcacatttaccccacataaattgctgaatcgaactatagataagatg |
| | | gtggactgtaagccatattttaaagtttatttaaatagaagattaaytgtagaggtcgagcgaacagactggctgagttagtcaat |
| | | aaatagctttaaattacaggtaaaaaaaaacaggattgaaccaccagcagggactgtcaagagtatctgctaaaccgccttacg |
| | | gtgcttttatttaacaatagaggtattgagcggctaaggaaaaggggcgatgaaagaatcgtttcaggtgtttgtacaagagctg |
| | | aacgaaagtaaactgaccgatgagatgtcgaaactcatttaggcagtgttggaaaccgagggaatataatctgcttcgggaca |
| | | agcacaagggtaacttgaatataccacttgagccaacactcggcgtataggcagcaggcttgtatatgggcttgtttatggaact |
| | | gtggaaaccatcatatgttgtaaattgaaacaacacaagcggtaaaaccrcgaggttaaaagtagagatacatatgatggtggc |
| | | ggattaagtcgtagtagtgaagaagttcttgtaatgagggtggagcgaaggacttaagcaaacaggagttaacaattgcaaca |
| | | acttaaaattaaggatgattgaggcgttaagaccaaaaacacaaccagtaagtaaagagcaggtatggtctgcgtggaaacga |
| | | gtaaagcgaggaggaaaaggcatgggagttgaccatgttagtatggaagccatcgcatcgaatccacggaaatacctgtatc |
| | | cattgtggaatagactatcaagtgggagttattttcctccacctgttaagttagtgcctattcccaaaggagatggcaaagaaagg |
| | | atgttgggcataccaaccataatcgacagagtagcacaagaagtcatcaaggcggaattggaagtcatagtcgaaccgaggtt |
| | | tcaccccagttcatttggctatagaccgcacaaaagtgctcatgaagccttggaacaatgtgcaaagaatagttgggaaaggtg |
| | | gtatgtggtagatttggacattaaaggattctttgacaacatagaccatgaaaagatgatggggatattacgaaagcataccaac |
| | | aagaaacatatcctactttactgcgataggtggctaaaaacccctatgcaagacagggttggaggggtacaggcaagaatgaa |
| | | aggaacgccacaaggaggtgtaataagccctttgttggcaaatctttatttgcacgaggcttttgaccaatggatatcgacaaca |
| | | caacctcgtatagtatttgaaagatatgccgatgatattgttattcatacaagaagtatggagcaatcgcattttatattagacaaatt |
| | | aaaagcacggttgaaaagttattctttagagctacatcccgataagacaaagatagtatattgctatcgcactgcgcggtttcaca |
| | | aagaaggtaaagagatacccgtatcttttgatttccttggttttacttttaaaccacggctttgtttaaaatcaaacggtgagaaatttt |
| | | ggggattccgtcctgcaattagtaaaaagagtgagaaacggatattaggagagttacgaaagttaaaaatacatagatggatat |
| | | atatgaatattcatcaagtatcacaacgcttaagagcaaaggtacgaggttggctaaattattatggtagatttcgtaaatcagag |
| | | cttagttggattttcaggcatctaaacatccgaatttcaaaatgggcacggaataaatataagttaaagacttattccaaatcttatg |
| | | ggtggcttaaacgtatggtaaagtggtatccaaatacatctttacattgggagcatggctttacaggttaggtttgtttggaagagc |
| | | cgtatgaggggagactttcacgtacggttctgtgagaggtttagggtgaaatcccctttacctacttgactcgcttgtgt |
| 3176 | Crocosphaera watsonii | ttatacttctgtgcgattcgttgagctaaaaagctgacatccaagtacatcacaaggatacagcgtgtagctcattcaagagcctt |
| | | ctaaaggcaattcaatggttaaacgaccaatctaccccaaagatgaaaaacaagagccttaaagctcaattttcaaggggcag |
| | | aacacaaaggtcagaagggtaaaaacaatacccgaacggtaaaaaccaaagaaaagtttacaaacttaagtagtaattaccg |
| | | aattaactaagtagggataaccgaacagtaaacgaacctagaaacttgaataactgttggtttgttggctaaaggagccatactc |
| | | ggaatcaagggagaaatcccgagagtaacgacataaccctccgttttggacttagtaaacccaggacggaagcagggataa |
| | | aaggggatacccactgtgaaacagcacaagaaatcctcaagtcaatcggtcacggttaaacattacgttaaatcatcgacttga |
| | | accctcaaaaaaccgaagtagccaaatctgtttgataggctgtgttcaaaagaacaaggtgaaaagtggagtttttgaagtaac |
| | | atccgcacagattcctaaaagtaacctgggggagagatgaaacctaacaccgatatacccaaaccaacggaacgaattaagt |
| | | ccatgattgctcttaccactggtcggtaggtaagtaggaacaaacttaaccgcaagggtcatggatgtgagaagcaataagaa |
| | | gctaatgcctacttgtaataagtatggatatgctgacgtattgctggtctattagtaagaaatagtctttattagaggtagttaatgtc |
| | | gaatgcgagtataaccaagactacgccagaatggaacacaatcaactgggcaaaagtccagaggaaagtgtttaagcttcaa |
| | | aagagaatatttcaagctgttaaatcaggtaataaagttaaagctaaaaagcttcagaagttactgttaaagtcacactacgcaaa |
| | | gctcttagccatacgcaaagtgacccaagacaaccaaggcaagaaaactgccggagttgatgggaaaaaggcactacaccc |
| | | taaccaaagaataaagttagtcaaagaattaactttaaaaggttacaaagcaaaggcactcagacgggtatggattcccaacca |
| | | cagaagagatgaaaaacgtggattggggatacccaccatgaaagatagagtaatgcaagccttggttaaatcagccttagaac |
| | | catattgggaagcccaatttgagggaacatcctatggttttcgtccaggcagaagcgcacacgatgctatcaccagaatataca |
| | | cagcaattaataagaaagctaggtatgtactagatgccgatatcgccaagtgctttgacaagattaaccatgattacctactgtct |
| | | aaagtagattgtccacacaacatcaaaagaatcattaaacaatggttagaatgcggcgttatggacaaaggcatcttagaggaa |
| | | accgattcgggaacacctcaaggtggtgtaataagcccactattggctaacattgcactacacggaatgataaaagatacaga |
| | | aaaccacttcccccgtacaaaacgtaaggaagatggaagtctacacagggagtataaacccaaaatcatccgttatgccgatg |
| | | atttcgtagttctccacgaggattacgacgttattctacaatgtaaaactttaattgcacaatggctagaaaaggtcggattggaac |
| | | tcaaaccagaaaaaacctccatcagacataccctaaaaagtattgaacatgacgggaaaaccattgaccccggtttcgatttcct |
| | | tggattcaacataaggtcataccccgttggaaaacaccactccggtaaaacaggaggaaattcaaaatgggggatagaatcca |
| | | aagcaataggtttcaaaaccttaatcaaaccaagcaagaaaaagatactggcgcatcatgaggcaatcaaagaggttgtaaaa |
| | | gccaacaagaaagccccacaagaagtgttaatcgcaagattaaaccccataatccgaggatggtgtaattattaccgaactgta |
| | | acaagtagagaaacattctcatccgaagattcaatattatggaatacacttagagcctggacagtaaacaggaaaaagaaaga |
| | | gacaccactgtatgatgccctcaaaaaatatttctcatacggaaaacaagggaaatggacattccaaacaagggaatatgtcct |
| | | ctatcaccacaccgaaacggaaattaagagacatacactggttaagcctgaatcatcaccctacgacgggaactggacttact |
| | | ggagcaaaagaagaggtacatatactgggacaccagccagggttgcgaaactcttaaagaaacagaaaggtatatgccctca |
| | | atgcaagcaacacttcacacctgaagacttgatagaagttgaccacatcatcccgaaatcgaaaggtggaaaggatacttaca |
| | | acaacctacaagctctacatcgtcactgtcacgatgccaaaagcaaaaatgactacctctatgattggcacttataatggctatga |
| | | atggaaagacgatgtattaacagtaccctagacaagggactaattgccgagagccgtgtgaggtgaaagtctcacgcacggtt |
| | | cggaatgggaggagaggaaggtgacttccttttcgacccctaatcatctatccc |
| 3177 | Clostridium cf | gaaatttaccgcgcgtccagaagcgccgcttgtcatccgcaggcggtgtgcggacattttgggaatccggctttggcaagccg |
| | | gtaaagaaaagtatcaaagacgctgaaaaagtgtcgctttagcgtcagctggtttaccccggagggaaaccacaagggggac |
| | | aatagcgtaccggaaacgtcgcaagttgggaaaaccatctacccacgactgagcggcaagacgcaacattgtgaacaagga |
| | | ataaggctaaactgctttaagggcagtccgaggcgggatactcgacccggggcgtaggatggttgtattcgccgtatgtcat |
| | | agcggaatgtgacaggtatacacagaccgcaggatacttatgacagccagccgcagtaagcggaaaaggacgaaaaccca |
| | | ctccgacatcacaatacgcttttcccaaagtgtctaactggagattgcctaaaccggaacgccggaaccatccggctatgcgta |
| | | atgccgcaaggtgaaaaatttcaaggggtaaaaacctaagaaagatgacgctgaatatccggcatggcaacggagccttcgt |
| | | agtagtccgaggacgggaaagccgtctacatggcgaaggaaggcaggatgtcaaccaactcataaaaaggaaaggtgcgt |
| | | gaggcattatgagaagtcctgagaatgtgttggaaagcctaaaatccaaggcgtgtaaccagagttacaagtacgagcggtta |
| | | taccgcaatctgtacaatccacaattctatctgctggcataccagcggatacaggcaaaaccaggcaatatgactgccggaac |
| | | agacggcaagacgattgacggcatgggaatggcgagaatcaacgccctcattgaaaaaatgcggaattccagctaccagcc |
| | | caaccccgcaaggagaacatacattccgaaatccaacggaaaaatgcgccccttgggaataccgtcattcgatgacaaactg |
| | | atacaggaggtggtgcggctcattctggagagtatctatgagccaaccttcagcgaccactcccacggcttccgcatgaacaa |
| | | aagctgccatacggcactcaagtatgtgcagaaatatttcacggggacaaaatggtttgttgagggcgacataaagggctgtttt |
| | | gacaatgtagaccatcacgtgctgattgccattctgcgaaagcggattgcggacgagcagttcattggtttgctctggaagttcc |
| | | tgaaagccggatatatggaggactggaactaccacaatacttactccggtactccgcaaggctctattatcagccccatcctcg |
| | | caaacatttatctgaacgaattggatcacttcatggcggaatacgcagagaagttcaactgcggagatcgccgtagaatcaatc |
| | | cggcgttcaagaagaaattggatgtctgccgggggaaagaagaacggctcaaaagaaacatctccaaaatgagcgaggag |
| | | gaaaaagagggcttgctcgccgaaatcagcgaactgcggcgtagcttgcggtcaatgccgtacagcgaccagatggacgaa |
| | | ggctataaaagagttttctatatccggtacgccgatgattttctgattggagtaattggcagaaaagcggacgcagaacaggtca |
| | | aacaggatgtggggcactttatccgggaaaatctccatctggaaatgtccgaggaaaaaactctgattacccacgggcatgac |
| | | tttgccaagttcctgggatatgaggtcacaatcgccaaaggtgagtgtaacaaaaagaccagaaccggagccaccagacga |
| | | gtaaacaacggaaaggtcctgctctatgtcccccacgacaagtggatcaagcggctactctcctatcatgccctcaaaatcaga |
| | | tacgacaaacagaatggaaacagagaggtttgggaacctgtccggcgcacccgcctgttgcacctggacgatttggaaatcc |
| | | tgaaccagtacaacgcagaaatccgtggattgtacaactactatcggctcgcaaacaatgtgtctgtcctcaacaacttctactat |
| | | gtaatgagatacagtatgctcaagaccttttccggaaaataccggacacgaattagcagaatcattcagaagtaccgtcaaggg |
| | | aaagattttgttgtggagtatccgaagaaaaacggccaggttggaaaggtgctgttctacaacaacgggtttcgccgtgatacc |
| | | aaagtggaaagcggcaatcccgatgtgattgcaagagtgtttgagaactacggacgcaatagcctgataaagcggctgaaag |
| | | cgagtcgatgtgagtggtgcggtgcagagaatgtaccgattgaaatacaccatgtacgaaaactcaaagacttaagcggcaa |
| | | gaaacaatgggaaattgccatgattgggcgcaagcgcaagacaatggcactctgcgttgactgtcacgataagttacacgctg |
| | | ggaaattagactgacatctggagagccggatacatcgagaggtgtaagtccggttcggagggaagttctcggaaacctgcca |
| | | tagtgatatggcaaggcgctgggttcttaccctacttgtcacgtc |
| 3178 | Clostridium saccharolyticum | gcttttttccgtgcgcccagcatgggcgcactcttggaggtgcaagtcctcccgtgagttgatcacagcgaacgaagcgaagc |
| | | gcaactgcatgagggtgaccgagtgtgggaaggaagcgtggagcgcaactgcgagccgatggacaagaaccggatagaa |
| | | ggcggtgccgagcagggcgagcgggcagaaaaccgcgaagctctcgtgatcaaggcgaggtgtcgtaaatccggcggtt |
| | | gtgcagtgaaggagtgcgttcttacctggggagatctcgccttgtgcctgaaagggcgacgtcgagaggcggagcgagaag |
| | | tcagccgaggtcgtagtagtcaggggctgggccggtgaggctgaaaccgctggcgaaggaccgaacgagagggagtgta |
| | | gaccgacatgtcgatgcggcaggccatgcgtcagatgcccgcgcaagcggggcgggcgggagtagcgcgcggtgaagc |
| | | cgcgcgtgtacccgtcagcgacgaagcctgcggcccgcggcgtgagccggaaaacacagggtcggcgctgctgcaggca |
| | | gcgctgacgagagagaacctgcaacgggcgctcaaacgggtacgtgccaataaaggcgcagcgggcgtcgacggtctgg |
| | | acatcgaccaaacggcacgccatctcgcgacggcctggcccggtatccgtgagcacctgttgcgggggacgtaccggccg |
| | | agtccggtacgacgggtagcgatccctaagccggacggcggcgagcgcgagcttggcatcccgacggtgacggatcggct |
| | | gatccagcaggcgctgctacaggtactgcaaccgatacttgatccgagcttcagcgagcacagccacggcttccggccggg |
| | | gcggcgtgcgcaggacgcggtgcttgccgcgcaggcgtacgtgcagtccggccggcgcgtggtggtggatgtggacctg |
| | | gagaagttcttcgaccgggtcaaccacgacatcctgatcgaccgcctacagaaacgcatcgatgacgccggggtgatccgg |
| | | ctggtgcgtgcgtatctgaacagcgggatcatgagggatggcgtggtgcaggagcgcgggcaggggacgccgcaaggcg |
| | | gaccgctgtcgccgctgctggccaatgtcttgctcgatgaggtggacaaggccttggagcggcgaggccactgcttcgtgcg |
| | | ctatgccgacgactgcaacgtgtatgtgcgcagtcgccgggcgggagcgcgggtgatggctttgctgcggcgtctgtacggc |
| | | aagttgtgcctgaaggtcaatgaagccaagagcgcggtagccagtgtgttcggccgcaagttcctgggctacagcctgtggg |
| | | tggcccggggaggaacggtcaagcgcaaggtggcggccaagccgctggcgacgttcaagcagcgggtgcggcaactga |
| | | cccggcgcctgggcgggcgcagcatgcaggacgtggtgcaaaggctgcgaccttatctgctgggttggaaagcgtacttcg |
| | | gactggcgcaaacaccgaggatctggcgcgagctggacgaggggttgcgtcatcggctgcgtgcgatccagctcaagcag |
| | | tggaaacgagggccgaccatgttccgggagttgcgcgcgttgggcgcaagccccgccgtggcgcagcgggtggcggcca |
| | | atagccgctgctggtggcgcaacagcgacaaactgctcaacagcgtgttgacggtggcatggttcgaccgcctgggtctacc |
| | | ccgtctgtcctgacctcaacctctcgaaccgcccggtgcggacccgcatgccgggtggtgtggcaggggcgcagtctacgat |
| | | gactgtcccctatgccgattccgcacagc |
| 3179 | Burkholderia cenocepacia | tacgtacagtgtgcgccgggagaccggtagagatcaaggggtgaaagtccccgacattgcaagaactagcgattcacgattt |
| | | ccccgagtcatgcgtggccagccgcgaggcgggttgcgaagcgttgacaggggcgctgataggccagccattgagccacg |
| | | aaatggatacattcccgagtgctgacgtactactgtatgcggaaggcaacatcgcgcggcgcgctatcgcgagcgctgcgcg |
| | | gactcggcgtggtctgagaccctggcatgtcagtacgatctctacgcgggaaccgggagatctcccatctggccgccgacgg |
| | | aaatatggtcgagcggtccgcatcgggaaggcgaggagctgaagccgatgacgaacggatgggagaagtcggactcgcc |
| | | catagtagcgaagaagctggcgaacaaacccgggcaaccggatgcggagtcagtggagcgaaggggtggggccaaggg |
| | | gaacacggaatggtcgcgcacgcgccggacgcagagccggataagcgtgtcccagagactggaccgtgtgcgtcaagcc |
| | | gcaaggcaaaggaagaaggagaagttcacggcgttgtaccacttgatcgacctcgaccttctggcgacggcgttcttctggct |
| | | gaagcggaaggcggcagccggggtcgatggcgtgacgtggcacgactatgagcaagatctcgaccgaaatctcgaagac |
| | | ctgcatggcagactccgccggcaggcttatcgggcgctgcccagtcgcagacggtacataccgaaggcggatggcaaaca |
| | | gcgaccgctgggcattgctgcgctggaagacaagatcgtccagcgtgcactggtggcagtactcaatgctgtctacgagatg |
| | | gacttcttgggattctcatacggtttccggccgcaacgcagccagcacgatgcgctggatgcacttgcgacgggaatcgctcg |
| | | cacgagcgtgagctggattctggacgccgacatcagcaggttctttgatacggtcgaccacgattggctgatccgtttcgtgga |
| | | acatcgcataggcgaccagcgtgtgatccgactgatccgcaaatggctgaagggggagcgatggaagacggggtgatcg |
| | | agcccacagacgaggggacgccgcaagggtctgtcatctcgcctctgctggcgaacatctacctgcattacgtcttcgacctc |
| | | tgggcaaaccagtggcgcaagcgccacgccgaggggaacgtcgtaatcgtccgatacgccgacgacgtcgtggttggtttc |
| | | gataaaccacacgatgcgaagcggttcaggcgggccatgcaacaacgactggagcagtttgggctgtcggtacacccgga |
| | | gaaaacccggctgatcgaattcgggcgctttgcggcccggaaccgggcaagccgagggctgggcaaacccgagacgttca |
| | | acttccttggattcacccacatcagtggccgtgcgaaagacggtaggttcatgctcatgcgtaagacgcggagcgaccgtcttc |
| | | gaacggcgctgaagggaatcaaggatgagttgcggcacagatggcaccagtcgattcccgagcaaggggactggttacgc |
| | | cgcgtggttcaagggtacttcaactaccatgcggtgccgaccaacttcgctgcgttgcgagccttccgcgcgcgcgagatcga |
| | | cctctggaggctggcgcttcggcgccgcagccaaaaggacgacaccacgtgggcaaagatgcaccggctcgcgaagcag |
| | | tggatacccaaggctcgcatccttcacccctggccggtcgtgcggtttgacgccaaccacccgaggcaggagcccggtgcg |
| | | cgaatcgcgcacgccgggatctgtgcggggggtgcccagtaatgggcattcctaccgcgacagtgtagcct |
| 3180 | Bacteroides vulgatus | tcagaagttcgtgcgacgtgaagttgcgtaagtgattgtgtaacatcaagattacacctccagttccgtctggagtagcctacgg |
| | | acacgtgcattgggagtaatccttttgtacggagctgtcccgacaaggtagcctttccaaaaggagaagattgaaccgtgagg |
| | | ggaaatcaacggctgataacaccattcagcacgaggagcttggcttagcggtatggtcaacgaaagtgaactgtccgataaac |
| | | atcgtaaggtataacgagccaaaggctgttgacaggctcggaccaaaaggtaagcagtcggctgtcctcttgaactttggggg |
| | | cacacggacattagcactgccggtggaatagacaggacctaacccgcttatttatcacttatgtaaaacacggtaagcccgtat |
| | | ctctccagttcatcagagctggtaagcaagccgcaaggcaagcctacatggggtgtgggtataggatagcggaaaaagcga |
| | | atgcctccctgtaatggggaggatacggattgagccaatatcacgggttggtaaaggtaacatcatccgacacgaaagtgggc |
| | | agacttccgcctatggtgacactttacaagatagtttacagaacttttaaagaaaggaaagcaaatgaacgcaaagaaattggc |
| | | gtgtgcgcccgaagacatagacattcacgaactctggtcgaaaatcgactgggacaaatgtgagcgttttgtccaaaagctgc |
| | | aagcacgtattgtaaaggctcaaagggaaggcagaaataacaaggtgaaagccctgcaatggatgcttacccactcgttctac |
| | | gccaaagcattggcggtaaaacgagttacgactaacaaaggaaaatctacttccggcgttgacaagattacatggtcttctccat |
| | | tggcgaaagccaaagccatattcactctgaaaagacatggctacaagccacaacctttaaaacgtgtaaatatcaaaaagaag |
| | | aacgggaaattacgcccactcggaataccaacgatgaaagaccgtgcgatgcaggcattgtacctaatggcacttgacccga |
| | | tagcggaaaccactggcgacagccattcctacggattccgcagacatcgctgtacacatgatgccatcgagcaatgctacatc |
| | | gttctttctcgttcggtcgctcccgaatggattcttgagggggacatcaaaggatgcttcgaccatatcagccatgcgtggttaat |
| | | caacaatatccctatggataaggagatactacgcaaatggctcgaatgcggatatgtgttcaatggagaactctttcctacggaa |
| | | gagggaaccccacaaggtggcataatatccccaactcttgcaaacatggcacttgacggactgcaagatttgttggaaaagag |
| | | tgtcaagaaatatcaggtgaactacaaaaagatagttcctaagatacatcttgtacgatatgcagatgatttcattgtaacggcaa |
| | | aggataaggaaacgatagaacaggttatacttccattagtacgtaagttccttgcggaaagaggtctgacactatcagaagaaa |
| | | agacaaaaatcacacatataaatgagggatttgactttctcggtttcaatatccgaaagttccgcaataatacgctgctgacaact |
| | | ccatccaaagatgcgcaaaagaggttttgcgaaaaaatccgcaagacgatagaagccaacaagtgtgtaaaacagaagtcac |
| | | ttattatgatgctgaatccaatcataaaaggttggggtaattattataaatatggtacttctgcgaatgtattccaccgaatggattgg |
| | | gaaatcttcaagaaaatatggcaatgggcaagacgcagacatccgcagaaatgtaaaggatgggtaaaggataagtatttcag |
| | | aacactaaatgggcactcgtggagatttgcggcagatatgggcaagaaagataagattgattatctcgaactgacctatctacc |
| | | aaccatccaccatgagaaattcgtcaaggttcggcattatgccaatccgtatgacccatcagataaatcgtactacgaatggag |
| | | agaaacctaccgaatgaagcaaacgctgaaaggcaggcaatctctaataaacatttggaaacgacagaataaagtatgtcctg |
| | | tatgtggagagcgtattgacagggaaagaccttggagtatcactgaacaaatagtcagtggacgaaaggtaagaactcttgta |
| | | cataccagttgtaaacgtaaaatgcaaagtagattatgaatatgaaagtcgaaaatgagatggctctaccgataacaaagtcggt |
| | | aacggtcatgttgcttgagccgtatgcggggaaactcgcacgtacggttcttagaggggaaagctcccgtaagggggctgac |
| | | ctactcgacagttcgggcg |
| 3181 | Chlorobium phaeobacteroides | tcaatttagggtgcgccgggagaccggtaagaggtaaagggtgcaagtccctgcagtgaaaggattagcgatccgcactgg |
| | | ccccgagtcatggcgcataaccgcgaggatatgcgcgaagcattgacaggggtgcatataggccagccattgagcggcg |
| | | aaaatgagcaaatttggggagcccatgccctcctaaaagcagaaggcaatacagacgaagtgcgataacgcgagcacaag |
| | | actgtccccacgacgtcagagaacctggcatgtatgtaagcctcttacacgggaaccgggagatctccgataccacccatcgt |
| | | cgttggcgggtcgcatcgggaaaacgtaatagttgaagccgatgatgtacggtacggagaagtcagacctgcccatactacc |
| | | ggagaagcgagcgaacaaagcgggtaagcccgtagcggagttcgtggagggaaggggtgggaacaagagaaatgcgga |
| | | actgcaaagcatggcccggacacagggtcgggaagccgtgttccaagcgcagggtcgcatacgtgaagcagtaaccagaa |
| | | atcgaggagaaaagctgacagcgcttctgcatcacatctccgtagactgcctgcgctggtcgtacttcgaactgaagaagacc |
| | | gcagcaacgggagtcgatgggataacgtggaaagattacggggaaggactggaagagaaccttgcagacctgcaccggc |
| | | gaatccacacgggagcgtaccgggcacagccatcgcgccggaagtacataccgaaggcaaatggccaacaacgaccgct |
| | | cggcatcgccgcattggaagacaaaattgtacagcgggcagtggtggcgattctcacgccaatctacgaagcggagtttctg |
| | | gggttcagctatggattccgtccgggacgcagtcagcatgatgcgctggatgcactcgcatacgggatcaaggtgaagaaaa |
| | | tcggctgggtattggacgccgatatttcccggttttttgatacgatcagccatgagtggatgatacgctttttggaacaccgtatcg |
| | | gtgacaagcggattgtccgactgattatcaagtggctgaaagctggggtactggaagatagcgtacggatcgaggcagaaga |
| | | aggaacaccgcagggagccgtgatatcaccgctccttgcgaacatctaccttcactatgcttatgacctgtgggctaagcagtg |
| | | gcgggaaaagcactgcaaaggtgatatgatcgtcgtgcgtttcgctgacgatagtgtcgcaggcttccagaacaaggaagat |
| | | ggcgaaagattcctagctgatctgaaagagcgactggcaaagttcgcgctaacgttgcatccagagaaaacccgactgattga |
| | | gttcggcaggtatgctgccaagaaccgacaaagacggggacaagggcggccggaaacctttgactttctcggatttacccac |
| | | atctgtggagagaaagtagtggggaaaggctttcagctcttgcgcaagacgaagcgcggatcagtgagaaccaagctcgga |
| | | gaggtaaagaaagagctgagacggcgaatgcatgtctcagtatcggaacagggaaagtggctgaacagcgtgttgagaggt |
| | | cactatgcctatttcgcagtgcccacaaacacgagggcgttatcggcattccgctaccatgttgccagacgttggatgaagagc |
| | | ctcaggcgccgaagccagcggcatgtgatgacatgggagcgaatgatgatatatattgatcagtacctcccaaacccaaagat |
| | | ccttcacccatggccggaacaacggttttgcgtcaaacactcaagttaggagccggatgcgggaattccgcttgtccggatctg |
| | | tccggggggtgaggggtaactcttgtccctaccgggattttaaatatg |
| 3182 | Coprococcus catus | aatcggaattgtgcgcccagataagggcgagtagtctagcaggtggaatgcctgtctggtaaggtctaaccaaccgccagta |
| | | gcgagtcttgggtgactgtcagtaacggtagttgctaagcgtagacagcgagaaaatagagagtgtgattgagcctcgaaattt |
| | | tgacatttagaaggctgacgttctaatttcgacggaaagcaatacaaatcaattcgcattggtgagaaatggtttgcttctgcggg |
| | | gtcaaagagcactttatgttttacattgtgattattcagtcaactggggagagcctattgtttcttgccattgcaagtatggcaaaca |
| | | actgcaaaacaggagaatgtcaaaagaacagtaggcagtcggatagcttcatagtactgaagaagttgggtaatgccaacgg |
| | | aggaaagggagctacataataatggtctttctgaggacacatcaaccgtactcagggacggaggtattgatggaaacgaaatt |
| | | agaaagaatagcagaaatatcggcaaattcgccgagaccagagttcacatcgttgtatcatctaatcaataaagaaatgcttttg |
| | | caatgtcacaaagaattagacggaaacaaagcagtcggtgttgatgagattactaagaaagagtatgagagaaatctggagca |
| | | aaacatagatgacttggtagaaagactgaaaagaaaatcatacaaaccacagccatcaattagggtatatatccctaaaagcaa |
| | | tggaaaacttcgaccattaggaattgcgtgctatgaggacaaaatagtccaattagcattaaagaagatattggaagctatctatg |
| | | aaccgagatttctgaactgcatgtacggattcagaccaaaccgaggatgtcataacgcaataaaagaactgtataagagattaa |
| | | acaacacgaaaatctgctatattgtggatgctgacatcaaaggattcttcgaccatatgaagcatgaatggattatcaagtttctca |
| | | aactttatattaaagaccccaatataattggtcttgtcaagaagtatcttaaagtgggagtgatggataatggtgaacttatggtga |
| | | atgaagaaggttcagctcaaggcaatatcataagtccaatcctagcaaacatatatatgcacaatgtattgacactatggtataag |
| | | tttattattaccaaagaatgcaagggagataactttctaattgcatatgcagatgattttgttgcaggatttcaatgcaaatgggaag |
| | | cagagaattactacaagcttcttaaagaacgaatggaaaagttcggtttacaattagaagatagtaaaagtcgtctcttacaaagc |
| | | ggtgcttatattgcaagggcaaagcaaaagagcggtgagtgtatcagattacaaacatttgattttctgggattcacattctattgt |
| | | gggcgttcacgcaaaggcatgccatatataatgccaaagacaagctctaagaaattcagacagaaaattcgaggtatcaaggt |
| | | atggttatatgcgaatagagaccaaccgctaaagaaactaatgggtatgctaaatctgaaactaataggtcattataggtactatg |
| | | gcataagttttaatggcagaatgatttcaaactacaaacaacaagtgagagaacttctgtttaaggtgctgaatagacgaagtga |
| | | cagaaagagttatacaagagaaggattcattgaaatgctgaaatattatccattggcaatgccaaagatttatgtcagcttgttttg |
| | | agaatgtaaatattattatgaagagccgtatgcgggaaagccgcacgtacggttctgtgaggggcttacattgtgaggtgtaagt |
| | | ctactcgactgaggtgatg |
| 3183 | Cupriavidus taiwanensis | ttttctttttgtgcgcccggcagggcgcactcacttgagggtgaaagtcccttacacacccggcaaggggaagtgttagccata |
| | | ggcaagggttccccgggcgactgggggtctgaaggaagcccgaggcaaagcgctggcctgacgaacaggaagcggatat |
| | | gaggcgacgcatctgggtaaggcagcaaaaagggccaaagcccagtacttgcacggaacggtgcggcgtagatccgaca |
| | | ggcataagcgtgaaggtgggtgcgtcatacccggggagatctgtaccgctgccacggtgctaccggcgtcgagaggtgtcg |
| | | ggatggcggtgcagaagtcagcagaggccatattaggcgagccgttggcgctgaagggccgaacatgaatgagcgcgatt |
| | | aggacgacagacctcgatgcttaccgacgaagcccaaatgcacgaacgagtgcagcccacagcggaggaaggcgggcg |
| | | gaacctgcccggggccgatgggggtgcggaggctggcacggcggctgttgggcaaacgaaagcgcgggcgccatcgct |
| | | gatggaggcggtggtcgagagaagcaacatgtggctggcgtaccggagggtggtcggcaatggcggtgcggccggggtg |
| | | gatgccttggaggtgacggcgttgcgtgactggctgaaggtgagctggccaagcgtcagggcggcgttgctgggtggtcag |
| | | tacatcccgcagtcggtgcgcgcggtggacattcccaagccttcgggcggggtgaggacactgggcatcccgacggtggtg |
| | | gaccggctgatccagcaggcgctgctgcaggttctccaaccgctctacgaaccggggttctccgagtcgagctatggcttcag |
| | | gccgaggcgcagcgcccagcaggcggtcttgcaggcacagcggtacgtgcaggaaggccggcgatgggtggtggacat |
| | | cgaccttgagaagttcttcgaccgggtcaaccacgacatcctgatgtcgcgggtggcacggcaagtgaaggatgtccgggtg |
| | | ctcaagctgatccggcggtatctggaagcggggctaatgcgtggcggggtggtcgaggcgaggaggcagggcacgccgc |
| | | agggcgggccgctgtcgccgctgctgtcgaacattctgctgacggattgggaccgtgaactcgagaagcgggggttggcgtt |
| | | ctgtcgctacgcggacgactgcaatatctacgtccgaagccaggcagcaggacagaggctgttggccgggatgatgacgttc |
| | | cttgcggagcggctgaatctgcaggtcaatgaggccaagagtgcctgtgcgcggccttgggcacgcaagttcctgggctaca |
| | | gcctgacagcgcatcgtcaggccaagctgcgtattgccccggaaagtctgcaaagactgaccggacgaatcaaggaactga |
| | | tgcgcaaagggcgaggaaggagcctggctcacacgatagcggtgctgaatccggtgctgcgcgggtggatcgggtacttcc |
| | | agcacacccagagcaagcggccattggaggagctggacgggtggatacggcgccggctgcggtgcttgctatggcggca |
| | | ggccaagagccgtgctacccgcactgtgatgctgcgtcggcaaggcttaacggaggatcgcgcgtggcactcggctcgcaa |
| | | cgggcagggcccatggtggaatgccggggctcgccacatgggcgcggccttcccgaagggctacttcgacgccttggggtt |
| | | gatctcgctgctggatactcagcagcgcttgcagtctcgttcgtgaaccgccgtatgcggaaccgcacgtacggtggtgtgag |
| | | agggctgagggggtgacccctcaccctactcaatcctgccgcac |
| 3184 | Chlorobium luteolum | atgacatggagtgcgacgtgaagtcgcgtatttgaactgttctttgcagaaccagttgtgccgtcaactgcccccaccggcaca |
| | | tgcaggatgagcaatcgccctgtgtgaagctaccggaaacgtacccgcgggaaaccgtaggccgacccgtgaggagaagc |
| | | tgaatctgccagtagcaggcggagaggggcaagggggaggatggtatggttaacgcaggtgaatcgtcacaaccatcgttat |
| | | aataagcaagcgaaagctcactgacaacgcttggaccaaaaggtacgcgggcaggtatgctcccttccatgtggagcacaca |
| | | cggccaacaaatcccgccggtggagagacggaacctaacccatccgcatgttctatacgcggaacacggaaaccccgtact |
| | | tccgcccggccatgccgggcaatccgactgtaaggaaggaccatgaaagtgcgggtatgagagtggggaagaagcgaac |
| | | gccgtcctgtaatcgggcggataggggttgcaacatcaccccacgtgaaaacgggctgacgtcctcatggtctcgatttgcaa |
| | | gataacttgaagaacctcccatgcagaaggaacgcaaatgaacacggataaccctgtgtgtgcaccttccggcctcgactgg |
| | | caagggatcaactggtcccggatcaagcgacaggtcaggaggcttcaggcacgtattgcaaaggcaacaaaggaaggccg |
| | | tcatggcagggtgaaagccttgcagtggctgctgacccactcgcacagcggcaaggttcttgccgtcaaacgggtgacggaa |
| | | aaccgggggaaaaacaccccgggagtcgacggagacgtctggaaaacatcaaaagcaaaggccaatgccgcagcatcgc |
| | | tgagacgaagaggctacaagccccttccccttcgaaggacctacattccgaagaagaacggtaagcaacgacctctcggcat |
| | | cccaaccatgaaagacagggccatgcaagcgctctactggcttgcactggagcctgtagcggaaaccaccgcggacggca |
| | | actcgtacgggttccgaccatggcggtcaacagccgacgtggcagaacagtgtttcatctgcctcgcaagacgcgattccgc |
| | | acaatggatacttgaggccgatattgccgggtgcttcgatgctatcagccaccaatggctggtcgacaacatccccatggatac |
| | | gccgatcctccgcaaatggctgaaggcaggcttcgtgttcaacaacgagctctttcccacagcctcgggcacgccgcaaggc |
| | | ggcatcatcagtccgggactggcaaacatgagccttgacggcctcgagcaagcgcttgcaacagcatttccgcaagcgcga |
| | | agacgaggactgaaaatgcatatggtgcgctacgctgacgacttcatcatcactggaaactcgaaagagtggctggaacatg |
| | | agatcatgccggtagtggtcgacttccttaaaaaacgagggctctggctctcagaggaaaagaccagggtaacgcacattac |
| | | ggaaggatttgatttcctcggctggaacatgcgcaagtacgacggcaagctacttatcaagccctcgaaagcgaacatcaagg |
| | | cccacctcaccaaggtgcggggaatcatcaaggcgaaaaagacgatcaagcaggtggatctcatcggtctcctcaaccccgt |
| | | cctgcggggatgggcgaactaccaccggcacagtgtcgccaaagacgtcttcgctcgcaacgaccacgaggtctggtcaat |
| | | gctctggaaatgggcgaaacgacgacacccaaataaaggccttcgatggatcatggacaaatactttcatgccagaggaggc |
| | | aggaaatgggtgttcgtcgccgaagaggcggaccgaaagaaagaacgacggctctttctggaagccagcatgccaatccag |
| | | cgacacgtcaaaatcagaacgaaggcaaacccgcacgatccagtgtggagggagtacttcagcgcccgtagaacacagat |
| | | gtggaaagcgacaccattgagttgccgggtgccttacggcgccttattcgaggcttgagccgtgtgcggtgaaagtcgcacg |
| | | cacggttcttaggggacggtggcgcagtaatgcgccgctgttacccgattatggaaaac |
| 3185 | Chlorobium phaeobacteroides | caacccgttcgtgcgacgtgaaagtcgcatagttgaactgttctttgaaccagttgtgccgtcaactgctcccaccggcacatgc |
| | | aggatgagtaatcgccctgtgtgaagctgccggaaacgtacccgcaggaaaccgtaggctgctccgcgaggaaatgccgaa |
| | | tctgccagtagcaggcggagaggggcaagggagagagtggtcttggctaaggtatctgaatcgctgcaaccgtcgttatgtc |
| | | gaacgagtgaaagctactgtaacactcgaaccaaaaaggtatgcgagtaggtaaacgcctggatggagcgtttacgcggcca |
| | | attactctcgccggggctaggcggagcctacaccacttgagagttcactatgcggaacacggtaagcccgtagtgtcgccc |
| | | agcatactgggcaatcgaaccgtaaggaacgaccatgatactgtgggtaaaggatcgaggagaaagcgaacgcctccctgt |
| | | aatcgggcggataagggctacaatatcgccctatgcgaaagcaggcagacttcctcggggtctcgctttacaagaaaacttga |
| | | agaacctcttatgcagaaggaattgcaaatgaacatgggtagtaccgtgtgtgcaccttccgcctccaactggcaagggatcg |
| | | aatggtctcaggtcaaacggcaggtcaagaggcttcaggcccgtattgtaaaggcaacacaggaaggtcgccactgcaaagt |
| | | aaaagcattgcagtggttactgactcactcgtttagcggcaaagctcttgccgtgaagcgtgtgacggaaaaccggggaaaac |
| | | acaccccgggagttgataaccaaatctggataacgccaaaagcaaaaaccaatgctgtagcatcactaaagcgaaggggtta |
| | | caagcccctccctcttcgaaggatcaatattccgaagaagaacggtaagacccgacctctcggcatcccgacaatgaaagac |
| | | agggccatgcaggcactttatctgcttgcactggaacctgtagcggaaaccactgcggacgataactcgtacgggtttcggcc |
| | | atggcggtcaacagcagacgcgagtgcaagatgttttacctgcctcgctcaacgcaattccgcccaatgggtacttgaggctg |
| | | atattgccagttgcttcgatgctataagccatgagtggttgatcgacaacattccagttgacactgcgatcctccgcctgtggcta |
| | | aaagcaggatttgtgctaaagaacgagctctttcccacagaagccggtacgccgcaaggcggaatcatcagcccggttctgg |
| | | caaacatgtgtcttgacggtctcgagaaggcgctggcgaaagcctttccgcaagcaaagaaacgcggcctgaaaatgcacat |
| | | ggtgcgatacgctgacgactttgttataacaggaaactcgaaagagttgctggaaaacgaggtcttgccagtggtggtcgaatt |
| | | cttggctgaaagagggttattcctctctccggaaaagactaagataacgcacattacggaaggctttgatttcctcggctggaat |
| | | gtacgcaagtacagcggcaaactgcttatcaagccgtcgaaagtgaataccaaggcccacctcctgaaggtgcgggaaatca |
| | | ttaaaggtaataaaacggccaaacaggtaagcttgatctacctcctcaatcccataatgaggggatgggcgaactaccatcaac |
| | | atgtggtcgccaagaaatcctttgctcgcaatgacgcagaaatctggtcaatgctctggaaatgggcaaaaagacgacaccca |
| | | aacaaaggaatgcgatgggtcaaggcacggtacttcaagacccaaaacgcccgcaactgggtgtttgccgctaaggatgag |
| | | gccacaggcaaggaaatcagacttgtcactgaagccgatacaccaatcaagcgacatgtcaaaattaagtcaaaggcgaacc |
| | | ctcacaatccagtgtgggccgaatactttgcagcccgcaacaaacagatgaagaaatcagcaccgatgtacagccgggcgtc |
| | | acaaggcgccttatgcgaggcttgagcccagtgcggtgaaagtcgcacgctgggttcttaggggacggtggcgtagcaatg |
| | | cgccgccgttacccgacatcgagtcgt |
| 3186 | Geobacter uraniireducens | tgtttattcggtacgccaggccaagcctggctgatctatctgactgaaaagtgtcagacatgtaaattgctcgttcaccatgtagtc |
| | | gcggtctgtcacgagagggtaaccaaacgtggggagtaagaccggttctttgtaagaaaagagcgaagggtacgataactac |
| | | cctgacgacgaagacgcgagcccagcgcaagcgaacccgtttcggcctcgttacacgtagttgacagtggccaaccttccg |
| | | aatgtggtgaagcctgccatccaccggtgataaacgagcgaaagaaccggtgggactgtccggggtgattggggagggcg |
| | | cgtgtcgaaagatcagccgaggaacctgggaggtccgacgtaattcccaaagcgaaagggtttcgccgaagggagaacgc |
| | | cgacggggaatcaataacccgccaagcgtgtacggtcggaattcggagcggcccatagtagccgggaagcggggtaatgc |
| | | ccgtggagccaaggggccgtacttcagtcatgtttacacggaaagagaggagacccgcttgagcgacagacgctccataac |
| | | ggaatggatggccgaaggattcgaacctgaacccggactgccggtaaaagtctcccttctgcggtggaaactgagcagcaa |
| | | ggccaaacgagaaccgcagttccggttctatgccctttacgaccgaatccatcgtcgtgatgtacttgaaacggcgtggttgcg |
| | | agtaaaggcgaacaagggcgcgccgggagtcgatggtgtcagcattgaatccatcgaggtgcgggcagacggaatatccg |
| | | gctatcttgacgaaattcaagaaagcctgagaaccaaaaactacaaaccatccccggtgcgtcgcgtctacatcaccaagccc |
| | | aacggcaaactgcgcccgctcggcataccgtgcgtgcgggacagaattgtacaggcagccgtactccttatcctcgaaccga |
| | | tatttgaggtggactttctcgactgctcccatggatttcgtcccaaaaggcgtccccatggagccctcgaccaagtcggtaataa |
| | | cctccaactcggacggcaggaagtgtatgacgcggatctttcgagctactttgacagcatcccccacgagcacctgatagtag |
| | | aacttgagcggcgcatagccgaccggagtgtcctcaaactgatccggcaatggcttcacagtccggtcagagaagaagacg |
| | | gcagcatcagccgtcccaaacaagggacgccacaaggggggtaatatctccccttcttgccaacatctaccttcaccggctc |
| | | gaccgggcctttcacgaagaagcagacagcccctaccacttcgccagagcgcgcatggtacggtttgccgacgacttcgtag |
| | | taatggccagacacatgggcaatcgcataaccggatggctggaagagaaactcgaaacagaccttggactcagcatcaacc |
| | | gtgacaaaaccggtattgtcagaatgaacaagaaagagagcctgaactttctgggattcaccctccgctatgaccgtgacctgc |
| | | ggggcagagattgggattatctcaacatcatgccgagtaagaaggccataggagcactcaagggaaaaatccgggagaaga |
| | | cccgaagcggatacaaaaagccgcttgtcgaagcaattgccgaggtaaacagcatccttcgcggatggggcaactacttcga |
| | | ctacgggtatccccggaaagtatttcgagacgtgaatcattacacgaggactcgctttcgaatatttctgaacaatcggagccaa |
| | | aggcggagcaaacccttcagggcaggtgaaagcctgtatgccggacttaagcgatatggtctggtttacctgtaacagcaaag |
| | | ccacaactcctcaataatgccgtgcagcaagaggctgataggtaagccgtatgcgggaaatccgcacgtacggtttgacgag |
| | | ggggagctggttaaggcaactatggtaaggctacttaggcaccgccaaacgaaaggggcggaaacggacaagccaaacct |
| | | aatgatgtcgtaacctgcttcctactctactggttaattg |
| 3187 | Geobacillus sp | ctcttttcatgtgcgcccggcatgggtgtagtctatagggtgaaagtcccgaactgcgaaggcagaagtagcagttagcttaac |
| | | gcaagggtgtctgcggcgacgcagaatctgaaggaagcgggcggcaaacttccggtctgaggaacacgaacttcatagga |
| | | ggctgggtatcattgggtgagtttgcatgacaaaacgaagccctttctgccgaaggtgataccgagtaaatgaagcagatagat |
| | | ggaaggaaagactgcactcttacccggggaggtctgtccgggaagccaagtgcgcttggcaaccgttggagcgatccaacg |
| | | ctgaacggacagaagtcagcagaggtcatagtactcgtctagcttaagatagaaggggaaggaccgaaccatgaaggagaa |
| | | cggccactaggcgttcatcttctttgatgaaacagacaacccgaaagggcctgcttgagggaggaaatggtgaagtccatgg |
| | | gggacctcaagagggtggagaagaagatggcacgaatagaacggatcgttcacgtagagaggaagaattggaatgtggat |
| | | gaaacagatactgtcacgggagaatctcctgcgagcactcaaacaagtggaaaagaataaagggtcccatggaaccgatgg |
| | | aatgtccgtcaaagacctgcgaagacacctcgtggaacattgggacgtgatacggcgtgctttggaagaagggacctacgaa |
| | | ccttgcccggtccgacgggtcgaaatcccgaaaccgaacggaggagtcaggttactaggaatcccgaccgtgacagaccg |
| | | gttcatccaacaggccatcgcccaagtgctcacgccgatctttgacccatccttttcggaacacagctacgggtttcgtcccggt |
| | | cgaagaggacacgacgcggtgaaaaaggcgaagcagtatattcaggaaggatatacatgggtggtagatatcgacttggaa |
| | | aagttctttgatcgagtcaaccatgacaaactgatggggatattagcgaaacgaattccagacaaaatcctcctaaagttgatac |
| | | ggaagtatttacaggcaggggtcatgatcaacggggggtcatggaaacacaagaggggactccacaaggagggccgctc |
| | | agtccacttttgtccaacattctcttggatgagctggacaaagaattggaaaaacgagggcacaaatttgtacggtatgcggatg |
| | | actgcaatatctacgtaaggacgaagaaggccggggaacgggtgatgaaatcgatcacggcattcattgaaaagaaactccg |
| | | gctgaaagtcaacgaaaccaaatcggcagtggatcgaccgtggaggagaaaattcctcggttttagcttcaccccaaataagg |
| | | agccaaaaatccggatcgcaaaggaaagtattcggcgcatgaagcaaaggatgcgcaccatgacgagtcgatcgaaaccg |
| | | attcccatgctcgagcgaatcgaacagctcaaccagtacattctgggatggtgtggatacttctcgttagcagagactccaagtg |
| | | tgttcaaagaactagatggatggattcaacgaaggctgcgcatgtgccaatggaaagagtggaaacttccgagaaccagagt |
| | | ccgaaaactgcaaagtttaggagtgcccaagcggaaagcatatgaatggggaaacactcggaagaaatattggagagtggc |
| | | cgctagtcccatcttgcataaagcccttggcaactcctattgggagagccaagggctgaagagtctttatcaacgatatgaatct |
| | | ctgcgtcagacttaatggaaccgccgtataccgaacggtacgtacggtggtgtgagaggacgagggttaatcaccctctccta |
| | | ctcgatcattctgttg |

In some embodiments, the 3′ or 5′ intron segments and/or 3′ or 5′ exon segments are derived from a gene selected from a species selected from: Cyanobacterium Anabaena sp., T4 phage, Coxiella burmetii, Azoarcus, Tetrahymena thermophila, and Staphylococcus phage Twort. In some embodiments, the 3′ or 5′ intron segment and/or 3′ or 5′ exon segment are developed from permuting at a position along a Cyanobacterium Anabaena sp., T4 phage, Coxiella burnetii, Azoarcus, Tetrahymena thermophila, and Staphylococcus phage Twort intron and/or exon sequence. In some embodiments, the 5′ or 3 monotron element are derived from a gene selected from a species selected from: Cyanobacterium Anabaena sp., T4 phage, Coxiella burnetii, Azoarcus, Tetrahymena thermophila, and Staphylococcus phage Twort. In some embodiments, the 3′ or 5′ intron segment and/or 3′ or 5′ exon segment are developed from permuting at a position along a Cyanobacterium Anabaena sp., T4 phage, Coxiella burnetii, Azoarcus, Tetrahymena thermophila, and Staphylococcus phage Twort intron and/or exon sequence.

In some embodiments, the intron segments and/or exon segments of a provided polynucleotide are derived from a gene from the same species (e.g., a polynucleotide comprises Azoarcus 3′ and 5′ exon segments and Azoarcus 3′ and 5′ intron segments). In other embodiments, the 3′ or 5′ intron segments or 3′ or 5′ exon segments of a provided polynucleotide are derived from genes of different species (e.g., a polynucleotide comprises an Anabaena intron segment and Staphylococcus phage Twort exon segment). In certain embodiments, the monotron element of a provided polynucleotide is derived from a gene of a different species than the 3′ or 5′ intron segments and/or 3′ or 5′ exon segments (e.g., a polynucleotide comprises a Staphylococcus phage Twort montron element and an Anabaena intron segment). In some embodiments, use of genes of one species of an intron segment and/or exon segment may allow for more efficient or effective circularization or self-splicing of one or more polynucleotides as compared to another gene of a different species. In certain embodiments, the gene used of one species develop an intron segment may more efficiently promote the interaction between an intron segment and a nucleophile (e.g., form a more efficient or effective binding pocket that promotes the transesterification reaction of a splice site nucleotide) as compared to an intron segment developed from a gene of a different species. In some embodiments, the gene of one species from which an intron segment is derived may be more efficient in forming a binding pocket for a nucleophile as compared to a different gene of the same species. In some embodiments, the species of gene from which the intron segment is derived may be more efficient in forming a binding pocket for a nucleophile as compared to a species of genes comprising the same and/or homologous sequence from a different species.

As described herein, in some embodiments, a provided polynucleotide comprises an intron segment and/or exon segment derived from permuting at a position along a Group I or Group II gene selected from Table A or Table B. Location or position of the permutation sites may enhance the ability of an intron segment to effectively splice and/or circularize in a provided polynucleotide. In some embodiments, the Group I or Group II genes are permuted at a position that enhances splicing or circularization activity of an intron segment of a provided polynucleotide as compared to a different permutation site. In certain embodiments, the Group I or II genes are permuted at a position in an intron segment of a provided polynucleotide that enhances the provided polynucleotide's ability to self-circularize as compared to a different permutation site. In some embodiments, the Group I or II genes are permuted at a position that enhances or promotes the splicing activity of an intron segment to another intron segment, monotron element and/or exon segment. In some embodiments, the Group I or II genes are permuted at a position that allows the intron segment to more efficiently splice or self-splice than an intron segment permuted at a different position. In certain embodiments, a position of a permutation site may promote the interaction between an intron segment and a nucleophile (e.g., form a more efficient or effective binding pocket that promotes the transesterification reaction of a splice site nucleotide).

As described herein, permutation sites positions are described to mean that the permutation of the natural or synthetic intron occurs at the junction between the listed amino acid and the adjacent downstream amino acid (e.g., an Anabaena position 189 permutation site corresponds herein to a permutation site between amino acids 189 and 190).

In some embodiments, a provided polynucleotide comprises an intron segment derived from permuting at a position along a gene selected from a species selected from: Cyanobacterium Anabaena sp., T4 phage, Coxiella burnetii, Azoarcus, Tetrahymena thermophila, and Staphylococcus phage Twort. In certain embodiments, a provided polynucleotide comprises an intron segment derived from permuting a Cyanobacterium Anabaena sp. gene. In these embodiments, the permutation site of the Cyanobacterium Anabaena sp. gene may be downstream relative to amino acid positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, or 265 of the Cyanobacterium Anabaena sp. gene. In certain embodiments, a provided polynucleotide comprises an intron segment derived from permuting an Azoarcus gene. In these embodiments, the permutation site of the Azoarcus gene may be downstream relative to amino acid positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, or 221 of the Azoarcus gene. In certain embodiments, a provided polynucleotide comprises an intron segment derived from permuting an Coxiella burnetii gene. In these embodiments, the permutation site of the Coxiella burnetii gene may be downstream relative to amino acid positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, or 390 of the Coxiella burnetii gene. In certain embodiments, a provided polynucleotide comprises an intron segment derived from permuting a Tetrahymena thermophila gene. In these embodiments, the permutation site of the Tetrahymena thermophila gene may be downstream relative to amino acid positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, or 436 of a Tetrahymena thermophila gene. In certain embodiments, a provided polynucleotide comprises an intron segment derived from permuting an T4 phage (td) gene. In these embodiments, the permutation site of the T4 phage (td) gene may be downstream relative to amino acid positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, or 289 of the T4 phage (td) gene. In certain embodiments, a provided polynucleotide comprises an intron segment derived from permuting a Staphylococcus phage Twort gene. In these embodiments, the permutation site of the Staphylococcus phage Twort gene may be downstream relative to amino acid positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277,278, 279, 280, or 281 of the Staphylococcus phage Twort gene.

Also provided herein are methods of identifying an exon and/or intron element or identifying a combined accessory element comprising a mutated Group I or Group II exon and/or intron segment (as described herein) that allows production of a circular RNA that is translatable or biologically active inside a eukaryotic cell. In some embodiments, such a method comprises:
(i) inserting 5′ and 3′ Group I or Group II intronic sequences derived from a database of native intronic sequence into a precursor RNA polynucleotide;
(ii) transcribing the polynucleotide into RNA in vitro or allowing the polynucleotide to be transcribed into RNA by a cell; and
(iii) determining the circularization efficiency of the RNA produced by the polynucleotide by identifying the amount of circularized RNA, the amount of excised intronic sequences, the amount of precursor RNA remaining after circularization, and combinations thereof.

In some embodiments, the mutated Group I or Group II exon and/or intron element or segment comprises a deletion, insertion or substitution of at least one nucleotide, including but not limited to a nucleotide substitution of one or both the dinucleotides of the 5′ and/or 3′ Group I splice site dinucleotides. In some embodiments, the 5′ or 3′ Group I or Group II intronic sequences, or combinations thereof are sequenced. In some embodiments, the method further comprises comparing the circularization efficiency of the polynucleotide with a polynucleotide comprising a native intronic sequence, or a parent polynucleotide.

Also provided herein are methods of identifying or determining a polynucleotide sequence that improves RNA circularization efficiency compared to a polynucleotide comprising a native intronic sequence or to a parent polynucleotide with a known sequence, the method comprising modifying a DNA sequence encoding the precursor RNA polynucleotide described herein comprising:
(i) modifying at least one nucleotide and/or altering the length of the 5′ intron element and/or 3′ intron element of the DNA sequence encoding the precursor RNA polynucleotide described herein;
(ii) altering the length of the 5′ and/or 3′ internal and/or external spacer sequence of the DNA sequence encoding precursor RNA polynucleotide;
(iii) altering the length of the 5′ and/or 3′ internal duplex sequence of the DNA sequence encoding the precursor RNA polynucleotide;
(iv) altering the length of the 5′ and/or 3′ exon sequence of the DNA sequence encoding the precursor RNA polynucleotide; or
(v) combinations thereof, and
transcribing the polynucleotide comprising the DNA sequence into RNA in vitro or allowing the polynucleotide comprising the DNA sequence to be transcribed into RNA by a cell; and determining the circularization efficiency of the RNA produced by the polynucleotide comprising the DNA sequence by identifying the amount of circularized RNA, the amount of excised intronic sequences, the amount of precursor RNA remaining after circularization, and combinations thereof. In some embodiments, the method further comprises comparing the circularization efficiency of the polynucleotide with a polynucleotide comprising a native intronic sequence, or a parent polynucleotide.
   d. Spacer

In various embodiments, a provided polynucleotide (e.g., a DNA template, a precursor RNA polynucleotide, or a circular RNA polynucleotide) comprises one or more spacers.

In certain embodiments, the DNA template, precursor linear RNA polynucleotide and circular RNA provided herein comprise a 5′ and/or a 3′ spacer. In some embodiments, the polynucleotide comprises one or more spacers in the intron elements. In some embodiments, the polynucleotide comprises one or more spacers in the exon elements. In some embodiments, the polynucleotide comprises a spacer in the 3′ intron fragment (also referred to as “5′ external spacer”). In some embodiments, the polynucleotide comprises a spacer in the 5′ intron fragment (also referred to as “3′ external spacer”). In some embodiments, the polynucleotide comprises a spacer in the 3′ exon fragment (also referred to as “5′ internal spacer”). In some embodiments, the polynucleotide comprises a spacer in the 5′ exon fragment (also referred to as “3′ internal spacer”).

In certain embodiments, the polynucleotide comprises a spacer in the 3′ intron fragment and/or a spacer in the 5′ intron fragment. In some embodiments, the 5′ external spacer is located 5′ to the 3′ permuted intron segment. In some embodiments, the 5′ internal spacer is located 3′ to the 3′ exon segment. In some embodiments, the 3′ external spacer is located 3′ to the 5′ permuted intron segment. In some embodiments, the 3′ external spacer is located 5′ to the 5′ exon segment.

In certain embodiments, the polynucleotide comprises a 5′ external spacer located between a leading untranslated sequence and the 5′ or 3′ intron element. In certain embodiments, the polynucleotide comprises a 3′ external spacer located between the 3′ or 5′ intron element and a lagging untranslated sequence.

In certain embodiments where the polynucleotide comprises a monotron, the polynucleotide can comprise an internal spacer sequence positioned between the terminal element and the intervening region, and/or between the intervening region and the monotron element. In certain embodiments, the polynucleotide can comprise an external spacer positioned adjacent to the terminal element and/or an external spacer positioned adjacent to the monotron element.

In certain embodiments, the spacers aid with circularization or protein expression due to symmetry created in the overall sequence of the precursor RNA polynucleotide. In certain embodiments, including a 5′ internal spacer and/or including a spacer between the 3′ group I intron fragment and the intervening region may conserve secondary structures in those regions by preventing them from interacting, thus increasing splicing efficiency. In certain embodiments, there is a spacer, for example, between the 3′ permuted intron segment and the intervening region, wherein the spacer may prevent structured regions of an IRES or aptamer of a TIE comprised in the intervening region from interfering with the folding of the 3′ permuted intron segment or reduces the extent to which this occurs.

In some embodiments, the polynucleotide further comprises an aptamer. In some embodiments, the aptamer is synthetic.

In some embodiments, the first spacer (e.g., between the 3′ group I or II intron fragment and intervening region) and second spacer (e.g., between the two expression sequences and intervening region) comprise additional base pairing regions that are predicted to base pair with each other and not to the first and second duplex regions. In other embodiments, the first spacer (e.g., between 3′ group I or II intron fragment and intervening region) and second spacer (e.g., between the one of the intervening region and 5′ group I or II intron fragment) comprise additional base pairing regions that are predicted to base pair with each other and not to the first and second duplex regions.

In certain embodiments, the polynucleotide comprises a first (5′) and a second (3′) spacer. In some embodiments, the polynucleotide comprises a 5′ external spacer and a 3′ external spacer, wherein the spacers comprise additional base pairing regions that are predicted to base pair with each other and not to the first and second duplex regions. In other embodiments, the polynucleotide comprises a 5′ internal spacer and a 3′ internal spacer, wherein the spacers comprise additional base pairing regions that are predicted to base pair with each other and not to the first and second duplex regions. In some embodiments, such spacer base pairing brings the permuted intron segments in close proximity to each other, which may increase splicing efficiency. Additionally, in some embodiments, the combination of base pairing between the first and second duplex regions, and separately, base pairing between the first and second spacers, promotes the formation of a splicing bubble containing the permuted intron segments flanked by adjacent regions of base pairing.

Typical spacers are contiguous sequences with one or more of the following qualities: (1) predicted to avoid interfering (e.g., forming duplex) with proximal structures, for example, the IRES, expression sequence, aptamer, or intron; (2) is at least 5 nt long and no longer than 100 nt; (3) is located adjacent to the permuted intron segment; and (4) contains one or more of the following: (a) an unstructured region at least 5 nt long, (b) a region of base pairing at least 5 nt long to a distal sequence, such as another spacer, and (c) a structured region at least 5 nt long limited in scope to the sequence of the spacer. In various embodiments, a spacer is not predicted to form a duplex of more than 8 nucleotides in length with any sequences within 250 nucleotides in either direction. In some embodiments, the spacer is not predicted to form a duplex of more than 8 nucleotides in length with any sequences within 1000 nucleotides in either direction. In some embodiments, the spacer comprises an unstructured, structured or randomly generated polynucleotide sequence. Spacers may have several regions, including an unstructured region, a base pairing region, a hairpin/structured region, and combinations thereof. In an embodiment, the spacer has a structured region with high GC content. In an embodiment, a region within a spacer base pairs with another region within the same spacer. In an embodiment, a region within a spacer base pairs with a region within another spacer. In an embodiment, a spacer comprises one or more hairpin structures. In an embodiment, a spacer comprises one or more hairpin structures with a stem of 4 to 12 nucleotides and a loop of 2 to 10 nucleotides.

In some embodiments, a spacer sequence is at least 5, 6, 7, 8, 9, 10, 11, 12, 13, 14, 15, 16, 17, 18, 19, 20, 25, or 30 nucleotides in length. In some embodiments, a spacer sequence is no more than 100, 90, 80, 70, 60, 50, 45, 40, 35, or 30 nucleotides in length. In some embodiments, a spacer sequence is between 5 and 50, 10 and 50, 20 and 50, 20 and 40, and/or 25 and 35 nucleotides in length. In certain embodiments, a spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In some embodiments, the spacer sequence is at least 5 nucleotides in length, and/or about 5 to about 60 nucleotides in length.

In some embodiments, a spacer sequence is a polyA sequence. In some embodiments, a spacer sequence is a polyAC sequence. In some embodiments, a spacer comprises about 10%, 20%, 30%, 40%, 50%, 60%, 70%, 80%, 90%, or 100% polyAC content. In some embodiments, a spacer comprises about 10%, 20%, 30%, 40%, 50%, 60%, 70%, 80%, 90%, or 100% polypyrimidine (C/T or C/U) content.

e. Duplex

In various embodiments, a provided polynucleotide (e.g., a DNA template, a precursor RNA polynucleotide, or a circular RNA polynucleotide) comprises one or more duplexes.

In some embodiments, the polynucleotide comprises a 5′ external duplex located within the 3′ intron fragment. In some embodiments, the polynucleotide comprises a3′ external duplex located within the 5′ intron fragment. In some embodiments, the polynucleotide comprises a 5′ internal duplex sequence and a 3′ internal duplex sequence. In some embodiments, the polynucleotide comprises a 5′ internal duplex located within the 3′ exon fragment. In some embodiments, the 5′ internal duplex sequence is positioned between the 5′ exon element and the intervening region. In some embodiments, the polynucleotide comprises a 3′ internal duplex located within the 5′ exon fragment. In some embodiments, the 3′ internal duplex sequence is positioned between the intervening region and the 3′ exon element. In certain embodiments, the polynucleotide comprises a 5′ external duplex located within the 3′ intron fragment and a 3′ external duplex located within the 5′ intron fragment. In some embodiments, the polynucleotide comprises a 5′ internal duplex located within the 3′ exon fragment and a 3′ internal duplex located within the 5′ exon fragment. In some embodiments, the polynucleotide comprises a 5′ external duplex, 5′ internal duplex, a 3′ internal duplex region, and a 3′ external duplex.

In some embodiments, the polynucleotide comprises a monotron element, intervening region, and terminal element, and a 5′ internal duplex sequence and a 3′ internal duplex sequence. In some embodiments, if the terminal element is upstream of the monotron element, the 5′ internal duplex sequence is positioned between the terminal element and the intervening region, and the 3′ internal duplex sequence is positioned between the intervening region and the monotron element. In some embodiments, if the monotron element is upstream of the terminal element, the 5′ internal duplex sequence is positioned between monotron and the intervening region, and the 3′ internal duplex sequence is positioned between the intervening region and the terminal element. In some embodiments, the 5′ or 3′ internal duplex is positioned adjacent to a 5′ or 3′ internal spacer.

In some embodiments, the polynucleotide comprises a first (5′) duplex and a second (3′) duplex (e.g., a 5′ external duplex region and a 3′ external duplex region). In certain embodiments, the first and second duplex regions may form perfect or imperfect duplexes. Thus, in certain embodiments, at least 75%, 80%, 85%, 90%, 91%, 92%, 93%, 94%, 95%, 96%, 97%, 98%, 99% or 100% of the first duplex and second duplex may be base paired with one another. In some embodiments, the duplexes regions are predicted to have less than 50% (e.g., less than 45%, less than 40%, less than 35%, less than 30%, less than 25%) base pairing with unintended sequences in the RNA (e.g., non-duplex sequences). In some embodiments, the 5′ internal duplex sequence and 3′ internal duplex sequence are at least 80%, at least 85%, at least 90%, at least 91%, at least 92%, at least 93%, at least 94%, at least 95%, at least 96%, at least 97%, at least 98%, at least 99% or 100% complementary. In some embodiments, including such first duplex and second duplex on the 5′ and 3′ ends of the precursor RNA strand, respectively, and adjacent or very close to the permuted intron segment, bring the permuted intron segments in close proximity to each other, increasing splicing efficiency.

In some embodiments, a duplex, whether, e.g., a 5′ internal duplex sequence or 3′ internal duplex sequence, is 3-100 nt in length (e.g., 3-75 nt in length, 3-50 nt in length, 20-50 nt in length, 35-50 nt in length, 5-25 nt in length, 5-20 nt in length, 9-19 nt in length). In some embodiments, a duplex has a length of about 9 to about 50 nt. In one embodiment, a duplex has a length of about 9 to about 19 nt. In one embodiment, a duplex has a length of about 5 to about 20 nt nucleotides in length, inclusive. In one embodiment, the 5′ internal duplex sequence and 3′ internal duplex sequence are each independently about 9 to about 50 nt, about 9 to about 19 nt, or about 5 to about 20 nt nucleotides in length, inclusive. In one embodiment, a duplex has a length of about 20 to about 40 nt. In some embodiments, a duplex is about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nt in length. In certain embodiments, a duplex has a length of about 30 nt. In certain embodiments, the 5′ and 3′ internal duplex sequences are predicted to form a contiguous duplex. In some embodiments, the contiguous duplex has a length of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nt in length. In some embodiments, the contiguous duplex has a length of no longer than 35 nucleotides. In some embodiments, at least one of the exon segments is less than 15 nucleotides in length.

In some embodiments, the 5′ internal duplex sequence and/or 3′ internal duplex sequence each have a GC content of at least 10%.

In other embodiments, the polynucleotide does not comprise of any duplex to optimize translation or circularization.

f. Affinity Sequences

In various embodiments, a provided polynucleotide (e.g., a DNA template, a precursor linear RNA polynucleotide, or a circular RNA polynucleotide) may comprise an affinity sequence (or affinity tag) In some embodiments, a precursor RNA polynucleotide comprises at least one affinity tag. In some embodiments, the affinity tag is located in the 3′ intron element. In some embodiments, the affinity tag is located in the 5′ intron element. In some embodiments, both (3′ and 5′) intron elements each comprise an affinity tag. In some embodiments, the 5′ affinity tag is located 5′ to the 3′ permuted intron segment. In some embodiments, the 3′ affinity tag is located 3′ to the 5′ permuted intron segment.

In some embodiments, the polynucleotide comprises a monotron element comprising an affinity tag and/or terminal element comprising an affinity tag. In some embodiments, the terminal element comprises (a) a 5′ affinity tag if the terminal element is located upstream of the monotron element, wherein the 5′ affinity tag is located 5′ to the terminal element; or (b) a 3′ affinity tag if the monotron element is located upstream of the terminal element, wherein the 3′ affinity tag is located 3′ to the terminal element. In some embodiments, the monotron element comprises (a) a 3′ affinity tag if the terminal element is located upstream of the monotron element, wherein the 3′ affinity tag is located 3′ to the monotron element; or (b) a 5′ affinity tag if the monotron element is located upstream of the terminal element, wherein the 5′ affinity tag is located 5′ to the monotron element. In some embodiments, if the precursor RNA polynucleotide comprises an external spacer, the 5′ or 3′ affinity tag is positioned adjacent to the external spacer.

In one embodiment, an affinity tag of the 3′ intron element is the length as an affinity tag in the 5′ intron element. In some embodiments, an affinity tag of the 3′ intron element is the same sequence as an affinity tag in the 5′ intron element. In some embodiments, the affinity sequence is placed to optimize oligo-dT purification.

In some embodiments, the one or more affinity tags present in a precursor RNA polynucleotide are removed upon circularization. See, for example, FIGS. 97A and 97B from WO2022261490, which are incorporated by reference herein in entirety. In some embodiments, affinity tags are added to remaining linear RNA after circularization of precursor RNA is performed. In some such embodiments, affinity tags are added enzymatically to linear RNA. The presence of one or more affinity tags in linear RNA and their absence from circular RNA can facilitate purification of circular RNA. In some embodiments, such purification is performed using a negative selection or affinity-purification method. In some embodiments, such purification is performed using a binding agent that preferentially or specifically binds to the affinity tag.

In some embodiments, an affinity sequence, such as biotin, is added to linear RNA by ligation. In some embodiments, an oligonucleotide comprising an affinity sequence is ligated to linear RNA. In some embodiments, an oligonucleotide conjugated to an affinity handle is ligated to the linear RNA. In some embodiments, a solution comprising the linear RNA ligated to the affinity sequence or handle and the circular RNA that does not comprise an affinity sequence or handle are contacted with a binding agent comprising a solid support conjugated to an oligonucleotide complementary to the affinity sequence or to a binding partner of the affinity handle, such that the linear RNA binds to the binding agent, and the circular RNA is eluted or separated from the solid support.

In some embodiments, an affinity tag comprises a polyA sequence or is a polyA affinity tag. In some embodiments the polyA sequence is at least 15, 30, or 60 nt in length. In some embodiments, the affinity tag comprising a polyA sequence is present in two places in a precursor linear RNA. In some embodiments, one or both polyA sequences are 15-50 nt in length. In some embodiments, one or both polyA sequences are 20-25 nt in length. In some embodiments, the polyA sequence(s) is removed upon circularization. Thus, an oligonucleotide hybridizing with the polyA sequence, such as a deoxythymidine oligonucleotide (oligo(dT)) conjugated to a solid surface (e.g., a resin), can be used to separate circular RNA from its precursor RNA.

Any purification method for circular RNA described herein may comprise one or more buffer exchange steps. In some embodiments, buffer exchange is performed after in vitro transcription (IVT) and before additional purification steps. In some such embodiments, the IVT reaction solution is buffer exchanged into a buffer comprising Tris. In some embodiments, the IVT reaction solution is buffer exchanged into a buffer comprising greater than 1 mM or greater than 10 mM one or more monovalent salts, such as NaCl or KCl, and optionally comprising EDTA. In some embodiments, buffer exchange is performed after purification of circular RNA is complete. In some embodiments, buffer exchange is performed after IVT and after purification of circular RNA. In some embodiments, the buffer exchange that is performed after purification of circular RNA comprises exchange of the circular RNA into water or storage buffer. In some embodiments, the storage buffer comprises 1 mM sodium citrate, pH 6.5.

g. Leading Sequences & Lagging Sequences

In various embodiments, provided polynucleotide (e.g., a DNA template, a precursor linear RNA polynucleotide, or a circular RNA polynucleotide) comprises a leading untranslated sequence. In some embodiments, the leading untranslated sequence is located at the 5′ end in the 3′ intron fragment (also referred to as “5′ leading sequence”). In some embodiments, the leading untranslated sequence comprises the last nucleotide of a transcription start site (TSS). In some embodiments, the TSS is chosen from a viral, bacterial, or eukaryotic DNA template. In one embodiment, the leading untranslated sequence comprises the last nucleotide of a TSS and 0 to 100 additional nucleotides. In some embodiments, the TSS is a spacer. In some embodiments, the leading untranslated sequence contains a guanosine at the 5′ end.

In various embodiments, provided polynucleotide (e.g., a DNA template, a precursor linear RNA polynucleotide, or a circular RNA polynucleotide) comprises a lagging untranslated sequence (also referred to as “trailing sequence”). In some embodiments, the lagging untranslated sequence is located at the 3′ end. In some embodiments, the polynucleotide comprises a 3′ external spacer located between the 3′ intron element and a lagging untranslated sequence. In some embodiments, the polynucleotide a leading untranslated sequence at the 5′ end. In some embodiments, the polynucleotide comprises a 5′ external spacer located between a leading untranslated sequence and the 5′ intron element.

In some embodiments, the polynucleotide comprises a monotron element and a leading untranslated sequence. In some embodiments, the polynucleotide comprises a 5′ external spacer positioned between a leading untranslated sequence and either the terminal element or monotron element. In some embodiments, the polynucleotide comprises a monotron element and a lagging untranslated sequence. In some embodiments, the polynucleotide comprises a 3′ external spacer positioned between the lagging untranslated sequence and either the monotron element or terminal element.

In some embodiments, the lagging untranslated sequence comprises a restriction site sequence or a fragment thereof. In certain embodiments, the restriction site sequence or fragment thereof is used to linearize the polypeptide (e.g., DNA template). In some embodiments, the restriction site sequence is derived from a natural viral, bacterial or eukaryotic DNA template.

### B. Monotron Element

Provided herein is a precursor RNA polynucleotide comprising a monotron (also called a monotron element or monotron sequence) and a terminal element (also called a terminal sequence). In some embodiments, the monotron has ribozymatic activity that allows it to enzymatically self-cleave. In some embodiments, the monotron is capable of forming a phosphodiester bond with a terminal sequence, i.e., a sequence containing a splice site dinucleotide and optionally a natural exon sequence or fragment thereof. In some embodiments, the precursor RNA polynucleotide comprises a terminal element; an intervening region, and a monotron element.

In some embodiments, the precursor RNA polynucleotide comprises, in the following order, (a) a terminal element; (b) an intervening region, and (c) a monotron element. In some embodiments, the terminal sequence is upstream of the monotron sequence in the precursor RNA polynucleotide. In such embodiments: (i) the terminal element comprises a splice site nucleotide, (ii) the monotron element comprises a splice site dinucleotide at or near the 5′ end of the monotron, and (iii) the monotron element is capable of interacting with a nucleophile that is capable of cleaving at the splice site dinucleotide at or near the 5′ end of the monotron, where the cleavage product of (iii) comprises a 5′ splice site nucleotide that is capable of cleaving at the splice site nucleotide of the terminal element. In some embodiments, the nucleophile is a free nucleophile that is introduced to the precursor RNA polynucleotide, e.g., not in cis and/or covalently linked to the precursor RNA polynucleotide. In some embodiments, the nucleophile is a guanosine that is capable of cleaving at the splice site dinucleotide at or near the 5′ end of the monotron. In some embodiments, the guanosine is a free guanosine that is introduced to the precursor RNA polynucleotide, e.g., not in cis and/or covalently linked to the precursor RNA polynucleotide. In some embodiments, the cleavage product of (iii) comprises a 5′ splice site nucleotide having a 3′ hydroxyl group that is capable of cleaving at the splice site nucleotide of the terminal element.

In some embodiments, the precursor RNA polynucleotide comprises, in the following order, (a) a monotron element; (b) an intervening region, and (c) terminal element. In some embodiments, the monotron sequence is upstream of the terminal sequence in the precursor RNA polynucleotide. In such embodiments: (i) the monotron element comprises a splice site dinucleotide at or near the 3′ end of the monotron, (ii) the terminal element comprises a splice site nucleotide, and (iii) the monotron element is capable of interacting with a nucleophile that is capable of cleaving at the splice site nucleotide of the terminal element, where the cleavage product of (iii) comprises a 5′ splice site nucleotide that is capable of cleaving at the splice site dinucleotide at or near the 3′ end of the monotron. In some embodiments, the nucleophile is a free nucleophile that is introduced to the precursor RNA polynucleotide, e.g., not in cis and/or covalently linked to the precursor RNA polynucleotide. In some embodiments, the nucleophile is a guanosine that is capable of cleaving at the splice site nucleotide of the terminal element. In some embodiments, the guanosine is a free guanosine that is introduced to the precursor RNA polynucleotide, e.g., not in cis and/or covalently linked to the precursor RNA polynucleotide. In some embodiments, the cleavage product of (iii) comprises a 5′ splice site nucleotide having a 3′ hydroxyl group that is capable of cleaving at the splice site nucleotide of the terminal element.

In some embodiments where the terminal sequence is upstream to the monotron, the monotron can perform two transesterification reactions. The monotron can (a) self-cleave and (b) form a phosphodiester bond with the terminal sequence. In some embodiments, the reactions (a) and (b) are sequential. In some embodiments, (a) the monotron is capable of interacting with a nucleophile that is capable of cleaving at the splice site dinucleotide at or near the 5′ end of the monotron, and (b) the cleavage product of (a), i.e., the 5′ splice site nucleotide, e.g., having a 3′ hydroxyl group, engages in a transesterification reaction (cleaves) at the splice site nucleotide of the terminal sequence, yielding a circular RNA or oRNA. In these embodiments, the monotron interacts with the nucleophile by forming a binding pocket with the nucleophile, and the linear precursor is capable of adopting a conformation in which the nucleophile is in proximity to and is capable of cleaving at the splice site dinucleotide at or near the 5′ end of the monotron. In some embodiments, the nucleophile can be a guanosine, e.g., a free guanosine that is introduced to the precursor RNA polynucleotide.

In some embodiments where the monotron sequence is upstream of the terminal sequence, the monotron can also perform two transesterification reactions. In some embodiments, (a) the monotron is capable of interacting with a nucleophile that is capable of cleaving at the splice site nucleotide of the terminal element, and (b) the cleavage product of (a), i.e., the 5′ splice site nucleotide, e.g., having a 3′ hydroxyl group, engages in a transesterification reaction (cleaves) at the splice site dinucleotide at or near the 3′ end of the monotron, yielding a circular RNA or oRNA. In these embodiments, the monotron interacts with the nucleophile by forming a binding pocket with the nucleophile, and the linear precursor is capable of adopting a conformation in which the nucleophile is in proximity to and is capable of cleaving the splice site nucleotide of the terminal element. In some embodiments, the nucleophile can be a guanosine, e.g., a free guanosine that is introduced to the precursor RNA polynucleotide.

In some embodiments, the monotron comprises a 5′ proximal end of a natural group I or group II intron including the splice site dinucleotide and optionally a natural exon sequence or fragment thereof. In some embodiments, the 5′ end of the monotron refers to nucleotides within the 5′ half of the monotron. In some embodiments, the 3′ end of the monotron refers to nucleotides within the 3′ half of the monotron. In some embodiments, at or near the 5′ end of the monotron refers to within the 5′ half of the monotron. In some embodiments, at or near the 5′ end of the monotron refers to within the first ten 5′ positions in the monotron. In some embodiments, at the 5′ end of the monotron refers to the first 5′ position(s) in the monotron. In some embodiments, at or near the 3′ end of the monotron refers to within the 3′ half of the monotron. In some embodiments, at or near the 3′ end of the monotron refers to within the last ten 3′ positions in the monotron. In some embodiments, at the 3′ end of the monotron refers to last 3′ position(s) in the monotron.

In some embodiments, the splice site nucleotide of the terminal element is not a natural splice site dinucleotide associated with a natural Group I or Group II intron sequence. In some embodiments, the terminal element comprises at least a portion of a natural exon or a fragment of a natural exon. In some embodiments, the natural exon is a Group I or Group II exon. In some embodiments, the natural exon or fragment thereof is 10-20 nucleotides in length. In some embodiments, the terminal element comprises a synthetic derivative of a natural exon or fragment thereof. In some embodiments, the terminal element comprises an exon or synthetic nucleotides that are longer than the splice site nucleotide that can help with splicing.

In some embodiments, the terminal element sequence has a percent sequence identity of about 70%, 75%, 80%, 85%, 90%, 91%, 92%, 93%, 94%, 95%, 96%, 97%, 98%, 99% or more to an exon fragment of a sequence selected from Tables A or B. In some embodiments, the terminal element sequence comprises an exon fragment comprising one, two, three, four, five, six, seven, eight, nine, ten, or more mutations to a sequence selected from Tables A or B. The mutations are, for example, selected from insertions, deletions, mutations, additions, and subtractions. In some embodiments, the terminal element or exon fragment thereof comprises a polynucleotide sequence selected from a sequence set forth in SEQ ID NOS: 2990-3668.

In some embodiments, the terminal element is less than 500, less than 450, less than 400, less than 350, less than 300, less than 250, less than 200, less than 150, less than 100, less than 90, less than 80, less than 70, less than 60, less than 50, less than 40, less than 30, less than 20, or less than 10 nucleotides in length.

In some embodiments, the terminal element is capable of directing or functionalizing the splicing activity of the monotron element.

In some embodiments, a portion of the terminal segment is retained upon circularization. In some embodiments, a portion of the terminal segment is excised upon circularization. In some embodiments, all or a portion of the terminal element is excised post-circularization. In some embodiments, the terminal element is not excised upon cleavage and is retained post-cleavage.

In some embodiments, the monotron element comprises at least a portion of a Group I or Group II intron. In some embodiments, Group I or Group II intron is selected from a genus and/or species described in Tables A or B. In some embodiments, the Group I or Group II intron is from a gene selected from Cyanobacterium Anabaena sp., T4 phage, Hypocrea pallida, Bulbithecium hyalosporum, Myoarachis inversa, Geosmithia argillacea, Coxiella burnetii, Agrobacterium tumefaciens, Azoarcus, Nostoc, Cordyceps capitata, Prochlorothrix hollandica, and Tilletiopsis orzyzicola. In some embodiments, the monotron element or a fragment thereof has a percent sequence identity of about 70%, 75%, 80%, 85%, 90%, 91%, 92%, 93%, 94%, 95%, 96%, 97%, 98%, 99% or more to a sequence selected from Tables A or B. In some embodiments, the monotron element sequence or fragment thereof comprises one, two, three, four, five, six, seven, eight, nine, ten, or more mutations to a sequence selected from Tables A or B. The mutations are, for example, selected from insertions, deletions, additions, and subtractions. In some embodiments the monotron element sequence or fragment thereof comprises a polynucleotide sequence having a percent sequence identity of about 70%, 75%, 80%, 85%, 90%, 91%, 92%, 93%, 94%, 95%, 96%, 97%, 98%, 99%, or more to a portion of a sequence set forth in SEQ ID NOS: 2990-3668.

In some embodiments, the Group I or Group II intron or introns, or portion thereof, are at least 10 nucleotides in length.

In some embodiments, the monotron element comprises at least one mutation of a native Group I intron-adjacent exon sequence or Group II intron-adjacent exon sequence. In some embodiments, the at least one mutation is at least one substation mutation of a native Group I intron-adjacent exon sequence or Group II intron-adjacent exon sequence. In some embodiments, the at least one mutation is at least one deletion of a native Group I intron-adjacent exon sequence or Group II intron-adjacent exon sequence. In some embodiments, at least one of the exon segments is less than 15 nucleotides in length. In some embodiments, the monotron element comprises a 3′ exon segment and/or 5′ exon segment, wherein the 3′ or 5′ exon segment comprises a Group I exon segment or a Group II exon segment less than 15 nucleotides in length. In some embodiments, the 3′ exon segments and/or 5′ exon segments, have a percent sequence identity of about 70%, 75%, 80%, 85%, 90%, 91%, 92%, 93%, 94%, 95%, 96%, 97%, 98%, 99% or more to a native Group I intron-adjacent exon sequence or Group II intron-adjacent exon sequence.

In some embodiments, the monotron element is less than 500 nucleotides in length.

In some embodiments, the monotron element is capable of inducing circularization when it interacts with the terminal element. In some embodiments, a portion of the monotron element is excised post-circularization. In some embodiments, the monotron element is fully excised post-circularization. In some embodiments, a portion of the monotron element and a portion of the terminal element are retained and excised post-circularization.

In some embodiments, the precursor RNA polynucleotide comprises at least one affinity tag or affinity sequence. Affinity sequences are described in further detail herein. In some embodiments, the affinity tag comprises a polyA sequence or is a polyA affinity tag. In some embodiments the terminal element comprises an affinity tag. In some embodiments, the terminal element comprises a 5′ affinity tag or a 3′ affinity tag. In some embodiments, the monotron element comprises an affinity tag. In some embodiments, the monotron element comprises a 3′ affinity tag or a 5′ affinity tag.

In some embodiments, the precursor RNA polynucleotide comprises an internal and/or external spacer. Spacers of the present disclosure are described in further detail herein. In some embodiments, the precursor RNA polynucleotide comprises an internal spacer sequence positioned between the terminal element and the intervening region. In some embodiments, the precursor RNA polynucleotide comprises an internal spacer sequence positioned between the intervening region and the monotron element. In some embodiments, the precursor RNA polynucleotide comprises an external spacer. In some embodiments, the external spacer is positioned adjacent to the terminal element. In some embodiments, the external spacer is positioned adjacent to the monotron element. In some embodiments, the precursor RNA polynucleotide comprises internal spacers and/or external spacers. The internal spacers and external spacers can each comprise an unstructured, structured or randomly generated polynucleotide sequence. In some embodiments, the internal spacers and external spacers are at least 5 nucleotides in length and can be about 5-60 nucleotides in length. In some embodiments, the internal and external spacers are 5-60 nucleotides in length, inclusive.

In some embodiments, the precursor RNA polynucleotide comprises one or more duplexes. Duplexes of the present disclosure are described in further detail herein. In some embodiments, the precursor RNA polynucleotide comprises a 5′ internal duplex sequence and a 3′ internal duplex sequence. In embodiments where the terminal element is upstream of the monotron element, the 5′ internal duplex sequence is positioned between the terminal element and the intervening region, and the 3′ internal duplex sequence is positioned between the intervening region and the monotron element. In embodiments where the monotron element is upstream of the terminal element, the 5′ internal duplex sequence is positioned between monotron and the intervening region, and the 3′ internal duplex sequence is positioned between the intervening region and the terminal element. In some embodiments, the 5′ internal duplex sequence and 3′ internal duplex sequence are at least 80% complementary. In some embodiments, a duplex is 3-100 nucleotides in length. In some embodiments, a duplex is 5-20 nucleotides in length, inclusive. In some embodiments, the 5′ and 3′ internal duplex sequences are capable of forming, and are predicted to form, a contiguous duplex. In some embodiments, the continuous duplex has a length of no longer than about 35 nucleotides. In some embodiments, the 5′ internal duplex sequence and/or 3′ internal duplex sequence each have a GC content of at least 10%.

In some embodiments, the precursor RNA polynucleotide comprises at least one affinity tag and at least one external spacer. In some embodiments, the precursor RNA polynucleotide comprises at least one internal duplex and at least one internal spacer, for example a 5′ affinity tag and 5′ internal spacer and/or a 3′ affinity tag and 3′ internal spacer. In embodiments where the polynucleotide comprises a 5′ affinity tag, the 5′ affinity tag is positioned adjacent to the 5′ external spacer, and in certain embodiments is positioned 5′ to the 5′ external spacer. In some embodiments where the polynucleotide comprises a 3′ affinity tag, the 3′ affinity tag is positioned adjacent to the 3′ external spacer, and in certain embodiments is positioned 3′ to the 3′ external spacer.

In some embodiments, the precursor RNA polynucleotide comprises at least one duplex and at least one internal spacer. In some embodiments, the precursor RNA polynucleotide comprises at least one internal duplex and at least one internal spacer, for example a 5′ internal duplex and a 5′ internal spacer and/or a 3′ internal duplex and a 3′ internal spacer. In some embodiments where the polynucleotide comprises a 5′ internal duplex, the 5′ internal duplex is positioned adjacent to the 5′ internal spacer, and in certain embodiments is positioned 5′ to the 5′ internal spacer. In some embodiments where the polynucleotide comprises a 3′ internal duplex, the 3′ internal duplex is positioned adjacent to the 3′ internal spacer, and in certain embodiments, the 3′ internal duplex is positioned 3′ to the 3′ internal spacer.

In some embodiments, the precursor polynucleotide comprises a 3′ and/or 5′ exon segment. In some embodiments, at least a portion of the 3′ and/or 5′ exon segment is codon optimized.

In some embodiments, the precursor RNA polynucleotide described above further comprises a leading untranslated sequence and/or a lagging untranslated sequence. For example, the precursor RNA polynucleotide can comprise a 5′ external spacer that is positioned between a leading untranslated sequence and the terminal element if the terminal element is upstream of the monotron element; or between a leading untranslated sequence and the monotron element if the monotron element is upstream of the terminal element. In some embodiments, the precursor RNA polynucleotide comprises a 3′ external spacer that is positioned between the monotron element and a lagging untranslated sequence if the terminal element is upstream of the monotron element; or between the terminal element and a lagging untranslated sequence if the monotron element is upstream of the terminal element.

As described in detail elsewhere herein, the intervening region of the precursor RNA polynucleotide can comprise sequences directed to, for example, an aptamer, a coding element, a stop codon or stop cassette, an intervening region comprising an untranslated region, a noncoding element. As set forth in detail herein, the intervening region can comprise a coding element where the coding element comprises, for example, a sequence encoding a therapeutic protein. In some embodiments, the intervening region comprises an untranslated region, which can comprise one or more non-coding element, including but not limited to, a natural 5′ Untranslated Region (UTR), a natural 3′ Untranslated Region (UTR), a synthetic spacer sequence, an aptamer, TIE, a viral or eukaryotic IRES, or sequences selected from, e.g., lncRNA, miRNA, or a miRNA sponge.

In some embodiments, the polynucleotide comprises at least one mutation of a native Group I intron-adjacent exon sequence or Group II intron-adjacent exon sequence. The mutation can be, for example, at least one mutation of a native Group I or native Group II intron-adjacent exon sequence. For example, the mutation can be one substitution, at least one deletion, and/or at least one insertion of a native Group I or Group II intron-adjacent exon sequence. In some embodiments, at least one of the exon segments is less than 15 nucleotides in length. In some embodiments, the 3′ exon segment and/or 5′ exon segment comprises a Group I exon segment or a Group II exon segment. In some embodiments, the at least one exon segment is less than 15 nucleotides in length. In some embodiments, the at least one exon segment has a percent sequence identity of about 70%, 75%, 80%, 85%, 90%, 91%, 92%, 93%, 94%, 95%, 96%, 97%, 98%, 99% or more to a native Group I intron-adjacent exon sequence or Group II intron-adjacent exon sequence. In some embodiments, the at least one segment is selected from a 3′ exon segment, 5′ exon segment, or a 3′ and 5′ permuted exon segment. In some embodiments, the exon sequence or fragment is in the terminal element.

Also provided herein are polynucleotides encoding the precursor RNA polynucleotides described above that comprise a monotron and terminal element. Polynucleotides, for example, DNA templates comprising sequences encoding the precursor RNAs described above, and their uses in related methods are described elsewhere herein. Polynucleotides of the present disclosure can comprise, for example, an expression vector, DNA plasmid, a cosmid, a PCR product, dbDNA close-ended DNA (ceDNA), and a viral polynucleotide. In some embodiments, the polynucleotides can comprises a promoter segment, for example a T7 promoter, SP6 promoter or a fragment thereof.

Also provided herein are circular RNA polynucleotides produced by the precursor RNAs described above that comprise a monotron and terminal element. Circular RNAs are described in detail elsewhere herein. In some embodiments, a circular RNA polynucleotide comprises: at least a portion of a terminal element, an intervening region, and at least a portion of a monotron element. In some embodiments, a circular RNA comprises: (a) at least a portion of a terminal element, (b) a 3′ exon segment comprising a 3′ nucleotide of a 3′ splice site dinucleotide, (c) an intervening region, (d) a 5′ exon segment comprising a 5′ nucleotide of a 5′ splice site dinucleotide, and (e) at least a portion of a monotron element. In some embodiments, (d) comprises the first nucleotide of a 5′ Group I or Group II splice site dinucleotide and a natural exon sequence and (b) comprises the second nucleotide of a 3′ Group I or Group II exon splice site dinucleotide and a natural exon sequence. In some embodiments, the 5′ and/or 3′ splice site dinucleotides are distinct from the natural splice site dinucleotide(s) associated with a natural Group I or Group II intron sequence. In some embodiments, the circular RNA polynucleotides comprise additional elements, including but not limited to, a 5′ internal duplex and/or 3′ internal duplex; a 5′ internal spacer and/or 3′ internal spacer. In some embodiments, the circular RNA polynucleotide is from about 50 nucleotides to about 15 kilobases in length.

Related cells comprising the precursor RNA polynucleotides, delivery or transfer vehicles, and pharmaceutical compositions thereof are described elsewhere herein in further detail. Related methods of producing circularized RNA and related methods of treating a subject in need thereof are also provided herein.

Also provided herein are methods of identifying a monotron element and terminal element pair that allows production of a circular RNA that is translatable or biologically active inside a eukaryotic cell, comprising, for example: (i) inserting a mutated 5′ and 3′ Group I or Group II intron sequence derived from a database of native intronic sequence to form a monotron element into a precursor RNA polynucleotide described above; (ii) inserting a synthetic polynucleotide sequence to form a terminal element into a precursor RNA polynucleotide described above; (iii) transcribing the polynucleotide into RNA in vitro or allowing the polynucleotide to be transcribed into RNA by a cell; and (iv) determining the circularization efficiency of the RNA produced by the polynucleotide by identifying the amount of circularized RNA, the amount of excised intronic sequences, the amount of precursor RNA remaining after circularization, and combinations thereof. In some embodiments, the mutated 5′ and 3′ Group I or Group II intron sequence comprises at least one deletion, insertion or substitution of at least one nucleotide. In some embodiments, the 5′ or 3′ Group I or Group II intronic sequences, or combinations thereof are sequenced.

Also provided herein are methods for determining a polynucleotide sequence that improves RNA circularization efficiency compared to a polynucleotide comprising a native intronic sequence or to a parent polynucleotide with a known sequence, the method comprising modifying a DNA sequence encoding the precursor RNA polynucleotide described above, the modifying comprising: (i) mutating at least one nucleotide and/or altering the length of the terminal element and/or monotron element of the DNA sequence encoding the precursor RNA polynucleotide described above; (ii) altering the length of the 5′ and/or 3′ internal and/or external spacer sequence of the DNA sequence encoding precursor RNA polynucleotide described above; (iii) altering the length of the 5′ and/or 3′ internal duplex sequence of the DNA sequence encoding the precursor RNA polynucleotide described above; (iv) altering the length of the 5′ and/or 3′ exon sequence of the DNA sequence encoding the precursor RNA polynucleotide described above; (iv) or combinations thereof; and transcribing the polynucleotide comprising the DNA sequence into RNA in vitro or allowing the polynucleotide comprising the DNA sequence to be transcribed into RNA by a cell; and determining the circularization efficiency of the RNA produced by the polynucleotide comprising the DNA sequence by identifying the amount of circularized RNA, the amount of excised intronic sequences, the amount of precursor RNA remaining after circularization, and combinations thereof. In some embodiments, the methods further comprise comparing the circularization efficiency of the polynucleotide with a polynucleotide comprising a native intronic sequence, or a parent polynucleotide.

### C. Intervening Region

In various embodiments, a provided polynucleotide (e.g., a DNA template, a linear precursor RNA polynucleotide, or a circular RNA polynucleotide) comprises an intervening region.

a. Coding or Noncoding Element

In some embodiments, the intervening region and/or core functional element comprises one or more noncoding elements, e.g., microRNA binding site, IRES transacting factor region, restriction site, a RNA editing region, structural or sequence element, a granule site, a zip code element, or an RNA trafficking element. In some embodiments, the intervening region and/or core functional element comprises one or more coding elements. In some embodiments, the intervening region and/or core functional element comprises a combination of coding and noncoding elements. In some embodiments, the coding or non-coding region is a part of the core functional element or intervening region located between the 5′ end and 3′ end of the linear precursor RNA polynucleotide and resultant circular RNA.

In some embodiments, the coding element comprises an expression sequence. In some embodiments, the coding element comprises a sequence encoding at least one therapeutic protein. In some embodiments, the coding element encodes two or more polypeptides. In some embodiments, the sequences encoding the two or more polypeptides are separated by a ribosomal skipping element or a nucleotide sequence encoding a protease cleavage site. In certain embodiments, the ribosomal skipping element encodes thosea-asigna virus 2A peptide (T2A), porcine teschovirus-1 2 A peptide (P2A), foot-and-mouth disease virus 2 A peptide (F2A), equine rhinitis A vims 2A peptide (E2A), cytoplasmic polyhedrosis vims 2A peptide (BmCPV 2A), or flacherie vims of B. mori 2A peptide (BmIFV 2A). Coding elements or regions and payloads are described in further detail elsewhere herein.

In some embodiments, the intervening region comprises at least one translation initiation element (TIE). TIEs are designed to allow translation efficiency of an encoded protein. In some embodiments, core functional elements comprising one or more coding elements will further comprise one or more TIEs. In some embodiments, a translation initiation element (TIE) comprises a synthetic TIE. In some embodiments, a synthetic TIE comprises aptamer complexes, synthetic IRES or other engineered TIEs capable of initiating translation of a linear RNA or circular RNA polynucleotide.

In some embodiments, the intervening region comprises one or more noncoding elements. In some embodiments, the noncoding element comprises an untranslated region (UTR) or fragment thereof. In some embodiments, the noncoding element is a natural 5′ UTR. In some embodiments, the noncoding element is a natural 3′ UTR. In some embodiments, the noncoding element is a synthetic spacer sequence. In some embodiments, the noncoding element is an aptamer. In some embodiments, the noncoding element is or comprises a translation initiation element (TIE). In some embodiments, the noncoding element comprises a lncRNA, miRNA, or a miRNA sponge.

In some embodiments, the intervening region comprises a TIE comprising an untranslated region (UTR) or a fragment thereof, an aptamer complex or a fragment thereof, or a combination thereof. In certain embodiments, the TIE contains modified nucleotides.

In certain embodiments, the TIE provided herein comprise an internal ribosome entry site (IRES). In certain embodiments, the TIE provided herein comprise a viral or eukaryotic internal ribosome entry site (IRES) or a fragment or variant thereof. In certain embodiments, the IRES comprises one or more modified nucleotides compared to the wild-type viral IRES or eukaryotic IRES. See, e.g., PCT Application No. US2022/33091, which is incorporated herein by reference in its entirety.

In some embodiments, the noncoding element comprises an untranslated region (UTR). In some embodiments, the noncoding element is a natural 5′ UTR. In some embodiments, the noncoding element is a natural 3′ UTR. In some embodiments, the noncoding element is a synthetic spacer sequence. In some embodiments, the noncoding element is an aptamer or synthetic aptamer. In some embodiments, the noncoding element is or comprises a translation initiation element (TIE).

b. Translation Initiation Element

In some embodiments, the DNA template, linear precursor RNA polynucleotide, and circular RNA polynucleotide comprise an intervening region and/or core functional element. In some embodiments, the intervening region and/or core functional element comprises a coding and/or noncoding element. In some embodiments, the intervening region and/or core functional element further comprises a translation initiation element (TIE) upstream to the coding or noncoding element, and/or a termination element.

In some embodiments, the polynucleotide comprises a translation initiation element (TIE). In some embodiments, the intervening region comprises at least one TIE. In some embodiments, the TIE is upstream to a coding or noncoding element. In some embodiments, TIEs are designed to allow translation efficiency of an encoded protein. Accordingly, in some embodiments, an intervening region comprising one or more coding elements further comprises one or more TIEs. In other embodiments, an intervening region comprising only noncoding elements lacks any TIEs.

In some embodiments, a TIE comprises an internal ribosome entry site (IRES). In certain embodiments, the TIE provided herein comprise a viral or eukaryotic internal ribosome entry site (IRES) or a fragment or variant thereof. In some embodiments, inclusion of an IRES permits the translation of one or more open reading frames from a circular RNA (e.g., open reading frames that form the expression sequences). In some embodiments, IRES attracts a eukaryotic ribosomal translation initiation complex and promotes translation initiation. See, e.g., PCT Application No. WO202261490, which is incorporated herein by reference in its entirety.

i. Natural TIES: Viral & Eukaryotic Cellular IRES

In certain embodiments, as provided herein, the payload encoded by the circular RNA polynucleotide may be optimized through use of a specific internal ribosome entry sites (IRES) within the translation initiation element (TIE). In some embodiments, IRES specificity within a circular RNA can significantly enhance expression of specific proteins encoded within the coding element. In some embodiments, the IRES comprises a viral IRES or eukaryotic IRES.

A multitude of IRES sequences are available and include sequences derived from a wide variety of viruses, such as from leader sequences of picornaviruses such as the encephalomyocarditis virus (EMCV) UTR (Jang et al., J. Virol. (1989) 63: 1651-1660), the polio leader sequence, the hepatitis A virus leader, the hepatitis C virus IRES, human rhinovirus type 2 IRES (Dobrikova et al., Proc. Natl. Acad. Sci. (2003) 100(25): 15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279(5):3389-3397), and the like.

Inclusion of an IRES permits the translation of one or more open reading frames from a circular RNA (e.g., open reading frames that form the expression sequences). The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20: 102-110; Kobayashi et al., BioTechniques (1996) 21:399-402; and Mosser et al., BioTechniques 1997 22 150-161. In some embodiments, the IRES is capable of facilitating expression of a protein encoded by the precursor RNA in a cell. In some embodiments, the IRES is capable of facilitating expression of the protein, such that the expression level of the protein is comparable to or higher than when a control IRES is used.

Different IRES sequences have varying ability to drive protein expression, and the ability of any particular identified or predicted IRES sequence to drive protein expression from linear mRNA or circular RNA constructs is unknown and unpredictable. In certain embodiments, potential IRES sequences can be bioinformatically identified based on sequence positions in viral sequences. However, the activity of such sequences has been previously uncharacterized. As demonstrated herein, such IRES sequences may have differing protein expression capability depending on cell type, for example in T cells, liver cells, or muscle cells. In some embodiments, the novel IRES sequences described herein may have at least 2, 3, 4, 5, 6, 7, 8, 9, 10, 20, 50, or 100 fold increased expression in a particular cell type compared to previously described EMCV IRES sequences.

In some embodiments, for driving protein expression, a polynucleotide (e.g., a DNA template, a linear precursor RNA polynucleotide, or a circular RNA polynucleotide) comprises an IRES operably linked to a protein coding sequence. In some embodiments, the IRES comprises a sequence selected from the sequences in Table 1 or a fragment thereof or a sequence selected from SEQ ID NOS: 1-2989 and 4045-25570 (GIRES 0-10762), or a fragment thereof. In some embodiments, the IRES comprises a sequence at least 80%, 85%, 90%, 91%, 92%, 93%, 94%, 95%, 96%, 97%, 98%, or 99% identical to a sequence selected from the sequences in Table 1 or a sequence selected from SEQ ID NOS: 1-2989 and 4045-25570 (GIRES 0-10762), or a fragment thereof. See also, e.g., PCT Application No. US2022/33091 (WO202261490), which is incorporated herein by reference in its entirety.

In some embodiments, the IRES is derived from Aalivirus, Ailurivirus, Ampivirus, Anativirus, Aphthovirus, Aquamavirus, Avihepatovirus, Avisivirus, Boosepivirus, Bopivirus, Caecilivirus, Cardiovirus, Cosavirus, Crahelivirus, Crohivirus, Danipivirus, Dicipivirus, Diresapivirus, Enterovirus, Erbovirus, Felipivirus, Fipivirus, Gallivirus, Gruhelivirus, Grusopivirus, Harkavirus, Hemipivirus, Hepatovirus, Hunnivirus, Kobuvirus, Kunsagivirus, Limnipivirus, Livupivirus, Ludopivirus, Malagasivirus, Marsupivirus, Megrivirus, Mischivirus, Mosavirus, Mupivirus, Myrropivirus, Orivirus, Oscivirus, Parabovirus, Parechovirus, Pasivirus, Passerivirus, Pemapivirus, Poecivirus, Potamipivirus, Pygoscepivirus, Rabovirus, Rafivirus, Rajidapivirus, Rohelivirus, Rosavirus, Sakobuvirus, Salivirus, Sapelovirus, Senecavirus, Shanbavirus, Sicinivirus, Symapivirus, Teschovirus, Torchivirus, Tottorivirus, Tremovirus, Tropivirus, Hepacivirus, Pegivirus, Pestivirus, or Flavivirus. In some embodiments herein, the IRES is selected from an Enterovirus, Kobuvirus, Parechovirus, Hunnivirus, Passerivirus, Mischivirus, and Cardiovirus.

In some embodiments, the TRES is an IRES sequence derived from Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Himetobi P virus, Hepatitis C virus, Hepatitis A virus, Hepatitis GB virus, Foot and mouth disease virus, Human enterovirus 71, Equine rhinitis virus, Ectropis obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAPI, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SH1, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picorna-like Virus, CRPV, Salivirus A BN5, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24, or an aptamer to eIF4G.

In some embodiments, the IRES comprises in whole or in part a eukaryotic or cellular IRES. In certain embodiments, the IRES is an IRES sequence derived from a human gene, wherein the human gene is ABCF1, ABCG01, ACAD10, ACOT7, ACSS3, ACTG2, ADCYAP1, ADK, AGTR1, AHCYL2, AHI1, AKAP8L, AKR1A1, ALDH3A1, ALDOA, ALG13, AMMECR1L, ANGPTL4, ANK3, AOC3, AP4B1, AP4E1, APAF1, APBB1, APC, APH1A, APOBEC3D, APOM, APP, AQP4, ARHGAP36, ARL13B, ARMC8, ARMCX6, ARPC1A, ARPC2, ARRDC3, ASAP1, ASB3, ASB5, ASCL1, ASMTL, ATF2, ATF3, ATG4A, ATP5B, ATP6VOA1, ATXN3, AURKA, AURKA, AURKA, AURKA, B3GALNT1, B3GNTL1, B4GALT3, BAAT, BAG1, BAIAP2, BAIAP2L2, BAZ2A, BBX, BCAR1, BCL2, BCS1L, BET1, BID, BIRC2, BPGM, BPIFA2, BRINP2, BSG, BTN3A2, C12orf43, C14orf93, C17orf62, Clorf226, C21orf62, C2orfl5, C4BPB, C4orf22, C9orf84, CACNA1A, CALCOCO2, CAPN11, CASP12, CASP8AP2, CAV1, CBX5, CCDC120, CCDC17, CCDC186, CCDC51, CCN1, CCND1, CCNT1, CD2BP2, CD9, CDC25C, CDC42, CDC7, CDCA7L, CDIP1, CDK1, CDK11A, CDKN1B, CEACAM7, CEP295NL, CFLAR, CHCHD7, CHIA, CHIC1, CHMP2A, CHRNA2, CLCN3, CLEC12A, CLEC7A, CLECL1, CLRN1, CMSS1, CNIH1, CNR1, CNTN5, COG4, COMMD1, COMMD5, CPEB1, CPS1, CRACR2B, CRBN, CREM, CRYBG1, CSDE1, CSF2RA, CSNK2A1, CSTF3, CTCFL, CTH, CTNNA3, CTNNB1, CTNNB1, CTNND1, CTSL, CUTA, CXCR5, CYB5R3, CYP24A1, CYP3A5, DAG1, DAP3, DAP5, DAXX, DCAF4, DCAF7, DCLRElA, DCP1A, DCTN1, DCTN2, DDX19B, DDX46, DEFB123, DGKA, DGKD, DHRS4, DHX15, DI03, DLG1, DLL4, DMD UTR, DMD ex5, DMKN, DNAH6, DNAL4, DUSP13, DUSP19, DYNC1I2, DYNLRB2, DYRKIA, ECI2, ECT2, EIF1AD, EIF2B4, EIF4G1, EIF4G2, EIF4G3, ELANE, ELOVL6, ELP5, EMCN, ENOl, EPB41, ERMN, ERVV-1, ESRRG, ETFB, ETFBKMT, ETV1, ETV4, EXD1, EXT1, EZH2, FAM111B, FAM157A, FAM213A, FBXO25, FBXO9, FBXW7, FCMR, FGF1, FGF1, FGF1A, FGF2, FGF2, FGF-9, FHLS, FMR1, FN1, FOXP1, FTH1, FUBP1, G3BP1, GABBR1, GALC, GART, GAS7, gastrin, GATA1, GATA4, GFM2, GHR, GJB2, GLI1, GLRA2, GMNN, GPAT3, GPATCH3, GPR137, GPR34, GPR55, GPR89A, GPRASP1, GRAP2, GSDMB, GSTO2, GTF2B, GTF2H4, GUCY1B2, HAX1, HCST, HIGD1A, HIGD1B, HIPK1, HIST1H1C, HIST1H3H, HK1, HLA-DRB4, HMBS, HMGA1, HNRNPC, HOPX, HOXA2, HOXA3, HPCAL1, HR, HSP90AB1, HSPA1A, HSPA4L, HSPA5, HYPK, IFFO1, IFT74, IFT81, IGF1, IGF1R, IGF1R, IGF2, ILI1, IL17RE, IL1RL1, IL1RN, IL32, IL6, ILF2, ILVBL, INSR, INTS13, IP6K1, ITGA4, ITGAE, KCNE4, KERA, KIAA0355, KIAA0895L, KIAA1324, KIAA1522, KIAA1683, KIF2C, KIZ, KLHL31, KLK7, KRR1, KRT14, KRT17, KRT33A, KRT6A, KRTAP10-2, KRTAP13-3, KRTAP13-4, KRTAP5-11, KRTCAP2, LACRT, LAMB1, LAMB3, LANCL1, LBX2, LCAT, LDHA, LDHAL6A, LEF1, LINC-PINT, LMO3, LRRC4C, LRRC7, LRTOMT, LSM5, LTB4R, LYRM1, LYRM2, MAGEA11, MAGEA8, MAGEBI, MAGEB16, MAGEB3, MAPT, MARS, MC1R, MCCC1, METTL12, METTL7A, MGC16025, MGC16025, MIA2, MIA2, MITF, MKLN1, MNT, MORF4L2, MPD6, MRFAP1, MRPL21, MRPS12, MSI2, MSLN, MSN, MT2A, MTFR1L, MTMR2, MTRR, MTUS1, MYB, MYC, MYCL, MYCN, MYL10, MYL3, MYLK, MYO1A, MYT2, MZB1, NAP1L1, NAV1, NBAS, NCF2, NDRG1, NDST2, NDUFA7, NDUFB11, NDUFC1, NDUFS1, NEDD4L, NFAT5, NFE2L2, NFE2L2, NFIA, NHEJ1, NHP2, NIT1, NKRF, NME1-NME2, NPAT, NR3C1, NRBF2, NRF1, NTRK2, NUDCD1, NXF2, NXT2, ODC1, ODF2, OPTN, OR10R2, OR11L1, OR2M2, OR2M3, OR2M5, OR2T10, OR4C15, OR4F17, OR4F5, OR5H1, OR5K1, OR6C3, OR6C75, OR6N1, OR7G2, p53, P2RY4, PAN2, PAQR6, PARP4, PARP9, PC, PCBP4, PCDHGC3, PCLAF, PDGFB, PDZRN4, PELO, PEMT, PEX2, PFKM, PGBD4, PGLYRP3, PHLDA2, PHTF1, PI4 KB, PIGC, PIM1, PKD2L1, PKM, PLCB4, PLD3, PLEKHA1, PLEKHBI, PLS3, PML, PNMA5, PNN, POC1A, POC1B, POLD2, POLD4, POU5F1, PPIG, PQBP1, PRAME, PRPF4, PRR11, PRRT1, PRSS8, PSMA2, PSMA3, PSMA4, PSMD11, PSMD4, PSMD6, PSME3, PSMG3, PTBP3, PTCH1, PTHLH, PTPRD, PUS7L, PVRIG, QPRT, RAB27A, RAB7B, RABGGTB, RAET1E, RALGDS, RALYL, RARB, RCVRN, REG3G, RFC5, RGL4, RGS19, RGS3, RHD, RINL, RIPOR2, RITA1, RMDN2, RNASE1, RNASE4, RNF4, RPA2, RPL17, RPL21, RPL26L1, RPL28, RPL29, RPL41, RPL9, RPS11, RPS13, RPS14, RRBP1, RSU1, RTP2, RUNX1, RUNX1T1, RUNX1T1, RUNX2, RUSC1, RXRG, S100A13, S100A4, SAT1, SCHIP1, SCMH1, SEC14L1, SEMA4A, SERPINA1, SERPINB4, SERTAD3, SFTPD, SH3D19, SHC1, SHMT1, SHPRH, SIM1, SIRT5, SLC11A2, SLC12A4, SLC16A1, SLC25A3, SLC26A9, SLC5A11, SLC6A12, SLC6A19, SLC7A1, SLFN11, SLIRP, SMAD5, SMARCADI, SMN1, SNCA, SNRNP200, SNRPB2, SNX12, SOD1, SOX13, SOX5, SP8, SPARCL1, SPATA12, SPATA31C2, SPN, SPOP, SQSTM1, SRBD1, SRC, SREBF1, SRPK2, SSB, SSB, SSBP1, ST3GAL6, STAB1, STAMBP, STAU1, STAU1, STAU1, STAU1, STAU1, STK16, STK24, STK38, STMN1, STX7, SULT2B1, SYK, SYNPR, TAF1C, TAGLN, TANK, TAS2R40, TBC1D15, TBXAS1, TCF4, TDGF1, TDP2, TDRD3, TDRD5, TESK2, THAP6, THBD, THTPA, TIAM2, TKFC, TKTL1, TLR10, TM9SF2, TMC6, TMCO2, TMED10, TMEM116, TMEM126A, TMEM159, TMEM208, TMEM230, TMEM67, TMPRSS13, TMUB2, TNFSF4, TNIP3, TP53, TP53, TP73, TRAF1, TRAK1, TRIM31, TRIM6, TRMT1, TRMT2B, TRPM7, TRPM8, TSPEAR, TTC39B, TTLL11, TUBB6, TXLNB, TXNIP, TXNL1, TXNRD1, TYROBP, U2AF1, UBA1, UBE2D3, UBE2I, UBE2L3, UBE2V1, UBE2V2, UMPS, UNG, UPP2, USMG5, USP18, UTP14A, UTRN, UTS2, VDR, VEGFA, VEGFA, VEPH1, VIPAS39, VPS29, VSIG10L, WDHD1, WDR12, WDR4, WDR45, WDYHV1, WRAP53, XIAP, XPNPEP3, YAP1, YWHAZ, YY1AP1, ZBTB32, ZNF146, ZNF250, ZNF385A, ZNF408, ZNF410, ZNF423, ZNF43, ZNF502, ZNF512, ZNF513, ZNF580, ZNF609, ZNF707, or ZNRD1.

In some embodiments, the cell is a myotube. In some embodiments, the IRES is derived from Bopivirus, Oscivirus, Hunnivirus, Passerivirus, Mischivirus, Kobuvirus, Enterovirus, Cardiovirus, Salivirus, Rabovirus, Parechovirus, Gallivirus, or Sicinivirus. In some embodiments, the IRES is derived from Hunnivirus, Passerivirus, Kobuvirus, Bopivirus, or Enterovirus. In some embodiments, the IRES is derived from Enterovirus I, Enterovirus F, Enterovirus E, Enterovirus J, Enterovirus C, Enterovirus A, Enterovirus B, Aichivirus B, Parechovirus A, Cardiovirus F, Cardiovirus B, or Cardiovirus E.

In some embodiments, the cell is a hepatocyte. In some embodiments, the IRES is derived from Enterovirus, Bopivirus, Mischivirus, Gallivirus, Oscivirus, Cardiovirus, Kobuvirus, Rabovirus, Salivirus, Parechovirus, Hunnivirus, Tottorivirus, Passerivirus, Cosavirus, or Sicinivirus. In some embodiments, the IRES is derived from Enterovirus, Mischivirus, Kobuvirus, Bopivirus, or Gallivirus. In some embodiments, the IRES is derived from Enterovirus B, Enterovirus A, Enterovirus D, Enterovirus J, Enterovirus C, Rhinovirus B, Enterovirus H, Enterovirus I, Enterovirus E, Enterovirus F, Aichivirus B, Aichivirus A, Parechovirus A, Cardiovirus F, Cardiovirus E, or Cardiovirus B.

In some embodiments, the cell is a T cell. In some embodiments, the IRES is derived from Passerivirus, Bopivirus, Hunnivirus, Mischivirus, Enterovirus, Kobuvirus, Rabovirus, Tottorivirus, Salivirus, Cardiovirus, Parechovirus, Megrivirus, Allexivirus, Oscivirus, or Shanbavirus. In some embodiments, the IRES is derived from Passerivirus, Hunnivirus, Mischivirus, Enterovirus, or Kobuvirus. In some embodiments, the IRES is derived from Enterovirus I, Enterovirus D, Enterovirus C, Enterovirus A, Enterovirus J, Enterovirus H, Aichivirus B, Parechovirus A, or Cardiovirus B.

In some embodiments, for driving protein expression, a provided circular RNA comprises an IRES operably linked to a protein coding sequence. In some embodiments, the IRES comprises a sequence selected from SEQ ID NOS: 1-2989 and 4045-25570 (GIRES 0-10762) or Table 1 below, or SEQ ID NOs: 1-2983 and 3282-3287 of PCT Application No. US2022/33091 (WO202261490) or a fragment thereof. In some embodiments, the IRES comprises a sequence at least 80%, 85%, 90%, 91%, 92%, 93%, 94%, 95%, 96%, 97%, 98%, or 99% identical to a sequence selected from SEQ ID NOS: 1-2989 and 4045-25570 or Table 1 below, or a sequence selected from SEQ ID NOs: 1-2983 and 3282-3287 of PCT Application No. US2022/33091 (WO202261490). In some embodiments, the circular RNA disclosed herein comprises an IRES sequence at least 80%, 85%, 90%, 91%, 92%, 93%, 94%, 95%, 96%, 97%, 98%, or 99% identical to a sequence selected from SEQ ID NOS: 1-2989 and 4045-25570 or Table 1 below, or a sequence selected from SEQ ID NOs: 1-2983 and 3282-3287 of PCT Application No. US2022/33091 (WO202261490). In some embodiments, the circular RNA disclosed herein comprises an IRES sequence selected from SEQ ID NOS: 1-2989 and 4045-25570 or Table 1 below or a fragment thereof, or SEQ ID NOs: 1-2983 and 3282-3287 of PCT Application No. US2022/33091 (WO202261490) or a fragment thereof.

Further exemplary IRES sequences are provided in Table 1. In some embodiments, the precursor RNA polynucleotide, circular RNA constructs and related pharmaceutical compositions disclosed herein comprise an IRES sequence that is at least 80%, 85%, 90%, 91%, 92%, 93%, 94%, 95%, 96%, 97%, 98%, or 99% identical to an IRES sequence in Table 1. In some embodiments, the precursor RNA polynucleotide, circular RNA constructs and related pharmaceutical compositions disclosed herein comprise an IRES sequence in Table 1.

TABLE 1
IRES Sequences

| SEQ ID NO: | Sequence |
|---|---|
| 4000 | TTAAAACAGCTCTGGGGTTGTTCCCACCCCAGAGGCCCACGTGGCGGCCAG |
| | TACTCCGGTATTACGGTACCCTTGTACGCCTGTTTTATACTCCCTTCCCCTGT |
| | AACTTAGAAGCATACAAACCAAGTTCAATAGAAGGGGGTACAAACCAGTA |
| | CCACCACGAACAAGCACTCCTGTTTCCCCGGTGACATTGCATAGACTGTAC |
| | CCACGGTTGAAAGCGATCGATCCGTTACCCGCTCCTGTACTTCGAGAAGCC |
| | TAGTATCATCTTGGAATCTTCGATGCGTTGCGCTCAGCACTCAACCCCAGAG |
| | TGTAGCTTAGGCTGATGAGTCTGGACGTCCCCCACCGGCGACGGTGGTCCA |
| | GGCTGCGTTGGCGGCCTACCTGTGGCCCAAAGCCACAGGACGCTAGTTGTG |
| | AACAAGGTGTGAAGAGCCTATTGAGCTACAAGAGAGTCCTCCGGCCCCTGA |
| | ATGCGGCTAATCCTAACCACGGAGCAGGCAGTTGCAAACCAGCAACCGGC |
| | CTGTCGTAACGCGCAAGTCTGTGGCGGAACCGACTACTTTGGGTGTCCGTG |
| | TTTCCTTTTATTTTTACAATGGCTGCTTATGGTGACAATCATAGATTGTTATC |
| | ATAAAGCGACTTGGATTGGCCATCCGGTGAAAGTAAAACACATTGTTTACT |
| | TGTTTGTTGGATTCACTCCAATTAACACTTTTACTTACAAACTCATTACAAC |
| | AACTCTATTAATTAGAGATAAGCATCACA |
| 4001 | TTTCCCCTGTTCGTAACTAAGTGTGTGCCCAATCTCCTCACTCCTGCTGGCTT |
| | CACCGACCGGCAGTGTCCAAAATGCTAGGTGAATCCCCTCCCTTTCCTCTGG |
| | GCTTCTGCCCAGCTTCCTCCCCCCAGCCTGACGTGACACAGGCTGTGCAAA |
| | GACCCCGCGAAAGCTGCCAAAAGTGGCAATTGTGGGTCCCCCCTTTGTAAA |
| | GGCGTCGAGTCTTTCTCCCTCAAGGCTAGACCCGTCAGTGAATTCTGTCGGG |
| | CAACTAGTGACGCCACTGCACGCCTCTGACCTCGGCCGCGGAGTGCTGCCC |
| | CCCAAGTCGTGCCCCTGACCACAAGTTGTGCTGTCTGGCAAACATTGTCTGT |
| | GAGAATGTTCCGCTGTGGCTGCCAAGCCTGGCAACAGGCTGCCCCAGTGTG |
| | CGTAGTTCTCATCCAGACTTCGGTCTGGCAACTTGCTGTTAAGACACGGCGT |
| | AAGGGGCGTGTGCCAACGCCCTGGAACGAGTGTCCACTCTAATACCCCGAG |
| | GAATGCTACGCAGGTACCCCTGGTTCGCCAGGGATCTGAGCGTAGGCTAAT |
| | TGTCTAAGGGTATTTTCATTTCCCATTCTTTCTTTCTTGTTCATA |
| 4002 | TCCCCGGCATGAGAGGAATAGACTCTTTCAGGGTTGAAGCCACGAGTGTCG |
| | TTACCCGCACTGGTACTACGCAAAGCCTAGTAACATCTTGAAACTCTTTTTG |
| | GTTGGTCGTTCCACTAGTTACCCCCTAGTAGACCTGGCAGATGAGGCAGGA |
| | CGCTCCCCACTGGCGACAGTGGTCCTGCCTGCGTGGCTGCCTGCACACCCTT |
| | CGGGGTGTGAAGCCAAAAGAAAGACAAGGTGTGAAGAGCCCCGTGTGCTA |
| | CCAGTGAATCCTCCGGCCCCTGAATGCGGCTAATCTTACCCCACAGCTATTG |
| | CACACAATCCAGTGTGTATGTAGTCGTAATGAGCAATTGTGGGACGGAACC |
| | GACTACTTTGGGTGTCCGTGTTTCCTTTTATTCCCATGTTTCTGCTTATGGTG |
| | ACAATACTGACGTATAGTGTTGTTACC |
| 4003 | TTAAAACAGCGGATGGGTATCCCACCATCCGGCCCACTGGGTGTAGTACTC |
| | TGGTACATTGTACCTTTGTACGCCTGTTTTCCCCCTCTTGTACCCGCCCTTCA |
| | AGCTCCTTGCCCAAGTAACGTTAGAAGTTTGAACATTGGTACAATAGGAAG |
| | CATCACATCCAGTGGTGTACTGTACAAACACTTCTGTTGCCCCGGAGCGAG |
| | GTATAGATGGTCCCCACCGTCAAAAGCCTTTAACCGTTATCCGCCAATCAA |
| | CTACGTAATGGCTAGTAGCACCTTGGATTTAAGTTGGCGTTCGATCAGGTG |
| | GTAACCCCCACTAGTTTGGTCGATGAGGCTAGGAATTCCCCACGGGTGACC |
| | GTGTCCTAGCCTGCGTGGCGGCCAACCCAGCATCCGCTGGGACGCCAATTT |
| | AATGACATGGTGTGAAGACCTGCATGTGCTTGATTGTGAGTCCTCCGGCCC |
| | CTGAATGCGGCTAACCCTAACCCCGGAGCCTTGCAGCACAATCCAGTGTTG |
| | TTAAGGTCGTAATGAGCAATTCTGGGATGGGACCGACTACTTTGGGTGTCC |
| | GTGTTTCTTATTTTTCTTGAATTTTTCTTATGGTCACAGCATATATACATTAT |
| | ATACTGTGATC |
| 4004 | TTAAAATAGCCTCAGGGTTGTTCCCACCCTGAGGGCCCACGTGGTGTAGTA |
| | CTCTGGTATTACGGTACCTTTGTACGCCTATTTTATACCCCCTTCCCCAAGT |
| | AATTTAGAAGCAAGCACAAACCAGTTCAGTAGTAAGCAGTACAATCCAGTA |
| | CTGTAATGAACAAGTACTTCTGTTACCCCGGAAGGGTCTATCGGTAAGCTG |
| | TACCCACGGCTGAAGAATGACCTACCGTTAACCGGCTACCTACTTCGAGAA |
| | GCCTAGTAATGCCGTTGAAGTTTTATTGACGTTACGCTCAGCACACTACCCC |
| | GTGTGTAGTTTTGGCTGATGAGTCACGGCACTCCCCACGGGCGACCGTGGC |
| | CGTGGCTGCGTTGGCGGCCAACCAAGGAGTGCAAGCTCCTTGGACGTCATA |
| | TTACAGACATGGTGTGAAGAGCCTATTGAGCTAGGTGGTAGTCCTCCGGCC |
| | CCTGAATGCGGCTAATCCTAACTCCGGAGCATATCGGTGCGAACCAGCACT |
| | TGGTGTGTTGTAATACGTAAGTCTGGAGCGGAACCGACTACTTTGGGTGTC |
| | CGTGTTTCCTGTTTTAACTTTTATGGCTGCTTATGGTGACAATTTAACATTGT |
| | TACCATATAGCTGTTGGGTTGGCCATCCGGATTTTGTTATAAAACCATTTCC |
| | TCGTGCCTTGACCTTTAACACATTTGTGAACTTCTTTAAATCCCTTTTATTAG |
| | TCCTTAAATACTAAGA |
| 4005 | TTCAAACAGCCTGGGGGTTGTACCCACCCCTGGGGCCCACGTGGCGCTAGT |
| | ACTCTGGTACGTTAGTACCTTTGTACGCCTGTTTTCCCCTCCCTTAAACAAA |
| | TTAAGATTACCACTACTGAGGGGAGTAGTCCGACTCCGCTCCGGTACTGCC |
| | GCACCAGTACTCCGGTACACTTAGTACCCTAGTACGGAGTAGATGGTATCC |
| | CCACCCCGCAACTTAGAAGCATGCAAACAAACCGACCAATAGGCGCACGA |
| | TATCCAGTCGTGTTTCGGTCAAGCACTTCTGTCTCCCCGGTCCGAAAGGATC |
| | GTTACCCGCCCGACCCACTACGAGAAGCCCAGTAACTGGCCAAGTGATTGC |
| | GAAGTTGCGCTCAGCCACAACCCCAGTGGTAGCTCTGGAAGATGGGGCTCG |
| | CGTCTCCCCCGTGGTGACACGGTCGCTTGCCCGCGTGTGCTTCCGGGTTCGG |
| | CCTACGCCGTTCACTTCAATGTCACGTAACCAGCCAAGAGCCTATTGTGCTG |
| | GGACGGTTTTCCTCCGGGGCCGTGAATGCTGCTAATCCCAACCTCCGAGCG |
| | TGTGCGCACAACCCAGTGTTGCTACGTCGTAATGCGTAAGTTGGAGGCGGA |
| | ACAGACTACTTTCGGTACCCCGTGTTTCCTTTAAATTTTATTCATTATTTTAT |
| | GGTGACAATTGCTGAGATCTGCGAATTAGCGACTCTGCCGTTGAATATTGC |
| | TCTGTACTATTTGGTTGCATTCCACAAAACCTCTGACATCCCCAGTACATAC |
| | ATTACTTTACTTGTTTACCTCAATCTAAAGCACAAGCTAGATAATACAAA |
| 4006 | TTTAAACAGCCTGGGGGTTGTTCCCACCCCTGGGGCCCACGTGGCGCTAGT |
| | ACTCTGGTACGCTAGTACCTTTGTACGCCTGTTTTTCCCCTCCCTTAAATAA |
| | ATCAAGGTTGCCACTACTGAGGGGAGTAGTCCGACTCCGCTCCAGCAATGC |
| | TGCACCAGTGCACTGGTACGCTAGTACCTTTTCACGGAGTAGATGGTATCC |
| | CTTACCCCGGAACCTAGAAGATTGCACACAAACCGACCAATAGGCGCACCG |
| | CATCCAGCCGTGCAGCGGTCAAGCACTTCTGTCTCCCCGGTCTGTAAAGAT |
| | CGTTATCCGCCCGACCCACTACGAAAAGCCTAGTAACTGGCCAAGTGAACG |
| | CGAAGTTGCGCTCCGCCACAACCCCAGTGGTAGCTCTGGAAGATGGGGCTC |
| | GCACCACCCCCGTGGTAACACGGTTGCCTGCCCGCGTGTGCTTCCGGGTTC |
| | GGTCTCGTGCCGTTCACTTCAACTTCACGCAACCAGCCAAGAGCCTATTGTG |
| | CTGGGACGGTTTTCCTCCGGGGCCGTGAATGCTGCTAATCCCAACCTCCGA |
| | GCGTGTGCGCACAATCCAGTGTTGCTACGTCGTAACGCGTAAGTTGGAGGC |
| | GGAACAGACTACTTTCGGTACCCCGTGTTTCCTCTCATTTTATTTAATATTTT |
| | ATGGTGACAATTGTTGAGATTTGCGCTCTTGCAACGTTGCCATTGAATATTG |
| | GCTTATACTATTTGGTTGCCTTTTACAAAACCTCTGATATACCCAGTTCTTA |
| | CATTGATCTGCTTGTTTTTCTCAATTTGAAGTATAGACTACAAATAGCAAA |
| 4007 | CCCCCCTCCCCCCCTTCCCTTCCCTTTGCAACGCAACAATTGTAAGTGCCCT |
| | CACCTGTCAATTGGGACCACCACTTTCAGTGACCCCATGCGAAGTGCTGAG |
| | AGAAAGGAAGCTTTCTTACCCTTCATTTGTGAACCCACTGGTCTAAGCCGCT |
| | TGGAATACGATGAGTGGAAAAGTTCATTCTTAATGGAGTGAAACATGCTTA |
| | AATTTCCAGCTCGTGCTGGTCTTTCCAGTACGGGGCGGCCCTGTCTGGCCGT |
| | AATTCTTCAGAGTGTCACGCCACACTTGTGGATCTCACGTGCCACATGACA |
| | GCGCTACAGCTGGAACTGGGTGCTTGGTGCCCATGGAGTAACAGCGAAAA |
| | GTGTTAGATCAAGCCTTGCTTGGGCTATGAGCCTGCGGAACAACAACTGGT |
| | AACAGTTGCCTCAGGGGCCGAAAGCCACGGTGTTAACAGCACCCTCATAGT |
| | TTGATCCACCTCAGGGTGGTGATGTTTAGCAGTTAGTAGTTGCCAATCTGTG |
| | TTCACTGAAATCTCGGCATACCGTGTAGTGTACAGGGGTGAAGGATGCCCA |
| | GAAGGTACCCGTAGGTAACCTTAAGAGACTATGGATCTGATCTGGGGCCTT |
| | GTCCGGAGTGCTTTACACACGGCTCAAGGTTAAAAAACGTCTAGCCCCACA |
| | GAGCCCGAGGGATTCGGGTTTTCCCTTTAAAAACCCGACTAGAGCTTATGG |
| | TGACAATTATTGCTGTTCAGACGAACAGTGTAATTGTTGTCTATTCACAGCA |
| | GTTCTATCAGAGCTTTTCCCACAACGGATCTTCTTGGCAAGCAAATACAGC |
| | AGGAGTCAAT |
| 4008 | CACTACGTTACGGTTCCCGCCCGGGACAACTGGTACCCCATTAGGCTACAA |
| | CATGGCTGAAAAGGGTATTGGGTCCCCCCGGATTGTGTCCGTTCGTAGTGT |
| | GTGTAACGTGGTTTACCATCTCCACTAACATTGGACTAAGCATTTCATCTTT |
| | CCTCCCCGATTGTGTACTCACTTGGCTAACGCTGGGTGGTCGCGGTTGGGTC |
| | CTTGATTTACTTTTTCTCGTCTAAGCATTCCGACTGTCCTCCCCGATTATGTG |
| | CTCATTCAGTTAACTGCTGGGTGGTCATGACTAACATCGAGGAACCTTCTGT |
| | CCACGCTTACTTTGAGCTCCGGTCGCTTGACGCTTGTAGGGCGATAGGGTTA |
| | TCTTCCTGACAACATCTTTATTCTACCTCCATAGGCTCTATCTATGGAGACG |
| | GAGTGTGGCACCCGTCCCTTCTTTGGGAGCTTCGGTAGTGACGCCCTTTGTC |
| | ACTCTCGCCAGCCGAGGCATGCCTGGTGCCAGGTAGCAAAGAAAGCATATG |
| | TTTAAGGACTTGACTGATTTAGCGCAAGAGTTTGTAGCGATGTCCATAGTGT |
| | CTGCGGATTCCCCACACGGCGACGTGTGCCGCGGAGGCCAAAAGCCACGGT |
| | GTTCACAGCACCCCTATGGATGCCCACAGACCCCAGTGGGCACTCTTGTTG |
| | CCGGACTTTCAGGAAATTAGGCATAGGCTCTTCTCAAACTCCTGGCATTGG |
| | ACTAGGTAAGAATGCCCCGGAGGTACCCCAGTACTCCTTCGGGAGTCTGGG |
| | ATCTGACCGGGGGCCCCACAAACATGCTTTACGTGTTTCGTGCGGTCAAAA |
| | ATTGTCTAACTAGTCCCAACCTTGAACAAGGGATTGTTCTTTCCTTTTTATT |
| | ACTGAGACTGGCCTATGGTGACAACAGAGATTGACTGTGAATACAGTTATT |
| | TTCTGGTGTTTATCATTTGGTTTTTCTCCGTGCTCTTTTACCTTTGTGGTATTT |
| | GTTCTTTAGATAGGCAAA |
| 4009 | CCCGGCCACCCCCTTTCGACGCGGGTACTGCGATAGTGCCACCCCAGTCTTT |
| | CCTACTCCCGACTCCCGACTCTAACCCAGGTTCCTTGGAACAGGAACACCA |
| | ATATACTCATCCCCTGGATGCTGACTAATCAGAGGAACGTCAGCATTTTCC |
| | GGCCCAGGCTAAGAGAAGTAGATAAGTTAGATTCCAAATTGATTTATCATC |
| | CCCTTGACGAATTCGCGTTGGAAATGCACCTCTCACTTGCCGCTCTTCACAC |
| | CCATTAACTTGATTCGGCCTCTGTGTTGAGCCCCTTGTTGAAGTGCTTCCCT |
| | CCATCGTGACGTGGTTGGAGATCTAAGTCAACCGACTCCGACGAAACTACC |
| | ATCATGCCTCCCCGATTATGTGATGCTTTCTGCCCTGCTGGGTGGAGCATCC |
| | TCGGGTTGAGAAAACCTTCTTCCTTTTTCCTTGGACCCCGGTCCCCCGGTCT |
| | AAGCCGCTTGGAATAAGACAGGGTTATCTTCACCTCTTCCTTCTTCTACTTC |
| | ATAGTGTTCTATACTATGAAAGGGTATGTGTCGCCCCTTCCTTCTTTGGAGA |
| | ACACGCGCGGCGGTCTTTCCGTCTCTCGAAAAGCGCGTGTGCGACATGCAG |
| | AGAACCGTGAAGAAAGCAGTTTGCGGACTAGCTTTAGTGCCCACAAGAAA |
| | ACAGCTGTAGCGACCACACAAAGGCAGCGGACCCCCCCTCCTGGCAACAG |
| | GAGCCTCTGCGGCCAAAAGCCACGTGGATAAGATCCACCTTTGTGTGCGGC |
| | ACAACCCCAGTGCCCTGGTTTCTTGGTGACACTTCAGTGAAAACGCAAATG |
| | GCGATCTGAAGCGCCTCTGTAGGAAAGCCAAGAATGTCCAGGAGGTACCCC |
| | TTCCCTCGGGAAGGGATCTGACCTGGAGACACATCACATGTGCTTTACACC |
| | TGTGCTTGTGTTTAAAAATTGTCACAGCTTTCCCAAACCAAGTGGTCTTGGT |
| | TTTCACTCTTTAAACTGATTTCACT |
| 4010 | CCCCCGGTTACCCCCTTTCGACGCGGGTACTGCGATAGTGCCACCCCAGTCT |
| | TTCCTACTCCCGACTCCCGACCCTAACCCAGGTTCCTCGGAACAGGAACAC |
| | AAATTTACTCATCCCCTGGATGCTGACTAATCAGAGGAACGTCAGCATTTTC |
| | CGGCCCAGGCTTAGAGAAGTAGATAAGTTAGAATCTAAATTGATATGACTT |
| | CCCCTTGACGAATTCACGTTGGAAATGCACCCCTCACTTGCCGCTCTTCACA |
| | CCCACTAATTGATTCGGCCTACTGTGTTGAGCCCCTTGTTGAAGTGCTTCCC |
| | TCCCTCGTGACGTGGTTGGAGAAATCTTGTCACCCGACTCCGACGAAACTA |
| | CCATCATGCCTCCCCGATTATGTGATGCTTTCTGCCCTGCTGGGTGGAGTAT |
| | CCTCGGGTTGAGAAATCCTTCTTCCTTTTACCTTGGACCTTGGTCCCCCGGT |
| | CTAAGCCGCTTGGAATAAGACAGGGTTATTTTCACCTCTTCTTCTTCTACTT |
| | CATGGTGCTCTATACCATGAAAGGGTATGTGTCGCCCCTTCCTTCTTGGAGA |
| | ACTCACGCGGCGGTCTTCCGTCTCTCAAAAAGCGCGAGTGCGACATGCAGA |
| | GTAACGCGAAGAAAGCAGTTCCTGGCCTAGCTCTAGTGCCCACAAGAAAAC |
| | GGCTGTAGCGACCACACAAAGGCAGCGGAACTCCCCTCCTGGTAACAGGA |
| | GCCTCTGCGGCCAAAAGCCACGTGGATTAGATCCACCTTTGTGTGCGGTGC |
| | AACCCCAGCACCCCGGTTTCTTGTTGACACTCTAGTGAATCCTTGAATGGCA |
| | ATCTCAAGCGCCTCTGTAGGAAAGCCAAGAATGTCCAGGAGGTACCCCTTC |
| | CTCGCGGAAGGGATCTGACCTGGAGACACATCACACGTGCTTTACACTTGT |
| | GCTTGTGTTTAAAAATTGTCACAGCTTTCCCAAACCAAGTGGTCTTGGTTTT |
| | CCTTTTTTATCCTACTGTCAAT |
| 4011 | TCCTCACCCATGCTTTTCCTACCCCCACCACGCCCGCATGTTTACTGCTTTCC |
| | TTGATGCTGCCCGTGACTACTTCCATGACCTCCCCAACCCAAACCTCAAACG |
| | CCTTAAGCTATCATGGGCTCTCTATCACCAATCCCCTTCCTTCCCACCCAGA |
| | ACCCCCCCCTCCCTCCCCTACAACGTGTATGAGCAAGACGAGTTTGACAAG |
| | CTCGCCGAAGCCATGCTCACCCACTCTCCCTTCCCCACATCCCATTACCTCT |
| | TCCACCCCCTCCCGTCCAACGCCCGCACCATCGTCGAGCGTGAGGCTGACT |
| | GGGATGGCTGTGATCTAGAAAAGAAATGGCTCGACCTCGTCATAGAGGAC |
| | GATGCCAAGTTCCTTCTGGAGAACGGCTCTCTCCCGTTTGGCTCCACCCTTG |
| | CC |
| 4012 | TTCTCGCCTGAGTCAACAAAGCGAGAAACCTGCCCCTCCAGCGCCAGACGA |
| | GCGGCATAAAACTTGAACTTCTGGCATGCTCCACCACCCTTTTCCCCATTCC |
| | AACCCCCATTGCGCTCTCAAGGTCGCGCTTTTTCGAGACTAGCTCGGATTCA |
| | AAAGTTCCTGGCACCCTTTGCCCCTTCAGGCCCTTAAGGTAGGAACTGACCT |
| | TGTGCTGTGCCCTCGGTGCGGAAGTGCTACTGCGTAGCGATTGTAAGATCC |
| | CTTTGTGGTTCTGCCCTGGCAAGGTTATAGAGTACTGTGATCCGCTGCGGAT |
| | GCCATCCTGGTAACAGGACCCCCAGTGTGCGCAACAGTATGTTCACGGTCT |
| | TCCGTGTCCACCACATTCGGAACACTGCTCTCGTGAAACAGTGTGTGTCCA |
| | ATCCCTGCAATCAGTATCAACTACACCACCTAGGAATGCTAGGAAGGTACC |
| | CCGGTCCGCCGGGATCTGATCCTAGGCTAATTGTCTACGGTGGTGCTCCTTT |
| | TTATTTTCCACTTCAATTCATTGGTTACAACTGCTCGATCCCTGTGTTTGCTG |
| | CCCTTCTCTGCTCTCATCGCCATTCTCAAGTGTTCACACTGTCCAAGTTCCTT |
| | TGGTTGTTCGCTTCCACTTGCCACTGTCAACTCTTGTC |
| 4013 | TCACCCTCTTTTCCGGTGGTCCGGACCCAGACCACCGTTACTCCATTCAGCT |
| | TCTTCGGAACCTGTTTGGAGGAATTAAACGGGCACCCACCCACCTTCACCC |
| | CCTTTTCGTAACTAAGTGTGTGCCCAATCTCATGACTCCTGCTGACTTCACC |
| | GACCAGCAGTGTCCAAAACGCTAGGTGAATTTCCTTCCTCCCCCTCTGGGCT |
| | TCTGCCCAGCTCCCTCCCTCCAGCCTGACGTGCCACAGGCTGTGCAAAGAC |
| | CCCGCGAAAGCTGCCAAAAGTGGCAATTGTGGGTCCCCCCTTTGTAAAGGC |
| | GTCGAGTCTTTCTCCCTTAAGGCTAGACCCGTCAGTGAATTCTGTCGGGCAA |
| | CTAGTGACGCCACTGCATGCCTCCGACCTCGGCCGCGGAGTGCTGCCCCCC |
| | AAGTCGTGCCCCTGACTACAAGTTGTGCTGTCTGGCAAACATTGTCTGTGA |
| | GAATGTTCCGCTGTGGCTGCCAAGCCTGGTAACAGGCTGCCCCAGTGTGCG |
| | TAGTTCTCATCCAGACTTCGGTCTGGCAACTTGCTGTTAAGACACGGCGTAA |
| | GGGGCGTGTGCCAACGCCCTGGAACGAGTGTCCACTCTAATACCCCGAGGA |
| | ATGCTACGCAGGTACCCCTGGCTCCCCAGGGATCTGAGCGTAGGCTAATTG |
| | TCTAAGGGTATTTTCATTTCCCACTCTTTCTTTCTTGTTCATA |
| 4014 | TCTGTCCTCACCCCATCTTCCCTTCTTTCCTGCACCGTTACGCTTACTCGCAT |
| | GTGCATTGAGTGGTGCACGTGCTTGAACAAACAGCTACACTCACATGGGGG |
| | CGGGTTTTCCCGCCCTGCGGCCTCTCGCGAGGCCCACCCCTCCCCTTCCTCC |
| | CATAACTACAGTGCTTTGGTAGGTAAGCATCCTGATCCCCCGCGGAAGCTG |
| | CTCACGTGGCAACTGTGGGGACCCAGACAGGTTATCAAAGGCACCCGGTCT |
| | TTCCGCCTTCAGGAGTATCCCTGCTAGTGAATTCTAGTAGGGCTCTGCTTGG |
| | TGCCAACCTCCCCCAAATGCGCGCTGCGGGAGTGCTCTTCCCCAACTCACC |
| | CTAGTATCCTCTCATGTGTGTGCTTGGTCAGCATATCTGAGACGATGTTCCG |
| | CTGTCCCAGACCAGTCCAGTAATGGACGGGCCAGTGTGCGTAGTCGTCTTC |
| | CGGCTTGTCCGGCGCATGTTTGGTGAACCGGTGGGGTAAGGTTGGTGTGCC |
| | CAACGCCCGTACTTTGGTGATACCTCAAGACCACCCAGGAATGCCAGGGAG |
| | GTACCCCGCTTCACAGCGGGATCTGACCCTGGGCTAATTGTCTACGGTGGTT |
| | CTTCTTGCTTCCACTTCTTTCTACTGTTCATG |
| 4015 | ATTCTCGGGCTACGGCCCTGGAGCCACTCCGGCTCCTAAAGATTTAGAAGT |
| | TTGAGCACACCCGCCCACTAGGGCCCCCCATCCAGGGGGGCAACGGGCAA |
| | GCACTTCTGTTTCCCCGGTATGATCTGATAGGCTGTAACCACGGCTGAAAC |
| | AGAGATTATCGTTATCCGCTTCACTACTTCGAGAAGCCTAGTAATGATGGG |
| | TGAAATTGAATCCGTTGATCCGGTGTCTCCCCCACACCAGAAACTCATGAT |
| | GAGGGTTGCCATCCCGGCTACGGCGACGTAGCGGGCATCCCTGCGCTGGCA |
| | TGAGGCCTCTTAGGAGGACGGATGATATGGATCTTGTCGTGAAGAGCCTAT |
| | TGAGCTAGTGTCGACTCCTCCGCCCCCGTGAATGCGGCTAATCCTAACCCC |
| | GGAGCAGGTGGGTCCAATCCAGGGCCTGGCCTGTCGTAATGCGTAAGTCTG |
| | GGACGGAACCGACTACTTTCGGGAAGGCGTGTTTCCATTTGTTCATTATTTG |
| | TGTGTTTATGGTGACAACTCTGGGTAAACGTTCTATTGCGTTTATTGAGAGA |
| | TTCCCAACAATTGAACAAACGAGAACTACCTGTTTTATTAAATTTACACAG |
| | AGAAGAATTACA |
| 4016 | GTGGCCACGCCCGGGCCACCGATACTTCCCTTCACTCCTTCGGGACTGTTGG |
| | GGAGGAACACAACAGGGCTCCCCTGTTTTCCCATTCCTTCCCCCTTTTCCCA |
| | ACCCCAACCGCCGTATCTGGTGGCGGCAAGACACACGGGTCTTTCCCTCTA |
| | AAGCACAATTGTGTGTGTGTCCCAGGTCCTCCTGCGTACGGTGCGGGAGTG |
| | CTCCCACCCAACTGTTGTAAGCCTGTCCAACGCGTCGTCCTGGCAAGACTAT |
| | GACGTCGCATGTTCCGCTGCGGATGCCGACCGGGTAACCGGTTCCCCAGTG |
| | TGTGTAGTGCGATCTTCCAGGTCCTCCTGGTTGGCGTTGTCCAGAAACTGCT |
| | TCAGGTAAGTGGGGTGTGCCCAATCCCTACAAAGGTTGATTCTTTCACCAC |
| | CTTAGGAATGCTCCGGAGGTACCCCAGCAACAGCTGGGATCTGACCGGAGG |
| | CTAATTGTCTACGGGTGGTGTTTCCTTTTTCTTTTCACACAACTCTACTGCTG |
| | ACAACTCACTGACTATCCACTTGCTCTGTCACG |
| 4017 | TTTGCTCAGCGTAACTTCTCCGGGTTACGTGGAGACCAAAAGGCTACGGAG |
| | ACTCGGGCTACGGCCCTGGAGCACCTAGGTGCTCCTAAAGACGTTAGAAGT |
| | TGTACAAACTCGCCCAATAGGGCCCCCCAACCAGGGGGGTAGCGGGCAAG |
| | CACTTCTGTTTCCCCGGTATGATCTCATAGGCTGTACCCACGGCTGAAAGAG |
| | AGATTATCGTTACCCGCCTCACTACTTCGAGAAGCCCAGTAATGGTTCATG |
| | AAGTTGATCTCGTTGACCCGGTGTTTCCCCCACACCAGAAACCTGTGATGG |
| | GGGTGGTCATCCCGGTCATGGCGACATGACGGACCTCCCCGCGCCGGCACA |
| | GGGCCTCTTCGGAGGACGAGTGACATGGATTCAACCGTGAAGAGCCTATTG |
| | AGCTAGTGTTGATTCCTCCGCCCCCGTGAATGCGGCTAATCCCAACTCCGG |
| | AGCAGGCGGGCCCAAACCAGGGTCTGGCCTGTCGTAACGCGAAAGTCTGG |
| | AGCGGAACCGACTACTTTCGGGAAGGCGTGTTTCCTTTTGTTCCTTTTATCA |
| | AGTTTTATGGTGACAACTCCTGGTAGACGTTTTATTGCGTTTATTGAGAGAT |
| | TTCCAACAATTGAACAGACTAGAACCACTTGTTTTATCAAACCCTCACAGA |
| | ATAAGATAACA |

Mutations of IRES and accessory sequences are encompassed herein to increase or reduce IRES activities, for example, by truncating the 5′ and/or 3′ ends of an IRES, adding a spacer 5′ to an IRES, modifying the 6 nucleotides 5′ to the translation initiation site (Kozak sequence), modification of (e.g., mutations) alternative translation initiation sites, and creating chimeric/hybrid IRES sequences. In some embodiments, the IRES sequence in the polynucleotide disclosed herein comprises one or more of these modifications relative to a natural or native IRES.

ii. Synthetic TIEs: Aptamer Complexes, Modified Nucleotides, IRES Variants & Other Engineered TIEs

In certain embodiments, a TIE provided herein is a synthetic TIE. In some embodiments, a synthetic TIE comprises aptamer complex, synthetic IRES, or other engineered TIE capable of initiating translation of a linear RNA or circular RNA polynucleotide.

In some embodiments, one or more aptamer sequences are capable of binding to a component of a eukaryotic initiation factor to either enhance or initiate translation. In some embodiments, an aptamer may be used to enhance translation in vivo and in vitro by promoting specific eukaryotic initiation factors (eIF) (e.g., certain aptamers disclosed in International Pat. Appl. No. PCT/EP2018/078794 are capable of binding to eukaryotic initiation factor 4F (eIF4F)). In some embodiments, an aptamer or a complex of aptamers may be capable of binding to EIF4G, EIF4E, EIF4A, EIF4B, EIF3, EIF2, EIF5, EIF1, EIF1A, 40S ribosome, PCBP1 (polyC binding protein), PCBP2, PCBP3, PCBP4, PABP1 (polyA binding protein), PTB, Argonaute protein family, HNRNPK (heterogeneous nuclear ribonucleoprotein K), or La protein.

c. Stop Codon or Stop Cassette

In various embodiments, the intervening region and/or core functional element comprises a stop codon or stop cassette. In some embodiments, the sequence is located downstream to a TIE and coding element. In some embodiments, the sequence is located downstream to a coding element and upstream to a TIE. In some embodiments, the intervening region comprises a stop codon. In one embodiment, the intervening region comprises a stop cassette. In some embodiments, the stop cassette comprises at least 2 stop codons. In some embodiments, the stop cassette comprises at least 2 frames of stop codons. In the same embodiment, the frames of the stop codons in a stop cassette each comprise 1, 2 or more stop codons. In some embodiments, the stop cassette comprises a LoxP or a RoxStopRox, or frt-flanked stop cassette. In the same embodiment, the stop cassette comprises a lox-stop-lox stop cassette.

### D. Additional Elements

In various embodiments, a provided polynucleotide (e.g., a DNA template, a linear precursor RNA polynucleotide, or a circular RNA polynucleotide) further comprises one or more elements for enhancing circularization, translation, or both. In certain embodiments, these elements are located with specificity between or within the intron elements, exon elements, or intervening region of the polynucleotide.

As an example, but not intended to be limiting, a polynucleotide, a precursor RNA polynucleotide, or circular RNA can comprise an IRES transacting factor region, a miRNA binding site, a restriction site, an RNA editing region, a structural or sequence element, a granule site, a zip code element, and/or an RNA trafficking element or another specialized sequence as found in the art that enhances promotes circularization and/or translation of the protein encoded within the circular RNA polynucleotide.

In some embodiments, the polynucleotide, precursor RNA polynucleotide, or circular RNA comprises an IRES transacting factor (ITAF) region. In some embodiments, the IRES transacting factor region modulates the initiation of translation through binding to PC-P1-PCBP4 (polyC binding protein), PABP1 (polyA binding protein), PTB (polyprimidine tract binding), Argonaute protein family, HNRNPK (Heterogeneous nuclear ribonucleoprotein K protein), or La protein. In some embodiments, the IRES transacting factor region comprises a polyA, polyC, polyAC, or polyprimidine track. In some embodiments, the ITAF region is located within the intervening region or core functional element. In some embodiments, the ITAF region is located within the TIE.

In certain embodiments, the polynucleotide, precursor RNA polynucleotide, or circular RNA comprises a lncRNA, miRNA, or a miRNA sponge. In certain embodiments, at least one miRNA binding site is included. In some embodiments the miRNA binding site is located within the 5′ intron element, 5′ exon element, intervening region or core functional element, 3′ exon element, and/or 3′ intron element. In some embodiments, the miRNA binding site is located within the spacer within the intron element or exon element. In certain embodiments, the miRNA binding site comprises the entire spacer regions. In some embodiments, the 5′ intron element and 3′ intron elements each comprise identical miRNA binding sites. In another embodiment, the miRNA binding site of the 5′ intron element comprises a different, in length or nucleotides, miRNA binding site than the 3′ intron element. In one embodiment, the 5′ exon element and 3′ exon element comprise identical miRNA binding sites. In other embodiments, the 5′ exon element and 3′ exon element comprise different, in length or nucleotides, miRNA binding sites. In some embodiments, the miRNA binding sites are located adjacent to each other within the circular RNA construct, linear RNA polynucleotide precursor, and/or DNA template. In certain embodiments, the first nucleotide of one of the miRNA binding sites follows the first nucleotide last nucleotide of the second miRNA binding site. In some embodiments, the miRNA binding site is located within a translation initiation element (TIE) of an intervening region or core functional element. In one embodiment, the miRNA binding site is located before, trailing or within an internal ribosome entry site (IRES). In another embodiment, the miRNA binding site is located before, trailing, or within an aptamer complex.

Incorporation of miRNA sequences can permit tissue-specific expression of a coding sequence within an intervening region or core functional element. For example, in a circular RNA intended to express a protein in immune cells, miRNA binding sequences resulting in expression suppression in tissues such as the liver or kidney may be desired. Such miRNA binding sequences may be selected based on the cell or tissue expression of miRNAs. The unique sequences defined by the miRNA nomenclature are widely known and accessible to those working in the microRNA field. For example, they can be found in the miRDB public database. As a non-limiting example, one or more miR-122 target sites can be inserted in the circular RNA.

In some embodiments, the miR-122 site can comprise the following sequence:

(SEQ ID NO: 4018)
CAAACACCATTGTCACACTCCAA.

### E. Circular RNA

Also provided herein are circular RNAs, in some instances produced by the precursor RNA polynucleotides described herein.

In some embodiments, provided herein is a circular RNA polynucleotide comprising, in the following order, a 3′ self-spliced exon segment, an intervening region, and a 5′ self-spliced exon segment. In some embodiments, provided herein is a circular RNA polynucleotide comprising, in the following order, a 3′ self-spliced exon segment, a coding sequence, and a 5′ self-spliced exon segment. In some embodiments, provided herein is a circular RNA polynucleotide comprising, in the following order, a 3′ self-spliced exon segment, a translation initiation element (TIE), a coding sequence, and a 5′ self-spliced exon segment. In some embodiments, provided herein is a circular RNA polynucleotide comprising, in the following order, a 3′ self-spliced exon segment, a translation initiation element (TIE), a coding sequence with which the TIE is not naturally associated, and a 5′ self-spliced exon segment.

In some embodiments, provided herein is a circular RNA polynucleotide comprising: i) a 5′ combined accessory element; ii) an intervening region; and iii) a 3′ combined accessory element, where the intervening region is between the 5′ combined accessory element and the 3′ combined accessory element.

In some embodiments, the 5′ combined accessory element comprises a 3′ self-spliced exon segment. In some embodiments, the 3′ self-spliced exon segment comprises an exon segment or fragment thereof. In some embodiments, the 3′ self-spliced exon segment comprises a 3′ nucleotide of a 3′ splice site dinucleotide. In some embodiments, the 3′ self-spliced exon segment comprises an exon segment and a 3′ nucleotide of a 3′ splice site dinucleotide. In some embodiments, the exon segment comprises a natural exon sequence or non-naturally occurring sequence. In some embodiments, the 3′ splice site dinucleotides are distinct from the natural splice site dinucleotide(s) associated with a natural Group I or Group II intron sequence.

In some embodiments, the 3′ self-spliced exon segment comprises a sequence having a percent sequence identity of about 70%, 75%, 80%, 85%, 90%, 91%, 92%, 93%, 94%, 95%, 96%, 97%, 98%, 99% or more to a sequence selected from SEQ ID NOs: 2990-3668, 25573, and 25574. In some embodiments, the 3′ self-spliced exon segment is selected from an exon segment disclosed herein, e.g., in Table A or Table B, or SEQ ID NOs: 2990-3668, 25573, and 25574. See, e.g., supra. In some embodiments, the self-spliced exon segment is, e.g., 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides. In some embodiments, the circular RNA comprises a self-spliced exon segment that is 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides from the exonic sequences of Table A or is e.g., 5 nucleotides, 6 nucleotides, 7 nucleotides, 8 nucleotides, 9 nucleotides, or 10 nucleotides from the exonic sequences of Table B. See also SEQ ID NOs: 2990-3668, 25573, and 25574.

In some embodiments, the 3′ combined accessory element comprises a 5′ self-spliced exon segment. In some embodiments, the 5′ self-spliced exon segment comprises an exon segment or fragment thereof. In some embodiments, the 5′ self-spliced exon segment comprises a 5′ nucleotide of a 5′ splice site dinucleotide. In some embodiments, the 5′ self-spliced exon segment comprises an exon segment and a 5′ nucleotide of a 5′ splice site dinucleotide. In some embodiments, the exon segment comprises a natural exon sequence or non-naturally occurring sequence. In some embodiments, the 5′ splice site dinucleotides are distinct from the natural splice site dinucleotide(s) associated with a natural Group I or Group II intron sequence.

In some embodiments, the 5′ self-spliced exon segment comprises a sequence having a percent sequence identity of about 70%, 75%, 80%, 85%, 90%, 91%, 92%, 93%, 94%, 95%, 96%, 97%, 98%, 99% or more to a sequence selected from SEQ ID NOs: 2990-3668, 25573, and 25574. In some embodiments, the 5′ self-spliced exon segment is selected from an exon segment disclosed herein, e.g., in Table A or Table B, or SEQ ID NOs: 2990-3668, 25573, and 25574. See, e.g., supra. In some embodiments, the self-spliced exon segment is e.g., 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides. In some embodiments, the circular RNA comprises a self-spliced exon segment that is 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides from the exonic sequences of Table A or is e.g., 5 nucleotides, 6 nucleotides, 7 nucleotides, 8 nucleotides, 9 nucleotides, or 10 nucleotides from the exonic sequences of Table B. See also SEQ ID NOs: 2990-3668, 25573, and 25574.

In some embodiments, as set forth herein, the intervening region comprises a noncoding region or a coding region. In some embodiments, the intervening region comprises at least one translation initiation element (TIE). In some embodiments, the TIE comprises a viral or eukaryotic internal ribosome entry site (IRES) and a noncoding or coding region. In some embodiments, the IRES comprises a sequence selected from the sequences in Table 1 or a fragment thereof or a sequence selected from SEQ ID NOS: 1-2989 and 4045-25570. See, e.g., supra. In some embodiments the TIE comprises a coding sequence with which the TIE is not naturally associated.

In some embodiments, the intervening region comprises an untranslated region (UTR). In some embodiments, the UTR comprises one or more noncoding elements. In some embodiments, the one or more noncoding elements are selected from, e.g., a natural 3′ Untranslated Region (UTR), a natural 5′ Untranslated Region (UTR), a synthetic spacer sequence, an aptamer, and lncRNA, miRNA, and a miRNA sponge. In some embodiments, the noncoding element is or comprises the TIE.

In some embodiments, the intervening region comprises a comprises a coding element or coding region. In some embodiments, the coding element comprises a sequence encoding at least one therapeutic protein. In some embodiments, the coding element encodes two or more polypeptides. In some embodiments, the coding element or coding region comprises a sequence encoding, for example, a therapeutic protein, cytokine, immune checkpoint inhibitor, an agonist, a chimeric antigen receptor, an inhibitory receptor agonist or inhibitory receptor, an inhibitory receptor antagonist, one or more TCR chains, a secreted T cell or immune cell engager, a transcription factor, an immunosuppressive enzyme, or a TvHd, as set forth in detail herein. In some embodiments, the coding element or coding region comprises one or more expression sequences or portions thereof, e.g., Table 2, infra.

In some embodiments, provided herein are circular RNA polynucleotides comprising, in the following order, i) a 5′ combined accessory element comprising a 3′ self-spliced exon segment; ii) an intervening region; and iii) a 3′ combined accessory element comprising a 5′ self-spliced exon segment. In some embodiments, the 3′ self-spliced exon segment and/or the 5′ self-spliced exon segment is selected from an exon segment disclosed herein, e.g., in Table A or Table B, or SEQ ID NOs: 2990-3668, 25573, and 25574.

In some embodiments, provided herein are circular RNA polynucleotides comprising, in the following order, i) a 5′ combined accessory element comprising a 3′ self-spliced exon segment, wherein the 3′ self-spliced exon segment comprises an exon segment; ii) an intervening region; and iii) a 3′ combined accessory element comprising a 5′ self-spliced exon segment, wherein the 5′ self-spliced exon segment comprises an exon segment. In some embodiments, the 3′ self-spliced exon segment and/or the 5′ self-spliced exon segment is selected from an exon segment disclosed herein, e.g., in Table A or Table B, or SEQ ID NOs: 2990-3668, 25573, and 25574.

In some embodiments, provided herein are circular RNA polynucleotides comprising, in the following order, i) a 5′ combined accessory element comprising a 3′ self-spliced exon segment, wherein the 3′ self-spliced exon segment comprises an exon segment and a 3′ nucleotide of a 3′ splice site dinucleotide; ii) an intervening region; and iii) a 3′ combined accessory element comprising a 5′ self-spliced exon segment, wherein the 5′ self-spliced exon segment comprises an exon segment and a 5′ nucleotide of a 5′ splice site dinucleotide. In some embodiments, the 3′ self-spliced exon segment and/or the 5′ self-spliced exon segment is selected from an exon segment disclosed herein, e.g., in Table A or Table B, or SEQ ID NOs: 2990-3668, 25573, and 25574.

A circular RNA polynucleotide comprising, in the following order, a 3′ self-spliced exon segment, an intervening region, and a 5′ self-spliced exon segment, wherein at least one of the 3′ or 5′ self-spliced exon segments is selected from an exon segment comprising a sequence selected from SEQ ID NOs: 2990-3668, 25573, and 25574.

As a non-limiting example, a circular RNA polynucleotide comprises the following elements operably connected and arranged in the following sequence:
(a) a 3′ exon segment comprising a Group I or Group II exon 3′ nucleotide of a 3′ splice site dinucleotide;
(b) an intervening region; and
(c) a 5′ exon segment comprising a Group I or Group II exon 5′ nucleotide of a 5′ splice site dinucleotide.

As set forth in detail herein, in some embodiments, a circular RNA polynucleotide comprises a retained portion of a monotron element. See, e.g., supra. In some embodiments, a circular RNA polynucleotide comprises: a 5′ internal spacer, a 5′ internal duplex, at least a portion of a terminal element (or sequence or segment), at least a portion of a monotron element (or sequence or segment), a 3′ internal duplex, a 3′ internal spacer, a coding or noncoding region, and an intervening region. In some embodiments, the circular RNA polynucleotide comprises a coding region and the intervening region comprises an IRES. In some embodiments, the monotron element present in the precursor RNA polynucleotide, of which a portion is retained in the circular RNA polynucleotide, comprises a polynucleotide sequence that has a percent sequence identity of about 70%, 75%, 80%, 85%, 90%, 91%, 92%, 93%, 94%, 95%, 96%, 97%, 98%, 99% or more to a sequence selected from SEQ ID NOs: 2990-3187, 25573, and 25574.

In some embodiments, the circular RNA polynucleotide comprises the following elements operably connected and arranged in the following sequence:
(a) a 5′ internal spacer,
(b) a 5′ internal duplex,
(c) at least a portion of a terminal element,
(d) at least a portion of a monotron element,
(e) a 3′ internal duplex,
(f) a 3′ internal spacer, and
(g) an intervening region, optionally comprising a coding region, and IRES.

In some embodiments, the circular RNA polynucleotide comprises the following elements operably connected and arranged in the following sequence:
(a) a 5′ internal spacer,
(b) a 5′ internal duplex,
(c) at least a portion of a monotron element,
(d) at least a portion of a terminal element,
(e) a 3′ internal duplex,
(f) a 3′ internal spacer, and
(g) an intervening region, optionally comprising a coding region, and IRES.

As a further non-limiting example, a circular RNA polynucleotide comprises the following elements operably connected and arranged in the following sequence:
(a) at least a portion of a terminal element,
(b) a 3′ exon segment comprising a 3′ nucleotide of a 3′ splice site dinucleotide,
(c) an intervening region,
(d) a 5′ exon segment comprising a 5′ nucleotide of a 5′ splice site dinucleotide, and
(e) at least a portion of a monotron element;
wherein the 5′ and/or 3′ splice site dinucleotides are distinct from the natural splice site dinucleotide(s) associated with a natural Group I or Group II intron sequence.

In some embodiments, element (d) comprises the first nucleotide of a 5′ Group I or Group II splice site dinucleotide and a natural exon sequence. In some embodiments, element (b) comprises the second nucleotide of a 3′ Group I or Group II exon splice site dinucleotide and a natural exon sequence.

In some embodiments, in the circular RNA polynucleotide, the 5′ exon element comprises the second nucleotide of a 3′ Group I or Group II exon splice site dinucleotide and a natural exon sequence. In some embodiments, the 3′ exon element fragment comprises the first nucleotide of a 5′ Group I or Group II splice site dinucleotide and a natural exon sequence. In some embodiments, the 5′ exon element comprises a 5′ internal duplex; and the 3′ exon element comprises a 3′ internal duplex. In some embodiments, the 5′ exon element comprises a 5′ internal spacer. In some embodiments, the 3′ exon element comprises a 3′ internal spacer.

In some embodiments, the circular RNA polynucleotide comprises a 5′ internal duplex and a 3′ internal duplex. See, e.g., supra.

In some embodiments, the circular RNA polynucleotide comprises a 5′ internal homology region and/or a 3′ internal homology region. See, e.g., supra.

In some embodiments, the circular RNA polynucleotide comprises internal spacers (IS) of different lengths, e.g., a 5′ internal spacer and/or a 3′ internal spacer. See, e.g., supra.

In some embodiments, the circular RNA polynucleotide retains portions of the precursor RNA polynucleotides, described elsewhere herein in detail. In some embodiments, portions of the precursor RNA polynucleotide are removed upon circularization. For example, in some embodiments, the circular RNA polynucleotide does not comprise a 5′ external spacer and/or a 3′ external spacer. In some embodiments, the circular RNA polynucleotide does not comprise a 5′ intron segment and/or 3′ intron segment. In some embodiments, the circular RNA polynucleotide does not comprise affinity tags. In some embodiments, the circular RNA polynucleotide does not retain a portion of a monotron element. In certain embodiments, the circular RNA polynucleotide does not retain a monotron element.

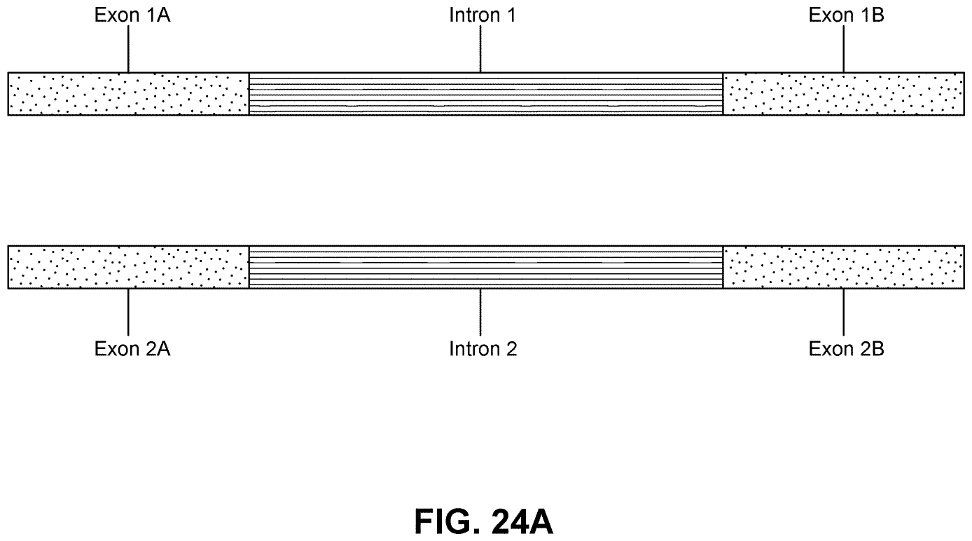

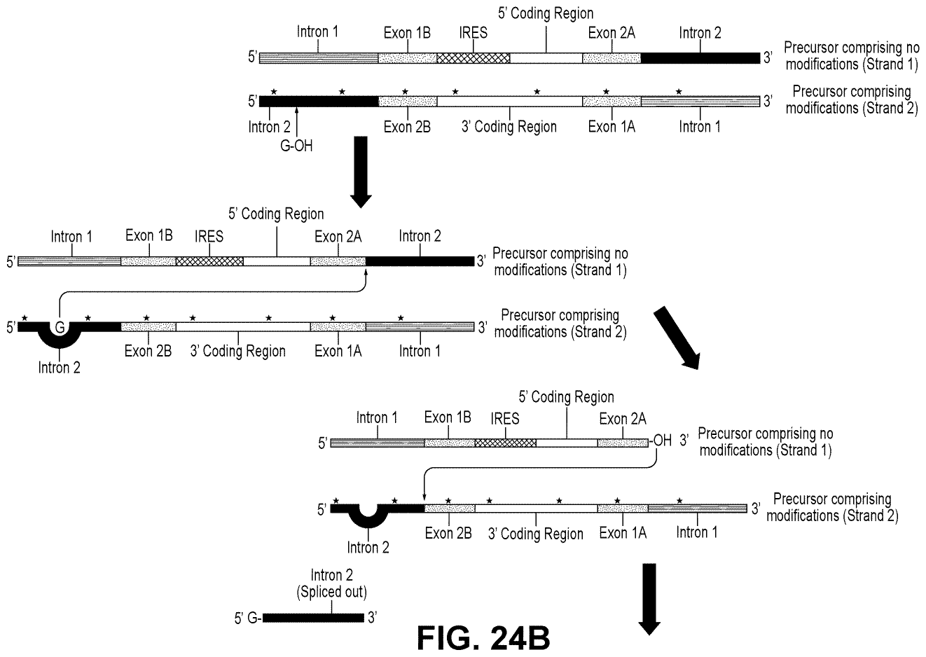

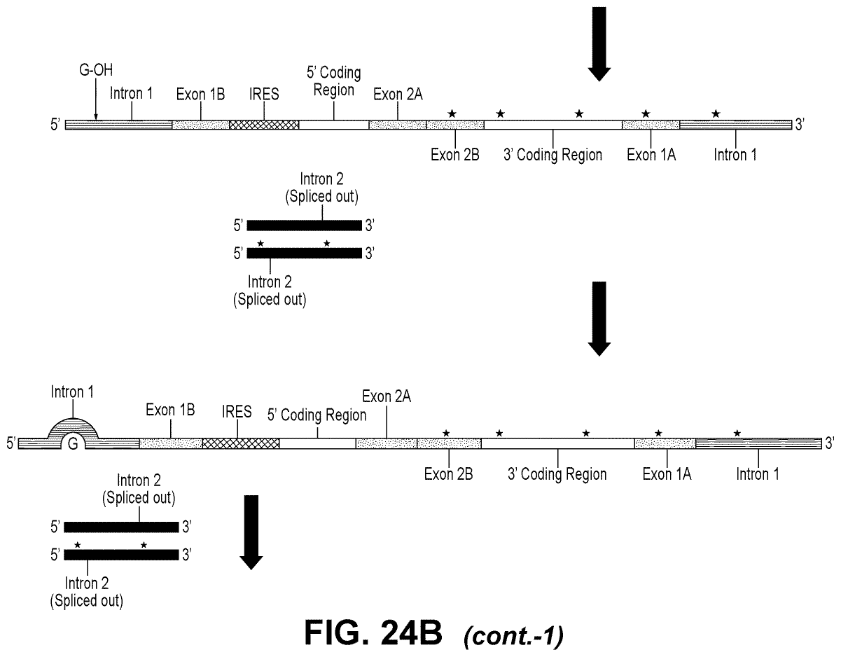

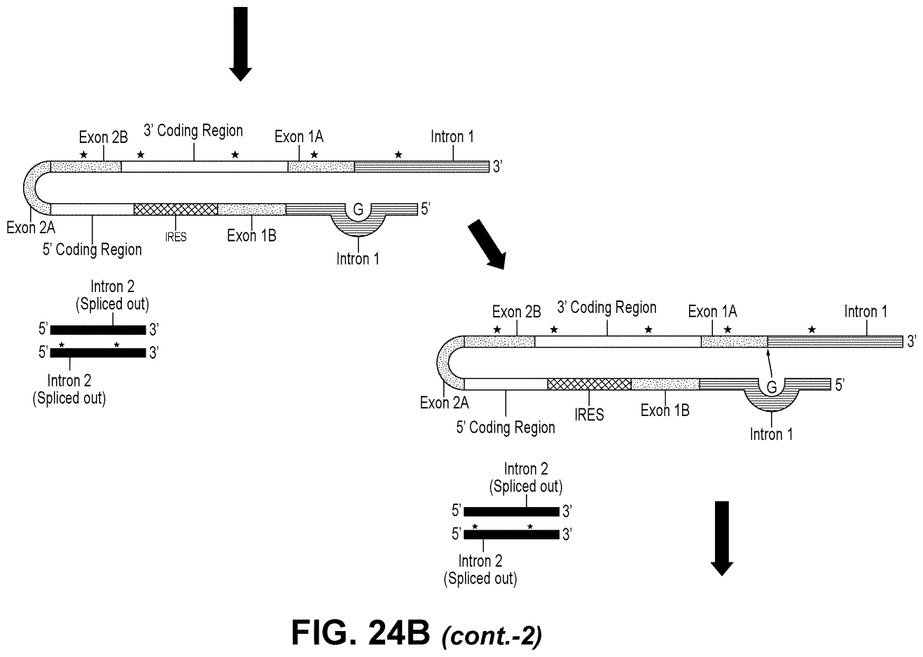

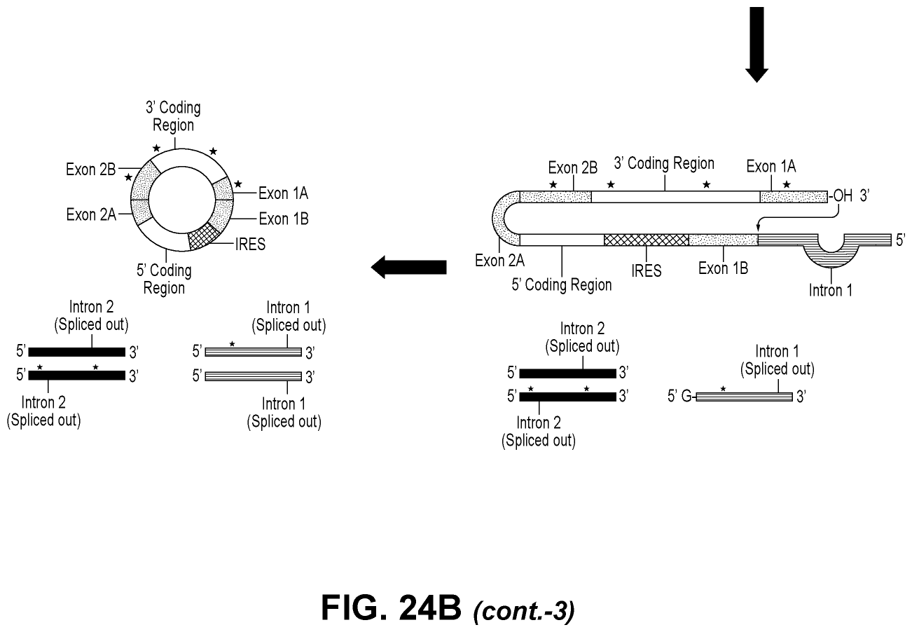

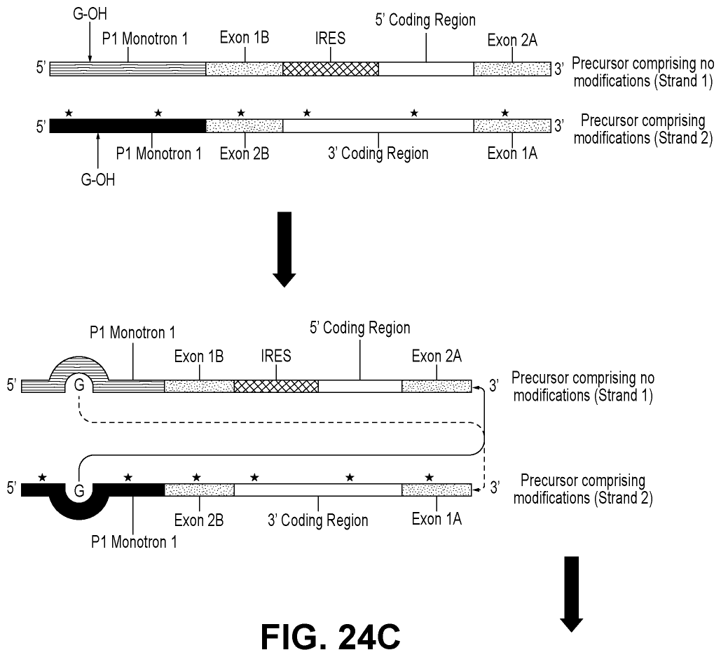

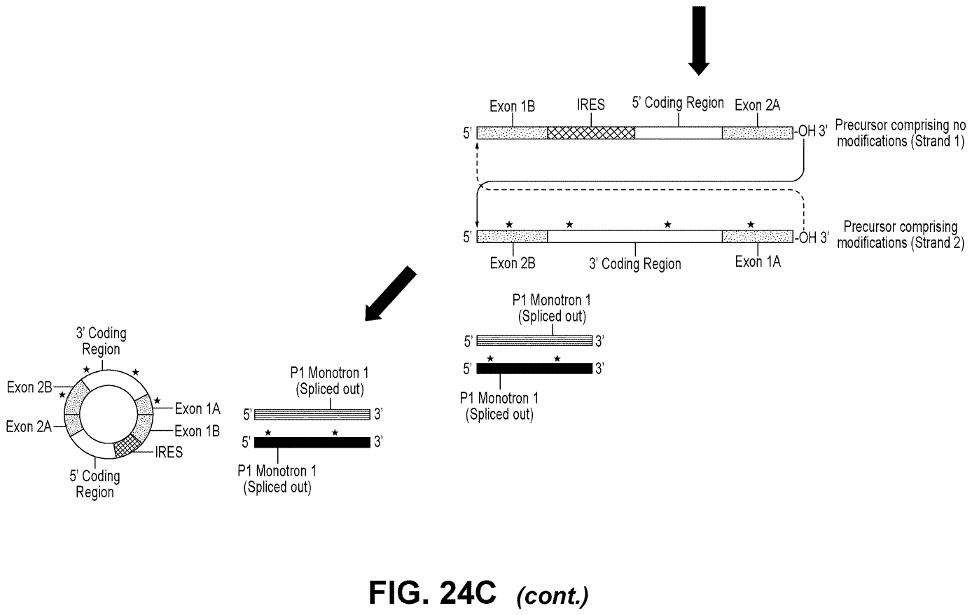

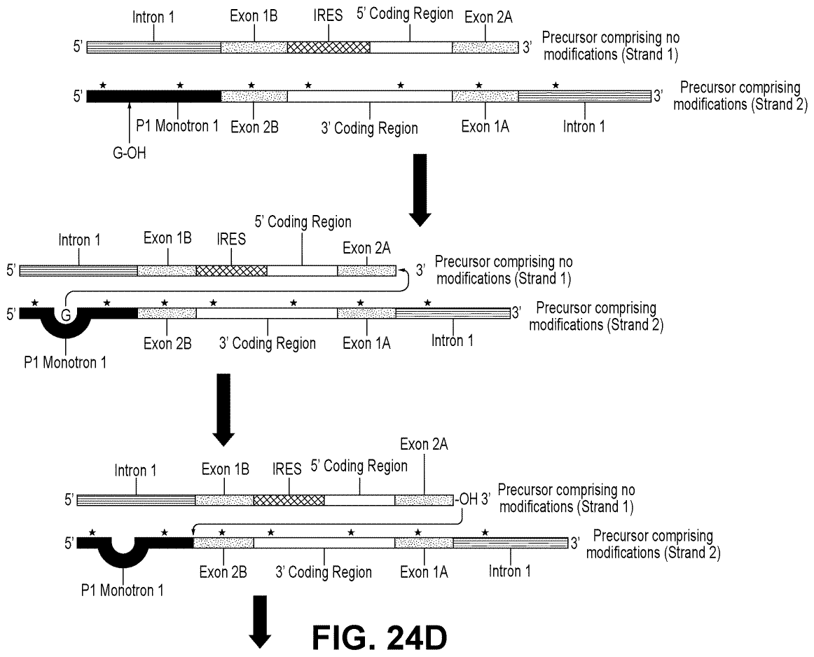

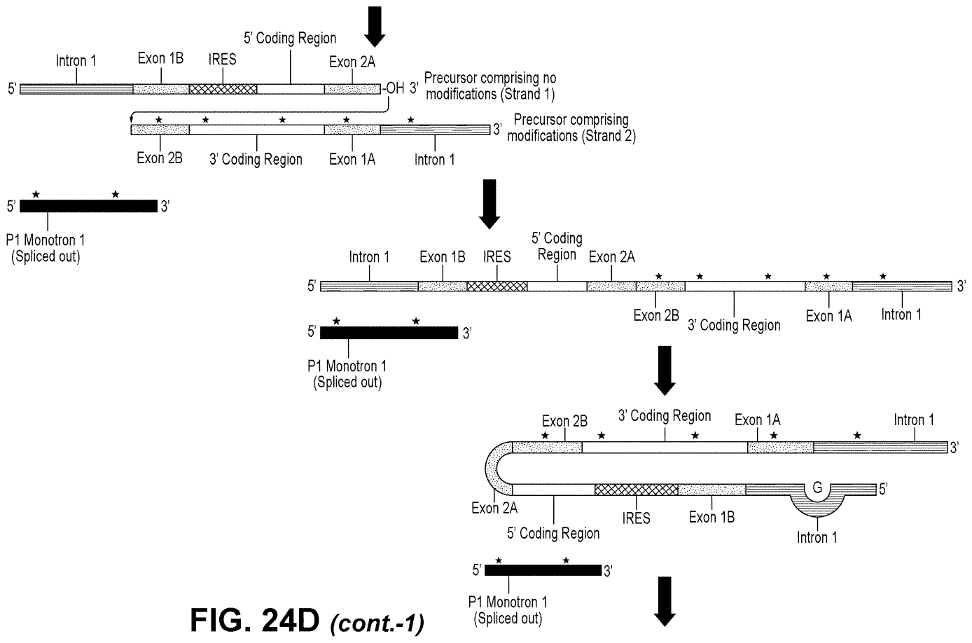

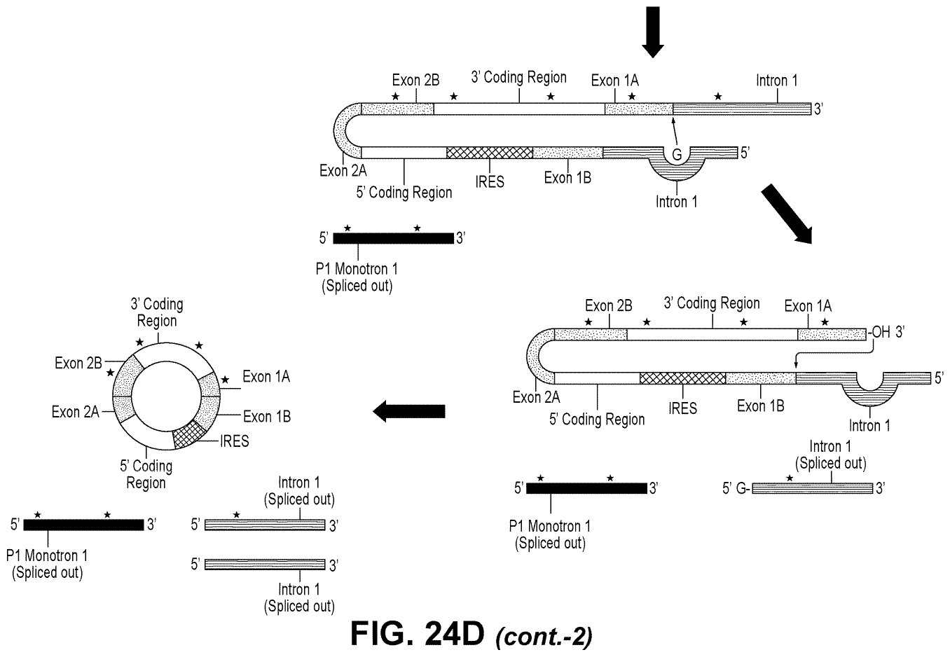

In some embodiments, and as described in more detail elsewhere herein, the circular RNA polynucleotide comprises modified nucleotides and/or modified nucleosides, namely comprising at least one modified A, C, G, or U/T nucleotide or nucleoside. Exemplary modifications are described in detail elsewhere herein. See, e.g., infra. In some embodiments, a circular RNA polynucleotide comprises modified nucleotides and/or modified nucleosides where between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides are modified. In some embodiments, portions of the polynucleotide comprise between 1% and 10% modification of the nucleotides or nucleosides. In some embodiments, portions of the circular RNA polynucleotide comprise less than 10% modification. In some embodiments, portions of the polynucleotide or the polynucleotide in its entirety comprise no nucleotide or nucleoside modifications. In some embodiments, a circular RNA polynucleotide may lack modifications, where the linear precursors used to produce the circular RNA polynucleotide contained modifications (e.g., in the introns). See, e.g., FIGS. 24B, 24C, and 24D. In some embodiments, incorporation of a nucleotide or nucleoside modification to a precursor RNA polynucleotide hinders or lowers the capacity of the circular RNA to circularize, splice, or express.

In some embodiments, the circular RNA polynucleotide is from about 50 nucleotides to about 15 kilobases in length.

In some embodiments, the circular RNA polynucleotide has an in vivo duration of therapeutic effect in a subject of at least about 10 hours. In some embodiments, the circular RNA polynucleotide has a functional half-life of at least about 10 hours. In some embodiments, the circular RNA polynucleotide has a duration of therapeutic effect in a cell greater than or equal to that of an equivalent linear RNA polynucleotide comprising the same expression sequence. In some embodiments, the circular RNA polynucleotide has a functional half-life in a cell greater than or equal to that of an equivalent linear RNA polynucleotide comprising the same expression sequence. In some embodiments, the circular RNA polynucleotide has an in vivo duration of therapeutic effect in a subject greater than that of an equivalent linear RNA polynucleotide having the same expression sequence. In some embodiments, the circular RNA polynucleotide has an in vivo functional half-life in a subject greater than that of an equivalent linear RNA polynucleotide having the same expression sequence.

In some embodiments, provided herein is a non-naturally occurring RNA polynucleotide comprising a translation initiation element (TIE), a coding sequence (e.g., with which the TIE is not naturally associated), and a means for self-splicing. See, e.g., Example 8, demonstrating that self-splicing efficiency and/or circularization efficiency is linked to the structures herein, e.g., at Examples 1, 2, 8. In some embodiments, provided herein is a non-naturally occurring RNA polynucleotide comprising a translation initiation element (TIE), a coding sequence (e.g., with which the TIE is not naturally associated), and a means for self-circularization. See, e.g., id In some embodiments, provided herein is provided herein is a non-naturally occurring RNA polynucleotide comprising a translation initiation element (TIE), a coding sequence (e.g., with which the TIE is not naturally associated), and an autocatalytic intron-exon means for self-splicing. See, e.g., id In some embodiments, provided herein is a non-naturally occurring RNA polynucleotide comprising a translation initiation element (TIE), a coding sequence (e.g., with which the TIE is not naturally associated), and an autocatalytic intron-exon means for self-circularization. See, e.g., id. In some embodiments, provided herein is a non-naturally occurring RNA polynucleotide comprising, in the following order, a 3′ exon segment means for self-splicing, a translation initiation element, a coding sequence, and a 5′ exon segment means for self-splicing. See, e.g., id. In some embodiments, provided herein is a non-naturally occurring RNA polynucleotide comprising, in the following order, a 3′ exon segment means for self-circularization, a translation initiation element, a coding sequence, and a 5′ exon segment means for self-circularization. See, e.g., id. In some embodiments, provided herein is a non-naturally occurring RNA polynucleotide comprising, in the following order, a 3′ exon segment, a translation initiation element, a coding sequence, and a 5′ exon segment, wherein the exon segments are means for self-splicing. See, e.g., id. In some embodiments, provided herein is a non-naturally occurring RNA polynucleotide comprising, in the following order, a 3′ exon segment, a translation initiation element, a coding sequence, and a 5′ exon segment, wherein the exon segments are means for self-circularization. See, e.g., id. In some embodiments, provided herein is a circular RNA polynucleotide comprising, in the following order, a 3′ exon segment means for self-circularization, a translation initiation element, a coding sequence, and a 5′ exon segment means for self-circularization. See, e.g., id. In some embodiments, provided herein is a circular RNA polynucleotide comprising, in the following order, a 3′ exon segment, a translation initiation element, a coding sequence, and a 5′ exon segment, wherein the exon segments are means for self-splicing. See, e.g., id.

### F. Modifications

In certain embodiments, a provided polynucleotide (e.g., a precursor RNA polynucleotide, a circular RNA polynucleotide, or a DNA template) comprises modified nucleotides and/or modified nucleosides, namely comprising at least one modified A, C, G, or U/T nucleotide or nucleoside. As exhibited by the exemplary nucleotide or nucleotide modification presented below, such modifications differ from mutations selected from insertions, deletions, addition, or subtraction of nucleotides, for example, the mutations in a permuted Group I and Group II intron segment.

In some embodiments, the polynucleotide is a precursor RNA polynucleotide and comprises at least one modified A, C, G, or U nucleotide or nucleoside. In some embodiments, the precursor RNA polynucleotide is linear. In some embodiments, the precursor RNA polynucleotide is capable of producing a circular RNA comprising at least one modified nucleotide or nucleoside after splicing. In some embodiments, the precursor RNA polynucleotide comprising one or more modified nucleotide or nucleoside is capable of circularizing when incubated in the presence of one or more guanosine nucleotides or nucleoside (e.g., GTP) and a divalent cation (e.g., Mg2+). In some embodiments, the polynucleotide is a circular RNA polynucleotide and comprises at least one modified A, C, G, or U nucleotide or nucleoside modifications.

In some embodiments, modified nucleotides or nucleosides occur throughout a precursor RNA polynucleotide. In some embodiments, the RNA polynucleotide comprises 5′ and 3′ combined accessory elements comprising one or more modified nucleotides. In some embodiments, the RNA polynucleotide comprises an intron element and/or exon element comprising one or more modified nucleotide or nucleoside.

In some embodiments, portions of the 3′ and/or 5′ intron and/or exon segments in a linear precursor RNA polynucleotide of the present disclosure contain modified nucleotides or nucleosides. In some embodiments, the secondary structures of at least the intron and/or exon segments are preserved. In some embodiments, the terminal element comprises at least one modified nucleotide or nucleoside. In some embodiments, the terminal element, intervening region, and/or monotron comprises at least one modified nucleotide or nucleoside. In certain embodiments, the RNA polynucleotide comprises a spacer comprising at least one modified nucleotide or nucleoside. In certain embodiments, the RNA polynucleotide comprises a duplex comprising at least one modified nucleotide or nucleoside. In certain embodiments, the RNA polynucleotide comprises an affinity sequence comprising at least one modified nucleotide or nucleoside. In certain embodiments, the RNA polynucleotide comprises a leading and/or lagging strand comprising at least one modified nucleotide or nucleoside. In some embodiments, the RNA polynucleotide comprises a coding or a noncoding element comprising at least one modified nucleotide or nucleoside. In some embodiments, the RNA polynucleotide comprises a translation initiation element (TIE) comprising at least one modified nucleotide or nucleoside. In certain embodiments, the polynucleotide comprises a stop codon and/or stop cassette comprising one or more modified nucleotide or nucleoside.

In some embodiments, a precursor RNA polynucleotide comprising at least one modified A, C, G, or U nucleotide or nucleoside comprises at least a portion of each of:
a. a 5′ combined accessory element, comprising:
   i. a 3′ intron segment,
   ii. a 3′ exon segment,
b. an intervening region comprising an internal ribosome entry site (IRES) and a noncoding or coding region,
c. a 3′ combined accessory element, comprising:
   i. a 5′ exon segment, and
   ii. a 5′ intron segment.

In some embodiments, a circular RNA comprising at least one modified A, C, G, or U nucleotide or nucleoside comprises at least a portion of each of:
a. a post-splicing 3′ exon segment,
b. optionally a 5′ internal homology region,
c. optionally a 5′ spacer,
d. an intervening region comprising an internal ribosome entry site (IRES) and a noncoding or coding region,
e. optionally a 3′ spacer,
f. optionally a 3′ internal homology region, and
g. a post-splicing 5′ exon segment.

In some embodiments, the modified nucleoside is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is T (pseudouridine). In another embodiment, the modified nucleoside is Um (2′-O-methyluridine). In other embodiments, the modified nucleoside is m¹A (1-methyladenosine); m²A (2-methyladenosine); Am (2′-O-methyladenosine); ms² m6A (2-methylthio-N⁶-methyladenosine); i⁶A (N⁶-isopentenyladenosine); ms²i6A (2-methylthio-N⁶ isopentenyladenosine); io⁶A (N⁶-(cis-hydroxyisopentenyl)adenosine); ms²io⁶A (2-methylthio-N⁶-(cis-hydroxyisopentenyl)adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms² hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); fVC (5-formylcytidine); msCm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m² 2G (N²,N²-dimethylguanosine); m²Gm (N²,2′-O-dimethylguanosine); m² ₂Gm (N²,N²,2′-O-trimethylguanosine); Gr(p) (2′-O-ribosylguanosine(phosphate)); yW (wybutosine); o₂yW (peroxywybutosine); oHyW (hydroxywybutosine); OhyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵S²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶ ₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶ ₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm ⁵U (5-taurinomethyluridine); τm⁵s²U (5-taurinomethyl-2-thiouridine)); imG-14 (4-demethylwyosine); imG2 (isowyosine); N1-methylpseudouridine; or ac⁶A (N⁶-acetyladenosine).

In some embodiments, the modified nucleoside may include a compound selected from the group of: 146yridine-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, N1-methylpseudouridine; and N2,N2-dimethyl-6-thio-guanosine.

In another embodiment, the modifications are independently selected from 5-methylcytosine, pseudouridine and 1-methylpseudouridine.

In some embodiments, the modified ribonucleosides include 5-methylcytidine, 5-methoxyuridine, 1-methyl-pseudouridine, N6-methyladenosine, and/or pseudouridine.

In some embodiments, the modified nucleoside is N1-methylpseudouridine.

In some embodiments, the modified nucleotide or nucleoside is selected from one or more of: 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine, 5-(2-amino)propyl uridine, 5-halocytidine, 5-halouridine, 4-acetylcytidine, 1-methyladenosine, 2-methyladenosine, 3-methyicytidine, 6-methyluridine, 2-methylguanosine, 7-methylguanosine, 2,2-dimethylguanosine, 5-methylaminoethyluridine, 5-methyloxyuridine, 7-deaza-adenosine, 6-azouridine, 6-azocytidine, 6-azothymidine, 5-methyl-2-thiouridine, 2-thiouridine, 4-thiouridine, 2-thiocytidine, dihydrouridine, pseudouridine, queuosine, archaeosine, naphthyl substituted naphthyl groups, an O- and N-alkylated purines and pyrimidines, N6-methyladenosine, 5-methylcarbonylmethyluridine, uridine 5-oxyacetic acid, pyridine-4-one, pyridine-2-one, aminophenol, 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides.

Additional modified nucleotides and nucleosides can be selected from clinically validated modified nucleotides described in the art. See, e.g., US20190345503A1 (m⁶A-modified circRNA); US20220288176A1 (m⁶A modification of circRNA); US20220251578A1 (at least one N6-methyladenosine (m⁶A)); WO2022271965A2 (N6-methyladenosine, 2-thiouridine, and 2′-O-methylcytidine), which are each incorporated by reference in their entireties.

In some embodiments, a first and second precursor polynucleotide are provided, where the first precursor RNA polynucleotide comprises a 3′ intron fragment of a first intron (Intron 1), a 5′ intron fragment of a second intron (Intron 2), a translation initiation element, a fragment of a sequence of interest (e.g., coding region), and two exon fragments that correspond with the intron fragments; and the second precursor comprises a 3′ intron fragment of the second intron (Intron 2) and a 5′ intron fragment of the first intron (Intron 1), a fragment of the sequence of interest of the first precursor, and exon fragments corresponding to those in the first precursor. In these embodiments, the first and second linear precursor RNA polynucleotides are capable of forming a circular RNA. In some embodiments, the first precursor comprises no nucleotide or nucleoside modifications and the second precursor comprises nucleotide or nucleoside modifications. In some embodiments, the first precursor comprises nucleotide or nucleoside modifications and the second precursor comprises no nucleotide or nucleoside modifications. In some embodiments, the first precursor and the second precursor comprise no nucleotide or nucleoside modifications. In some embodiments, the first precursor and the second precursor comprise nucleotide or nucleoside modifications.

Indeed, contrary to publications contending that, for example, “[i]ncorporation of m6A modification into circRNA does not affect splicing to form circRNA” (see, e.g., Chen et al., 2019, Mol Cell, N6-Methyladenosine Modification Controls Circular RNA Immunity), the disclosures herein demonstrate that the incorporation of certain nucleotide and/or nucleoside modifications to a precursor RNA polynucleotide can affect the circularization and/or splicing of the circular RNA. (See Kariko et al., 2005, Immunity, Suppression of RNA recognition by Toll-like receptors: the impact of nucleoside modification and the evolutionary origin of RNA; Kariko et al., 2005, Mol Ther, Incorporation of pseudouridine into mRNA yields superior nonimmunogenic vector with increased translational capacity and biological stability; Wesselhoeft et al., 2019, Mol Cell, RNA Circularization Diminishes Immunogenicity and Can Extend Translation Duration In Vivo; Chen et al., 2022, Nature Biotechnology, Engineering circular RNA for enhanced protein production). Modified nucleotide or nucleosides may exhibit different physical properties to their unmodified counterparts. In some embodiments, the presence of a modified nucleotide or nucleoside can affect the folding patterns and/or function of an accessory element, translation initiation element (TIE), and/or coding element within the circular RNA or linear precursor. Position and composition of a nucleotide or nucleoside modification in a polynucleotide are impacted by the nucleotide or nucleoside composition (i.e., A, C, G, or U nucleotide or nucleoside) of the accessory elements, TIE, or coding elements.

In some embodiments, in a provided polynucleotide (e.g., a precursor RNA polynucleotide, or a circular RNA polynucleotide, described in more detail elsewhere herein), between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides are unmodified. In some embodiments, a provided polynucleotide (e.g., a precursor RNA polynucleotide, or a circular RNA polynucleotide, described in more detail elsewhere herein) comprises modified nucleotides and/or modified nucleosides where between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides are modified. In some embodiments, between 1% and 10% of the nucleotides or nucleosides are modified.

In some embodiments, in portions of the polynucleotide (e.g., a precursor RNA polynucleotide, or a circular RNA polynucleotide, described in more detail elsewhere herein), between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides are modified. For example, in some embodiments, between 1% and 100%, 1% and 2%, 1% and 3%, 1% and 4%, 1% and 5%, 5% and 6%, 5% and 7%, 55% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides in the intervening region are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides in the IRES are modified. In some embodiments, between 1% and 100%, 1% and 2%, 1% and 3%, 1% and 4%, 1% and 5%, 5% and 6%, 5% and 7%, 55% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides in the noncoding or coding region are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides in the 5′ intron segment and/or 3′ intron segment are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides in the 5′ exon segment or post-splicing exon segment and/or 3′ exon segment or post-splicing exon segment are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides in the 5′ spacer and/or 3′ spacer are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides in the 5′ homology region and/or 3′ homology region are modified. In some embodiments, the secondary structures of at least the intron and/or exon segments are preserved. In some embodiments, the secondary structure of the coding or noncoding region is preserved. In some embodiments, the IRES is unmodified or substantially unmodified to preserve secondary structure needed to initiate translation.

In some embodiments, between 1% and 10% of the nucleotides or nucleosides are modified in a polynucleotide of the present disclosure (e.g., a precursor RNA polynucleotide, or a circular RNA polynucleotide, described in more detail elsewhere herein). In some embodiments, portions of the polynucleotide comprise between 1% and 10% modification of the nucleotides or nucleosides. For example, in some embodiments, between 1% and 10% of the nucleotides or nucleosides in the intervening region are modified. In some embodiments, between 11% and 10% of the nucleotides or nucleosides in the IRES are modified. In some embodiments, between 1% and 10% of the nucleotides or nucleosides in the noncoding or coding region are modified. In some embodiments, between 1% and 10% of the nucleotides or nucleosides in the 5′ intron segment and/or 3′ intron segment are modified. In some embodiments, between 11% and 10% of the nucleotides or nucleosides in the 5′ exon segment or post-splicing exon segment and/or 3′ exon segment or post-splicing exon segment are modified. In some embodiments, between 1% and 10% of the nucleotides or nucleosides in the 5′ spacer and/or 3′ spacer are modified. In some embodiments, between 1% and 10% of the nucleotides or nucleosides in the 5′ homology region and/or 3′ homology region are modified.

In some embodiments, the polynucleotides comprising modified nucleotides and/or modified nucleosides provide additional stability and resistance to immune activation. In some embodiments, polynucleotides comprising modified nucleotides and/or modified nucleosides maintain stability and resistance to immune activation as compared to a corresponding polynucleotide comprising no modified nucleotides and/or modified nucleosides.

In some embodiments, a precursor RNA polynucleotide with modified nucleotides and/or nucleosides improves circularization as compared to a corresponding linear precursor RNA polynucleotide comprising no nucleotide or nucleoside modifications or other appropriate control. In other embodiments, a precursor RNA polynucleotide with modified nucleotides and/or nucleosides maintains the same circularization as compared to a corresponding precursor RNA polynucleotide comprising no nucleotide or nucleoside modifications or other appropriate control. In some embodiments, the precursor polynucleotides comprising modified nucleotides and/or modified nucleosides maintain circularization at greater than 50%, greater than 60%, greater than 70%, greater than 80%, greater than 90%, or greater than 100% (i.e., improves circularization), as compared to a corresponding precursor polynucleotide comprising no nucleotide or nucleoside modifications. In some embodiments, the precursor polynucleotides maintain circularization at greater than 70% as compared to a corresponding precursor polynucleotide comprising no nucleotide or nucleoside modifications. In some embodiments, the precursor polynucleotides maintain circularization at greater than 80% as compared to a corresponding precursor polynucleotide comprising no nucleotide or nucleoside modifications. In some embodiments, the precursor polynucleotides maintain circularization at greater than 90% as compared to a corresponding precursor polynucleotide comprising no nucleotide or nucleoside modifications. In some embodiments, the precursor polynucleotides exhibit greater than 100% circularization (i.e., improved circularization) as compared to a corresponding precursor polynucleotide comprising no nucleotide or nucleoside modifications.

In some embodiments a circular RNA with modified nucleotides and/or nucleosides reduces immunogenicity and/or improves translation of the coding region as compared to a corresponding circular RNA comprising no nucleotide or nucleoside modifications. In other embodiments, a circular RNA polynucleotide with modified nucleotides and/or nucleosides maintains the same immunogenicity and/or translation of the coding region as compared to a corresponding circular RNA comprising no nucleotide or nucleoside modifications. For example, in some embodiments, the circular RNAs described herein comprising at least one modified A, C, G, or U nucleotide or nucleoside exhibit reduced immunogenicity, without losing circularization and/or translation. In some embodiments, the circular RNAs described herein exhibit immunogenicity that is reduced by about 10% to about 99%, for example reduced by about 10%, 1%, 20%, 25%, 30%, 35%, 40%, 45%, 50^{%}, 55%, 60%, 65%, 70%, 75%, 80%, 85%, 90%, 95%, or 99% as compared to a corresponding circular RNA comprising no nucleotide or nucleoside modifications. In some embodiments, the polynucleotides comprising modified nucleosides provide additional stability and resistance to immune activation.

In some embodiments, portions of the polynucleotide (e.g., a precursor RNA polynucleotide, or a circular RNA polynucleotide) or the polynucleotide in its entirety comprises no nucleotide or nucleoside modifications. In some embodiments, portions of the polynucleotide (e.g., a precursor RNA polynucleotide, or a circular RNA polynucleotide, described in more detail elsewhere herein) comprise less than 10% modification. For example, in some embodiments, the intervening region comprises no nucleotide or nucleoside modifications or less than 10% of the nucleotides or nucleosides therein are modified. In some embodiments, the IRES comprises no nucleotide or nucleoside modifications or less than 10% of the nucleotides or nucleosides therein are modified. In some embodiments, the noncoding or coding region comprises no nucleotide or nucleoside modifications or less than 10% of the nucleotides or nucleosides therein are modified. In some embodiments, the 5′ intron segment and/or 3′ intron segment comprises no nucleotide or nucleoside modifications or less than 10% of the nucleotides or nucleosides therein are modified. In some embodiments, the 5′ exon segment or post-splicing exon segment and/or 3′ exon segment or post-splicing exon segment comprises no nucleotide or nucleoside modifications or less than 10% of the nucleotides or nucleosides therein are modified. In some embodiments, the 5′ spacer and/or 3′ spacer comprises no nucleotide or nucleoside modifications or less than 10% of the nucleotides or nucleosides therein are modified. In some embodiments, the 5′ homology region, 3′ homology region comprises no nucleotide or nucleoside modifications or less than 10% of the nucleotides or nucleosides therein are modified.

In some embodiments herein, between 1% and 10% of the nucleotides or nucleosides are modified in a linear precursor RNA polynucleotide or circular RNA of the present disclosure. In some embodiments in a linear precursor RNA polynucleotide or circular RNA of the present disclosure, the intervening region comprises no nucleotide or nucleoside modifications or is less than 10% modified; the IRES comprises no nucleotide or nucleoside modifications or is less than 10% modified; the noncoding or coding region comprises no nucleotide or nucleoside modifications or is less than 10% modified; the 5′ intron segment and/or 3′ intron segment comprises no nucleotide or nucleoside modifications or is less than 10% modified; the 5′ exon segment or post-splicing exon segment and/or 3′ exon segment or post-splicing exon segment comprises no nucleotide or nucleoside modifications or is less than 10% modified; the 5′ spacer and/or 3′ spacer comprises no nucleotide or nucleoside modifications or is less than 10% modified; and/or the 5′ homology region, 3′ homology region comprises no nucleotide or nucleoside modifications or is less than 10% modified.

In some embodiments, modified nucleotides or nucleotides occur throughout a precursor RNA polynucleotide. In other embodiments, portions of the 3′ and/or 5′ intron and/or exon segments in a linear precursor RNA polynucleotide of the present disclosure contain modified nucleotides or nucleosides, but the remaining portions of the linear precursor do not comprise nucleotide or nucleoside modifications. In some embodiments, portions of the 3′ and/or 5′ intron and/or exon segments in a linear precursor RNA polynucleotide of the present disclosure contain modified nucleotides or nucleosides, but the remaining portions of the linear precursor comprise minimal nucleotide or nucleoside modifications. In some embodiments, portions of the 3′ and/or 5′ intron and/or exon segments in a linear precursor RNA polynucleotide of the present disclosure contain modified nucleotides or nucleosides, but the remaining portions of the linear precursor comprise less than 90%, 80%, 70%, 60%, 50%, 40%, 30%, 20%, or 10% modified nucleotides or nucleosides.

In some embodiments, where the circular RNA is produced from a linear precursor and where the linear precursor is modified at the 3′ and/or 5′ ends only, the circular RNA contains only the modified nucleotide or nucleosides that remain after circularization.

In certain embodiments, a circular RNA is prepared by providing modified nucleotides or nucleosides to precursor RNA comprising:
a. a 5′ combined accessory element, comprising:
   i. a 3′ intron segment,
   ii. a 3′ exon segment,
b. an intervening region comprising an internal ribosome entry site (IRES) and a noncoding or coding region,
c. a 3′ combined accessory element, comprising:
   i. a 5′ exon segment, and
   ii. a 5′ intron segment.

In certain embodiments, a circular RNA is prepared by providing a first and second linear precursor RNA polynucleotide, wherein the first and second linear precursor RNA polynucleotides are capable of forming a circular RNA. In some embodiments, either the first precursor or the second precursor but not both precursors comprises at least one modified A, C, G, or U nucleotide or nucleoside. In some embodiments, the first precursor comprises at least one modified A, C, G, or U nucleotide or nucleoside and the second precursor comprises no modified nucleotides or nucleosides. In some embodiments, the second precursor comprises least one modified A, C, G, or U nucleotide or nucleoside and the first precursor comprises no modified nucleotides or nucleosides.

In some embodiments, the first precursor comprises a 3′ intron fragment of a first intron (Intron 1), a 5′ intron fragment of a second intron (Intron 2), a translation initiation element, a fragment of a sequence of interest (e.g., coding region), and two exon fragments that correspond with the intron fragments. In some embodiments, the second precursor comprises a 3′ intron fragment of the second intron (Intron 2) and a 5′ intron fragment of the first intron (Intron 1), a fragment of the sequence of interest of the first precursor, and exon fragments corresponding to those in the first precursor.

In some embodiments, the TIE of the first precursor RNA polynucleotides comprises an IRES. In some embodiments, the first precursor RNA polynucleotide comprises a noncoding or coding region.

In some embodiments, the first and second precursor RNA polynucleotides further comprise spacers and/or homology arms.

In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the first linear precursor are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the first linear precursor are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the first linear precursor are unmodified.

In some embodiments, in portions of the first linear polynucleotide, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides are modified. For example, in some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the intervening region of the first linear polynucleotide are modified. In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the TIE (e.g., IRES) of the first linear polynucleotide are modified. In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the noncoding or coding region of the first linear polynucleotide are modified. In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the 5′ and/or 3′ intron fragment of the first linear polynucleotide are modified. In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the 5′ and/or 3′ exon fragment of the first linear polynucleotide are modified. In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the spacer of the first linear polynucleotide are modified. In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the internal and/or external homology region of the first linear polynucleotide are modified.

In some embodiments, in portions of the first linear polynucleotide, less than 10% of the nucleotides or nucleosides are modified. For example, in some embodiments, less than 10% of the nucleotides or nucleosides in the intervening region of the first linear polynucleotide are modified. In some embodiments, less than 10% of the nucleotides or nucleosides in the TIE (e.g., IRES) of the first linear polynucleotide are modified. In some embodiments, less than 10% of the nucleotides or nucleosides in the noncoding or coding region of the first linear polynucleotide are modified. In some embodiments, less than 10% of the nucleotides or nucleosides in the 5′ and/or 3′ intron fragment of the first linear polynucleotide are modified. In some embodiments, less than 10% of the nucleotides or nucleosides in the 5′ and/or 3′ exon fragment of the first linear polynucleotide are modified. In some embodiments, less than 10% of the nucleotides or nucleosides in the spacer of the first linear polynucleotide are modified. In some embodiments, less than 10% of the nucleotides or nucleosides in the internal and/or external homology region of the first linear polynucleotide are modified.

In some embodiments, the intervening region of the first linear polynucleotide comprises no nucleotide or nucleoside modifications. In some embodiments, the TIE (e.g, IRES) of the first linear polynucleotide comprises no nucleotide or nucleoside modifications. In some embodiments, the noncoding or coding region of the first linear polynucleotide comprises no nucleotide or nucleoside modifications. In some embodiments, the 5′ and/or 3′ intron fragment of the first linear polynucleotide comprises no nucleotide or nucleoside modifications. In some embodiments, the 5′ and/or 3′ exon fragment of the first linear polynucleotide comprises no nucleotide or nucleoside modifications. In some embodiments, the spacer of the first linear polynucleotide comprises no nucleotide or nucleoside modifications. In some embodiments, the internal and/or external homology region of the first linear polynucleotide comprises no nucleotide or nucleoside modifications.

In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the second linear precursor are modified. In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the second linear precursor are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the second linear precursor are unmodified.

In some embodiments, in portions of the second linear polynucleotide, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the noncoding or coding region of the second linear polynucleotide are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the 5′ and/or 3′ intron fragment of the second linear polynucleotide are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the 5′ and/or 3′ exon fragment of the second linear polynucleotide are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the spacer of the second linear polynucleotide are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the internal and/or external homology region of the second linear polynucleotide are modified.

In some embodiments, in portions of the second linear polynucleotide, less than 10% of the nucleotides or nucleosides are modified. For example, in some embodiments, less than 10% of the nucleotides or nucleosides in the noncoding or coding region of the second linear polynucleotide are modified. In some embodiments, less than 10% of the nucleotides or nucleosides in the 5′ and/or 3′ intron fragment of the second linear polynucleotide are modified. In some embodiments, less than 10% of the nucleotides or nucleosides in the 5′ and/or 3′ exon fragment of the second linear polynucleotide is modified. In some embodiments, less than 10% of the nucleotides or nucleosides in the spacer of the second linear polynucleotide are modified. In some embodiments, less than 10% of the nucleotides or nucleosides in the internal and/or external homology region of the second linear polynucleotide are modified.

For example, in some embodiments, the noncoding or coding region of the second linear polynucleotide comprises no nucleotide or nucleoside modifications. In some embodiments, the 5′ intron fragment and/or 3′ intron fragment of the second linear polynucleotide comprises no nucleotide or nucleoside modifications. In some embodiments, the 5′ exon fragment and/or 3′ exon fragment of the second linear polynucleotide comprises no nucleotide or nucleoside modifications. In some embodiments, the spacer of the second linear polynucleotide comprises no nucleotide or nucleoside modifications. In some embodiments, the internal and/or external homology region of the second linear polynucleotide comprises no nucleotide or nucleoside modifications.

In some embodiments, in a first linear precursor of the present disclosure, the intervening region comprises no nucleotide or nucleoside modifications or is less than 10% modified; the TIE (e.g., IRES) comprises no nucleotide or nucleoside modifications or is less than 10% modified; the noncoding or coding region comprises no nucleotide or nucleoside modifications or is less than 10% modified; the 5′ and/or 3′ intron fragment comprises no nucleotide or nucleoside modifications or is less than 10% modified; the 5′ and/or 3′ exon fragment comprises no nucleotide or nucleoside modifications or is less than 10% modified; the spacer comprises no nucleotide or nucleoside modifications or is less than 10% modified; and/or the internal and/or external homology region comprises no nucleotide or nucleoside modifications or is less than 10% modified.

In some embodiments, in a second linear precursor of the present disclosure, the noncoding or coding region comprises no nucleotide or nucleoside modifications or is less than 10% modified; the 5′ intron fragment and/or 3′ intron fragment comprises no nucleotide or nucleoside modifications or is less than 10% modified; the 5′ exon fragment and/or 3′ exon fragment comprises no nucleotide or nucleoside modifications or is less than 10% modified; the spacer comprises no nucleotide or nucleoside modifications or is less than 10% modified; and/or the internal and/or external homology region comprises no nucleotide or nucleoside modifications or is less than 10% modified.

In some embodiments, incorporation of a nucleotide or nucleoside modification to a precursor RNA polynucleotide hinders or lowers the capacity of the circular RNA to circularize, splice, or express. In some embodiments, the precursor polynucleotide comprising no modified nucleotides and/or nucleosides maintains or improves circularization as compared to a precursor polynucleotide comprising one or more nucleotide or nucleoside modification. In some embodiments, the precursor polynucleotide comprising no modified nucleotides or nucleosides maintains circularization at greater than 50%, greater than 60%, greater than 70%, greater than 80%, greater than 90%, or greater than 100% (i.e., improves circularization), as compared to a corresponding precursor polynucleotide comprising one or more nucleotide or nucleoside modification.

In some embodiments, the polynucleotides comprising no nucleotide or nucleoside modifications, for an example a circular RNA, has comparable or reduced immunogenicity as compared to a polynucleotide comprising one or more nucleotide or nucleoside modification. In some embodiments, the circular RNAs described herein (i.e., a circular RNA polynucleotide comprising no nucleotide or nucleoside modification) exhibit immunogenicity that is reduced by about 10% to about 99%, for example reduced by about 10%, 15%, 20%, 25%, 30%, 35%, 40%, 45%, 50%, 55%, 60%, 65%, 70%, 75%, 80%, 85%, 90%, 95%, or 99% as compared to a corresponding circular RNA comprising one or more nucleotide or nucleoside modifications. In some embodiments, the polynucleotides comprising no modified nucleotides and/or modified nucleosides, for example a circular RNA, maintain or improve translation of a coding region as compared to a corresponding polynucleotide comprising one or more nucleotide or nucleoside modifications. In some embodiments, the polynucleotides comprising no modified nucleosides provide additional stability and resistance to immune activation. In some embodiments, for example, the polynucleotide comprising no modified A, C, G, or U nucleotide or nucleoside maintains expression at greater than 50%, greater than 60%, greater than 70%, greater than 80%, greater than 90%, or greater than 100%, as compared to a corresponding precursor polynucleotide comprising one or more nucleotide or nucleoside modifications. In some embodiments, the non-modified polynucleotides maintain expression at greater than 70% as compared to a corresponding polynucleotide comprising one or more nucleotide or nucleoside modifications. In some embodiments, the non-modified polynucleotides maintain expression at greater than 80% as compared to a corresponding polynucleotide comprising one or more nucleotide or nucleoside modifications. In some embodiments, the non-modified polynucleotides maintain expression at greater than 90% as compared to a corresponding polynucleotide comprising one or more nucleotide or nucleoside modifications. In some embodiments, the non-modified polynucleotides exhibit greater than 100% expression (i.e., improved expression) as compared to a corresponding polynucleotide comprising one or more nucleotide or nucleoside modifications. In some embodiments, the non-modified polynucleotides exhibit greater purification efficacy as compared to a corresponding polynucleotide comprising one or more nucleotide or nucleoside modifications. In some embodiments, for example, the polynucleotide comprising no modified A, C, G, or U nucleotide or nucleoside exhibits greater purification efficacy at greater than 50%, greater than 60%, greater than 70%, greater than 80%, greater than 90%, or greater than 100%, as compared to a corresponding precursor comprising one or more nucleotide or nucleoside modification.

### G. Codon Optimization

In particular embodiments, polynucleotides may be codon-optimized. A codon-optimized sequence may be one in which codons in a polynucleotide encoding a polypeptide have been substituted in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid. In some embodiments, a codon optimized polynucleotide may minimize ribozyme collisions and/or limit structural interference between the expression sequence and the core functional element. Codon optimization can be performed by methods known in the art.

### 3. Payloads—Coding Regions

In various embodiments, a provided polynucleotide (e.g., a DNA template, a linear precursor RNA polynucleotide, or a circular RNA polynucleotide) comprises one or more expression sequences or portions thereof. In some embodiments, the precursor RNA polynucleotide and circular RNA constructs comprise at least one expression sequence encoding a binding molecule. In certain embodiments, the precursor RNA polynucleotide and the circular RNA constructs comprise at least one expression sequence encoding a therapeutic protein and an IRES, wherein the IRES can facilitate expression of the protein when delivered in vivo. In some embodiments, the coding (or non-coding region) is a part of the intervening region or core functional element located in between the 5′ end and 3′ end of a linear precursor RNA polynucleotide and resultant circular RNA.

In some embodiments, the precursor RNA polynucleotide and circular RNA may encode for various therapeutic proteins, cytokines, immune checkpoint inhibitors, agonists, chimeric antigen receptors, inhibitory receptor agonists, one or more T-Cell Receptors, and/or B-cell Receptors that are available in the art. The chimeric proteins may also include, for example, recombinant fusion proteins, chimeric mutant protein, or other fusion proteins. In some embodiments, the circular RNA comprises more than 1 expression sequence, e.g., 2, 3, 4, or 5 expression sequences. In some embodiments, the circular RNA is a bicistronic RNA. In some embodiments, the bicistronic RNA is codon optimized. Exemplary bicistronic circular RNA are described in WO2021/189059A2, which is incorporated by reference herein in its entirety.

In some embodiments, the precursor RNA polynucleotide and circular RNA constructs comprise at least one expression sequence encoding an antigen, adjuvant, or adjuvant-like protein, e.g., from an infectious agent. In these embodiments, the circular RNA construct may be used as a vaccine.

In some embodiments, the expression sequence encodes a therapeutic protein. Nonlimiting examples of therapeutic proteins are listed in Table 2. In some embodiments, the scFv, heavy variable domain, light variable domain, heavy CDR sequences, and/or light CDR sequences of the therapeutic proteins listed in Table 2 may be used.

TABLE 2
Exemplary therapeutic proteins

| SEQ ID NO: | Payload | Sequence |
|---|---|---|
| 4019 | CD19 CAR | MLLLVTSLLLCELPHPAFLLIPDIQMTQTTSSLSASLGDRVTISCRASQDISK |
| | | YLNWYQQKPDGTVKLLIYHTSRLHSGVPSRFSGSGSGTDYSLTISNLEQEDI |
| | | ATYFCQQGNTLPYTFGGGTKLEITGSTSGSGKPGSGEGSTKGEVKLQESGP |
| | | GLVAPSQSLSVTCTVSGVSLPDYGVSWIRQPPRKGLEWLGVIWGSETTYY |
| | | NSALKSRLTIIKDNSKSQVFLKMNSLQTDDTAIYYCAKHYYYGGSYAMDY |
| | | WGQGTSVTVSSAAAIEVMYPPPYLDNEKSNGTIIHVKGKHLCPSPLFPGPS |
| | | KPFWVLVVVGGVLACYSLLVTVAFIIFWVKRGRKKLLYIFKQPFMRPVQT |
| | | TQEEDGCSCRFPEEEEGGCEL |
| 4020 | CD19 CAR | MLLLVTSLLLCELPHPAFLLIPDIQMTQTTSSLSASLGDRVTISCRASQDISK |
| | | YLNWYQQKPDGTVKLLIYHTSRLHSGVPSRFSGSGSGTDYSLTISNLEQEDI |
| | | ATYFCQQGNTLPYTFGGGTKLEITGSTSGSGKPGSGEGSTKGEVKLQESGP |
| | | GLVAPSQSLSVTCTVSGVSLPDYGVSWIRQPPRKGLEWLGVIWGSETTYY |
| | | NSALKSRLTIIKDNSKSQVFLKMNSLQTDDTAIYYCAKHYYYGGSYAMDY |
| | | WGQGTSVTVSSAAAIEVMYPPPYLDNEKSNGTIIHVKGKHLCPSPLFPGPS |
| | | KPFWVLVVVGGVLACYSLLVTVAFIIFWVRSKRSRLLHSDYMNMTPRRPG |
| | | PTRKHYQPYAPPRDFAAYRS |
| 4021 | CD19 CAR | MLLLVTSLLLCELPHPAFLLIPDIQMTQTTSSLSASLGDRVTISCRASQDISK |
| | | YLNWYQQKPDGTVKLLIYHTSRLHSGVPSRFSGSGSGTDYSLTISNLEQEDI |
| | | ATYFCQQGNTLPYTFGGGTKLEITGSTSGSGKPGSGEGSTKGEVKLQESGP |
| | | GLVAPSQSLSVTCTVSGVSLPDYGVSWIRQPPRKGLEWLGVIWGSETTYY |
| | | NSALKSRLTIIKDNSKSQVFLKMNSLQTDDTAIYYCAKHYYYGGSYAMDY |
| | | WGQGTSVTVSSAAAIEVMYPPPYLDNEKSNGTIIHVKGKHLCPSPLFPGPS |
| | | KPFWVLVVVGGVLACYSLLVTVAFIIFWVRSKRSRLLHSDYMNMTPRRPG |
| | | PTRKHYQPYAPPRDFAAYRSRVKFSRSADAPAYQQGQNQLYNELNLGRR |
| | | EEYDVLDKRRGRDPEMGGKPRRKNPQEGLYNELQKDKMAEAYSEIGMK |
| | | GERRRGKGHDGLYQGLSTATKDTYDALHMQALPPR |
| 4022 | CD19 CAR | MLLLVTSLLLCELPHPAFLLIPDIQMTQTTSSLSASLGDRVTISCRASQDISK |
| | | YLNWYQQKPDGTVKLLIYHTSRLHSGVPSRFSGSGSGTDYSLTISNLEQEDI |
| | | ATYFCQQGNTLPYTFGGGTKLEITGSTSGSGKPGSGEGSTKGEVKLQESGP |
| | | GLVAPSQSLSVTCTVSGVSLPDYGVSWIRQPPRKGLEWLGVIWGSETTYY |
| | | NSALKSRLTIIKDNSKSQVFLKMNSLQTDDTAIYYCAKHYYYGGSYAMDY |
| | | WGQGTSVTVSSAAAIEVMYPPPYLDNEKSNGTIIHVKGKHLCPSPLFPGPS |
| | | KPFWVLVVVGGVLACYSLLVTVAFIIFWVRVKFSRSADAPAYQQGQNQL |
| | | YNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 4023 | CD19 CAR | MALPVTALLLPLALLLHAARPDIQMTQTTSSLSASLGDRVTISCRASQDISK |
| | | YLNWYQQKPDGTVKLLIYHTSRLHSGVPSRFSGSGSGTDYSLTISNLEQEDI |
| | | ATYFCQQGNTLPYTFGGGTKLEITGGGGSGGGGSGGGGSEVKLQESGPGL |
| | | VAPSQSLSVTCTVSGVSLPDYGVSWIRQPPRKGLEWLGVIWGSETTYYNS |
| | | ALKSRLTIIKDNSKSQVFLKMNSLQTDDTAIYYCAKHYYYGGSYAMDYW |
| | | GQGTSVTVSSTTTPAPRPPTPAPTIASQPLSLRPEACRPAAGGAVHTRGLDF |
| | | ACDIYIWAPLAGTCGVLLLSLVITLYCKRGRKKLLYIFKQPFMRPVQTTQE |
| | | EDGCSCRFPEEEEGGCELRVKFSRSADAPAYQQGQNQLYNELNLGRREEY |
| | | DVLDKRRGRDPEMGGKPRRKNPQEGLYNELQKDKMAEAYSEIGMKGER |
| | | RRGKGHDGLYQGLSTATKDTYDALHMQALPPR |
| 4024 | CD19 CAR | MALPVTALLLPLALLLHAARPDIQMTQTTSSLSASLGDRVTISCRASQDISK |
| | | YLNWYQQKPDGTVKLLIYHTSRLHSGVPSRFSGSGSGTDYSLTISNLEQEDI |
| | | ATYFCQQGNTLPYTFGGGTKLEITGGGGSGGGGSGGGGSEVKLQESGPGL |
| | | VAPSQSLSVTCTVSGVSLPDYGVSWIRQPPRKGLEWLGVIWGSETTYYNS |
| | | ALKSRLTIIKDNSKSQVFLKMNSLQTDDTAIYYCAKHYYYGGSYAMDYW |
| | | GQGTSVTVSSTTTPAPRPPTPAPTIASQPLSLRPEACRPAAGGAVHTRGLDF |
| | | ACDIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRREEY |
| | | DVLDKRRGRDPEMGGKPRRKNPQEGLYNELQKDKMAEAYSEIGMKGER |
| | | RRGKGHDGLYQGLSTATKDTYDALHMQALPPR |
| 4025 | CD22 | MLLLVTSLLLCELPHPAFLLIPQVQLQQSGPGLVKPSQTLSLTCAISGDSVSS |
| | | NSAAWNWIRQSPSRGLEWLGRTYYRSKWYNDYAVSVKSRITINPDTSKN |
| | | QFSLQLNSVTPEDTAVYYCAREVTGDLEDAFDIWGQGTMVTVSSGGGGS |
| | | DIQMTQSPSSLSASVGDRVTITCRASQTIWSYLNWYQQRPGKAPNLLIYAA |
| | | SSLQSGVPSRFSGRGSGTDFTLTISSLQAEDFATYYCQQSYSIPQTFGQGTK |
| | | LEIKSGTTTPAPRPPTPAPTIASQPLSLRPEACRPAAGGAVHTRGLDFACDIY |
| | | IWAPLAGTCGVLLLSLVITLYCKRGRKKLLYIFKQPFMRPVQTTQEEDGCS |
| | | CRFPEEEEGGCELRVKFSRSADAPAYKQGQNQLYNELNLGRREEYDVLDK |
| | | RRGRDPEMGGKPRRKNPQEGLYNELQKDKMAEAYSEIGMKGERRRGKG |
| | | HDGLYQGLSTATKDTYDALHMQALPPR |
| 4026 | CD22 | QVQLQQSGPGMVKPSQTLSLTCAISGDSVSSNSVAWNWIRQSPSRGLEWL |
| | | GRTYYRSTWYNDYAVSMKSRITINPDTNKNQFSLQLNSVTPEDTAVYYCA |
| | | REVTGDLEDAFDIWGQGTMVTVSSGGGGSGGGGSGGGGSDIQMIQSPSSL |
| | | SASVGDRVTITCRASQTIWSYLNWYRQRPGEAPNLLIYAASSLQSGVPSRF |
| | | SGRGSGTDFTLTISSLQAEDFATYYCQQSYSIPQTFGQGTKLEIK |
| 4027 | CD20 | METDTLLLWVLLLWVPGSTGEIVLTQSPATLSLSPGERATMTCRASSSVNY |
| | | MDWYQQKPGQAPRPWIYATSNLASGVPARFSGSGSGTDFTLTISSLEPEDF |
| | | AVYYCQQWSFNPPTFGGGTKVEIKGSTSGGGSGGGSGGGGSSQVQLVQSG |
| | | AEVKKPGASVKVSCKASGYTFTSYNMHWVRQAPGQGLEWIGAIYPGNGD |
| | | TSYNQKFKGKATLTRDTSTSTVYMELSSLRSEDTAVYYCARSNYYGSSYW |
| | | FFDVWGQGTTVTVSSTTTPAPRPPTPAPTIASQPLSLRPEACRPAAGGAVHT |
| | | RGLDFACDFWLPIGCAAFVVVCILGCILICWLTKKKYSSSVHDPNGEYMF |
| | | MRAVNTAKKSRLTDVTLRVKFSRSADAPAYKQGQNQLYNELNLGRREEY |
| | | DVLDKRRGRDPEMGGKPRRKNPQEGLYNELQKDKMAEAYSEIGMKGER |
| | | RRGKGHDGLYQGLSTATKDTYDALHMQALP |
| 4028 | BCMA CAR | MALPVTALLLPLALLLHAARPDIVLTQSPASLAVSLGERATINCRASESVSV |
| | | IGAHLIHWYQQKPGQPPKLLIYLASNLETGVPARFSGSGSGTDFTLTISSLQ |
| | | AEDAAIYYCLQSRIFPRTFGQGTKLEIKGSTSGSGKPGSGEGSTKGQVQLV |
| | | QSGSELKKPGASVKVSCKASGYTFTDYSINWVRQAPGQGLEWMGWINTE |
| | | TREPAYAYDFRGRFVFSLDTSVSTAYLQISSLKAEDTAVYYCARDYSYAM |
| | | DYWGQGTLVTVSSAAATTTPAPRPPTPAPTIASQPLSLRPEACRPAAGGAV |
| | | HTRGLDFACDIYIWAPLAGTCGVLLLSLVITLYCKRGRKKLLYIFKQPFMR |
| | | PVQTTQEEDGCSCRFPEEEEGGCELRVKFSRSADAPAYQQGQNQLYNELN |
| | | LGRREEYDVLDKRRGRDPEMGGKPRRKNPQEGLYNELQKDKMAEAYSEI |
| | | GMKGERRRGKGHDGLYQGLSTATKDTYDALHMQALPPR |
| | | In some embodiments, the BCMA CAR may be chosen from any of the anti-BCMA |
| | | CARs disclosed in U.S. patent application U.S. 2021/0128618A1. |
| 4029-4030 | MAGE-A4 | TCR alpha chain: |
| | TCR | KNQVEQSPQSLIILEGKNCTLQCNYTVSPFSNLRWYKQDTGRGPVSLTIMT |
| | | FSENTKSNGRYTATLDADTKQSSLHITASQLSDSASYICVVNHSGGSYIPTF |
| | | GRGTSLIVHPYIQKPDPAVYQLRDSKSSDKSVCLFTDFDSQTNVSQSKDSD |
| | | VYITDKTVLDMRSMDFKSNSAVAWSNKSDFACANAFNNSIIPEDTFFPSPE |
| | | SS (SEQ ID NO: 4029) |
| | | TCR beta chain: |
| | | DVKVTQSSRYLVKRTGEKVFLECVQDMDHENMFWYRQDPGLGLRLIYFS |
| | | YDVKMKEKGDIPEGYSVSREKKERFSLILESASTNQTSMYLCASSFLMTSG |
| | | DPYEQYFGPGTRLTVTEDLKNVFPPEVAVFEPSEAEISHTQKATLVCLATG |
| | | FYPDHVELSWWVNGKEVHSGVSTDPQPLKEQP |
| | | ALNDSRYCLSSRLRVSATFWQNPRNHFRCQVQFYGLSENDEWTQDRAKP |
| | | VTQIVSAEAWGRAD (SEQ ID NO: 4030) |
| 4031 | NY-ESO | TCRalpha extracellular sequence |
| and | TCR | MQEVTQIPAALSVPEGENLVLNCSFTDSAIYNLQWFRQDPGKGLTSLLLIQ |
| 4032 | | SSQREQTSGRLNASLDKSSGRSTLYIAASQPGDSATYLCAVRPTSGGSYIPT |
| | | FGRGTSLIVHPY (SEQ ID NO: 4031) |
| | | TCRbeta extracellular sequence |
| | | MGVTQTPKFQVLKTGQSMTLQCAQDMNHEYMSWYRQDPGMGLRLIHYS |
| | | VGAGITDQGEVPNGYNVSRSTTEDFPLRLLSAAPSQTSVYFCASSYVGNTG |
| | | ELFFGEGSRLTVL (SEQ ID NO: 4032) |
| 4033 | EPO | APPRLICDSRVLERYLLEAKEAENITTGCAEHCSLNENITVPDTKVNFYAW |
| | | KRMEVGQQAVEVWQGLALLSEAVLRGQALLVNSSQPWEPLQLHVDKAV |
| | | SGLRSLTTLLRALGAQKEAISPPDAASAAPLRTITADTFRKLFRVYSNFLRG |
| | | KLKLYTGEACRTGDR |
| 4034 | PAH | MSTAVLENPGLGRKLSDFGQETSYIEDNCNQNGAISLIFSLKEEVGALAKV |
| | | LRLFEENDVNLTHIESRPSRLKKDEYEFFTHLDKRSLPALTNIIKILRHDIGA |
| | | TVHELSRDKKKDTVPWFPRTIQELDRFANQILSYGAELDADHPGFKDPVY |
| | | RARRKQFADIAYNYRHGQPIPRVEYMEEEKKTWGTVFKTLKSLYKTHAC |
| | | YEYNHIFPLLEKYCGFHEDNIPQLEDVSQFLQTCTGFRLRPVAGLLSSRDFL |
| | | GGLAFRVFHCTQYIRHGSKPMYTPEPDICHELLGHVPLFSDRSFAQFSQEIG |
| | | LASLGAPDEYIEKLATIYWFTVEFGLCKQGDSIKAYGAGLLSSFGELQYCL |
| | | SEKPKLLPLELEKTAIQNYTVTEFQPLYYVAESFNDAKEKVRNFAATIPRPF |
| | | SVRYDPYTQRIEVLDNTQQLKILADSINSEIGILCSALQKIK |
| 4035 | CPS1 | LSVKAQTAHIVLEDGTKMKGYSFGHPSSVAGEVVFNTGLGGYPEAITDPA |
| | | YKGQILTMANPIIGNGGAPDTTALDELGLSKYLESNGIKVSGLLVLDYSKD |
| | | YNHWLATKSLGQWLQEEKVPAIYGVDTRMLTKIIRDKGTMLGKIEFEGQP |
| | | VDFVDPNKQNLIAEVSTKDVKVYGKGNPTKVVAVDCGIKNNVIRLLVKR |
| | | GAEVHLVPWNHDFTKMEYDGILIAGGPGNPALAEPLIQNVRKILESDRKEP |
| | | LFGISTGNLITGLAAGAKTYKMSMANRGQNQPVLNITNKQAFITAQNHGY |
| | | ALDNTLPAGWKPLFVNVNDQTNEGIMHESKPFFAVQFHPEVTPGPIDTEYL |
| | | FDSFFSLIKKGKATTITSVLPKPALVASRVEVSKVLILGSGGLSIGQAGEFDY |
| | | SGSQAVKAMKEENVKTVLMNPNIASVQTNEVGLKQADTVYFLPITPQFVT |
| | | EVIKAEQPDGLILGMGGQTALNCGVELFKRGVLKEYGVKVLGTSVESIMA |
| | | TEDRQLFSDKLNEINEKIAPSFAVESIEDALKAADTIGYPVMIRSAYALGGL |
| | | GSGICPNRETLMDLSTKAFAMTNQILVEKSVTGWKEIEYEVVRDADDNCV |
| | | TVCNMENVDAMGVHTGDSVVVAPAQTLSNAEFQMLRRTSINVVRHLGIV |
| | | GECNIQFALHPTSMEYCIIEVNARLSRSSALASKATGYPLAFIAAKIALGIPL |
| | | PEIKNVVSGKTSACFEPSLDYMVTKIPRWDLDRFHGTSSRIGSSMKSVGEV |
| | | MAIGRTFEESFQKALRMCHPSIEGFTPRLPMNKEWPSNLDLRKELSEPSSTR |
| | | IYAIAKAIDDNMSLDEIEKLTYIDKWFLYKMRDILNMEKTLKGLNSESMTE |
| | | ETLKRAKEIGFSDKQISKCLGLTEAQTRELRLKKNIHPWVKQIDTLAAEYPS |
| | | VINYLYVTYNGQEHDVNFDDHGMMVLGCGPYHIGSSVEFDWCAVSSIRT |
| | | LRQLGKKTVVVNCNPETVSTDFDECDKLYFEELSLERILDIYHQEACGGCII |
| | | SVGGQIPNNLAVPLYKNGVKIMGTSPLQIDRAEDRSIFSAVLDELKVAQAP |
| | | WKAVNTLNEALEFAKSVDYPCLLRPSYVLSGSAMNVVFSEDEMKKFLEE |
| | | ATRVSQEHPVVLTKFVEGAREVEMDAVGKDGRVISHAISEHVEDAGVHSG |
| | | DATLMLPTQTISQGAIEKVKDATRKIAKAFAISGPFNVQFLVKGNDVLVIE |
| | | CNLRASRSFPFVSKTLGVDFIDVATKVMIGENVDEKHLPTLDHPIIPADYVA |
| | | IKAPMFSWPRLRDADPILRCEMASTGEVACFGEGIHTAFLKAMLSTGFKIP |
| | | QKGILIGIQQSFRPRFLGVAEQLHNEGFKLFATEATSDWLNANNVPATPVA |
| | | WPSQEGQNPSLSSIRKLIRDGSIDLVINLPNNNTKFVHDNYVIRRTAVDSGIP |
| | | LLTNFQVTKLFAEAVQKSRKVDSKSLFHYRQYSAGKAA |
| 4036 | Cas9 | MKRNYILGLDIGITSVGYGIIDYETRDVIDAGVRLFKEANVENNEGRRSKR |
| | | GARRLKRRRRHRIQRVKKLLFDYNLLTDHSELSGINPYEARVKGLSQKLSE |
| | | EEFSAALLHLAKRRGVHNVNEVEEDTGNELSTKEQISRNSKALEEKYVAE |
| | | LQLERLKKDGEVRGSINRFKTSDYVKEAKQLLKVQKAYHQLDQSFIDTYI |
| | | DLLETRRTYYEGPGEGSPFGWKDIKEWYEMLMGHCTYFPEELRSVKYAY |
| | | NADLYNALNDLNNLVITRDENEKLEYYEKFQIIENVFKQKKKPTLKQIAKE |
| | | ILVNEEDIKGYRVTSTGKPEFTNLKVYHDIKDITARKEIIENAELLDQIAKIL |
| | | TIYQSSEDIQEELTNLNSELTQEEIEQISNLKGYTGTHNLSLKAINLILDELW |
| | | HTNDNQIAIFNRLKLVPKKVDLSQQKEIPTTLVDDFILSPVVKRSFIQSIKVI |
| | | NAIIKKYGLPNDIIIELAREKNSKDAQKMINEMQKRNRQTNERIEEIIRTTGK |
| | | ENAKYLIEKIKLHDMQEGKCLYSLEAIPLEDLLNNPFNYEVDHIIPRSVSFD |
| | | NSFNNKVLVKQEENSKKGNRTPFQYLSSSDSKISYETFKKHILNLAKGKGR |
| | | ISKTKKEYLLEERDINRFSVQKDFINRNLVDTRYATRGLMNLLRSYFRVNN |
| | | LDVKVKSINGGFTSFLRRKWKFKKERNKGYKHHAEDALIIANADFIFKEW |
| | | KKLDKAKKVMENQMFEEKQAESMPEIETEQEYKEIFITPHQIKHIKDFKDY |
| | | KYSHRVDKKPNRELINDTLYSTRKDDKGNTLIVNNLNGLYDKDNDKLKK |
| | | LINKSPEKLLMYHHDPQTYQKLKLIMEQYGDEKNPLYKYYEETGNYLTKY |
| | | SKKDNGPVIKKIKYYGNKLNAHLDITDDYPNSRNKVVKLSLKPYRFDVYL |
| | | DNGVYKFVTVKNLDVIKKENYYEVNSKCYEEAKKLKKISNQAEFIASFYN |
| | | NDLIKINGELYRVIGVNNDLLNRIEVNMIDITYREYLENMNDKRPPRIIKTIA |
| | | SKTQSIKKYSTDILGNLYEVKSKKHPQIIKKG |
| 4037 | ADAMTS13 | AAGGILHLELLVAVGPDVFQAHQEDTERYVLTNLNIGAELLRDPSLGAQF |
| | | RVHLVKMVILTEPEGAPNITANLTSSLLSVCGWSQTINPEDDTDPGHADLV |
| | | LYITRFDLELPDGNRQVRGVTQLGGACSPTWSCLITEDTGFDLGVTIAHEIG |
| | | HSFGLEHDGAPGSGCGPSGHVMASDGAAPRAGLAWSPCSRRQLLSLLSAG |
| | | RARCVWDPPRPQPGSAGHPPDAQPGLYYSANEQCRVAFGPKAVACTFAR |
| | | EHLDMCQALSCHTDPLDQSSCSRLLVPLLDGTECGVEKWCSKGRCRSLVE |
| | | LTPIAAVHGRWSSWGPRSPCSRSCGGGVVTRRRQCNNPRPAFGGRACVGA |
| | | DLQAEMCNTQACEKTQLEFMSQQCARTDGQPLRSSPGGASFYHWGAAVP |
| | | HSQGDALCRHMCRAIGESFIMKRGDSFLDGTRCMPSGPREDGTLSLCVSGS |
| | | CRTFGCDGRMDSQQVWDRCQVCGGDNSTCSPRKGSFTAGRAREYVTFLT |
| | | VTPNLTSVYIANHRPLFTHLAVRIGGRYVVAGKMSISPNTTYPSLLEDGRV |
| | | EYRVALTEDRLPRLEEIRIWGPLQEDADIQVYRRYGEEYGNLTRPDITFTYF |
| | | QPKPRQAWVWAAVRGPCSVSCGAGLRWVNYSCLDQARKELVETVQCQG |
| | | SQQPPAWPEACVLEPCPPYWAVGDFGPCSASCGGGLRERPVRCVEAQGSL |
| | | LKTLPPARCRAGAQQPAVALETCNPQPCPARWEVSEPSSCTSAGGAGLAL |
| | | ENETCVPGADGLEAPVTEGPGSVDEKLPAPEPCVGMSCPPGWGHLDATSA |
| | | GEKAPSPWGSIRTGAQAAHVWTPAAGSCSVSCGRGLMELRFLCMDSALR |
| | | VPVQEELCGLASKPGSRREVCQAVPCPARWQYKLAACSVSCGRGVVRRIL |
| | | YCARAHGEDDGEEILLDTQCQGLPRPEPQEACSLEPCPPRWKVMSLGPCS |
| | | ASCGLGTARRSVACVQLDQGQDVEVDEAACAALVRPEASVPCLIADCTY |
| | | RWHVGTWMECSVSCGDGIQRRRDTCLGPQAQAPVPADFCQHLPKPVTVR |
| | | GCWAGPCVGQGTPSLVPHEEAAAPGRTTATPAGASLEWSQARGLLFSPAP |
| | | QPRRLLPGPQENSVQSSACGRQHLEPTGTIDMRGPGQADCAVAIGRPLGEV |
| | | VTLRVLESSLNCSAGDMLLLWGRLTWRKMCRKLLDMTFSSKTNTLVVRQ |
| | | RCGRPGGGVLLRYGSQLAPETFYRECDMQLFGPWGEIVSPSLSPATSNAG |
| | | GCRLFINVAPHARIAIHALATNMGAGTEGANASYILIRDTHSLRTTAFHGQ |
| | | QVLYWESESSQAEMEFSEGFLKAQASLRGQYWTLQSWVPEMQDPQSWK |
| | | GKEGT |
| 4038 | FOXP3 | MPNPRPGKPSAPSLALGPSPGASPSWRAAPKASDLLGARGPGGTFQGRDL |
| | | RGGAHASSSSLNPMPPSQLQLPTLPLVMVAPSGARLGPLPHLQALLQDRPH |
| | | FMHQLSTVDAHARTPVLQVHPLESPAMISLTPPTTATGVFSLKARPGLPPGI |
| | | NVASLEWVSREPALLCTFPNPSAPRKDSTLSAVPQSSYPLLANGVCKWPG |
| | | CEKVFEEPEDFLKHCQADHLLDEKGRAQCLLQREMVQSLEQQLVLEKEKL |
| | | SAMQAHLAGKMALTKASSVASSDKGSCCIVAAGSQGPVVPAWSGPREAP |
| | | DSLFAVRRHLWGSHGNSTFPEFLHNMDYFKFHNMRPPFTYATLIRWAILE |
| | | APEKQRTLNEIYHWFTRMFAFFRNHPATWKNAIRHNLSLHKCFVRVESEK |
| | | GAVWTVDELEFRKKRSQRPSRCSNPTPGP |
| 4039 | IL-10 | SPGQGTQSENSCTHFPGNLPNMLRDLRDAFSRVKTFFQMKDQLDNLLLKE |
| | | SLLEDFKGYLGCQALSEMIQFYLEEVMPQAENQDPDIKAHVNSLGENLKT |
| | | LRLRLRRCHRFLPCENKSKAVEQVKNAFNKLQEKGIYKAMSEFDIFINYIE |
| | | AYMTMKIRN |
| 4040 | IL-2 | APTSSSTKKTQLQLEHLLLDLQMILNGINNYKNPKLTRMLTFKFYMPKKA |
| | | TELKHLQCLEEELKPLEEVLNLAQSKNFHLRPRDLISNINVIVLELKGSETTF |
| | | MCEYADETATIVEFLNRWITFCQSIISTLT |

In some embodiments, the therapeutic protein is selected from a CD19-targted chimeric antigen receptor (CAR), a BCMA-targeted CAR, MAGE-A4 T-cell receptor (TCR), NY-ESO TCR, erythropoietin (EPO), phenylalanine hydroxylase (PAH), carbamoyl phosphate synthetase I (CPS1), Cas9, ADAMTS13, FOXP3, IL-10, or IL-2. Exemplary sequences are provided herein in Table 2. In some embodiments, the target amino acid has an amino acid sequence that is identical to or at least 80%, 85%, 90%, 91%, 92%, 93%, 94%, 95%, 96%, 97%, 98%, or 99% identical to a sequence selected from SEQ ID NOS: 4019-4040, or a fragment thereof.

In some embodiments, the expression sequence encodes a therapeutic protein. In some embodiments, the expression sequence encodes a cytokine, e.g., IL-12p70, IL-15, IL-2, IL-18, IL-21, IFN-α, IFN-β, IL-10, TGF-beta, IL-4, or IL-35, or a functional fragment thereof. In some embodiments, the expression sequence encodes an immune checkpoint inhibitor. In some embodiments, the expression sequence encodes an agonist (e.g., a TNFR family member such as CD137L, OX40L, ICOSL, LIGHT, or CD70). In some embodiments, the expression sequence encodes a chimeric antigen receptor. In some embodiments, the expression sequence encodes an inhibitory receptor agonist (e.g., PDL1, PDL2, Galectin-9, VISTA, B7H4, or MHCII) or inhibitory receptor (e.g., PD1, CTLA4, TIGIT, LAG3, or TIM3). In some embodiments, the expression sequence encodes an inhibitory receptor antagonist. In some embodiments, the expression sequence encodes one or more TCR chains (alpha and beta chains or gamma and delta chains). In some embodiments, the expression sequence encodes a secreted T cell or immune cell engager (e.g., a bispecific antibody such as BiTE, targeting, e.g., CD3, CD137, or CD28 and a tumor-expressed protein e.g., CD19, CD20, or BCMA etc.). In some embodiments, the expression sequence encodes a transcription factor (e.g., FOXP3, HELIOS, TOX1, or TOX2). In some embodiments, the expression sequence encodes an immunosuppressive enzyme (e.g., IDO or CD39/CD73). In some embodiments, the expression sequence encodes a GvHD (e.g., anti-HLA-A2 CAR-Tregs).

In some embodiments, a provided polynucleotide encodes a protein that is made up of subunits that are encoded by more than one gene. For example, the protein may be a heterodimer, wherein each chain or subunit of the protein is encoded by a separate gene. It is possible that more than one provided polynucleotides (e.g., circular RNA polynucleotides) are delivered in the transfer vehicle and each polynucleotide encodes a separate subunit of the protein. In certain embodiments, polynucleotides encoding the individual subunits may be administered in separate transfer vehicles. Alternatively, a single polynucleotide (e.g., circular RNA polynucleotide) may be engineered to encode more than one subunit.

### A. Antigen-Recognition Receptors

a. Chimeric Antigen Receptors (Cars)

In some embodiments, a provided polynucleotide (e.g., a DNA template, a linear precursor RNA polynucleotide, or a circular RNA polynucleotide) encodes one or more chimeric antigen receptors (CARs). CARs are genetically-engineered receptors. These engineered receptors may be inserted into and expressed by immune cells, including T cells via circular RNA as described herein. With a CAR, a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell. In some embodiments, the CAR encoded by the polynucleotide comprises (i) an antigen-binding molecule that specifically binds to a target antigen, (ii) a hinge domain, a transmembrane domain, and an intracellular domain, and (iii) an activating domain.

In some embodiments, an orientation of the CARs in accordance with the disclosure comprises an antigen binding domain (such as an scFv) in tandem with a costimulatory domain and an activating domain. The costimulatory domain may comprise one or more of an extracellular portion, a transmembrane portion, and an intracellular portion. In other embodiments, multiple costimulatory domains may be utilized in tandem.

i. Antigen Binding Domain

CARs may be engineered to bind to an antigen (such as a cell-surface antigen) by incorporating an antigen binding molecule that interacts with that targeted antigen. In some embodiments, the antigen binding molecule is an antibody fragment thereof, e.g., one or more single chain antibody fragment (scFv). An scFv is a single chain antibody fragment having the variable regions of the heavy and light chains of an antibody linked together. See U.S. Pat. Nos. 7,741,465, and 6,319,494, as well as Eshhar et al., Cancer Immunol Immunotherapy (1997) 45: 131-136. An scFv retains the parent antibody's ability to specifically interact with target antigen. scFvs are useful in chimeric antigen receptors because they may be engineered to be expressed as part of a single chain along with the other CAR components. Id. See also Krause et al., J. Exp. Med., Volume 188, No. 4, 1998 (619-626); Finney et al., Journal of Immunology, 1998, 161: 2791-2797. It will be appreciated that the antigen binding molecule is typically contained within the extracellular portion of the CAR such that it is capable of recognizing and binding to the antigen of interest. Bispecific and multispecific CARs are contemplated within the scope of the disclosure, with specificity to more than one target of interest.

In some embodiments, the antigen binding molecule comprises a single chain, wherein the heavy chain variable region and the light chain variable region are connected by a linker. In some embodiments, the VH is located at the N terminus of the linker and the VL is located at the C terminus of the linker. In other embodiments, the VL is located at the N terminus of the linker and the VH is located at the C terminus of the linker. In some embodiments, the linker comprises at least about 5, at least about 8, at least about 10, at least about 13, at least about 15, at least about 18, at least about 20, at least about 25, at least about 30, at least about 35, at least about 40, at least about 45, at least about 50, at least about 60, at least about 70, at least about 80, at least about 90, or at least about 100 amino acids.

In some embodiments, the antigen binding molecule comprises a nanobody. In some embodiments, the antigen binding molecule comprises a DARPin. In some embodiments, the antigen binding molecule comprises an anticalin or other synthetic protein capable of specific binding to target protein.

In some embodiments, the CAR comprises an antigen binding domain specific for an antigen selected from the group CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GaINAca-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin, Interleukin 11 receptor alpha (IL-I IRa), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, HER2, HER3, Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WT1), Cancer/testis antigen 1 (NY-ESO-1), Cancer/testis antigen 2 (LAGE-la), MAGE family members (including MAGE-A1, MAGE-A3 and MAGE-A4), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, surviving, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), MUC16, 5T4, 8H9, ανβθ integrin, ανβ6 integrin, alphafetoprotein (AFP), B7-H6, ca-125, CA9, CD44, CD44v7/8, CD52, E-cadherin, EMA (epithelial membrane antigen), epithelial glycoprotein-2 (EGP-2), epithelial glycoprotein-40 (EGP-40), ErbB4, epithelial tumor antigen (ETA), folate binding protein (FBP), kinase insert domain receptor (KDR), k-light chain, L1 cell adhesion molecule, MUC18, NKG2D, oncofetal antigen (h5T4), tumor/testis-antigen 1B, GAGE, GAGE-1, BAGE, SCP-1, CTZ9, SAGE, CAGE, CT10, MART-1, immunoglobulin lambda-like polypeptide 1 (IGLL1), Hepatitis B Surface Antigen Binding Protein (HBsAg), viral capsid antigen (VCA), early antigen (EA), EBV nuclear antigen (EBNA), HHV-6 p41 early antigen, HHV-6B U94 latent antigen, HHV-6B p98 late antigen, cytomegalovirus (CMV) antigen, large T antigen, small T antigen, adenovirus antigen, respiratory syncytial virus (RSV) antigen, haemagglutinin (HA), neuraminidase (NA), parainfluenza type 1 antigen, parainfluenza type 2 antigen, parainfluenza type 3 antigen, parainfluenza type 4 antigen, Human Metapneumovirus (HMPV) antigen, hepatitis C virus (HCV) core antigen, HIV p24 antigen, human T-cell lympotrophic virus (HTLV-1) antigen, Merkel cell polyoma virus small T antigen, Merkel cell polyoma virus large T antigen, Kaposi sarcoma-associated herpesvirus (KSHV) lytic nuclear antigen and KSHV latent nuclear antigen.

In some embodiments, the circular RNA constructs and related pharmaceutical compositions comprise the expression sequences described in Table 2, above. In some embodiments, the circular RNA constructs and related pharmaceutical compositions disclosed herein comprise an expression sequence that is at least 80%, 85%, 90%, 91%, 92%, 93%, 94%, 95%, 96%, 97%, 98%, or 99% identical to a sequence in Table 2, wherein the codon sequence produces a protein having the desired sequence.

ii. Hinge Spacer Domain

In some embodiments, a CAR of the instant disclosure comprises a hinge or spacer domain. In some embodiments, the hinge/spacer domain may comprise a truncated hinge/spacer domain (THD) the THD domain is a truncated version of a complete hinge/spacer domain (“CHD”). In some embodiments, an extracellular domain is from or derived from (e.g., comprises all or a fragment of) ErbB2, glycophorin A (GpA), CD2, CD3 delta, CD3 epsilon, CD3 gamma, CD4, CD7, CD8a, CD8[T CDl la (IT GAL), CDl lb (IT GAM), CDl lc (ITGAX), CDl ld (IT GAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAMS), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (0X40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRT AM), CD357 (TNFRSF18), inducible T cell co-stimulator (ICOS), LFA-1 (CDl la/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and fragments or combinations thereof. A hinge or spacer domain may be derived either from a natural or from a synthetic source.

In some embodiments, a hinge or spacer domain is positioned between an antigen binding molecule (e.g., an scFv) and a transmembrane domain. In this orientation, the hinge/spacer domain provides distance between the antigen binding molecule and the surface of a cell membrane on which the CAR is expressed. In some embodiments, a hinge or spacer domain is from or derived from an immunoglobulin. In some embodiments, a hinge or spacer domain is selected from the hinge/spacer regions of IgGl, IgG2, IgG3, IgG4, IgA, IgD, IgE, IgM, or a fragment thereof. In some embodiments, a hinge or spacer domain comprises, is from, or is derived from the hinge/spacer region of CD8 alpha. In some embodiments, a hinge or spacer domain comprises, is from, or is derived from the hinge/spacer region of CD28. In some embodiments, a hinge or spacer domain comprises a fragment of the hinge/spacer region of CD8 alpha or a fragment of the hinge/spacer region of CD28, wherein the fragment is anything less than the whole hinge/spacer region. In some embodiments, the fragment of the CD8 alpha hinge/spacer region or the fragment of the CD28 hinge/spacer region comprises an amino acid sequence that excludes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids at the N-terminus or C-Terminus, or both, of the CD8 alpha hinge/spacer region, or of the CD28 hinge/spacer region.

in. Transmembrane Domain

The CAR of the present disclosure may further comprise a transmembrane domain and/or an intracellular signaling domain. The transmembrane domain may be designed to be fused to the extracellular domain of the CAR. It may similarly be fused to the intracellular domain of the CAR. In some embodiments, the transmembrane domain that naturally is associated with one of the domains in a CAR is used. In some instances, the transmembrane domain may be selected or modified (e.g., by an amino acid substitution) to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein.

Transmembrane regions may be derived from (i.e. comprise) a receptor tyrosine kinase (e.g., ErbB2), glycophorin A (GpA), 4-1BB/CD137, activating NK cell receptors, an immunoglobulin protein, B7-H3, BAFFR, BFAME (SEAMF8), BTEA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD1 la, CD1 lb, CD1 lc, CD1 Id, CDS, CEACAM1, CRT AM, cytokine receptor, DAP-10, DNAM1 (CD226), Fc gamma receptor, GADS, GITR, HVEM (EIGHTR), IA4, ICAM-1, ICAM-1, Ig alpha (CD79a), IE-2R beta, IE-2R gamma, IE-7R alpha, inducible T cell costimulator (ICOS), integrins, ITGA4, ITGA4, ITGA6, IT GAD, ITGAE, ITGAE, IT GAM, ITGAX, ITGB2, ITGB7, ITGB1, KIRDS2, EAT, LFA-1, LFA-1, a ligand that specifically binds with CD83, LIGHT, LIGHT, LTBR, Ly9 (CD229), lymphocyte function-associated antigen-1 (LFA-1; CDl-la/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Lyl08), SLAMF7, SLP-76, TNF receptor proteins, TNFR2, TNFSFi4, a Toll ligand receptor, TRANCE/RANKL, VLA1, or VLA-6, or a fragment, truncation, or a combination thereof.

In some embodiments, suitable intracellular signaling domain include, but are not limited to, activating Macrophage/Myeloid cell receptors CSFR1, MYD88, CD14, TIE2, TLR4, CR3, CD64, TREM2, DAP10, DAP12, CD169, DECTIN1, CD206, CD47, CD163, CD36, MARCO, TIM4, MERTK, F4/80, CD91, C1QR, LOX-1, CD68, SRA, BAI-1, ABCA7, CD36, CD31, Lactoferrin, or a fragment, truncation, or combination thereof.

In some embodiments, a receptor tyrosine kinase may be derived from (e.g., comprise) Insulin receptor (InsR), Insulin-like growth factor I receptor (IGF1R), Insulin receptor-related receptor (IRR), platelet derived growth factor receptor alpha (PDGFRa), platelet derived growth factor receptor beta (PDGFRfi). KIT proto-oncogene receptor tyrosine kinase (Kit), colony stimulating factor 1 receptor (CSFR), fms related tyrosine kinase 3 (FLT3), fms related tyrosine kinase 1 (VEGFR-1), kinase insert domain receptor (VEGFR-2), fms related tyrosine kinase 4 (VEGFR-3), fibroblast growth factor receptor 1 (FGFR1), fibroblast growth factor receptor 2 (FGFR2), fibroblast growth factor receptor 3 (FGFR3), fibroblast growth factor receptor 4 (FGFR4), protein tyrosine kinase 7 (CCK4), neurotrophic receptor tyrosine kinase 1 (trkA), neurotrophic receptor tyrosine kinase 2 (trkB), neurotrophic receptor tyrosine kinase 3 (trkC), receptor tyrosine kinase like orphan receptor 1 (ROR1), receptor tyrosine kinase like orphan receptor 2 (ROR2), muscle associated receptor tyrosine kinase (MuSK), MET proto-oncogene, receptor tyrosine kinase (MET), macrophage stimulating 1 receptor (Ron), AXL receptor tyrosine kinase (Axl), TYR03 protein tyrosine kinase (Tyro3), MER proto-oncogene, tyrosine kinase (Mer), tyrosine kinase with immunoglobulin like and EGF like domains 1 (TIE1), TEK receptor tyrosine kinase (TIE2), EPH receptor A1 (EphAl), EPH receptor A2 (EphA2), (EPH receptor A3) EphA3, EPH receptor A4 (EphA4), EPH receptor A5 (EphA5), EPH receptor A6 (EphA6), EPH receptor A7 (EphA7), EPH receptor A8 (EphA8), EPH receptor A10 (EphAlO), EPH receptor B1 (EphBl), EPH receptor B2 (EphB2), EPH receptor B3 (EphB3), EPH receptor B4 (EphB4), EPH receptor B6 (EphB6), ret proto oncogene (Ret), receptor-like tyrosine kinase (RYK), discoidin domain receptor tyrosine kinase 1 (DDR1), discoidin domain receptor tyrosine kinase 2 (DDR2), c-ros oncogene 1, receptor tyrosine kinase (ROS), apoptosis associated tyrosine kinase (Lmrl), lemur tyrosine kinase 2 (Lmr2), lemur tyrosine kinase 3 (Lmr3), leukocyte receptor tyrosine kinase (LTK), ALK receptor tyrosine kinase (ALK), or serine/threonine/tyrosine kinase 1 (STYK1).

iv. Costimulatory Domain

In certain embodiments, the CAR comprises a costimulatory domain. In some embodiments, the costimulatory domain comprises 4-1BB (CD137), CD28, or both, and/or an intracellular T cell signaling domain. In a preferred embodiment, the costimulatory domain is human CD28, human 4-1BB, or both, and the intracellular T cell signaling domain is human CD3 zeta (ζ). 4-1BB, CD28, CD3 zeta may comprise less than the whole 4-1BB, CD28 or CD3 zeta, respectively. Chimeric antigen receptors may incorporate costimulatory (signaling) domains to increase their potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Amur. Rev. Pharmacol. Toxicol. 56:59-83 (2016).

In some embodiments, a costimulatory domain comprises the amino acid sequence of SEQ ID NO: 4041 (KRGRKKLLYIFKQPFMRPVQTTQEEDGCSCRFPEEEEGGCEL) or 4042 (QVQLVQSGAEVEKPGASVKVSCKASGYTFTDYYMHWVRQAPGQGLEWMGWINP NSGGTNYAQKFQGRVTMTRDTSISTAYMEL SRLRSDDTAVYYCASGWDFDYWGQG TLVTVSSGGGGSGGGGSGGGGSGGGGSDIVMTQSPSSLSASVGDRVTITCRASQSIRY YLSWYQQKPGKAPKLLIYTASILQNGVPSRFSGSGSGTDFTLTISSLQPEDFATYYCL QTYTTPDFGPGTKVEIK. See, e.g., PCT Application No. US2022/33091 (WO202261490), which is incorporated herein by reference in its entirety.

v. Intracellular Signaling Domain

The intracellular (signaling) domain of the engineered T cells disclosed herein may provide signaling to an activating domain, which then activates at least one of the normal effector functions of the immune cell. Effector function of a T cell, for example, may be cytolytic activity or helper activity including the secretion of cytokines.

In some embodiments, suitable intracellular signaling domain comprise, but are not limited to 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD 19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD1 la, CD1 lb, CD1 lc, CD1 id, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, LFA-1, ligand that specifically binds with CD83, LIGHT, LTBR, Ly9 (CD229), Lyl08, lymphocyte function-associated antigen-1 (LFA-1; CDl-la/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A), SLAMF7, SLP-76, TNF receptor proteins, TNFR2, TNFSFi4, a Toll ligand receptor, TRANCE/RANKL, VLA1, or VLA-6, or a fragment, truncation, or a combination thereof.

CD3 is an element of the T cell receptor on native T cells, and has been shown to be an important intracellular activating element in CARs. In some embodiments, the CD3 is CD3 zeta. In some embodiments, the activating domain comprises an amino acid sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to the sequence of SEQ ID NO: 4043 (RVKFSRSADAPAYKQGQNQLYNELNLGRREEYDVLDKRRGRDPEMGGKPRRKNP QEGLYNELQKDKMAEAYSEIGMKGERRRGKGHDGLYQGLSTATKDTYDALHMQA LPPR). See, e.g., PCT Application No. US2022/33091, which is incorporated herein by reference in its entirety.

b. T-Cell Receptors (TCR)

In some embodiments, a provided polynucleotide (e.g., a DNA template, a linear precursor RNA polynucleotide, or a circular RNA polynucleotide) encodes a T-cell receptor. TCRs are described using the International Immunogenetics (IMGT) TCR nomenclature, and links to the IMGT public database of TCR sequences. Native alpha-beta heterodimeric TCRs have an alpha chain and a beta chain. Broadly, each chain may comprise variable, joining and constant regions, and the beta chain also usually contains a short diversity region between the variable and joining regions, but this diversity region is often considered as part of the joining region. Each variable region may comprise three CDRs (Complementarity Determining Regions) embedded in a framework sequence, one being the hypervariable region named CDR3. There are several types of alpha chain variable (Vα) regions and several types of beta chain variable (Vβ) regions distinguished by their framework, CDR1 and CDR2 sequences, and by a partly defined CDR3 sequence. The Va types are referred to in IMGT nomenclature by a unique TRAV number. Thus “TRAV21” defines a TCR Vα region having unique framework and CDR1 and CDR2 sequences, and a CDR3 sequence which is partly defined by an amino acid sequence which is preserved from TCR to TCR but which also includes an amino acid sequence which varies from TCR to TCR. In the same way, “TRBV5-1” defines a TCR V3 region having unique framework and CDR1 and CDR2 sequences, but with only a partly defined CDR3 sequence.

The joining regions of the TCR are similarly defined by the unique IMGT TRAJ and TRBJ nomenclature, and the constant regions by the IMGT TRAC and TRBC nomenclature.

The beta chain diversity region is referred to in IMGT nomenclature by the abbreviation TRBD, and, as mentioned, the concatenated TRBD/TRBJ regions are often considered together as the joining region.

The unique sequences defined by the IMGT nomenclature are widely known and accessible to those working in the TCR field. For example, they can be found in the IMGT public database. The “T cell Receptor Factsbook”, (2001) LeFranc and LeFranc, Academic Press, ISBN 0-12-441352-8 also discloses sequences defined by the IMGT nomenclature, but because of its publication date and consequent time-lag, the information therein sometimes needs to be confirmed by reference to the IMGT database.

Native TCRs exist in heterodimeric αβ or γδ forms. However, recombinant TCRs consisting of αα or ββhomodimers have previously been shown to bind to peptide MHC molecules. Therefore, the TCR of the present disclosure may be a heterodimeric ap TCR or may be an aa or pp homodimeric TCR.

For use in adoptive therapy, an ap heterodimeric TCR may, for example, be transfected as full length chains having both cytoplasmic and transmembrane domains. In certain embodiments TCRs of the present disclosure may have an introduced disulfide bond between residues of the respective constant domains, as described, for example, in WO 2006/000830.

TCRs of the present disclosure, particularly alpha-beta heterodimeric TCRs, may comprise an alpha chain TRAC constant domain sequence and/or a beta chain TRBC1 or TRBC2 constant domain sequence. The alpha and beta chain constant domain sequences may be mutated by truncation or substitution to delete the native disulfide bond between Cys4 of exon 2 of TRAC and Cys2 of exon 2 of TRBC1 or TRBC2. The alpha and/or beta chain constant domain sequence(s) may also be mutated by substitution of cysteine residues for Thr 48 of TRAC and Ser 57 of TRBC1 or TRBC2, the said cysteines forming a disulfide bond between the alpha and beta constant domains of the TCR.

Binding affinity (inversely proportional to the equilibrium constant K_{D}) and binding half-life (expressed as T ½) can be determined by any appropriate method. It will be appreciated that doubling the affinity of a TCR results in halving the K_{D}. T¹² is calculated as ln 2 divided by the off-rate (k_{off}). So doubling of T½ results in a halving in k_{off}. K_{D} and k_{off} values for TCRs are usually measured for soluble forms of the TCR, i.e. those forms which are truncated to remove cytoplasmic and transmembrane domain residues. Therefore it is to be understood that a given TCR has an improved binding affinity for, and/or a binding half-life for the parental TCR if a soluble form of that TCR has the said characteristics. Preferably the binding affinity or binding half-life of a given TCR is measured several times, for example, 3 or more times, using the same assay protocol, and an average of the results is taken.

Since the TCRs of the present disclosure have utility in adoptive therapy, the disclosure includes a non-naturally occurring and/or purified and/or or engineered cell, especially a T-cell, presenting a TCR of the present disclosure. There are a number of methods suitable for the transfection of T cells with nucleic acid (such as DNA, cDNA or RNA) encoding the TCRs of the disclosure (see for example Robbins et al., (2008) J Immunol. 180: 6116-6131). T cells expressing the TCRs will be suitable for use in adoptive therapy-based treatment of cancers such as those of the pancreas and liver. As will be known to those skilled in the art, there are a number of suitable methods by which adoptive therapy can be carried out (see for example Rosenberg et al., (2008) Nat Rev Cancer 8(4): 299-308).

As is well-known in the art TCRs of the present disclosure may be subject to post-translational modifications when expressed by transfected cells. Glycosylation is one such modification, which may comprise the covalent attachment of oligosaccharide moieties to defined amino acids in the TCR chain. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (i.e., oligosaccharide type, covalent linkage and total number of attachments) can influence protein function. Therefore, when producing recombinant proteins, controlling glycosylation is often desirable. Glycosylation of transfected TCRs may be controlled by mutations of the transfected gene (Kuball J et al. (2009), J Exp Med 206(2):463-475). Such mutations are also encompassed herein.

A TCR may be specific for an antigen in the group MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (AGE-B4), tyrosinase, brain glycogen phosphorylase, Melan-A, MAGE-C1, MAGE-C2, NY-ESO-1, LAGE-1, SSX-1, SSX-2(HOM-MEL-40), SSX-1, SSX-4, SSX-5, SCP-1, CT-7, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RARa fusion protein, PTPRK, K-ras, N-ras, Triosephosphate isomeras, GnTV, Herv-K-mel, Lage-1, Mage-C2, NA-88, Lage-2, SP17, and TRP2-Int2, (MART-I), gp100 (Pmel 17), TRP-1, TRP-2, MAGE-1, MAGE-3, p15(58), CEA, NY-ESO (LAGE), SCP-1, Hom/Mel-40, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, beta-catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29†BCAA), CA 195, CA 242, CA-50, CAM43, CD68∛KP1, CO-029, FGF-5, G250, Ga733 (EpCAM), HTgp-175, M344, MA-50, MG7-Ag, MOV18, NB†170K, NY-CO-1, RCAS1, SDCCAG16, TA-90 (Mac-2 binding protein†cyclophilin C-associated protein), TAAL6, TAG72, TLP, and TPS.

c. B-Cell Receptors (BCR)

In some embodiments, a provided polynucleotide (e.g., a DNA template, a linear precursor RNA polynucleotide, or a circular RNA polynucleotide) encodes one or more B-cell receptors (BCRs). BCRs (or B-cell antigen receptors) are immunoglobulin molecules that form a type I transmembrane protein on the surface of a B cell. A BCR is capable of transmitting activatory signal into a B cell following recognition of a specific antigen. Prior to binding of a B cell to an antigen, the BCR will remain in an unstimulated or “resting” stage. Binding of an antigen to a BCR leads to signaling that initiates a humoral immune response.

A BCR is expressed by mature B cells. These B cells work with immunoglobulins (Igs) in recognizing and tagging pathogens. The typical BCR comprises a membrane-bound immunoglobulin (e.g., mIgA, mIgD, mIgE, mIgG, and mIgM), along with associated and Igα/Igβ (CD79a/CD79b) heterodimers (α/β). These membrane-bound immunoglobulins are tetramers consisting of two identical heavy and two light chains. Within the BCR, the membrane bound immunoglobulins is capable of responding to antigen binding by signal transmission across the plasma membrane leading to B cell activation and consequently clonal expansion and specific antibody production (Friess M et al. (2018), Front. Immunol. 2947(9)). The Igα/Igβ heterodimers is responsible for transducing signals to the cell interior.

A Igα/Igβ heterodimer signaling relies on the presence of immunoreceptor tyrosine-based activation motifs (ITAMs) located on each of the cytosolic tails of the heterodimers. ITAMs comprise two tyrosine residues separated by 9-12 amino acids (e.g., tyrosine, leucine, and/or valine). Upon binding of an antigen, the tyrosine of the BCR's ITAMs become phosphorylated by Src-family tyrosine kinases Blk, Fyn, or Lyn (Janeway C et al., Immunobiology: The Immune System in Health and Disease (Garland Science, 5th ed. 2001)).

d. Other Chimeric Proteins

In addition to the chimeric proteins provided above, a provided polynucleotide (e.g., a DNA template, a linear precursor RNA polynucleotide, or a circular RNA polynucleotide) may encode for a various number of other chimeric proteins available in the art. The chimeric proteins may include recombinant fusion proteins, chimeric mutant protein, or other fusion proteins.

### B. Immune Modulatory Ligands and Cytokines

In some embodiments, a provided polynucleotide (e.g., a DNA template, a linear precursor RNA polynucleotide, or a circular RNA polynucleotide) encodes for an immune modulatory ligand. In certain embodiments, the immune modulatory ligand may be immunostimulatory; while in other embodiments, the immune modulatory ligand may be immunosuppressive.

In some embodiments, the circular RNA polynucleotide encodes for a cytokine or a functional fragment thereof, including but not limited to interferons, chemokines, interleukins, growth factors, and other cytokines known in the art. In some embodiments, the cytokine comprises a chemokine, interferon, interleukin, lymphokine, and/or tumor necrosis factor. Chemokines are chemotactic cytokine produced by a variety of cell types in acute and chronic inflammation that mobilizes and activates white blood cells. An interferon comprises a family of secreted α-helical cytokines induced in response to specific extracellular molecules through stimulation of TLRs (Borden, Molecular Basis of Cancer (Fourth Edition) 2015). Interleukins are cytokines expressed by leukocytes.

Descriptions and/or amino acid sequences of IL-2, IL-7, IL-10, IL-12, IL-15, IL-18, IL-27β, IFNγ, and/or TGFβ1 are provided herein and at the www.uniprot.org database at accession numbers: P60568 (IL-2), P29459 (IL-12A), P29460 (IL-12B), P13232 (IL-7), P22301 (IL-10), P40933 (IL-15), Q14116 (IL-18), Q14213 (IL-270), P01579 (IFNγ), and/or P01137 (TGFβ1).

### C. Transcription Factors

Regulatory T cells (Treg) are important in maintaining homeostasis, controlling the magnitude and duration of the inflammatory response, and in preventing autoimmune and allergic responses.

In general, Tregs are thought to be mainly involved in suppressing immune responses, functioning in part as a “self-check” for the immune system to prevent excessive reactions. In particular, Tregs are involved in maintaining tolerance to self-antigens, harmless agents such as pollen or food, and abrogating autoimmune disease.

Tregs are found throughout the body including, without limitation, the gut, skin, lung, and liver. Additionally, Treg cells may also be found in certain compartments of the body that are not directly exposed to the external environment such as the spleen, lymph nodes, and even adipose tissue. Each of these Treg cell populations is known or suspected to have one or more unique features and additional information may be found in Lehtimaki and Lahesmaa, Regulatory T cells control immune responses through their non-redundant tissue specific features, 2013, FRONTIERS IN IMMUNOL., 4(294): 1-10, the disclosure of which is hereby incorporated in its entirety.

Typically, Tregs are known to require TGF-β and TL-2 for proper activation and development. Tregs, expressing abundant amounts of the IL-2 receptor (IL-2R), are reliant on IL-2 produced by activated T cells. Tregs are known to produce both IL-10 and TGF-β, both potent immune suppressive cytokines. Additionally, Tregs are known to inhibit the ability of antigen presenting cells (APCs) to stimulate T cells. One proposed mechanism for APC inhibition is via CTLA-4, which is expressed by Foxp3+ Tregs. It is thought that CTLA-4 may bind to B7 molecules on APCs and either block these molecules or remove them by causing internalization resulting in reduced availability of B7 and an inability to provide adequate co-stimulation for immune responses. Additional discussion regarding the origin, differentiation and function of Tregs may be found in Dhamne et al., Peripheral and thymic Foxp3+ regulatory T cells in search of origin, distinction, and function, 2013, Frontiers in Immunol., 4 (253): 1-11, the disclosure of which is hereby incorporated in its entirety.

### D. Checkpoint Inhibitors & Agonists

In some embodiments, a provided polynucleotide (e.g., a DNA template, a linear precursor RNA polynucleotide, or a circular RNA polynucleotide) encodes one or more checkpoint inhibitors or agonists.

In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of IDO1, CTLA4, PD-1, LAG3, PD-L1, TIM3, or combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the immune checkpoint inhibitor is an inhibitor of IDOi.

As described herein, at least in one aspect, the disclosure encompasses the use of immune checkpoint antagonists. Such immune checkpoint antagonists include antagonists of immune checkpoint molecules such as Cytotoxic T-Lymphocyte Antigen 4 (CTLA-4), Programmed Cell Death Protein 1 (PD-1), Programmed Death-Ligand 1 (PDL-1), Lymphocyte-activation gene 3 (LAG-3), and T-cell immunoglobulin and mucin domain 3 (TIM-3). An antagonist of CTLA-4, PD-1, PDL-1, LAG-3, or TIM-3 interferes with CTLA-4, PD-1, PDL-1, LAG-3, or TIM-3 function, respectively. Such antagonists of CTLA-4, PD-1, PDL-1, LAG-3, and TIM-3 can include antibodies which specifically bind to CTLA-4, PD-1, PDL-1, LAG-3, and TIM-3, respectively and inhibit and/or block biological activity and function.

### E. Others

In some embodiments, the payload encoded within one or more of the coding elements is a hormone, FC fusion protein, anticoagulant, blood clotting factor, protein associated with deficiencies and genetic disease, a chaperone protein, an antimicrobial protein, an enzyme (e.g., metabolic enzyme), a structural protein (e.g., a channel or nuclear pore protein), protein variant, small molecule, antibody, nanobody, an engineered non-body antibody, or a combination thereof.

### 4. Production of Polynucleotides

### A. Precursor RNA Preparation

The DNA templates provided herein can be made using standard techniques of molecular biology. For example, the various elements of the vectors provided herein can be obtained using recombinant methods, such as by screening cDNA and genomic libraries from cells, or by deriving the polynucleotides from a DNA template known to include the same.

The various elements of the DNA template provided herein can also be produced synthetically, rather than cloned, based on the known sequences. The complete sequence can be assembled from overlapping oligonucleotides prepared by standard methods and assembled into the complete sequence. See, e.g., Edge, Nature (1981) 292:756; Nambair et al., Science (1984) 223: 1299; and Jay et al., J. Biol. Chem. (1984) 259:631 1.

Thus, particular nucleotide sequences can be obtained from DNA template harboring the desired sequences or synthesized completely, or in part, using various oligonucleotide synthesis techniques known in the art, such as site-directed mutagenesis and polymerase chain reaction (PCR) techniques where appropriate. One method of obtaining nucleotide sequences encoding the desired DNA template elements is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al., Proc. Natl. Acad. Sci. USA (1991) 88:4084-4088. Additionally, oligonucleotide-directed synthesis (Jones et al., Nature (1986) 54:75-82), oligonucleotide directed mutagenesis of preexisting nucleotide regions (Riechmann et al., Nature (1988) 332:323-327 and Verhoeyen et al., Science (1988) 239: 1534-1536), and enzymatic filling-in of gapped oligonucleotides using T4 DNA polymerase (Queen et al., Proc. Natl. Acad. Sci. USA (1989) 86: 10029-10033) can be used.

Transcription of a DNA template (e.g., comprising a 3′ intron element, 3′ exon element, an intervening region or core functional element including an IRES and expression sequence, a 5′ exon element, and a 5′ intron element) results in formation of a precursor linear RNA polynucleotide capable of circularizing. In some embodiments, this DNA template comprises a vector, PCR product, plasmid, minicircle DNA, cosmid, artificial chromosome, complementary DNA (cDNA), extrachromosomal DNA (ecDNA), or a fragment therein. In certain embodiments, the minicircle DNA may be linearized or non-linearized. In certain embodiments, the plasmid may be linearized or non-linearized. In some embodiments, the DNA template may be single-stranded. In other embodiments, the DNA template may be double-stranded. In some embodiments, the DNA template comprises in whole or in part from a viral, bacterial or eukaryotic vector. In some embodiments, the polynucleotide of the present disclosure is an expression vector.

The precursor RNA provided herein can be generated by incubating a DNA template provided herein under conditions permissive of transcription of the precursor RNA encoded by the DNA template. For example, in some embodiments a precursor RNA is synthesized by incubating a DNA template provided herein that comprises an RNA polymerase promoter or promoter segment upstream of its 5′ duplex sequence and/or expression sequences with a compatible RNA polymerase enzyme under conditions permissive of in vitro transcription. In some embodiments, the DNA template is incubated inside of a cell by a bacteriophage RNA polymerase or in the nucleus of a cell by host RNA polymerase II. In some embodiments, the polynucleotide of the present disclosure is an expression vector, wherein the expression vector comprises a polymerase promoter sequence or segment.

In certain embodiments, provided herein is a method of generating precursor RNA by performing in vitro transcription using a DNA template provided herein as a template (e.g., a vector provided herein with an RNA polymerase promoter or promoter segment positioned upstream of the 5′ duplex region).

In some embodiments, the DNA template shares the same sequence as the precursor linear RNA polynucleotide prior to splicing of the precursor linear RNA polynucleotide (e.g., a 3′ intron element, a 3′ exon element, an intervening region core functional element, and a 5′ exon element, a 5′ intron element). In some embodiments, said linear precursor RNA polynucleotide undergoes splicing leading to the removal of the 3′ intron element and 5′ intron element during the process of circularization. In some embodiments, the resulting circular RNA polynucleotide lacks a 3′ intron fragment and a 5′ intron fragment, but maintains a 3′ exon fragment, an intervening region or a core functional element, and a 5′ exon element.

In certain embodiments, the resulting precursor RNA can be used to generate circular RNA (e.g., a circular RNA polynucleotide provided herein) by incubating it in the presence of magnesium ions and guanosine nucleotide or nucleoside at a temperature at which RNA circularization occurs (e.g., between 20° C. and 60° C.). Precursor RNA are generally described in PCT Application No. US2022/33091, which is incorporated herein by reference in its entirety.

### B. Circular RNA Preparation

Thus, in certain embodiments provided herein is a method of making circular RNA. In certain embodiments, the method comprises synthesizing precursor RNA by transcription (e.g., run-off transcription) using a vector provided herein (e.g., a 5′ intron element, a 5′ exon element, an intervening region or core functional element, a 3′ exon element, and a 3′ intron element) as a template, and incubating the resulting precursor RNA in the presence of divalent cations (e.g., magnesium ions) and GTP such that it circularizes to form circular RNA. In some embodiments, the precursor RNA disclosed herein is capable of circularizing in the absence of magnesium ions and GTP and/or without the step of incubation with magnesium ions and GTP. In some embodiments, the precursor linear RNA polynucleotide circularizes when incubated in the presence of one or more guanosine nucleotides or nucleoside (e.g., GTP) and a divalent cation (e.g., Mg2+).

In certain embodiments, transcription occurs at a Mg2+ concentration of at least 3 mM of magnesium. In certain embodiments, the transcription occurs at a Mg2+ concentration of no more than 100 mM of magnesium. In certain embodiments, transcription occurs at a Mg2+ concentration of or about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, or 100 mM. In some embodiments, the greater concentration of Mg2+ during transcription of a linear RNA polynucleotide improves circularization and/or splicing as compared to the same linear RNA polynucleotide undergoing transcription at a lower Mg2+ concentration. In some embodiments, the 3′ exon element, 5′ exon element, and/or core functional element in whole or in part promotes the circularization of the precursor linear RNA polynucleotide to form the circular RNA construct provided herein.

In other embodiments, the method comprises ligation. In some embodiments, the method comprises chemical ligation. In some embodiments, the method comprises splint mediated ligation. In some embodiments, the ligation is performed with a T4 ligase using splint DNA.

In some embodiments, the method of preparing a circular RNA comprises providing modified nucleotides or nucleosides to precursor RNA comprising:
a. a 5′ combined accessory element, comprising:
   i. a 3′ intron segment,
   ii. a 3′ exon segment,
b. an intervening region comprising an internal ribosome entry site (IRES) and a noncoding or coding region,
c. a 3′ combined accessory element, comprising:
   i. a 5′ exon segment, and
   ii. a 5′ intron segment.

In some embodiments, the method of preparing a circular RNA comprises providing a first and second linear precursor RNA polynucleotide, wherein the first and second linear precursor RNA polynucleotides are capable of forming a circular RNA (e.g., ligation or permuted introns).

In some embodiments, a first precursor and a second precursor may be ligated to form a circular RNA. In some embodiments, the first precursor and the second precursor each comprise a short adapter sequence at their 5′ and 3′ ends. In some embodiments, the adapter sequences comprise homology arms with splints used for circularization. Splint ligation may be performed in the presence of a DNA splint using a suitable ligase to generate a circular RNA polynucleotide. Ligation methods are known in the art. See, e.g., Wesselhoeft et al., 2019.

In some embodiments, a first precursor and a second precursor may splice to form a circular RNA comprising a sequence of interest, e.g., a coding region. Each of the first precursor and the second precursor comprises at least one fragment of the sequence of interest, e.g., the first precursor comprises the 5′ fragment of the sequence of interest and the second precursor comprises the 3′ fragment of the sequence of interest. In these embodiments, the 5′ fragment of the sequence of interest, the 3′ fragment of the sequence of interest, and two additional fragments of the sequence of interest (Exon 2A, Exon 2B), together form the sequence of interest. In these embodiments, sequence of interest consists of, e.g., in 5′ to 3′ order, the 5′ fragment of the sequence of interest, an exonic fragment of the sequence of interest (Exon 2A), an exonic fragment of the sequence of interest (Exon 2B), and the 3′ fragment of the sequence of interest.

In some embodiments, the first precursor comprises the following:
(a) two intron fragments (e.g., 3′ intron fragment of a first intron (Intron 1) and a 5′ intron fragment of a second intron (Intron 2)),
(b) a translation initiation element (e.g., IRES),
(c) a 5′ fragment of the sequence of interest, and
(d) two exon fragments that correspond with the intron fragments (e.g., Exon 1B and Exon 2A).

In some embodiments, one exon fragment (e.g., Exon 2A) is a part of a sequence of interest, for example in the coding or noncoding region. The coding region is scanned for sequences that are homologous to this exon (Exon 2A) fragment, thereby allowing splicing to occur without altering the resulting coding sequence in the circular RNA.

In some embodiments, the second precursor comprises the following:
(a) two intron fragments that correspond with those in the first precursor (e.g., 3′ intron fragment of the second intron (Intron 2) and the 5′ intron fragment of the first intron (Intron 1)), and
(b) two exon fragments that correspond with those on the first precursor (e.g., Exon 2B, which corresponds to the 5′ fragment of the 3′ fragment of the sequence of interest (e.g., coding region) and Exon 1A), and
(c) the 3′ fragment of the sequence of interest.

In some embodiments, the coding sequence is scanned for regions that are homologous to this exon (Exon 2B) fragment, thereby allowing splicing to occur without altering the resulting coding sequence in the circular RNA. The first precursor and the second precursor may be incubated together to facilitate splicing between the first precursor and the second precursor in order to generate a circular RNA polynucleotide, which comprises specific modified regions and specific unmodified regions.

In some embodiments, a first precursor comprises an optional first external homology region (Arm 1A), a first intron fragment (3′ intron fragment of a first intron (Intron 1)), a first exon fragment (Exon 1), an optional internal homology region, an optional spacer, a translation initiation element (e.g., IRES), the 5′ fragment of the sequence of interest (e.g., coding region), a second exon fragment (Exon 2A), a second intron fragment (5′ intron fragment of a second intron (Intron 2)), and an optional second external homology region (Arm 2A). In these embodiments, the second precursor comprises an optional first external homology region (Arm 2B), a first intron fragment (3′ intron fragment of the second intron (Intron 2)), a first exon fragment (Exon 2B corresponding to the 5′ fragment of the 3′ fragment of the sequence of interest (e.g., coding region)), the 3′ fragment of the sequence of interest (e.g., coding region), an optional spacer, an optional internal homology region, a second exon fragment (Exon 1A), a second intron fragment (5′ fragment of the first intron (Intron 1)), and an optional second external homology region (Arm 1).

In some embodiments, either the first precursor or the second precursor comprises a monotron.

In some embodiments, the first precursor comprises an optional first external homology region (Arm 1A), a first intron fragment (3′ intron fragment of a first intron (Intron 1)), a first exon fragment (Exon 1B), an optional internal homology region, an optional spacer, a translation initiation element (e.g., IRES), the 5′ fragment of the sequence of interest (e.g., coding region), a second exon fragment (Exon 2A), a terminal element corresponding to a monotron sequence, and an optional second external homology region (Arm 2A). In these embodiments, the second precursor comprises an optional external homology region (Arm 2B), the monotron sequence via Intron 2, a first exon fragment (Exon 2B, which corresponds to the 5′ fragment of the 3′ fragment of the sequence of interest (e.g., coding region)), the 3′ fragment of the sequence of interest (e.g., coding region), an optional spacer, an optional internal homology region, a second exon fragment (Exon 1A), an intron fragment (5′ intron fragment of Intron 2), and an optional second external homology region (Arm 1B).

In some embodiments, the first precursor comprises an optional first external homology region (Arm 1A), a monotron sequence via Intron 1, a first exon fragment (Exon 1B), an optional internal homology region, an optional spacer, a translation initiation element (e.g., IRES), the 5′ fragment of the sequence of interest (e.g., coding region), a second exon fragment (Exon 2A), an intron fragment (5′ intron fragment of Intron 2), and an optional second external homology region. In these embodiments, the second precursor comprises an optional external homology region (Arm 2B), an intron fragment (3′ intron fragment of Intron 2), a first exon fragment (Exon 2B, which corresponds to the 5′ fragment of the 3′ fragment of the sequence of interest (e.g., coding region)), a 3′ fragment of the sequence of interest (e.g., coding region), an optional spacer, an optional internal homology region, a second exon fragment (Exon 1A), a terminal element corresponding to the monotron sequence, and an optional second external homology region (Arm 1).

In some embodiments, each of the first precursor and the second precursor comprises a monotron. In some embodiments, the first precursor comprises an optional first external homology region (Arm 1A), a first monotron sequence via Intron 1, a first exon fragment (Exon 1B), an optional internal homology region, an optional spacer, a translation initiation element (e.g., IRES), the 5′ fragment of the sequence of interest, a second exon fragment (Exon 2A), a terminal element corresponding to a second monotron sequence via Intron 2, and an optional second external homology region (Arm 2A). In these embodiments, the second precursor comprises an optional external homology region (Arm 2B), a second monotron sequence via Intron 2, a first exon fragment (Exon 2B), the 3′ fragment of the sequence of interest (e.g., coding region), an optional spacer, an optional internal homology region, a second exon fragment (Exon IA), a terminal element corresponding to the first monotron sequence via Intron 1, and an optional second external homology region (Arm 1B).

In some embodiments, provided herein are circular RNA that do not comprise modified nucleotides and/or modified nucleosides. Also provided herein are modified circular RNA (i.e., comprising at least one modified nucleotide and/or modified nucleoside) prepared from the methods described herein. In some embodiments, the modified circular RNA affects immunogenicity, circularization, and/or translation as compared to circular RNA prepared with RNA precursor polynucleotides that comprise no nucleotide or nucleoside modifications.

It has been discovered that circular RNA has reduced immunogenicity relative to a corresponding mRNA, at least partially because the mRNA contains an immunogenic 5′ cap. When transcribing a DNA vector from certain promoters (e.g., a T7 promoter, SP6 promoter, or a fragment thereof) to produce a precursor RNA, it is understood that the 5′ end of the precursor RNA is G. To reduce the immunogenicity of a circular RNA composition that contains a low level of contaminant linear mRNA, an excess of GMP relative to GTP can be provided during transcription such that most transcripts contain a 5′ GMP, which cannot be capped. Therefore, in some embodiments, transcription is carried out in the presence of an excess of GMP. In some embodiments, transcription is carried out where the ratio of GMP concentration to GTP concentration is within the range of about 3:1 to about 15:1, for example, about 3:1 to about 10:1, about 3:1 to about 5:1, about 3:1, about 4:1, or about 5:1.

In some embodiments, a composition comprising circular RNA has been purified. Circular RNA may be purified by any known method commonly used in the art, such as column chromatography, gel filtration chromatography, and size exclusion chromatography. In some embodiments, purification comprises one or more of the following steps: phosphatase treatment, HPLC size exclusion purification, and RNase R digestion. In some embodiments, purification comprises the following steps in order: RNase R digestion, phosphatase treatment, and HPLC size exclusion purification. In some embodiments, purification comprises reverse phase HPLC. In some embodiments, a purified composition contains less double stranded RNA, DNA splints, triphosphorylated RNA, phosphatase proteins, protein ligases, capping enzymes and/or nicked RNA than unpurified RNA. In some embodiments, purification of circular RNA comprises an affinity-purification or negative selection method described herein. In some embodiments, purification of circular RNA comprises separation of linear RNA from circular RNA using oligonucleotides that are complementary to a sequence in the linear RNA but are not complementary to a sequence in the circular RNA. In some embodiments, a purified composition is less immunogenic than an unpurified composition. In some embodiments, immune cells exposed to a purified composition produce less TNFα, RIG-I, IL-2, IL-6, IFNγ, and/or a type 1 interferon, e.g., IFN-β1, than immune cells exposed to an unpurified composition.

In some embodiments, circular RNA is produced by transcribing a DNA polynucleotide sequence that is complementary to a precursory RNA polynucleotide that is described herein. In certain embodiments, circular RNA provided herein is produced in vitro. In certain embodiments, circular RNA provided herein is produced inside a cell. In some embodiments, the cell selected from, for example, an immune cell, muscle cell, neural cell, epithelial cell and a tumor cell. In some embodiments, precursor RNA is transcribed using a DNA template (e.g., in some embodiments, using a vector provided herein) in the cytoplasm by a bacteriophage RNA polymerase, or in the nucleus by host RNA polymerase II and then circularized.

Exemplary methods of circularization of precursor RNA can be found in, for example, WO2020/237227, which is incorporated by reference herein in its entirety. WO2020/237227, inter alia, describes using the permuted intron exon (PIE) circularization strategy to circularize long precursor RNA. In it, a 1.1 kb sequence containing a full-length encephalomyocarditis virus (EMCV) IRES, a Gaussia luciferase (GLuc) expression sequence, and two short exon fragments of the permuted intron-exon (PIE) construct were inserted between the 3′ and 5′ introns of the permuted group I catalytic intron in the thymidylate synthase (Td) gene of the T4 phage. Precursor RNA was synthesized by run-off transcription. Circularization was attempted by heating the precursor RNA in the presence of magnesium ions and GTP, but splicing products were not obtained. Perfectly complementary 9 nucleotide and 19 nucleotide long homology regions were designed and added at the 5′ and 3′ ends of the precursor RNA. The splicing product was treated with RNase R. Sequencing across the putative splice junction of RNase R-treated splicing reactions revealed ligated exons, and digestion of the RNase R-treated splicing reaction with oligonucleotide-targeted RNase H produced a single band in contrast to two bands yielded by RNase H-digested linear precursor. WO2020/237227 further indicates that a series of spacers was designed and inserted between the 3′ PIE splice site and the IRES. These spacers were designed to either conserve or disrupt secondary structures within intron sequences in the IRES, 3′ PIE splice site, and/or 5′ splice site.

Further methods for preparing circular RNA are described in PCT Application No. US2022/33091, which is incorporated herein by reference in its entirety.

### 5. Transfer Vehicle & Other Delivery Mechanisms

### A. Ionizable Lipids

In certain embodiments, disclosed herein are ionizable lipids that may be used as a component of a transfer vehicle to facilitate or enhance the delivery and release of circular RNA to one or more target cells (e.g., by permeating or fusing with the lipid membranes of such target cells). In certain embodiments, an ionizable lipid comprises one or more cleavable functional groups (e.g., a disulfide) that allow, for example, a hydrophilic functional head-group to dissociate from a lipophilic functional tail-group of the compound (e.g., upon exposure to oxidative, reducing or acidic conditions), thereby facilitating a phase transition in the lipid bilayer of the one or more target cells.

In some embodiments, an ionizable lipid is as described in international patent application PCT/US2020/038678. In some embodiments, an ionizable lipid is a lipid as represented by formula 1 of or as listed in Tables 1 or 2 of U.S. Pat. No. 9,708,628, the content of which is herein incorporated by reference in its entirety. In some embodiments, an ionizable lipid is as described in pages 7-13 of U.S. Pat. No. 9,765,022 or as represented by formula 1 of U.S. Pat. No. 9,765,022, the content of which is herein incorporated by reference in its entirety. In some embodiments, an ionizable lipid is described in pages 12-24 of International Patent Application No. PCT/US2019/016362 or as represented by formula 1 of International Patent Application PCT/US2019/016362, the contents of which are herein incorporated by reference in their entirety. In some embodiments, a lipid or transfer vehicle is a lipid as described in International Patent Application Nos. PCT/US2010/061058, PCT/US2018/058555, PCT/US2018/053569, PCT/US2017/028981, PCT/US2019/025246, PCT/US2019/015913, PCT/US2019/016362, PCT/US2019/016362, US Application Publication Nos. US2019/0314524, US2019/0321489, US2019/0314284, and US2019/0091164, the contents of which are herein incorporated by reference in their entireties. Suitable cationic lipids for use in the compositions and methods herein include those described in international patent publication WO 2010/053572 and/or U.S. patent application Ser. No. 15/809,680, e.g., C12-200. In certain embodiments, the compositions and methods herein employ an ionizable cationic lipid described in WO2013149140 (incorporated herein by reference), such as, e.g., (15Z,18Z)-N,N-dimethyl-6-(9Z,12Z)-octadeca-9,12-dien-1-yl)tetracosa-15,18-dien-1-amine (HGT5000), (15Z,18Z)-N,N-dimethyl-6-((9Z,12Z)-octadeca-9,12-dien-1-yl)tetracosa-4,15,18-trien-1-amine (HGT5001), and (15Z,18Z)-N,N-dimethyl-6-((9Z,12Z)-octadeca-9,12-dien-1-yl)tetracosa-5,15,18-trien-1-amine (HGT5002). In certain embodiments, the compositions and methods herein employ an ionizable cationic lipid described US patent publications 2017/0190661 and 2017/0114010, incorporated herein by reference in their entirety.

In some embodiments, the cationic lipid N-[1-(2,3-dioleyloxy)propyl]-N,N,N-trimethylammonium chloride or “DOTMA” is used. (Felgner et al. (Proc. Nat'l Acad. Sci. 84, 7413 (1987); U.S. Pat. No. 4,897,355). DOTMA can be formulated alone or can be combined with the neutral lipid, dioleoylphosphatidyl-ethanolamine or “DOPE” or other cationic or non-cationic lipids into a transfer vehicle or a lipid nanoparticle, and such liposomes can be used to enhance the delivery of nucleic acids into target cells. Other suitable cationic lipids include, for example, 5-carboxyspermylglycinedioctadecylamide or “DOGS,” 2,3-dioleyloxy-N-[2(spermine-carboxamido)ethyl]-N,N-dimethyl-1-propanaminium or “DOSPA” (Behr et al. Proc. Nat.'l Acad. Sci. 86, 6982 (1989); U.S. Pat. Nos. 5,171,678; 5,334,761), 1,2-Dioleoyl-3-Dimethylammonium-Propane or “DODAP,” 1,2-Dioleoyl-3-Trimethylammonium-Propane or “DOTAP.” Contemplated cationic lipids also include 1,2-distearyloxy-N,N-dimethyl-3-aminopropane or “DSDMA”, 1,2-dioleyloxy-N,N-dimethyl-3-aminopropane or “DODMA,” 1,2-dilinoleyloxy-N,N-dimethyl-3-aminopropane or “DLinDMA,” 1,2-dilinolenyloxy-N,N-dimethyl-3-aminopropane or “DLenDMA,” N-dioleyl-N,N-dimethylammonium chloride or “DODAC,” N,N-distearyl-N,N-dimethylammonium bromide or “DDAB,” N-(1,2-dimyristyloxyprop-3-yl)-N,N-dimethyl-N-hydroxyethyl ammonium bromide or “DMRIE,” 3-dimethylamino-2-(cholest-5-en-3-beta-oxybutan-4-oxy)-1-(cis,cis-9,12-octadecadienoxy)propane or “CLinDMA,” 2-[5′-(cholest-5-en-3-beta-oxy)-3′-oxapentoxy)-3-dimethy 1-1-(cis,cis-9′, 1-2′-octadecadienoxy)propane or “CpLinDMA,” N,N-dimethyl-3,4-dioleyloxybenzylamine or “DMOBA,” 1,2-N,N′-dioleylcarbamyl-3-dimethylaminopropane or “DOcarbDAP,” 2,3-Dilinoleoyloxy-N,N-dimethylpropylamine or “DLinDAP,” 1,2-N,N′-Dilinoleylcarbamyl-3-dimethylaminopropane or “DLincarbDAP,” 1,2-Dilinoleoylcarbamyl-3-dimethylaminopropane or “DLinCDAP,” 2,2-dilinoleyl-4-dimethylaminomethyl-[1,3]-dioxolane or “DLin-K-DMA,” 2,2-dilinoleyl-4-dimethylaminoethyl-[1,3]-dioxolane or “DLin-K-XTC2-DMA,” and 2-(2,2-di((9Z,12Z)-octadeca-9,12-dien-1-yl)-1,3-dioxolan-4-yl)-N,N-dimethylethanamine (DLin-KC2-DMA)) (See, WO 2010/042877; Semple et al., Nature Biotech. 28:172-176 (2010)), or mixtures thereof. (Heyes, J., et al., J Controlled Release 107: 276-287 (2005); Morrissey, D V., et al., Nat. Biotechnol. 23(8): 1003-1007 (2005); PCT Publication WO2005/121348A1).

The use of cholesterol-based cationic lipids to formulate the transfer vehicles (e.g., lipid nanoparticles) is also contemplated herein. Such cholesterol-based cationic lipids can be used, either alone or in combination with other cationic or non-cationic lipids. Suitable cholesterol-based cationic lipids include, for example, GL67, DC-Chol (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE.

In some embodiments, the one or more of the cationic or ionizable lipids provide increased activity of the nucleic acid and improved tolerability of the compositions in vivo.

PCT/US2022/033091 (WO 2022/261490) describes representative cationic lipids of any one of the disclosed embodiments and is incorporated by reference herein in its entirety.

In some embodiments, the cationic lipid (or a pharmaceutically acceptable salt, tautomer, prodrug or stereoisomer thereof) has a structure selected from one of the following that are described in detail on pages 113-118 of WO 2022/261490 and page 113-118 of WO 2023/056033, which are incorporated by reference herein in their entireties:

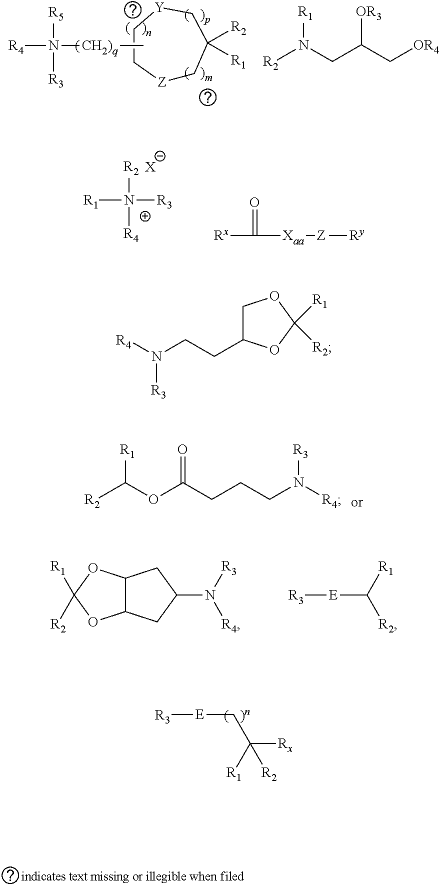

In some embodiments, for example, a cationic lipid of any one of the disclosed embodiments has a structure of Formula I and/or is selected from structure numbers I-1 through I-41, set forth at pages 119-130 and in Table 1 of WO 2022/261490; has a structure of Formula II and/or is selected from structure numbers II-1 through II-46, set forth at pages 130-146 and in Table 2 of WO 2022/261490; has a structure of Formula III and/or is selected from structure numbers III-1 through III-49, set forth at pages 146-157 and in Table 3 of WO 2022/261490; has a structure of Formula IV or V and/or is selected from structure numbers IV-1 through IV-3, set forth at pages 157-174 and in Table 4 of WO 2022/261490; has a structure of Formula VI and/or is selected from structure numbers VI-1 through VI-37, set forth at pages 174-188 and in Table 5 of WO 2022/261490; has a structure of Formula VII and/or is selected from structure numbers VII-1 through VII-11, set forth at pages 188-195 in Table 6 of WO 2022/261490; has a structure of Formula VIII and/or is selected from structure numbers VIII-1 through VII-12, set forth at pages 195-201 and in Table 7 of WO 2022/261490; has a structure of Formula IX and/or is selected from structure numbers IX-1 through IX-18, set forth at pages 201-208 and in Table 8 of WO 2022/261490; has a structure of Formula X and/or is selected from structure numbers X-1 through X-17, set forth at pages 208-213 and in Table 6 of WO 2022/261490; has a structure of Formula XI and/or is structure number XIa or Formula XII and/or is selected from structure numbers XIIA-XIIJ, as described at pages 213-220 in WO 2022/261490. WO 2023/056033 describes similar structures, and is incorporated by reference in its entirety.

In some embodiments, an ionizable lipid is a compound of Formula (1), Formula (1-1), Formula (1-2), Formula (2), Formula (3), Formula (3-1), Formula (3-2), Formula (3-3), Formula (5), or Formula (6), in WO 2022/261490, which is incorporated by reference herein in its entirety. WO 2022/261490 provides exemplary reaction schemes that illustrate an exemplary method to make compounds of Formula (1). WO 2023/056033 describes similar structures, and is incorporated by reference in its entirety.

In some embodiments, the ionizable lipid has a beta-hydroxyl amine head group. In some embodiments, the ionizable lipid has a gamma-hydroxyl amine head group.

In some embodiments, an ionizable lipid of the disclosure is a lipid selected from Table 10a, Table 10b, or Table 10 on pages 235-271—of WO 2022/261490, which is incorporated herein by reference in its entirety. In some embodiments, the ionizable lipid is Lipid 26, 27, 53, 54, 45, 46, 137, 138, 139, 128, or 130 in Table 10a of WO 2022/261490. In some embodiments, an ionizable lipid of the disclosure is Lipid 15 from Table 10b of WO 2022/261490.

In an embodiment, the ionizable lipid is described in US patent publication number US20170210697A1. In an embodiment, the ionizable lipid is described in US patent publication number US20170119904A1.

In some embodiments, the ionizable lipid has one of the structures set forth in Table 11 of WO 2022/261490, which is incorporated herein by reference in its entirety, certain of which are described in international patent application PCT/US2010/061058. In some embodiments, the ionizable lipids may include a lipid selected from Tables 12, 13, 14, or 15a of WO 2022/261490.

In some embodiments, the transfer vehicle comprises Lipid A, Lipid B, Lipid C, and/or Lipid D, described in detail, including methods of synthesis that are known in the art, in WO 2022/261490, WO 2023/056033, and PCT/US2017/028981, which are incorporated herein by reference in their entireties.

Also contemplated are ionizable lipids such as the dialkylamino-based, imidazole-based, and guanidinium-based lipids. See, e.g., PCT/US2010/058457, incorporated herein by reference. For example, certain embodiments are directed to a composition comprising one or more imidazole-based ionizable lipids, for example, the imidazole cholesterol ester or “ICE” lipid, (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, as represented by structure (XIII) of WO 2022/261490 and WO 2023/056033, which are incorporated herein by reference in their entireties. In an embodiment, a transfer vehicle for delivery of circRNA may comprise one or more imidazole-based ionizable lipids, for example, the imidazole cholesterol ester or “ICE” lipid (3S, 10R, 13R, 17R)-10, 13-dimethyl-17-((R)-6-methylheptan-2-yl)-2, 3, 4, 7, 9, 10, 11, 12, 13, 14, 15, 16, 17-tetradecahydro-1H-cyclopenta[a]phenanthren-3-yl 3-(1H-imidazol-4-yl)propanoate, as represented by structure (XIII). In some embodiments, an ionizable lipid is described by US patent publication number 20190314284.

In certain embodiments, the ionizable lipid is described by structure (XIV), structure XVII (referred to herein as “HGT4001”), structure XVIII (referred to herein as “HGT4002”), structure XIX (referred to herein as “HGT4003”), structure XX (referred to herein as “HGT4004”), or structure XXI (referred to herein as “HGT4005”) of WO 2022/261490 and WO 2023/056033, which are incorporated herein by reference in their entireties.

In some embodiments, the ionizable lipid is selected from a lipid with a structure depicted on 390-457 of WO 2022/261490, which is incorporated herein by reference in its entirety.

WO 2023/056033 also describes representative cationic lipids of any one of the disclosed embodiments and is incorporated by reference herein in its entirety. In some embodiments, for example, a cationic lipid of any one of the disclosed embodiments has the structure of Formula (7), (7-1), (7-2), (7-3), (8), (8-1), (8-2), (8-3), (8-4), (9), (10), (11), and/or (12) of WO 2023/056033. In some embodiments, the cationic lipid is selected from a lipid with a structure depicted in any of Tables 10a-10f, Table 11, Tables 12, 13, 14, or 15a of WO 2023/056033. In some embodiments, the ionizable lipid is described by structure (XIV) of WO 2023/056033, and pharmaceutical compositions comprising the compound of structure XIV are envisioned. In some embodiments, the cationic lipid is selected from a lipid with a structure depicted on pages 386-439 of WO 2023/056033.

In some embodiments, the one or more of the cationic or ionizable lipids are represented by Formula (I):

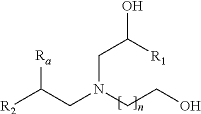

wherein:
   n is an integer between 1 and 4;
   Rₐ is hydrogen or hydroxyl; and
   R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl.

In some embodiments, Rₐ is hydrogen. In some embodiments, Rₐ is hydroxyl.

In some embodiments, the ionizable lipid is represented by Formula (Ia-1), Formula (Ia-2), or Formula (Ia-3):

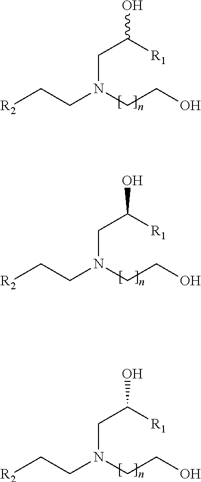

In some embodiments, the ionizable lipid is represented by Formula (Ib-1), Formula (Ib-2), or Formula (Ib-3):

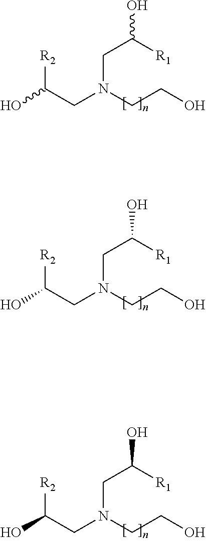

In some embodiments, the ionizable lipid is represented by Formula (Ib-4), Formula (Ib-5), Formula (Ib-6), Formula (Ib-7), Formula (Ib-8), or Formula (Ib-9):

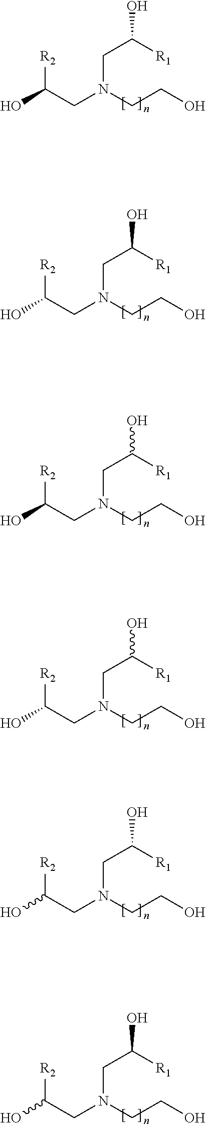

In some embodiments, the one or more of the cationic or ionizable lipids are represented by Formula (I), wherein R₁ and R₂ are each independently selected from:

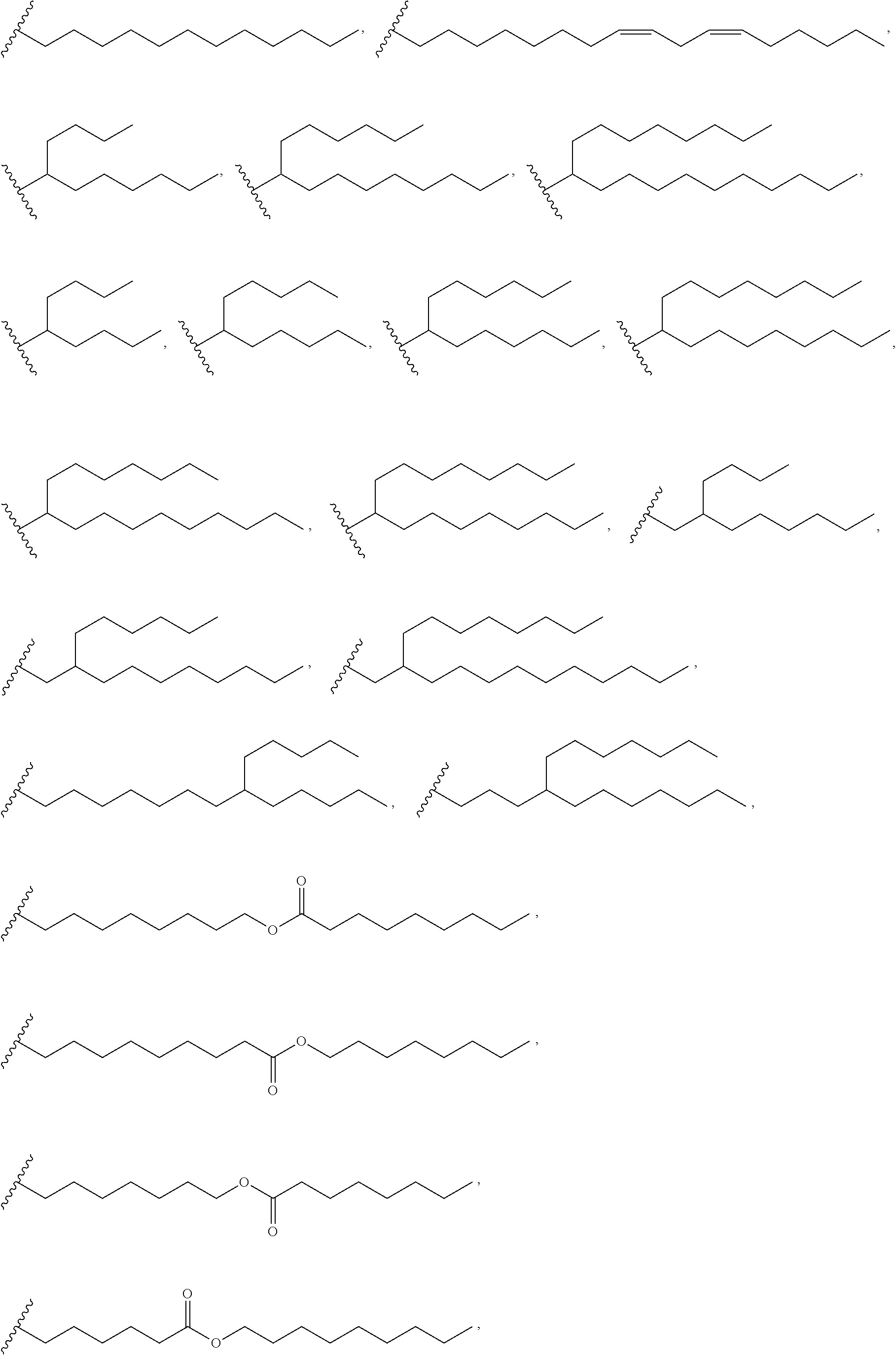

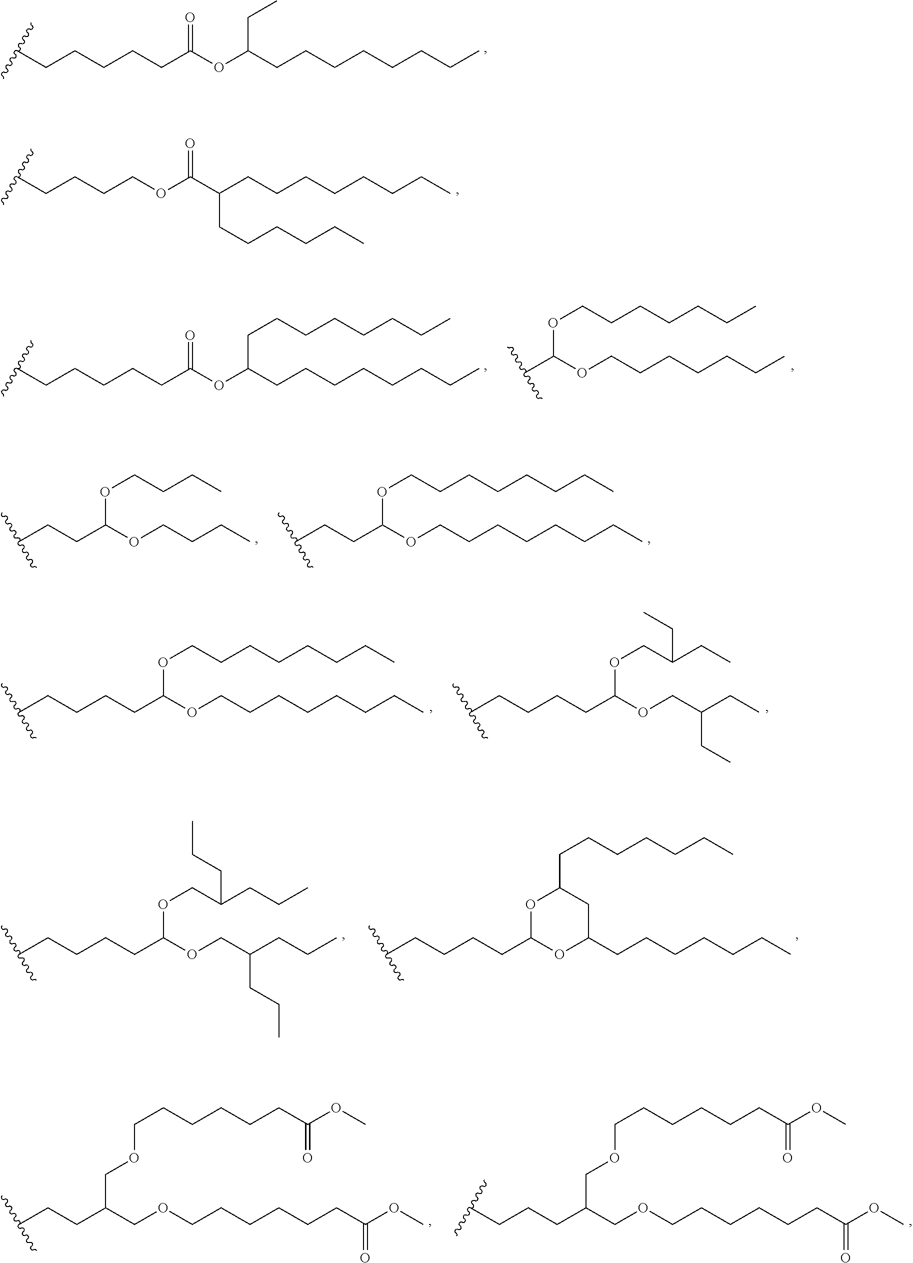

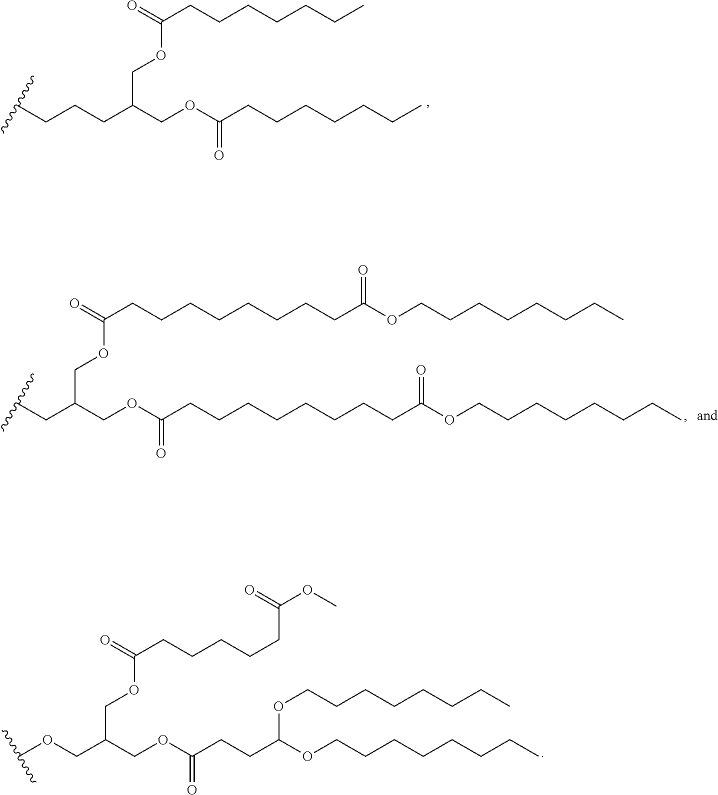 [SMILES: *CCCC(COC(=O)CCCCCCC)COC(=O)CCCCCCC ; *CC(COC(=O)CCCCCCCCC(=O)OCCCCCCCC)COC(=O)CCCCCCCCC(=O)OCCCCCCCC ; *OCC(COC(=O)CCCCCC(=O)OC)COC(=O)CCC(OCCCCCCCC)OCCCCCCCC]

In some embodiments, R₁ and R₂ are the same. In some embodiments, R₁ and R₂ are different.

In various embodiments, the one or more of the cationic or ionizable lipids are represented by Formula (I*):

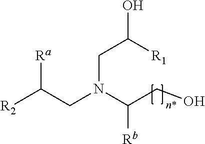

wherein:
n* is an integer between 1 to 7,
R^{a} is hydrogen or hydroxyl,
R^{b} is hydrogen or C₁-C₆ alkyl,
R₁ and R₂ are each independently a linear or branched C₁-C₃₀ alkyl, C₂-C₃₀ alkenyl, or C₁-C₃₀ heteroalkyl, optionally substituted by one or more substituents selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, alkylcarbonyloxy, alkylcarbonate, alkenyloxycarbonyl, alkenylcarbonyloxy, alkenylcarbonate, alkynyloxycarbonyl, alkynylcarbonyloxy, alkynylcarbonate, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl.

In some embodiments, the one or more of the cationic or ionizable lipids are represented by Formula (II):

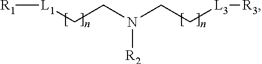

wherein:
each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

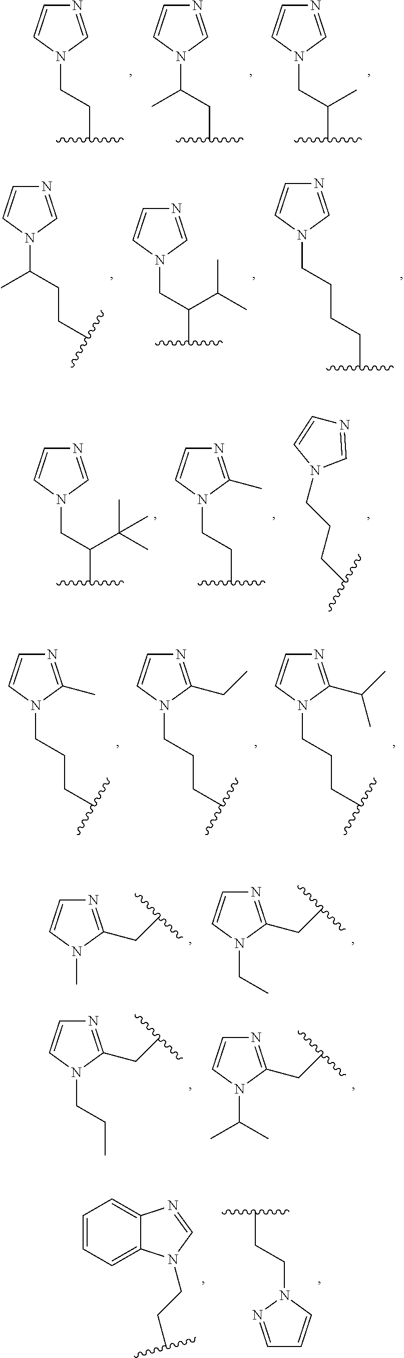

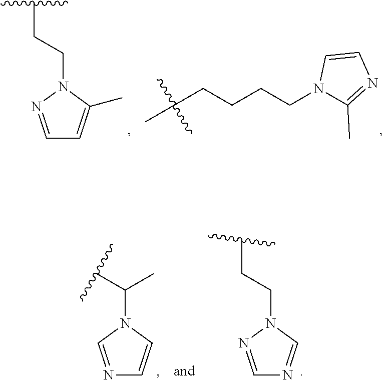

In some embodiments, the ionizable lipid is selected from an ionizable lipid of Formula II, wherein R₁ and R₃ are each independently selected from a group consisting of

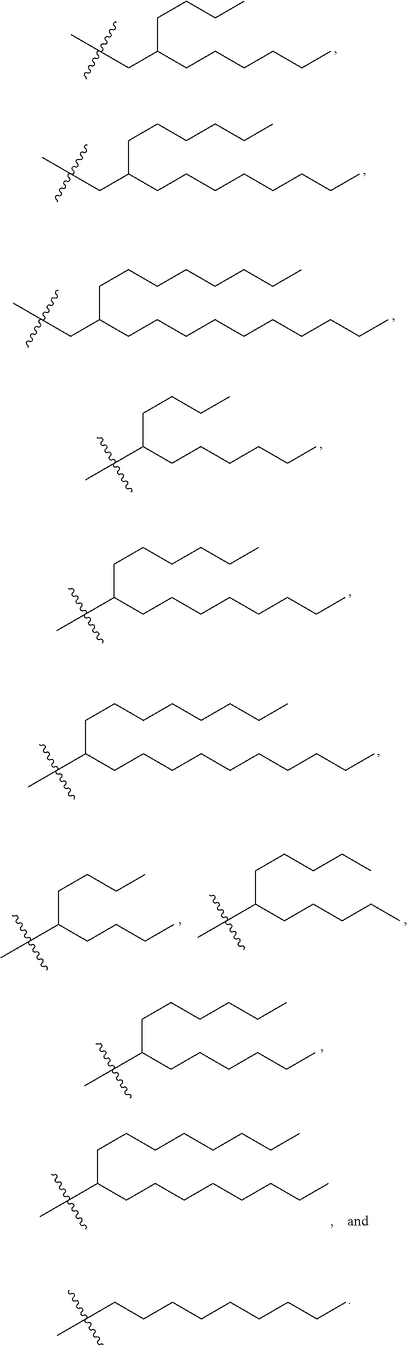

In some embodiments, R₁ and R₃ are the same. In some embodiments, R₁ and R₃ are different.

In some embodiments, the one or more of the cationic or ionizable lipids are represented by Formula (II-1) or Formula (II-2):

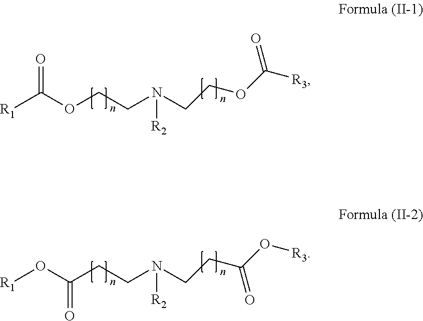

In some embodiments, the ionizable lipid is selected from an ionizable lipid of WO2015/095340 (lipid number 123 of Table 3). In some embodiments, the ionizable lipid is selected from an ionizable lipid of WO2021/021634, WO2020/237227, or WO2019/236673 (lipid numbers 124-127 of Table 3). In some embodiments, the ionizable lipid is selected from an ionizable lipid of WO2021226597 and WO2021113777 (lipid numbers 128-131 of Table 3).

In some embodiments, the transfer vehicle comprises an ionizable lipid selected from an ionizable lipid represented in Table 3. In particular embodiments, where the ionizable lipid is of Formula I, the ionizable lipid is selected from lipid numbers 16, 45, 86, 89, and 90 of Table 3, below. In particular embodiments where the ionizable lipid is an ionizable lipid of Formula II, the ionizable lipid is selected from lipid numbers 128-131 of Table 3, below.

In some embodiments, the one or more of the cationic or ionizable lipids are represented by Formula (III):

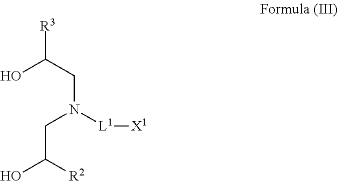

or a pharmaceutically acceptable salt thereof, wherein
L¹ is C₂-C₁₁ alkylene, C₄-C₁₀-alkenylene, or C₄-C₁₀-alkynylene;
X¹ is OR¹, SR¹, or N(R¹)₂, where R¹ is independently H or unsubstituted C₁-C₆ alkyl; and
R² and R³ are each independently C₆-C₃₀-alkyl, C₆-C₃₀-alkenyl, or C₆-C₃₀-alkynyl.

In some embodiments, the one or more of the cationic or ionizable lipids are represented by Formula (III*):

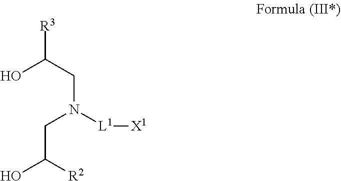

or a pharmaceutically acceptable salt thereof, wherein
L¹ is C₂-C₁₁ alkylene, C₄-C₁₀-alkenylene, or C₄-C₁₀-alkynylene;
X¹ is OR¹, SR¹, or N(R¹)₂, where R¹ is independently H or unsubstituted C₁-C₆ alkyl; and
R₂ and R₃ are each independently a linear or branched C₁-C₃₀ alkyl, C₂-C₃₀ alkenyl, or C₁-C₃₀ heteroalkyl, optionally substituted by one or more substituents selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, alkylcarbonyloxy, alkylcarbonate, alkenyloxycarbonyl, alkenylcarbonyloxy, alkenylcarbonate, alkynyloxycarbonyl, alkynylcarbonyloxy, alkynylcarbonate, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl.

TABLE 3
Exemplary Ionizable Lipid Structures

| Ionizable | |
|---|---|
| lipid | |
| number | Structure |
| 1 | 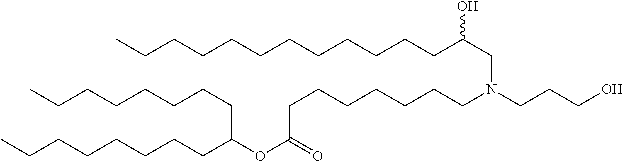 [SMILES: CCCCCCCCCCCCC(O)CN(CCCO)CCCCCCCC(=O)OC(CCCCCCCC)CCCCCCCC] |
| 2 | 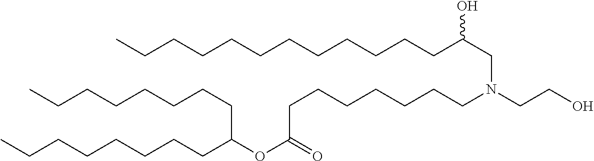 [SMILES: CCCCCCCCCCCCC(O)CN(CCO)CCCCCCCC(=O)OC(CCCCCCCC)CCCCCCCC] |
| 3 | 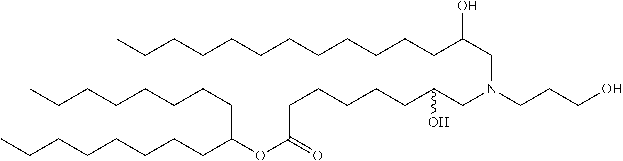 [SMILES: CCCCCCCCCCCCC(O)CN(CCCO)CC(O)CCCCCC(=O)OC(CCCCCCCC)CCCCCCCC] |
| 4 | 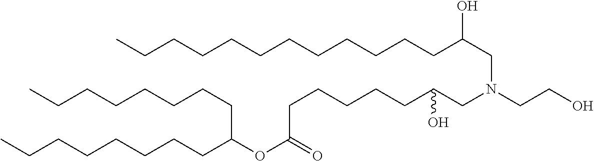 [SMILES: CCCCCCCCCCCCC(O)CN(CCO)CC(O)CCCCCC(=O)OC(CCCCCCCC)CCCCCCCC] |
| 5 | 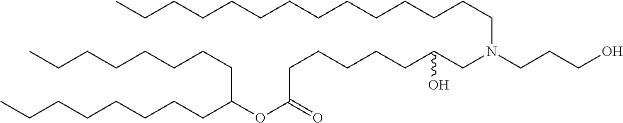 [SMILES: CCCCCCCCCCCCCCN(CCCO)CC(O)CCCCCC(=O)OC(CCCCCCCC)CCCCCCCC] |
| 6 | 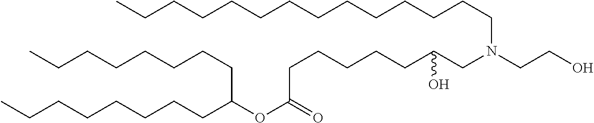 [SMILES: CCCCCCCCCCCCCCN(CCO)CC(O)CCCCCC(=O)OC(CCCCCCCC)CCCCCCCC] |
| 7 | 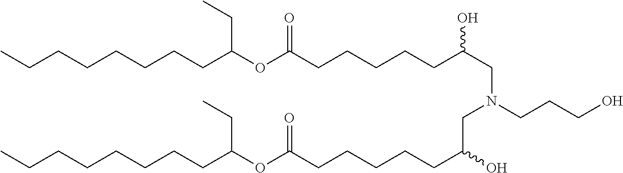 [SMILES: CCCCCCCCC(CC)OC(=O)CCCCCC(O)CN(CCCO)CC(O)CCCCCC(=O)OC(CC)CCCCCCCC] |
| 8 | 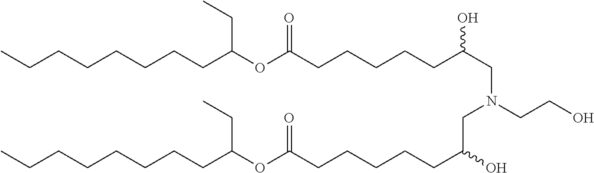 [SMILES: CCCCCCCCC(CC)OC(=O)CCCCCC(O)CN(CCO)CC(O)CCCCCC(=O)OC(CC)CCCCCCCC] |
| 9 | 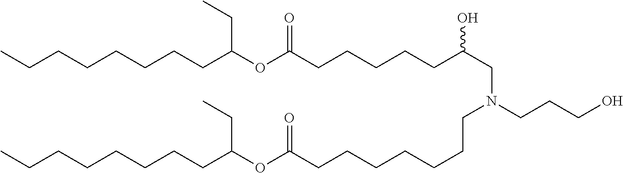 [SMILES: CCCCCCCCC(CC)OC(=O)CCCCCCCN(CCCO)CC(O)CCCCCC(=O)OC(CC)CCCCCCCC] |
| 10 | 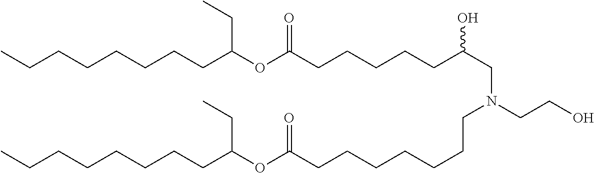 [SMILES: CCCCCCCCC(CC)OC(=O)CCCCCCCN(CCO)CC(O)CCCCCC(=O)OC(CC)CCCCCCCC] |
| 11 | 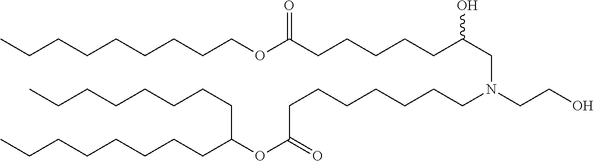 [SMILES: CCCCCCCCCOC(=O)CCCCCC(O)CN(CCO)CCCCCCCC(=O)OC(CCCCCCCC)CCCCCCCC] |
| 12 | 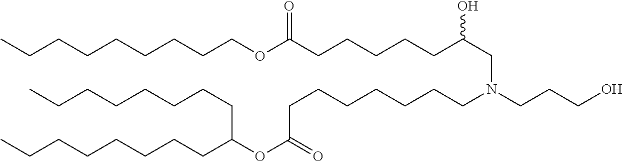 [SMILES: CCCCCCCCCOC(=O)CCCCCC(O)CN(CCCO)CCCCCCCC(=O)OC(CCCCCCCC)CCCCCCCC] |
| 13 | 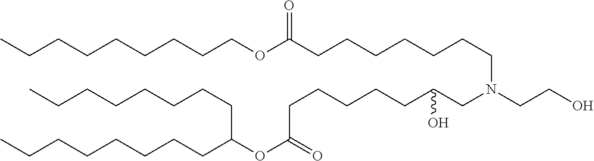 [SMILES: CCCCCCCCCOC(=O)CCCCCCCN(CCO)CC(O)CCCCCC(=O)OC(CCCCCCCC)CCCCCCCC] |
| 14 | 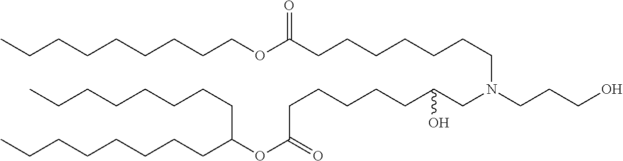 [SMILES: CCCCCCCCCOC(=O)CCCCCCCN(CCCO)CC(O)CCCCCC(=O)OC(CCCCCCCC)CCCCCCCC] |
| 15 | 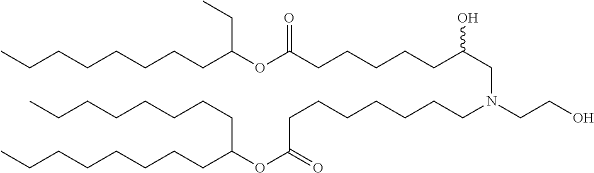 [SMILES: CCCCCCCCC(CC)OC(=O)CCCCCC(O)CN(CCO)CCCCCCCC(=O)OC(CCCCCCCC)CCCCCCCC] |
| 16 | 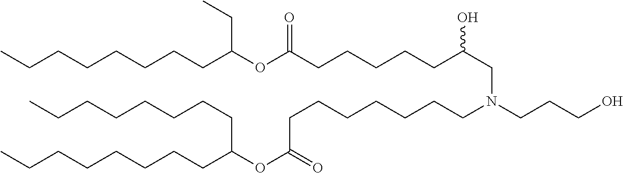 [SMILES: CCCCCCCCC(CC)OC(=O)CCCCCC(O)CN(CCCO)CCCCCCCC(=O)OC(CCCCCCCC)CCCCCCCC] |
| 17 | 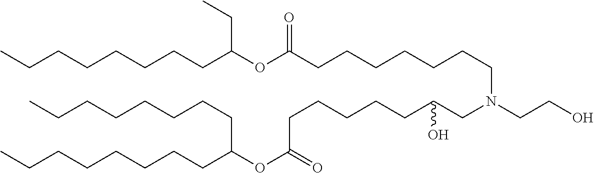 [SMILES: CCCCCCCCC(CC)OC(=O)CCCCCCCN(CCO)CC(O)CCCCCC(=O)OC(CCCCCCCC)CCCCCCCC] |
| 18 | 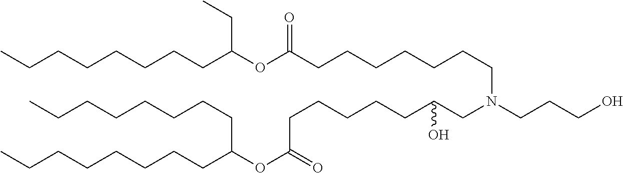 [SMILES: CCCCCCCCC(CC)OC(=O)CCCCCCCN(CCCO)CC(O)CCCCCC(=O)OC(CCCCCCCC)CCCCCCCC] |
| 19 | 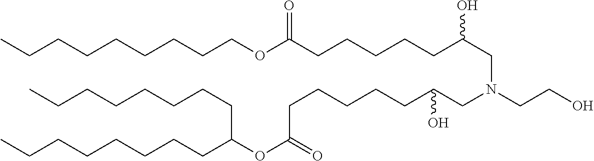 [SMILES: CCCCCCCCCOC(=O)CCCCCC(O)CN(CCO)CC(O)CCCCCC(=O)OC(CCCCCCCC)CCCCCCCC] |
| 20 | 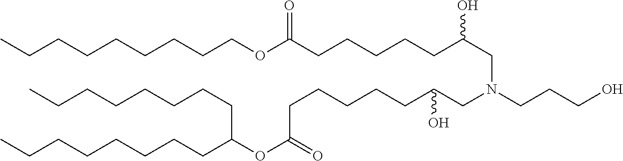 [SMILES: CCCCCCCCCOC(=O)CCCCCC(O)CN(CCCO)CC(O)CCCCCC(=O)OC(CCCCCCCC)CCCCCCCC] |
| 21 | 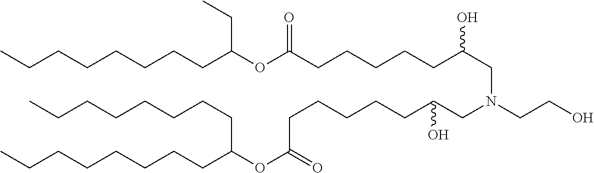 [SMILES: CCCCCCCCC(CC)OC(=O)CCCCCC(O)CN(CCO)CC(O)CCCCCC(=O)OC(CCCCCCCC)CCCCCCCC] |
| 22 | 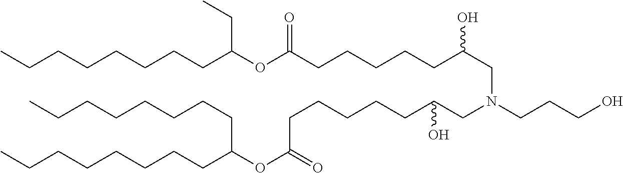 [SMILES: CCCCCCCCC(CC)OC(=O)CCCCCC(O)CN(CCCO)CC(O)CCCCCC(=O)OC(CCCCCCCC)CCCCCCCC] |
| 23 | 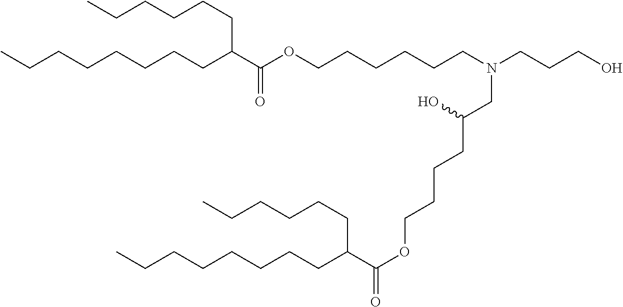 [SMILES: CCCCCCCCC(CCCCCC)C(=O)OCCCCCCN(CCCO)CC(O)CCCCOC(=O)C(CCCCCC)CCCCCCCC] |
| 24 | 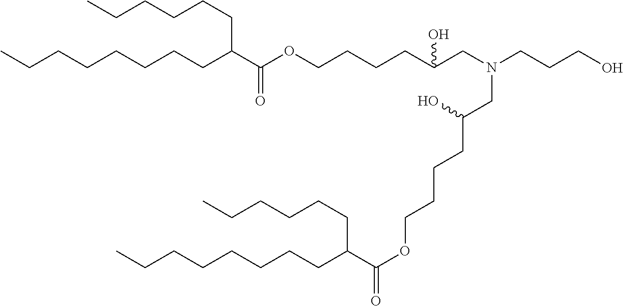 [SMILES: CCCCCCCCC(CCCCCC)C(=O)OCCCCC(O)CN(CCCO)CC(O)CCCCOC(=O)C(CCCCCC)CCCCCCCC] |
| 25 | 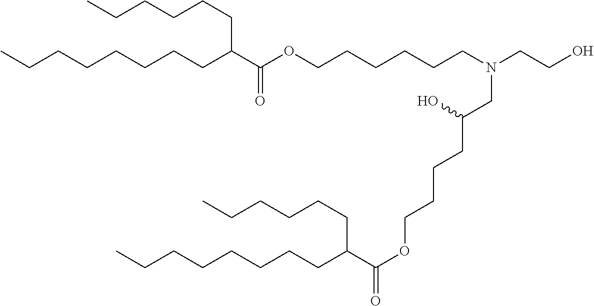 [SMILES: CCCCCCCCC(CCCCCC)C(=O)OCCCCCCN(CCO)CC(O)CCCCOC(=O)C(CCCCCC)CCCCCCCC] |
| 26 | 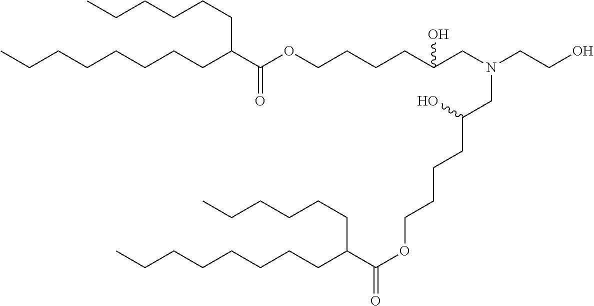 [SMILES: CCCCCCCCC(CCCCCC)C(=O)OCCCCC(O)CN(CCO)CC(O)CCCCOC(=O)C(CCCCCC)CCCCCCCC] |
| 27 | 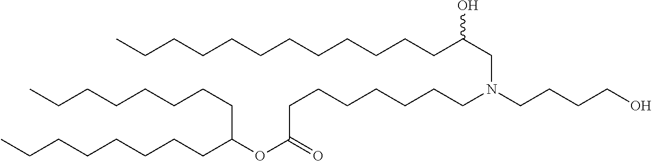 [SMILES: CCCCCCCCCCCCC(O)CN(CCCCO)CCCCCCCC(=O)OC(CCCCCCCC)CCCCCCCC] |
| 28 | 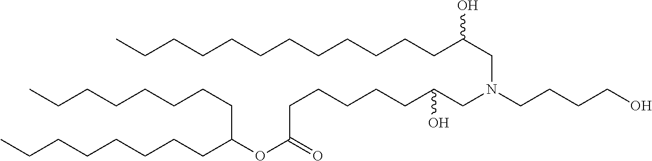 [SMILES: CCCCCCCCCCCCC(O)CN(CCCCO)CC(O)CCCCCC(=O)OC(CCCCCCCC)CCCCCCCC] |
| 29 | 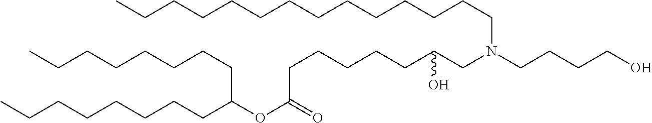 [SMILES: CCCCCCCCCCCCCCN(CCCCO)CC(O)CCCCCC(=O)OC(CCCCCCCC)CCCCCCCC] |
| 30 | 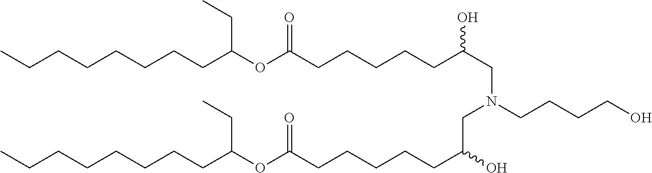 [SMILES: CCCCCCCCC(CC)OC(=O)CCCCCC(O)CN(CCCCO)CC(O)CCCCCC(=O)OC(CC)CCCCCCCC] |
| 31 | 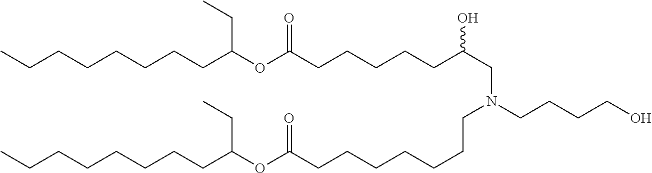 [SMILES: CCCCCCCCC(CC)OC(=O)CCCCCCCN(CCCCO)CC(O)CCCCCC(=O)OC(CC)CCCCCCCC] |
| 32 | 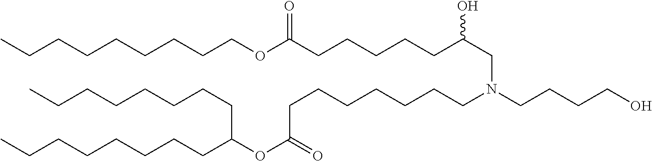 [SMILES: CCCCCCCCCOC(=O)CCCCCC(O)CN(CCCCO)CCCCCCCC(=O)OC(CCCCCCCC)CCCCCCCC] |
| 33 | 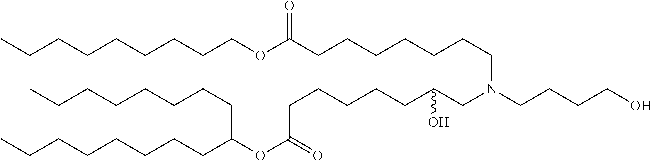 [SMILES: CCCCCCCCCOC(=O)CCCCCCCN(CCCCO)CC(O)CCCCCC(=O)OC(CCCCCCCC)CCCCCCCC] |
| 34 | 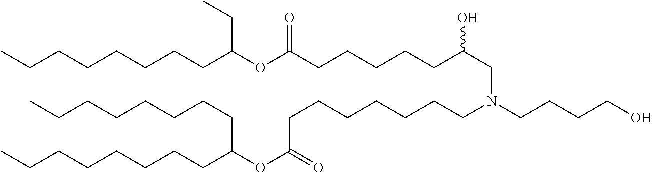 [SMILES: CCCCCCCCC(CC)OC(=O)CCCCCC(O)CN(CCCCO)CCCCCCCC(=O)OC(CCCCCCCC)CCCCCCCC] |
| 35 | 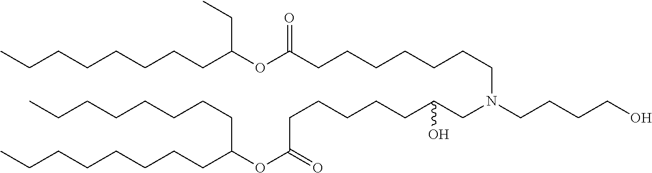 [SMILES: CCCCCCCCC(CC)OC(=O)CCCCCCCN(CCCCO)CC(O)CCCCCC(=O)OC(CCCCCCCC)CCCCCCCC] |
| 36 | 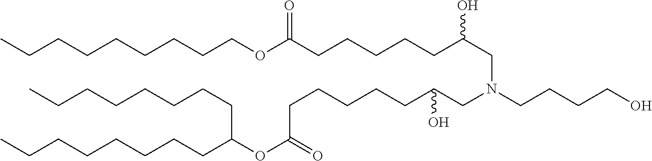 [SMILES: CCCCCCCCCOC(=O)CCCCCC(O)CN(CCCCO)CC(O)CCCCCC(=O)OC(CCCCCCCC)CCCCCCCC] |
| 37 | 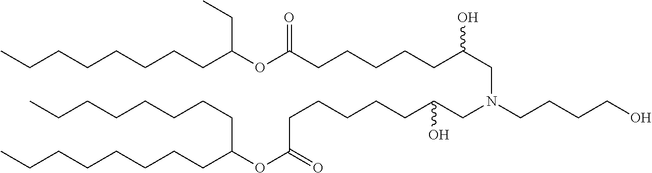 [SMILES: CCCCCCCCC(CC)OC(=O)CCCCCC(O)CN(CCCCO)CC(O)CCCCCC(=O)OC(CCCCCCCC)CCCCCCCC] |
| 38 | 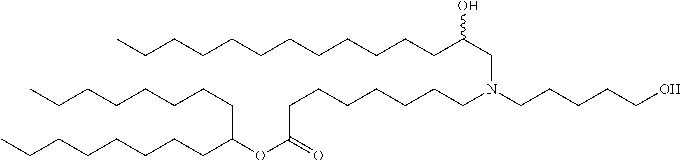 [SMILES: CCCCCCCCCCCCC(O)CN(CCCCCO)CCCCCCCC(=O)OC(CCCCCCCC)CCCCCCCC] |
| 39 | 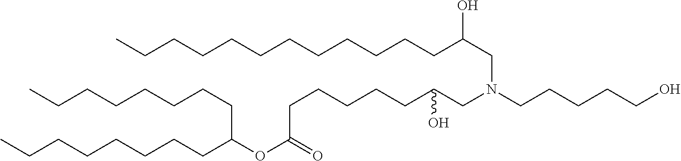 [SMILES: CCCCCCCCCCCCC(O)CN(CCCCCO)CC(O)CCCCCC(=O)OC(CCCCCCCC)CCCCCCCC] |
| 40 | 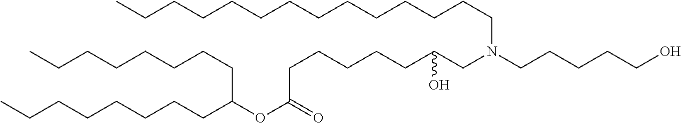 [SMILES: CCCCCCCCCCCCCCN(CCCCCO)CC(O)CCCCCC(=O)OC(CCCCCCCC)CCCCCCCC] |
| 41 | 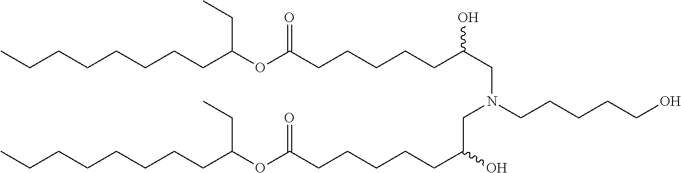 [SMILES: CCCCCCCCC(CC)OC(=O)CCCCCC(O)CN(CCCCCO)CC(O)CCCCCC(=O)OC(CC)CCCCCCCC] |
| 42 | 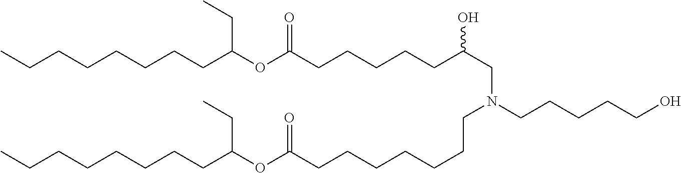 [SMILES: CCCCCCCCC(CC)OC(=O)CCCCCCCN(CCCCCO)CC(O)CCCCCC(=O)OC(CC)CCCCCCCC] |
| 43 | 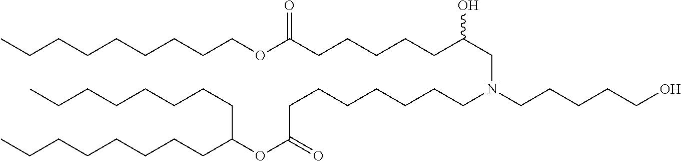 [SMILES: CCCCCCCCCOC(=O)CCCCCC(O)CN(CCCCCO)CCCCCCCC(=O)OC(CCCCCCCC)CCCCCCCC] |
| 44 | 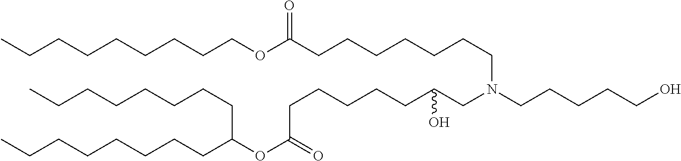 [SMILES: CCCCCCCCCOC(=O)CCCCCCCN(CCCCCO)CC(O)CCCCCC(=O)OC(CCCCCCCC)CCCCCCCC] |
| 45 | 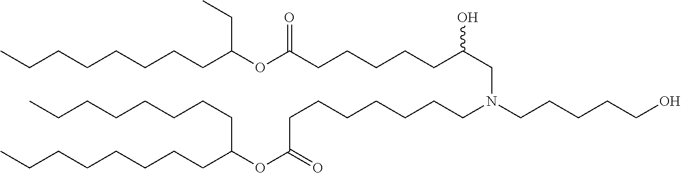 [SMILES: CCCCCCCCC(CC)OC(=O)CCCCCC(O)CN(CCCCCO)CCCCCCCC(=O)OC(CCCCCCCC)CCCCCCCC] |
| 46 | 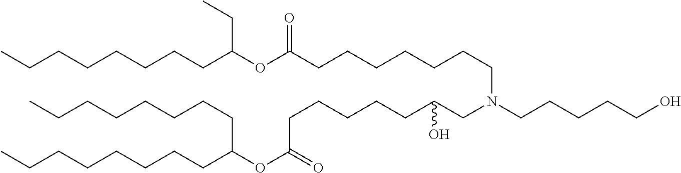 [SMILES: CCCCCCCCC(CC)OC(=O)CCCCCCCN(CCCCCO)CC(O)CCCCCC(=O)OC(CCCCCCCC)CCCCCCCC] |
| 47 | 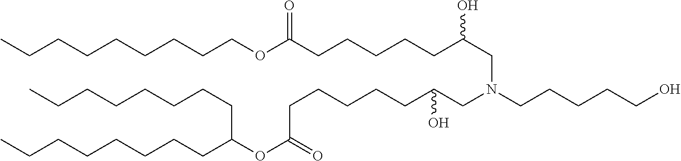 [SMILES: CCCCCCCCCOC(=O)CCCCCC(O)CN(CCCCCO)CC(O)CCCCCC(=O)OC(CCCCCCCC)CCCCCCCC] |
| 48 | 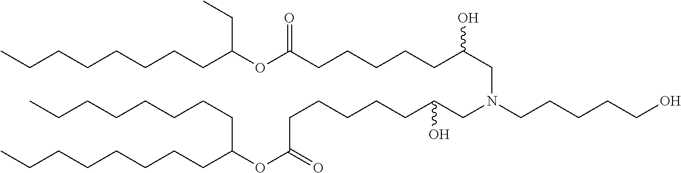 [SMILES: CCCCCCCCC(CC)OC(=O)CCCCCC(O)CN(CCCCCO)CC(O)CCCCCC(=O)OC(CCCCCCCC)CCCCCCCC] |
| 49 | 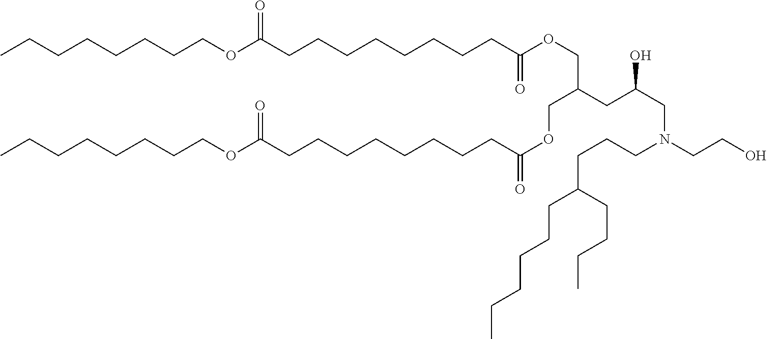 [SMILES: CCCCCCCCOC(=O)CCCCCCCCC(=O)OCC(COC(=O)CCCCCCCCC(=O)OCCCCCCCC)C[C@@H](O)CN(CCO)CCCC(CCCC)CCCCCC] |
| 50 | 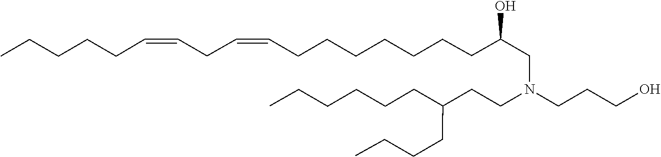 [SMILES: CCCCC/C=C\C/C=C\CCCCCCC[C@@H](O)CN(CCCO)CCC(CCCC)CCCCCC] |
| 51 | 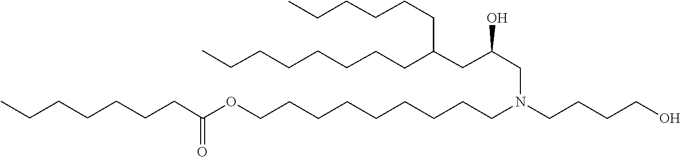 [SMILES: CCCCCCCCC(CCCCCC)C[C@@H](O)CN(CCCCO)CCCCCCCCCOC(=O)CCCCCCC] |
| 52 | 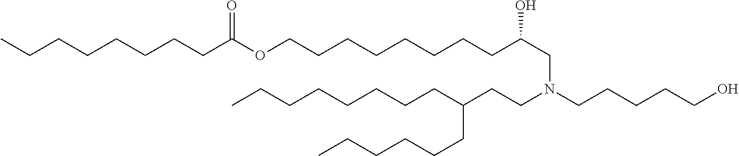 [SMILES: CCCCCCCCC(=O)OCCCCCCCC[C@H](O)CN(CCCCCO)CCC(CCCCCC)CCCCCCCC] |
| 53 | 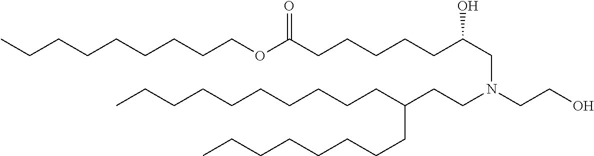 [SMILES: CCCCCCCCCCC(CCCCCCCC)CCN(CCO)C[C@@H](O)CCCCCC(=O)OCCCCCCCCC] |
| 54 | 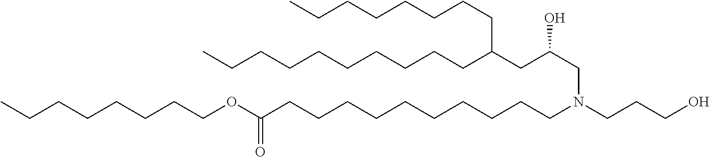 [SMILES: CCCCCCCCCCC(CCCCCCCC)C[C@H](O)CN(CCCO)CCCCCCCCCCC(=O)OCCCCCCCC] |
| 55 | 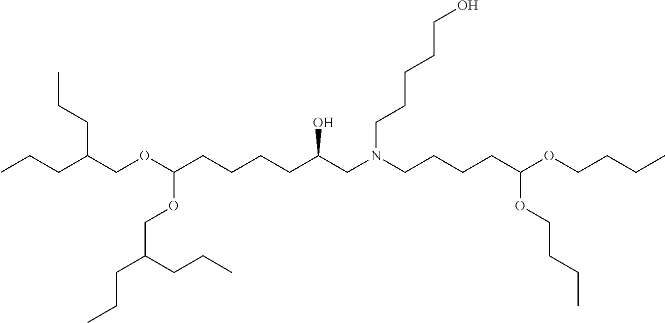 [SMILES: CCCCOC(CCCCN(CCCCCO)C[C@H](O)CCCCC(OCC(CCC)CCC)OCC(CCC)CCC)OCCCC] |
| 56 | 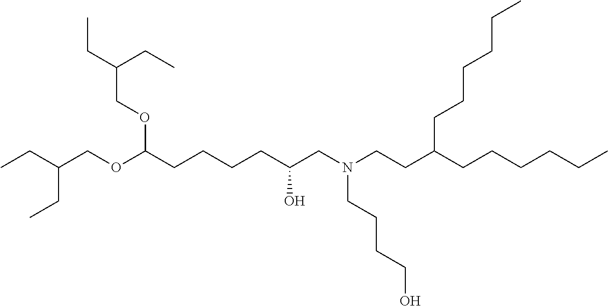 [SMILES: CCCCCCC(CCCCCC)CCN(CCCCO)C[C@H](O)CCCCC(OCC(CC)CC)OCC(CC)CC] |
| 57 | 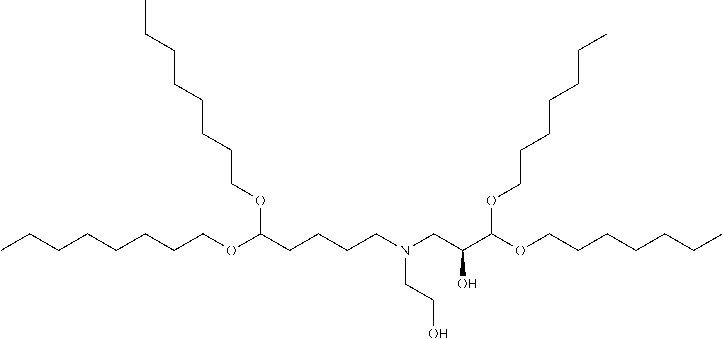 [SMILES: CCCCCCCCOC(CCCCN(CCO)C[C@H](O)C(OCCCCCCC)OCCCCCCC)OCCCCCCCC] |
| 58 | 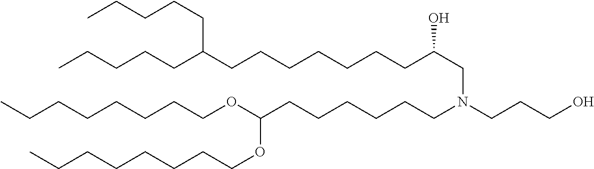 [SMILES: CCCCCCCCOC(CCCCCCN(CCCO)C[C@@H](O)CCCCCCCC(CCCCC)CCCCC)OCCCCCCCC] |
| 59 | 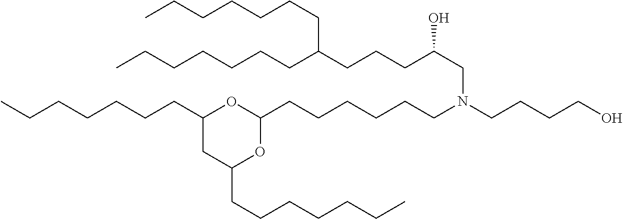 [SMILES: CCCCCCCC(CCCCCCC)CCC[C@H](O)CN(CCCCO)CCCCCCC1OC(CCCCCCC)CC(CCCCCCC)O1] |
| 60 | 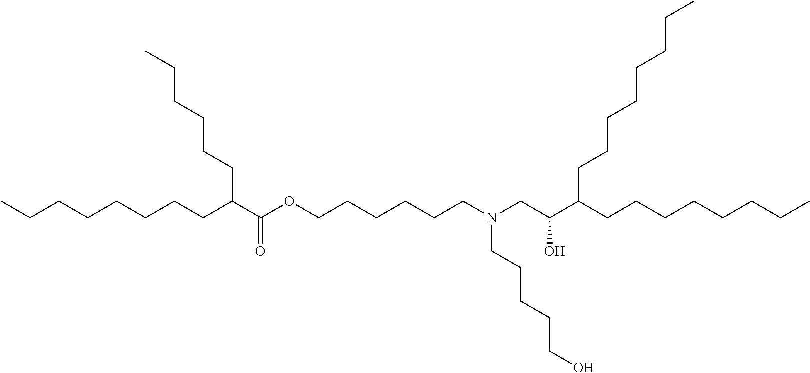 [SMILES: CCCCCCCCC(CCCCCC)C(=O)OCCCCCCN(CCCCCO)C[C@@H](O)C(CCCCCCCC)CCCCCCCC] |
| 61 | 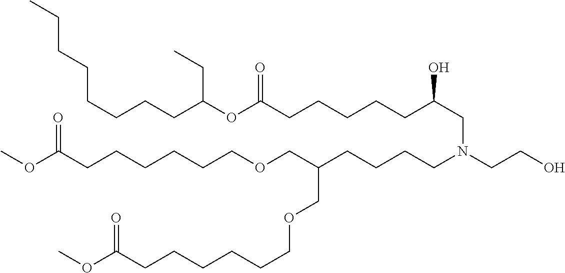 [SMILES: CCCCCCCCC(CC)OC(=O)CCCCC[C@@H](O)CN(CCO)CCCCC(COCCCCCCC(=O)OC)COCCCCCCC(=O)OC] |
| 62 | 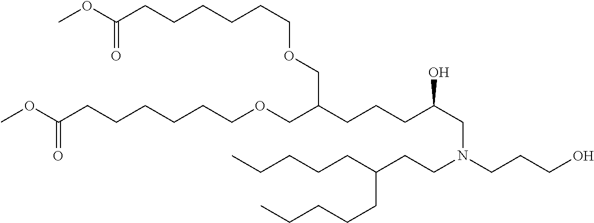 [SMILES: CCCCCC(CCCCC)CCN(CCCO)C[C@H](O)CCCC(COCCCCCCC(=O)OC)COCCCCCCC(=O)OC] |
| 63 | 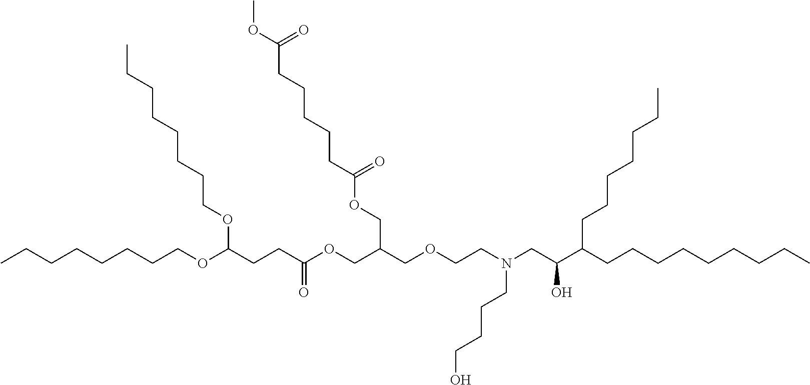 [SMILES: CCCCCCCCCC(CCCCCCC)[C@@H](O)CN(CCCCO)CCOCC(COC(=O)CCCCCC(=O)OC)COC(=O)CCC(OCCCCCCCC)OCCCCCCCC] |
| 64 | 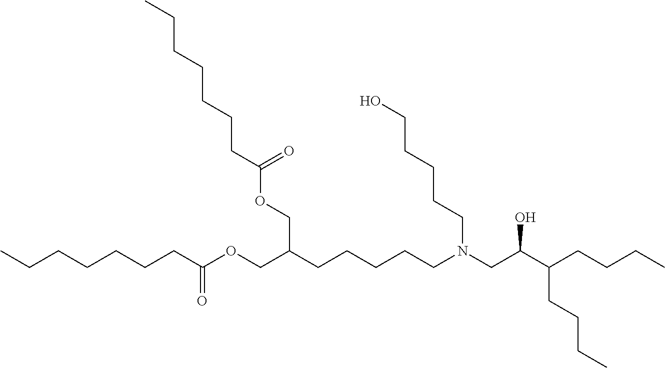 [SMILES: CCCCCCCC(=O)OCC(CCCCCN(CCCCCO)C[C@@H](O)C(CCCC)CCCC)COC(=O)CCCCCCC] |
| 65 | 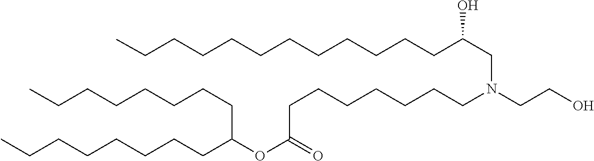 [SMILES: CCCCCCCCCCCC[C@H](O)CN(CCO)CCCCCCCC(=O)OC(CCCCCCCC)CCCCCCCC] |
| 66 | 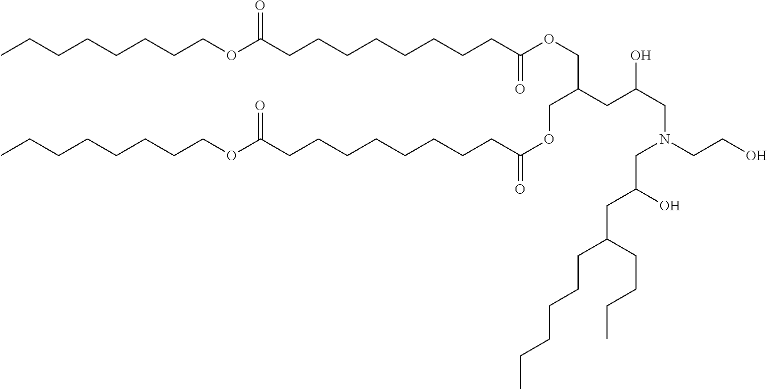 [SMILES: CCCCCCCCOC(=O)CCCCCCCCC(=O)OCC(COC(=O)CCCCCCCCC(=O)OCCCCCCCC)CC(O)CN(CCO)CC(O)CC(CCCC)CCCCCC] |
| 67 | 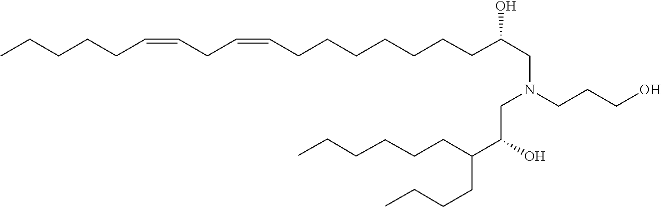 [SMILES: CCCCC/C=C\C/C=C\CCCCCCC[C@H](O)CN(CCCO)C[C@H](O)C(CCCC)CCCCCC] |
| 68 | 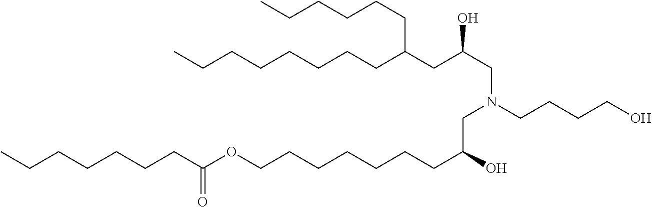 [SMILES: CCCCCCCCC(CCCCCC)C[C@@H](O)CN(CCCCO)C[C@@H](O)CCCCCCCOC(=O)CCCCCCC] |
| 69 | 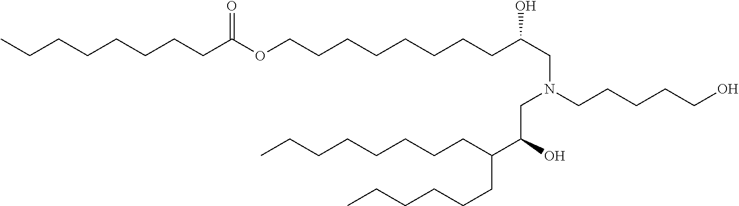 [SMILES: CCCCCCCCC(=O)OCCCCCCCC[C@H](O)CN(CCCCCO)C[C@@H](O)C(CCCCCC)CCCCCCCC] |
| 70 | 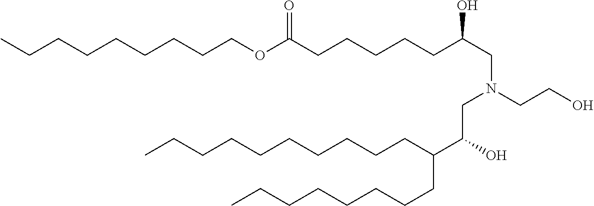 [SMILES: CCCCCCCCCCC(CCCCCCCC)[C@@H](O)CN(CCO)C[C@H](O)CCCCCC(=O)OCCCCCCCCC] |
| 71 | 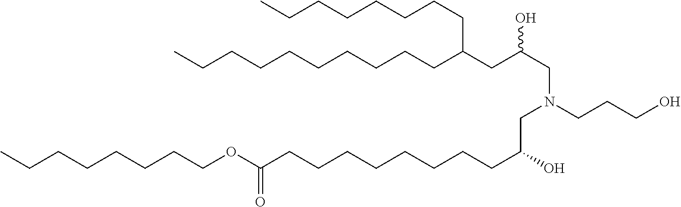 [SMILES: CCCCCCCCCCC(CCCCCCCC)CC(O)CN(CCCO)C[C@H](O)CCCCCCCCC(=O)OCCCCCCCC] |
| 72 | 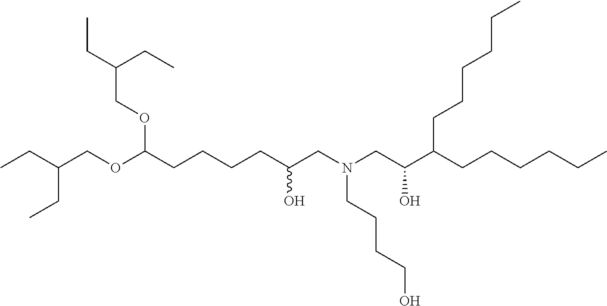 [SMILES: CCCCCCC(CCCCCC)[C@H](O)CN(CCCCO)CC(O)CCCCC(OCC(CC)CC)OCC(CC)CC] |
| 73 | 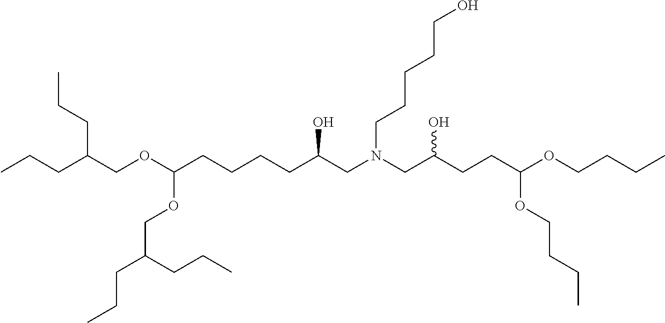 [SMILES: CCCCOC(CCC(O)CN(CCCCCO)C[C@H](O)CCCCC(OCC(CCC)CCC)OCC(CCC)CCC)OCCCC] |
| 74 | 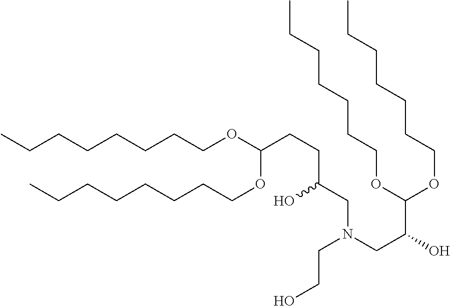 [SMILES: CCCCCCCCOC(CCC(O)CN(CCO)C[C@@H](O)C(OCCCCCCC)OCCCCCCC)OCCCCCCCC] |
| 75 | 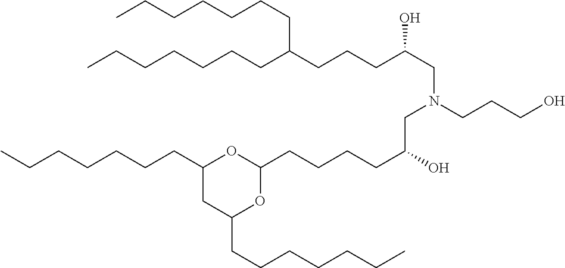 [SMILES: CCCCCCCC(CCCCCCC)CCC[C@H](O)CN(CCCO)C[C@H](O)CCCCC1OC(CCCCCCC)CC(CCCCCCC)O1] |
| 76 | 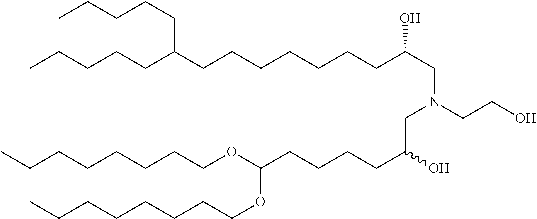 [SMILES: CCCCCCCCOC(CCCCC(O)CN(CCO)C[C@@H](O)CCCCCCCC(CCCCC)CCCCC)OCCCCCCCC] |
| 77 | 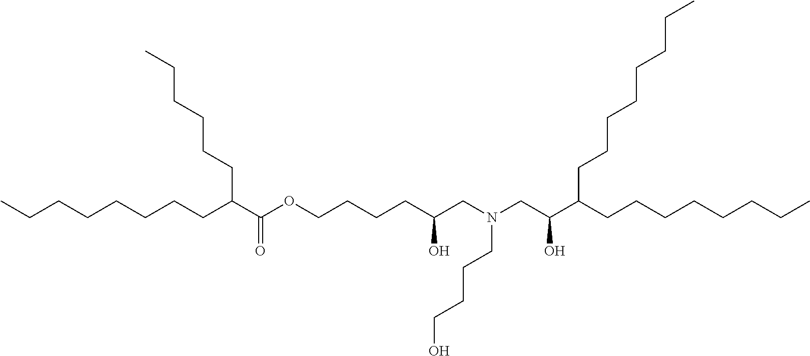 [SMILES: CCCCCCCCC(CCCCCC)C(=O)OCCCC[C@H](O)CN(CCCCO)C[C@H](O)C(CCCCCCCC)CCCCCCCC] |
| 78 | 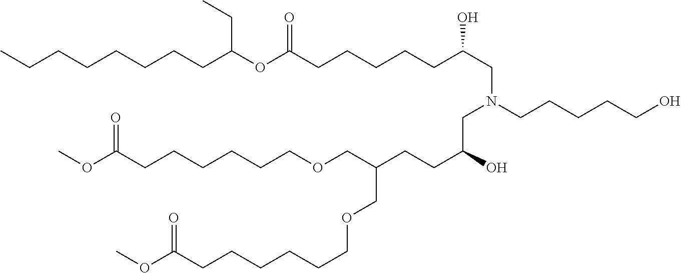 [SMILES: CCCCCCCCC(CC)OC(=O)CCCCC[C@H](O)CN(CCCCCO)C[C@@H](O)CCC(COCCCCCCC(=O)OC)COCCCCCCC(=O)OC] |
| 79 | 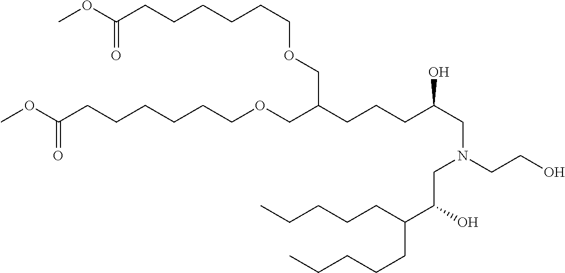 [SMILES: CCCCCC(CCCCC)[C@@H](O)CN(CCO)C[C@H](O)CCCC(COCCCCCCC(=O)OC)COCCCCCCC(=O)OC] |
| 80 | 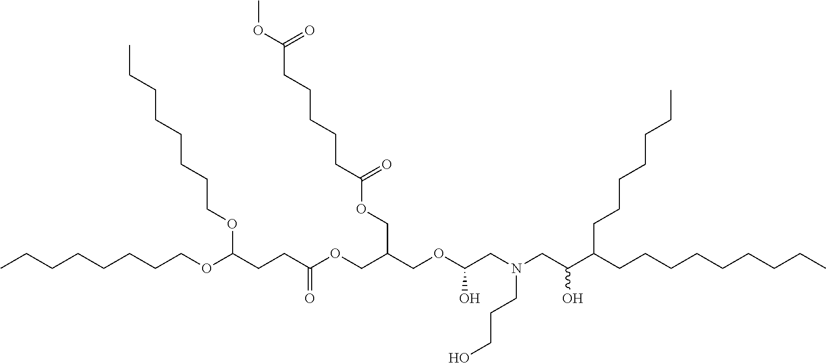 [SMILES: CCCCCCCCCC(CCCCCCC)C(O)CN(CCCO)C[C@H](O)OCC(COC(=O)CCCCCC(=O)OC)COC(=O)CCC(OCCCCCCCC)OCCCCCCCC] |
| 81 | 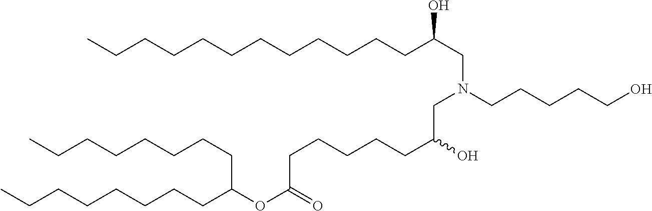 [SMILES: CCCCCCCCCCCC[C@@H](O)CN(CCCCCO)CC(O)CCCCCC(=O)OC(CCCCCCCC)CCCCCCCC] |
| 82 | 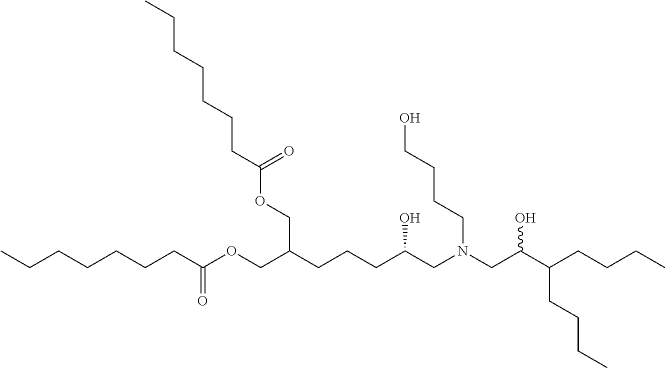 [SMILES: CCCCCCCC(=O)OCC(CCC[C@H](O)CN(CCCCO)CC(O)C(CCCC)CCCC)COC(=O)CCCCCCC] |
| 83 | 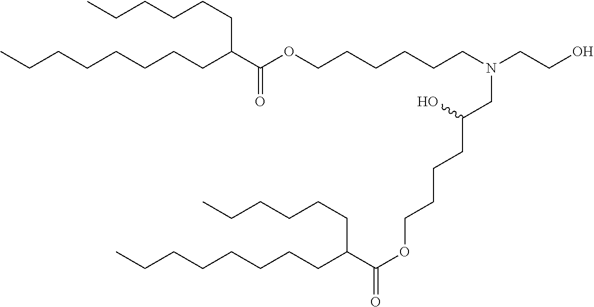 [SMILES: CCCCCCCCC(CCCCCC)C(=O)OCCCCCCN(CCO)CC(O)CCCCOC(=O)C(CCCCCC)CCCCCCCC] |
| 84 | 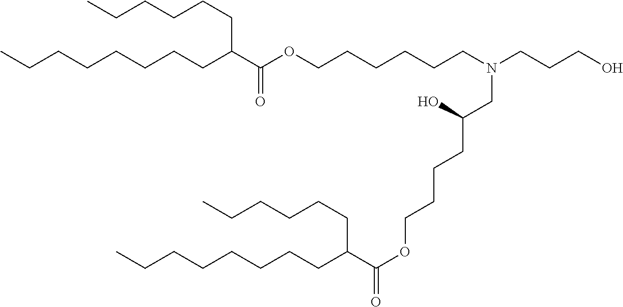 [SMILES: CCCCCCCCC(CCCCCC)C(=O)OCCCCCCN(CCCO)C[C@H](O)CCCCOC(=O)C(CCCCCC)CCCCCCCC] |
| 85 | 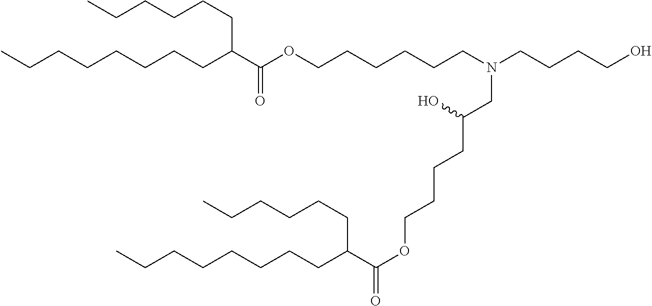 [SMILES: CCCCCCCCC(CCCCCC)C(=O)OCCCCCCN(CCCCO)CC(O)CCCCOC(=O)C(CCCCCC)CCCCCCCC] |
| 86 | 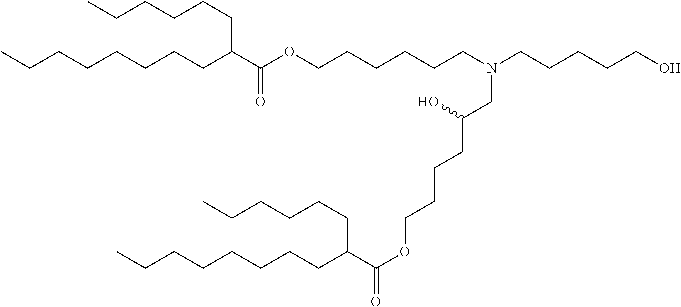 [SMILES: CCCCCCCCC(CCCCCC)C(=O)OCCCCCCN(CCCCCO)CC(O)CCCCOC(=O)C(CCCCCC)CCCCCCCC] |
| 87 | 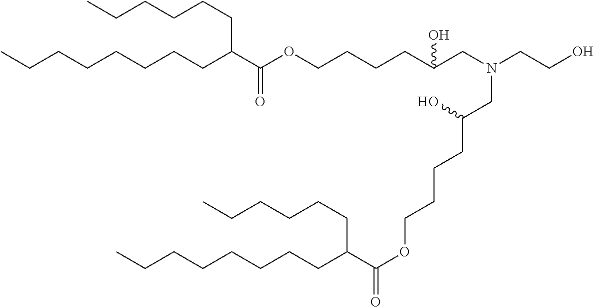 [SMILES: CCCCCCCCC(CCCCCC)C(=O)OCCCCC(O)CN(CCO)CC(O)CCCCOC(=O)C(CCCCCC)CCCCCCCC] |
| 88 | 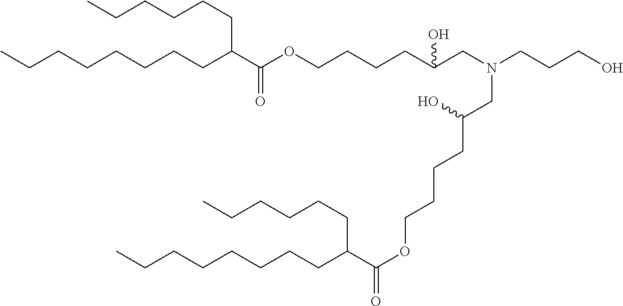 [SMILES: CCCCCCCCC(CCCCCC)C(=O)OCCCCC(O)CN(CCCO)CC(O)CCCCOC(=O)C(CCCCCC)CCCCCCCC] |
| 89 | 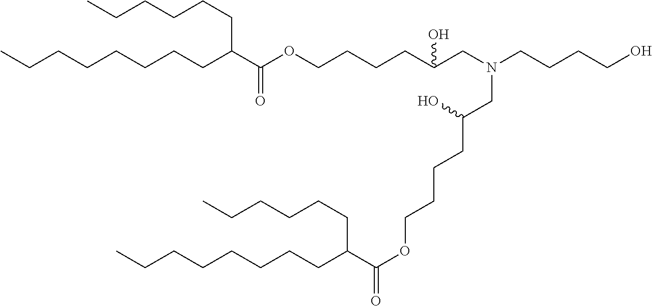 [SMILES: CCCCCCCCC(CCCCCC)C(=O)OCCCCC(O)CN(CCCCO)CC(O)CCCCOC(=O)C(CCCCCC)CCCCCCCC] |
| 90 | 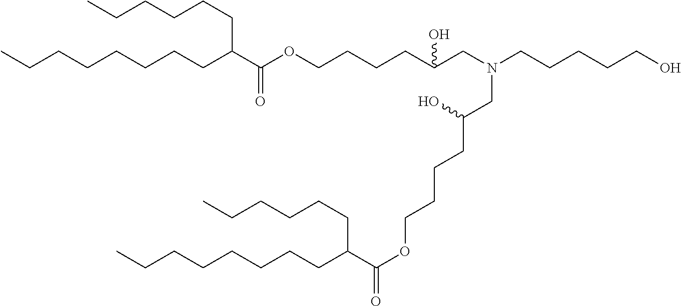 [SMILES: CCCCCCCCC(CCCCCC)C(=O)OCCCCC(O)CN(CCCCCO)CC(O)CCCCOC(=O)C(CCCCCC)CCCCCCCC] |
| 91 | 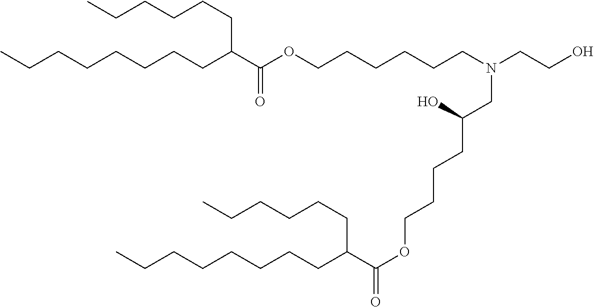 [SMILES: CCCCCCCCC(CCCCCC)C(=O)OCCCCCCN(CCO)C[C@H](O)CCCCOC(=O)C(CCCCCC)CCCCCCCC] |
| 92 | 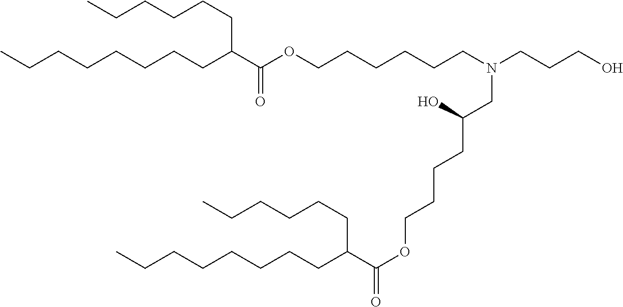 [SMILES: CCCCCCCCC(CCCCCC)C(=O)OCCCCCCN(CCCO)C[C@H](O)CCCCOC(=O)C(CCCCCC)CCCCCCCC] |
| 93 | 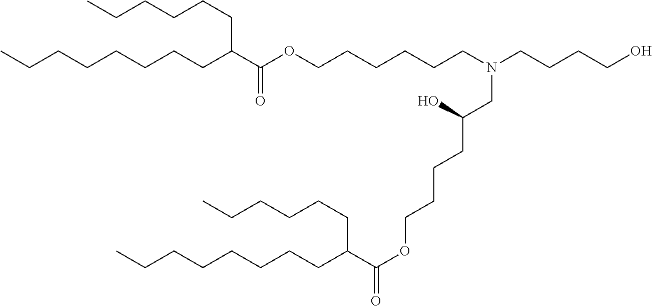 [SMILES: CCCCCCCCC(CCCCCC)C(=O)OCCCCCCN(CCCCO)C[C@H](O)CCCCOC(=O)C(CCCCCC)CCCCCCCC] |
| 94 | 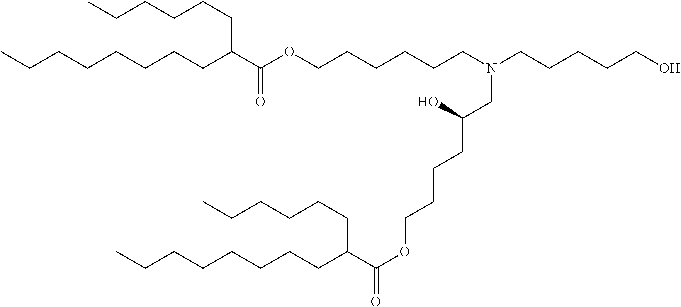 [SMILES: CCCCCCCCC(CCCCCC)C(=O)OCCCCCCN(CCCCCO)C[C@H](O)CCCCOC(=O)C(CCCCCC)CCCCCCCC] |
| 95 | 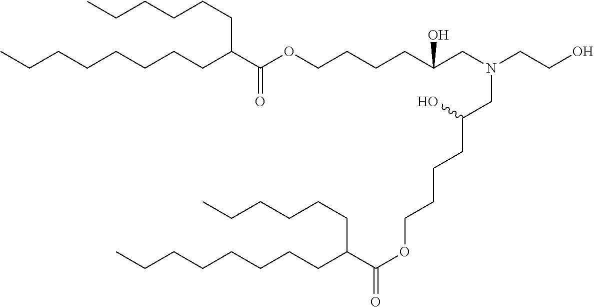 [SMILES: CCCCCCCCC(CCCCCC)C(=O)OCCCCC(O)CN(CCO)C[C@@H](O)CCCCOC(=O)C(CCCCCC)CCCCCCCC] |
| 96 | 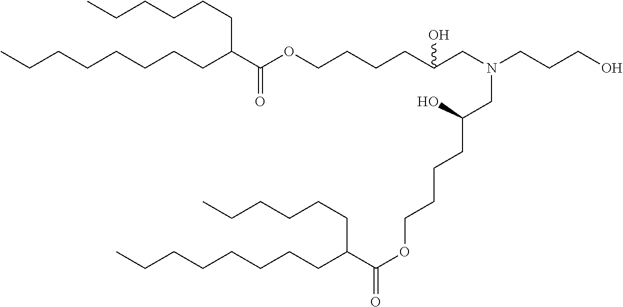 [SMILES: CCCCCCCCC(CCCCCC)C(=O)OCCCCC(O)CN(CCCO)C[C@H](O)CCCCOC(=O)C(CCCCCC)CCCCCCCC] |
| 97 | 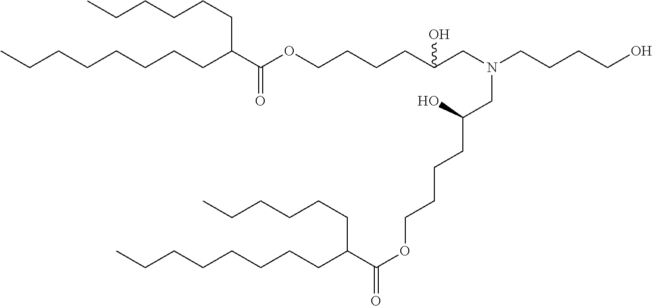 [SMILES: CCCCCCCCC(CCCCCC)C(=O)OCCCCC(O)CN(CCCCO)C[C@H](O)CCCCOC(=O)C(CCCCCC)CCCCCCCC] |
| 98 | 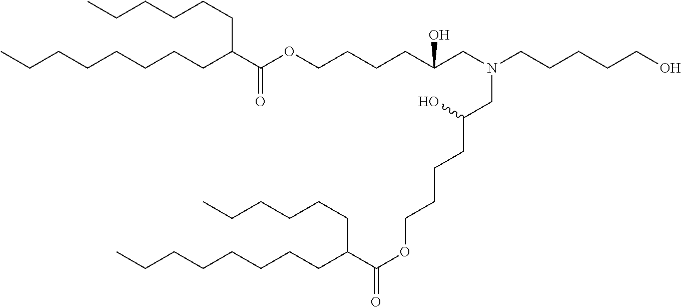 [SMILES: CCCCCCCCC(CCCCCC)C(=O)OCCCCC(O)CN(CCCCCO)C[C@@H](O)CCCCOC(=O)C(CCCCCC)CCCCCCCC] |
| 99 | 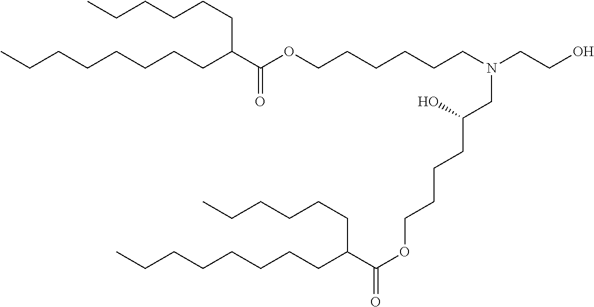 [SMILES: CCCCCCCCC(CCCCCC)C(=O)OCCCCCCN(CCO)C[C@@H](O)CCCCOC(=O)C(CCCCCC)CCCCCCCC] |
| 100 | 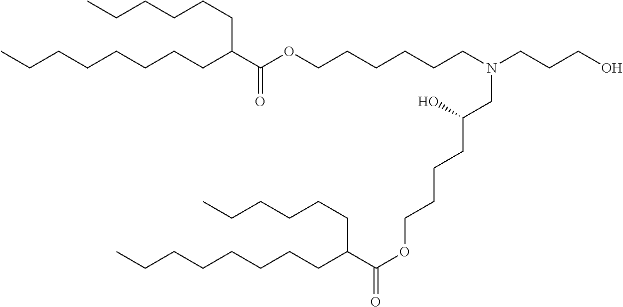 [SMILES: CCCCCCCCC(CCCCCC)C(=O)OCCCCCCN(CCCO)C[C@@H](O)CCCCOC(=O)C(CCCCCC)CCCCCCCC] |
| 101 | 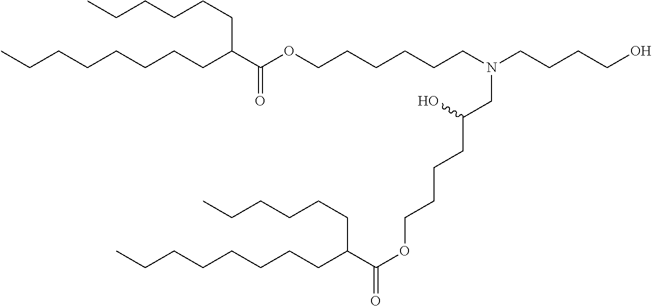 [SMILES: CCCCCCCCC(CCCCCC)C(=O)OCCCCCCN(CCCCO)CC(O)CCCCOC(=O)C(CCCCCC)CCCCCCCC] |
| 102 | 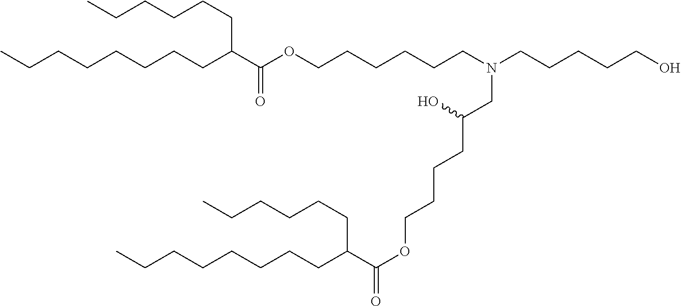 [SMILES: CCCCCCCCC(CCCCCC)C(=O)OCCCCCCN(CCCCCO)CC(O)CCCCOC(=O)C(CCCCCC)CCCCCCCC] |
| 103 | 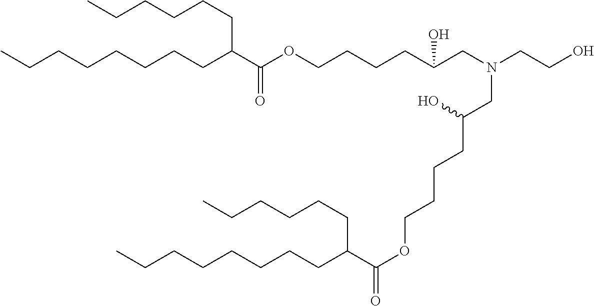 [SMILES: CCCCCCCCC(CCCCCC)C(=O)OCCCCC(O)CN(CCO)C[C@H](O)CCCCOC(=O)C(CCCCCC)CCCCCCCC] |
| 104 | 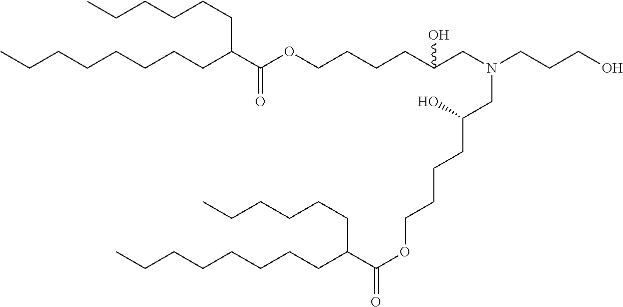 [SMILES: CCCCCCCCC(CCCCCC)C(=O)OCCCCC(O)CN(CCCO)C[C@@H](O)CCCCOC(=O)C(CCCCCC)CCCCCCCC] |
| 105 | 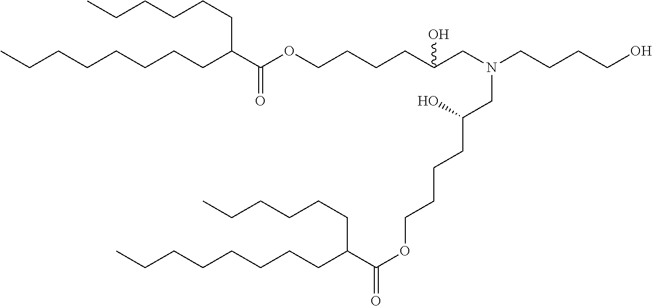 [SMILES: CCCCCCCCC(CCCCCC)C(=O)OCCCCC(O)CN(CCCCO)C[C@@H](O)CCCCOC(=O)C(CCCCCC)CCCCCCCC] |
| 106 | 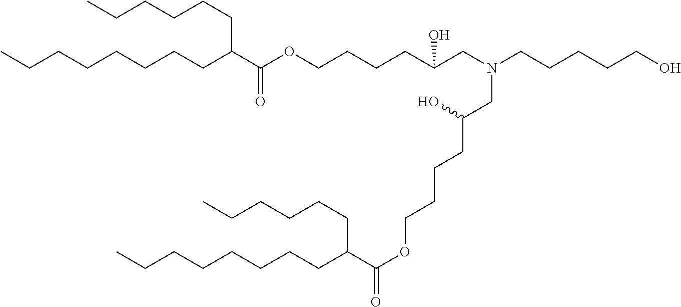 [SMILES: CCCCCCCCC(CCCCCC)C(=O)OCCCCC(O)CN(CCCCCO)C[C@H](O)CCCCOC(=O)C(CCCCCC)CCCCCCCC] |
| 107 | 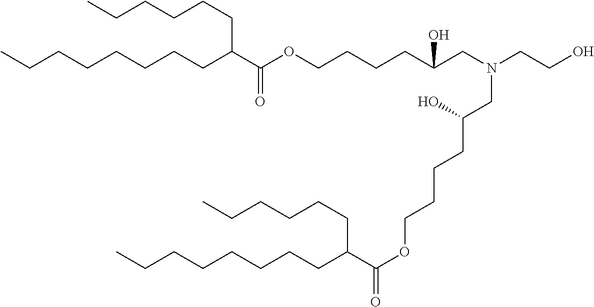 [SMILES: CCCCCCCCC(CCCCCC)C(=O)OCCCC[C@H](O)CN(CCO)C[C@@H](O)CCCCOC(=O)C(CCCCCC)CCCCCCCC] |
| 108 | 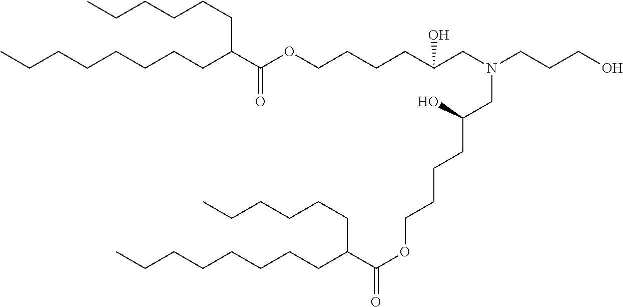 [SMILES: CCCCCCCCC(CCCCCC)C(=O)OCCCC[C@@H](O)CN(CCCO)C[C@H](O)CCCCOC(=O)C(CCCCCC)CCCCCCCC] |
| 109 | 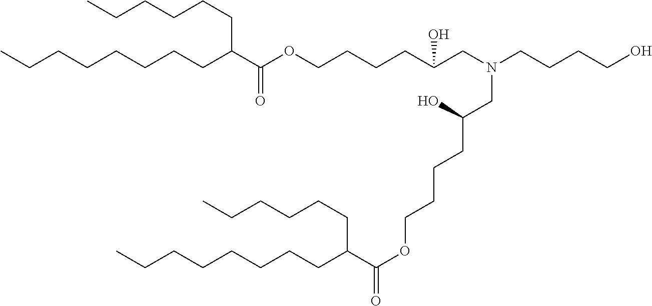 [SMILES: CCCCCCCCC(CCCCCC)C(=O)OCCCC[C@@H](O)CN(CCCCO)C[C@H](O)CCCCOC(=O)C(CCCCCC)CCCCCCCC] |
| 110 | 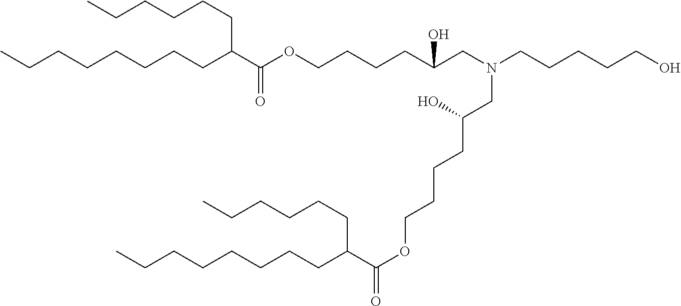 [SMILES: CCCCCCCCC(CCCCCC)C(=O)OCCCC[C@H](O)CN(CCCCCO)C[C@@H](O)CCCCOC(=O)C(CCCCCC)CCCCCCCC] |
| 111 | 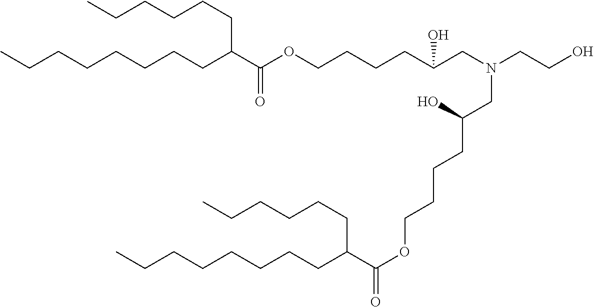 [SMILES: CCCCCCCCC(CCCCCC)C(=O)OCCCC[C@@H](O)CN(CCO)C[C@H](O)CCCCOC(=O)C(CCCCCC)CCCCCCCC] |
| 112 | 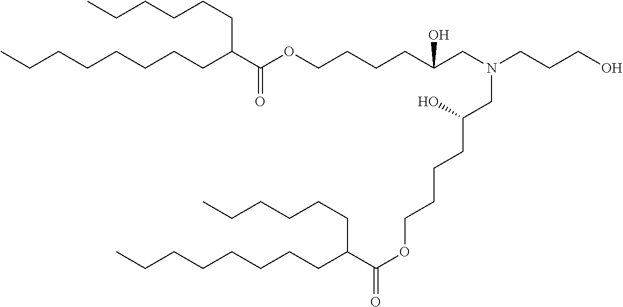 [SMILES: CCCCCCCCC(CCCCCC)C(=O)OCCCC[C@H](O)CN(CCCO)C[C@@H](O)CCCCOC(=O)C(CCCCCC)CCCCCCCC] |
| 113 | 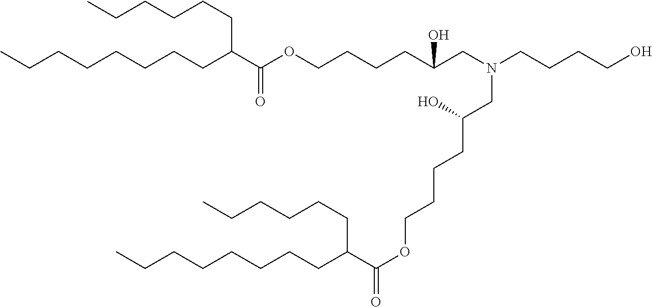 [SMILES: CCCCCCCCC(CCCCCC)C(=O)OCCCC[C@H](O)CN(CCCCO)C[C@@H](O)CCCCOC(=O)C(CCCCCC)CCCCCCCC] |
| 114 | 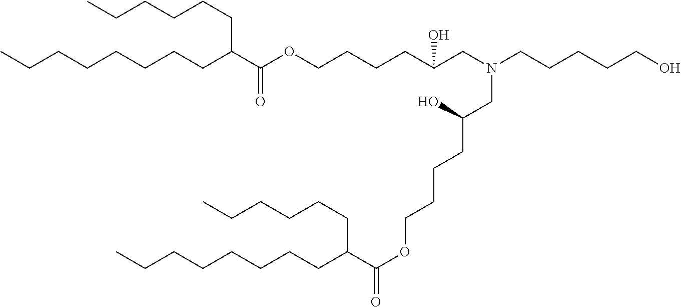 [SMILES: CCCCCCCCC(CCCCCC)C(=O)OCCCC[C@@H](O)CN(CCCCCO)C[C@H](O)CCCCOC(=O)C(CCCCCC)CCCCCCCC] |
| 115 | 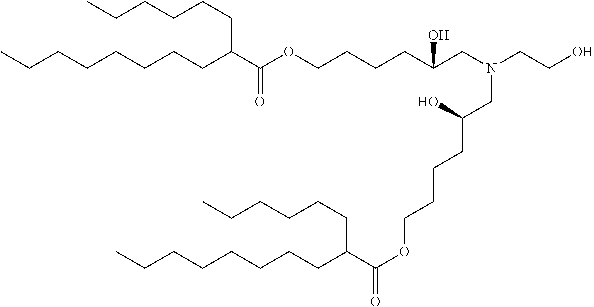 [SMILES: CCCCCCCCC(CCCCCC)C(=O)OCCCC[C@@H](O)CN(CCO)C[C@@H](O)CCCCOC(=O)C(CCCCCC)CCCCCCCC] |
| 116 | 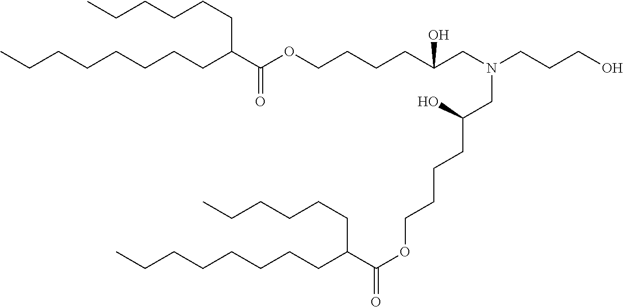 [SMILES: CCCCCCCCC(CCCCCC)C(=O)OCCCC[C@@H](O)CN(CCCO)C[C@@H](O)CCCCOC(=O)C(CCCCCC)CCCCCCCC] |
| 117 | 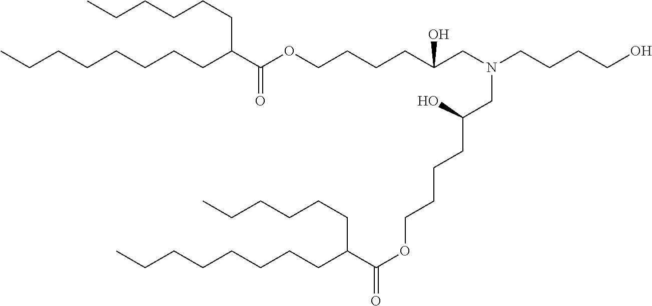 [SMILES: CCCCCCCCC(CCCCCC)C(=O)OCCCC[C@@H](O)CN(CCCCO)C[C@@H](O)CCCCOC(=O)C(CCCCCC)CCCCCCCC] |
| 118 | 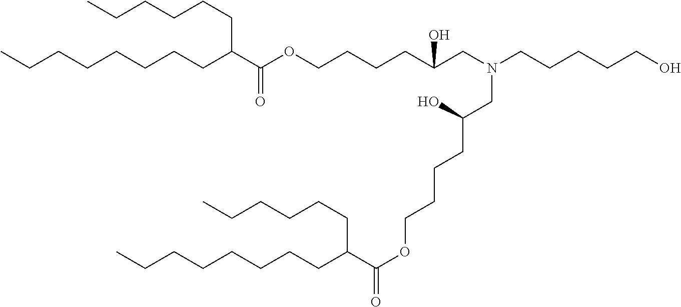 [SMILES: CCCCCCCCC(CCCCCC)C(=O)OCCCC[C@@H](O)CN(CCCCCO)C[C@@H](O)CCCCOC(=O)C(CCCCCC)CCCCCCCC] |
| 119 | 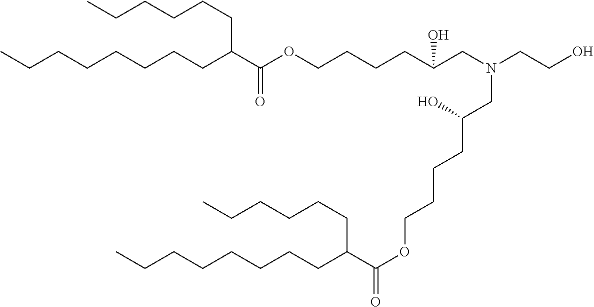 [SMILES: CCCCCCCCC(CCCCCC)C(=O)OCCCC[C@@H](O)CN(CCO)C[C@@H](O)CCCCOC(=O)C(CCCCCC)CCCCCCCC] |
| 120 | 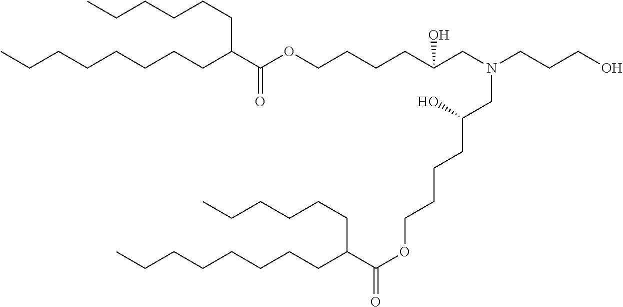 [SMILES: CCCCCCCCC(CCCCCC)C(=O)OCCCC[C@@H](O)CN(CCCO)C[C@@H](O)CCCCOC(=O)C(CCCCCC)CCCCCCCC] |
| 121 | 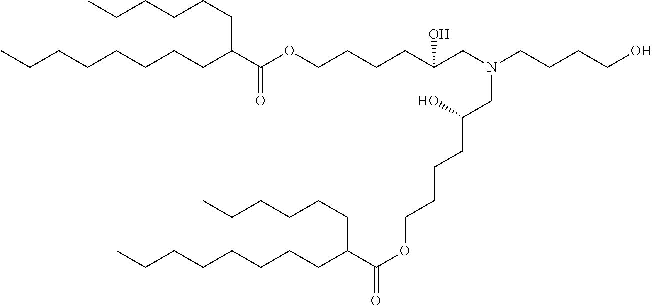 [SMILES: CCCCCCCCC(CCCCCC)C(=O)OCCCC[C@@H](O)CN(CCCCO)C[C@@H](O)CCCCOC(=O)C(CCCCCC)CCCCCCCC] |
| 122 | 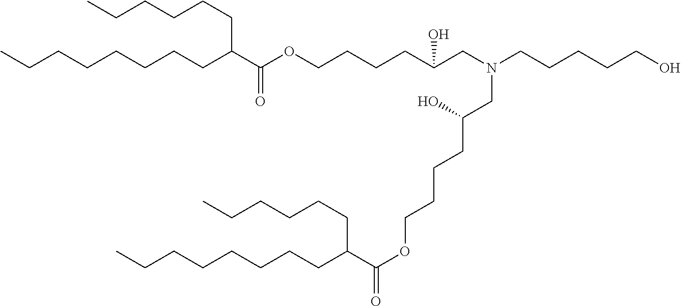 [SMILES: CCCCCCCCC(CCCCCC)C(=O)OCCCC[C@@H](O)CN(CCCCCO)C[C@@H](O)CCCCOC(=O)C(CCCCCC)CCCCCCCC] |
| 123 | 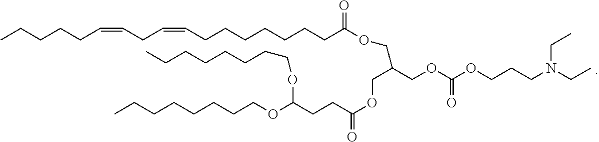 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| 124 | 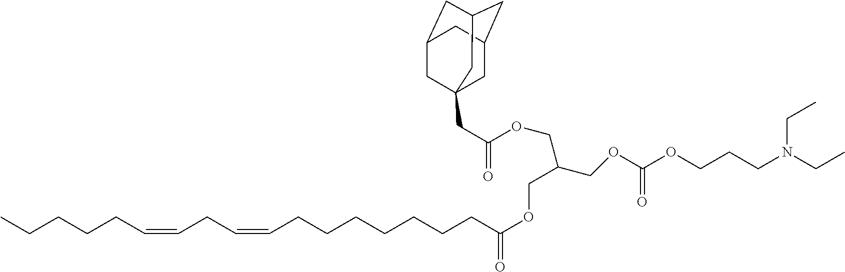 |
| 125 | 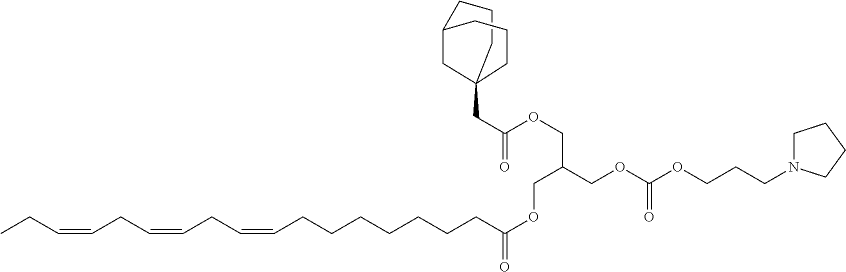 |
| 126 | 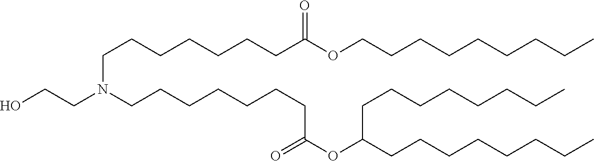 [SMILES: CCCCCCCCCOC(=O)CCCCCCCN(CCO)CCCCCCCC(=O)OC(CCCCCCCC)CCCCCCCC] |
| 127 | 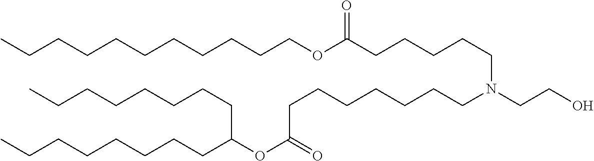 [SMILES: CCCCCCCCCCCOC(=O)CCCCCN(CCO)CCCCCCCC(=O)OC(CCCCCCCC)CCCCCCCC] |
| 128 | 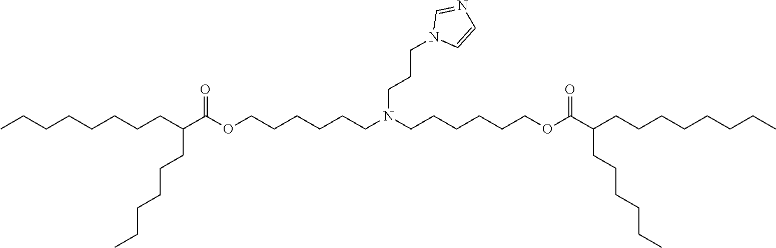 [SMILES: CCCCCCCCC(CCCCCC)C(=O)OCCCCCCN(CCCCCCOC(=O)C(CCCCCC)CCCCCCCC)CCCn1ccnc1] |
| 129 | 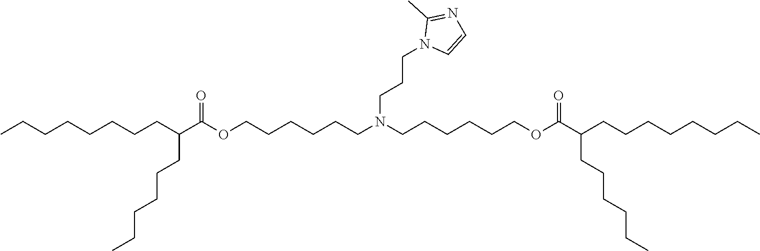 [SMILES: CCCCCCCCC(CCCCCC)C(=O)OCCCCCCN(CCCCCCOC(=O)C(CCCCCC)CCCCCCCC)CCCn1ccnc1C] |
| 130 | 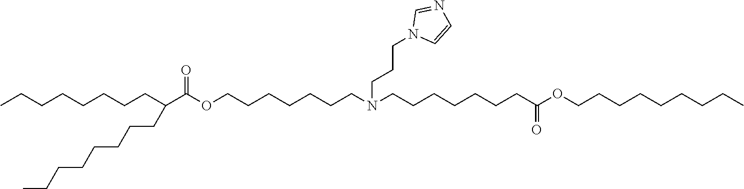 [SMILES: CCCCCCCCCOC(=O)CCCCCCCN(CCCCCCCOC(=O)C(CCCCCCCC)CCCCCCCC)CCCn1ccnc1] |
| 131 | 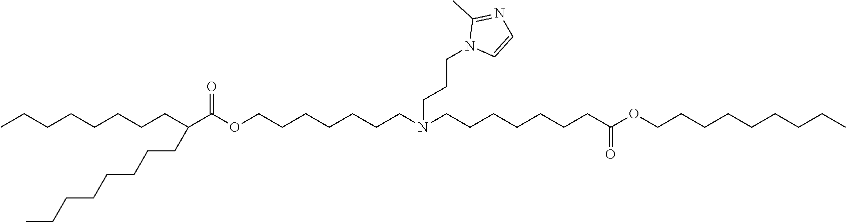 [SMILES: CCCCCCCCCOC(=O)CCCCCCCN(CCCCCCCOC(=O)C(CCCCCCCC)CCCCCCCC)CCCn1ccnc1C] |
| 132 | 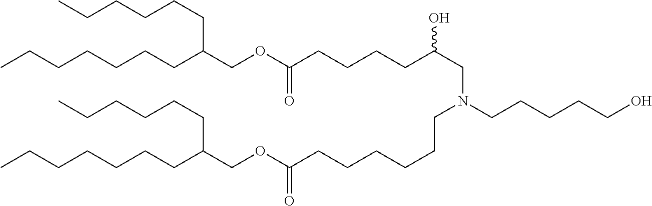 [SMILES: CCCCCCCC(CCCCCC)COC(=O)CCCCCCN(CCCCCO)CC(O)CCCCC(=O)OCC(CCCCCC)CCCCCCC] |
| 133 | 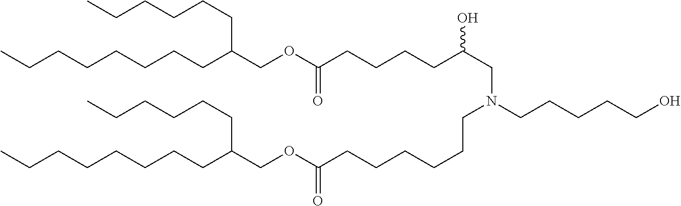 [SMILES: CCCCCCCCC(CCCCCC)COC(=O)CCCCCCN(CCCCCO)CC(O)CCCCC(=O)OCC(CCCCCC)CCCCCCCC] |
| 134 | 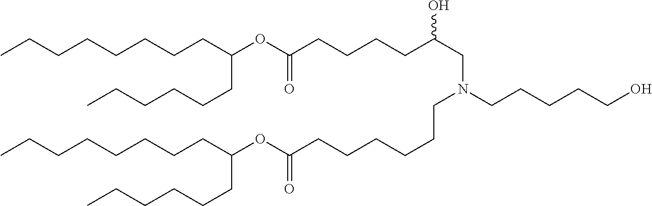 [SMILES: CCCCCCCCC(CCCCCC)OC(=O)CCCCCCN(CCCCCO)CC(O)CCCCC(=O)OC(CCCCCC)CCCCCCCC] |
| 135 | 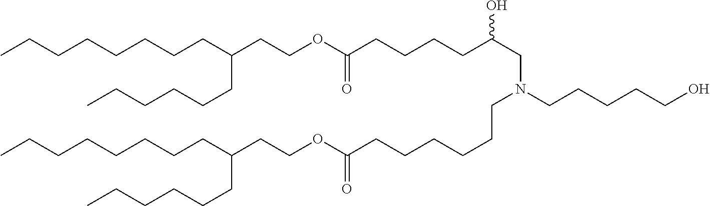 [SMILES: CCCCCCCCC(CCCCCC)CCOC(=O)CCCCCCN(CCCCCO)CC(O)CCCCC(=O)OCCC(CCCCCC)CCCCCCCC] |
| 136 | 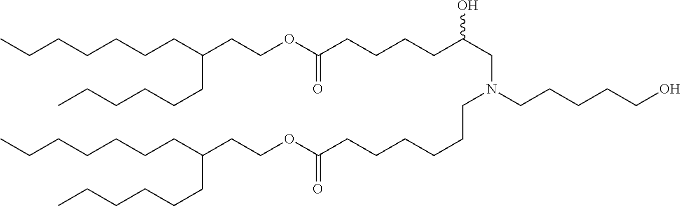 [SMILES: CCCCCCCC(CCCCCC)CCOC(=O)CCCCCCN(CCCCCO)CC(O)CCCCC(=O)OCCC(CCCCCC)CCCCCCC] |
| 137 | 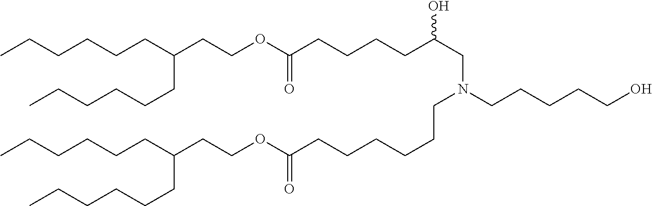 [SMILES: CCCCCCC(CCCCCC)CCOC(=O)CCCCCCN(CCCCCO)CC(O)CCCCC(=O)OCCC(CCCCCC)CCCCCC] |
| 138 | 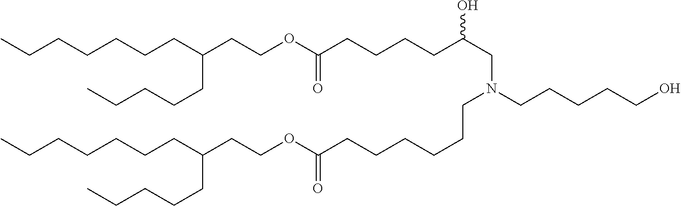 [SMILES: CCCCCCCC(CCCCC)CCOC(=O)CCCCCCN(CCCCCO)CC(O)CCCCC(=O)OCCC(CCCCC)CCCCCCC] |
| 139 | 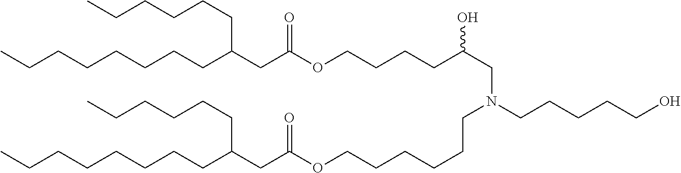 [SMILES: CCCCCCCCC(CCCCCC)CC(=O)OCCCCCCN(CCCCCO)CC(O)CCCCOC(=O)CC(CCCCCC)CCCCCCCC] |
| 140 | 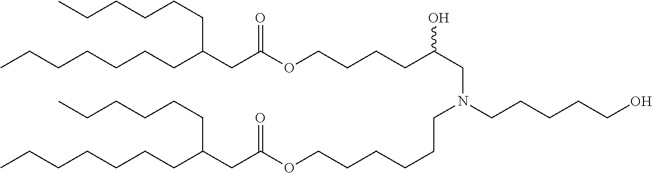 [SMILES: CCCCCCCC(CCCCCC)CC(=O)OCCCCCCN(CCCCCO)CC(O)CCCCOC(=O)CC(CCCCCC)CCCCCCC] |
| 141 | 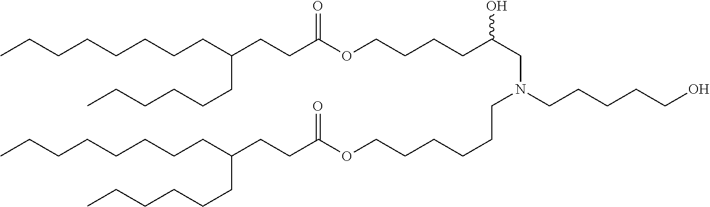 [SMILES: CCCCCCCCC(CCCCCC)CCC(=O)OCCCCCCN(CCCCCO)CC(O)CCCCOC(=O)CCC(CCCCCC)CCCCCCCC] |
| 142 | 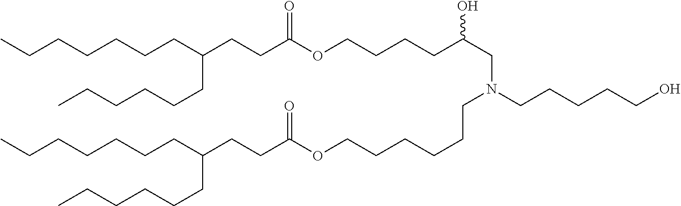 [SMILES: CCCCCCCC(CCCCCC)CCC(=O)OCCCCCCN(CCCCCO)CC(O)CCCCOC(=O)CCC(CCCCCC)CCCCCCC] |
| 143 | 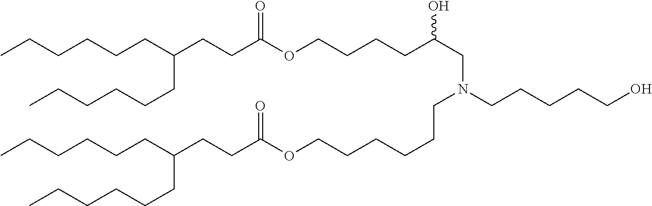 [SMILES: CCCCCCC(CCCCCC)CCC(=O)OCCCCCCN(CCCCCO)CC(O)CCCCOC(=O)CCC(CCCCCC)CCCCCC] |
| 144 | 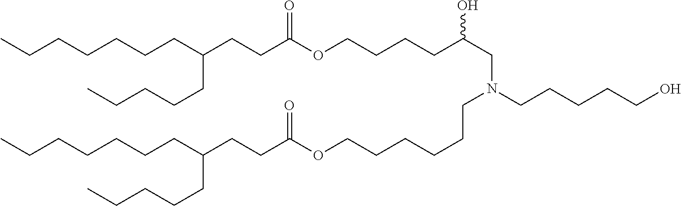 [SMILES: CCCCCCCC(CCCCC)CCC(=O)OCCCCCCN(CCCCCO)CC(O)CCCCOC(=O)CCC(CCCCC)CCCCCCC] |
| 145 | 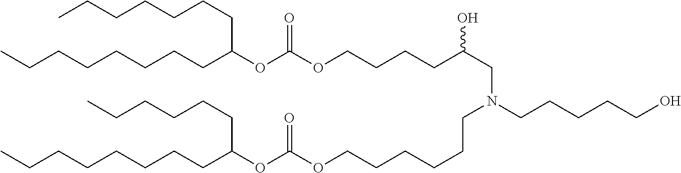 [SMILES: CCCCCCCCC(CCCCCC)OC(=O)OCCCCCCN(CCCCCO)CC(O)CCCCOC(=O)OC(CCCCCCC)CCCCCCCC] |
| 146 | 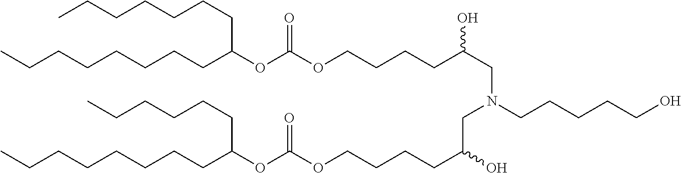 [SMILES: CCCCCCCCC(CCCCCC)OC(=O)OCCCCC(O)CN(CCCCCO)CC(O)CCCCOC(=O)OC(CCCCCCC)CCCCCCCC] |
| 147 | 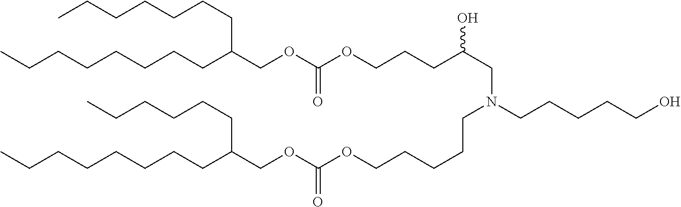 [SMILES: CCCCCCCCC(CCCCCC)COC(=O)OCCCCCN(CCCCCO)CC(O)CCCOC(=O)OCC(CCCCCCC)CCCCCCCC] |
| 148 | 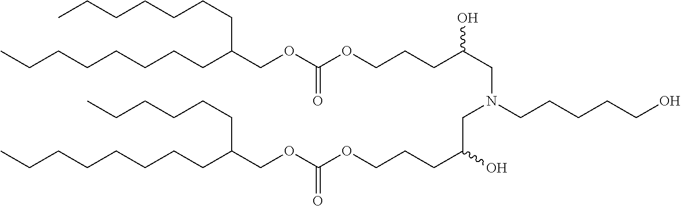 [SMILES: CCCCCCCCC(CCCCCC)COC(=O)OCCCC(O)CN(CCCCCO)CC(O)CCCOC(=O)OCC(CCCCCCC)CCCCCCCC] |
| 149 | 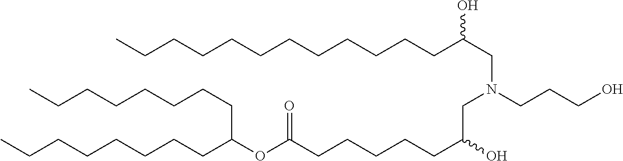 [SMILES: CCCCCCCCCCCCC(O)CN(CCCO)CC(O)CCCCCC(=O)OC(CCCCCCCC)CCCCCCCC] |
| 150 | 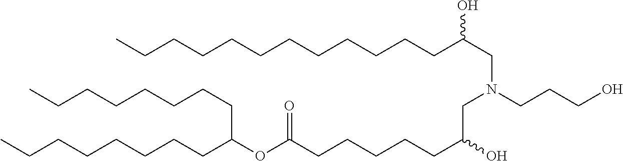 [SMILES: CCCCCCCCCCCCC(O)CN(CCCO)CC(O)CCCCCC(=O)OC(CCCCCCCC)CCCCCCCC] |
| 151 | 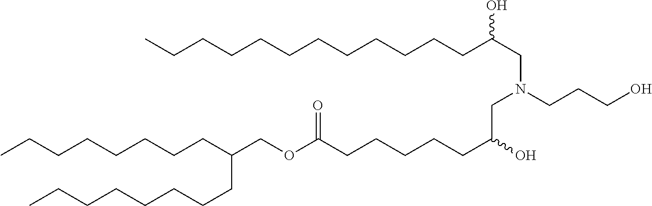 [SMILES: CCCCCCCCCCCCC(O)CN(CCCO)CC(O)CCCCCC(=O)OCC(CCCCCCCC)CCCCCCCC] |
| 152 | 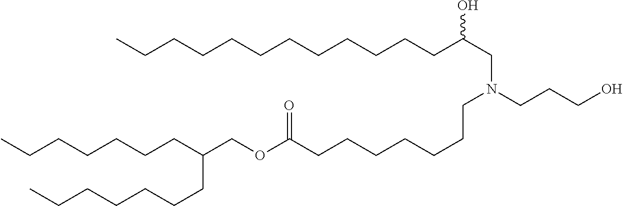 [SMILES: CCCCCCCCCCCCC(O)CN(CCCO)CCCCCCCC(=O)OCC(CCCCCCC)CCCCCCC] |
| 153 | 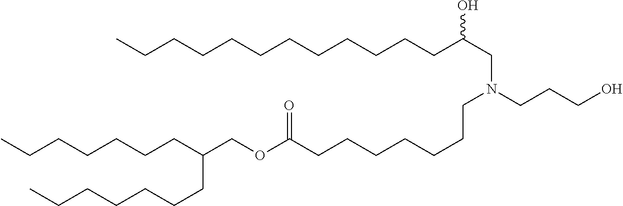 [SMILES: CCCCCCCCCCCCC(O)CN(CCCO)CCCCCCCC(=O)OCC(CCCCCCC)CCCCCCC] |
| 154 | 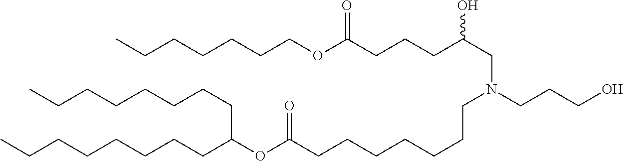 [SMILES: CCCCCCCCC(CCCCCCCC)OC(=O)CCCCCCCN(CCCO)CC(O)CCCC(=O)OCCCCCCC] |
| 155 | 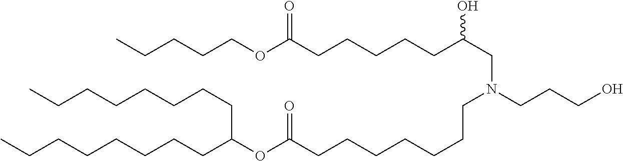 [SMILES: CCCCCCCCC(CCCCCCCC)OC(=O)CCCCCCCN(CCCO)CC(O)CCCCCC(=O)OCCCCC] |
| 156 | 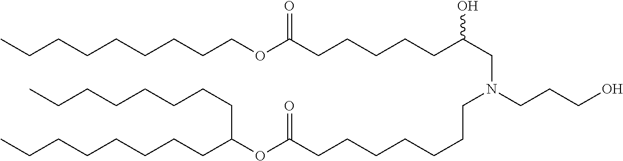 [SMILES: CCCCCCCCCOC(=O)CCCCCC(O)CN(CCCO)CCCCCCCC(=O)OC(CCCCCCCC)CCCCCCCC] |
| 157 | 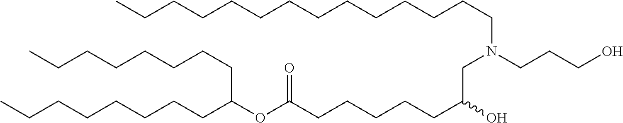 [SMILES: CCCCCCCCCCCCCCN(CCCO)CC(O)CCCCCC(=O)OC(CCCCCCCC)CCCCCCCC] |
| 158 | 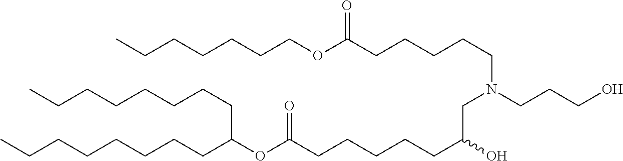 [SMILES: CCCCCCCCC(CCCCCCCC)OC(=O)CCCCCC(O)CN(CCCO)CCCCCC(=O)OCCCCCCC] |
| 159 | 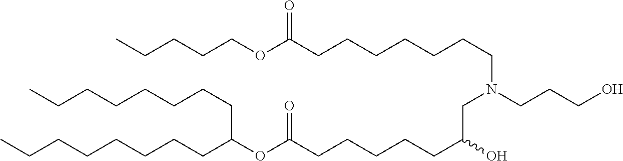 [SMILES: CCCCCCCCC(CCCCCCCC)OC(=O)CCCCCC(O)CN(CCCO)CCCCCCCC(=O)OCCCCC] |
| 160 | 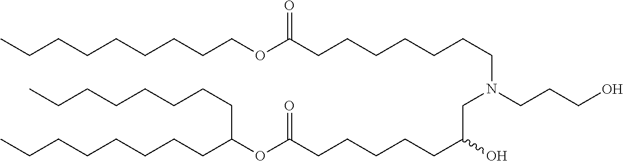 [SMILES: CCCCCCCCCOC(=O)CCCCCCCN(CCCO)CC(O)CCCCCC(=O)OC(CCCCCCCC)CCCCCCCC] |
| 161 | 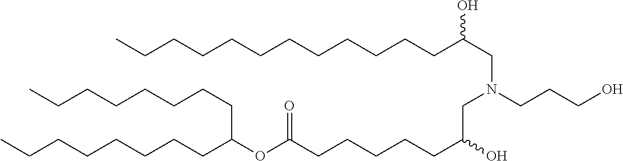 [SMILES: CCCCCCCCCCCCC(O)CN(CCCO)CC(O)CCCCCC(=O)OC(CCCCCCCC)CCCCCCCC] |
| 162 | 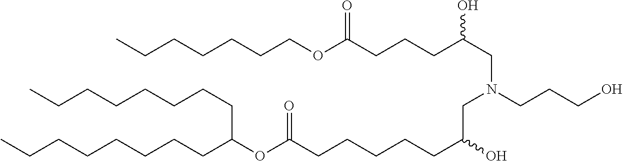 [SMILES: CCCCCCCCC(CCCCCCCC)OC(=O)CCCCCC(O)CN(CCCO)CC(O)CCCC(=O)OCCCCCCC] |
| 163 | 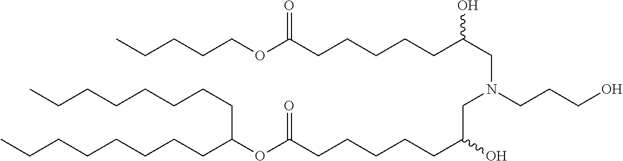 [SMILES: CCCCCCCCC(CCCCCCCC)OC(=O)CCCCCC(O)CN(CCCO)CC(O)CCCCCC(=O)OCCCCC] |
| 164 | 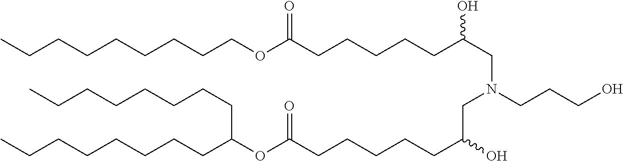 [SMILES: CCCCCCCCCOC(=O)CCCCCC(O)CN(CCCO)CC(O)CCCCCC(=O)OC(CCCCCCCC)CCCCCCCC] |
| 165 | 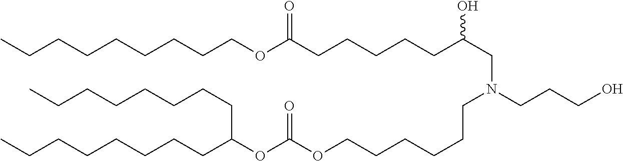 [SMILES: CCCCCCCCCOC(=O)CCCCCC(O)CN(CCCO)CCCCCCOC(=O)OC(CCCCCCCC)CCCCCCCC] |
| 166 | 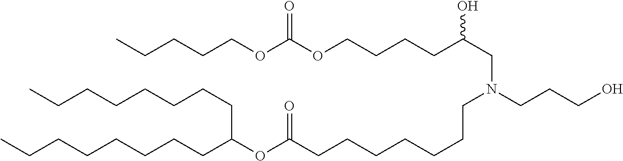 [SMILES: CCCCCCCCC(CCCCCCCC)OC(=O)CCCCCCCN(CCCO)CC(O)CCCCOC(=O)OCCCCC] |
| 167 | 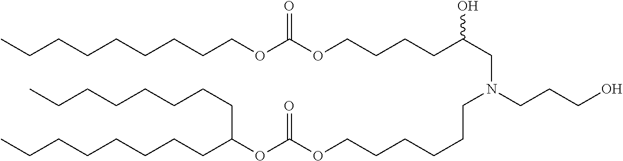 [SMILES: CCCCCCCCCOC(=O)OCCCCC(O)CN(CCCO)CCCCCCOC(=O)OC(CCCCCCCC)CCCCCCCC] |
| 168 | 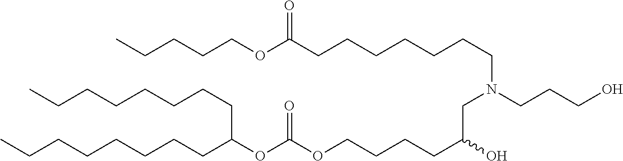 [SMILES: CCCCCCCCC(CCCCCCCC)OC(=O)OCCCCC(O)CN(CCCO)CCCCCCCC(=O)OCCCCC] |
| 169 | 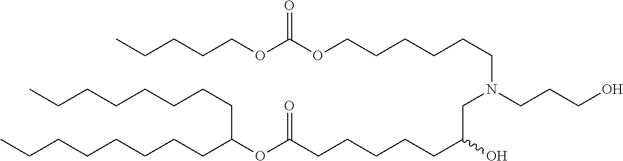 [SMILES: CCCCCCCCC(CCCCCCCC)OC(=O)CCCCCC(O)CN(CCCO)CCCCCCOC(=O)OCCCCC] |
| 170 | 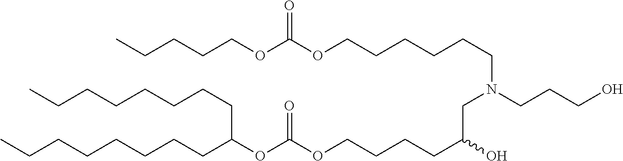 [SMILES: CCCCCCCCC(CCCCCCCC)OC(=O)OCCCCC(O)CN(CCCO)CCCCCCOC(=O)OCCCCC] |
| 171 | 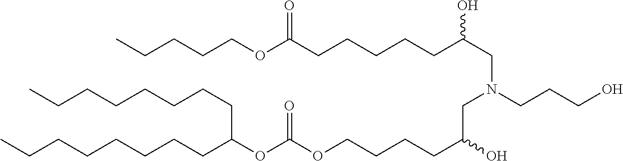 [SMILES: CCCCCCCCC(CCCCCCCC)OC(=O)OCCCCC(O)CN(CCCO)CC(O)CCCCCC(=O)OCCCCC] |
| 172 | 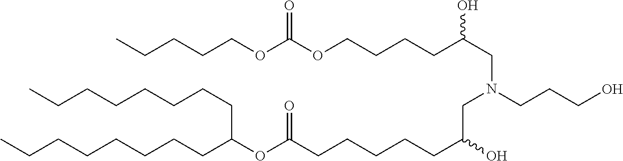 [SMILES: CCCCCCCCC(CCCCCCCC)OC(=O)CCCCCC(O)CN(CCCO)CC(O)CCCCOC(=O)OCCCCC] |
| 173 | 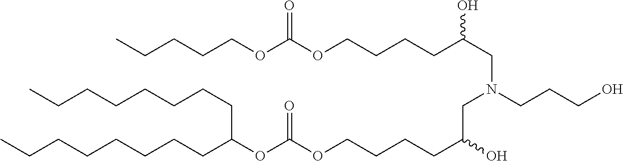 [SMILES: CCCCCCCCC(CCCCCCCC)OC(=O)OCCCCC(O)CN(CCCO)CC(O)CCCCOC(=O)OCCCCC] |
| 174 | 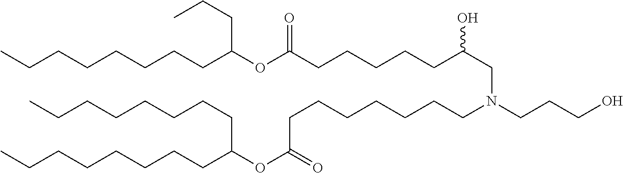 [SMILES: CCCCCCCCC(CCC)OC(=O)CCCCCC(O)CN(CCCO)CCCCCCCC(=O)OC(CCCCCCCC)CCCCCCCC] |
| 175 | 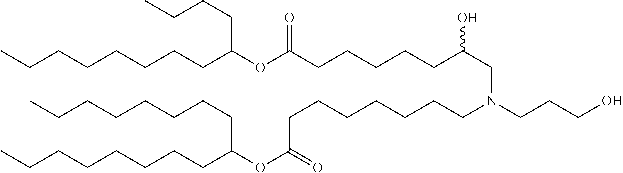 [SMILES: CCCCCCCCC(CCCC)OC(=O)CCCCCC(O)CN(CCCO)CCCCCCCC(=O)OC(CCCCCCCC)CCCCCCCC] |
| 176 | 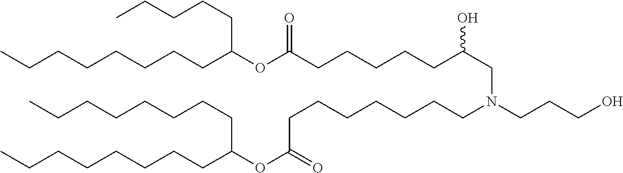 [SMILES: CCCCCCCCC(CCCCC)OC(=O)CCCCCC(O)CN(CCCO)CCCCCCCC(=O)OC(CCCCCCCC)CCCCCCCC] |
| 177 | 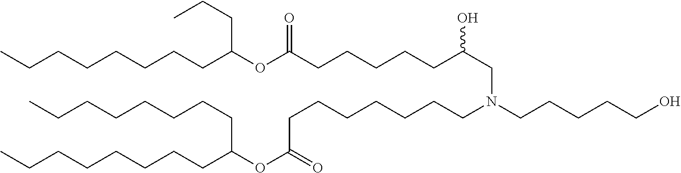 [SMILES: CCCCCCCCC(CCC)OC(=O)CCCCCC(O)CN(CCCCCO)CCCCCCCC(=O)OC(CCCCCCCC)CCCCCCCC] |
| 178 | 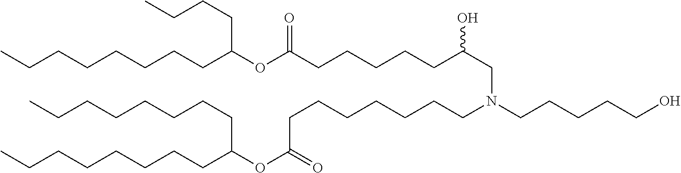 [SMILES: CCCCCCCCC(CCCC)OC(=O)CCCCCC(O)CN(CCCCCO)CCCCCCCC(=O)OC(CCCCCCCC)CCCCCCCC] |
| 179 | 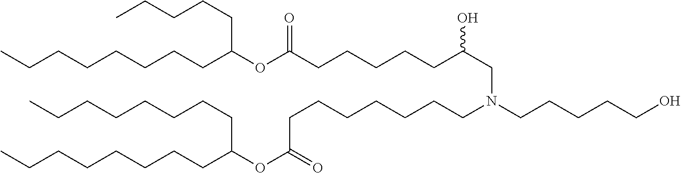 [SMILES: CCCCCCCCC(CCCCC)OC(=O)CCCCCC(O)CN(CCCCCO)CCCCCCCC(=O)OC(CCCCCCCC)CCCCCCCC] |
| 180 | 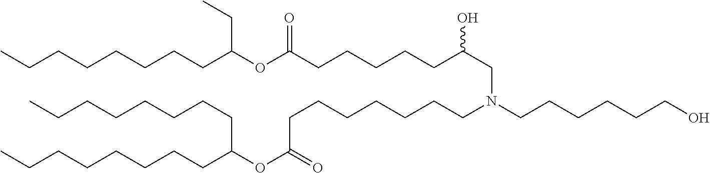 [SMILES: CCCCCCCCC(CC)OC(=O)CCCCCC(O)CN(CCCCCCO)CCCCCCCC(=O)OC(CCCCCCCC)CCCCCCCC] |
| 181 | 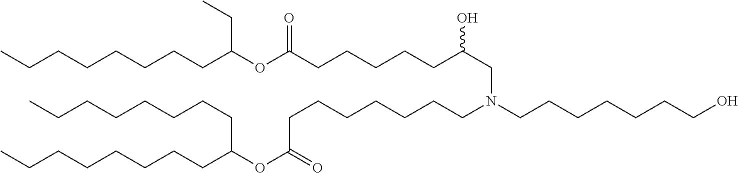 [SMILES: CCCCCCCCC(CC)OC(=O)CCCCCC(O)CN(CCCCCCCO)CCCCCCCC(=O)OC(CCCCCCCC)CCCCCCCC] |
| 182 | 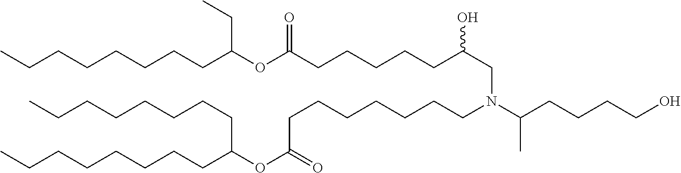 [SMILES: CCCCCCCCC(CC)OC(=O)CCCCCC(O)CN(CCCCCCCC(=O)OC(CCCCCCCC)CCCCCCCC)C(C)CCCCO] |
| 183 | 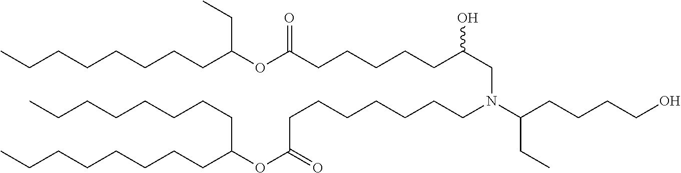 [SMILES: CCCCCCCCC(CC)OC(=O)CCCCCC(O)CN(CCCCCCCC(=O)OC(CCCCCCCC)CCCCCCCC)C(CC)CCCCO] |
| 184 | 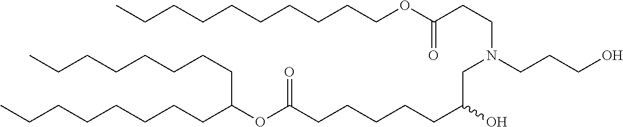 [SMILES: CCCCCCCCCCOC(=O)CCN(CCCO)CC(O)CCCCCC(=O)OC(CCCCCCCC)CCCCCCCC] |
| 185 | 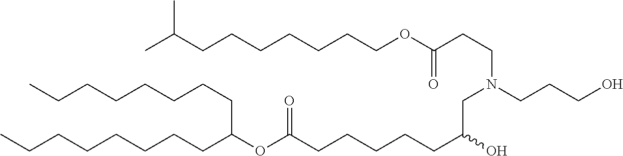 [SMILES: CCCCCCCCC(CCCCCCCC)OC(=O)CCCCCC(O)CN(CCCO)CCC(=O)OCCCCCCCC(C)C] |

In some embodiments, an ionizable lipid is a compound of Formula (15):

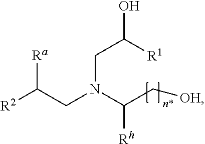

or is a pharmaceutically acceptable salt thereof, wherein:
n* is an integer from 1 to 7;
R^{a} is hydrogen or hydroxyl;
R^{h} is hydrogen or C₁-C₆ alkyl;
R¹ is C₁-C₃₀ alkyl or R^{1*};
R² is C₁-C₃₀ alkyl or R^{2*}.
R^{1*} and R^{2*} are independently selected from:

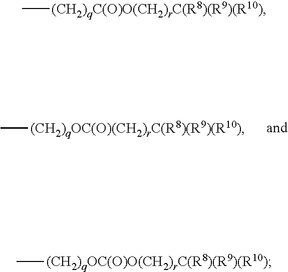

wherein:
   q is an integer from 0 to 12,
   r is an integer from 0 to 6, wherein at least one occurrence of r is not 0;
   R⁸ is H or R¹¹;
   R⁹, R¹⁰, and R¹¹ are each independently C₁-C₂₀ alkyl or C₂-C₂₀-alkenyl; and wherein (i) R¹ is R^{1*}, (ii) R² is R^{2*}, or (iii) R¹ is R^{1*} and R² is R^{2*}.

In some embodiments of Formula (15), R is hydrogen and the ionizable lipid is of Formula (16):

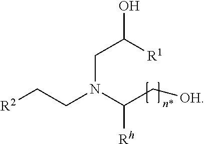

or is a pharmaceutically acceptable salt thereof, wherein:
n* is an integer from 1 to 7.

In some embodiments of Formula (16), the ionizable lipid is of Formula (17):

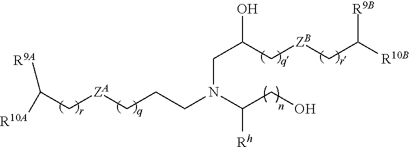

or a pharmaceutically acceptable salt thereof, wherein:
   n is an integer from 1 to 7;
   q and q′ are each independently integers from 0 to 12;
   r and r′ are each independently integers from 0 to 6, wherein at least one of r or r′ is not 0;
   Z^{A} and Z^{B} are each independently selected from \hat{ }—C(O)O—, \hat{ }—OC(O), and —OC(O)O—; where \hat{ } denotes the attachment point to —(CH₂)_{q}— or —(CH₂)_{q′}—; and
R^{9A}, R^{9B}, R^{10A}, and R^{10B} are each independently C₁-C₂₀ alkyl or C₂-C₂₀ alkenyl.

In some embodiments of Formula (17), Z^{A} and Z^{B} are \hat{ }—C(O)O—, and the ionizable lipid is of Formula (17a-1)

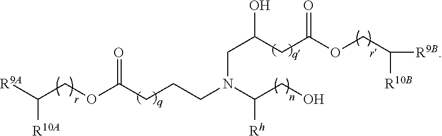

In some embodiments of Formula (17), Z^{A} and Z^{B} are \hat{ }—OC(O)—, and the ionizable lipid is of Formula (17a-2)

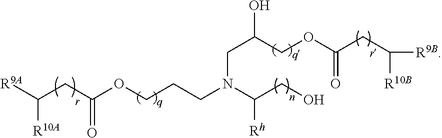

In some embodiments of Formula (17), Z^{A} and Z^{B} are —O(C)(O)O—, and the ionizable lipid is represented by Formula (17a-3):

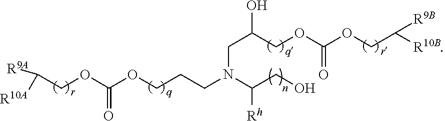

In some embodiments of Formula (15), R^{a} is hydroxyl and the ionizable lipid is of Formula (18):

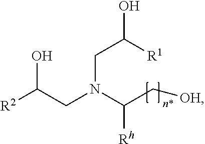

or is a pharmaceutically acceptable salt thereof, wherein:
n* is an integer from 1 to 7;
R^{h} is hydrogen or C₁-C₆ alkyl;
R¹ is C₁-C₃₀ alkyl or R^{1*},
R² is C₁-C₃₀ alkyl or R^{2*};
R^{1*} and R^{2*} are independently selected from:

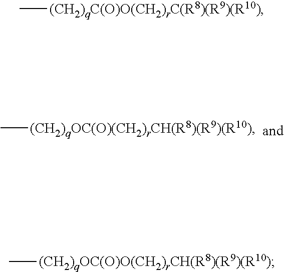

wherein:
   q is an integer from 0 to 12,
   r is an integer from 0 to 6, wherein at least one occurrence of r is not 0;
   R⁸ is hydrogen or R¹¹;
   R⁹, R¹⁰, and R¹¹ are each independently C₁-C₂₀ alkyl or C₂-C₂₀-alkenyl;
wherein (i) R¹ is R^{1*}, (ii) R² is R^{2*}, or (iii) R¹ is R^{1*} and R² is R^{2*}; and
wherein, for (iii), (a) R^{1*} and R^{2*} are different or (b) R⁹ and R¹⁰ have different numbers of carbon atoms for at least one of R^{1*} and R^{2*}.

In some embodiments of Formula (18), the ionizable lipid of is of Formula (19):

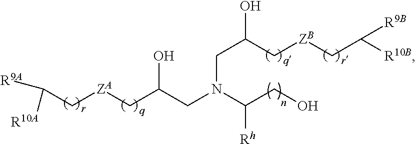

or is a pharmaceutically acceptable salt thereof, wherein:
n is an integer from 1 to 7;
q and q′ are each independently integers from 0 to 12;
r and r′ are each independently integers from 0 to 6, wherein at least one of r or r′ is not 0;
Z^{A} and Z^{B} are each independently selected from \hat{ }—C(O)O—, \hat{ }—OC(O), and —OC(O)O—; where \hat{ } denotes the attachment point to —(CH₂)_{q}— or —(CH₂)_{q′}; and
R^{9A}, R^{9B}, R^{10A}, and R^{10B} are each independently C₁-C₂₀ alkyl or C₂-C₂₀ alkenyl.

In some embodiments of Formula (19), Z^{A} and Z^{B} are \hat{ }—C(O)O—, and the ionizable lipid is of Formula (19a-1):

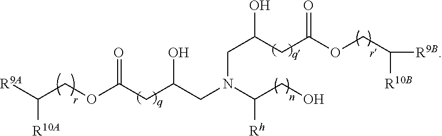

In some embodiments of Formula (19), Z^{A} and Z^{B} are \hat{ }—OC(O)—, and the ionizable lipid is of Formula (19a-2):

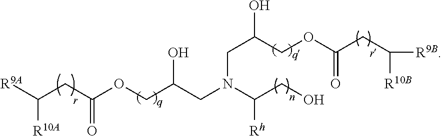

In some embodiments of Formula (19), Z^{A} and Z^{B} are —O(C)(O)O—, and the ionizable lipid is represented by Formula (19a-3):

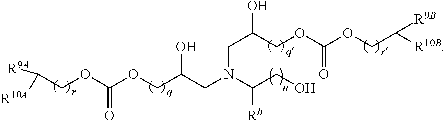

In some embodiments of Formula (15), R¹ is C₁-C₃₀ alkyl, and the ionizable lipid is of Formula (20):

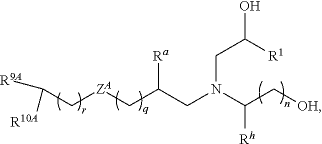

or is a pharmaceutically acceptable salt thereof, wherein:
Z^{A} is selected from \hat{ }—C(O)O—, \hat{ }—OC(O)—, and —OC(O)O—; where \hat{ } denotes the attachment point to —(CH₂)_{q}—;
R^{9A} and R^{10A} are each independently C₁-C₂₀ alkyl or C₂-C₂₀ alkenyl;
n is an integer from 1 to 7;
q is an integer from 0 to 12; and
r is an integer from 1 to 6.
In some embodiments of Formula (20), Z^{A} is \hat{ }—C(O)O—, and the ionizable lipid is of Formula (20a-1):

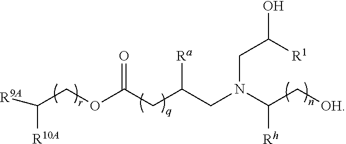

In some embodiments of Formula (20), Z^{A} is \hat{ }—OC(O)—, and the ionizable lipid is of Formula (20a-2):

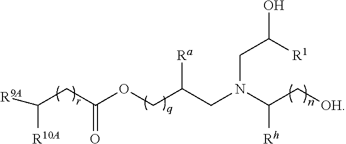

In some embodiments of Formula (20), Z^{A} is —OC(O)O—, and the ionizable lipid is of Formula (20a-3):

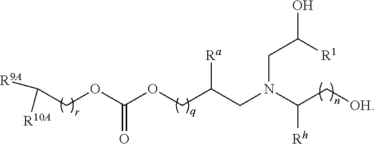

In some embodiments of Formula (15), R² is C₁-C₃₀ alkyl, and the ionizable lipid is of Formula (21):

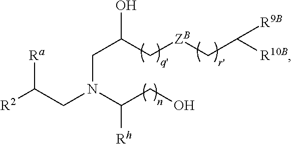

or is a pharmaceutically acceptable salt thereof, wherein:
Z^{B} is selected from \hat{ }—C(O)O—, \hat{ }—OC(O)—, and —OC(O)O—; where \hat{ } denotes the attachment point to —(CH₂)_{q′}—;
R^{9B} and R^{10B} are each independently C₁-C₂₀ alkyl or C₂-C₂₀ alkenyl;
n is an integer from 1 to 7;
q′ is an integer from 0 to 12; and
r′ is an integer from 1 to 6.

In some embodiments of Formula (21), Z^{B} is \hat{ }—C(O)O— and the ionizable lipid is of Formula (21a-1):

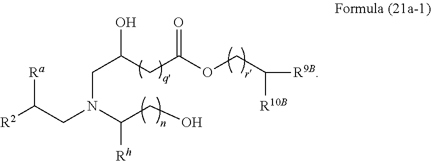

In some embodiments of Formula (21), Z^{B} is \hat{ }—OC(O)— and the ionizable lipid is of Formula (21a-2):

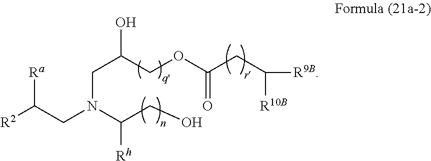

In some embodiments of Formula (21), Z^{B} is —OC(O)O—, and the ionizable lipid is of Formula (21a-3):

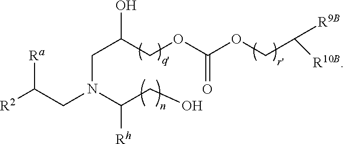

In some embodiments, an ionizable lipid is selected from the table below:

| Ionizable | |
|---|---|
| lipid | |
| number | Structure |
| 1-a | 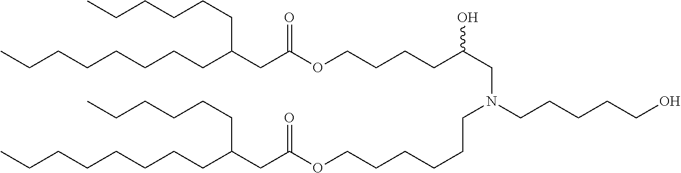 [SMILES: CCCCCCCCC(CCCCCC)CC(=O)OCCCCCCN(CCCCCO)CC(O)CCCCOC(=O)CC(CCCCCC)CCCCCCCC] |
| 1-b | 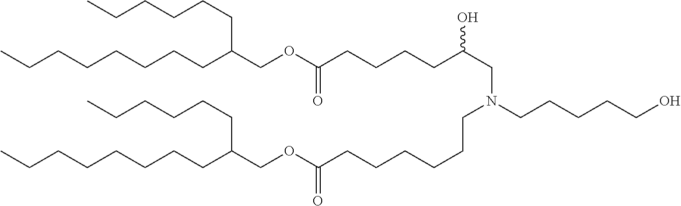 [SMILES: CCCCCCCCC(CCCCCC)COC(=O)CCCCCCN(CCCCCO)CC(O)CCCCC(=O)OCC(CCCCCC)CCCCCCCC] |
| 1-c | 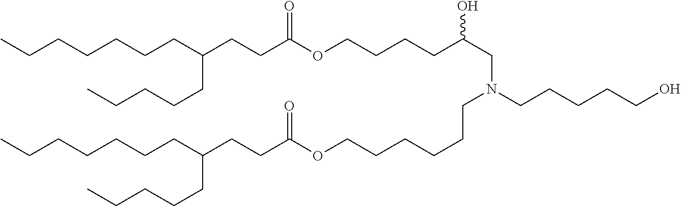 [SMILES: CCCCCCCC(CCCCC)CCC(=O)OCCCCCCN(CCCCCO)CC(O)CCCCOC(=O)CCC(CCCCC)CCCCCCC] |
| 1-d | 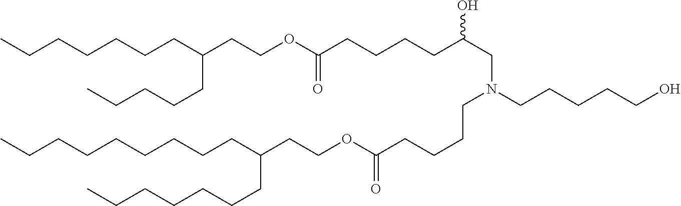 [SMILES: CCCCCCCCCC(CCCCCCC)CCOC(=O)CCCCN(CCCCCO)CC(O)CCCCC(=O)OCCC(CCCCC)CCCCCCC] |
| 1-e | 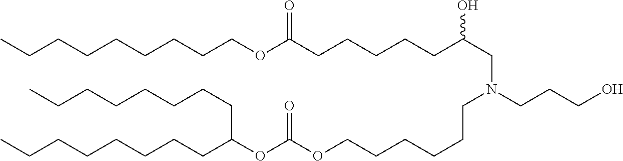 [SMILES: CCCCCCCCCOC(=O)CCCCCC(O)CN(CCCO)CCCCCCOC(=O)OC(CCCCCCCC)CCCCCCCC] |
| 1-f | 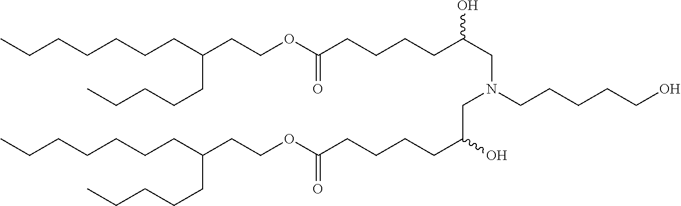 [SMILES: CCCCCCCC(CCCCC)CCOC(=O)CCCCC(O)CN(CCCCCO)CC(O)CCCCC(=O)OCCC(CCCCC)CCCCCCC] |
| 1-g | 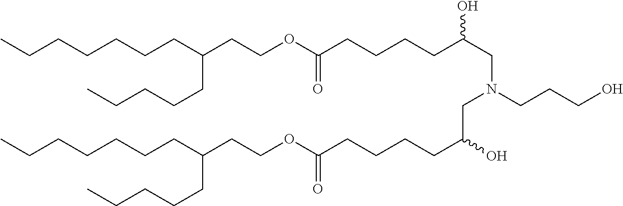 [SMILES: CCCCCCCC(CCCCC)CCOC(=O)CCCCC(O)CN(CCCO)CC(O)CCCCC(=O)OCCC(CCCCC)CCCCCCC] |
| 1-h | 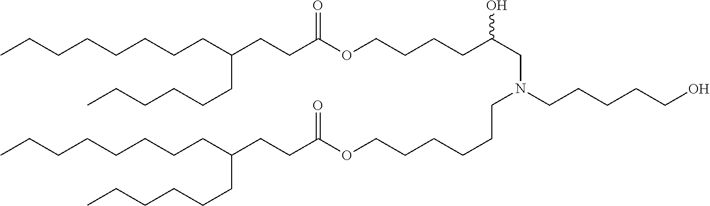 [SMILES: CCCCCCCCC(CCCCCC)CCC(=O)OCCCCCCN(CCCCCO)CC(O)CCCCOC(=O)CCC(CCCCCC)CCCCCCCC] |
| 1-i | 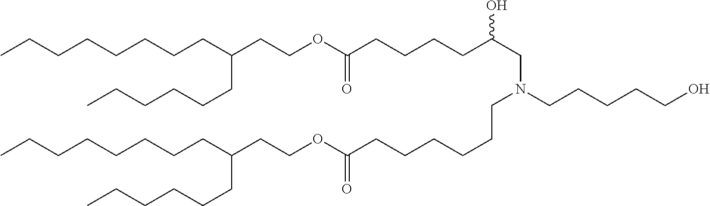 [SMILES: CCCCCCCCC(CCCCCC)CCOC(=O)CCCCCCN(CCCCCO)CC(O)CCCCC(=O)OCCC(CCCCCC)CCCCCCCC] |
| 1-j | 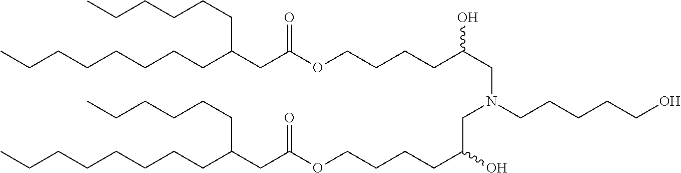 [SMILES: CCCCCCCCC(CCCCCC)CC(=O)OCCCCC(O)CN(CCCCCO)CC(O)CCCCOC(=O)CC(CCCCCC)CCCCCCCC] |
| 1-k | 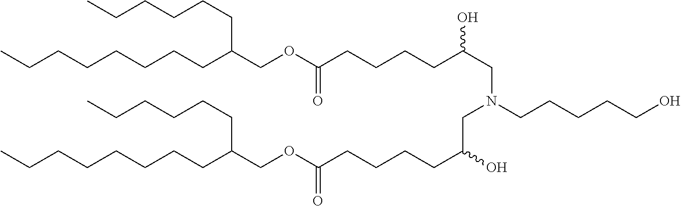 [SMILES: CCCCCCCCC(CCCCCC)COC(=O)CCCCC(O)CN(CCCCCO)CC(O)CCCCC(=O)OCC(CCCCCC)CCCCCCCC] |
| 1-l | 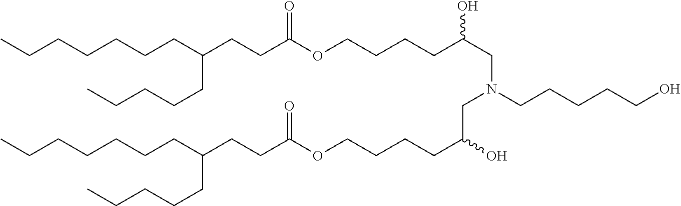 [SMILES: CCCCCCCC(CCCCC)CCC(=O)OCCCCC(O)CN(CCCCCO)CC(O)CCCCOC(=O)CCC(CCCCC)CCCCCCC] |
| 1-m | 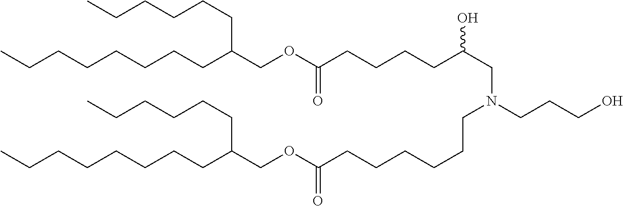 [SMILES: CCCCCCCCC(CCCCCC)COC(=O)CCCCCCN(CCCO)CC(O)CCCCC(=O)OCC(CCCCCC)CCCCCCCC] |
| 1-m | 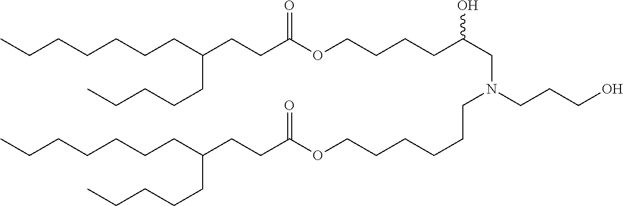 [SMILES: CCCCCCCC(CCCCC)CCC(=O)OCCCCCCN(CCCO)CC(O)CCCCOC(=O)CCC(CCCCC)CCCCCCC] |
| 1-n | 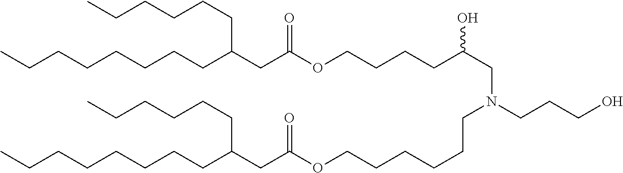 [SMILES: CCCCCCCCC(CCCCCC)CC(=O)OCCCCCCN(CCCO)CC(O)CCCCOC(=O)CC(CCCCCC)CCCCCCCC] |
| 1-o | 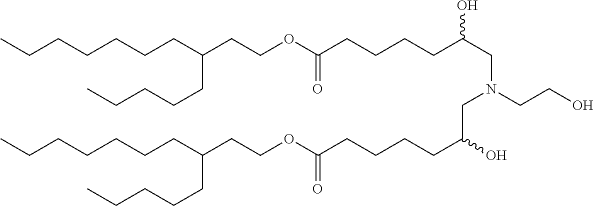 [SMILES: CCCCCCCC(CCCCC)CCOC(=O)CCCCC(O)CN(CCO)CC(O)CCCCC(=O)OCCC(CCCCC)CCCCCCC] |
| 1-p | 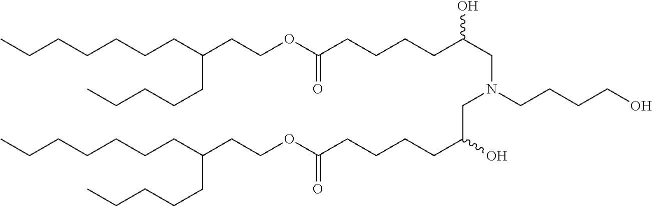 [SMILES: CCCCCCCC(CCCCC)CCOC(=O)CCCCC(O)CN(CCCCO)CC(O)CCCCC(=O)OCCC(CCCCC)CCCCCCC] |
| 1-q | 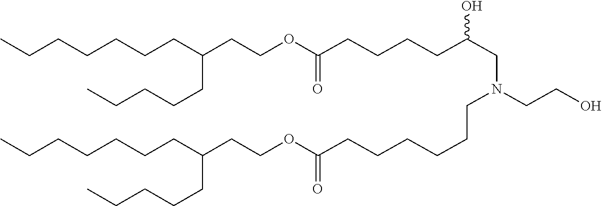 [SMILES: CCCCCCCC(CCCCC)CCOC(=O)CCCCCCN(CCO)CC(O)CCCCC(=O)OCCC(CCCCC)CCCCCCC] |
| 1-r | 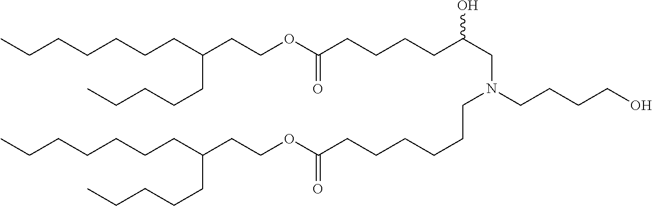 [SMILES: CCCCCCCC(CCCCC)CCOC(=O)CCCCCCN(CCCCO)CC(O)CCCCC(=O)OCCC(CCCCC)CCCCCCC] |
| 1-s | 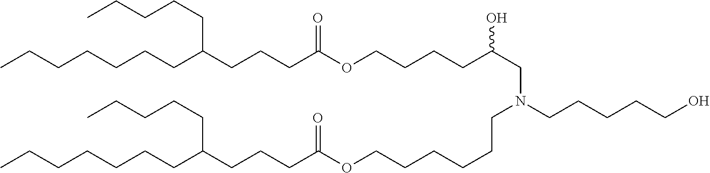 [SMILES: CCCCCCCC(CCCCC)CCCC(=O)OCCCCCCN(CCCCCO)CC(O)CCCCOC(=O)CCCC(CCCCC)CCCCCCC] |
| 1-t | 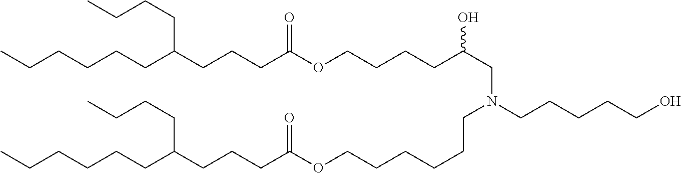 [SMILES: CCCCCCC(CCCC)CCCC(=O)OCCCCCCN(CCCCCO)CC(O)CCCCOC(=O)CCCC(CCCC)CCCCCC] |
| 1-u | 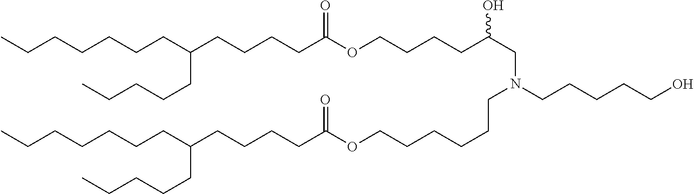 [SMILES: CCCCCCCC(CCCCC)CCCCC(=O)OCCCCCCN(CCCCCO)CC(O)CCCCOC(=O)CCCCC(CCCCC)CCCCCCC] |
| 1-v | 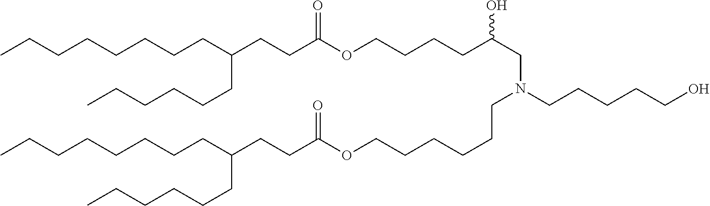 [SMILES: CCCCCCCCC(CCCCCC)CCC(=O)OCCCCCCN(CCCCCO)CC(O)CCCCOC(=O)CCC(CCCCCC)CCCCCCCC] |
| 1-w | 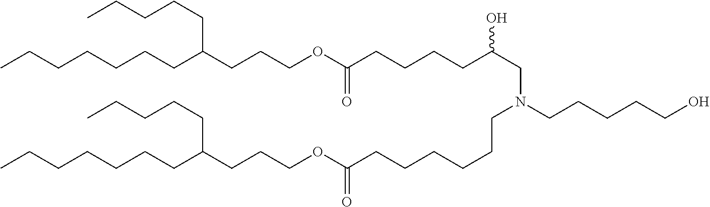 [SMILES: CCCCCCCC(CCCCC)CCCOC(=O)CCCCCCN(CCCCCO)CC(O)CCCCC(=O)OCCCC(CCCCC)CCCCCCC] |
| 1-x | 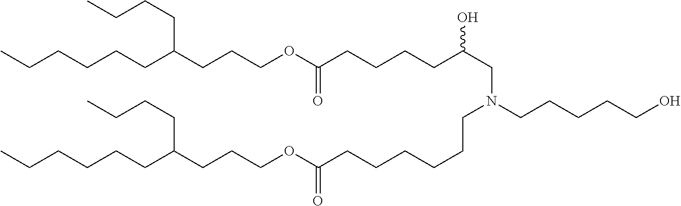 [SMILES: CCCCCCC(CCCC)CCCOC(=O)CCCCCCN(CCCCCO)CC(O)CCCCC(=O)OCCCC(CCCC)CCCCCC] |
| 1-y | 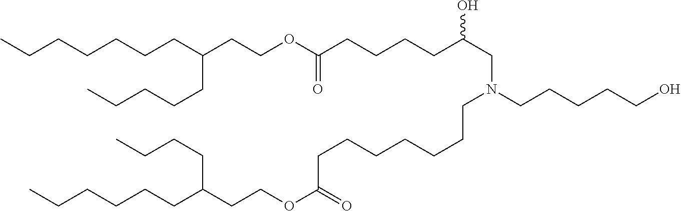 [SMILES: CCCCCCCC(CCCCC)CCOC(=O)CCCCC(O)CN(CCCCCO)CCCCCCCC(=O)OCCC(CCCC)CCCCCC] |
| 1-z | 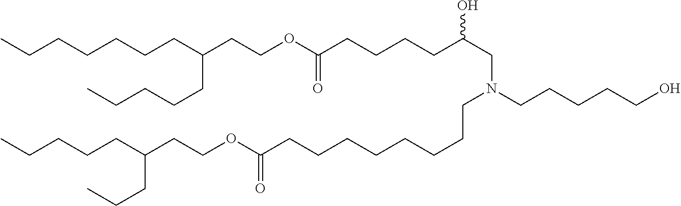 [SMILES: CCCCCCCC(CCCCC)CCOC(=O)CCCCC(O)CN(CCCCCO)CCCCCCCCC(=O)OCCC(CCC)CCCCC] |
| 1-aa | 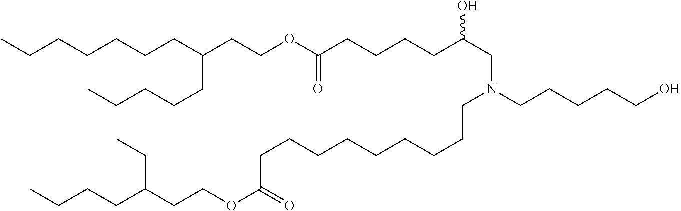 [SMILES: CCCCCCCC(CCCCC)CCOC(=O)CCCCC(O)CN(CCCCCO)CCCCCCCCCC(=O)OCCC(CC)CCCC] |
| 1-ab | 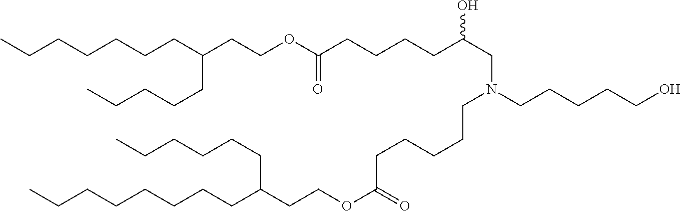 [SMILES: CCCCCCCCC(CCCCCC)CCOC(=O)CCCCCN(CCCCCO)CC(O)CCCCC(=O)OCCC(CCCCC)CCCCCCC] |
| 1-ac | 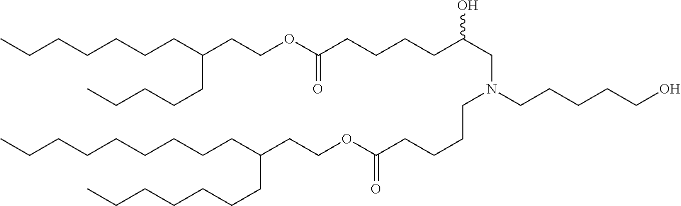 [SMILES: CCCCCCCCCC(CCCCCCC)CCOC(=O)CCCCN(CCCCCO)CC(O)CCCCC(=O)OCCC(CCCCC)CCCCCCC] |
| 1-ad | 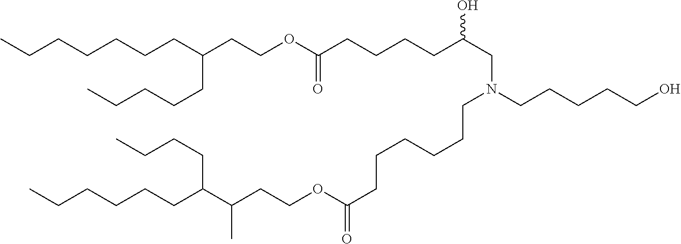 [SMILES: CCCCCCCC(CCCCC)CCOC(=O)CCCCC(O)CN(CCCCCO)CCCCCCC(=O)OCCC(C)C(CCCC)CCCCCC] |
| 1-ae | 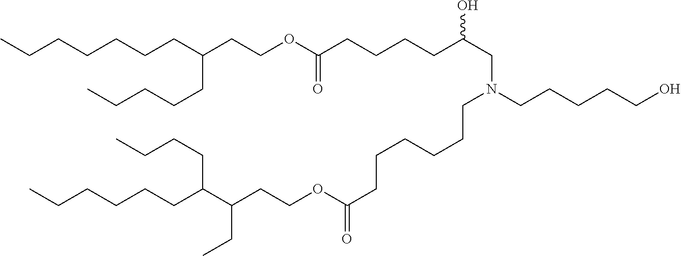 [SMILES: CCCCCCCC(CCCCC)CCOC(=O)CCCCC(O)CN(CCCCCO)CCCCCCC(=O)OCCC(CC)C(CCCC)CCCCCC] |
| 1-af | 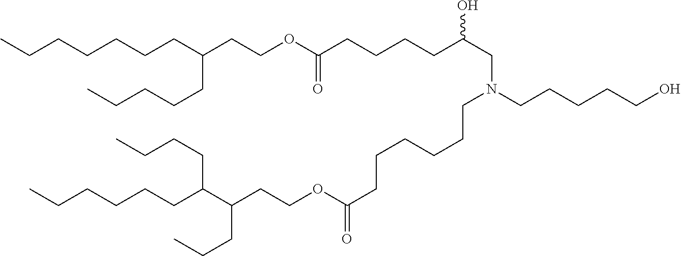 [SMILES: CCCCCCCC(CCCCC)CCOC(=O)CCCCC(O)CN(CCCCCO)CCCCCCC(=O)OCCC(CCC)C(CCCC)CCCCCC] |
| 1-ag | 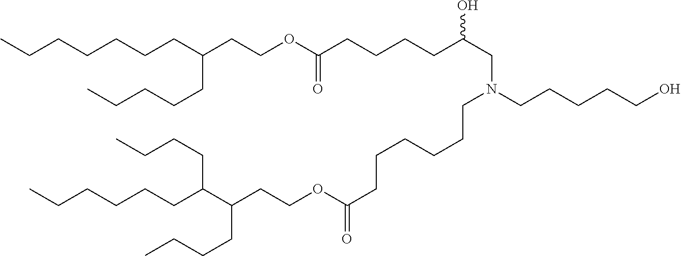 [SMILES: CCCCCCCC(CCCCC)CCOC(=O)CCCCC(O)CN(CCCCCO)CCCCCCC(=O)OCCC(CCCC)C(CCCC)CCCCCC] |
| 1-ah | 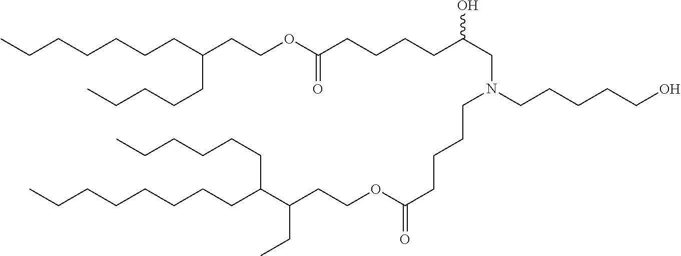 [SMILES: CCCCCCCCC(CCCCCC)C(CC)CCOC(=O)CCCCN(CCCCCO)CC(O)CCCCC(=O)OCCC(CCCCC)CCCCCCC] |
| 1-ai | 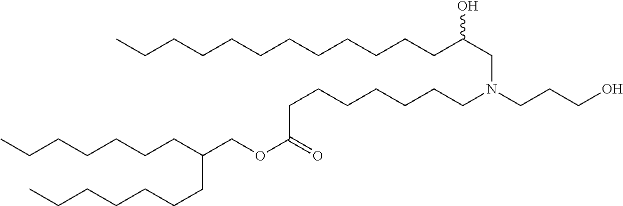 [SMILES: CCCCCCCCCCCCC(O)CN(CCCO)CCCCCCCC(=O)OCC(CCCCCCC)CCCCCCC] |
| 1-aj | 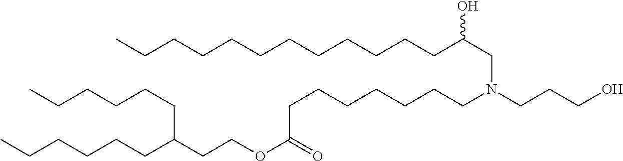 [SMILES: CCCCCCCCCCCCC(O)CN(CCCO)CCCCCCCC(=O)OCCC(CCCCCC)CCCCCC] |
| 1-ak | 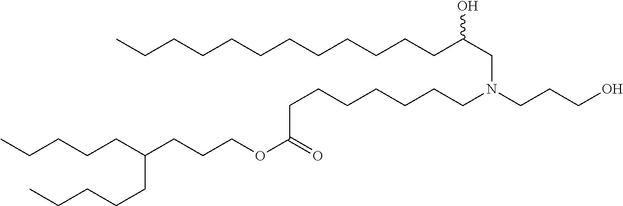 [SMILES: CCCCCCCCCCCCC(O)CN(CCCO)CCCCCCCC(=O)OCCCC(CCCCC)CCCCC] |

In some embodiments, an ionizable lipid of the present disclosure is represented by Formula (22):

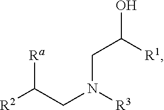

or is a pharmaceutically acceptable salt thereof, wherein:
R^{a} is hydrogen or hydroxyl;
R¹ is C₁-C₃₀ alkyl or R¹*;
R² is C₁-C₃₀ alkyl or R^{2*};
R^{1*} and R^{2*} are independently selected from:

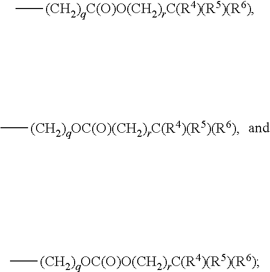

wherein:
   q is an integer from 0 to 12,
   r is an integer from 0 to 6, wherein at least one occurrence of r is not 0;
   R⁴ is hydrogen or R⁷;
   R⁵, R⁶, and R⁷ are each independently C₁-C₂₀ alkyl or C₂-C₂₀-alkenyl;
wherein (i) R¹ is R^{1*}, (ii) R² is R^{2*}, or (iii) R¹ is R^{1*} and R² is R^{2*}; and
R³ is L-R′, wherein L is linear or branched C₁-C₁₀ alkylene, and R′ is (i) mono- or bicyclic heterocyclyl or heteroaryl, such as imidazolyl, pyrazolyl, 1,2,4-triazolyl, or benzimidazolyl, each optionally substituted at one or more available carbon and nitrogen by C₁-C₆ alkyl, or (ii) R^{A}, R^{B}, or R^{C}, wherein
   R^{A} is selected from:

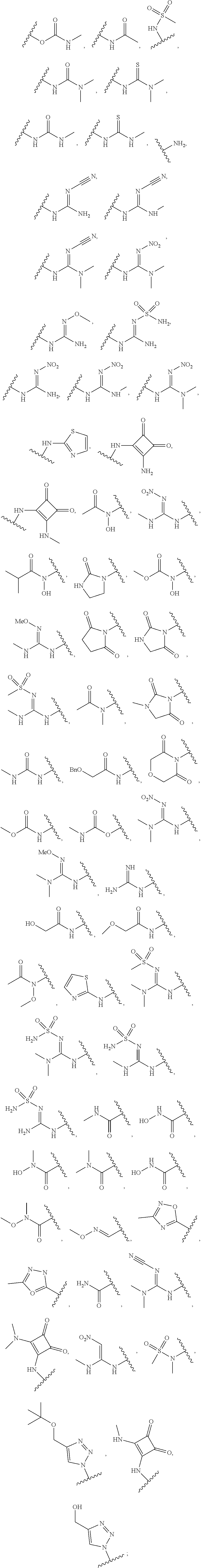

R^{B} is selected from:

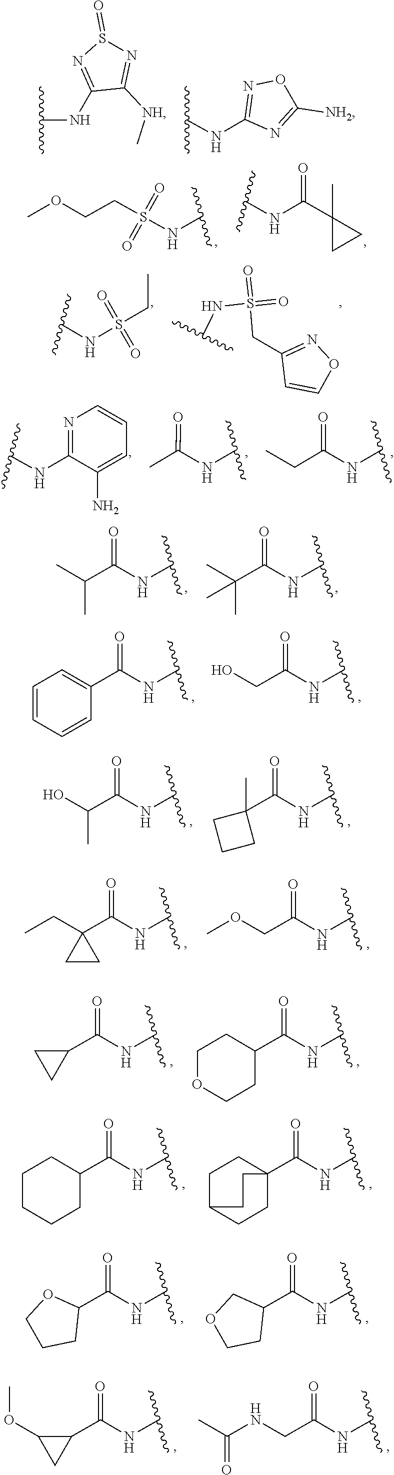

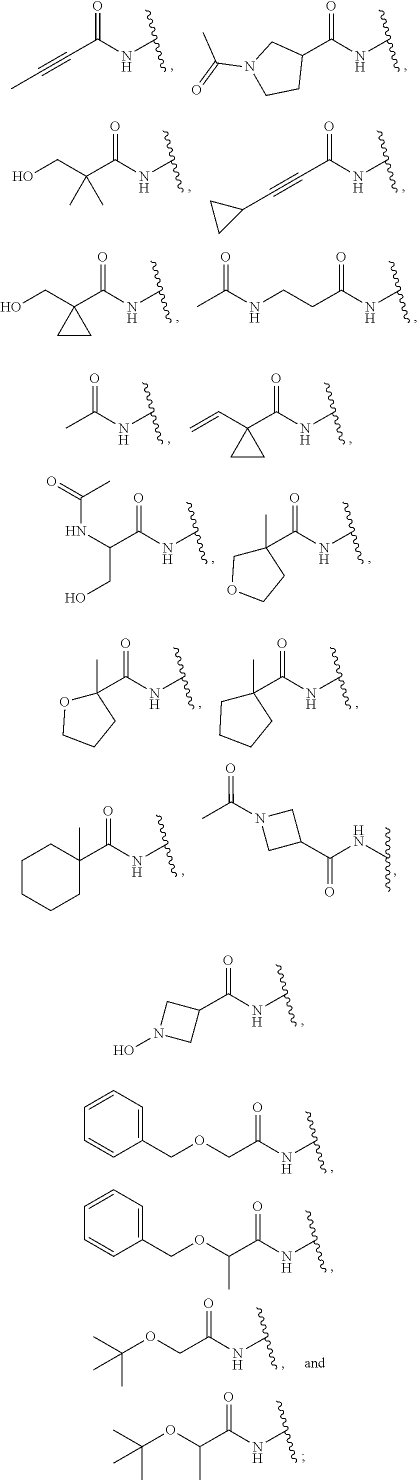

and
R^{C} is selected from:

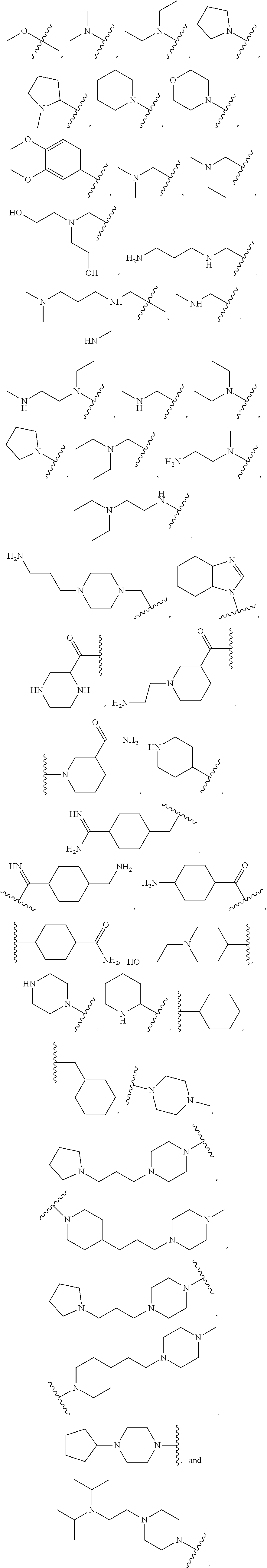

with the proviso that the ionizable lipid is not:

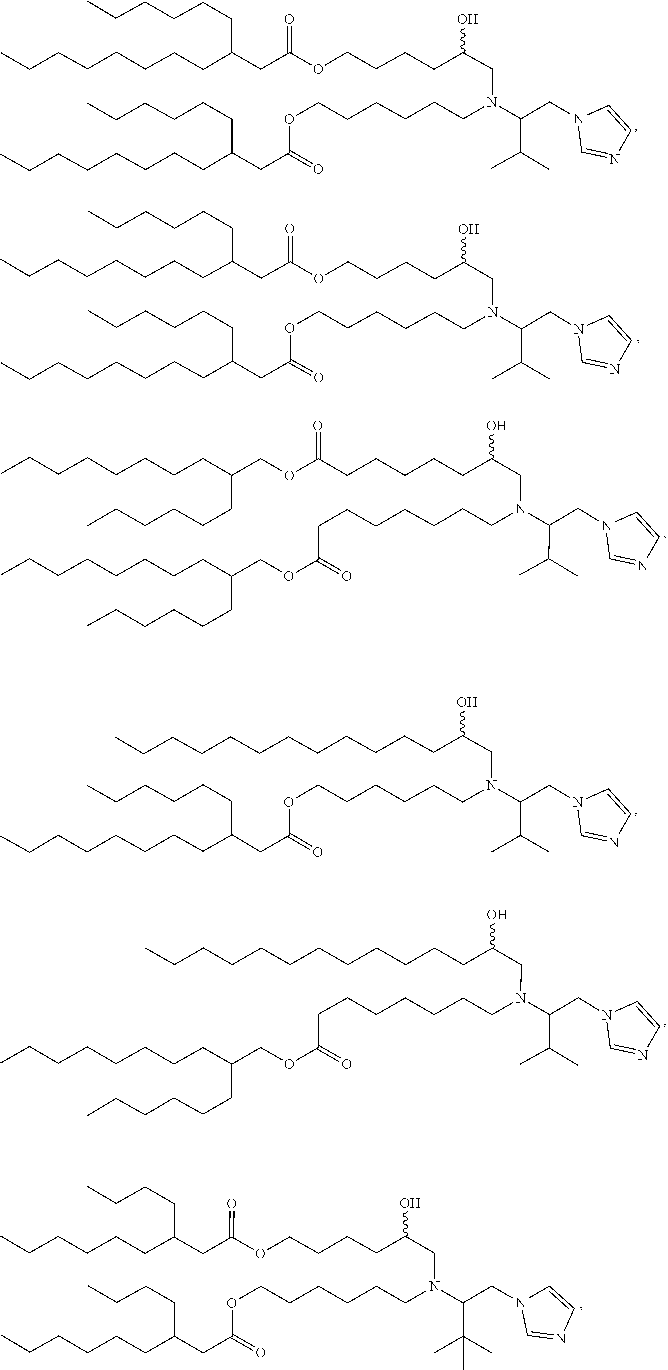

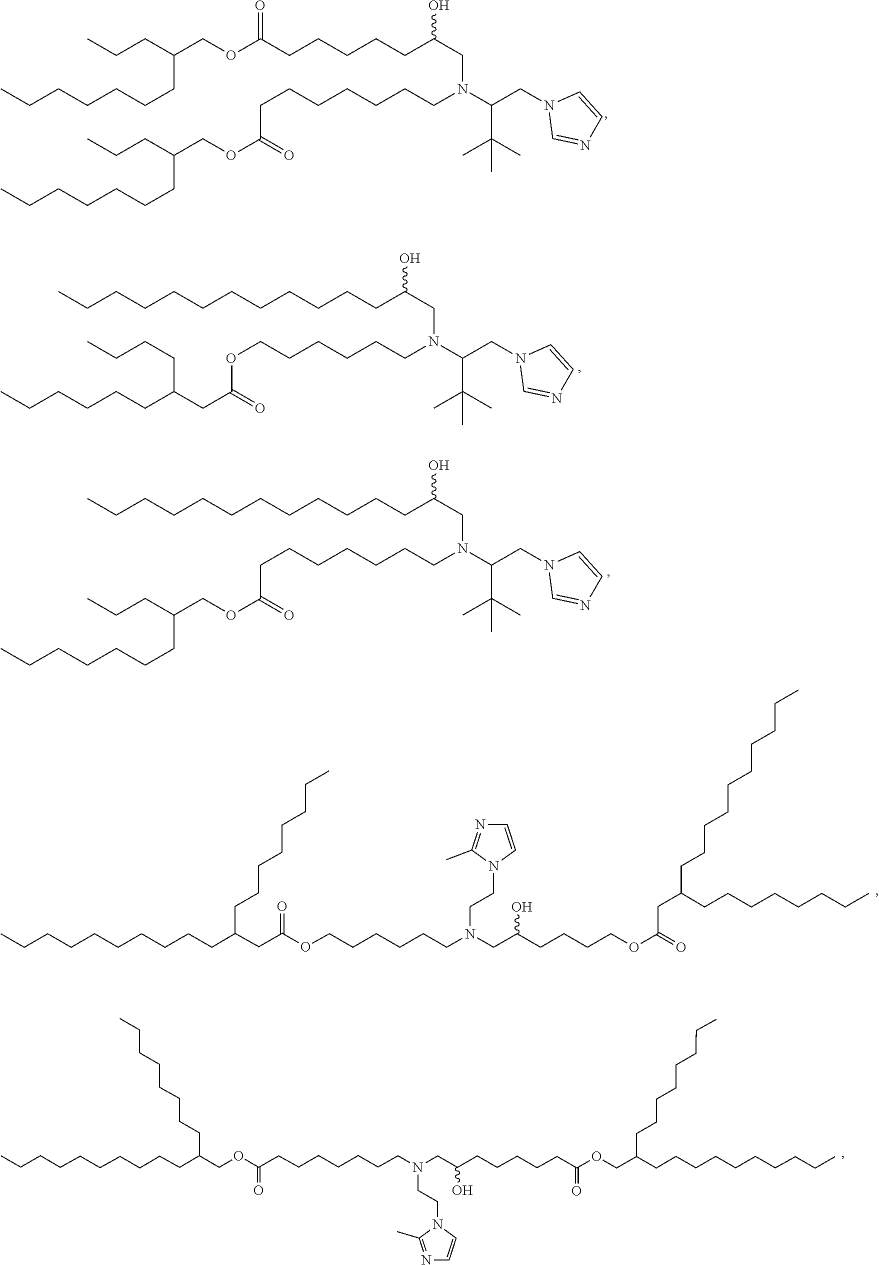

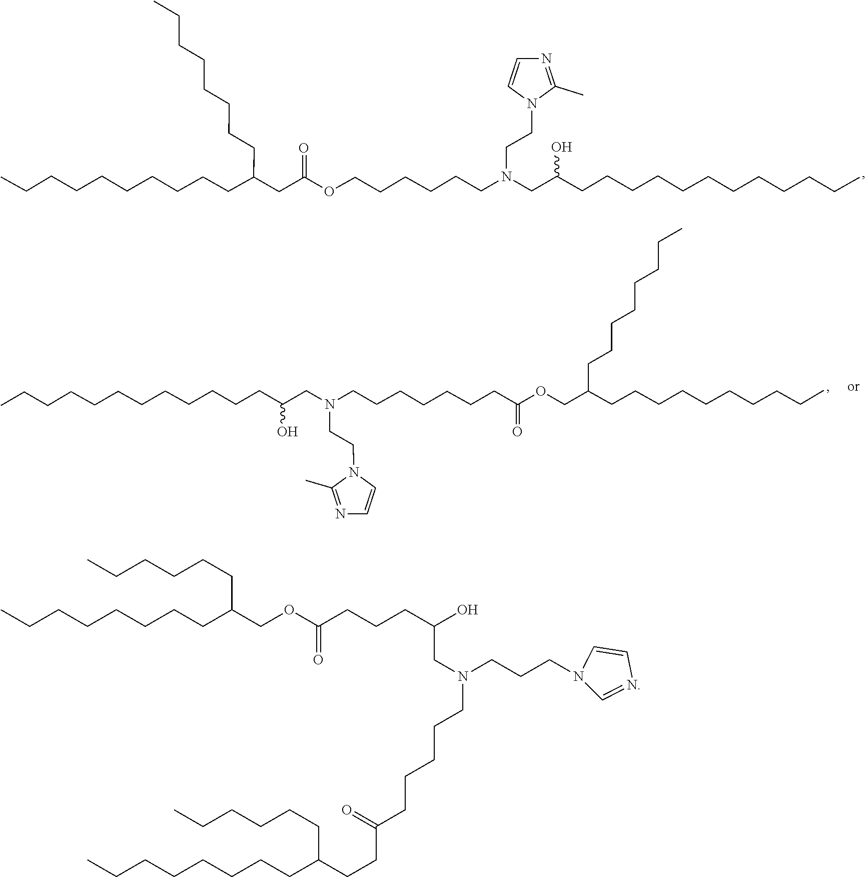

In some embodiments of Formula (22), R³ is selected from:

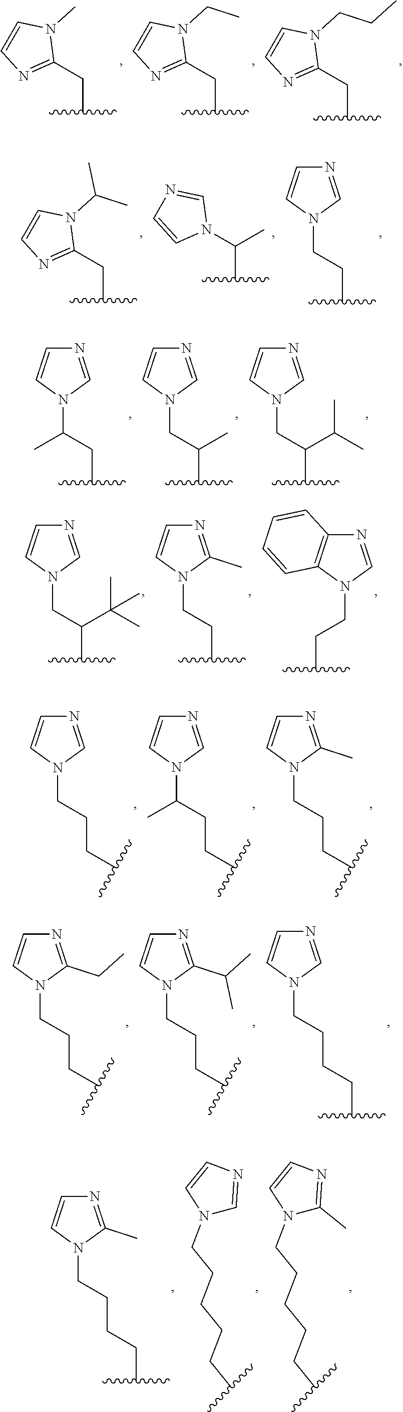

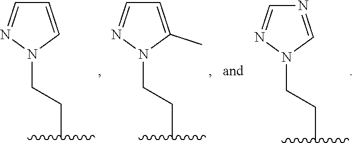

In some embodiments of Formula (22), R¹ is R^{1*}, R² is R^{2*}, and the ionizable lipid is of Formula (23):

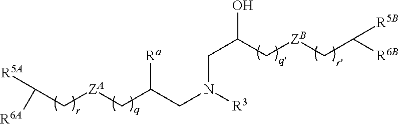

wherein:
q and q′ are each independently integers from 0 to 12;
r and r′ are each independently integers from 0 to 6, wherein at least one of r or r′ is not 0;
Z^{A} and Z^{B} are each independently selected from \hat{ }—C(O)O—, \hat{ }—OC(O), and —OC(O)O—; where \hat{ } denotes the attachment point to —(CH₂)_{q}— or —(CH₂)_{q′}—; and
R^{5A}, R^{5B}, R^{6A}, and R^{6B} are each independently C₁-C₂₀ alkyl or C₂-C₂₀ alkenyl.

In some embodiments of Formula (23), Z^{A} and Z^{B} are \hat{ }—C(O)O—, and the ionizable lipid is of Formula (23a-1):

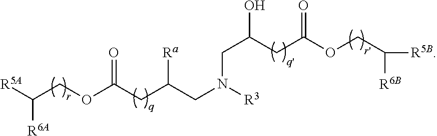

In some embodiments of Formula (23), Z^{A} and Z^{B} are \hat{ }—OC(O)—, and the ionizable lipid is of Formula (23a-2)

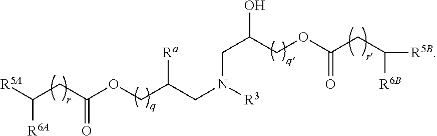

In some embodiments of Formula (23), Z^{A} and Z^{B} are —O(C)(O)O—, and the ionizable lipid is represented by Formula (23a-3):

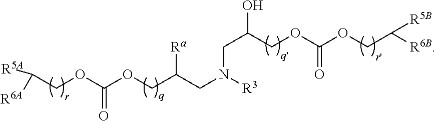

In some embodiments of Formula (22), R² is C₁-C₃₀ alkyl, and the ionizable lipid is of Formula (25):

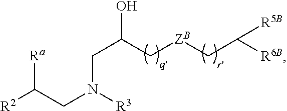

or is a pharmaceutically acceptable salt thereof, wherein:
Z^{B} is selected from \hat{ }—C(O)O—, \hat{ }—OC(O)—, and —OC(O)O—; where \hat{ } denotes the attachment point to —(CH₂)_{q′}—;
R^{5B} and R^{6B} are each independently C₁-C₂₀ alkyl or C₂-C₂₀ alkenyl;
q′ is an integer from 0 to 12; and
r′ is an integer from 1 to 6.

In some embodiments of Formula (25), Z^{B} is \hat{ }—C(O)O—, and the ionizable lipid is of Formula (25a-1):

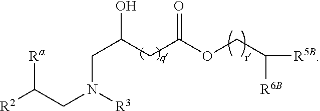

In some embodiments of Formula (25), Z^{B} is \hat{ }—OC(O)—, and the ionizable lipid is of Formula (25a-2):

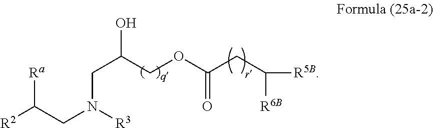

In some embodiments of Formula (25), Z^{B} is —OC(O)O—, and the ionizable lipid is of Formula (25a-3):

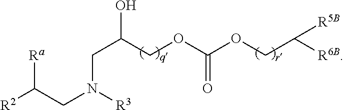

In some embodiments, an ionizable lipid is selected from the table below:

| Lipid | Structure |
|---|---|
| 100-a | 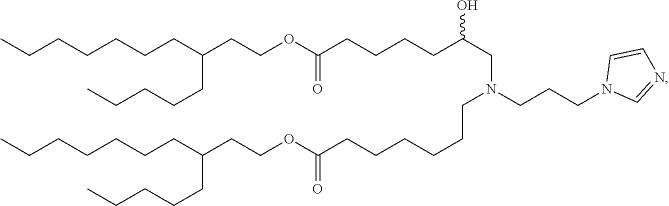 [SMILES: CCCCCCCC(CCCCC)CCOC(=O)CCCCCCN(CCCn1ccnc1)CC(O)CCCCC(=O)OCCC(CCCCC)CCCCCCC] |
| 100-b | 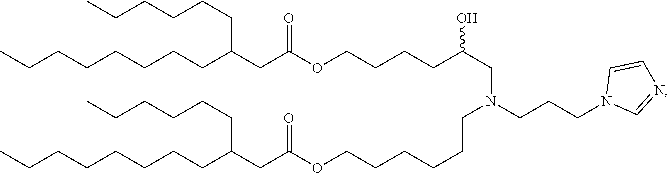 [SMILES: CCCCCCCCC(CCCCCC)CC(=O)OCCCCCCN(CCCn1ccnc1)CC(O)CCCCOC(=O)CC(CCCCCC)CCCCCCCC] |
| 100-c | 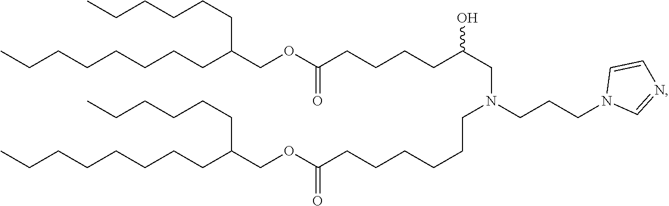 [SMILES: CCCCCCCCC(CCCCCC)COC(=O)CCCCCCN(CCCn1ccnc1)CC(O)CCCCC(=O)OCC(CCCCCC)CCCCCCCC] |
| 100-d | 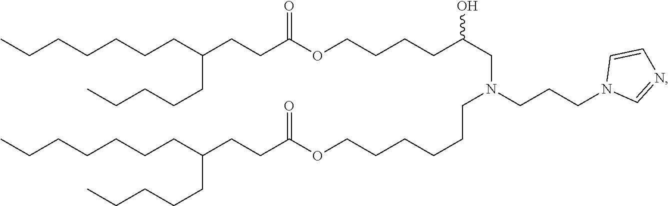 [SMILES: CCCCCCCC(CCCCC)CCC(=O)OCCCCCCN(CCCn1ccnc1)CC(O)CCCCOC(=O)CCC(CCCCC)CCCCCCC] |
| 100-e | 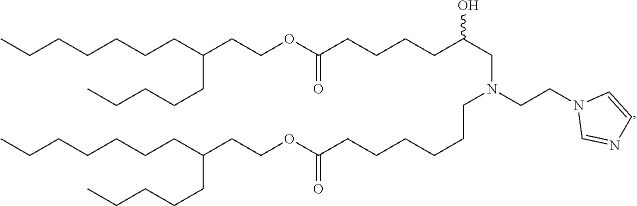 [SMILES: CCCCCCCC(CCCCC)CCOC(=O)CCCCCCN(CCn1ccnc1)CC(O)CCCCC(=O)OCCC(CCCCC)CCCCCCC] |
| | and |
| 100-f | 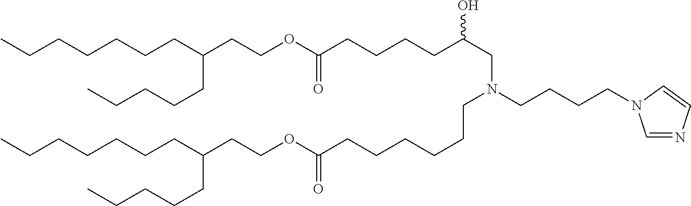 [SMILES: CCCCCCCC(CCCCC)CCOC(=O)CCCCCCN(CCCCn1ccnc1)CC(O)CCCCC(=O)OCCC(CCCCC)CCCCCCC] |

In some embodiments, an ionizable lipid is selected from the table below:

| Lipid | Structure |
|---|---|
| 1-al | 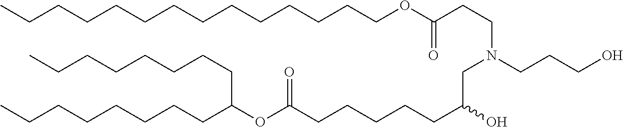 [SMILES: CCCCCCCCCCCCCCOC(=O)CCN(CCCO)CC(O)CCCCCC(=O)OC(CCCCCCCC)CCCCCCCC] |
| 1-am | 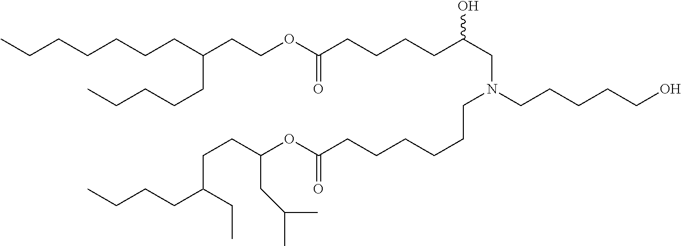 [SMILES: CCCCCCCC(CCCCC)CCOC(=O)CCCCC(O)CN(CCCCCO)CCCCCCC(=O)OC(CCC(CC)CCCC)CC(C)C] |
| 1-an | 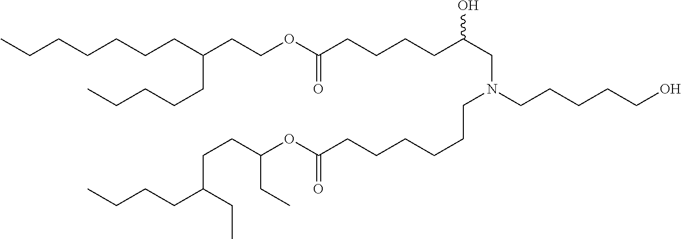 [SMILES: CCCCCCCC(CCCCC)CCOC(=O)CCCCC(O)CN(CCCCCO)CCCCCCC(=O)OC(CC)CCC(CC)CCCC] |
| 1-ao | 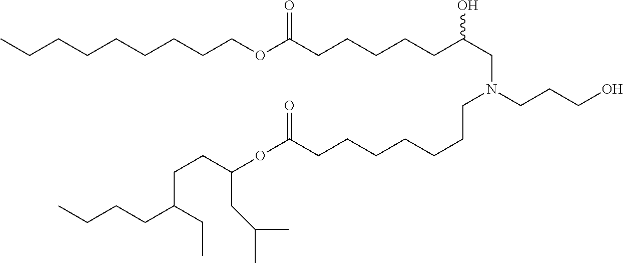 [SMILES: CCCCCCCCCOC(=O)CCCCCC(O)CN(CCCO)CCCCCCCC(=O)OC(CCC(CC)CCCC)CC(C)C] |
| 1-ap | 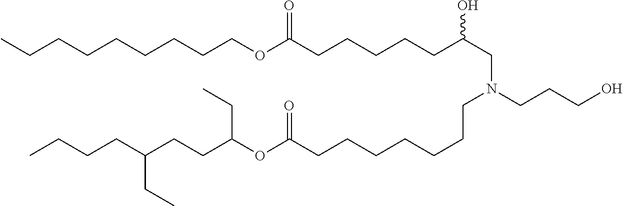 [SMILES: CCCCCCCCCOC(=O)CCCCCC(O)CN(CCCO)CCCCCCCC(=O)OC(CC)CCC(CC)CCCC] |
| 1-aq | 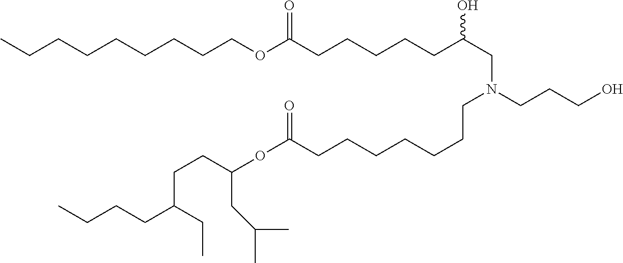 [SMILES: CCCCCCCCCOC(=O)CCCCCC(O)CN(CCCO)CCCCCCCC(=O)OC(CCC(CC)CCCC)CC(C)C] |
| 1-ar | 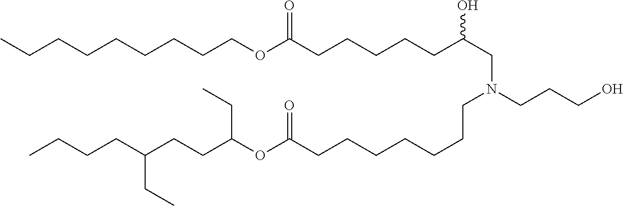 [SMILES: CCCCCCCCCOC(=O)CCCCCC(O)CN(CCCO)CCCCCCCC(=O)OC(CC)CCC(CC)CCCC] |
| 1-as | 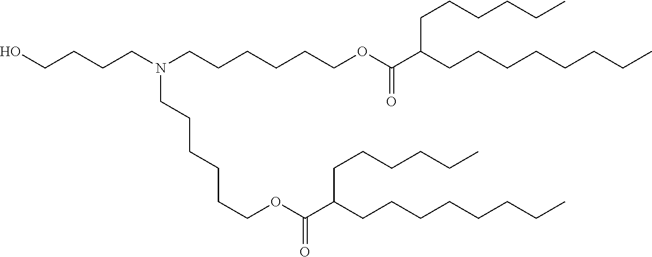 [SMILES: CCCCCCCCC(CCCCCC)C(=O)OCCCCCCN(CCCCO)CCCCCCOC(=O)C(CCCCCC)CCCCCCCC] |
| 1-at | 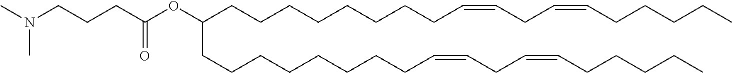 [SMILES: CCCCC/C=C\C/C=C\CCCCCCCCC(CCCCCCCC/C=C\C/C=C\CCCCC)OC(=O)CCCN(C)C] |
| 1-au | 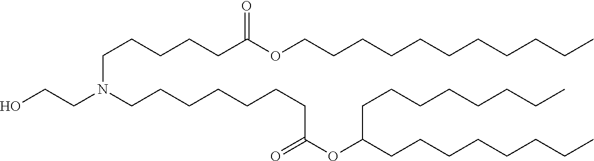 [SMILES: CCCCCCCCCCCOC(=O)CCCCCN(CCO)CCCCCCCC(=O)OC(CCCCCCCC)CCCCCCCC] |

In some embodiments, an ionizable lipid is described in US patent publication number 20190321489. In some embodiments, an ionizable lipid is described in international patent publication WO 2010/053572, incorporated herein by reference. In some embodiments, an ionizable lipid is C12-200, described at paragraph of WO 2010/053572.

Several ionizable lipids have been described in the literature, many of which are commercially available. In certain embodiments, such ionizable lipids are included in the transfer vehicles described herein. Other suitable cationic lipids include, for example, ionizable cationic lipids as described in U.S. provisional patent application 61/617,468, filed Mar. 29, 2012 (incorporated herein by reference), such as, e.g., (15Z,18Z)-N,N-dimethyl-6-(9Z,12Z)-octadeca-9,12-dien-1-yl)tetracosa-15,18-dien-1-amine (HGT5000), (15Z,18Z)-N,N-dimethyl-6-((9Z,12Z)-octadeca-9,12-dien-1-yl)tetracosa-4,15,18-trien-1-amine (HGT5001), and (15Z,18Z)-N,N-dimethyl-6-((9Z,12Z)-octadeca-9,12-dien-1-yl)tetracosa-5,15, 18-trien-1-amine (HGT5002), C12-200 (described in WO 2010/053572), 2-(2,2-di((9Z,12Z)-octadeca-9,12-dien-1-yl)-1,3-dioxolan-4-yl)-N,N-dimethylethanamine (DLinKC2-DMA)) (See, WO 2010/042877; Semple et al., Nature Biotech. 28:172-176 (2010)), 2-(2,2-di((9Z,2Z)-octadeca-9,12-dien-1-yl)-1,3-dioxolan-4-yl)-N,N-dimethylethanamine (DLin-KC2-DMA), (3S,10R,13R,17R)-10,13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate (ICE), (15Z,18Z)-N,N-dimethyl-6-(9Z,12Z)-octadeca-9,12-dien-1-yl)tetracosa-15,18-dien-1-amine (HGT5000), (15Z,18Z)-N,N-dimethyl-6-((9Z,12Z)-octadeca-9,12-dien-1-yl)tetracosa-4,15,18-trien-1-amine (HGT5001), (15Z,18 Z)-N,N-dimethyl-6-((9Z,12Z)-octadeca-9,12-dien-1-yl)tetracosa-5,15,18-trien-1-amine (HGT5002), 5-carboxyspermylglycine-dioctadecylamide (DOGS), 2,3-dioleyloxy-N-[2(spermine-carboxamido)ethyl]-N,N-dimethyl-1-propanaminium (DOSPA) (Behr et al. Proc. Nat.'l Acad. Sci. 86, 6982 (1989); U.S. Pat. Nos. 5,171,678; 5,334,761), 1,2-Dioleoyl-3-Dimethylammonium-Propane (DODAP), 1,2-Dioleoyl-3-Trimethylammonium-Propane or (DOTAP). Contemplated ionizable lipids also include 1,2-distcaryloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), N-dioleyl-N,N-dimethylammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylamninopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA) or GL67, or mixtures thereof.

Also contemplated are cationic lipids such as dialkylamino-based, imidazole-based, and guanidinium-based lipids. For example, also contemplated is the use of the ionizable lipid (3S,10R, 13R, 17R)-10,13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate (ICE), as disclosed in International Application No. PCT/US2010/058457, incorporated herein by reference.

Also contemplated are ionizable lipids such as the dialkylamino-based, imidazole-based, and guanidinium-based lipids.

In some embodiments, an ionizable lipid is described by US patent publication number 20190314284.

The ionizable lipids include those disclosed in international patent application PCT/US2019/025246, and US patent publications 2017/0190661 and 2017/0114010, incorporated herein by reference in their entirety.

In some embodiments, an ionizable lipid is as described in international patent application PCT/US2019/015913.

Preparation methods for the above compounds and compositions are described herein below and/or known in the art.

It will be appreciated by those skilled in the art that in the process described herein the functional groups of intermediate compounds may need to be protected by suitable protecting groups. Such functional groups include, e.g., hydroxyl, amino, mercapto, and carboxylic acid. Suitable protecting groups for hydroxyl include, e.g., trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino, and guanidino include, e.g., t-butoxycarbonyl, benzyloxycarbonyl, and the like. Suitable protecting groups for mercapto include, e.g., —C(O)—R″ (where R″ is alkyl, aryl, or arylalkyl), p-methoxybenzyl, trityl, and the like. Suitable protecting groups for carboxylic acid include, e.g., alkyl, aryl, or arylalkyl esters. Protecting groups may be added or removed in accordance with standard techniques, which are known to one skilled in the art and as described herein. The use of protecting groups is described in detail in, e.g., Green, T. W. and P. G. M. Wutz, Protective Groups in Organic Synthesis (1999), 3rd Ed., Wiley. As one of skill in the artwould appreciate, the protecting group may also be a polymer resin such as a Wang resin, Rink resin, or a 2-chlorotrityl-chloride resin.

It will also be appreciated by those skilled in the art, although such protected derivatives of compounds described herein may not possess pharmacological activity as such, they may be administered to a mammal and thereafter metabolized in the body to form compounds described herein which are pharmacologically active. Such derivatives may therefore be described as prodrugs. All prodrugs of compounds described herein are included within the scope of the present disclosure.

Furthermore, all compounds described herein which exist in free base or acid form can be converted to their pharmaceutically acceptable salts by treatment with the appropriate inorganic or organic base or acid by methods known to one skilled in the art. Salts of the compounds described herein can also be converted to their free base or acid form by standard techniques.

Preparation methods for the above compounds and compositions are described herein below and/or known in the art. It is understood that one skilled in the art may be able to make these compounds by similar methods or by combining other methods known to one skilled in the art.

a. Amine Lipids

In certain embodiments, transfer vehicle compositions for the delivery of circular RNA comprise an amine lipid. In certain embodiments, an ionizable lipid is an amine lipid.

In some embodiments, an amine lipid is described in international patent application PCT/US2018/053569. In some embodiments, the amine lipid is Lipid E of WO 2022/261490 and WO 2023/056033, which is (9Z, 12Z)-3-((4,4-bis(octyloxy)butanoyl)oxy)-2-((((3-(diethylamino)propoxy)carbonyl)oxy)methyl)propyl octadeca-9, 12-dienoate. Lipid E may be synthesized according to WO2015/095340 (e.g., pp. 84-86). In certain embodiments, the amine lipid is an equivalent to Lipid E. In certain embodiments, an amine lipid is an analog of Lipid E.

Amine lipids and other biodegradable lipids suitable for use in the transfer vehicles, e.g., lipid nanoparticles, described herein are biodegradable in vivo. The amine lipids described herein have low toxicity (e.g., are tolerated in animal models without adverse effect in amounts of greater than or equal to 10 mg/kg). In certain embodiments, transfer vehicles composing an amine lipid include those where at least 75% of the amine lipid is cleared from the plasma within 8, 10, 12, 24, or 48 hours, or 3, 4, 5, 6, 7, or 10 days.

Biodegradable lipids include, for example, the biodegradable lipids of WO2017/173054, WO2015/095340, and WO2014/136086.

Lipid clearance may be measured by methods known by persons of skill in the art. See, for example, Maier, M. A., et al. Biodegradable Lipids Enabling Rapidly Eliminated Lipid Nanoparticles for Systemic Delivery of RNAi Therapeutics. Mol. Ther. 2013, 21(8), 1570-78.

Transfer vehicle compositions comprising an amine lipid can lead to an increased clearance rate. In some embodiments, the clearance rate is a lipid clearance rate, for example the rate at which a lipid is cleared from the blood, serum, or plasma. In some embodiments, the clearance rate is an RNA clearance rate, for example the rate at which a circRNA is cleared from the blood, serum, or plasma. In some embodiments, the clearance rate is the rate at which transfer vehicles are cleared from the blood, serum, or plasma. In some embodiments, the clearance rate is the rate at which transfer vehicles are cleared from a tissue, such as liver tissue or spleen tissue. In certain embodiments, a high rate of clearance leads to a safety profile with no substantial adverse effects. The amine lipids and biodegradable lipids may reduce transfer vehicle accumulation in circulation and in tissues. In some embodiments, a reduction in transfer vehicle accumulation in circulation and in tissues leads to a safety profile with no substantial adverse effects.

Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood, where pH is approximately 7.35, the lipid, such as an amine lipid, may not be protonated and thus bear no charge.

The ability of a lipid to bear a charge is related to its intrinsic pKa. In some embodiments, the amine lipids of the present disclosure may each, independently, have a pKa in the range of from about 5.1 to about 7.4. In some embodiments, the bioavailable lipids of the present disclosure may each, independently, have a pKa in the range of from about 5.1 to about 7.4. For example, the amine lipids of the present disclosure may each, independently, have a pKa in the range of from about 5.8 to about 6.5. Lipids with a pKa ranging from about 5.1 to about 7.4 are effective for delivery of cargo in vivo, e.g., to the liver. Further, it has been found that lipids with a pKa ranging from about 5.3 to about 6.4 are effective for delivery in vivo, e.g., into tumors. See, e.g., WO2014/136086.

b. Lipids Containing a Disulfide Bond

In some embodiments, the ionizable lipid is described in U.S. Pat. No. 9,708,628.

In some embodiments, the lipid may have an —S—S— (disulfide) bond. The production method for such a compound includes, for example, a method including producing

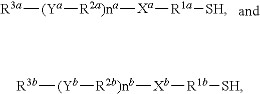

and
subjecting them to oxidation (coupling) to give a compound containing —S—S—, a method including sequentially bonding necessary parts to a compound containing an —S—S— bond to finally obtain the compound and the like. Preferred is the latter method.

An example of the latter method is described on pages 470-472 of WO 2022/261490, which is incorporated by reference herein in its entirety.

Exemplary lipids containing a disulfide bond are described in WO 2022/261490, including the lipid represented by structure (XXII) described therein at pages 459-469 and structures 1-15 of Table 15b, which are incorporated by reference herein in its entirety. WO 2023/056033 is also incorporated by reference herein in its entirety.

c. Further Exemplary Lipids or Lipid-Like Compounds

In some embodiments, an ionizable lipid is described in U.S. Pat. No. 9,765,022.

In some embodiments, a lipid-like compound is represented by structure (XXIII), and is described in WO 2022/261490 and WO 2023/056033, including the lipid compounds comprising the exemplary hydrophilic heads, hydrophobic tails, linkers, and exemplary lipid-like compounds described therein.

As described therein, the lipid-like compounds of structure XXIII of WO 2022/261490 and WO 2023/056033 and other lipid-like compounds can be prepared by methods well known the art. See Wang et al., ACS Synthetic Biology, 1, 403-07 (2012); Manoharan, et al., International Patent Application Publication WO 2008/042973; and Zugates et al., U.S. Pat. No. 8,071,082. WO 2022/261490 and WO 2023/056033 describe an exemplary route of synthesis of the lipid-like compounds, and other suitable starting materials and routes of synthesis known in the art. See, for example, R. Larock, Comprehensive Organic Transformations (2nd Ed., VCH Publishers 1999); P. G. M. Wuts and T. W. Greene, Greene's Protective Groups in Organic Synthesis (4th Ed., John Wiley and Sons 2007); L. Fieser and M. Fieser, Fieser and Fieser's Reagents for Organic Synthesis (John Wiley and Sons 1994); and L. Paquette, ed., Encyclopedia of Reagents for Organic Synthesis (2nd ed., John Wiley and Sons 2009) and subsequent editions thereof. Certain lipid-like compounds may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

As mentioned above, these lipid-like compounds are useful for delivery of pharmaceutical agents. They can be preliminarily screened for their efficacy in delivering pharmaceutical agents by an in vitro assay and then confirmed by animal experiments and clinic trials. Other methods will also be apparent to those of ordinary skill in the art.

The above described complexes can be prepared using procedures described in publications such as Wang et al., ACS Synthetic Biology, 1, 403-07 (2012). Generally, they are obtained by incubating a lipid-like compound and a pharmaceutical agent in a buffer such as a sodium acetate buffer or a phosphate buffered saline (“PBS”).

d. Hydrophilic Groups

In certain embodiments, the selected hydrophilic functional group or moiety may alter or otherwise impart properties to the compound or to the transfer vehicle of which such compound is a component (e.g., by improving the transfection efficiencies of a lipid nanoparticle of which the compound is a component). For example, the incorporation of guanidinium as a hydrophilic head-group in the compounds disclosed herein may promote the fusogenicity of such compounds (or of the transfer vehicle of which such compounds are a component) with the cell membrane of one or more target cells, thereby enhancing, for example, the transfection efficiencies of such compounds. It has been hypothesized that the nitrogen from the hydrophilic guanidinium moiety forms a six-membered ring transition state which grants stability to the interaction and thus allows for cellular uptake of encapsulated materials. (Wender, et al., Adv. Drug Del. Rev. (2008) 60: 452-472.) Similarly, the incorporation of one or more amino groups or moieties into the disclosed compounds (e.g., as a head-group) may further promote disruption of the endosomal/lysosomal membrane of the target cell by exploiting the fusogenicity of such amino groups. This is based not only on the pKa of the amino group of the composition, but also on the ability of the amino group to undergo a hexagonal phase transition and fuse with the target cell surface, i.e. the vesicle membrane. (Koltover, et al. Science (1998) 281: 78-81.) The result is believed to promote the disruption of the vesicle membrane and release of the lipid nanoparticle contents into the target cell.

Similarly, in certain embodiments the incorporation of, for example, imidazole as a hydrophilic head-group in the compounds disclosed herein may serve to promote endosomal or lysosomal release of, for example, contents that are encapsulated in a transfer vehicle (e.g., lipid nanoparticle) of the present disclosure. Such enhanced release may be achieved by one or both of a proton-sponge mediated disruption mechanism and/or an enhanced fusogenicity mechanism. The proton-sponge mechanism is based on the ability of a compound, and in particular a functional moiety or group of the compound, to buffer the acidification of the endosome. This may be manipulated or otherwise controlled by the pKa of the compound or of one or more of the functional groups comprising such compound (e.g., imidazole). Accordingly, in certain embodiments the fusogenicity of, for example, the imidazole-based compounds disclosed herein (e.g., HGT4001 and HGT4004) are related to the endosomal disruption properties, which are facilitated by such imidazole groups, which have a lower pKa relative to other traditional ionizable lipids. Such endosomal disruption properties in turn promote osmotic swelling and the disruption of the liposomal membrane, followed by the transfection or intracellular release of the polynucleotide materials loaded or encapsulated therein into the target cell. This phenomenon can be applicable to a variety of compounds with desirable pKa profiles in addition to an imidazole moiety. Such embodiments also include multi-nitrogen based functionalities such as polyamines, poly-peptide (histidine), and nitrogen-based dendritic structures.

Exemplary ionizable and/or cationic lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety. International patent application WO 2019/131770 is also incorporated herein by reference in its entirety.

### B. Stabilizing Lipids (e.g., PEG Lipids)

A stabilizing lipid or surface stabilizing lipid may be used to enhance the structure of the LNP. A stabilizing lipid as contemplated herein may be a polyethylene glycol (PEG)-modified phospholipid.

The use and inclusion of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000](C8 PEG-2000 ceramide) in the liposomal and pharmaceutical compositions described herein is contemplated, preferably in combination with one or more of the compounds and lipids disclosed herein. Contemplated PEG-modified lipids include, but are not limited to, a polyethylene glycol chain of up to 5 kDa in length covalently attached to a lipid with alkyl chain(s) of C6-C20 length. In some embodiments, the PEG-modified lipid employed in the compositions and methods described herein is 1,2-dimyristoyl-sn-glycerol, methoxypolyethylene Glycol (2000 MW PEG) “DMG-PEG2000.” The addition of PEG-modified lipids to the lipid delivery vehicle may prevent complex aggregation and may also provide a means for increasing circulation lifetime and increasing the delivery of the lipid-polynucleotide composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivatized lipids may comprise a molar ratio from about 0% to about 20%, about 0.5% to about 20%, about 1% to about 15%, about 4% to about 10%, or about 2% of the total lipid present in a liposomal lipid nanoparticle.

In some embodiments, the lipid moiety of the PEG-lipids includes those having lengths of from about C14 to about C22, such as from about C14 to about C16. In some embodiments, a PEG moiety, for example a mPEG-NH2, has a size of about 1000, about 2000, about 5000, about 10,000, about 15,000 or about 20,000 daltons

In an embodiment, a PEG-modified lipid is described in International Pat. Appl. No. PCT/US2019/015913, which is incorporated herein by reference in their entirety. In an embodiment, a transfer vehicle comprises one or more PEG-modified lipids.

Non-limiting examples of PEG-modified lipids include PEG-modified phosphatidylethanolamines and phosphatidic acids, PEG-ceramide conjugates (e.g., PEG-CerC14 or PEG-CerC20), PEG-modified dialkylamines and PEG-modified 1,2-diacyloxypropan-3-amines, PEG-modified diacylglycerols, PEG-modified dialkylglycerols, and mixtures thereof. In some further embodiments, a PEG-modified lipid may be, e.g., PEG-c-DOMG, PEG-DMG, PEG-DLPE, PEG-DMPE, PEG-DPPC, PEG-DSPE, PEG-DAG, PEG-S-DAG, PEG-PE, PEG-S-DMG, PEG-CER, PEG-dialkoxypropylcarbamate, PEG-OR, PEG-OH, PEG-c-DOMG, or PEG-1.

In some still further embodiments, the PEG-modified lipid includes, but is not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the lipid nanoparticles described herein can comprise a lipid modified with a non-diffusible PEG. Non-limiting examples of non-diffusible PEGs include PEG-DSG and PEG-DSPE. In one embodiment, the lipid nanoparticles herein comprise PEG-DSPC.

In some embodiments the PEG-modified lipids are a modified form of PEG-DMG. PEG-DMG has the following structure:

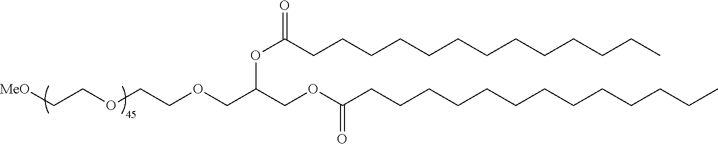

In some embodiments, the PEG lipid is a compound of Formula (P1):

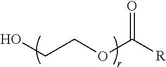

or a salt or isomer thereof, wherein:
r is an integer between 1 and 100;
R is C10-40 alkyl, C10-40 alkenyl, or C10-40 alkynyl; and optionally one or more methylene groups of R are independently replaced with C3-10 carbocyclylene, 4 to 10 membered heterocyclylene, C6-10 arylene, 4 to 10 membered heteroarylene, —N(RN)—, —O—, —S—, —C(O)—, —C(O)N(RN)—, —NRNC(O)—, —NRNC(O)N(RN)—, —C(O)O—, —OC(O)—, —OC(O)O—, —OC(O)N(RN)—, —NRNC(O)O—, —C(O)S—, —SC(O)—, —C(═NRN)—, —C(═NRN)N(RN)—, —NRNC(═NRN)—, —NRNC(═NRN)N(RN)—, —C(S)—, —C(S)N(RN)—, —NRNC(S)—, —NRNC(S)N(RN)—, —S(O)—, —OS(O)—, —S(O)O—, —OS(O)O—, —OS(O)2-, —S(O)2O-, —OS(O)2O-, —N(RN)S(O)—, —S(O)N(RN)—, —N(RN)S(O)N(RN)—, —OS(O)N(RN)—, —N(RN)S(O)O—, —S(O)2-, —N(RN)S(O)2-, —S(O)2N(RN)—, —N(RN)S(O)2N(RN)—, —OS(O)2N(RN)—, or —N(RN)S(O)2O-; and
each instance of RN is independently hydrogen, C1-6 alkyl, or a nitrogen protecting group.

For example, R is C17 alkyl. For example, the PEG lipid is a compound of Formula (P1-a):

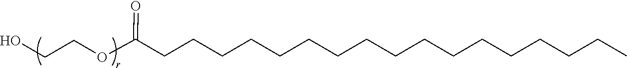

or a salt or isomer thereof, wherein r is an integer between 1 and 100.

In some embodiments the PEG-modified lipids are a modified form of PEG-C18, or PEG-1. PEG-1 has the following structure:

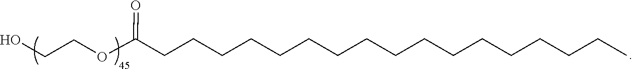

PEG-lipids are known in the art, such as those described in U.S. Pat. No. 8,158,601 and International Pat. Publ. No. WO2015/130584 A2, which are incorporated herein by reference in their entirety. In one embodiment, PEG lipids can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

### C. Helper Lipids

In some embodiments, the transfer vehicle (e.g., LNP) described herein comprises one or more non-cationic helper lipids. In some embodiments, the helper lipid is a phospholipid. In some embodiments, the helper lipid is a phospholipid substitute or replacement. In some embodiments, the phospholipid or phospholipid substitute can be, for example, one or more saturated or (poly)unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the helper lipid is a 1,2-distearoyl-177-glycero-3-phosphocholine (DSPC) analog, a DSPC substitute, oleic acid, or an oleic acid analog.

In some embodiments, a helper lipid is a non-phosphatidyl choline (PC) zwitterionic lipid, a DSPC analog, oleic acid, an oleic acid analog, or a DSPC substitute.

In some embodiments, a helper lipid is described in PCT/US2018/053569. Helper lipids suitable for use in a lipid composition of the disclosure include, for example, a variety of neutral, uncharged or zwitterionic lipids. Such helper lipids are preferably used in combination with one or more of the compounds and lipids disclosed herein. Examples of helper lipids include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoylsn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-paimitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), paimitoyioieoyl phosphatidylcholine (POPC), lysophosphatidyl choline, dioleoyl phosphatidylethanol amine (DOPE) dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the helper lipid may be distearoylphosphatidylcholine (DSPC) or dimyristoyl phosphatidyl ethanolamine (DMPE). In another embodiment, the helper lipid may be distearoylphosphatidylcholine (DSPC). Helper lipids function to stabilize and improve processing of the transfer vehicles. Such helper lipids are preferably used in combination with other excipients, for example, one or more of the ionizable lipids disclosed herein. In some embodiments, when used in combination with an ionizable lipid, the helper lipid may comprise a molar ratio of 5% to about 90%, or about 10% to about 70% of the total lipid present in the lipid nanoparticle.

### D. Structural Lipids

The transfer vehicles described herein comprise one or more structural lipids. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid is an analog of cholesterol. In certain embodiments, the structural lipid includes cholesterol and a corticosteroid (such as, for example, prednisolone, dexamethasone, prednisone, and hydrocortisone), or a combination thereof.

In an embodiment, a structural lipid is described in international patent application PCT/US2019/015913.

In some embodiments, the structural lipid is a sterol (e.g., phytosterols or zoosterols). In certain embodiments, the structural lipid is a steroid. For example, sterols can include, but are not limited to, cholesterol, β-sitosterol, fecosterol, ergosterol, sitosterol, campesterol, stigmasterol, brassicasterol, ergosterol, tomatidine, tomatine, ursolic acid, or alpha-tocopherol. In certain embodiments, the structural lipid is cholesterol. In certain embodiments, the structural lipid is an analog of cholesterol. In certain embodiments, the structural lipid is alpha-tocopherol.

The transfer vehicles described herein comprise one or more structural lipids. Incorporation of structural lipids in a transfer vehicle, e.g., a lipid nanoparticle, may help mitigate aggregation of other lipids in the particle. In certain embodiments, the structural lipid includes cholesterol and a corticosteroid (such as, for example, prednisolone, dexamethasone, prednisone, and hydrocortisone), or a combination thereof.

In some embodiments, a transfer vehicle includes an effective amount of an immune cell delivery potentiating lipid, e.g., a cholesterol analog or an amino lipid or combination thereof, that, when present in a transfer vehicle, e.g., an lipid nanoparticle, may function by enhancing cellular association and/or uptake, internalization, intracellular trafficking and/or processing, and/or endosomal escape and/or may enhance recognition by and/or binding to immune cells, relative to a transfer vehicle lacking the immune cell delivery potentiating lipid. Accordingly, while not intending to be bound by any particular mechanism or theory, in one embodiment, a structural lipid or other immune cell delivery potentiating lipid of the disclosure binds to C1q or promotes the binding of a transfer vehicle comprising such lipid to C1q. Thus, for in vitro use of the transfer vehicles of the disclosure for delivery of a nucleic acid molecule to an immune cell, culture conditions that include C1q are used (e.g., use of culture media that includes serum or addition of exogenous C1q to serum-free media). For in vivo use of the transfer vehicles of the disclosure, the requirement for C1q is supplied by endogenous C1q.

In certain embodiments, the structural lipid is cholesterol. In certain embodiments, the structural lipid is an analog of cholesterol.

In some embodiments, a structural lipid of any one of the disclosed embodiments is selected from the structures set forth in Table 16 of WO 2022/261490 and Table 16 of WO 2023/056033, which are incorporated by reference herein in their entireties.

### E. Lipid Nanoparticle (LNP) Formulations

In certain embodiments, the transfer vehicle comprises a lipid. In certain embodiments, the transfer vehicle comprises an ionizable lipid. In certain embodiments, the transfer vehicle comprises an ionizable lipid in combination with other lipids, e.g., a structural lipid, and/or a PEG-modified lipid.

In certain embodiments, the transfer vehicle is a lipid nanoparticle (LNP), which may be capable of delivering the one or more circular RNA constructs to one or more target cells. In certain embodiments the transfer vehicle is capable of delivering the circular RNA construct to a human immune cell present in a human subject, such that the expression sequence encoding a binding molecule (e.g., CAR) is translated in the human immune cell and expressed on the surface of the human immune cell.

In certain embodiments, the transfer vehicles are prepared to encapsulate one or more materials or therapeutic agents (e.g., circular RNA). The process of incorporating a desired therapeutic agent (e.g., circular RNA) into a transfer vehicle is referred to herein as or “loading” or “encapsulating” (Lasic, et al., FEBS Lett., 312: 255-258, 1992). The transfer vehicle-loaded or -encapsulated materials (e.g., circular RNA) may be completely or partially located in the interior space of the transfer vehicle, within a bilayer membrane of the transfer vehicle, or associated with the exterior surface of the transfer vehicle.

In some embodiments, a transfer vehicle encapsulates circular RNA. In some embodiments, the transfer vehicle encapsulates at least one circular RNA construct and comprises an ionizable lipid. In some embodiments, the transfer vehicle encapsulates at least one circular RNA construct and comprises an ionizable lipid and an additional lipid selected from a structural lipid, a helper lipid, and a PEG-modified lipid. In some embodiments, the transfer vehicle encapsulates at least one circular RNA construct and comprises an ionizable lipid, a structural lipid, a helper lipid, and/or a PEG-modified lipid. In some embodiments, a transfer vehicle encapsulates at least one circular RNA construct and comprises an ionizable lipid, a structural lipid, a PEG-modified lipid, and a helper lipid. In some embodiments, the transfer vehicle is a lipid nanoparticle.

Without wishing to be bound by theory, it is thought that transfer vehicles described herein shield encapsulated circular RNA from degradation and provide for effective delivery of circular RNA to target cells in vivo and in vitro.

In certain embodiments, the transfer vehicles are formulated based in part upon their ability to facilitate the transfection (e.g., of a circular RNA) of a target cell. In another embodiment, the transfer vehicles may be selected and/or prepared to optimize delivery of circular RNA to a target cell, tissue or organ. For example, if the target cell is a hepatocyte, the properties of the compositions (e.g., size, charge and/or pH) may be optimized to effectively deliver such composition (e.g., lipid nanoparticles) to the target cell or organ, reduce immune clearance and/or promote retention in the target cell or organ. Alternatively, if the target tissue is the central nervous system, the selection and preparation of the transfer vehicle must consider penetration of, and retention within. the blood brain barrier and/or the use of alternate means of directly delivering such compositions to such target tissue (e.g., via intracerebrovascular administration). In certain embodiments, the transfer vehicles may be combined with agents that facilitate the transfer of encapsulated materials across the blood brain barrier (e.g., agents which disrupt or improve the permeability of the blood brain barrier and thereby enhance the transfer of circular RNA to the target cells). While the transfer vehicles described herein can facilitate introduction of circular RNA into target cells, the addition of polycations (e.g., poly L-lysine and protamine) as a copolymer to one or more of the lipid nanoparticles that comprise the pharmaceutical compositions can in some instances markedly enhance the transfection efficiency of several types of transfer vehicles by 2-28 fold in a number of cell lines both in vitro and in vivo (See, N. J. Caplen, et al., Gene Ther. 1995; 2: 603; S. Li, et al., Gene Ther. 1997; 4, 891.).

Transfer vehicles described herein can allow the encapsulated polynucleotide to reach the target cell or may preferentially allow the encapsulated polynucleotide to reach the target cells or organs on a discriminatory basis. Alternatively, the transfer vehicles may limit the delivery of encapsulated polynucleotides to other non-targeted cells or organs where the presence of the encapsulated polynucleotides may be undesirable or of limited utility.

Loading or encapsulating a polynucleotide, e.g., circular RNA, into a transfer vehicle may serve to protect the polynucleotide from an environment (e.g., serum) which may contain enzymes or chemicals that degrade such polynucleotides and/or systems or receptors that cause the rapid excretion of such polynucleotides. Accordingly, in some embodiments, the compositions described herein are capable of enhancing the stability of the encapsulated polynucleotide(s), particularly with respect to the environments into which such polynucleotides will be exposed.

In certain embodiments, the transfer vehicles described herein are prepared by combining multiple lipid components (e.g., one or more of the compounds disclosed herein) with one or more polymer components.

A lipid nanoparticle may be comprised of additional lipid combinations in various ratios. The selection of ionizable lipids, helper lipids, structural lipids, and/or PEG-modified lipids that make up the lipid nanoparticles, as well as the relative molar ratio of such lipids to each other, is based upon the characteristics of the selected lipid(s), the nature of the intended target cells or tissues and the characteristics of the materials or polynucleotides to be delivered by the lipid nanoparticle. Additional considerations include, for example, the saturation of the alkyl chain, as well as the size, charge, pH, pKa, fusogenicity and toxicity of the selected lipid(s).

The formation of a lipid nanoparticle (LNP) described herein may be accomplished by any methods known in the art. See, e.g., U.S. Pat. Pub. No. US2012/0178702 A1, which is incorporated herein by reference in its entirety. Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51:8529-8533; and Maier et al. (2013) Molecular Therapy 21, 1570-1578 (the contents of each of which are incorporated herein by reference in their entirety). Lipid nanoparticles, formulations, and methods of preparation are described in, e.g., International Pat. Pub. No. WO 2011/127255 or WO 2008/103276, U.S. Pat. Pub. No. US2005/0222064 A1, U.S. Pat. Pub. No. US2013/0156845 A1, International Pat. Pub. No. WO2013/093648 A2, WO2012/024526 A2, U.S. Pat. Pub. No. US2013/0164400 A1, and U.S. Pat. No. 8,492,359, all of which are incorporated herein by reference in their entirety.

In some embodiments, the lipid nanoparticle comprises one or more cationic lipids, ionizable lipids, or poly β-amino esters. In some embodiments, the nanoparticle comprises one or more non-cationic lipids. In some embodiments, the lipid nanoparticle comprises one or more PEG-modified lipids, polyglutamic acid lipids, or hyaluronic acid lipids. In some embodiments, the lipid nanoparticle comprises cholesterol. In some embodiments, the lipid nanoparticle comprises arachidonic acid, leukotriene, or oleic acid. In some embodiments, the lipid nanoparticle comprises a targeting moiety, wherein the targeting moiety mediates receptor-mediated endocytosis selectively into cells of a selected cell population in the absence of cell selection or purification. In some embodiments, the lipid nanoparticle comprises more than one circular RNA construct.

Examples of further suitable lipids include the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine.

A lipid nanoparticle composition may optionally comprise one or more coatings. For example, a nanoparticle composition may be formulated in a capsule, film, or tablet having a coating. A capsule, film, or tablet including a composition described herein may have any useful size, tensile strength, hardness, or density.

In one embodiment, the lipid nanoparticles may have a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm. In one embodiment, the lipid nanoparticles may have a diameter from about 10 to 500 nm. In one embodiment, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm. Each possibility represents a separate embodiment.

In some embodiments, a nanoparticle (e.g., a lipid nanoparticle) has a mean diameter of 10-500 nm, 20-400 nm, 30-300 nm, or 40-200 nm. In some embodiments, a nanoparticle (e.g., a lipid nanoparticle) has a mean diameter of 50-150 nm, 50-200 nm, 80-100 nm, or 80-200 nm.

In some embodiments, the lipid nanoparticles described herein can have a diameter from below 0.1 μm to up to 1 mm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, less than 975 μm.

In another embodiment, LNPs may have a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm. Each possibility represents a separate embodiment.

A nanoparticle composition may be relatively homogenous. A polydispersity index may be used to indicate the homogeneity of a nanoparticle composition, e.g., the particle size distribution of the lipid nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition may be from about 0.10 to about 0.20. Each possibility represents a separate embodiment.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about −20 mV to about +20 mV, from about −20 mV to about +15 mV, from about −20 mV to about +10 mV, from about −20 mV to about +5 mV, from about −20 mV to about 0 mV, from about −20 mV to about −5 mV, from about −20 mV to about −10 mV, from about −20 mV to about −15 mV from about −20 mV to about +20 mV, from about −20 mV to about +15 mV, from about −20 mV to about +10 mV, from about −20 mV to about +5 mV, from about −20 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV. Each possibility represents a separate embodiment.

The efficiency of encapsulation of a therapeutic agent describes the amount of therapeutic agent that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of therapeutic agent in a solution containing the lipid nanoparticle composition before and after breaking up the lipid nanoparticle composition with one or more organic solvents or detergents. Fluorescence may be used to measure the amount of free therapeutic agent (e.g., nucleic acids) in a solution. For the lipid nanoparticle compositions described herein, the encapsulation efficiency of a therapeutic agent may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

In some embodiments, the lipid nanoparticle has a polydispersity value of less than 0.4. In some embodiments, the lipid nanoparticle has a net neutral charge at a neutral pH. In some embodiments, the lipid nanoparticle has a mean diameter of 50-200 nm.

The properties of a lipid nanoparticle formulation may be influenced by factors including, but not limited to, the selection of the cationic lipid component, the degree of cationic lipid saturation, the selection of the non-cationic lipid component, the degree of noncationic lipid saturation, the selection of the structural lipid component, the nature of the PEGylation, ratio of all components and biophysical parameters such as size. As described herein, the purity of a PEG lipid component is also important to an LNP's properties and performance.

### F. Methods for Preparing Lipid Nanoparticles (Lnp) Formulations

In one embodiment, a lipid nanoparticle formulation may be prepared by the methods described in International Publication Nos. WO2011127255 or WO2008103276, each of which is herein incorporated by reference in their entirety. In some embodiments, lipid nanoparticle formulations may be as described in International Publication No. WO2019131770, which is herein incorporated by reference in its entirety.

In some embodiments, circular RNA is formulated according to a process described in U.S. patent application Ser. No. 15/809,680. In some embodiments, the present disclosure provides a process of encapsulating circular RNA in transfer vehicles comprising the steps of forming lipids into pre-formed transfer vehicles (i.e. formed in the absence of RNA) and then combining the pre-formed transfer vehicles with RNA. In some embodiments, the novel formulation process results in an RNA formulation with higher potency (peptide or protein expression) and higher efficacy (improvement of a biologically relevant endpoint) both in vitro and in vivo with potentially better tolerability as compared to the same RNA formulation prepared without the step of preforming the lipid nanoparticles (e.g., combining the lipids directly with the RNA).

For certain cationic lipid nanoparticle formulations of RNA, in order to achieve high encapsulation of RNA, the RNA in buffer (e.g., citrate buffer) has to be heated. In those processes or methods, the heating is required to occur before the formulation process (i.e. heating the separate components) as heating post-formulation (post-formation of nanoparticles) does not increase the encapsulation efficiency of the RNA in the lipid nanoparticles. In contrast, in some embodiments of the novel processes of the present disclosure, the order of heating of RNA does not appear to affect the RNA encapsulation percentage. In some embodiments, no heating (i.e. maintaining at ambient temperature) of one or more of the solutions comprising the pre-formed lipid nanoparticles, the solution comprising the RNA and the mixed solution comprising the lipid nanoparticle encapsulated RNA is required to occur before or after the formulation process.

RNA may be provided in a solution to be mixed with a lipid solution such that the RNA may be encapsulated in lipid nanoparticles. A suitable RNA solution may be any aqueous solution containing RNA to be encapsulated at various concentrations. For example, a suitable RNA solution may contain an RNA at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, or 1.0 mg/ml. In some embodiments, a suitable RNA solution may contain an RNA at a concentration in a range from about 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml.

Typically, a suitable RNA solution may also contain a buffering agent and/or salt. Generally, buffering agents can include HEPES, Tris, ammonium sulfate, sodium bicarbonate, sodium citrate, sodium acetate, potassium phosphate or sodium phosphate. In some embodiments, suitable concentration of the buffering agent may be in a range from about 0.1 mM to 100 mM, 0.5 mM to 90 mM, 1.0 mM to 80 mM, 2 mM to 70 mM, 3 mM to 60 mM, 4 mM to 50 mM, 5 mM to 40 mM, 6 mM to 30 mM, 7 mM to 20 mM, 8 mM to 15 mM, or 9 to 12 mM.

Exemplary salts can include sodium chloride, magnesium chloride, and potassium chloride. In some embodiments, suitable concentration of salts in an RNA solution may be in a range from about 1 mM to 500 mM, 5 mM to 400 mM, 10 mM to 350 mM, 15 mM to 300 mM, 20 mM to 250 mM, 30 mM to 200 mM, 40 mM to 190 mM, 50 mM to 180 mM, 50 mM to 170 mM, 50 mM to 160 mM, 50 mM to 150 mM, or 50 mM to 100 mM.

In some embodiments, a suitable RNA solution may have a pH in a range from about 3.5-6.5, 3.5-6.0, 3.5-5.5, 3.5-5.0, 3.5-4.5, 4.0-5.5, 4.0-5.0, 4.0-4.9, 4.0-4.8, 4.0-4.7, 4.0-4.6, or 4.0-4.5.

Various methods may be used to prepare an RNA solution suitable for the present disclosure. In some embodiments, RNA may be directly dissolved in a buffer solution described herein. In some embodiments, an RNA solution may be generated by mixing an RNA stock solution with a buffer solution prior to mixing with a lipid solution for encapsulation. In some embodiments, an RNA solution may be generated by mixing an RNA stock solution with a buffer solution immediately before mixing with a lipid solution for encapsulation.

According to the present disclosure, a lipid solution contains a mixture of lipids suitable to form transfer vehicles for encapsulation of RNA. In some embodiments, a suitable lipid solution is ethanol based. For example, a suitable lipid solution may contain a mixture of desired lipids dissolved in pure ethanol (i.e. 100% ethanol). In another embodiment, a suitable lipid solution is isopropyl alcohol based. In another embodiment, a suitable lipid solution is dimethylsulfoxide-based. In another embodiment, a suitable lipid solution is a mixture of suitable solvents including, but not limited to, ethanol, isopropyl alcohol and dimethylsulfoxide.

A suitable lipid solution may contain a mixture of desired lipids at various concentrations. In some embodiments, a suitable lipid solution may contain a mixture of desired lipids at a total concentration in a range from about 0.1-100 mg/ml, 0.5-90 mg/ml, 1.0-80 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml.

Nanoparticles can be made in a 1 fluid stream or with mixing processes such as microfluidics and T-junction mixing of two fluid streams, one of which contains the circular RNA and the other has the lipid components.

In some embodiments, the lipid nanoparticles described herein may be synthesized using methods comprising, for example, microfluidic mixers, microstructure-induced chaotic advection (MICA), a Slit Interdigital Microstructured Mixer (SIMM-V2) or a Standard Slit Interdigital Micro Mixer (SSIMM) or Caterpillar (CPMM) or Impinging-jet (IJMM) from the Institut fur Mikrotechnik Mainz GmbH, Mainz Germany), using a micromixer chip, and/or using technology. Exemplary mixers and methods are known in the art.

Additional lipid nanoparticle formulations and methods of producing are described in detail in WO2021226597 and WO2021113777, which are incorporated herein by reference in their entireties. For example, disclosed in WO2021226597 and WO2021113777 is a method of preparing lipid nanoparticle formulations of ionizable lipids 128 and 129 of Table 3. Ethanol phase contained ionizable Lipid 128 or Lipid 129 from Table 3, DOPE, Cholesterol, and DSPE-PEG 2000 (Avanti Polar Lipids Inc.) at a weight ratio of 16:1:4:1 or 62:4:33:1 molar ratio combined with an aqueous phase containing circular RNA and 25 mM sodium acetate buffer at pH 5.2. A 3:1 aqueous to ethanol mixing ratio was used. The formulated LNPs were then dialyzed in 1 L of water and exchanged 2 times over 18 hours. Dialyzed LNPs were filtered using 0.2 μm filter. Prior to in vivo dosing, LNPs were diluted in PBS. LNP sizes were determined by dynamic light scattering. A cuvette with 1 mL of 20 μg/mL LNPs in PBS (pH 7.4) was measured for Z-average using the Malvern Panalytical Zetasizer Pro. The Z-average and polydispersity index were recorded

### G. Other Delivery Vehicles Known in the Art

In certain embodiments, other delivery vehicles that are known in the art may be used to transport the circular RNA (i.e., are transfer vehicles encompassed herein).

In some embodiments, liposomes or other lipid bilayer vesicles may be used as a component or as the whole transfer vehicle to facilitate or enhance the delivery and release of circular RAN to one or more target cells. Liposomes are usually characterized by having an interior space sequestered from an outer medium by a membrane of one or more bilayers forming a microscopic sack or vesicle. Bilayer membranes of liposomes are typically formed by lipids, i.e. amphiphilic molecules of synthetic or natural origin that comprise spatially separated hydrophobic or hydrophilic domains (Lasic, D, and Papahadjopoulos, D., eds. Medical Applications of Liposomes. Elsevier, Amsterdam, 1998).

In certain embodiments, the transfer vehicle for transporting the circular RNA comprises a dendrimer. Use of “dendrimer” describes the architectural motif of the transfer vehicle. In some embodiments, the dendrimer includes but is not limited to containing an interior core and one or more layers (i.e. generations) that extend or attach out from the interior core. In some of the embodiments, the generations may contain one or more branching points and an exterior surface of terminal groups that attach to the outermost generation. The branching points, in certain embodiments, may be mostly monodispersed and contain symmetric branching units built around the interior core. In some embodiments, the interior core. Synthesis of the dendrimer may comprise the divergent method, convergent growth, hypercore and branched monomer growth, double exponential growth, lego chemistry, click chemistry and other methods as available in the art (Mendes L. et al., Molecules. 2017. 22 (9): 1401 further describes these methods).

In certain embodiments, as described herein, the transfer vehicle for the circular RNA construct comprises a polymer nanoparticle. In some embodiments, the polymer nanoparticle includes nanocapsules and nanospheres. Nanocapsules, in some embodiments, are composed of an oily core surrounded by a polymeric shell. In some embodiments, the circular RNA is contained within the core and the polymeric shell controls the release of the circular RNA. On the other hand, nanospheres comprise a continuous polymeric network in which the circular RNA is retained or absorbed onto the surface. In some embodiments, cationic polymers are used to encapsulate the circular RNA due to the favorable electrostatic interaction of the cations to the negatively charged nucleic acids and cell membrane. The polymer nanoparticle may be prepared by various methods. In some embodiments, the polymer nanoparticle may be prepared by nanoprecipitation, emulsion techniques, solvent evaporation, solvent diffusion, reverse salting-out or other methods available in the art.

In certain embodiments, as described herein, the transfer vehicle for the circular RNA construct comprises a polymer-lipid hybrid nanoparticle (LPHNP). In some embodiments, the LPHNP comprises a polymer core enveloped within a lipid bilayer. In some embodiments, the polymer core encapsulates the circular RNA construct. In some embodiments, the LPHNP further comprises an outer lipid bilayer. In certain embodiments this outer lipid bilayer comprises a PEG-lipid, helper lipid, cholesterol or other molecule as known in the art to help with stability in a lipid-based nanoparticle. The lipid bilayer closest to the polymer core mitigates the loss of the entrapped circular RNA during LPHNP formation and protects from degradation of the polymer core by preventing diffusion of water from outside of the transfer vehicle into the polymer core (Mukherjee et al., In J. Nanomedicine. 2019; 14: 1937-1952).

In certain embodiments, the circular RNA can be transported using a peptide-based delivery mechanism. In some embodiments, the peptide-based delivery mechanism comprises a lipoprotein. Based on the size of the drug to be delivered, the lipoprotein may be either a low-density (LDL) or high-density lipoprotein (HDL). As seen in U.S. Pat. No. 8,734,853B2, high-density lipoproteins are capable of transporting a nucleic acid in vivo and in vitro. In particular embodiments, the lipid component includes cholesterol. In more particular embodiments, the lipid component includes a combination of cholesterol and cholesterol oleate.

In certain embodiments, the circular RNA construct can be transported using a carbohydrate carrier or a sugar-nanocapsule. In certain embodiments, the carbohydrate carrier comprises a sugar-decorated nanoparticle, peptide- and saccharide-conjugated dendrimer, nanoparticles based on polysaccharides, and other carbohydrate-based carriers available in the art. As described herein, the incorporation of carbohydrate molecules may be through synthetic means. In some embodiments, the carbohydrate carrier comprises polysaccharides. These polysaccharides may be made from the microbial cell wall of the target cell. For example, carbohydrate carriers comprised of mannan carbohydrates have been shown to successfully deliver mRNA (Son et al., Nano Lett. 2020. 20(3): 1499-1509).

In certain embodiments, as provided herein, the transfer vehicle for the circular RNA is a glyconanoparticle (GlycoNP). As known in the art, glyconanoparticles comprise a core comprising gold, iron oxide, semiconductor nanoparticles or a combination thereof. In some embodiments, the glyconanoparticle is functionalized using carbohydrates. In certain embodiments, the glyconanoparticle comprises a carbon nanotube or graphene. In one embodiment the glyconanoparticle comprises a polysaccharide-based GlycoNP (e.g., chitosan-based GlycoNP). In certain embodiments, the glyconanoparticle is a glycodendrimer.

In certain embodiments, as provided herein, the circular RNA is transferred through use of an exosome, a type of extracellular vesicle. Exosomes naturally are secreted by various types of cells and are used as a transport vesicle for various forms of cargo. During delivery exosomes can contain and protect specific mRNAs, regulatory microRNAs, lipids, and proteins (Luan et al., Acta Pharmacologica Sinica. 2017. 38:754-763). Naturally, exosomes may be 30 nm to 125 nm.

In some embodiments, the exosome may be made in part from an immune cell. As shown in Haney et al, use of immune cell derived exosomes are able to avoid mononuclear phagocytes (J Control Release. 2015. 207:18-30). In some embodiments, the exosome may be a dendritic cell, macrophage, T-cell, B-cell or derived from another immune cell. As seen in WO/2021/041473A1, various forms of RNAs of varying lengths may be transported through exosome delivery including messenger RNA (mRNA), microRNA (miRNA), long intergenic non-coding RNA (lincRNA), long non-coding RNA (lncRNA), non-coding RNA (ncRNA), non-messenger RNA (nmRNA), small RNA (sRNA), small non-messenger RNA (smnRNA), DNA damage response RNA (DD RNA), extracellular RNA (exRNA), small nuclear RNA (snRNA), small nucleolar RNA (snoRNA), and precursor messenger RNA (pre-mRNA).

In other embodiments, the transfer vehicle may comprise in whole or in part from a fusome. In some embodiments, the fusome is derived from an endoplasmic reticulum of a germline cyst. In certain embodiments, the germline cyst is from a Drosophila ovary.

In certain embodiments, the circular RNA construct may be transported using noncellular and instead be through mechanical delivery mechanisms. In some embodiments, this delivery method includes microneedles, electroporation, continuous pumps and/or gene guns.

In some embodiments, the transfer vehicle of the circular RNA construct is a solution or diluent comprising of a salt or a buffer.

### H. Targeting

In some embodiments, the compositions use targeting moieties that may be bound (either covalently or non-covalently) to the transfer vehicles to encourage localization of such transfer vehicle at certain target cells or target tissues. For example, targeting may be mediated by the inclusion of one or more endogenous targeting moieties in or on the transfer vehicle to encourage distribution to the target cells or tissues. Recognition of the targeting moiety by the target tissues actively facilitates tissue distribution and cellular uptake of the transfer vehicle and/or its contents in the target cells and tissues (e.g., the inclusion of an apolipoprotein-E targeting ligand in or on the transfer vehicle encourages recognition and binding of the transfer vehicle to endogenous low density lipoprotein receptors expressed by hepatocytes).

As provided herein, the composition can comprise a moiety capable of enhancing affinity of the composition to the target cell. Targeting moieties may be linked to the outer bilayer of the lipid particle during formulation or post-formulation. These methods are well known in the art. In addition, some lipid particle formulations may employ fusogenic polymers such as PEAA, hemagluttinin, other lipopeptides (see U.S. patent application Ser. No. 08/835,281, and 60/083,294, which are incorporated herein by reference) and other features useful for in vivo and/or intracellular delivery. In other some embodiments, the compositions demonstrate improved transfection efficacies, and/or demonstrate enhanced selectivity towards target cells or tissues of interest. Contemplated therefore are compositions which comprise one or more moieties (e.g., peptides, aptamers, oligonucleotides, a vitamin or other molecules) that are capable of enhancing the affinity of the compositions and their nucleic acid contents for the target cells or tissues. Suitable moieties may optionally be bound or linked to the surface of the transfer vehicle. In some embodiments, the targeting moiety may span the surface of a transfer vehicle or be encapsulated within the transfer vehicle. Suitable moieties and are selected based upon their physical, chemical or biological properties (e.g., selective affinity and/or recognition of target cell surface markers or features). Cell-specific target sites and their corresponding targeting ligand can vary widely. Suitable targeting moieties are selected such that the unique characteristics of a target cell are exploited, thus allowing the composition to discriminate between target and non-target cells. For example, in some embodiments, compositions may include surface markers (e.g., apolipoprotein-B or apolipoprotein-E) that selectively enhance recognition of, or affinity to hepatocytes (e.g., by receptor-mediated recognition of and binding to such surface markers). As an example, the use of galactose as a targeting moiety would be expected to direct the compositions to parenchymal hepatocytes, or alternatively the use of mannose containing sugar residues as a targeting ligand would be expected to direct the compositions to liver endothelial cells (e.g., mannose containing sugar residues that may bind preferentially to the asialoglycoprotein receptor present in hepatocytes). (See Hillery A M, et al. “Drug Delivery and Targeting: For Pharmacists and Pharmaceutical Scientists” (2002) Taylor & Francis, Inc.) The presentation of such targeting moieties that have been conjugated to moieties present in the transfer vehicle (e.g., a lipid nanoparticle) therefore facilitate recognition and uptake of the compositions in target cells and tissues. Examples of suitable targeting moieties include one or more peptides, proteins, aptamers, vitamins and oligonucleotides.

In particular embodiments, a transfer vehicle comprises a targeting moiety. In some embodiments, the targeting moiety mediates receptor-mediated endocytosis selectively into a specific population of cells or tissue. In some embodiments, the targeting moiety is capable of binding to a T cell antigen. In some embodiments, the targeting moiety is capable of binding to a NK, NKT, or macrophage antigen. In some embodiments, the targeting moiety is capable of binding to a protein selected from the group CD3, CD4, CD8, PD-1, 4-1BB, and CD2. In some embodiments, the targeting moiety is a single chain Fv (scFv) fragment, nanobody, peptide, peptide-based macrocycle, minibody, heavy chain variable region, light chain variable region or fragment thereof. In some embodiments, the targeting moiety is selected from T-cell receptor motif antibodies, T-cell a chain antibodies, T-cell R chain antibodies, T-cell 7 chain antibodies, T-cell 6 chain antibodies, CCR7 antibodies, CD3 antibodies, CD4 antibodies, CD5 antibodies, CD7 antibodies, CD8 antibodies, CD11b antibodies, CD11c antibodies, CD16 antibodies, CD19 antibodies, CD20 antibodies, CD21 antibodies, CD22 antibodies, CD25 antibodies, CD28 antibodies, CD34 antibodies, CD35 antibodies, CD40 antibodies, CD45RA antibodies, CD45RO antibodies, CD52 antibodies, CD56 antibodies, CD62L antibodies, CD68 antibodies, CD80 antibodies, CD95 antibodies, CD117 antibodies, CD127 antibodies, CD133 antibodies, CD137 (4-1BB) antibodies, CD163 antibodies, F4/80 antibodies, IL-4Rα antibodies, Sca-1 antibodies, CTLA-4 antibodies, GITR antibodies GARP antibodies, LAP antibodies, granzyme B antibodies, LFA-1 antibodies, transferrin receptor antibodies, and fragments thereof. In some embodiments, the targeting moiety is a small molecule binder of an ectoenzyme on lymphocytes. Small molecule binders of ectoenzymes include A2A inhibitors CD73 inhibitors, CD39 or adesines receptors A2aR and A2bR. Potential small molecules include AB928.

In some embodiments, transfer vehicles are formulated and/or targeted as described in Shobaki N, Sato Y, Harashima H. Mixing lipids to manipulate the ionization status of lipid nanoparticles for specific tissue targeting. Int J Nanomedicine. 2018; 13:8395-8410. Published 2018 Dec. 10. In some embodiments, a transfer vehicle is made up of 3 lipid types. In some embodiments, a transfer vehicle is made up of 4 lipid types. In some embodiments, a transfer vehicle is made up of 5 lipid types. In some embodiments, a transfer vehicle is made up of 6 lipid types.

In some embodiments, the target cells are deficient in a protein or enzyme of interest. In some embodiments, the compositions of the present disclosure transfect the target cells on a discriminatory basis (i.e., do not transfect non-target cells). The compositions of the present disclosure may also be prepared to preferentially target a variety of target cells, which include, but are not limited to, immune cells, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

The compositions of the present disclosure may be prepared to preferentially distribute to target cells, including but not limited to the heart, lungs, kidneys, liver, and spleen, ocular, or cells in the central nervous system. In some embodiments, the compositions of the present disclosure distribute into the cells of the liver or spleen to facilitate the delivery and the subsequent expression of the circRNA comprised therein by the cells of the liver (e.g., hepatocytes) or the cells of spleen (e.g., immune cells). The targeted cells may function as a biological “reservoir” or “depot” capable of producing, and systemically excreting a functional protein or enzyme.

In some embodiments, the transfer vehicles comprise circRNA which encode a deficient protein or enzyme. Upon distribution of such compositions to the target tissues and the subsequent transfection of such target cells, the exogenous circRNA loaded into the transfer vehicle (e.g., a lipid nanoparticle) may be translated in vivo to produce a functional protein or enzyme encoded by the exogenously administered circRNA (e.g., a protein or enzyme in which the subject is deficient). Accordingly, the compositions of the present disclosure exploit a subject's ability to translate exogenously- or recombinantly-prepared circRNA to produce an endogenously-translated protein or enzyme, and thereby produce (and where applicable excrete) a functional protein or enzyme. The expressed or translated proteins or enzymes may also be characterized by the in vivo inclusion of native post-translational modifications which may often be absent in recombinantly-prepared proteins or enzymes, thereby further reducing the immunogenicity of the translated protein or enzyme.

### 6. Pharmaceutical Compositions

In certain embodiments, provided herein are compositions (e.g., pharmaceutical compositions) comprising a therapeutic agent provided herein. In some embodiments, the therapeutic agent is a circular RNA polynucleotide provided herein. In some embodiments the therapeutic agent is a vector provided herein. In some embodiments, the therapeutic agent is a cell comprising a circular RNA, a precursor polynucleotide, or vector provided herein (e.g., a human cell, such as a human T cell).

In certain embodiments, the composition further comprises a pharmaceutically acceptable carrier or excipient. In some embodiments, and as described elsewhere herein, the pharmaceutical composition comprises at least one circular RNA polynucleotide and a transfer vehicle. In some embodiments, the transfer vehicle comprises at least one of (i) an ionizable lipid, (ii) a structural lipid, and (iii) a PEG-modified lipid.

In some embodiments, the transfer vehicle is a nanoparticle or lipid nanoparticle. In some embodiments, the nanoparticle is a lipid nanoparticle (LNP), a core-shell nanoparticle, a biodegradable nanoparticle, a biodegradable lipid nanoparticle, a polymer nanoparticle, a polyplex or a biodegradable polymer nanoparticle.

In some embodiments, the pharmaceutical composition comprises a targeting moiety. The targeting moiety mediates receptor-mediated endocytosis, endosome fusion, or direct fusion into selected cells of a selected cell population or tissue in the absence of cell isolation or purification. In some embodiments, the pharmaceutical composition comprises a targeting moiety operably connected to the nanoparticle. In some embodiments, the targeting moiety is a small molecule, scFv, nanobody, peptide, cyclic peptide, di or tri cyclic peptide, minibody, polynucleotide aptamer, engineered scaffold protein, heavy chain variable region, light chain variable region, or a fragment thereof. In some embodiments, less than 1%, by weight, of the polynucleotides in the composition are double stranded RNA, DNA splints, DNA template, or triphosphorylated RNA. In some embodiments, less than 1%, by weight, of the polynucleotides and proteins in the pharmaceutical composition are double stranded RNA, DNA splints, DNA template, triphosphorylated RNA, phosphatase proteins, protein ligases, RNA polymerases, and capping enzymes.

In some embodiments, the compositions provided herein comprise a therapeutic agent provided herein in combination with other pharmaceutically active agents or drugs, such as anti-inflammatory drugs or antibodies capable of targeting B cell antigens, e.g., anti-CD20 antibodies, e.g., rituximab.

With respect to pharmaceutical compositions, the pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the therapeutic agent(s) and one which has no detrimental side effects or toxicity under the conditions of use.

The choice of carrier will be determined in part by the particular therapeutic agent, as well as by the particular method used to administer the therapeutic agent. Accordingly, there are a variety of suitable formulations of the pharmaceutical compositions provided herein. In certain embodiments, the pharmaceutical composition comprises a preservative. In some embodiments, the pharmaceutical composition comprises a buffering agent.

In some embodiments, the concentration of therapeutic agent in the pharmaceutical composition can vary, e.g., less than about 1%, or at least about 1%, 2%, 3%, 4%, 5%, 6%, 7%, 8%, 9% 10%, 15%, 20%, 25%, 30%, 35%, 40%, 45%, or about 50% or more by weight, and can be selected primarily by fluid volumes, and viscosities, in accordance with the particular mode of administration selected.

Formulations for oral, aerosol, parenteral (e.g., subcutaneous, intravenous, intraarterial, intramuscular, intradermal, intraperitoneal, and intrathecal), and topical administration are known in the art. More than one route can be used to administer the therapeutic agents provided herein, and in certain instances, a particular route can provide a more immediate and more effective response than another route.

In certain embodiments, the therapeutic agents provided herein can be formulated as inclusion complexes, such as cyclodextrin inclusion complexes, or LNPs or liposomes.

In some embodiments, the composition comprises a precursor RNA polynucleotide described herein, a polynucleotide described herein, a circular RNA polynucleotide described herein, or combinations thereof; and a transfer vehicle described herein. In some embodiments, the transfer vehicle comprises at least one of (i) an ionizable lipid, (ii) a structural lipid, and (iii) a PEG-modified lipid. In some embodiments, the composition further comprises a targeting moiety.

In some embodiments, the therapeutic agents provided herein are formulated in time-released, delayed release, or sustained release delivery systems such that the delivery of the composition occurs prior to, and with sufficient time to, cause sensitization of the site to be treated. Such systems can avoid repeated administrations of the therapeutic agent, thereby increasing convenience to the subject and the physician, and may be particularly suitable for certain composition embodiments provided herein. In one embodiment, the compositions of the present disclosure are formulated such that they are suitable for extended-release of the circRNA contained therein. Such extended-release compositions may be conveniently administered to a subject at extended dosing intervals. For example, in one embodiment, the compositions of the present disclosure are administered to a subject twice a day, daily or every other day. In an embodiment, the compositions of the present disclosure are administered to a subject twice a week, once a week, every ten days, every two weeks, every three weeks, every four weeks, once a month, every six weeks, every eight weeks, every three months, every four months, every six months, every eight months, every nine months or annually.

In some embodiments, a protein encoded by a polynucleotide is produced by a target cell for sustained amounts of time. For example, the protein may be produced for more than one hour, more than four, more than six, more than 12, more than 24, more than 48 hours, or more than 72 hours after administration. In some embodiments the polypeptide is expressed at a peak level about six hours after administration. In some embodiments the expression of the polypeptide is sustained at least at a therapeutic level. In some embodiments, the polypeptide is expressed at least at a therapeutic level for more than one, more than four, more than six, more than 12, more than 24, more than 48, or more than 72 hours after administration. In some embodiments, the polypeptide is detectable at a therapeutic level in patient tissue (e.g., liver or lung). In some embodiments, the level of detectable polypeptide is from continuous expression from the circRNA composition over periods of time of more than one, more than four, more than six, more than 12, more than 24, more than 48, or more than 72 hours after administration.

In certain embodiments, a protein encoded by a polynucleotide is produced at levels above normal physiological levels. The level of protein may be increased as compared to a control. In some embodiments, the control is the baseline physiological level of the polypeptide in a normal individual or in a population of normal individuals. In other embodiments, the control is the baseline physiological level of the polypeptide in an individual having a deficiency in the relevant protein or polypeptide or in a population of individuals having a deficiency in the relevant protein or polypeptide. In some embodiments, the control can be the normal level of the relevant protein or polypeptide in the individual to whom the composition is administered. In other embodiments, the control is the expression level of the polypeptide upon other therapeutic intervention, e.g., upon direct injection of the corresponding polypeptide, at one or more comparable time points.

In certain embodiments, the levels of a protein encoded by a polynucleotide are detectable at 3 days, 4 days, 5 days, or 1 week or more after administration. Increased levels of protein may be observed in a tissue (e.g., liver or lung).

In some embodiments, the method yields a sustained circulation half-life of a protein encoded by a polynucleotide. For example, the protein may be detected for hours or days longer than the half-life observed via subcutaneous injection of the protein or mRNA encoding the protein. In some embodiments, the half-life of the protein is 1 day, 2 days, 3 days, 4 days, 5 days, or 1 week or more.

In some embodiments, the modified polynucleotide described herein is a circular RNA that affects net charge, and therefore may be suitable for use with a delivery or transfer vehicle comprising an ionizable lipid.

Different types of release delivery systems are available and known to those of ordinary skill in the art. See, e.g., U.S. Pat. Nos. 5,075,109, 4,452,775, 4,667,014, 4,748,034, and 5,239,660, 3,832,253 and 3,854,480. In some embodiments, the therapeutic agent can be conjugated either directly or indirectly through a linking moiety to a targeting moiety. Methods for conjugating therapeutic agents to targeting moieties is known in the art. See, e.g., Wadwa et al., J, Drug Targeting 3:111 (1995) and U.S. Pat. No. 5,087,616. In some embodiments, the therapeutic agents provided herein are formulated into a depot form, such that the manner in which the therapeutic agent is released into the body to which it is administered is controlled with respect to time and location within the body (see, for example, U.S. Pat. No. 4,450,150).

In certain embodiments, the compositions may be loaded with diagnostic radionuclide, fluorescent materials or other materials that are detectable in both in vitro and in vivo applications. For example, suitable diagnostic materials may include Rhodamine-dioleoylphosphatidylethanolamine (Rh-PE), Green Fluorescent Protein circRNA (GFP circRNA), Renilla Luciferase circRNA and Firefly Luciferase circRNA.

### 7. Therapeutic Methods

Provided herein are methods of treating a subject in need thereof comprising administering a therapeutically effective amount of the circular RNA provided herein and/or a composition comprising the circular RNA provided herein. Provided herein are also methods of preventing a disease or disorder in a subject in need thereof comprising a therapeutically effective amount of circular RNA provided herein and/or a composition comprising the circular RNA provided herein. In some embodiments, in addition to the circular RNA, a delivery vehicle, and optionally, a targeting moiety operably connected to the delivery vehicle is administered.

In certain aspects, provided herein is a method of producing a protein of interest in a subject in need thereof by introducing or administering a pharmaceutical composition comprising a circular RNA, described herein.

In certain embodiments, the therapeutic agents provided herein (e.g., circular RNA and/or composition comprising the circular RNA) are co-administered with one or more additional therapeutic agents (e.g., in the same pharmaceutical composition or in separate pharmaceutical compositions). In some embodiments, the therapeutic agent provided herein can be administered first and the one or more additional therapeutic agents can be administered second, or vice versa. Alternatively, the therapeutic agent provided herein and the one or more additional therapeutic agents can be administered simultaneously.

In some embodiments, the subject is a mammal. In some embodiments, the mammal referred to herein can be any mammal, including, but not limited to, mammals of the order Rodentia, such as mice and hamsters, or mammals of the order Logomorpha, such as rabbits. The mammals may be from the order Carnivora, including Felines (cats) and Canines (dogs). The mammals may be from the order Artiodactyla, including Bovines (cows) and Swines (pigs), or of the order Perssodactyla, including Equines (horses). The mammals may be of the order Primates, Ceboids, or Simoids (monkeys) or of the order Anthropoids (humans and apes). Preferably, the mammal is a human.

In some embodiments, provided herein is a method of vaccinating a subject by introducing or administering the circular RNA construct provided herein and/or a composition comprising the circular RNA construct provided herein.

In some embodiments, provided herein is a method of treating an autoimmune disorder in a subject by introducing or administering the circular RNA construct provided herein and/or a composition comprising the circular RNA construct provided herein. In these embodiments, a circular RNA vaccine comprises one or more circular RNA polynucleotides, which encode one or more wild type or engineered proteins, peptides or polypeptides (e.g., antigens, adjuvant, or adjuvant-like proteins). In some embodiments, the one or more circular RNA polynucleotide encodes an antigen or adjuvant derived from an infectious agent. In some embodiments the infectious agent from which the antigen or adjuvant is derived or engineered includes, but is not limited to a virus, bacterium, fungus, protozoan, and/or parasite. In some embodiments, the antigen is a viral antigen. In an embodiment, the antigen is a SARS-CoV-2 antigen. In an embodiment, the antigen is SARS-CoV-2 spike protein. In an embodiment, the antigen is selected from or derived from the group consisting of rotavirus, foot and mouth disease virus, influenza A virus, influenza B virus, influenza C virus, H1N1, H2N2, H3N2, H5N1, H7N7, H1N2, H9N2, H7N2, H7N3, H10N7, human parainfluenza type 2, herpes simplex virus, Epstein-Barr virus, varicella virus, porcine herpesvirus 1, cytomegalovirus, lyssavirus, Bacillus anthracis, anthrax PA and derivatives, poliovirus, Hepatitis A, Hepatitis B, Hepatitis C, Hepatitis E, distemper virus, venezuelan equine encephalomyelitis, feline leukemia virus, reovirus, respiratory syncytial virus, Lassa fever virus, polyoma tumor virus, canine parvovirus, papilloma virus, tick borne encephalitis virus, rinderpest virus, human rhinovirus species, Enterovirus species, Mengovirus, paramyxovirus, avian infectious bronchitis virus, human T-cell leukemia-lymphoma virus 1, human immunodeficiency virus-1, human immunodeficiency virus-2, lymphocytic choriomeningitis virus, parvovirus B19, adenovirus, rubella virus, yellow fever virus, dengue virus, bovine respiratory syncitial virus, corona virus, Bordetella pertussis, Bordetella bronchiseptica, Bordetella parapertussis, Brucella abortis, Brucella melitensis, Brucella suis, Brucella ovis, Brucella species, Escherichia coli, Salmonella species, Salmonella typhi, Streptococci, Vibrio cholera, Vibrio parahaemolyticus, Shigella, Pseudomonas, tuberculosis, avium, Bacille Calmette Guerin, Mycobacterium leprae, Pneumococci, Staphlylococci, Enterobacter species, Rochalimaia henselae, Pasteurella haemolytica, Pasteurella multocida, Chlamydia trachomatis, Chlamydia psittaci, Lymphogranuloma venereum, Treponema pallidum, Haemophilus species, Mycoplasma bovigenitalium, Mycoplasma pulmonis, Mycoplasma species, Borrelia burgdorferi, Legionalla pneumophila, Colstridium botulinum, Corynebacterium diphtheriae, Yersinia entercolitica, Rickettsia rickettsii, Rickettsia typhi, Rickettsia prowsaekii, Ehrlichia chaffeensis, Anaplasma phagocytophilum, Plasmodium falciparum, Plasmodium vivax, Plasmodium malariae, Schistosomes, trypanosomes, Leishmania species, Filarial nematodes, trichomoniasis, sarcosporidiasis, Taenia saginata, Taenia solium, Leishmania, Toxoplasma gondii, Trichinella spiralis, coccidiosis, Eimeria tenella, Cryptococcus neoformans, Candida albican, Aspergillus fumigatus, coccidioidomycosis, Neisseria gonorrhoeae, malaria circumsporozoite protein, malaria merozoite protein, trypanosome surface antigen protein, pertussis, alphaviruses, adenovirus, diphtheria toxoid, tetanus toxoid, meningococcal outer membrane protein, streptococcal M protein, Influenza hemagglutinin, cancer antigen, tumor antigens, toxins, Clostridium perfringens epsilon toxin, ricin toxin, pseudomonas exotoxin, exotoxins, neurotoxins, cytokines, cytokine receptors, monokines, monokine receptors, plant pollens, animal dander, and dust mites. In some embodiments, the adjuvant is selected from or derived from the group consisting of BCSP31, MOMP, FomA, MymA, ESAT6, PorB, PVL, Porin, OmpA, PepO, OmpU, Lumazine synthase, Omp16, Omp19, CobT, RpfE, Rv0652, HBHA, NhhA, DnaJ, Pneumolysin, Falgellin, IFN-alpha, IFN-gamma, IL-2, IL-12, IL-15, IL-18, IL-21, GM-CSF, IL-lb, IL-6, TNF-a, IL-7, IL-17, IL-1Beta, anti-CTLA4, anti-PD1, anti-41BB, PD-L1, Tim-3, Lag-3, TIGIT, GITR, and anti-CD3.

In some embodiments, provided herein is a method of treating cancer in a subject by introducing or administering the circular RNA construct provided herein and/or a composition comprising the circular RNA construct provided herein.

In some embodiments, the circular RNA construct encodes a CAR, the CARs have biological activity, e.g., ability to recognize an antigen, e.g., CD19, HER2, or BCMA, such that the CAR, when expressed by a cell, is able to mediate an immune response against the cell expressing the antigen, e.g., CD19, HER2, or BCMA, for which the CAR is specific. Thus, in certain embodiments, provided herein are methods of treating and/or preventing a disease in a subject (e.g., mammalian subject, such as a human subject). Without being bound to a particular theory or mechanism, where the circular RNA encodes a CAR, the CARs have biological activity, e.g., ability to recognize an antigen, e.g., CD19 or BCMA, such that the CAR, when expressed by a cell, is able to mediate an immune response against the cell expressing the antigen, e.g., CD19 or BCMA, for which the CAR is specific. In this regard, an embodiment provided herein provides a method of treating or preventing an autoimmune disease in a subject, comprising administering to the subject the circular RNA therapeutic agents, and/or the pharmaceutical compositions provided herein in an amount effective to treat or prevent autoimmune disease in the subject.

In some embodiments, the subject has an autoimmune disease or disorder.

Adoptive T-cell immunotherapy is a rapidly growing field, in particular in cancer treatments. In general, chimeric antigen receptor (CAR) T cell or “CAR-T” engagement of CD19-expressing cancer cells results in T-cell activation, proliferation and secretion of inflammatory cytokines and chemokines resulting in tumor cell lysis. However, while CAR-T therapies have become an important tool in cancer treatments, they have toxic side effects and involve complex procedures. Treatment with CAR-T can lead to a large and rapid release of cytokines into the blood and can cause cytokine release syndrome (CRS) or CAR-T cell-related encephalopathy syndrome (CRES), also referred to as neurotoxicity associated with CAR-T. CRS is the most common and well-described toxicity associated with CAR-T therapy, occurring in over 90% of patients at any grade and is characterized by high fever, hypotension, hypoxia and/or multiple organ toxicity and can lead to death. Neurotoxicity is characterized by damage to nervous tissue that can cause tremors, encephalopathy, dizziness or seizures. Additionally, prior to infusion, the patients generally undergo lymphodepletion. Lymphodepletion is known to increase CAR-T cell expansion and enhanced efficacy of infused CAR-T cells by, for example, altering the tumor phenotype and microenvironment. However, lymphodepletion agents often cause side effects to the patients. For example, lymphodepletion can cause neutropenia, anemia, thrombocytopenia, and immunosuppression, leading to a greater risk of infection, along with other toxicities. In addition to the toxicities associated with targeted CAR-T therapies, there are procedures, specialized equipment, and costs involved in producing the modified lymphocytes. CAR-T therapies require an assortment of protocols to isolate, genetically modify, and selectively expand the redirected cells before infusing them back into the patient.

In some embodiments, the subject has a cancer selected from the group consisting of: acute myeloid leukemia (AML); alveolar rhabdomyosarcoma; B cell malignancies; bladder cancer (e.g., bladder carcinoma); bone cancer; brain cancer (e.g., medulloblastoma and glioblastoma multiforme); breast cancer; cancer of the anus, anal canal, or anorectum; cancer of the eye; cancer of the intrahepatic bile duct; cancer of the joints; cancer of the neck; gallbladder cancer; cancer of the pleura; cancer of the nose, nasal cavity, or middle ear; cancer of the oral cavity; cancer of the vulva; chronic lymphocytic leukemia; chronic myeloid cancer; colon cancer; esophageal cancer, cervical cancer; fibrosarcoma; gastrointestinal carcinoid tumor; head and neck cancer (e.g., head and neck squamous cell carcinoma); Hodgkin lymphoma; hypopharynx cancer; kidney cancer; larynx cancer; leukemia; liquid tumors; lipoma; liver cancer; lung cancer (e.g., non-small cell lung carcinoma, lung adenocarcinoma, and small cell lung carcinoma); lymphoma; mesothelioma; mastocytoma; melanoma; multiple myeloma; nasopharynx cancer; non-Hodgkin lymphoma; B-chronic lymphocytic leukemia; hairy cell leukemia; Burkitt's lymphoma; ovarian cancer; pancreatic cancer; cancer of the peritoneum; cancer of the omentum; mesentery cancer; pharynx cancer; prostate cancer; rectal cancer; renal cancer; skin cancer; small intestine cancer; soft tissue cancer; solid tumors; synovial sarcoma; gastric cancer; teratoma; testicular cancer; thyroid cancer; and ureter cancer.

In some embodiments, the subject has an autoimmune disorder selected from scleroderma, Grave's disease, Crohn's disease, Sjogren's disease, multiple sclerosis, Hashimoto's disease, psoriasis, myasthenia gravis, autoimmune polyendocrinopathy syndromes, Type I diabetes mellitus (TIDM), autoimmune gastritis, autoimmune uveoretinitis, polymyositis, colitis, thyroiditis, and the generalized autoimmune diseases typified by human Lupus.

### EMBODIMENTS

Accordingly, the following non-limiting embodiments are provided:

Embodiment 1. A precursor RNA polynucleotide comprising:
(a) a 5′ combined accessory element, comprising:
   (i) a 3′ permuted intron segment comprising a 5′ nucleotide of a 3′ splice site dinucleotide; and
   (ii) a 3′ exon segment comprising a 3′ nucleotide of a 3′ splice site dinucleotide;
(b) an intervening region; and
(c) a 3′ combined accessory element, comprising:
   (i) a 5′ exon segment comprising a 5′ nucleotide of a 5′ splice site dinucleotide; and
   (ii) a 5′ permuted intron segment comprising a 3′ nucleotide of a 5′ splice site dinucleotide;
wherein the 5′ nucleotide of a 3′ splice site dinucleotide, 3′ nucleotide of a 3′ splice site dinucleotide, 5′ nucleotide of a 5′ splice site dinucleotide and 3′ nucleotide of a 5′ splice site dinucleotide each comprise a portion of a sequence or a sequence selected from SEQ ID NOS: 2990-3668.

Embodiment 2. The precursor RNA polynucleotide of embodiment 1, wherein elements (a) (i) through (c) (ii) are arranged in the sequence (a) (i) through (c) (ii); and wherein element (a) is 5′ to element (b); and element (b) is 5′ to element (c).

Embodiment 3. The precursor RNA polynucleotide of embodiment 1, further comprising at least one affinity tag.

Embodiment 4. The precursor RNA polynucleotide of embodiment 3, wherein the 5′ combined accessory element comprises a 5′ affinity tag, and wherein the 5′ affinity tag is located 5′ to the 3′ permuted intron segment.

Embodiment 5. The precursor RNA polynucleotide of embodiment 3 or 4, wherein the 3′ combined accessory element comprises a 3′ affinity tag, and wherein the 3′ affinity tag is located 3′ to the 5′ permuted intron segment.

Embodiment 6. The precursor RNA polynucleotide of any one of embodiments 1-5, wherein the 5′ combined accessory element comprises a 5′ external spacer, and wherein the 5′ external spacer is located 5′ to the 3′ permuted intron segment.

Embodiment 7. The precursor RNA polynucleotide of any one of embodiments 1-6, wherein the 3′ combined accessory element comprises a 3′ external spacer, and wherein the 3′ external spacer is located 3′ to the 5′ permuted intron segment.

Embodiment 8. The precursor RNA polynucleotide of any one of embodiments 1-7, wherein the 5′ combined accessory element comprises a 5′ internal spacer, and wherein the 5′ internal spacer is located 3′ to the 3′ exon segment.

Embodiment 9. The precursor RNA polynucleotide of any one of embodiments 1-8, wherein the 3′ combined accessory element comprises a 3′ internal spacer, and wherein the 3′ internal spacer is located 5′ to the 5′ exon segment.

Embodiment 10. The precursor RNA polynucleotide of any one of embodiments 1-9, wherein the 5′ combined accessory element comprises a 5′ internal duplex sequence located 3′ to the 3′ exon segment, and wherein the 3′ combined accessory element comprises a 3′ internal duplex sequence located 5′ to the 5′ exon segment.

Embodiment 11. The precursor RNA polynucleotide of any one of embodiments 1-10, wherein the 5′ combined accessory element comprises a 5′ external duplex sequence located 5′ to the 3′ permuted intron segment, and wherein the 3′ combined accessory element comprises a 3′ external duplex sequence located 3′ to the 5′ permuted intron segment.

Embodiment 12. The precursor RNA polynucleotide of embodiment 2, wherein element (a)(i) is located 5′ to element (a)(ii).

Embodiment 13. The precursor RNA polynucleotide of embodiment 12, wherein element (a)(i) is adjacent to element (a)(ii).

Embodiment 14. The precursor RNA polynucleotide of embodiment 2, wherein element (c)(ii) is located 3′ to element (c)(i).

Embodiment 15. The precursor RNA polynucleotide of embodiment 14, wherein element (c)(ii) is adjacent to element (c)(i).

Embodiment 16. The precursor RNA polynucleotide of any one of embodiments 6-9, wherein the spacer sequence is at least 5 nucleotides in length.

Embodiment 17. The precursor RNA polynucleotide of any one of embodiments 6-9, wherein the spacer comprises an unstructured, structured or randomly generated polynucleotide sequence.

Embodiment 18. The precursor RNA polynucleotide of embodiment 16 or 17, wherein the spacer is 5 to 60 nucleotides in length, inclusive.

Embodiment 19. The precursor RNA polynucleotide of any one of embodiments 1-18, wherein the 3′ and 5′ permuted intron segments each independently comprise a Group I intron segment, a Group II intron segment, a synthetic intron segment, or a variant thereof.

Embodiment 20. The precursor RNA polynucleotide of embodiment 19, wherein the 3′ permuted intron segment comprises a 3′ Group I intron segment or a variant thereof.

Embodiment 21. The precursor RNA polynucleotide of embodiment 19, wherein the 5′ permuted intron segment comprises a 5′ Group I intron segment or a variant thereof.

Embodiment 22. The precursor RNA polynucleotide of embodiment 19, wherein the 3′ permuted intron segment comprises a 3′ Group II intron segment or a variant thereof.

Embodiment 23. The precursor RNA polynucleotide of embodiment 19, wherein the 5′ permuted intron segment comprises a 5′ Group II intron segment or a variant thereof.

Embodiment 24. The precursor RNA polynucleotide of any one of embodiments 1-23, wherein element (a)(i) of the 5′ combined accessory element comprises a 5′ affinity tag, a 5′ external spacer, and the 3′ permuted intron segment.

Embodiment 25. The precursor RNA polynucleotide of any one of embodiments 1-24, wherein element (a)(ii) of the 5′ combined accessory element comprises the 3′ exon segment, a 5′ internal duplex sequence, and a 5′ internal spacer.

Embodiment 26. The precursor RNA polynucleotide of embodiment 24, wherein the 5′ affinity tag is adjacent to the 5′ external spacer.

Embodiment 27. The precursor RNA polynucleotide of embodiment 24-26, wherein the 5′ affinity tag is located 5′ to the 5′ external spacer.

Embodiment 28. The precursor RNA polynucleotide of embodiment 25, wherein the 5′ internal duplex sequence is adjacent to the 5′ internal spacer.

Embodiment 29. The precursor RNA polynucleotide of embodiment 25 or 28, wherein the 5′ internal duplex sequence is located 5′ to the 5′ internal spacer.

Embodiment 30. The precursor RNA polynucleotide of any one of embodiments 1-29, wherein element (c)(i) of the 3′ combined accessory element comprises a 3′ internal spacer, 3′ internal duplex sequence, and the 5′ exon segment.

Embodiment 31. The precursor RNA polynucleotide of any one of embodiments 1-30, wherein element (c)(ii) of the 3′ combined accessory element comprises the 5′ permuted intron segment, a 3′ external spacer, and a 3′ affinity tag.

Embodiment 32. The precursor RNA polynucleotide of embodiment 31, wherein the 3′ affinity tag is adjacent to the 3′ external spacer.

Embodiment 33. The precursor RNA polynucleotide of embodiment 31 or 32, wherein the 3′ affinity tag is located 3′ to the 3′ external spacer.

Embodiment 34. The precursor RNA polynucleotide of embodiment 30, wherein the 3′ internal duplex sequence is adjacent to the 3′ internal spacer.

Embodiment 35. The precursor RNA polynucleotide of embodiment 30 or 34, wherein the 3′ internal duplex sequence is located 3′ to the 3′ internal spacer.

Embodiment 36. The precursor RNA polynucleotide of any one of embodiments 1-35, wherein the element (a)(ii) of the 5′ combined accessory element comprises a 5′ internal duplex sequence located between the 3′ exon segment and the intervening region.

Embodiment 37. The precursor RNA polynucleotide of any one of embodiments 1-36, wherein element (c)(i) of the 3′ combined accessory element comprises a 3′ internal duplex sequence positioned between the intervening region and the 5′ exon segment.

Embodiment 38. The precursor RNA polynucleotide of any one of embodiments 1-37, wherein the polynucleotide comprises a 5′ internal duplex sequence and a 3′ internal duplex sequence.

Embodiment 39. The precursor RNA polynucleotide of any one of embodiments 1-38, comprising a polyA affinity tag.

Embodiment 40. The precursor RNA polynucleotide of any one of embodiments 1-39, wherein at least a portion of the exon segment is codon optimized.

Embodiment 41. The precursor RNA polynucleotide of any one of embodiments 1-40, further comprising a leading untranslated sequence at the 5′ end.

Embodiment 42. The precursor RNA polynucleotide of any one of embodiments 1-41, further comprising a lagging untranslated sequence at the 3′ end.

Embodiment 43. The precursor RNA polynucleotide of any one of embodiments 1-42, comprising a 5′ external spacer located between a leading untranslated sequence and the element (a)(i) of the 5′ combined accessory element.

Embodiment 44. The precursor RNA polynucleotide of any one of embodiments 1-43, comprising a 3′ external spacer located between element (c)(ii) of the 3′ combined accessory element and a lagging untranslated sequence.

Embodiment 45. The precursor RNA polynucleotide of any one of embodiments 1-44, further comprising an aptamer.

Embodiment 46. The precursor RNA polynucleotide of embodiment 45, wherein the aptamer is synthetic.

Embodiment 47. The precursor RNA polynucleotide of any one of embodiments 1-46, wherein the polynucleotide allows production of a circular RNA that is translatable or biologically active inside a eukaryotic cell.

Embodiment 48. The precursor RNA polynucleotide of any one of embodiments 1-47, wherein the intervening region comprises a coding element.

Embodiment 49. The precursor RNA polynucleotide of embodiment 48, wherein the coding element comprises a sequence encoding a therapeutic protein.

Embodiment 50. The precursor RNA polynucleotide of embodiment 48 or 49, wherein the intervening region further comprises a stop codon or stop cassette.

Embodiment 51. The precursor RNA polynucleotide of any one of embodiments 1-50, wherein the intervening region comprises one or more noncoding elements.

Embodiment 52. The precursor RNA polynucleotide of embodiment 51, wherein the noncoding element comprises an untranslated region (UTR).

Embodiment 53. The precursor RNA polynucleotide of embodiment 51, wherein the noncoding element is a natural 5′ UTR.

Embodiment 54. The precursor RNA polynucleotide of embodiment 51, wherein the noncoding element is a natural 3′ UTR.

Embodiment 55. The precursor RNA polynucleotide of embodiment 51, wherein the noncoding element is a synthetic spacer sequence.

Embodiment 56. The precursor RNA polynucleotide of embodiment 51, wherein the noncoding element is an aptamer.

Embodiment 57. The precursor RNA polynucleotide of embodiment 51, wherein the noncoding element is or comprises a translation initiation element (TIE).

Embodiment 58. The precursor RNA polynucleotide of embodiment 51, wherein the TIE comprises a viral or eukaryotic internal ribosome entry site (IRES) or a fragment or variant thereof.

Embodiment 59. The precursor RNA polynucleotide of embodiment 51, wherein the noncoding element comprises a lncRNA, miRNA, or a miRNA sponge.

Embodiment 60. The precursor RNA polynucleotide of any one of embodiments 1-59, wherein the 3′ permuted intron segment, 5′ permuted intron segment, or both the 3′ and 5′ permuted intron segments comprise a native Group I intron segment or Group II intron segment sequence.

Embodiment 61. The precursor RNA polynucleotide of any one of embodiments 1-60, wherein the 3′ permuted intron segment, 5′ permuted intron segment, or both the 3′ and 5′ permuted intron segments comprise one or more nucleotide substitutions of a native Group I intron segment or Group II intron segment sequence.

Embodiment 62. The precursor RNA polynucleotide of any one of embodiments 1-61, wherein the 3′ permuted intron segment, 5′ permuted intron segment, or both the 3′ and 5′ permuted intron segments comprise one or more nucleotide insertions of a native Group I intron segment or Group II intron segment sequence.

Embodiment 63. The precursor RNA polynucleotide of any one of embodiments 1-62, wherein the 3′ permuted intron segment, 5′ permuted intron segment, or both the 3′ and 5′ permuted intron segments comprise one or more nucleotide deletions of a native Group I intron segment or Group II intron segment sequence.

Embodiment 64. The precursor RNA polynucleotide of any one of embodiments 1-63, wherein the 3′ permuted intron segment, 5′ permuted intron segment, or both the 3′ and 5′ permuted intron segments comprise a nucleotide substitution of one or both the dinucleotide of a native Group I or Group II intron splice site dinucleotide.

Embodiment 65. The precursor RNA polynucleotide of any one of embodiments 1-64, wherein elements (a)(ii) and/or (c)(i) comprise a native Group I intron-adjacent exon segment sequence or Group II intron-adjacent exon segment sequence.

Embodiment 66. The precursor RNA polynucleotide of embodiment 65, wherein elements (a)(ii) and/or (c)(i) comprise at least one mutation of a native Group I intron-adjacent exon segment sequence or Group II intron-adjacent exon segment sequence.

Embodiment 67. The precursor RNA polynucleotide of embodiment 65, wherein elements (a)(ii) and/or (c)(i) comprise at least one deletion of a native Group I intron-adjacent exon segment sequence or Group II intron-adjacent exon segment sequence.

Embodiment 68. The precursor RNA polynucleotide of embodiment 65, wherein elements (a)(ii) and/or (c)(i) comprise at least one insertion of a native Group I intron-adjacent exon segment sequence or Group II intron-adjacent exon segment sequence.

Embodiment 69. The precursor RNA polynucleotide of any one of embodiments 1-68, further comprising a 5′ internal duplex sequence and a 3′ internal duplex sequence, wherein
(a) the 5′ internal duplex sequence is positioned between element (a)(ii) of the 5′ combined accessory element and the intervening region, and
(b) the 3′ internal duplex sequence is positioned between the intervening region and element (c)(i) of the 3′ combined accessory element.

Embodiment 70. The precursor RNA polynucleotide of embodiment 69, wherein the 5′ internal duplex sequence and 3′ internal duplex sequence are at least 80% complementary.

Embodiment 71. The precursor RNA polynucleotide of embodiment 69 or 70, wherein the 5′ internal duplex sequence and 3′ internal duplex sequence are each independently 5-20 nucleotides in length, inclusive.

Embodiment 72. The precursor RNA polynucleotide of any one of embodiments 69-71, wherein the 5′ and 3′ internal duplex sequences are predicted to form a contiguous duplex no longer than 35 nucleotides.

Embodiment 73. The precursor RNA polynucleotide of any one of embodiments 69-72, wherein the 5′ internal duplex sequence and 3′ internal duplex sequence each have a GC content of at least 10%.

Embodiment 74. The precursor RNA polynucleotide of any one of embodiments 1-73, wherein at least one of the exon segments are less than 15 nucleotides in length.

Embodiment 75. The precursor RNA polynucleotide of any one of embodiments 1-74, wherein the 3′ exon segment and/or 5′ exon segment comprises a Group I exon segment or a Group II exon segment less than 15 nucleotides in length.

Embodiment 76. The precursor RNA polynucleotide of any one of embodiments 1-75, wherein the 3′ permuted intron segment, 5′ permuted intron segment, or both the 3′ and 5′ permuted intron segments are at least 50 nucleotides in length.

Embodiment 77. The precursor RNA polynucleotide of embodiment 76, wherein the 3′ permuted intron segment, 5′ permuted intron segment, or both the 3′ and 5′ permuted intron segments have a percent sequence identity of about 70%, 75%, 80%, 85%, 90%, 91%, 92%, 93%, 94%, 95%, 96%, 97%, 98%, 99%, or more to a naturally occurring intron.

Embodiment 78. The precursor RNA polynucleotide of any one of embodiments 1-77, wherein the 3′ permuted intron segment comprises a 3′ Group I or Group II intron segment derived from a gene selected from a genus and/or species selected from Tables A or B and/or the 5′ permuted intron segment comprises a 5′ Group I or Group II intron segment derived from a gene selected from a genus and/or species selected from Tables A or B.

Embodiment 79. The precursor RNA polynucleotide of embodiment 78, wherein the 3′ Group I or Group II intron segment or the 5′ Group I or Group II intron segment are derived from a gene from a species selected from: Cyanobacterium Anabaena sp., T4 phage, Hypocrea pallida, Bulbithecium hyalosporum, Myoarachis inversa, Geosmithia argillacea, Coxiella burnetii, Agrobacterium tumefaciens, Azoarcus, Nostoc, Cordyceps capitata, Prochlorothrix hollandica, Tilletiopsis orzyzicola, Tetrahymena thermophila, and Staphylococcus phage Twort.

Embodiment 80. The precursor RNA polynucleotide of embodiment 78 or 79, wherein the 3′ Group I or Group II intron segment or the 5′ Group I or Group II intron segment comprises one, two, three, four, five, six, seven, eight, nine, ten, or more modifications of a native Group I intron or Group II intron sequence.

Embodiment 81. The precursor RNA polynucleotide of embodiment 80, wherein the modifications are mutations selected from the group consisting of: insertion, deletion, mutation, addition, and subtraction.

Embodiment 82. The precursor RNA polynucleotide of embodiment 80 or 81 wherein the modifications are deletions of two or more nucleotides of the 3′ Group I or Group II intron segment or the 5′ Group I or Group II intron segment, or combinations thereof.

Embodiment 83. The precursor RNA polynucleotide of embodiment 82, wherein the modifications are two or more deletions of the 5′ Group I intron segment at the 3′ end.

Embodiment 84. The precursor RNA polynucleotide of embodiment 82 or 83, wherein the modifications are two or more deletions of the 3′ Group I intron segment at the 5′ end.

Embodiment 85. The precursor RNA polynucleotide of any one of embodiments 76-84, wherein the 3′ and/or 5′ permuted intron segment comprise a polynucleotide sequence having a percent sequence identity of about 70%, 75%, 80%, 85%, 90%, 91%, 92%, 93%, 94%, 95%, 96%, 97%, 98%, 99%, or more to a naturally occurring intron a sequence set forth in SEQ ID NO: 2990-3187.

Embodiment 86. The precursor RNA polynucleotide of embodiment 85, wherein the 3′ and/or 5′ permuted intron segment comprise a polynucleotide sequence selected from a sequence set forth in SEQ ID NO: 2990-3668.

Embodiment 87. The precursor RNA polynucleotide of any one of embodiments 76-85, further comprising a translation initiation element (TIE).

Embodiment 88. The precursor RNA polynucleotide of embodiment 87, comprising an internal ribosome entry site (IRES), wherein the IRES comprises a viral IRES or eukaryotic IRES.

Embodiment 89. A polynucleotide encoding the precursor RNA polynucleotide of any one of embodiments 1-88.

Embodiment 90. The polynucleotide of embodiment 89, wherein the polynucleotide is an expression vector; optionally wherein the expression vector comprises an RNA polymerase promoter sequence.

Embodiment 91. The polynucleotide of embodiment 89, wherein the polynucleotide is selected from a DNA plasmid, a cosmid, a PCR product, dbDNA, close-ended DNA (ceDNA), and a viral polynucleotide.

Embodiment 92. The polynucleotide of any one of embodiments 89-91, further comprising a promoter segment.

Embodiment 93. The polynucleotide of embodiment 92, wherein the promoter segment comprises T7 promoter, SP6 promoter, or a fragment thereof.

Embodiment 94. A circular RNA polynucleotide produced by the precursor RNA polynucleotide of any one of embodiments 1-88 or the polynucleotide of any one of embodiments 89-93.

Embodiment 95. A circular RNA polynucleotide comprising the following elements operably connected to each other and arranged in the following sequence:
(a) a 5′ combined accessory element comprising a 3′ exon segment comprising a Group I or Group II exon 3′ nucleotide of a 3′ splice site dinucleotide;
(b) an intervening region; and
(c) a 3′ combined accessory element comprising a 5′ exon segment comprising a Group I or Group II exon 5′ nucleotide of a 5′ splice site dinucleotide.

Embodiment 96. The circular RNA polynucleotide of embodiment 95, wherein the 5′ combined accessory element comprises the second nucleotide of a 3′ Group I or Group II exon splice site dinucleotide and a natural exon sequence.

Embodiment 97. The circular RNA polynucleotide of embodiment 95 or 96, wherein the 3′ combined accessory element fragment comprises the first nucleotide of a 5′ Group I or Group II splice site dinucleotide and a natural exon sequence.

Embodiment 98. The circular RNA polynucleotide of any one of embodiments 95-97, wherein the 5′ combined accessory element comprises a 5′ internal duplex; and wherein the 3′ combined accessory element comprises a 3′ internal duplex.

Embodiment 99. The circular RNA polynucleotide of any one of embodiments 95-98, wherein the 5′ combined accessory element comprises a 5′ internal spacer.

Embodiment 100. The circular RNA polynucleotide of any one of embodiments 95-99, wherein the 3′ combined accessory element comprises a 3′ internal spacer.

Embodiment 101. The circular RNA polynucleotide of any one of embodiments 95-100, wherein the intervening region comprises a noncoding sequence.

Embodiment 102. The circular RNA polynucleotide of any one of embodiments 95-101, wherein the circular RNA polynucleotide is from about 50 nucleotides to about 15 kilobases in length.

Embodiment 103. The circular RNA polynucleotide of any one of embodiments 95-102, having an in vivo duration of therapeutic effect in a subject of at least about 10 hours.

Embodiment 104. The circular RNA polynucleotide of any one of embodiments 95-103, having a functional half-life of at least about 10 hours.

Embodiment 105. The circular RNA polynucleotide of any one of embodiments 95-104, having a duration of therapeutic effect in a cell greater than or equal to that of an equivalent linear RNA polynucleotide comprising the same expression sequence.

Embodiment 106. The circular RNA polynucleotide of any one of embodiments 95-105, having a functional half-life in a cell greater than or equal to that of an equivalent linear RNA polynucleotide comprising the same expression sequence.

Embodiment 107. The circular RNA polynucleotide of any one of embodiments 95-106, having an in vivo duration of therapeutic effect in a subject greater than that of an equivalent linear RNA polynucleotide having the same expression sequence.

Embodiment 108. The circular RNA polynucleotide of any one of embodiments 95-107, having an in vivo functional half-life in a subject greater than that of an equivalent linear RNA polynucleotide having the same expression sequence.

Embodiment 109. A cell comprising the precursor RNA polynucleotide of any one of embodiments 1-88, the polynucleotide of any one of embodiments 89-93, the circular RNA polynucleotide of any one of embodiments 94-108, or combinations thereof.

Embodiment 110. A lipid nanoparticle comprising the precursor RNA polynucleotide of any one of embodiments 1-88, the polynucleotide of any one of embodiments 89-93, the circular RNA polynucleotide of any one of embodiments 94-108, or combinations thereof.

Embodiment 111. A pharmaceutical composition comprising the precursor RNA polynucleotide of any one of embodiments 1-88, the polynucleotide of any one of embodiments 89-93, the circular RNA polynucleotide of any one of embodiments 94-108, or combinations thereof; and a transfer vehicle.

Embodiment 112. The pharmaceutical composition of embodiment 111, wherein the transfer vehicle comprises at least one of (i) an ionizable lipid, (ii) a structural lipid, and (iii) a PEG-modified lipid.

Embodiment 113. The pharmaceutical composition of embodiment 111 or 112, further comprising a targeting moiety.

Embodiment 114. The pharmaceutical composition of embodiment 113, wherein the targeting moiety mediates receptor-mediated endocytosis or direct fusion into selected cells of a selected cell population or tissue in the absence of cell isolation or purification.

Embodiment 115. A method of producing circularized RNA, comprising transcribing a DNA polynucleotide sequence that is complementary to a precursor RNA polynucleotide of any one of embodiments 1-88.

Embodiment 116. The method of embodiment 115, wherein the transcribing is performed in vitro.

Embodiment 117. The method of embodiment 115, wherein the transcribing is performed in a cell.

Embodiment 118. The method of embodiment 116, wherein the transcribing is performed in a cell selected from an immune cell, muscle cell, neural cell, epithelial cell and a tumor cell.

Embodiment 119. A method of producing a protein of interest by introducing the pharmaceutical composition of any one of embodiments 111-114.

Embodiment 120. A method of treating a subject in need thereof, comprising administering to the subject the pharmaceutical composition of any one of embodiments 111-114.

Embodiment 121. The method of embodiment 120, wherein the subject has a disease or disorder whereby expression of an RNA or protein encoded by elements (a)(ii) and/or (c)(i) alleviates one or more symptoms of the disease or disorder in the subject.

Embodiment 122. A method of identifying a combined accessory element comprising a modified Group I or Group II exon and/or intron segment that allows production of a circular RNA that is translatable or biologically active inside a eukaryotic cell, comprising:
(i) inserting 5′ and 3′ Group I or Group II intronic sequences derived from a database of native intronic sequence into a precursor RNA polynucleotide of any one of embodiments 1-88;
(ii) transcribing the polynucleotide into RNA in vitro or allowing the polynucleotide to be transcribed into RNA by a cell; and
(iii) determining the circularization efficiency of the RNA produced by the polynucleotide by identifying the amount of circularized RNA, the amount of excised intronic sequences, the amount of precursor RNA remaining after circularization, and combinations thereof.

Embodiment 123. The method of embodiment 122, wherein the modified Group I or Group II exon and/or intron segment comprises at least one modification selected from a deletion, insertion or substitution of at least one nucleotide.

Embodiment 124. The method of embodiment 122, wherein the at least one modification of a native Group I intron or Group II intron segment is a nucleotide substitution of one or both the dinucleotides of the 5′ and/or 3′ Group I intron splice site dinucleotides.

Embodiment 125. The method of any one of embodiments 122-124, wherein the 5′ or 3′ Group I or Group II intronic sequences, or combinations thereof are sequenced.

Embodiment 126. A method of determining a polynucleotide sequence that improves RNA circularization efficiency compared to a polynucleotide comprising a native intronic sequence or to a parent polynucleotide with a known sequence, the method comprising modifying a DNA sequence encoding the precursor RNA polynucleotide of any one of embodiments 1-88, the modifying comprising:
(i) modifying at least one nucleotide and/or altering the length of the element (a)(i) of the 5′ combined accessory element and/or element (c)(ii) of the 3′ combined accessory element of the DNA sequence encoding the precursor RNA polynucleotide of any one of embodiments 1-88;
(ii) altering the length of the 5′ and/or 3′ internal and/or external spacer sequence of the DNA sequence encoding precursor RNA polynucleotide any one of embodiments 1-88;
(iii) altering the length of the 5′ and/or 3′ internal duplex sequence of the DNA sequence encoding the precursor RNA polynucleotide any one of embodiments 1-88;
(iv) altering the length of the 5′ and/or 3′ exon sequence of the DNA sequence encoding the precursor RNA polynucleotide of any one of embodiments 1-88; or
(v) combinations thereof, and
transcribing the polynucleotide comprising the DNA sequence into RNA in vitro or allowing the polynucleotide comprising the DNA sequence to be transcribed into RNA by a cell; and determining the circularization efficiency of the RNA produced by the polynucleotide comprising the DNA sequence by identifying the amount of circularized RNA, the amount of excised intronic sequences, the amount of precursor RNA remaining after circularization, and combinations thereof.

Embodiment 127. The method of any one of embodiments 122-126, further comprising comparing the circularization efficiency of the polynucleotide with a polynucleotide comprising a native intronic sequence, or a parent polynucleotide.

Embodiment 128. A precursor RNA polynucleotide comprising:
a) a terminal element upstream of an intervening region and a monotron element, wherein
   i) the terminal element comprises a splice site nucleotide,
   ii) the monotron element comprises a splice site dinucleotide at or near the 5′ end of the monotron, and
   iii) the monotron element is capable of interacting with a nucleophile that is capable of cleaving at the splice site dinucleotide at or near the 5′ end of the monotron, wherein
the cleavage product of iii) comprises a 5′ splice site nucleotide that is capable of cleaving at the splice site nucleotide of the terminal element; or
b) a monotron element upstream of an intervening region and a terminal element, wherein
   i) the monotron element comprises a splice site dinucleotide at or near the 3′ end of the monotron,
   ii) the terminal element comprises a splice site nucleotide, and
   iii) the monotron element is capable of interacting with a nucleophile that is capable of cleaving at the splice site nucleotide of the terminal element, wherein the cleavage product of iii) comprises a 5′ splice site nucleotide that is capable of cleaving at the splice site dinucleotide at or near the 3′ end of the monotron.

Embodiment 129. The precursor RNA polynucleotide of embodiment 128, wherein the terminal element comprises a terminal segment that is retained upon circularization.

Embodiment 130. The precursor RNA polynucleotide of embodiment 128 or 129, wherein the splice site nucleotide of the terminal element is not a natural splice site dinucleotide associated with a natural Group I or Group II intron sequence.

Embodiment 131. The precursor RNA polynucleotide of any one of embodiments 128-130, wherein the terminal element is not excised upon cleavage and is retained post-cleavage.

Embodiment 132. The precursor RNA polynucleotide of any one of embodiments 128-131, wherein the terminal element comprises a natural exon or fragment thereof.

Embodiment 133. The precursor RNA polynucleotide of embodiment 132, wherein the natural exon or fragment thereof is 10-20 nucleotides in length.

Embodiment 134. The precursor RNA polynucleotide of embodiment 132 or 133, wherein the natural exon is a Group I or Group II exon.

Embodiment 135. The precursor RNA polynucleotide of any one of embodiments 132-134, wherein terminal element comprises a synthetic derivative of a natural exon or fragment thereof.

Embodiment 136. The precursor RNA polynucleotide of any one of embodiments 128-135, wherein the terminal element is capable of directing or functionalizing the splicing activity of the monotron element.

Embodiment 137. The precursor RNA polynucleotide of any one of embodiments 128-136, wherein the monotron element comprises at least a portion of a Group I or Group II intron and wherein the at least a portion is at least 10 nucleotides in length.

Embodiment 138. The precursor RNA polynucleotide of any one of embodiments 128-137, wherein the monotron element is capable of inducing circularization when it interacts with the terminal element.

Embodiment 139. The precursor RNA polynucleotide of embodiment 128, wherein all or a portion of the terminal element is excised post-circularization.

Embodiment 140. The precursor RNA polynucleotide of embodiment 128, wherein all or a portion of the monotron element is excised post-circularization.

Embodiment 141. The precursor RNA polynucleotide of embodiment 128, wherein the monotron element is fully excised post-circularization.

Embodiment 142. The precursor RNA polynucleotide of any one of embodiments 128-141, wherein the monotron element is derived from a Group I or Group II intron from a gene selected from: Cyanobacterium Anabaena sp., T4 phage, Hypocrea pallida, Bulbithecium hyalosporum, Myoarachis inversa, Geosmithia argillacea, Coxiella burnetii, Agrobacterium tumefaciens, Azoarcus, Nostoc, Cordyceps capitata, Prochlorothrix hollandica, and Tilletiopsis orzyzicola.

Embodiment 143. The precursor RNA polynucleotide of any one of embodiments 128-142 wherein the terminal element is less than 500 nucleotides in length.

Embodiment 144. The precursor RNA polynucleotide of any one of embodiments 128-143 wherein the monotron element is less than 500 nucleotides in length.

Embodiment 145. The precursor RNA polynucleotide of any one of embodiments 128-144, wherein (i) the nucleophile is a guanosine; and/or (ii) the 5′ splice site nucleotide of the cleavage product has a 3′ hydroxyl group.

Embodiment 146. The precursor RNA polynucleotide of any one of embodiments 128-145, further comprising at least one affinity tag; optionally wherein the affinity tag is a polyA affinity tag.

Embodiment 147. The precursor RNA polynucleotide of any one of embodiments 128-146, wherein the terminal element comprises (a) a 5′ affinity tag; or (b) a 3′ affinity tag.

Embodiment 148. The precursor RNA polynucleotide of any one of embodiments 128-147, wherein the monotron element comprises (a) a 3′ affinity tag; or (b) a 5′ affinity tag.

Embodiment 149. The precursor RNA polynucleotide of any one of embodiments 128-148, further comprising an internal spacer sequence positioned between the terminal element and the intervening region.

Embodiment 150. The precursor RNA polynucleotide of any one of embodiments 128-149, further comprising an internal spacer sequence positioned between the intervening region and the monotron element.

Embodiment 151. The precursor RNA polynucleotide of any one of embodiments 128-150, further comprising an external spacer positioned adjacent to the terminal element.

Embodiment 152. The precursor RNA polynucleotide of any one of embodiments 128-151, further comprising an external spacer positioned adjacent to the monotron element.

Embodiment 153. The precursor RNA polynucleotide of any one of embodiments 149-152, wherein one or more of the internal spacers, or external spacers, comprise an unstructured, structured or randomly generated polynucleotide sequence.

Embodiment 154. The precursor RNA polynucleotide of embodiment 153, wherein one or more of the internal spacers, or external spacers, is 5-60 nucleotides in length, inclusive.

Embodiment 155. The precursor RNA polynucleotide of any one of embodiments 128-154, further comprising a 5′ internal duplex sequence and a 3′ internal duplex sequence; wherein
(a) if the terminal element is upstream of the monotron element, the 5′ internal duplex sequence is positioned between the terminal element and the intervening region, and the 3′ internal duplex sequence is positioned between the intervening region and the monotron element; or
(b) if the monotron element is upstream of the terminal element, the 5′ internal duplex sequence is positioned between monotron and the intervening region, and the 3′ internal duplex sequence is positioned between the intervening region and the terminal element.

Embodiment 156. The precursor RNA polynucleotide of embodiment 155, wherein the 5′ internal duplex sequence and 3′ internal duplex sequence are at least 80% complementary.

Embodiment 157. The precursor RNA polynucleotide of embodiment 155 or 156, wherein the 5′ internal duplex sequence and/or 3′ internal duplex sequence are 5-20 nucleotides in length, inclusive.

Embodiment 158. The precursor RNA polynucleotide of any one of embodiments 155-157, wherein the 5′ and 3′ internal duplex sequences are predicted to form a contiguous duplex no longer than 35 nucleotides.

Embodiment 159. The precursor RNA polynucleotide of any one of embodiments 155-158, wherein the 5′ internal duplex sequence and/or 3′ internal duplex sequence each have a GC content of at least 10%.

Embodiment 160. The precursor RNA polynucleotide of embodiment 147, wherein the 5′ affinity tag is positioned adjacent to the 5′ external spacer.

Embodiment 161. The precursor RNA polynucleotide of embodiment 160, wherein the 5′ affinity tag is positioned 5′ to the 5′ external spacer.

Embodiment 162. The precursor RNA polynucleotide of embodiment 148, wherein the 3′ affinity tag is positioned adjacent to the 3′ external spacer.

Embodiment 163. The precursor RNA polynucleotide of embodiment 162, wherein the 3′ affinity tag is positioned 3′ to the 3′ external spacer.

Embodiment 164. The precursor RNA polynucleotide of embodiment 155, wherein the 5′ internal duplex is positioned adjacent to the 5′ internal spacer, optionally wherein the 5′ internal duplex is positioned 5′ to the 5′ internal spacer.

Embodiment 165. The precursor RNA polynucleotide of embodiment 155, wherein the 3′ internal duplex is positioned adjacent to the 3′ internal spacer, optionally wherein the 3′ internal duplex is positioned 3′ to the 3′ internal spacer.

Embodiment 166. The precursor RNA polynucleotide of any one of embodiments 128-165, comprising a 3′ and/or 5′ exon segment, wherein at least a portion of the 3′ and/or 5′ exon segment is codon optimized.

Embodiment 167. The precursor RNA polynucleotide of any one of embodiments 128-166, further comprising a leading untranslated sequence.

Embodiment 168. The precursor RNA polynucleotide of any one of embodiments 128-167, further comprising a lagging untranslated sequence.

Embodiment 169. The precursor RNA polynucleotide of any one of embodiments 128-168, comprising a 5′ external spacer positioned between a leading untranslated sequence and (a) if the terminal element is upstream of the monotron element, the terminal element or (b) if the monotron element is upstream of the terminal element, the monotron element.

Embodiment 170. The precursor RNA polynucleotide of any one of embodiments 128-169, comprising a 3′ external spacer positioned between (a) if the terminal element is upstream of the monotron element, the monotron element and a lagging untranslated sequence; or (b) if the monotron element is upstream of the terminal element, the terminal element and a lagging untranslated sequence.

Embodiment 171. The precursor RNA polynucleotide of any one of embodiments 128-170, further comprising an aptamer.

Embodiment 172. The precursor RNA polynucleotide of embodiment 171, wherein the aptamer is synthetic.

Embodiment 173. The precursor RNA polynucleotide of any one of embodiments 128-172, wherein the polynucleotide allows production of a circular RNA that is translatable or biologically active inside a eukaryotic cell.

Embodiment 174. The precursor RNA polynucleotide of any one of embodiments 128-173, wherein the intervening region comprises a coding element.

Embodiment 175. The precursor RNA polynucleotide of embodiment 174, wherein the coding element comprises a sequence encoding a therapeutic protein.

Embodiment 176. The precursor RNA polynucleotide of embodiment 174 or 175, further comprising a stop codon or stop cassette.

Embodiment 177. The precursor RNA polynucleotide of any one of embodiments 128-176, wherein the intervening region comprises an untranslated region.

Embodiment 178. The precursor RNA polynucleotide of embodiment 177, wherein the untranslated region comprises one or more noncoding elements.

Embodiment 179. The precursor RNA polynucleotide of embodiment 178, wherein the noncoding element is a natural 5′ Untranslated Region (UTR).

Embodiment 180. The precursor RNA polynucleotide of embodiment 178, wherein the noncoding element is a natural 3′ Untranslated Region (UTR).

Embodiment 181. The precursor RNA polynucleotide of embodiment 178, wherein the noncoding element is a synthetic spacer sequence.

Embodiment 182. The precursor RNA polynucleotide of embodiment 178, wherein the noncoding element is an aptamer.

Embodiment 183. The precursor RNA polynucleotide of embodiment 178, wherein the noncoding element is or comprises a Translation Initiation Element (TIE).

Embodiment 184. The precursor RNA polynucleotide of embodiment 183, wherein the TIE comprises a viral or eukaryotic internal ribosome entry site (IRES).

Embodiment 185. The precursor RNA polynucleotide of embodiment 178, wherein the noncoding element comprises a lncRNA, miRNA, or a miRNA sponge.

Embodiment 186. The precursor RNA polynucleotide of any one of embodiments 128-185, wherein the polynucleotide comprises at least one mutation of a native Group I intron-adjacent exon sequence or Group II intron-adjacent exon sequence.

Embodiment 187. The precursor RNA polynucleotide of embodiment 186, wherein the polynucleotide comprises at least one substation mutation of a native Group I intron-adjacent exon sequence or Group II intron-adjacent exon sequence.

Embodiment 188. The precursor RNA polynucleotide of embodiment 186, wherein the polynucleotide comprises at least one deletion of a native Group I intron-adjacent exon sequence or Group II intron-adjacent exon sequence.

Embodiment 189. The precursor RNA polynucleotide of embodiment 186, wherein the polynucleotide comprises at least one insertion of a native Group I intron-adjacent exon sequence or Group II intron-adjacent exon sequence.

Embodiment 190. The precursor RNA polynucleotide of any one of embodiments 128-189, wherein the polynucleotide comprises at least one exon segment less than 15 nucleotides in length.

Embodiment 191. The precursor RNA polynucleotide of any one of embodiments 128-190, wherein the polynucleotide comprises a 3′ exon segment and/or 5′ exon segment, wherein the 3′ or 5′ exon segment comprises a Group I exon segment or a Group II exon segment less than 15 nucleotides in length.

Embodiment 192. The precursor RNA polynucleotide of any one of embodiment 191, wherein the 3′ exon segments and/or 5′ exon segments, have a percent sequence identity of about 70%, 75%, 80%, 85%, 90%, 91%, 92%, 93%, 94%, 95%, 96%, 97%, 98%, 99% or more to a native Group I intron-adjacent exon sequence or Group II intron-adjacent exon sequence.

Embodiment 193. The precursor RNA polynucleotide of any one of embodiments 128-192, wherein the terminal element sequence has a percent sequence identity of about 70%, 75%, 80%, 85%, 90%, 91%, 92%, 93%, 94%, 95%, 96%, 97%, 98%, 99% or more to an exon fragment of a sequence selected from Tables A or B.

Embodiment 194. The precursor RNA polynucleotide of any one of embodiments 128-193, wherein the terminal element sequence comprises an exon fragment comprising one, two, three, four, five, six, seven, eight, nine, ten, or more modifications to a sequence selected from Tables A or B.

Embodiment 195. The precursor RNA polynucleotide of embodiment 194, wherein the modifications are mutations selected from the group consisting of: insertion, deletion, mutation, addition, and subtraction.

Embodiment 196. The precursor RNA polynucleotide of any one of embodiments 128-194, wherein the terminal element or exon fragment thereof comprises a polynucleotide sequence selected from a sequence set forth in SEQ ID NO: 2990-3668.

Embodiment 197. The precursor RNA polynucleotide of any one of embodiments 128-196, wherein the monotron element sequence or fragment thereof has a percent sequence identity of about 70%, 75%, 80%, 85%, 90%, 91%, 92%, 93%, 94%, 95%, 96%, 97%, 98%, 99% or more to a sequence selected from Tables A or B.

Embodiment 198. The precursor RNA polynucleotide of any one of embodiments 128-197, wherein the monotron element sequence or fragment thereof comprises one, two, three, four, five, six, seven, eight, nine, ten, or more modifications to a sequence selected from Tables A or B.

Embodiment 199. The precursor RNA polynucleotide of embodiment 198, wherein the modifications are mutations selected from the group consisting of: insertion, deletion, mutation, addition, and subtraction.

Embodiment 200. The precursor RNA polynucleotide of any one of embodiments 128-199, wherein the monotron element or fragment thereof comprises a polynucleotide sequence selected from a sequence set forth in SEQ ID NO: 2990-3668.

Embodiment 201. A polynucleotide encoding the precursor RNA polynucleotide of any one of embodiments 128-200.

Embodiment 202. The polynucleotide of embodiment 201, wherein the polynucleotide is an expression vector; optionally wherein the expression vector comprises an RNA polymerase promoter sequence.

Embodiment 203. The polynucleotide of embodiment 202, wherein the polynucleotide is selected from a DNA plasmid, a cosmid, a PCR product, dbDNA close-ended DNA (ceDNA), and a viral polynucleotide.

Embodiment 204. The polynucleotide of any one of embodiments 201-203, further comprising a promoter segment.

Embodiment 205. The polynucleotide of embodiment 204, wherein the promoter segment comprises T7 promoter, SP6 promoter or a fragment thereof.

Embodiment 206. A circular RNA polynucleotide produced by the precursor RNA polynucleotide of any one of embodiments 128-200 or the polynucleotide of any one of embodiments 201-205.

Embodiment 207. A circular RNA polynucleotide comprising the following elements operably connected to each other and arranged in the following sequence:
(a) at least a portion of a terminal element,
(b) a 3′ exon segment comprising a 3′ nucleotide of a 3′ splice site dinucleotide,
(c) an intervening region,
(d) a 5′ exon segment comprising a 5′ nucleotide of a 5′ splice site dinucleotide, and
(e) at least a portion of a monotron element;
wherein the 5′ and/or 3′ splice site dinucleotides are distinct from the natural splice site dinucleotide(s) associated with a natural Group I or Group II intron sequence.

Embodiment 208. The circular RNA polynucleotide of embodiment 207, wherein element (b) comprises the second nucleotide of a 3′ Group I or Group II exon splice site dinucleotide and a natural exon sequence.

Embodiment 209. The circular RNA polynucleotide of embodiment 207 or 208, wherein element (d) comprises the first nucleotide of a 5′ Group I or Group II splice site dinucleotide and a natural exon sequence.

Embodiment 210. The circular RNA polynucleotide of any one of embodiments 207-209, comprising a 5′ internal duplex and a 3′ internal duplex.

Embodiment 211. The circular RNA polynucleotide of any one of embodiments 207-210, comprising a 5′ internal spacer.

Embodiment 212. The circular RNA polynucleotide of any one of embodiments 207-211, comprising a 3′ internal spacer.

Embodiment 213. The circular RNA polynucleotide of any one of embodiments 207-212, wherein the intervening region a.

Embodiment 214. The circular RNA polynucleotide of any one of embodiments 207-213, wherein the circular RNA polynucleotide is from about 50 nucleotides to about 15 kilobases in length.

Embodiment 215. The circular RNA polynucleotide of any one of embodiments 207-214, having an in vivo duration of therapeutic effect in a subject of at least about 10 hours.

Embodiment 216. The circular RNA polynucleotide of any one of embodiments 207-215, having a functional half-life of at least about 10 hours.

Embodiment 217. The circular RNA polynucleotide of any one of embodiments 207-216, having a duration of therapeutic effect in a cell greater than or equal to that of an equivalent linear RNA polynucleotide comprising the same expression sequence.

Embodiment 218. The circular RNA polynucleotide of any one of embodiments 207-217, having a functional half-life in a cell greater than or equal to that of an equivalent linear RNA polynucleotide comprising the same expression sequence.

Embodiment 219. The circular RNA polynucleotide of any one of embodiments 207-218, having an in vivo duration of therapeutic effect in a subject greater than that of an equivalent linear RNA polynucleotide having the same expression sequence.

Embodiment 220. The circular RNA polynucleotide of any one of embodiments 207-219, having an in vivo functional half-life in a subject greater than that of an equivalent linear RNA polynucleotide having the same expression sequence.

Embodiment 221. A cell comprising the precursor RNA polynucleotide of any one of embodiments 128-200, the polynucleotide of any one of embodiments 201-205, the circular RNA polynucleotide of any one of embodiments 206-220, or combinations thereof.

Embodiment 222. A lipid nanoparticle comprising the precursor RNA polynucleotide of any one of embodiments 128-200, the polynucleotide of any one of embodiments 201-205, the circular RNA polynucleotide of any one of embodiments 206-220, or combinations thereof.

Embodiment 223. A pharmaceutical composition comprising the circular RNA polynucleotide of any one of embodiments 206-220 and a transfer vehicle.

Embodiment 224. The pharmaceutical composition of embodiment 223, wherein the transfer vehicle comprises at least one of (i) an ionizable lipid, (ii) a structural lipid, and (iii) a PEG-modified lipid.

Embodiment 225. The pharmaceutical composition of embodiment 223 or 224, further comprising a targeting moiety.

Embodiment 226. The pharmaceutical composition of embodiment 225, wherein the targeting moiety mediates receptor-mediated endocytosis or direct fusion into selected cells of a selected cell population or tissue in the absence of cell isolation or purification.

Embodiment 227. A method of producing circularized RNA, comprising transcribing a DNA polynucleotide sequence that is complementary to a precursor RNA polynucleotide of any one of embodiments 128-200.

Embodiment 228. The method of embodiment 227, wherein the transcribing is performed in vitro.

Embodiment 229. The method of embodiment 228, wherein the transcribing is performed in a cell.

Embodiment 230. The method of embodiment 229, wherein the transcribing is performed in a cell selected from an immune cell, muscle cell, neural cell, epithelial cell and a tumor cell.

Embodiment 231. A method of producing a protein of interest by introducing the pharmaceutical composition of any one of embodiments 223-226.

Embodiment 232. A method of treating a subject in need thereof, comprising administering to the subject the pharmaceutical composition of any one of embodiments 223-226 or the lipid nanoparticle of embodiment 222.

Embodiment 233. The method of embodiment 232, wherein the subject has a disease or disorder whereby expression of an RNA or protein encoded by exon elements alleviates one or more symptoms of the disease or disorder in the subject.

Embodiment 234. A method of identifying an monotron element and terminal element pair that allows production of a circular RNA that is translatable or biologically active inside a eukaryotic cell, comprising:
inserting a modified 5′ and 3′ Group I or Group II intron sequence derived from a database of native intronic sequence to form a monotron element into a precursor RNA polynucleotide of any one of embodiments 128-200;
inserting a synthetic polynucleotide sequence to form a terminal element into a precursor RNA polynucleotide of any one of embodiments 128-200;
transcribing the polynucleotide into RNA in vitro or allowing the polynucleotide to be transcribed into RNA by a cell; and
determining the circularization efficiency of the RNA produced by the polynucleotide by identifying the amount of circularized RNA, the amount of excised intronic sequences, the amount of precursor RNA remaining after circularization, and combinations thereof.

Embodiment 235. The method of embodiment 234, wherein the modified 5′ and 3′ Group I or Group II intron sequence comprises at least one modification selected from a deletion, insertion or substitution of at least one nucleotide.

Embodiment 236. The method of any one of embodiments 234 and 235, wherein the 5′ or 3′ Group I or Group II intronic sequences, or combinations thereof are sequenced.

Embodiment 237. A method of determining a polynucleotide sequence that improves RNA circularization efficiency compared to a polynucleotide comprising a native intronic sequence or to a parent polynucleotide with a known sequence, the method comprising modifying a DNA sequence encoding the precursor RNA polynucleotide of any one of embodiments 128-200, the modifying comprising:
(i) modifying at least one nucleotide and/or altering the length of the terminal element and/or monotron element of the DNA sequence encoding the precursor RNA polynucleotide of any one of embodiments 128-200;
(ii) altering the length of the 5′ and/or 3′ internal and/or external spacer sequence of the DNA sequence encoding precursor RNA polynucleotide any one of embodiments 128-200;
(iii) altering the length of the 5′ and/or 3′ internal duplex sequence of the DNA sequence encoding the precursor RNA polynucleotide any one of embodiments 128-200;
(iv) altering the length of the 5′ and/or 3′ exon sequence of the DNA sequence encoding the precursor RNA polynucleotide of any one of embodiments 128-200;
(iv) or combinations thereof, and
transcribing the polynucleotide comprising the DNA sequence into RNA in vitro or allowing the polynucleotide comprising the DNA sequence to be transcribed into RNA by a cell; and determining the circularization efficiency of the RNA produced by the polynucleotide comprising the DNA sequence by identifying the amount of circularized RNA, the amount of excised intronic sequences, the amount of precursor RNA remaining after circularization, and combinations thereof.

Embodiment 238. The method of any one of embodiments 234-237, further comprising comparing the circularization efficiency of the polynucleotide with a polynucleotide comprising a native intronic sequence, or a parent polynucleotide.

Embodiment 239. The precursor RNA polynucleotide of any one of embodiments 128-200, wherein the terminal element is upstream of the monotron element.

Embodiment 240. The precursor RNA polynucleotide of any one of embodiments 128-200, wherein the monotron element is upstream of the terminal element.

Embodiment 241. The polynucleotide of any one of embodiments 201-204, wherein the polynucleotide encodes a precursor RNA polynucleotide comprising the terminal element upstream of the monotron element.

Embodiment 242. The polynucleotide of any one of embodiments 201-204, wherein the polynucleotide encodes a precursor RNA polynucleotide comprising the monotron element upstream of the terminal element.

Embodiment 243. A circular RNA polynucleotide produced by the precursor RNA polynucleotide of any one of embodiments 239-240 or the polynucleotide of any one of embodiments 241-242.

Embodiment 244. A cell comprising the precursor RNA polynucleotide of any one of embodiments 239-240, the polynucleotide of any one of embodiments 241-242, or the circular RNA polynucleotide of embodiment 243.

Embodiment 245. A lipid nanoparticle comprising the precursor RNA polynucleotide of any one of embodiments 239-240, the polynucleotide of any one of embodiments 241-242, or the circular RNA polynucleotide of embodiment 243, or combinations thereof.

Embodiment 246. A pharmaceutical composition comprising the circular RNA polynucleotide of embodiment 243 and a transfer vehicle.

Embodiment 247. A method of producing circularized RNA, comprising transcribing a DNA polynucleotide sequence that is complementary to a precursor RNA polynucleotide of any one of embodiments 239-240.

Embodiment 248. A method of treating a subject in need thereof, comprising administering to the subject the pharmaceutical composition of embodiment 246 or the lipid nanoparticle of embodiment 245.

Embodiment 249. A linear precursor RNA polynucleotide comprising at least one modified A, C, G, or U nucleotide or nucleoside, wherein the linear precursor comprises at least a portion of each of:
a. a 5′ combined accessory element, comprising:
   i. a 3′ intron segment,
   ii. a 3′ exon segment,
b. an intervening region comprising an internal ribosome entry site (IRES) and a noncoding or coding region,
c. a 3′ combined accessory element, comprising:
   i. a 5′ exon segment, and
   ii. a 5′ intron segment.

Embodiment 250. The linear precursor RNA polynucleotide of embodiment 249, wherein the linear precursor maintains or improves circularization as compared to a corresponding linear precursor RNA polynucleotide comprising no nucleotide or nucleoside modifications.

Embodiment 251. A circular RNA polynucleotide comprising at least one modified A, C, G, or U nucleotide or nucleoside, wherein the circular RNA polynucleotide comprises:
a. a post-splicing 3′ exon segment,
b. optionally a 5′ internal homology region,
c. optionally a 5′ spacer,
d. an intervening region comprising an internal ribosome entry site (IRES) and a noncoding or coding region,
e. optionally a 3′ spacer,
f. optionally a 3′ internal homology region, and
g. a post-splicing 5′ exon segment.

Embodiment 252. The circular RNA of embodiment 251, wherein
a. the circular RNA reduces immunogenicity as compared to a corresponding circular RNA comprising no nucleotide or nucleoside modifications; and/or
b. the circular RNA maintains or improves translation of the coding region as compared to a corresponding circular RNA comprising no nucleotide or nucleoside modifications.

Embodiment 253. The linear precursor RNA polynucleotide or circular RNA of any one of embodiments 249-252, wherein between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides are modified.

Embodiment 254. The linear precursor RNA polynucleotide or circular RNA of any one of embodiments 249-252, wherein between 1% and 10% of the nucleotides or nucleosides are modified.

Embodiment 255. The linear precursor RNA polynucleotide or circular RNA of any one of embodiments 249-254, wherein:
a. the intervening region comprises no nucleotide or nucleoside modifications or is less than 10% modified;
b. the IRES comprises no nucleotide or nucleoside modifications or is less than 10% modified;
c. the noncoding or coding region comprises no nucleotide or nucleoside modifications or is less than 10% modified;
d. the 5′ intron segment and/or 3′ intron segment comprises no nucleotide or nucleoside modifications or is less than 10% modified;
e. the 5′ exon segment and/or 3′ exon segment comprises no nucleotide or nucleoside modifications or is less than 10% modified;
f. the 5′ spacer and/or 3′ spacer comprises no nucleotide or nucleoside modifications or is less than 10% modified; and/or
g. the 5′ homology region, 3′ homology region comprises no nucleotide or nucleoside modifications or is less than 10% modified.

Embodiment 256. The linear precursor RNA polynucleotide or circular RNA of any one of embodiments 249-255, wherein the modified nucleotide or nucleoside is selected from one or more of: m⁵U (5-methyluridine); m⁶A (N⁶-methyladenosine); s²U (2-thiouridine); Ψ (pseudouridine); Um (2′-O-methyluridine); m¹A (1-methyladenosine); m²A (2-methyladenosine); Am (2′-O-methyladenosine); ms² m⁶A (2-methylthio-N⁶-methyladenosine); i⁶A (N⁶-isopentenyladenosine); ms²i⁶A (2-methylthio-N⁶ isopentenyladenosine); io⁶A (N⁶-(cis-hydroxyisopentenyl)adenosine); ms²io⁶A (2-methylthio-N⁶-(cis-hydroxyisopentenyl)adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms² hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m² ₂G (N²,N²-dimethylguanosine); m²Gm (N²,2′-O-dimethylguanosine); m² ₂Gm (N²,N²,2′-O-trimethylguanosine); Gr(p) (2′-O-ribosylguanosine(phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mCmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵S2U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶ 2A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶ ₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm ⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine); pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-i-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-i-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, N1-methylpseudouridine, and N2,N2-dimethyl-6-thio-guanosine.

Embodiment 257. The linear precursor RNA polynucleotide or circular RNA of any one of embodiments 249-256, wherein the modified nucleotide or nucleoside is selected from one or more of: 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine, 5-(2-amino)propyl uridine, 5-halocytidine, 5-halouridine, 4-acetylcytidine, 1-methyladenosine, 2-methyladenosine, 3-methyicytidine, 6-methyluridine, 2-methylguanosine, 7-methylguanosine, 2,2-dimethylguanosine, 5-methylaminoethyluridine, 5-methyloxyuridine, 7-deaza-adenosine, 6-azouridine, 6-azocytidine, 6-azothymidine, 5-methyl-2-thiouridine, 2-thiouridine, 4-thiouridine, 2-thiocytidine, dihydrouridine, pseudouridine, queuosine, archaeosine, naphthyl substituted naphthyl groups, an O- and N-alkylated purines and pyrimidines, N6-methyladenosine, 5-methylcarbonylmethyluridine, uridine 5-oxyacetic acid, pyridine-4-one, pyridine-2-one, aminophenol, 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, N1-methylpseudouridine, and alkylcarbonylalkylated nucleotides.

Embodiment 258. The linear precursor RNA polynucleotide or circular RNA of any one of embodiments 249-257, wherein the modified nucleotide or nucleoside is selected from one or more of: 5-methylcytidine, 5-methoxyuridine, 1-methyl-pseudouridine, N6-methyladenosine, and/or pseudouridine.

Embodiment 259. A method of preparing a circular RNA comprising providing modified nucleotides or nucleosides to precursor RNA comprising:
a. a 5′ combined accessory element, comprising:
   i. a 3′ intron segment,
   ii. a 3′ exon segment,
b. an intervening region comprising an internal ribosome entry site (IRES) and a noncoding or coding region,
c. a 3′ combined accessory element, comprising:
   i. a 5′ exon segment, and
   ii. a 5′ intron segment.

Embodiment 260. A method of preparing a circular RNA comprising providing a first and second linear precursor RNA polynucleotide, wherein the first and second linear precursor RNA polynucleotides are capable of forming a circular RNA, and wherein either the first precursor or the second precursor but not both precursors comprises at least one modified A, C, G, or U nucleotide or nucleoside.

Embodiment 261. The method of embodiment 260, wherein the first precursor comprises a 3′ intron fragment of a first intron (Intron 1), a 5′ intron fragment of a second intron (Intron 2), a translation initiation element, the 5′ fragment of a sequence of interest, and two exon fragments that correspond with the intron fragments; and the second precursor comprises a 3′ intron fragment of the second intron (Intron 2), a 5′ intron fragment of the first intron (Intron 1), the 3′ fragment of the sequence of interest, and exon fragments corresponding to those in the first precursor.

Embodiment 262. The method of embodiment 261, wherein the TIE of the first precursor RNA polynucleotide comprises an IRES and a noncoding or coding region, and wherein the first and second precursor RNA polynucleotides optionally comprise spacers and/or homology arms.

Embodiment 263. The method of any one of embodiments 260-262, wherein between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the first linear precursor are modified.

Embodiment 264. The method of any one of embodiments 260-263, wherein in the first linear precursor:
a. the intervening region comprises no nucleotide or nucleoside modifications or is less than 10% modified;
b. the IRES comprises no nucleotide or nucleoside modifications or is less than 10% modified;
c. the noncoding or coding region comprises no nucleotide or nucleoside modifications or is less than 10% modified;
d. the 5′ intron segment and/or 3′ intron segment comprises no nucleotide or nucleoside modifications or is less than 10% modified;
e. the 5′ exon segment and/or 3′ exon segment comprises no nucleotide or nucleoside modifications or is less than 10% modified;
f. the 5′ spacer and/or 3′ spacer comprises no nucleotide or nucleoside modifications or is less than 10% modified; and/or
g. the 5′ homology region, 3′ homology region comprises no nucleotide or nucleoside modifications or is less than 10% modified.

Embodiment 265. The method of any one of embodiments 260-262, wherein between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the second linear precursor are modified.

Embodiment 266. The method of any one of embodiments 260-265, wherein in the second linear precursor:
a. the noncoding or coding region comprises no nucleotide or nucleoside modifications or is less than 10% modified;
b. the 5′ intron segment and/or 3′ intron segment comprises no nucleotide or nucleoside modifications or is less than 10% modified;
c. the 5′ exon segment and/or 3′ exon segment comprises no nucleotide or nucleoside modifications or is less than 10% modified;
d. the 5′ spacer and/or 3′ spacer comprises no nucleotide or nucleoside modifications or is less than 10% modified; and/or
e. the 5′ homology region, 3′ homology region comprises no nucleotide or nucleoside modifications or is less than 10% modified.

Embodiment 267. A modified circular RNA prepared from the methods of any one of embodiments 259-266.

Embodiment 268. A pharmaceutical composition comprising the circular RNA of any one of embodiments 251-258 or 267.

Embodiment 269. A method of treating a medical condition in a subject comprising administering the pharmaceutical composition of embodiment 268.

Embodiment 270. A method of preventing a disease or disorder in a subject comprising administering the pharmaceutical composition of embodiment 268.

### EXAMPLES

Wesselhoeft et al., (2019) RNA Circularization Diminishes Immunogenicity and Can Extend Translation Duration In vivo. Molecular Cell. 74(3), 508-520; Wesselhoeft et al., (2018) Engineering circular RNA for Potent and Stable Translation in Eukaryotic Cells. Nature Communications. 9, 2629; and M. Puttaraju et al, (1992) Group I permuted intron-exon (PIE) sequences self-splice to produce circular exons. Nucleic Acids Research. 20(20): 5357-5364 are incorporated by reference in their entirety.

The disclosure is further described in detail by reference to the following examples but are not intended to be limited to the following examples. These examples encompass any and all variations of the illustrations with the intention of providing those of ordinary skill in the art with complete disclosure and description of how to make and use the subject disclosure and are not intended to limit the scope of what is regarded as the disclosure.

### Example 1: Engineering of Permuted Intron Exon Synthesis from Group I and Group II Introns

a. Intron Quantification and Estimated Splicing

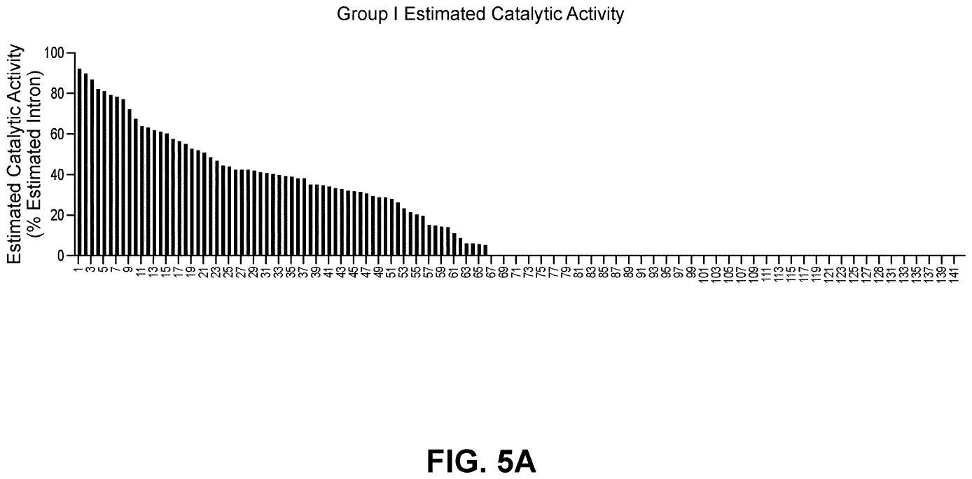

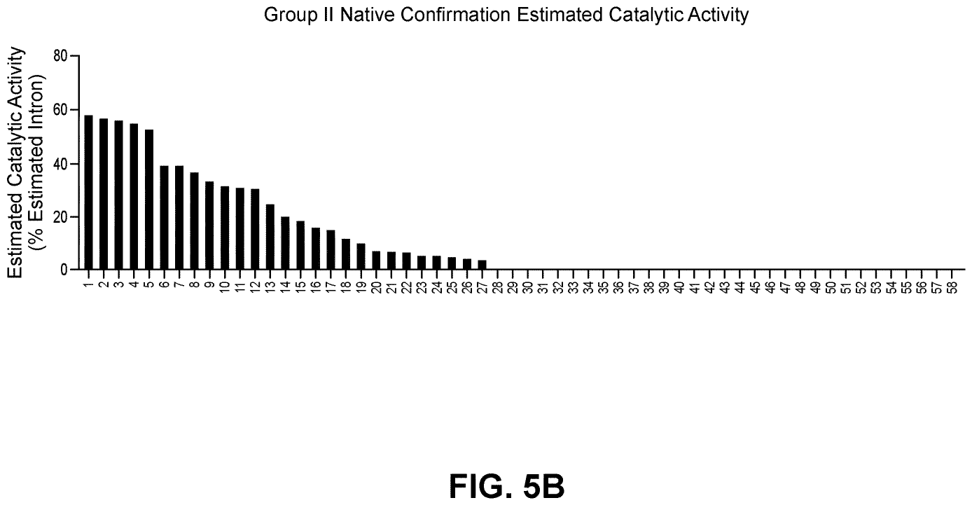

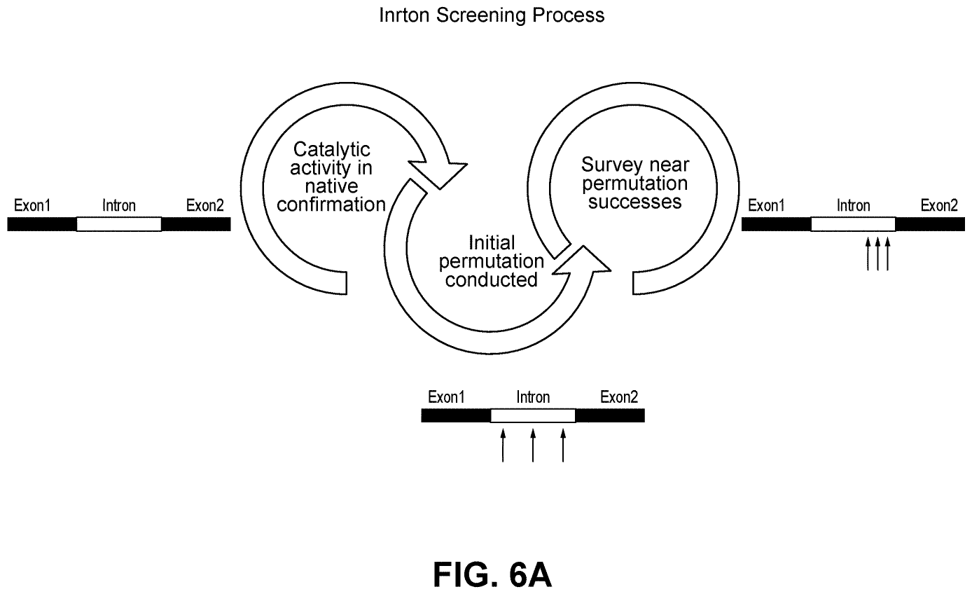

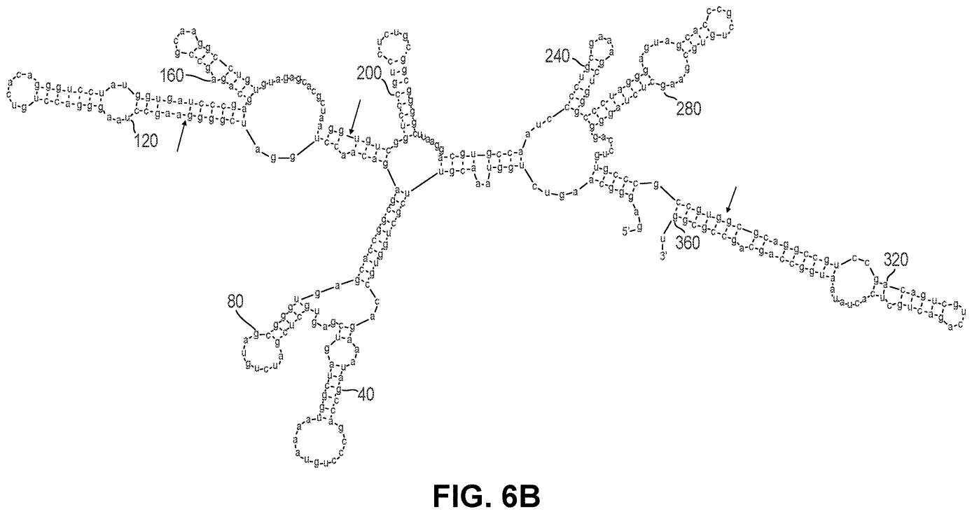

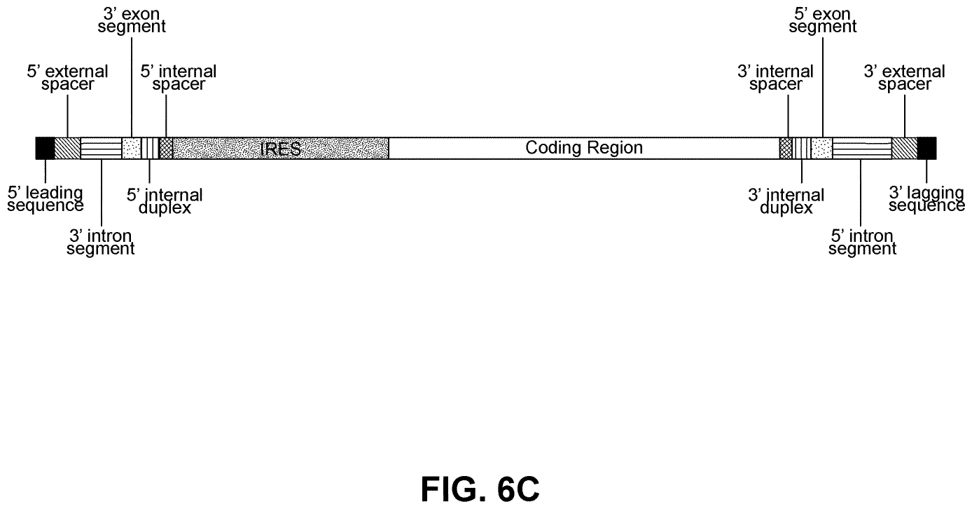

Plasmid DNA containing known group I or group II introns capable of splicing in a native context were identified from the Group I Intron Sequence and Structure Database (GISSD), GenBank, and other publicly available databases. The collected plasmid DNA sequences were further filtered to identify those comprising intron sequences less than 500 nucleotides and aligned to GenBank. An initial evaluation of the introns in their native confirmation (native intron) were tested for splicing activity as shown in FIG. 6a. In vitro transcription (IVT) reactions were performed on DNA templates with the native confirmation (e.g., comprising from the 5′ to 3′ order: a T7 polymerase promoter, the native 5′ exon, the native intron, the native 3′ exon, and a XbaI restriction site). The resulting solutions were purified via silica column purification. 15-20 μL of purified post-IVT solutions were analyzed at 16 minutes/sample using a commercially available SEC-HPLC (e.g., from Agilent Technologies, Lexington, MA) in 1× tris-EDTA running buffer solution at a pH of 6. The presence of splicing activity in each of the DNA templates was analyzed by viewing the number of peaks in the SEC-HPLC graphs (e.g., multiple peaks indicated multiple RNA species, which indicates some level of catalytic splicing activity). FIGS. 5a and 5b provide percent of introns present post IVT of the DNA templates. In FIGS. 5a and 5b, the DNA templates comprised a T7 promoter, the intron, corresponding exons, a Caprine kobuvirus internal ribosome entry site (IRES), a gaussia luciferase coding region, and a stop cassette.

From each plasmid DNA comprising group I introns or a fragment thereof, the group I introns along with at least 15 nucleotides from the 5′ and 3′ end of each intron (e.g., 15 nucleotides from the 5′ exon and 15 nucleotides from the 3′ exon) were extracted from the original plasmid DNA to form 5′ and 3′ intron and exon segments. Intron and exon segments comprised SEQ ID NOS: 2990-3130. From each plasmid DNA comprising group II introns or a fragment thereof, the group II introns along with at least 10 nucleotides from the 5′ and 3′ end of each intron (e.g., 10 nucleotides from the 5′ exon and 10 nucleotides from the 3′ exon) were extracted from the original plasmid DNA to form 5′ and 3′ intron and exon segments. Intron and exon segments comprised SEQ ID NOS: 3131-3187.

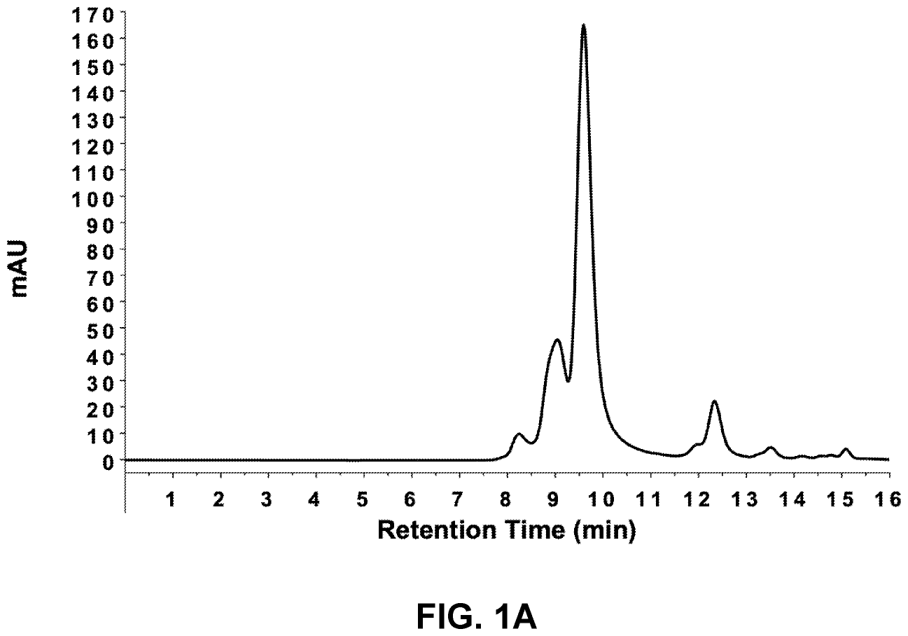

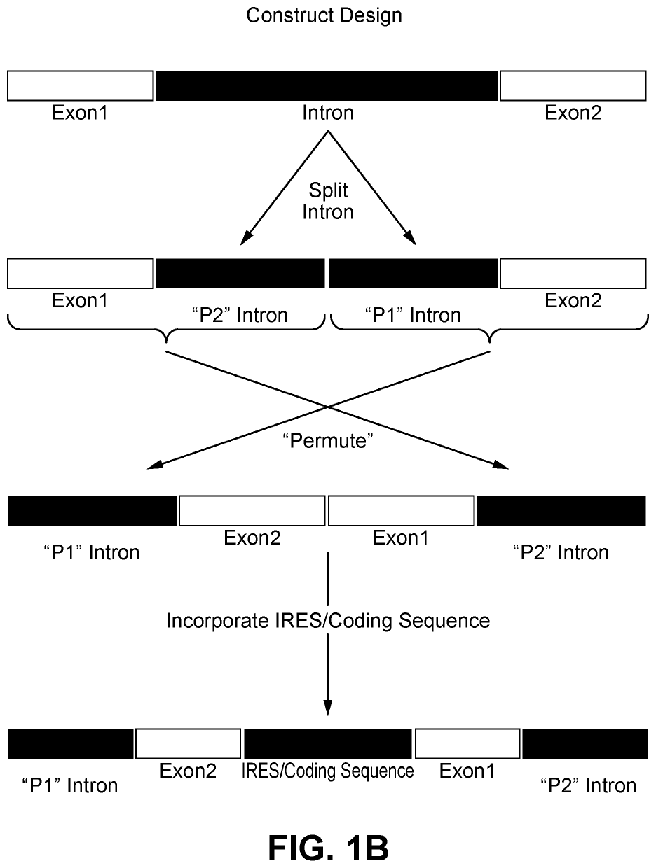

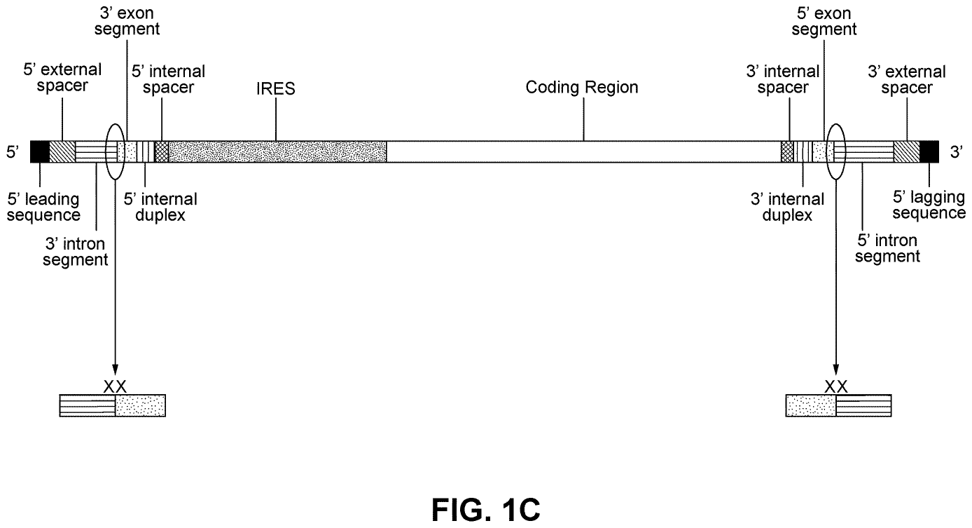

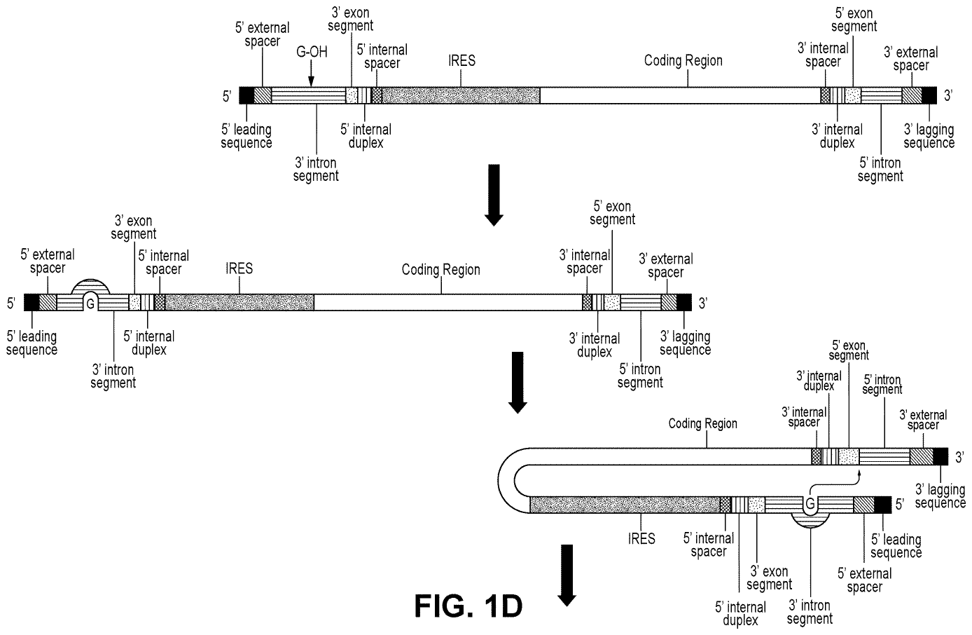

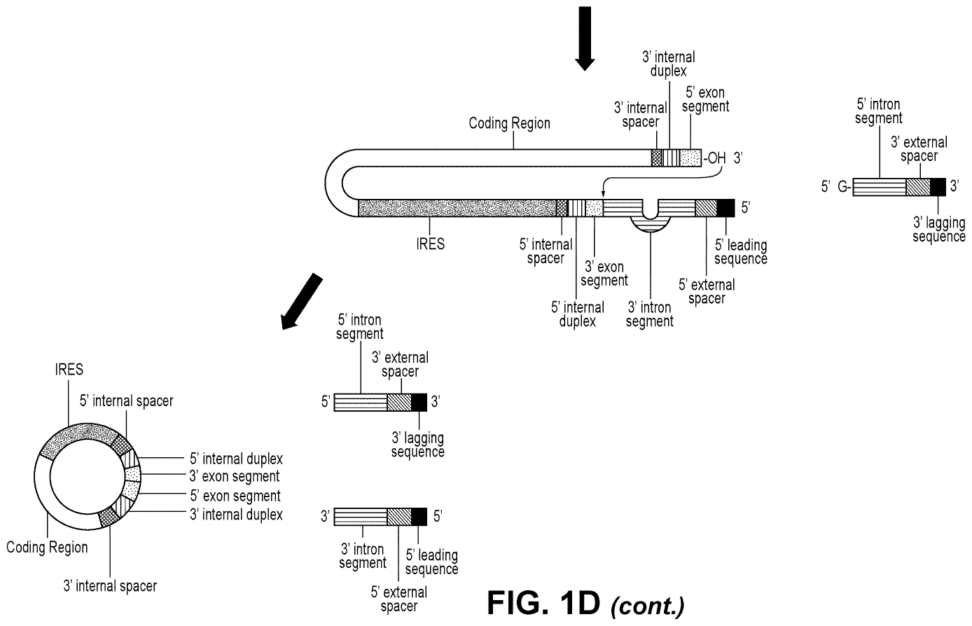

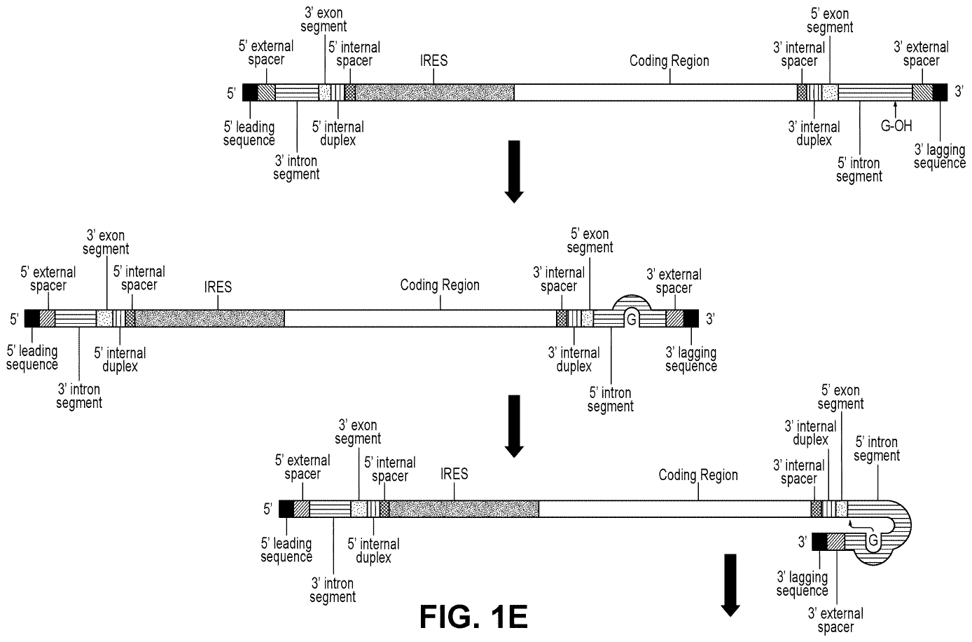

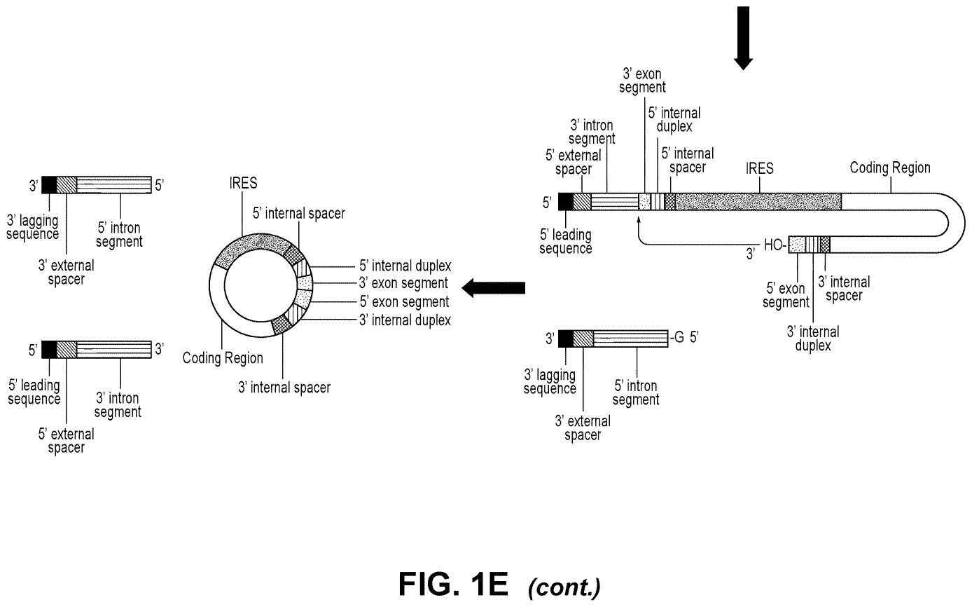

To determine splicing activity of the 5′ and 3′ intron and exon segments, DNA templates were created in the following 5′ to 3′ order using the 5′ and 3′ intron and exon segments: a T7 RNA polymerase promoter, a 3′ intron segment, 3′ exon segment, 5′ exon segment, 5′ intron segment, and a XbaI restriction site. In vitro transcription (IVT) reactions were performed on the DNA templates and resulting solutions were purified via silica column purification. 15-20 μL of purified post-IVT solutions were analyzed at 16 minutes/sample using a commercially available SEC-HPLC (e.g., from Agilent Technologies, Lexington, MA) in 1× tris-EDTA running buffer solution at a pH of 6. FIG. 1A illustrates SEC-HPLC analysis of an IVT reaction for DNA templates comprising 5′ and 3′ intron and exon segments from Anabaena (SEQ ID NO: 2990). Splicing activity was calculated based on either the peaks containing introns (IntronPeak method) or the peaks containing circular RNA (CircPeak method). From FIG. 1A, the two peaks on the right from the largest peak depicting the spliced-out 5′ and 3′ intron segments (beginning approximately at 11.5 and 13 minutes) were used to estimate percent spicing by calculating the % intron contribution (i.e., area under the spliced-out 5′ and 3′ intron fragments) to the total RNA area under the curve (i.e., IntronPeak Method) or by calculating the % circular RNA concentration.

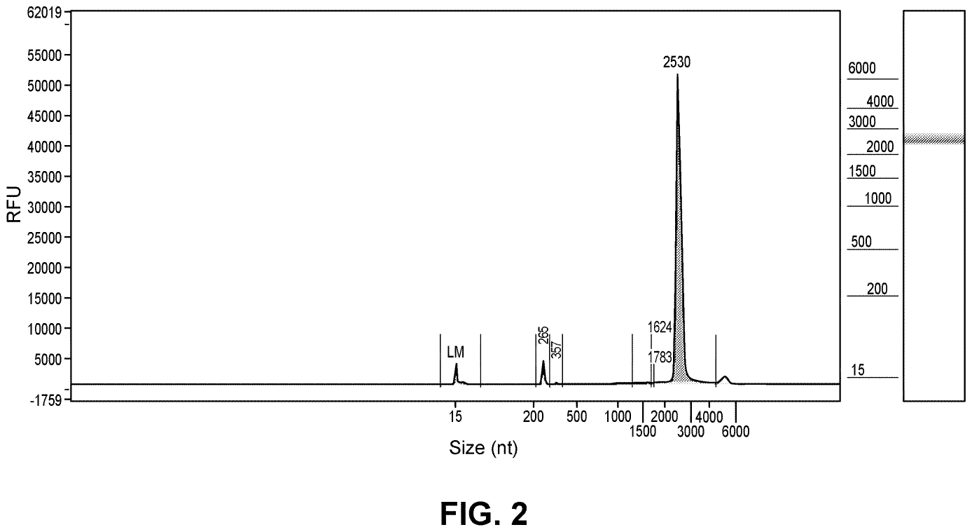

In tandem, analysis was conducted on the purified post IVT reactions solutions using a fragment analyzer. 5 μL sample of the purified post-IVT solutions and 12 μL of 100% formaldehyde were prepared. To each well containing the RNA template sample and formaldehyde, 12 μL of a diluent marker was added. The resulting solution was analyzed using a commercially available fragment analyzer (e.g., Agilent DNF-471 RNA kit). The fragment analyzer was allowed to run for 1 hour and then the results were collected. Results of the fragment analyzer of the post-IVT reaction solution created from DNA templates comprising Anabaena intron and exon segments are depicted in FIG. 2. In FIG. 2, the peaks at 265 and 357 nucleotides correspond to the spliced out 5′ and 3′ intron segments. The peak at 2530 corresponds to the circular RNAs along with other non-intron segments. Percent intron concentration using the IntronPeak method was calculated from the fragment analyzer. The corresponding concentrations of each of the peaks for FIG. 2, including the percent intron concentration, is depicted in Table 4 below. Percent estimated circularization (e.g., estimated percent splicing) was determined from the percent intron concentration and the percent of theoretical introns (i.e., total number of intron nucleotides/total number of nucleotides in the total number of nucleotides present in the purified post-IVT solution).

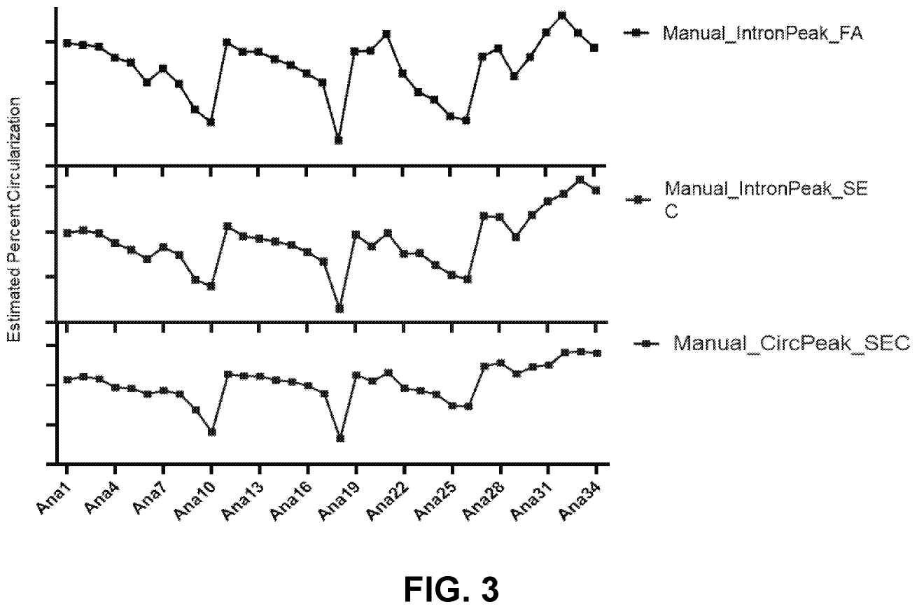

FIG. 3 provides the resulting estimated percent circularization from 34 DNA templates comprising different Anabaena intron fragments that underwent IVT (SEQ ID NOS: 3188-3221) and was then subsequently analyzed using either the fragment analyzer and/or SEC-HPLC. The Spearman Correlation of Rank and Pearson correlation between the estimated percent circularization generated by the fragment analyzer results was 0.907716 and the SEC-HPLC results were 0.89331 respectively.

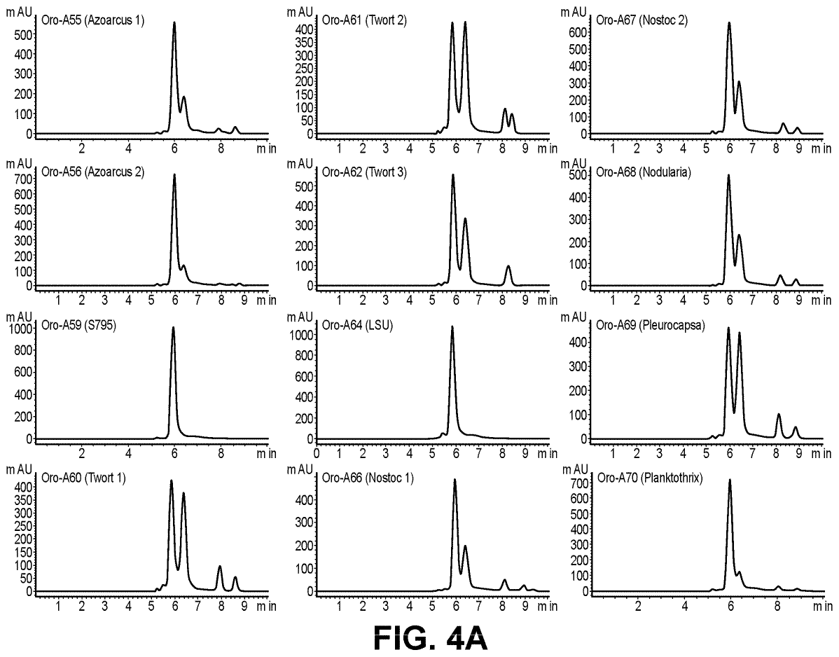

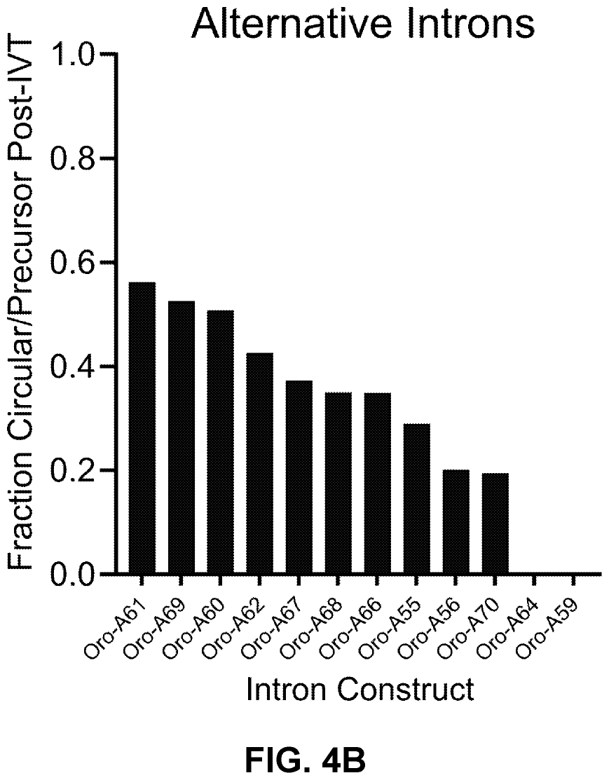

FIGS. 4a and 4b provides the ratio of circular products: precursor formed post IVT of DNA templates comprising various, non-Anabaena intron fragments. In FIG. 4b, the DNA templates used to generate the estimated percent circularization comprised a CBV3 internal ribosome entry site (IRES), gaussia luciferase coding region, spacers, internal and external homology regions. Percent introns present was measured by calculating number of excised introns per total of excised introns and precursor RNA remaining. The introns depicted in FIGS. 5a and 5b are from Group I and Group II intron classifications.

TABLE 4

| | | | | Relative |
|---|---|---|---|---|
| | | | | Concentration |
| From size (nt) | To size (nt) | RFU | Concentration | Percent (%) |
| 0 | 75 | 3196 | 0.3163 | — |
| 218 | 317 | 3631 | 3.1215 | 3.7 |
| 317 | 400 | 130 | 0.0983 | 0.1 |
| 1305 | 1696 | 259 | 0.08546 | 1.0 |
| 1696 | 1790 | 317 | 0.2645 | 0.3 |
| 1790 | 4594 | 50981 | 80.8742 | 94.9 |

b. Group I and II intron Permutations

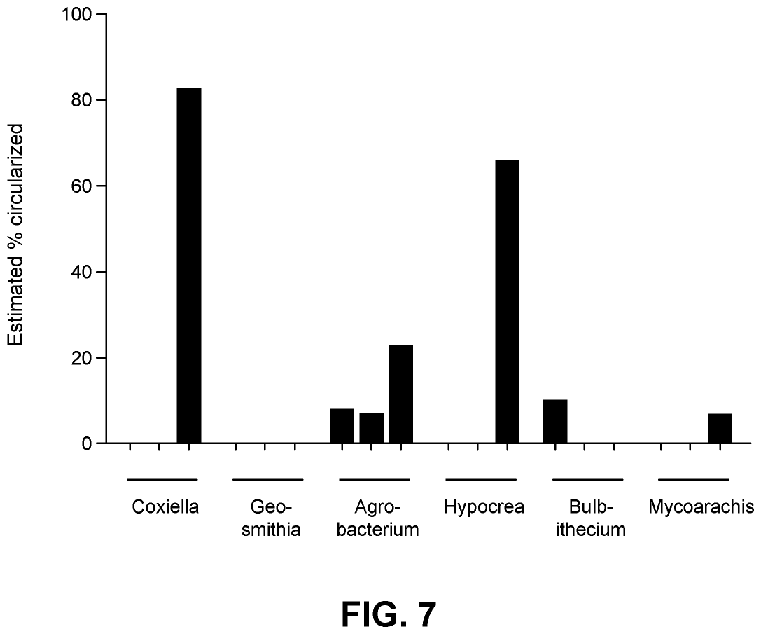

Plasmid DNA containing known group I or group II introns capable of splicing in a native context as described in Example 1a (above) were permuted in three different locations (e.g., the 5′ terminus, middle and 3′ terminus) as illustrated in FIGS. 6a and 6b. In some embodiments, other locations are permuted. Permutation locations were selected using RNAFold prediction to minimize impact of permutation to overall structure and functionality of the intron. The permuted intron sequences and exon segment sequences (e.g., exons segments extracted from the original plasmid DNA with at least 15 nucleotides from the 5′ and 3′ end) were collected to form 5′ and 3′ permuted intron and exon (PIE) segments. A T7 RNA polymerase was added before the PIE construct, and a XbaI restriction site was added after the PIE construct to form a DNA template as illustrated in FIG. 6c. IVT reactions were performed on the DNA templates. IVT reactions were performed on the DNA templates and resulting solutions were purified via silica column purification Purified IVTs products were analyzed by SEC-HPLC in 1× tris-EDTA running buffer (Agilent), pH6, to determine splicing activity. Results for 6 of the intron constructs tested are shown in FIG. 7.

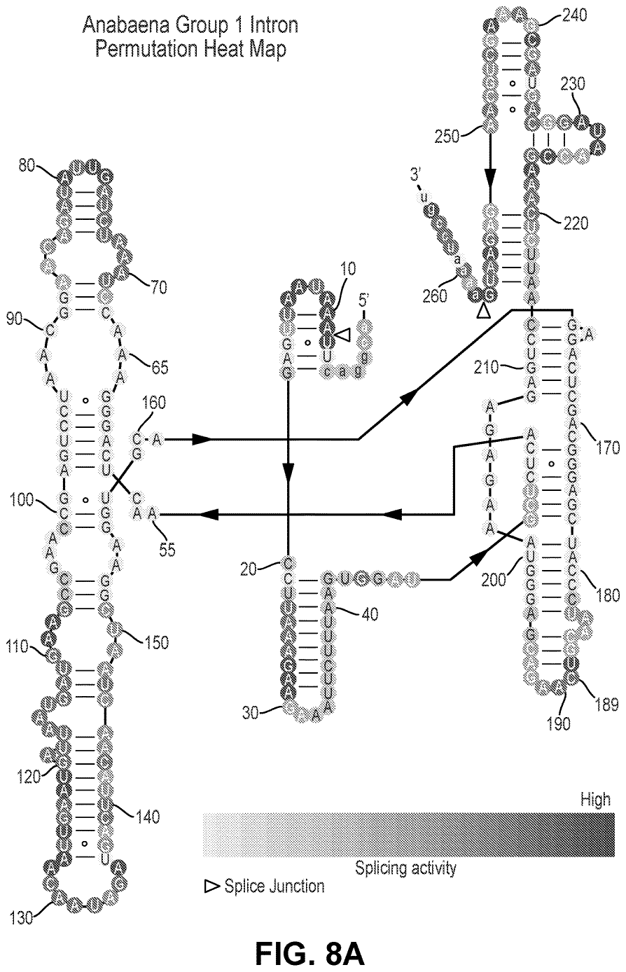

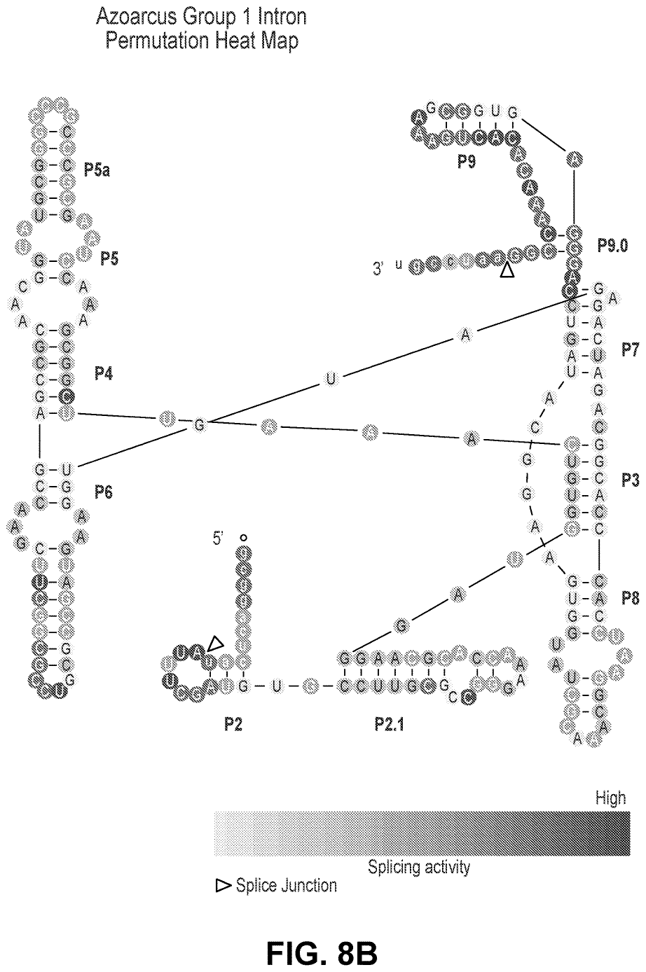

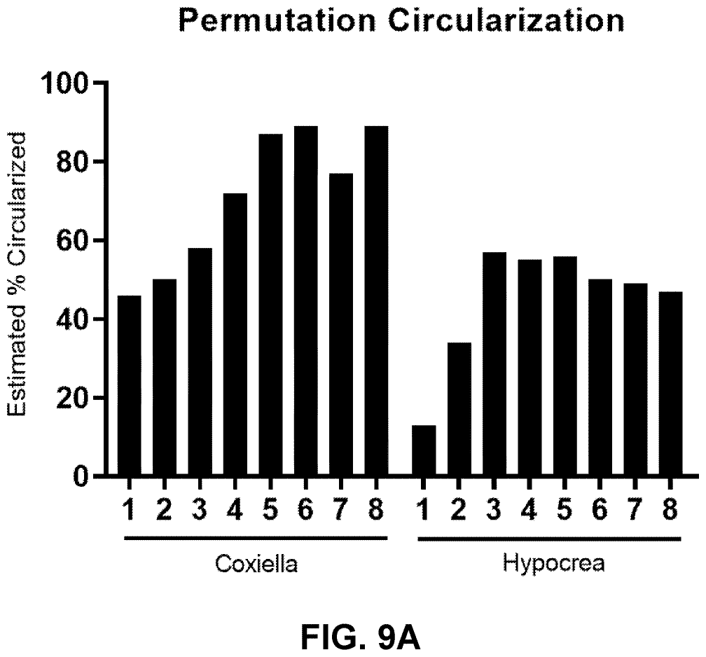

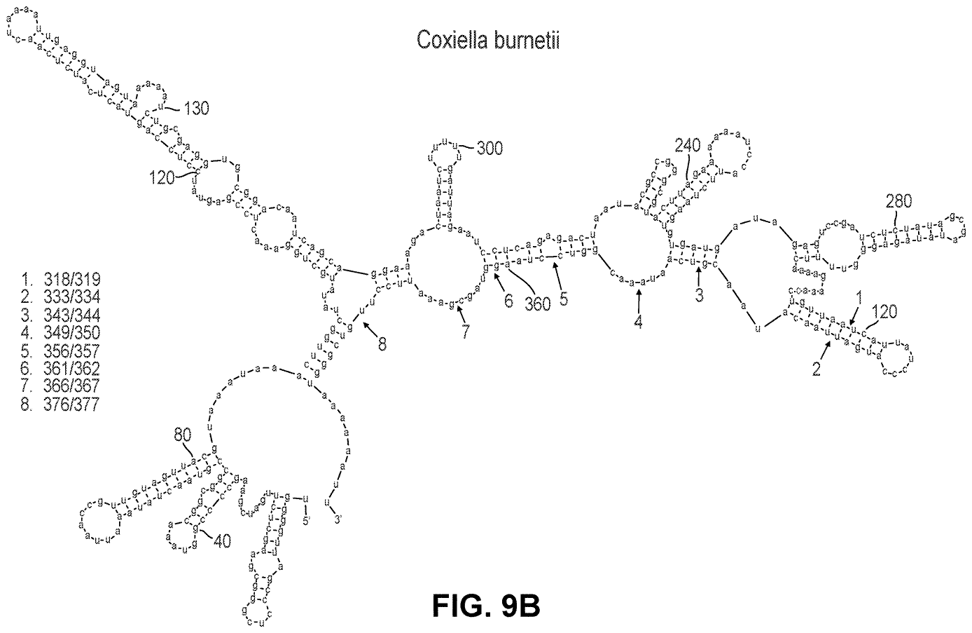

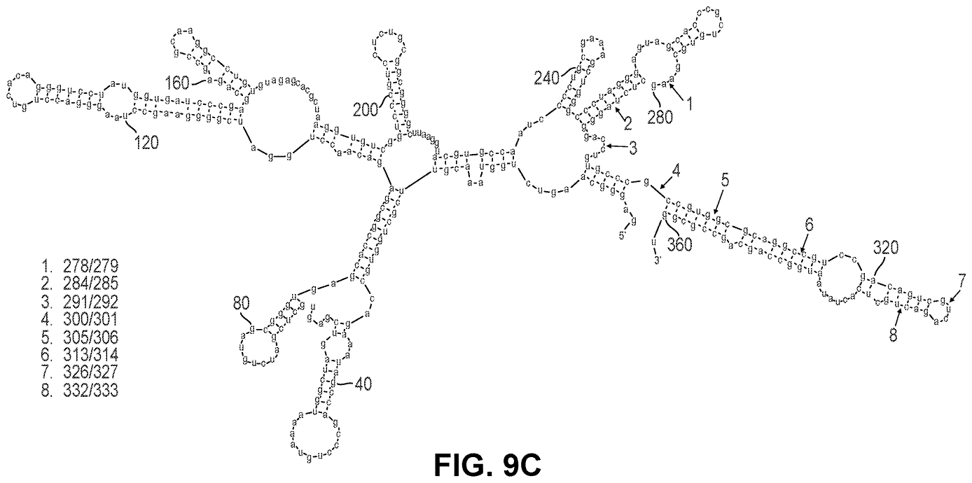

Permuted introns that showed circularization activity in the preliminary assessment of a small number of permutations (i.e., the three different locations described in the paragraph above) were permuted at more sites to determine optimal permutation site(s). For example, Anabaena Group I introns were permuted to find optimal splicing activity. For each of the permutation sites, IVT reactions were performed on DNA templates comprising (1) a T7 polymerase promoter, (2) 5′ and 3′ permuted intron segments formed from the permuted site(s), (3) 5′ and 3′ exon segments, and (4) an XbaI restriction site. IVT reactions were performed on the DNA templates and resulting solutions were purified via silica column purification. Purified IVTs products were analyzed by SEC-HPLC in 1× tris-EDTA running buffer (Agilent), pH6, to determine splicing activity. FIG. 8 provides a heat map with the splicing activity of the various permuted sites tested along the Anabaena Group I intron. Each location in FIG. 8a corresponds with a permuted intron sequence (SEQ ID NOS: 3222-3483) that was used to create permuted intron exon sequences for the DNA templates. Splicing activity by intronic structural feature for the Anabaena intron is shown in Table 5 below. FIG. 9A provides the percent circularization (e.g., estimated percent splicing) for Coxiella and Hypocrea introns at 8 different permutation locations.

TABLE 5
Permutation Location

| | | | 3′ Base | | | |
|---|---|---|---|---|---|---|
| | | | A | C | G | U |
| Internal Loop | 5′ Base | A | 15.78% | 0.00% | 0.00% | — |
| | | C | 0.00% | 0.93% | | 0.00% |
| | | G | 13.77% | 0.00% | | |
| | | U | | | 0.00% | |
| | | | 3′ Base | | | |
| | | | A | C | G | U |
| Middle Loop | 5′ Base | A | 19.85% | 6.50% | 21.93% | 24.83% |
| | | C | 13.69% | | | |
| | | G | | 0.70% | | 38.05% |
| | | U | 38.98% | | | 37.59% |
| | | | 3′ Base | | | |
| | | | A | C | G | U |
| Stem | 5′ Base | A | 30.23% | 16.94% | 16.84% | 17.79% |
| | | C | 17.22% | 9.90% | 20.30% | 12.56% |
| | | G | 11.90% | 14.23% | 7.89% | 18.42% |
| | | U | 27.97% | 13.17% | 15.93% | 13.71% |
| | | | 3′ Base | | | |
| | | | A | C | G | U |
| Terminal Loop | 5′ Base | A | 24.94% | 21.35% | 34.22% | 36.33% |
| | | C | 35.27% | | 0.00% | |
| | | G | 19.21% | | 0.00% | 48.26% |
| | | U | 40.14% | | | 45.47% |

### Example 2: Monotron and Terminal Sequence Design

a. Exon Minimizations

DNA templates constructs were developed with various exon lengths and analyzed for estimated percent splicing. In some constructs, the exons were derived from DNA templates with group I introns. Each of the DNA templates comprised intron segments, exon segments, an internal ribosome entry sites (TRES) and a firefly luciferase coding region. Estimated percent splicing was calculated according to the methods described in Example 1. Intron segments were derived from Anabaena or Coxiella burnetti group I introns; exon segments were derived from the same species as the introns. Full-length or non-minimized exon segments were used as a positive control (e.g., 5′ full-length Anabaena exon segments comprised 51 nucleotides and 3′ full-length Anabaena exon segments comprised 15 nucleotides). Minimized exon segments were constructed from progressively deleting one or more nucleotides from either the 5′ or 3′ terminal of the full-length exon segments until the exon consisted of a single nucleotide of the splice site dinucleotide. The DNA templates for the exon minimizations comprised a sequence from SEQ ID NOS: 3189-3205, 3579-3596, and 3642-3664.

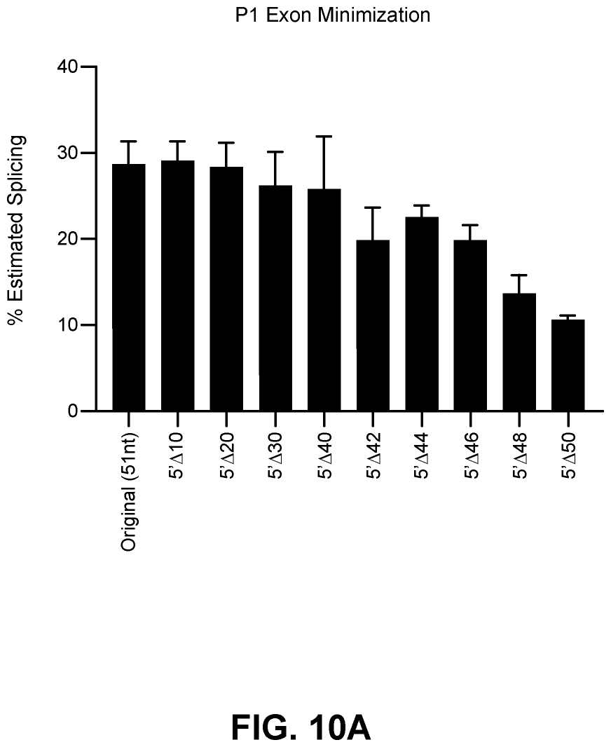

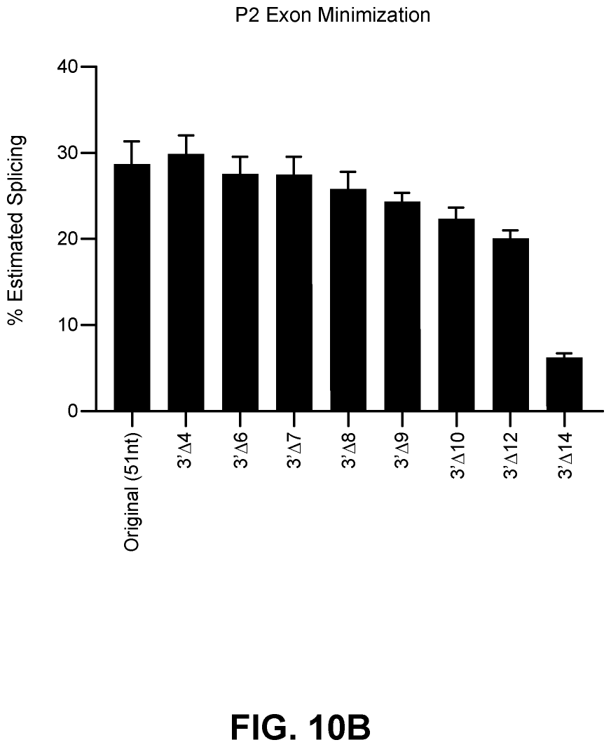

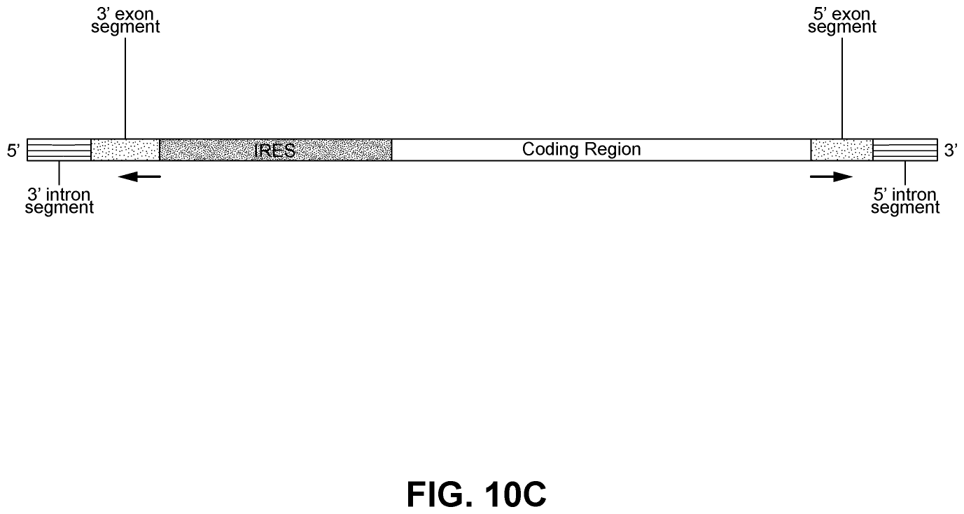

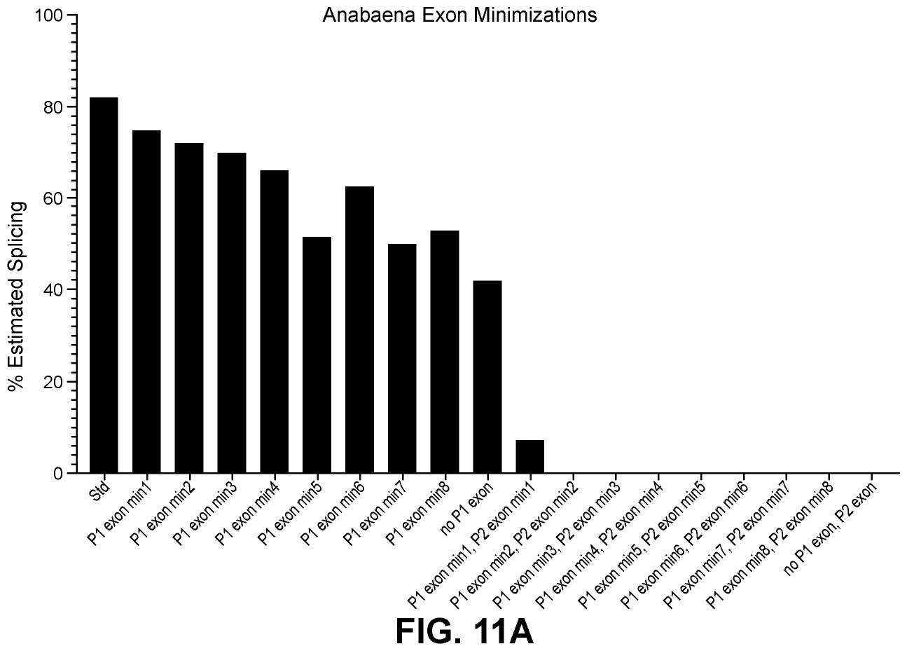

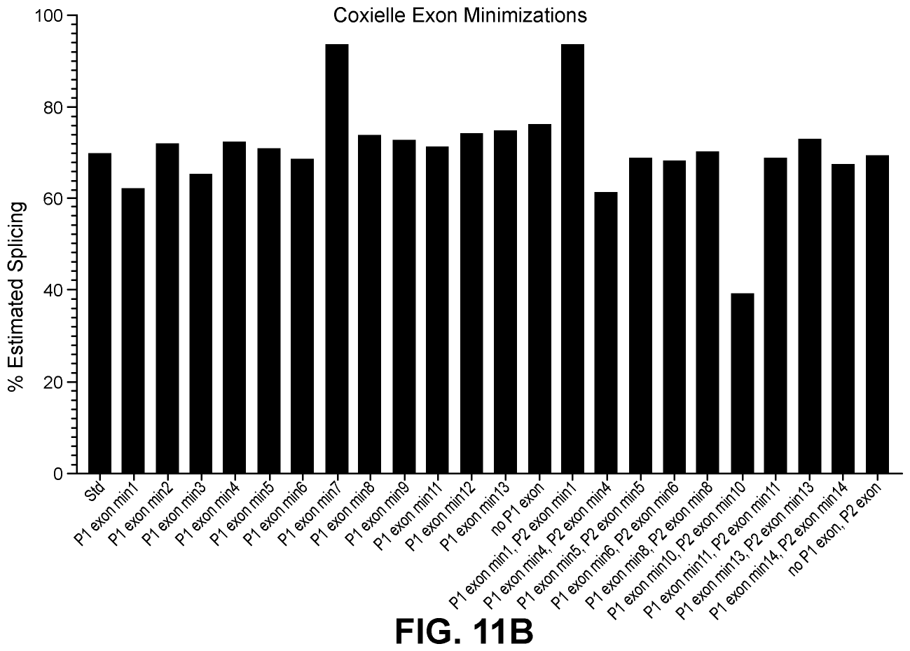

Results from the nucleotide deletions on Anabaena exon segments can be seen in FIGS. 10a-b and 11a; corresponding nucleotide deletions for the Coxiella burnetti exon segments can be seen in FIG. 11b. In FIGS. 10a-b, Anabaena introns and exon segments were used to create the permuted intron exon segments in the plasmid DNAs used for analyzing estimated percent splicing. Minimized exon segments in FIG. 10a were designed from deleting one or more nucleotides from the 5′ terminal of a 5′ exon segment (e.g., deletions ranged from 10 through 50 nucleotides). In FIG. 10b exon segments were designed from deleting one or more nucleotides from the 3′ terminal of a 3′ exon segment (e.g., deletions ranged from 4 to 14 nucleotides). The constructs described in FIG. 11a contained minimized exon segments developed by deleting one or more nucleotides from the permuted Anabaena exon with initial permutation at position 230 in FIG. 8. The constructs described in FIG. 11b contained minimized exon segments developed by deleting one or more nucleotides from Coxiella burnetti exons with initial intron permutation at a naturally occurring splice junction. The constructs described in FIGS. 11a and b contain unminimized 3′ exons (depicted as P1) and 5′ exons (depicted as P2) comprising 9 and 8 nucleotides respectively. Nucleotide deletions were conducted on either the 3′ and/or 5′ exons. 3′ exon deletions were conducted incrementally from the 3′ terminus of said exon sequence; 5′ exon deletions were conducted incrementally from the 5′ terminus of said exon sequence.

Plasmid DNA constructs will be developed with various exon lengths and analyzed for estimated percent splicing and evaluated in accordance with the procedure above, where the exons are derived from DNA plasmids with group II introns.

b. Sequence Scrambles

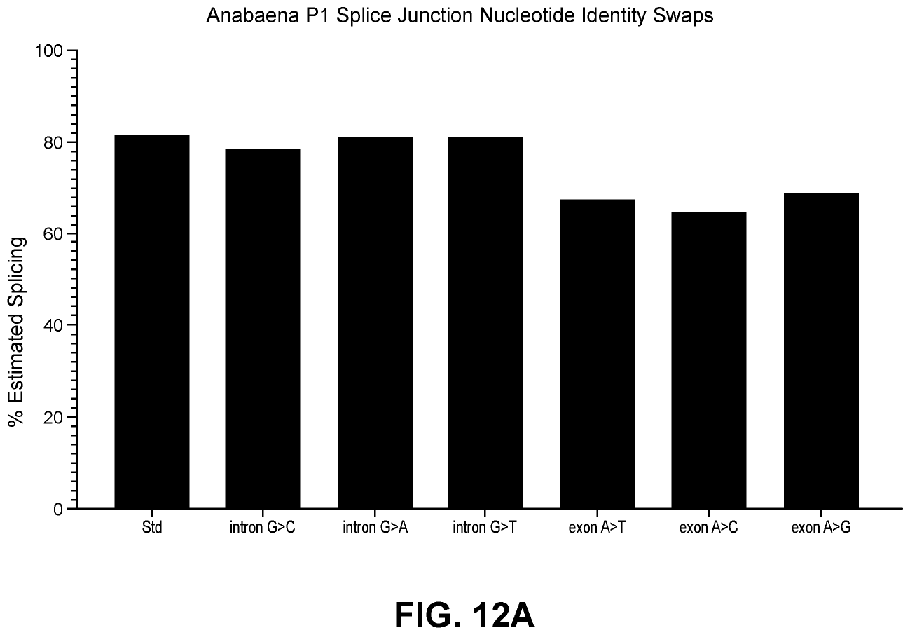

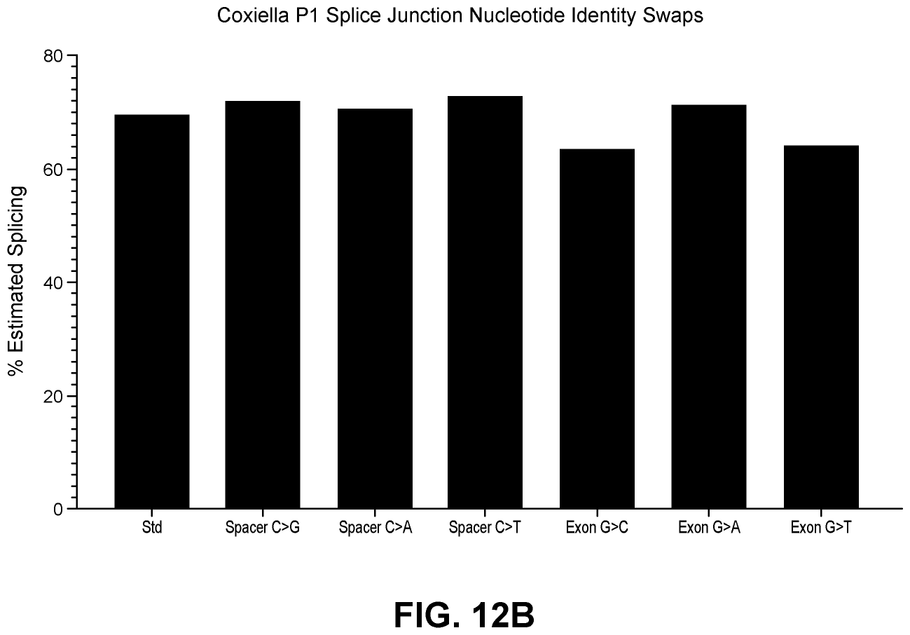

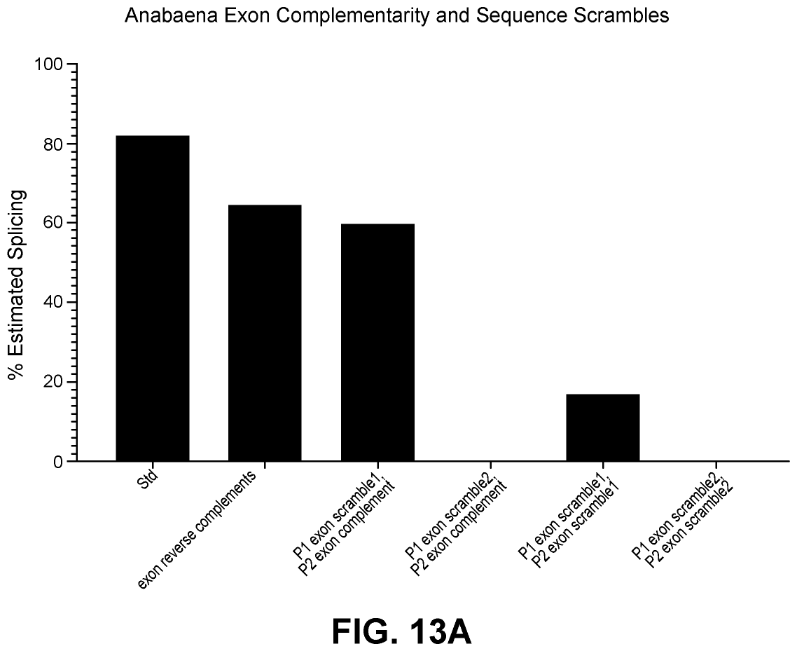

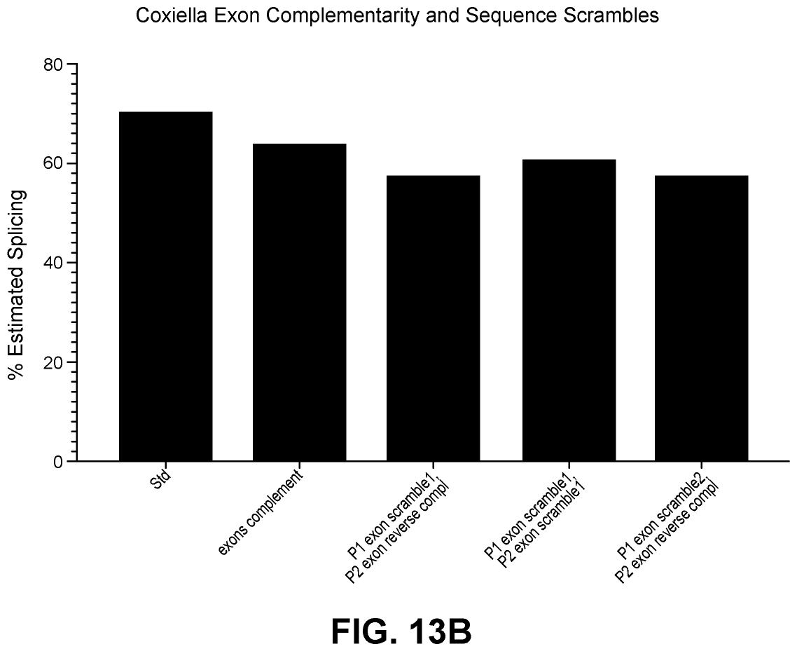

Circularization function was analyzed for various RNA constructs comprising one or more nucleotide base changes from the native permuted intron exon elements. Precursor DNA templates of the constructs were developed from sequences with either native Group I or Group II introns with self-splicing capabilities. The DNA templates were designed to be in the following order a T7 promoter sequence, a 5′ external homology arm sequence, a 5′ external spacer sequence, 3′ group I or Group II intron and exon sequences, a 5′ internal homology arm sequence, a 5′ internal spacer sequence, a CVB3 internal ribosome entry site (IRES), an expression sequence encoding firefly luciferase (fluc), a stop codon, a 3 internal spacer sequence, a 3′ internal homology arm sequence, 5′ Group I or Group II intron and exon sequences. The introns and exons of these DNA templates were prepared with one or more nucleotide base changes including or more of the following changes: inclusion of flanking splice site junction base swaps in the intron and/exon sequences, exon scrambles, partially or non-complementary paired element, or base pair reverse complement swaps. Base identity of the splice junctions was investigated by changing each base to one of the other three bases within the context of the known base. DNA templates were comprised of a sequence from SEQ ID NOS: 3622, 3624-3627, 3635-3641 and 3665-3668. In vitro transcription reactions of the DNA templates and subsequent percent estimated splicing were performed according to Example 1. Estimated percent splicing results for the constructs are depicted in FIGS. 12a, 12b, 13a, and 13b.

c. Intron Deletions

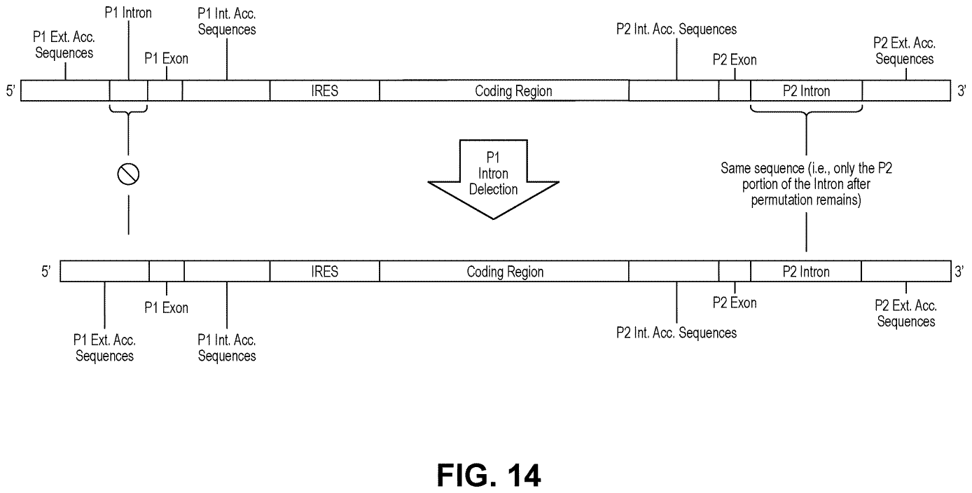

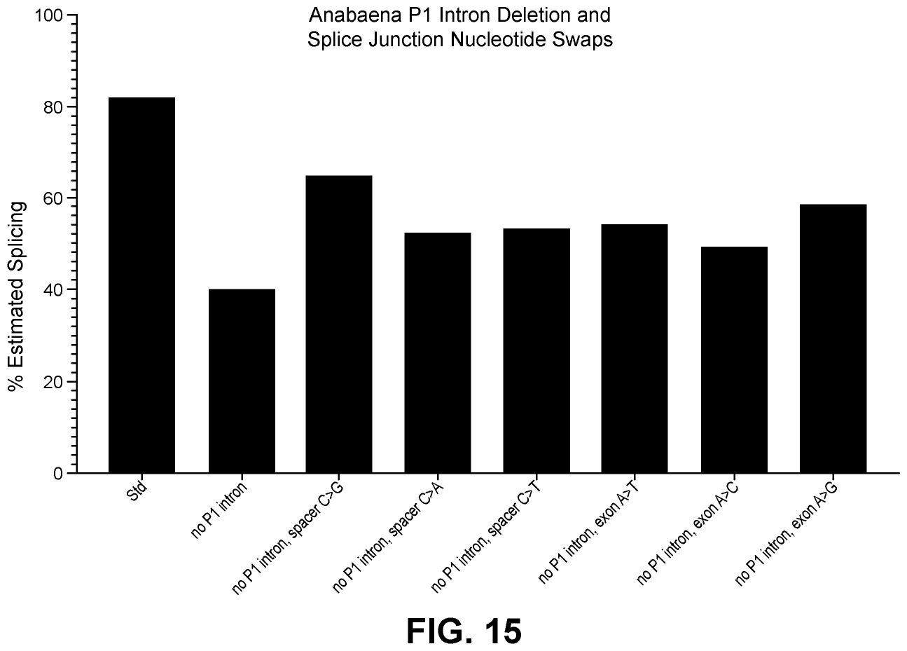

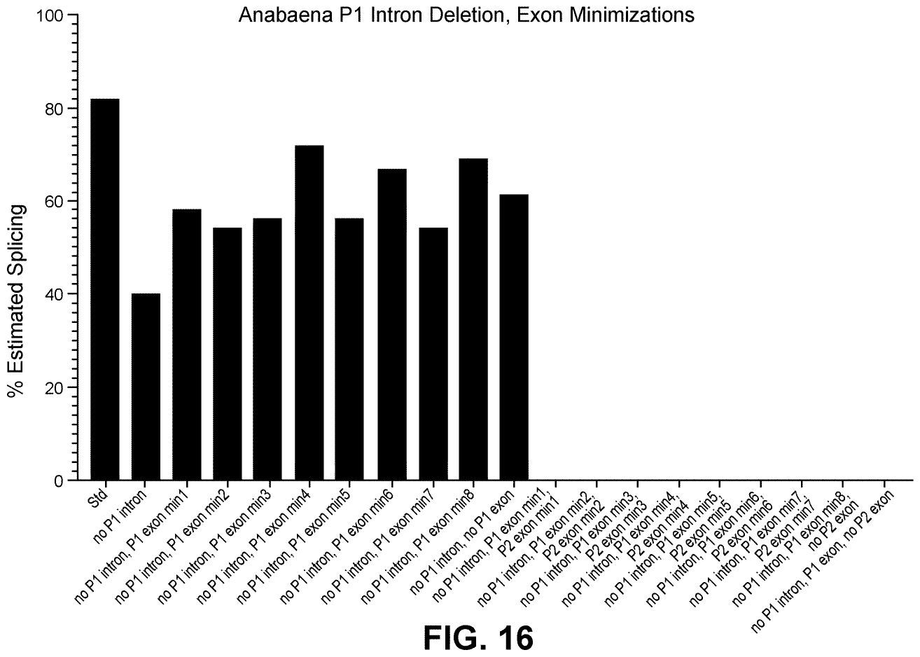

DNA templates comprising two permuted intron-exon segments were compared with DNA templates with a permuted intron-exon segment and an additional exon segment (i.e., lacking an intron segment adjacent to the exon segment), e.g., SEQ ID NOS: 3597-3603 (see FIG. 15), and SEQ ID NOS: 3597 and 3604-3621 (see FIG. 16). An example of the intron deletion in the DNA template can be seen in FIG. 14. As depicted in FIG. 14, permuted 3′ intron segments were deleted near the 5′ end of the construct. In some of the constructs, one of more of the DNA constructs received splice site nucleotide base changes. In other of the constructs, 3′ exons and/or 5′ exons were minimized incrementally one nucleotide at a time from the 3′ terminus or 5′ terminus of the 3′ exons or 5′ exon respectively. Base identity of the splice junction was investigated by changing each base to one of the other three bases within the context of the known. Exemplary DNA templates were derived from Anabaena naturally occurring plasmids and contained CBV3 IRESes along with expression sequences encoding firefly luciferase. Additional spacers and homology arms were added to the templates to enhance circularization. Estimated percent circularization for each of the DNA templates were analyzed based on the methods provided in Example 1. Results from the DNA templates are depicted in FIGS. 15 and 16.

d. Constructs Comprising Monotron and Terminal Sequences

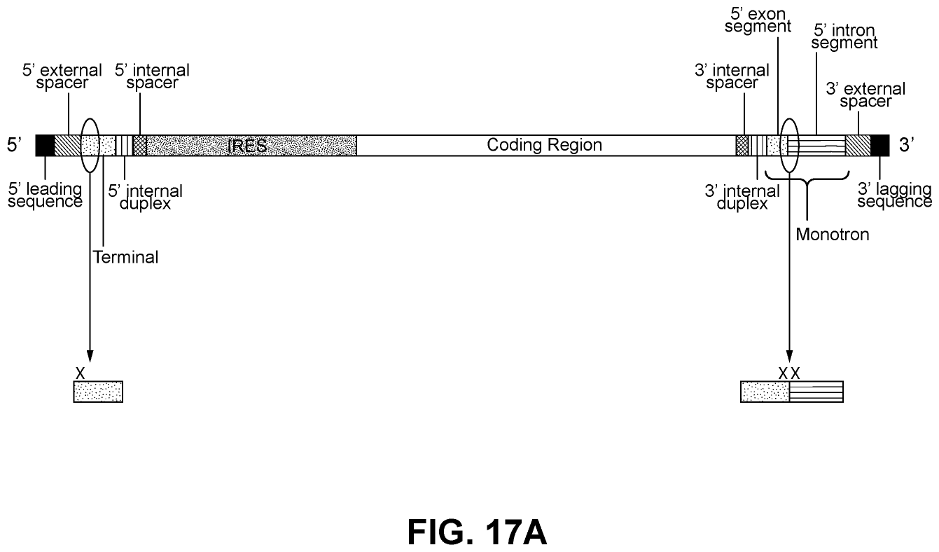

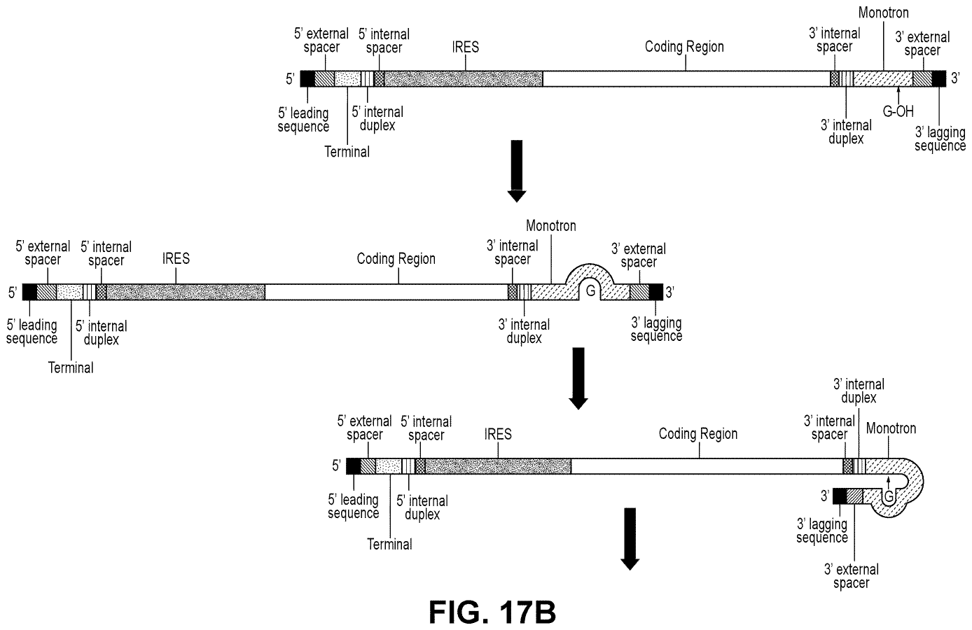

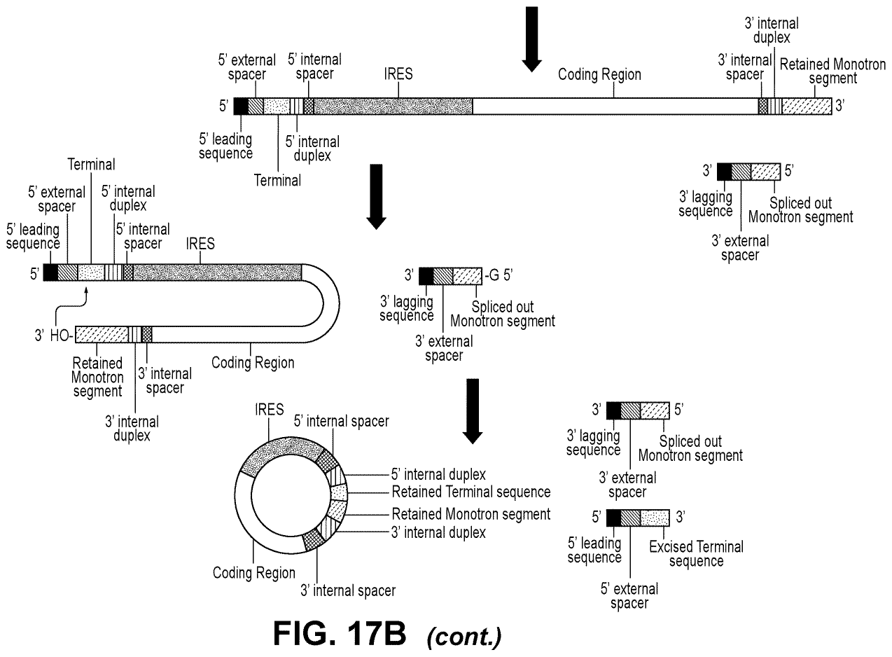

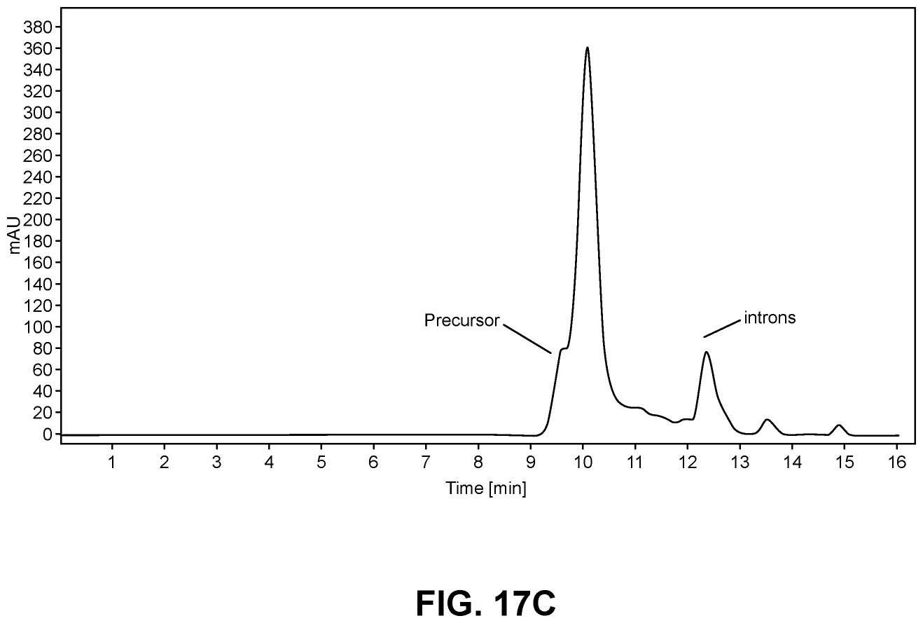

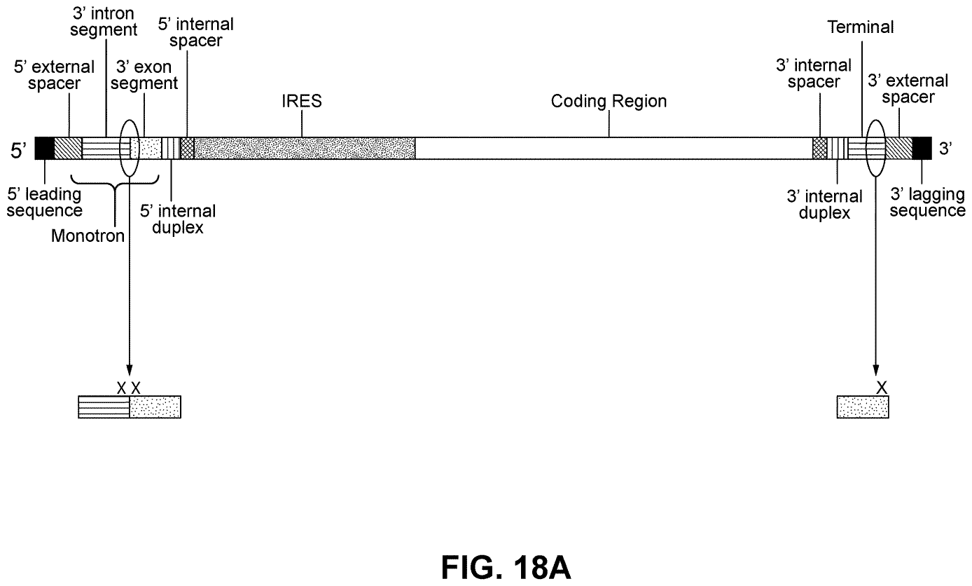

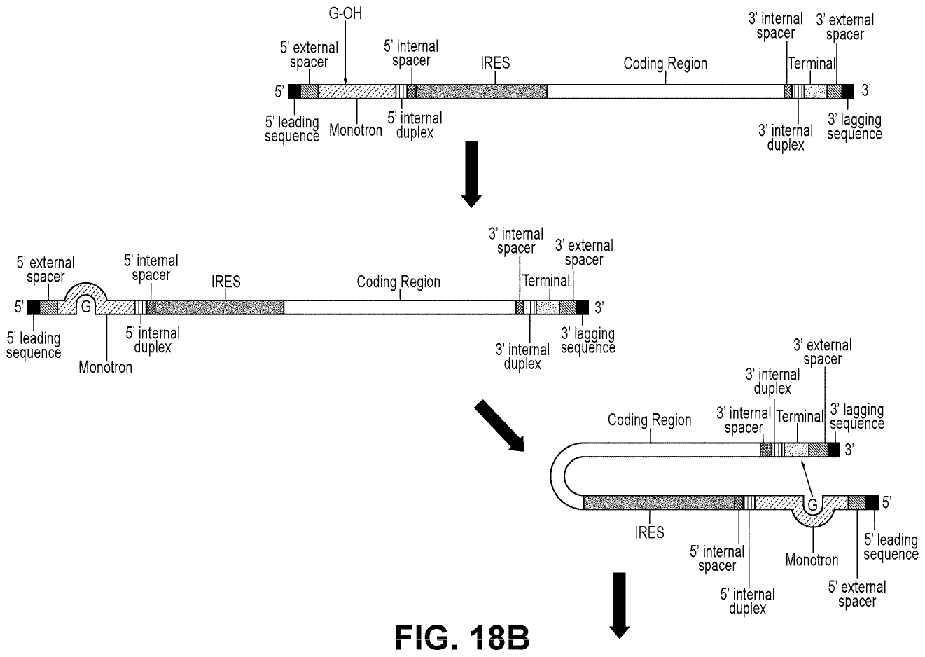

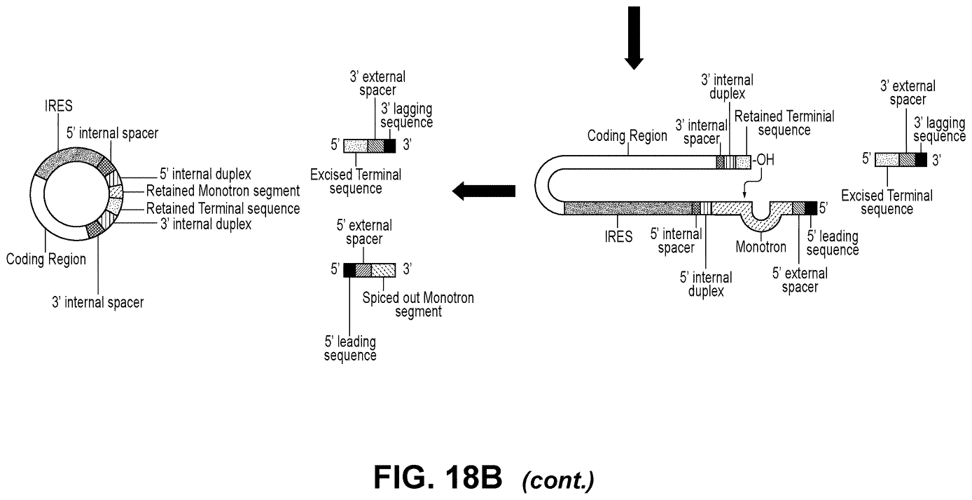

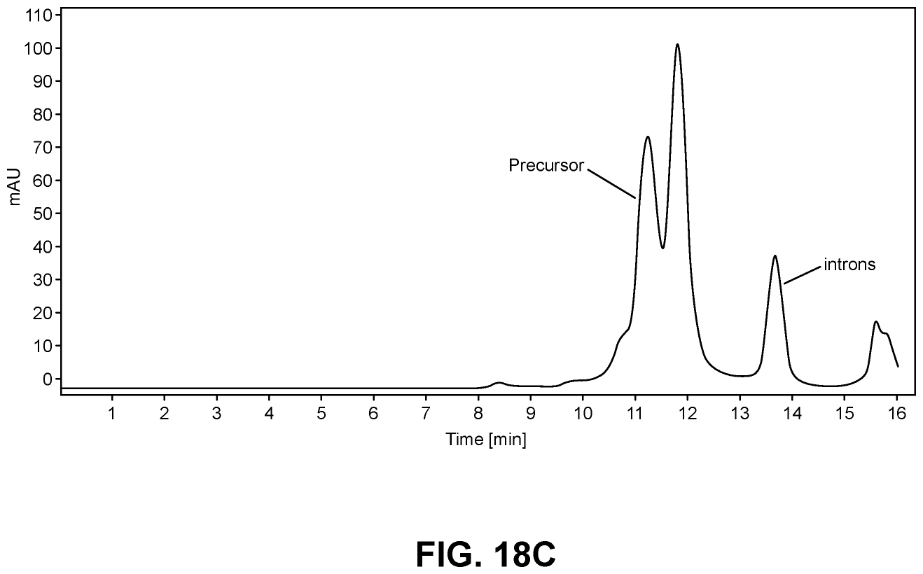

DNA templates were designed to have either a monotron sequence 5′ to the internal ribosome entry site (IRES) and the terminal sequence downstream to the montron sequence or vice versa (depicted in FIGS. 17a and 18a). The DNA templates underwent in vitro transcription (IVT) reactions as described in Example 1. The linear precursor RNAs formed from the DNA templates were allowed to undergo splicing to create circular RNA products and spliced out biproducts as illustrated in FIGS. 17b and 18b. The DNA templates comprised a sequence from SEQ ID NOS: 3229 or 3476. The post-IVT reaction solutions of DNA templates were analyzed using a size exclusion-high-performance liquid chromatography (SEC-HPLC) for both constructs. Results from the SEC-HPLCs for the constructs are provided in FIGS. 17c and 18c.

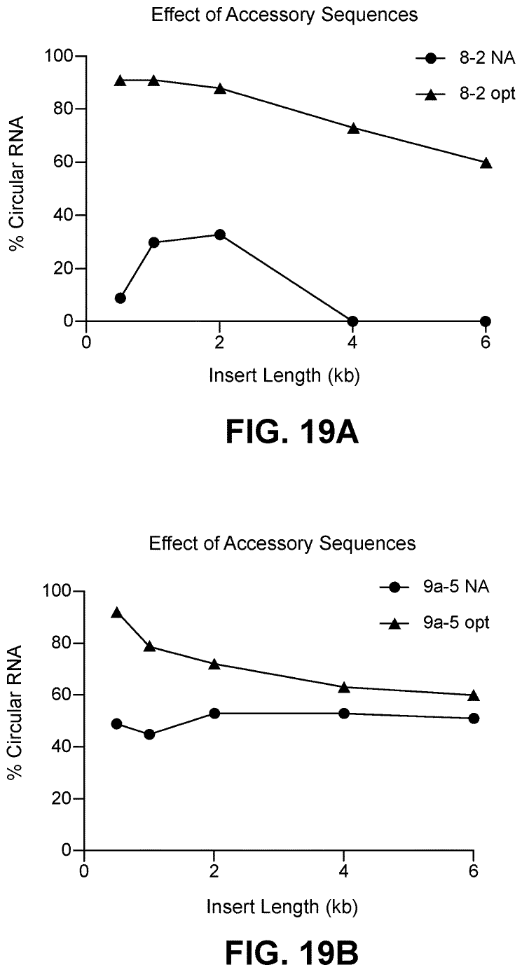

Various accessory elements were identified to determine their effect on the Group I or Group II introns. Preliminary analysis of the presence of accessory element (e.g., internal or external spacers and/or homology arms) were tested using SEC-HPLC analysis of post IVT reaction of DNA templates comprising the Groups I or Group II intron and either with or without the presence of accessory elements. The collected results across two Anabaena permutation sites can be seen in FIGS. 19a and 19b.

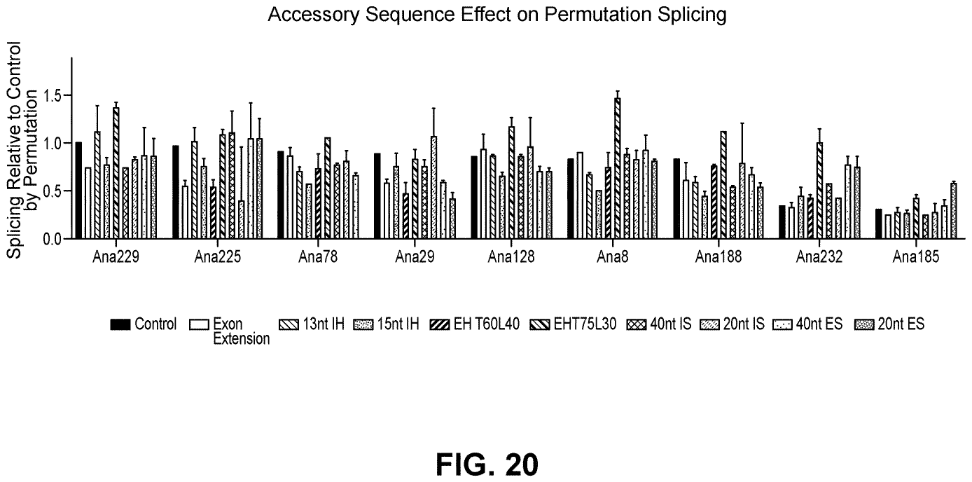

Further analysis of the effect on accessory elements was conducted on DNA templates comprising, in the following 5′ to 3 order: a T7 polymerase promoter, a 3′ Anabaena intron and exon, a CVB3 IRES, a firefly luciferase coding element, a stop codon, a 5′ Anabaena intron and exon, and an extended XbaI. One or more accessory elements (e.g., internal spacer, external spacer, internal homology arm, external homology arm) with different lengths were added to each DNA template to determine the effect of adding said accessory element. Control DNA templates were designed in the following 5′ to 3′ order: a T7 polymerase promoter sequence, an AC spacer sequence, 3′ Anabaena intron and exon sequences, a 5′ internal homology arm sequence, an AC spacer sequence, a CVB3 IRES sequence, a firefly luciferase coding element, an AC spacer sequence, a 3′ internal homology arm sequence, 5′ Anabaena exon and intron, sequences, and an AC spacer sequence. DNA templates were comprised of a sequence from SEQ ID NOS: 3484-3571. An IVT reaction and SEC-HPLC analysis was conducted according to Example 1 for each of these DNA templates. Results from the DNA templates are shown in FIG. 20.

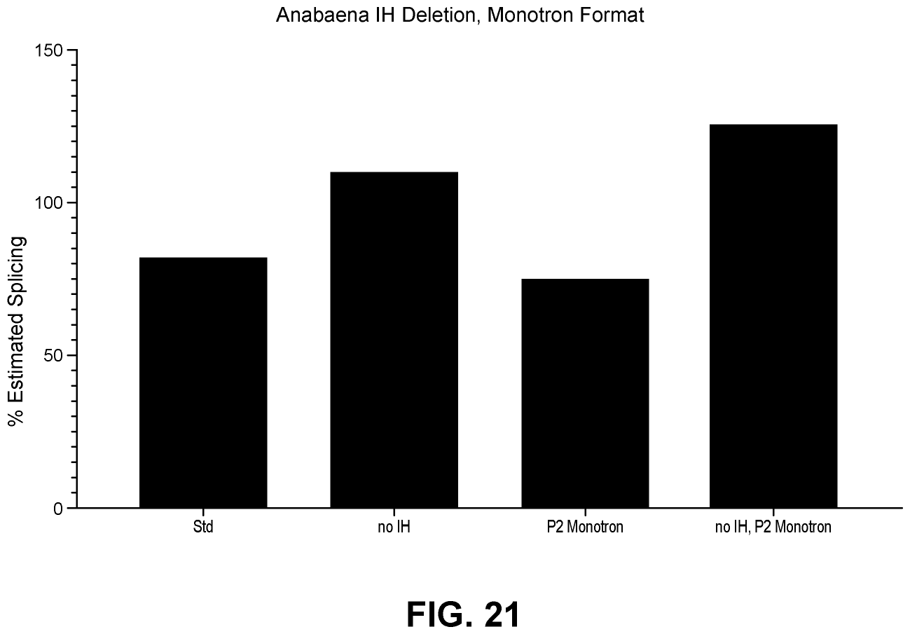

Effect of removal of internal homology arms was evaluated by assessing percent estimated splicing for DNA templates comprising an Anabaena monotron and terminal element. A standard non-monotron DNA template comprising two Anabaena permuted intron-exon elements was used as the control. The control DNA templates as well as the DNA templates comprising a 3′ monotron element were either given internal homology arms or were designed without the homology arms. DNA templates were comprised of a sequence from SEQ ID NOS: 3628 and 3633-3634. All of the DNA templates were allowed to undergo an IVT reaction, and the resulting solutions were analyzed using SEC-HPLC techniques provided in Example 1. Estimated percent splicing of the DNA templates are shown in FIG. 21.

### Example 3: Refolding

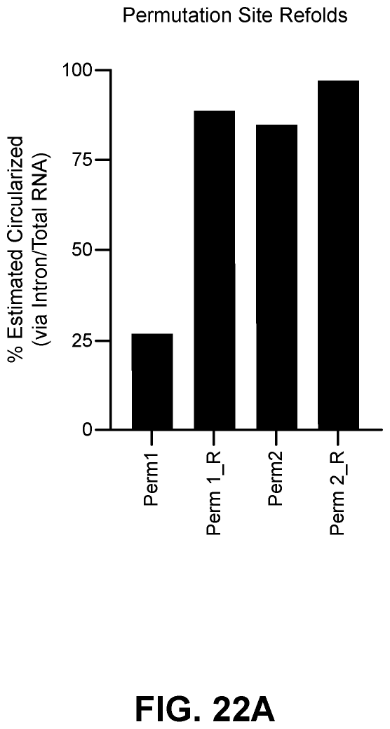

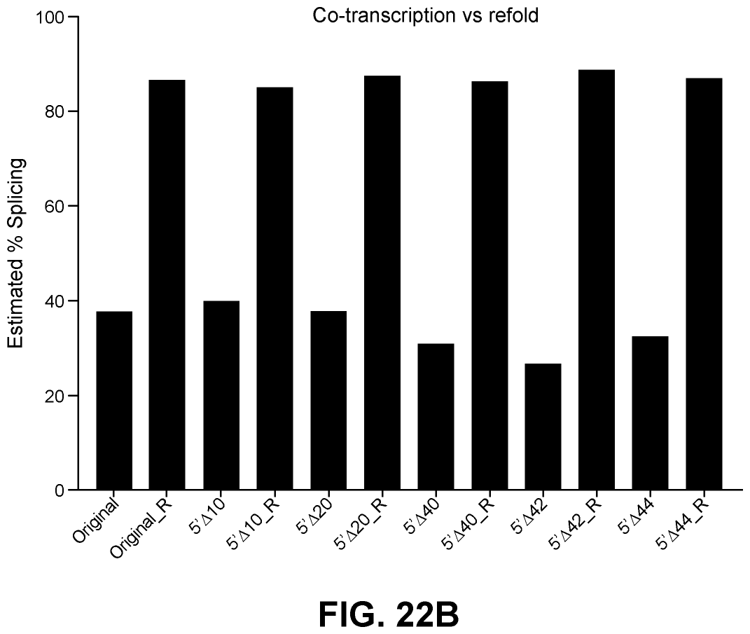

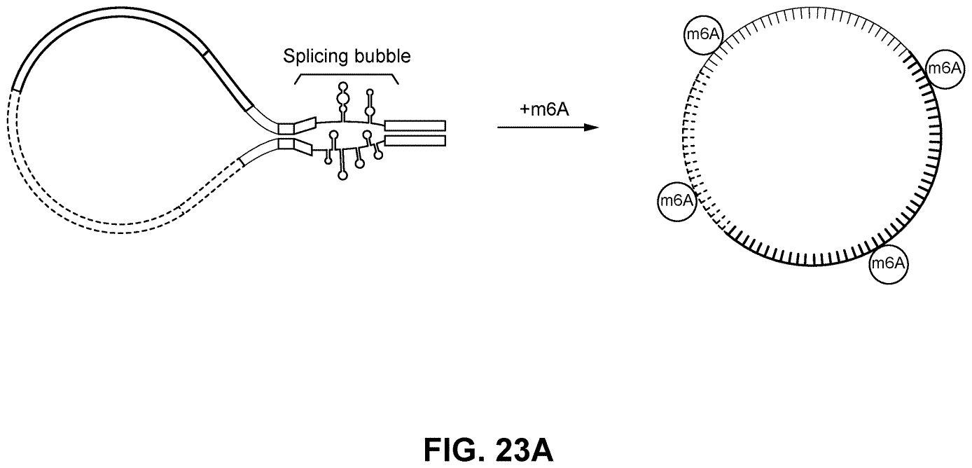

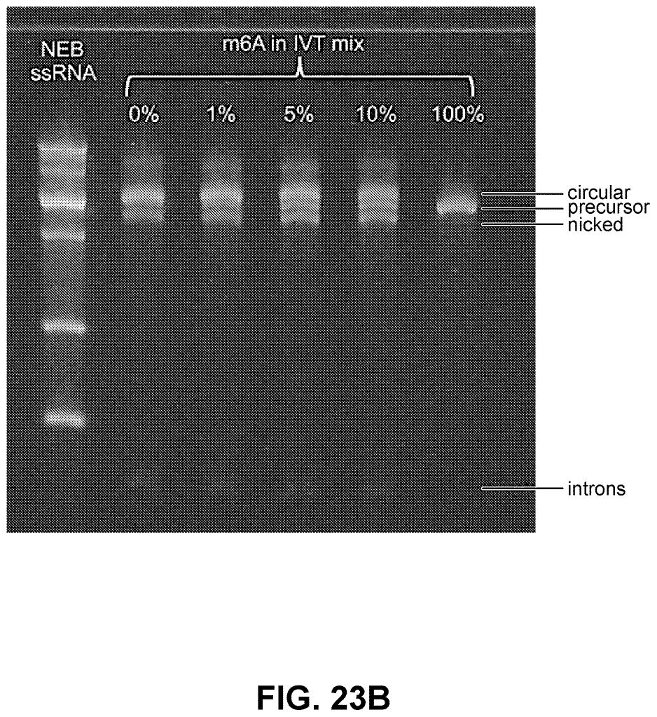

Preliminary testing for oRNA precursor RNA sequences with two different intron permutation sites were either allowed to circularize co-transcriptionally or circularized co-transcriptionally and then refold by exchanging the IVT buffer components with water. The RNAs were heated and cooled. A commercially available T4 RNA ligase I buffer, and guanosine nucleotide were added to the RNA solution and then further heated to 55° C. for 8 minutes. The results of the precursor RNA sequence for two different intron permutation sites are shown in FIG. 22a. A panel of 3′ exon fragment minimization RNA constructs comprising Anabaena introns and exons were tested under similar conditions to the preliminary refolding experiment with the two different intron permutation sites. The results of the panel of 3′ exon fragments are shown in FIG. 22b.

### Example 4: Incorporation of Modifications

Linear mRNA or circular RNA precursors were synthesized by runoff in vitro transcription (IVT) from a linearized plasmid DNA template comprising, in the following 5′ to 3′ order: T7 promoter, a 3′ intron segment, a 3′ exon segment, an internal ribosome entry site (IRES), a coding region (e.g., firefly luciferase), a 5′ exon segment, and a 3′ intron segment, and a XbaI restriction site. 3′ and 5′ introns were permuted from an Anabaena intron. The IRES used in the DNA template comprised a CVB3 IRES.

To obtain RNA bearing nucleotide modifications, the transcription reaction was assembled with the replacement of one (or more) of the basic nucleoside triphosphates (NTPs) with the corresponding triphosphate-derivative(s) of the modified nucleotide m6A. In each transcription reaction, all four nucleotides or their derivatives were present in concentrations shown in the table below (i.e., each of the reaction mixes comprise a ratio NTP to m6A ratio in the table below). To obtain RNA containing increasing amounts of m6A, the transcription reactions were performed in a reaction mix in which the ratio of modified ATP relative to the unmodified ATP was 0%, 1%, 5%, 10%, and 100%. 7.5× of a commercial reaction buffer along with GMP and T7 RNA polymerase was present in the transcription reaction solution.

Post IVT reaction, 5 μL of DNAse enzyme was added to digest remaining DNA templates. Each of the reaction mixtures were purified using a commercially available RNA column purification method. 5 μL of the resulting solution was injected onto SEC-HPLC. FIG. 1B shows a gel with the resulting IVT solutions. Percent estimated circularization was calculated from the SEC-HPLC analysis based on the resulting circular RNA peaks and is shown in the table below.

| Percent m6A in | | | Percent Estimated |
|---|---|---|---|
| IVT | NTP:m6A | NTP:m6A Ratio | Circularization |
| 0% m6A | ATP:m6A | 5.94:0.06 | 63.60% |
| | CTP:m6A | 6:0.06 | |
| | GTP:m6A | 6:0.06 | |
| | UTP:m6A | 6:0.06 | |
| 1% m6A | ATP:m6A | 5.94:0.06 | 64.10% |
| | CTP:m6A | 6:0.06 | |
| | GTP:m6A | 6:0.06 | |
| | UTP:m6A | 6:0.06 | |
| 5% m6A | ATP:m6A | 5.7:0.06 | 44.70% |
| | CTP:m6A | 6:0.06 | |
| | GTP:m6A | 6:0.06 | |
| | UTP:m6A | 6:0.06 | |
| 10% m6A | ATP:m6A | 5.4:0.06 | 39.90% |
| | CTP:m6A | 6:0.06 | |
| | GTP:m6A | 6:0.06 | |
| | UTP:m6A | 6:0.06 | |
| 100% m6A | ATP:m6A | 0.00 | 0.00% |
| | CTP:m6A | 0.00 | |
| | GTP:m6A | 0.00 | |
| | UTP:m6A | 0.00 | |

In some embodiments, the DNA templates described herein are designed to comprise a T4 td or Azoarcus derived intron; an Hunnivirus derived IRES, and/or a wasabi fluorescent protein or Axicabtagene ciloleucel chimeric antigen receptor (CAR) coding region. To obtain RNA containing increasing amounts of T, m5C, or another modification described herein, the transcription reaction can be performed in a reaction mix in which the ratio of one particular NTP is relative to the corresponding unmodified NTP is 0%, 1%, 5%, 10%, and 100%.

Similar to the DNA templates comprising PIE segments, DNA templates capable of forming circular RNAs using ligase (i.e., via splint ligation) or other enzymatic methods are designed to incorporate modifications. Precursor RNA formed from the DNA templates post IVT are created with optimized splint oligonucleotides designed to bring the ends of the precursor RNA into proximity for ligation. Linear precursors for splint-mediated ligation are designed to have all the same sequence features as the PIE-linear RNA counterparts except for the addition of short adaptor sequences onto the 5′ and 3′ ends of the precursor RNA instead of a PIE segments (e.g., exemplary DNA splint 5′-GTTTGTGGTTCGTGCGTCTCC GTGCTGTTCTGTTGGTGTGGG-3′). Splint ligation precursors are synthesized as described previously, except a 10-fold excess of GMP is added to the IVT transcription reactions. 25 μg purified precursor RNA is heated to 70° C. for 5 minutes in the presence of the DNA splint at a concentration of 5 μM in a 90 μL reaction. The reaction is allowed to cool to room temperature, and then a commercially available T4 RNA Ligase I Buffer (e.g., NEB) is added to a final concentration of 1×. NTP and m6A amounts are added according to the concentrations listed in the table below. 50U of commercially available T4 RNA Ligase I (e.g., NEB) are added to the reaction mixture. Reactions are incubated at 37° C. for 30 minutes and then column purified. 5 μL of the resulting solution is injected onto SEC-HPLC and analyzed for percent estimated circularization.

### Example 5: Protecting Certain Regions of the Linear Precursor Template from Modifications

a. General Construct Design

Two separate strands of linear precursor RNA each comprising intron segments and exon segments are designed. One of the two strands of the linear precursor RNA (i.e., Strand 1) comprises non-modified elements throughout its sequence. The second of the two strands of linear precursor RNA (i.e., Strand 2) comprises at least one modified nucleotide (e.g., m6A, T, and/or m5C). Strand 1 and Strand 2 comprise exon and intron in a non-self-circularizing orientation (e.g., each strand comprises in the following order an exon, intron, exon) but have exon and introns with complementarity towards each other. Exemplary depictions of the two linear precursor strands are provided in FIGS. 24B-24D. Strand 2 is created with modifications using methods described in Example 4. Placement of the modifications along the linear precursor RNA for Strand 2 occurred simultaneously to the IVT reaction, wherein m6ATP was incorporated into the linear precursor RNA solutions during the IVT reaction) post in modification incorporation into solution during the IVT reaction of the DNA template. Strand 1 and Strand 2 may have intron and exon segments derived from naturally occurring DNA plasmids comprising Group I or Group II introns illustrated in FIG. 24A or a synthetic variation. Strand 1 is designed with an internal ribosome entry site (IRES). Strand 1 and Strand 2 are designed to comprise parts of a coding sequence of a gene of interest (e.g., firefly luciferase protein, Wasabi fluorescent protein, or Axicabtagene ciloleucel). Both parts of the coding sequence are designed to have complementarity to an exon. The coding sequence is scanned for regions that are homologous to allow splicing to occur, e.g., without altering the resulting coding sequence in a final circular construct. Additional elements are added to one or more of the strands (e.g., spacers and/or internal homology sequences) to promote circularization.

Each of the strands are separately produced from the DNA templates using IVT reactions with a Mg²⁺ concentration of either 7 mM or 34 mM (exemplary Mg²⁺ concentrations depicted in the table below). After the synthesis of both strands, Strand 1 and Strand 2 are purified and incubated together in a solution containing Mg²⁺ as well as guanosine-monophosphate to facilitate trans-splicing between both strands and circularization (depicted in FIG. 24B3-FIG. 24D). The circular RNA product is then isolated, and column purified.

| | | Synthesis Details |
|---|---|---|
| Method 1 | Strand 1: construct comprises non-modified elements | Strand 1 and Strand 2 |
| | including: two distinct intron elements (e.g., 3′ | each independently |
| | permutation of the Intron 1 Element & 5′ permutation | undergo an IVT with |
| | of the Intron 2 Element), an IRES, and 2 corresponding | Mg 2+ concentrations of |
| | exon elements (e.g., Exon 1B & Exon 2A). Exon 2A is | 34 mM. Each strand is |
| | a part of a coding sequence of the gene of interest. | independently purified. |
| | Strand 2: construct comprises corresponding intron | Both strands are |
| | elements to Strand 1 (e.g., 5′ Permutation of the Intron | combined and incubated |
| | 2 Element & 3′ Permutation of the Intron 1 Element) as | in the presence of Mg2+ |
| | well as the corresponding Exon 2B and Exon 1A. Exon | and guanosine and then |
| | 2B is a part of a coding sequence of the gene of interest. | purified. |
| Method 2 | Strand 1: construct comprises non-modified elements | Strand 1 and Strand 2 |
| | including: two distinct intron elements (e.g., 3′ | each independently |
| | permutation of the Intron 1 Element & 5′ permutation | undergo an IVT with |
| | of the Intron 2 Element), the IRES, as well as 2 | Mg 2+ concentrations of |
| | corresponding exon elements (e.g., Exon 1B & Exon | 7 mM and 34 mM |
| | 2A). Exon 2A is a part of a coding sequence of the gene | respectively. Each |
| | of interest. | strand is independently |
| | Strand 2: construct comprises corresponding intron | purified. Both strands |
| | elements to Strand 1 (e.g., 5′ Permutation of the Intron | are combined and |
| | 2 Element & 3′ Permutation of the Intron 1 Element) as | incubated in the |
| | well as the corresponding Exon 2B and Exon 1A. Exon | presence of Mg2+ and |
| | 2B is a part of a coding sequence of the gene of interest. | guanosine and then |
| | | purified. |
| Method 3 | Strand 1: construct comprises non-modified elements | Strand 1 and Strand 2 |
| | including: two distinct intron elements (e.g., 3′ | each independently |
| | permutation of the Intron 1 Element & 5′ permutation | undergo an IVT with |
| | of the Intron 2 Element), the IRES, as well as 2 | Mg 2+ of concentrations |
| | corresponding exon elements (e.g., Exon 1B & Exon | 7 mM. Each strand is |
| | 2A). Exon 2A is a part of a coding sequence of the gene | independently purified. |
| | of interest. | Both strands are |
| | Strand 2: construct comprises corresponding intron | combined and incubated |
| | elements to Strand 1 (e.g., 5′ Permutation of the Intron | in the presence of Mg2+ |
| | 2 Element & 3′ Permutation of the Intron 1 Element) as | and guanosine and then |
| | well as the corresponding Exon 2B and Exon 1A. Exon | purified. |
| | 2B is a part of a coding sequence of the gene of interest. | |
| Method 4 | Strand 1: construct comprises non-modified elements | Strand 1 independently |
| | including: two distinct intron elements (e.g., 3′ | undergoes an IVT with |
| | permutation of the Intron 1 Element & 5′ permutation | Mg 2+ concentration of |
| | of the Intron 2 Element), the IRES, as well as 2 | 7 mM. Strand 1 is |
| | corresponding exon elements (e.g., Exon 1B & Exon | independently purified. |
| | 2A). Exon 2A is a part of a coding sequence of the gene | Strand 1 and Strand 2 |
| | of interest. | are combined and |
| | | undergo an IVT reaction |
| | | with a Mg2+ |
| | | concentration of 34 mM |
| | | and guanosine then |
| | | purified. |

b. Exemplary Precursor RNA Strands

Various iterations of the two linear precursors can form circular RNAs with modifications and are tested for percent estimated circularization. FIGS. 24B-24D illustrate three exemplary linear RNA precursor combinations, e.g., Strand 1 and Strand 2 each comprising two introns and exons; Strand 1 and Strand 2 each comprising a monotron segment and two exon segments; and Strand 1 comprising an intron and two exon segments and Strand 2 comprising a monotron intron and a (non-monotron) intron segment along with two exon segments. Other variations that are tested include Strand 1 comprising two monotron introns along with two exons and Strand 2 comprising two exon segments.

### Example 6: Immunogenicity of Modified Circular RNAs

To assess immunogenicity, the circular RNAs are tested in a cytokine release assay and cell viability assay after transfection of A549 cells. The circular RNAs comprise 5′ and 3′ exons, internal ribosome entry site (IRES) and expression sequence encoding firefly luciferase or a chimeric antigen receptor (CAR). Cytokine release is measured 24 h after transfection. Cell viability is measured 36 h after transfection. RIG-I and IFN-beta 1 transcript induction are measured 18 h after transfection. Briefly, A549 cells are cultured at 37° C. and 5% CO2 in Dulbecco's Modified Eagle's Medium (4500 mg/L glucose) supplemented with 10% heat inactivated FBS and penicillin/streptomycin. 200 ng RNA is transfected into 10,000 cells/100 uL using lipofectamine.

### Example 7: In Vitro Transcription of Circular RNAs in Exemplary Magnesium Concentrations

a. DNA Template Comprising Azoarcus Intron

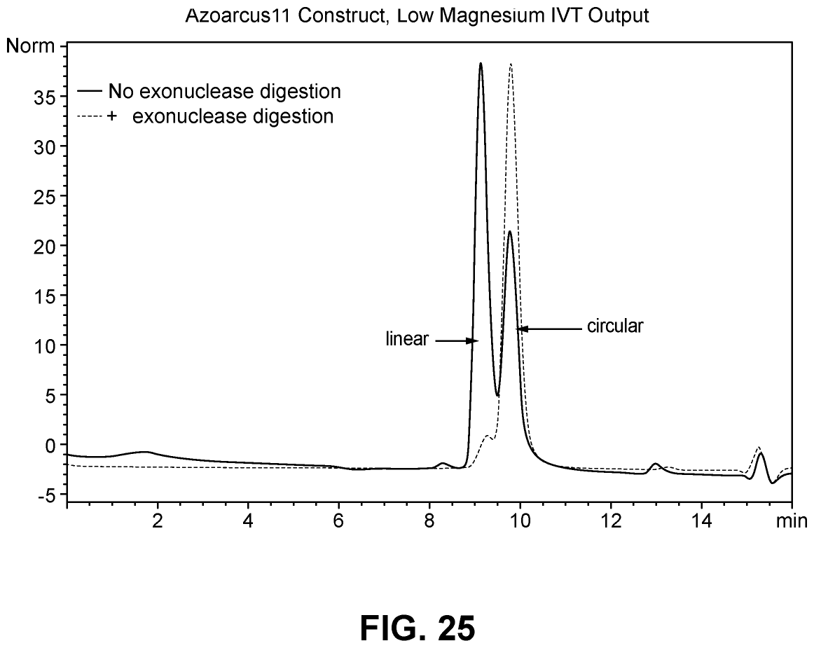

A DNA template was designed comprising (1) a T7 polymerase promoter, (2) 5′ and 3′ intron segments, (4) 5′ and 3′ exon segments, (5) a Coxsackievirus B3 (CVB3) internal ribosome entry site (IRES), (6) an expression sequence encoding firefly luciferase (fLuc), (7) and a XbaI restriction site. 5′ and 3′ intron and/or exon segments were designed using a naturally occurring Azoarcus intron and exon sequences. The naturally occurring Azoarcus intron sequences were permuted at position 11 in FIG. 8B. The DNA template was then linearized using a XbaI restriction enzyme and in vitro transcribed in a 34 mM magnesium concentration or 12.75 mM magnesium concentration. The resulting IVT product was purified using a commercially available silica column purification method. Post purification, the IVT products were analyzed by size exclusion high performance liquid chromatography (SEC-HPLC) in a 1×-tris-EDTA running buffer (e.g., from Agilent) at a pH of 6 to determine splicing activity. Circularization of the IVT products was confirmed using exonuclease digestion of linear RNAs in the IVT product solution. IVT products were analyzed again by size exclusion high performance liquid chromatography (SEC-HPLC) in a 1×-tris-EDTA running buffer (e.g., from Agilent) at a pH of 6 to analyze circularization in a low magnesium environment. SEC-HPLC results are shown in FIG. 25.

b. DNA Template Comprising Azoarcus or Anabaena Intron

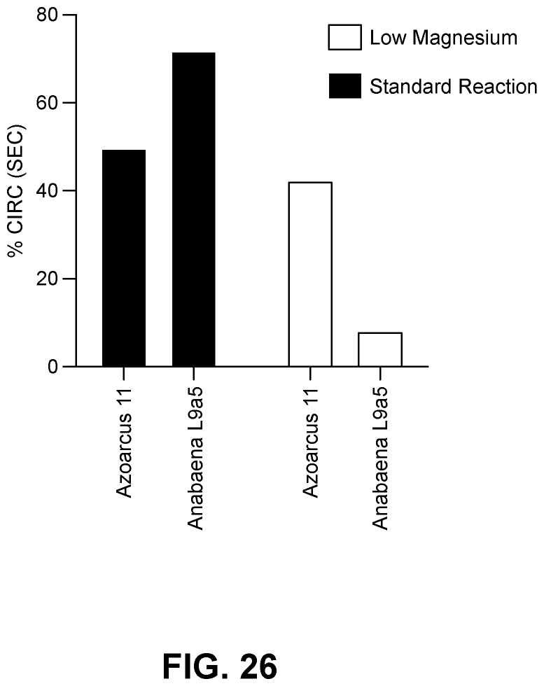

Two DNA templates were designed to comprise (1) a T7 polymerase promoter, (2) 5′ and 3′ intron segments, (4) 5′ and 3′ exon segments, (5) a Coxsackievirus B3 (CVB3) or Caprine kobuvirus internal ribosome entry site (IRES), (6) an expression sequence encoding firefly luciferase (fLuc), (7) and a XbaI restriction site. 5′ and 3′ intron and/or exon segments were designed using a naturally occurring Azoarcus or Anabaena intron and exon sequences. The naturally occurring Anabaena intron sequences were permuted at position 230 in FIG. 8A to form the 5′ and 3′ intron segments. The naturally occurring Azoarcus intron sequences were permuted at position 11 in FIG. 8B to form the 5′ and 3′ intron segments. DNA template 1 comprised an Anabaena intron segment and a Caprine kobuvirus IRES. DNA template 2 comprised an Azoarcus intron segment and a CVB3 IRES. The DNA template was then linearized using an XbaI restriction enzyme and in vitro transcribed in 34 mM magnesium concentration. The resulting IVT product was purified using a commercially available silica column purification method. Post purification, the IVT products were analyzed by size exclusion high performance liquid chromatography (SEC-HPLC) in a 1×-tris-EDTA running buffer (e.g., from Agilent) at a pH of 6 to determine splicing activity. IVT reactions were then repeated for the DNA templates under 12.75 mM magnesium concentration. Circularization of the IVT products was confirmed using exonuclease digestion of linear RNAs in the IVT product solution. IVT products were analyzed again by size exclusion high performance liquid chromatography (SEC-HPLC) in a 1× tris-EDTA running buffer (e.g., from Agilent) at a pH of 6 to analyze circularization in a low magnesium environment. Circular RNA IVT product yield was quantified using spectrophotometry and provided in the table below. Estimated percent circularization is shown in FIG. 26.

| | Concentration of Mg²⁺ | IVT Yield |
|---|---|---|
| DNA template 1 | 34 mM | 6.7 μg/μL |
| | 12.75 mM | 6.7 μg/μL |
| DNA template 2 | 34 mM | 6.8 μg/μL |
| | 12.75 mM | 6.5 μg/μL |

### Example 8: Group I Permutations for Tetrahymena thermophila, T4 td, Staphylococcus Phage Twort and Coxiella Burnetii

a. Tetrahymena thermophila Introns

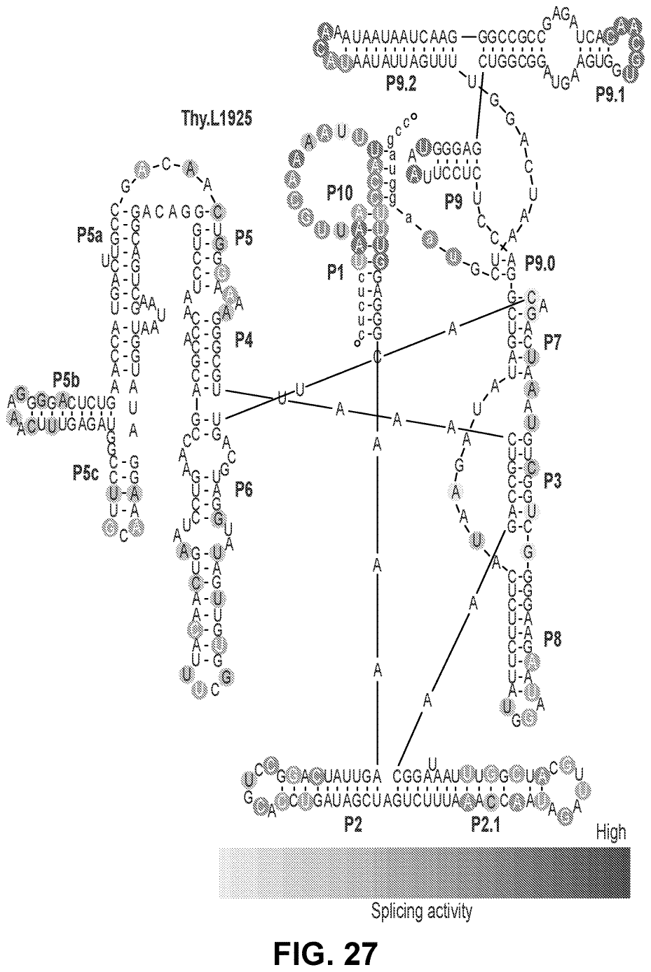

Plasmid DNA containing Tetrahymena thermophila introns disclosed herein capable of splicing in a native context as described in Example 1a (above) were permuted in various different locations. Permutation locations were selected using a GI intron database or RNAFold prediction to minimize impact of permutation to overall structure and functionality of the intron. The permuted intron sequences and exon segment sequences (e.g., exons segments extracted from the original plasmid DNA with at least 15 nucleotides from the 5′ and 3′ end) were collected to form 5′ and 3′ permuted intron and exon (PIE) segments. A T7 RNA polymerase was added before the PIE construct, and a XbaI restriction site was added after the PIE construct to form a DNA template (e.g., for each of the permutation sites, IVT reactions were performed on DNA templates comprising (1) a T7 polymerase promoter, (2) 5′ and 3′ permuted intron segments formed from the permuted site(s), (3) 5′ and 3′ exon segments, and (4) an XbaI restriction site). IVT reactions were performed on the DNA templates and resulting solutions were purified via silica column purification. Purified IVTs products were analyzed by fragment analyzer or SEC-HPLC in 1× tris-EDTA running buffer (Agilent), pH6, to determine splicing activity. FIG. 27 provides a heat map with the splicing activity of the various permuted sites tested along the Tetrahymena thermophila Group I intron. Each location in FIG. 27 corresponds with a permuted intron sequence (SEQ ID NO: 25573) that was used to create permuted intron exon sequences for the DNA templates.

b. T4 td Introns

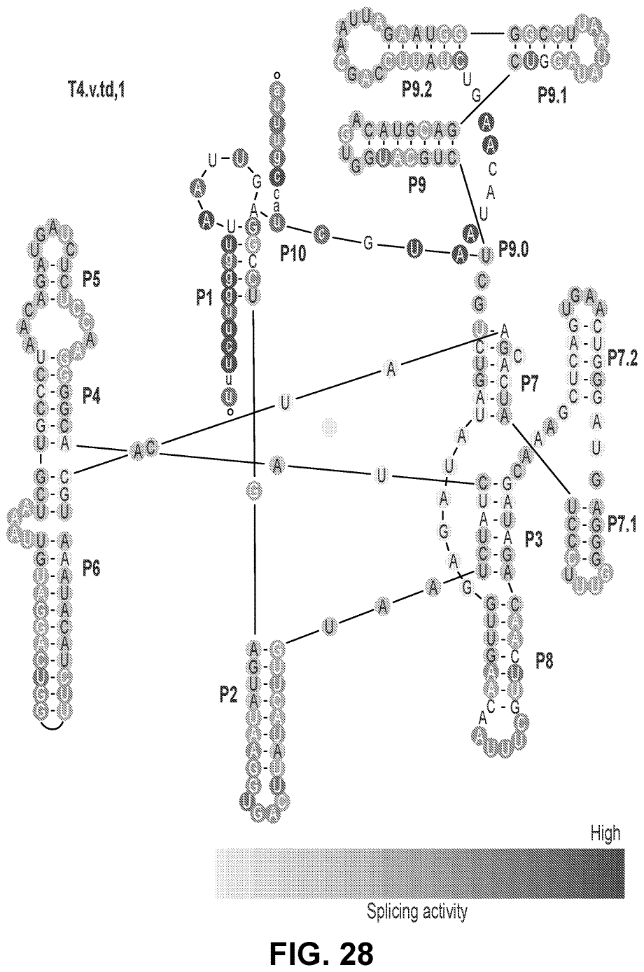

Plasmid DNA containing T4 td introns disclosed herein capable of splicing in a native context as described in Example 1a (above) were permuted in various different locations. Permutation locations were selected using GI intron database or RNAFold prediction to minimize impact of permutation to overall structure and functionality of the intron. The permuted intron sequences and exon segment sequences (e.g., exons segments extracted from the original plasmid DNA with at least 15 nucleotides from the 5′ and 3′ end) were collected to form 5′ and 3′ permuted intron and exon (PIE) segments. A T7 RNA polymerase was added before the PIE construct, and a XbaI restriction site was added after the PIE construct to form a DNA template (e.g., for each of the permutation sites, IVT reactions were performed on DNA templates comprising (1) a T7 polymerase promoter, (2) 5′ and 3′ permuted intron segments formed from the permuted site(s), (3) 5′ and 3′ exon segments, and (4) an XbaI restriction site). IVT reactions were performed on the DNA templates and resulting solutions were purified via silica column purification. Purified IVTs products were analyzed by fragment analyzer or SEC-HPLC in 1× tris-EDTA running buffer (Agilent), pH6, to determine splicing activity. FIG. 28 provides a heat map with the splicing activity of the various permuted sites tested along the T4 td Group I intron. Each location in FIG. 28 corresponds with a permuted intron sequence (SEQ ID NO: 25574) that was used to create permuted intron exon sequences for the DNA templates.

c. Staphylococcus Phage Twort Introns

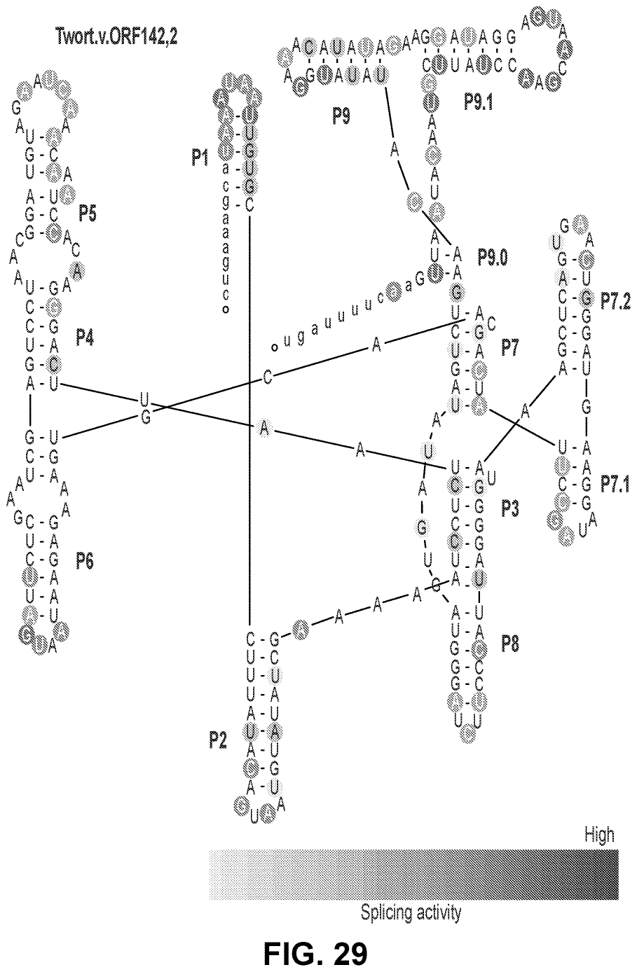

Plasmid DNA containing Staphylococcus phage Twort introns disclosed herein capable of splicing in a native context as described in Example 1a (above) were permuted in various different locations. Permutation locations were selected using GI intron database or RNAFold prediction to minimize impact of permutation to overall structure and functionality of the intron. The permuted intron sequences and exon segment sequences (e.g., exons segments extracted from the original plasmid DNA with at least 15 nucleotides from the 5′ and 3′ end) were collected to form 5′ and 3′ permuted intron and exon (PIE) segments. A T7 RNA polymerase was added before the PIE construct, and a XbaI restriction site was added after the PIE construct to form a DNA template (e.g., for each of the permutation sites, IVT reactions were performed on DNA templates comprising (1) a T7 polymerase promoter, (2) 5′ and 3′ permuted intron segments formed from the permuted site(s), (3) 5′ and 3′ exon segments, and (4) an XbaI restriction site). IVT reactions were performed on the DNA templates and resulting solutions were purified via silica column purification. Purified IVTs products were analyzed by fragment analyzer or SEC-HPLC in 1× tris-EDTA running buffer (Agilent), pH6, to determine splicing activity. FIG. 29 provides a heat map with the splicing activity of the various permuted sites tested along the Staphylococcus phage Twort Group I intron. Each location in FIG. 29 corresponds with a permuted intron sequence (SEQ ID NO:3006) that was used to create permuted intron exon sequences for the DNA templates.

d. Coxiella burnetii Introns

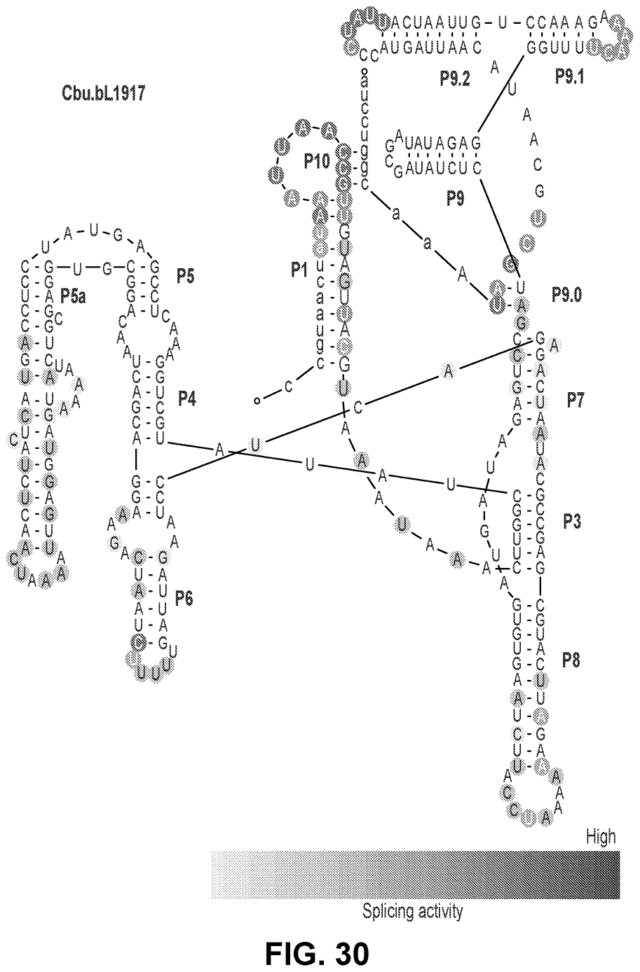

Plasmid DNA containing Coxiella burnetii introns disclosed hereincapable of splicing in a native context as described in Example 1a (above) were permuted in various different locations. Permutation locations were selected using GI intron database or RNAFold prediction to minimize impact of permutation to overall structure and functionality of the intron. The permuted intron sequences and exon segment sequences (e.g., exons segments extracted from the original plasmid DNA with at least 15 nucleotides from the 5′ and 3′ end) were collected to form 5′ and 3′ permuted intron and exon (PIE) segments. A T7 RNA polymerase was added before the PIE construct, and a XbaI restriction site was added after the PIE construct to form a DNA template (e.g., for each of the permutation sites, IVT reactions were performed on DNA templates comprising (1) a T7 polymerase promoter, (2) 5′ and 3′ permuted intron segments formed from the permuted site(s), (3) 5′ and 3′ exon segments, and (4) an XbaI restriction site). IVT reactions were performed on the DNA templates and resulting solutions were purified via silica column purification. Purified IVTs products were analyzed by fragment analyzer or SEC-HPLC in 1× tris-EDTA running buffer (Agilent), pH6, to determine splicing activity. FIG. 30 provides a heat map with the splicing activity of the various permuted sites tested along the Coxiella burnetii Group I intron. Each location in FIG. 30 corresponds with a permuted intron sequence (SEQ ID NO:2997) that was used to create permuted intron exon sequences for the DNA templates.

### Example 9: Incorporating Base Modifications into Circular RNAs

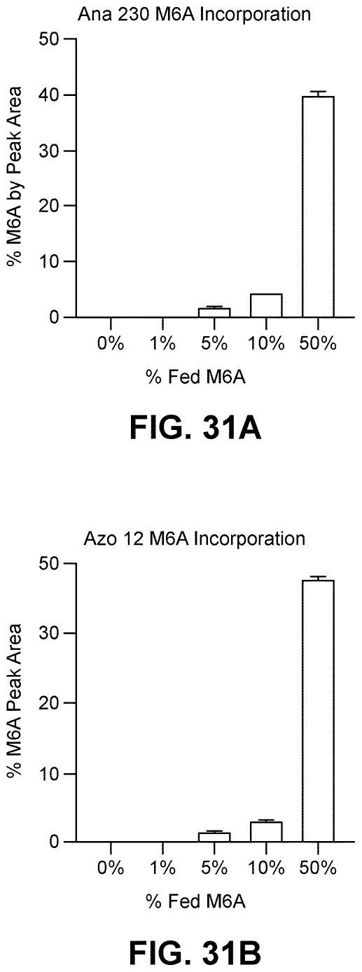

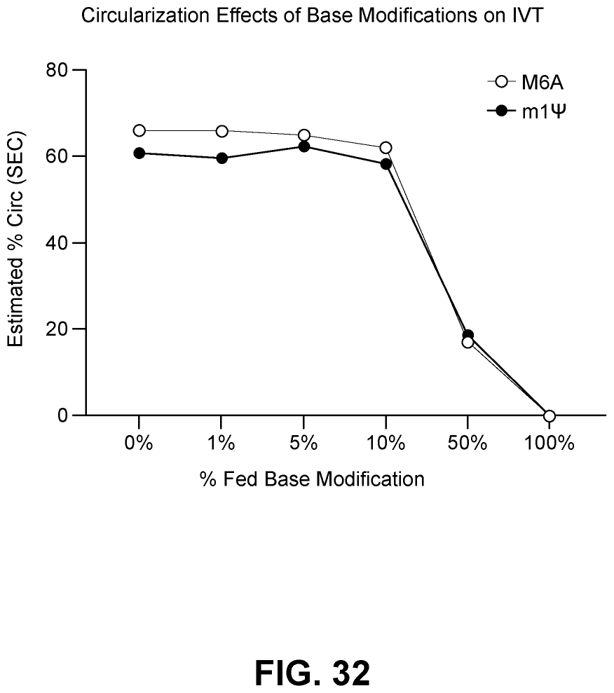

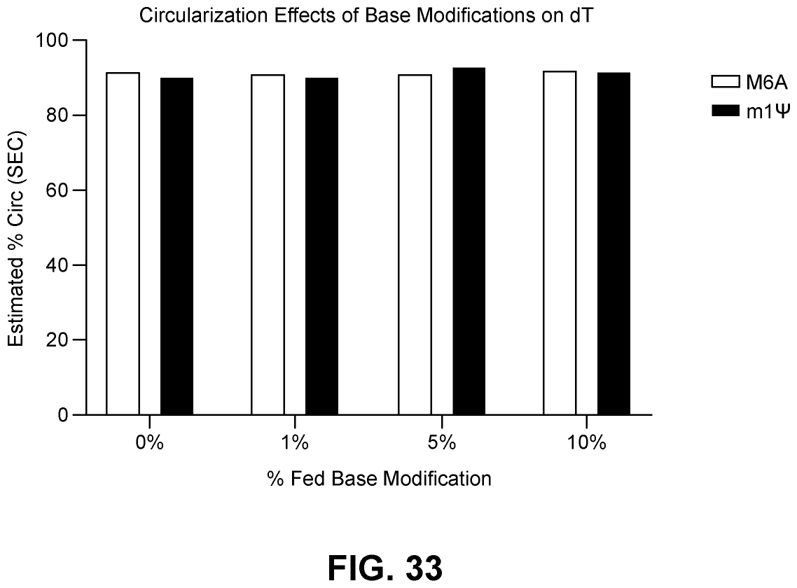

IVT reactions were performed on DNA templates comprising either an Anabaena or Azoarcus intron. Each IVT reaction contained an ascending concentration of a modified nucleotide triphosphate (e.g., m6A-TP or m1Ψ-TP) and a descending concentration of the corresponding non-modified nucleotide triphosphate (e.g., ATP, CTP, GTP, or UTP). For example, if 5% m6A amount was used in the IVT reaction, 95% of the non-modified nucleotide triphosphate was also used. m6A modification incorporation was tested for 0%, 1%, 5%, 10%, 50% and 100% concentration of m6A-TP. Anabaena intron segments were developed from a naturally occurring Anabaena intron sequence comprised a position 230 permutation site in FIG. 8A. Azoarcus intron segments were developed from a naturally occurring Azoarcus intron sequence that comprised a position 12 permutation site in FIG. 8B. The RNA polynucleotide resulting from the IVT reaction was purified using purification methods using silica column purification. The amount of m6A incorporation into the RNA formed from the IVT reaction of the DNA templates were quantified by hydrolyzing and using phosphatase treatment and then observed for m6A peaks in a commercially available ion-pair reverse-phase chromatography (IPRP). Percentage of incorporation of m6A modification was calculated based on the area of the m6A modification peak as compared to the total number of bases (i.e., A, C, G, and T modified and non-modified nucleotides and/or nucleosides) are present in the DNA template. The table below provides the percentage of A, C, G, and U nucleotide and/or nucleosides present in the Anabaena and Azoarcus intron segments. FIGS. 31A and 31B provide the percent m6A incorporation into the DNA templates comprising the Anabaena intron segments (FIG. 31A) or Azoarcus intron segments (FIG. 31B). Purified IVT products were analyzed by SEC-HPLC in 1× tris-EDTA running buffer (e.g., from Agilent) at a pH of 6 to determine splicing activity and estimated percent circularization. Estimated percent circularization is shown in FIG. 32. As depicted in FIG. 32, percent circularization decreases at a 10% or greater concentration of m6A-TP or m1Ψ-TP into the IVT reaction. In some embodiments, the IVT products were purified using oligo-dT purification. The estimated percent circularization results of the IVT products purified by oligo-dT purification is illustrated for 0%, 1%, 5% and 10% in FIG. 33. Greater than 10% m6A-TP or m1Ψ-TP was analyzed to have lower circularization efficiency, modified poly-A tails, and peaks shifts due to high modification leading to elution of oligo-dT t cause co-elution of linear and circular RNA polynucleotide IVT products and incomplete purification.

### Example 10: Circular RNA Comprising Modifications

a. Bioluminescence and IFNβ secretion

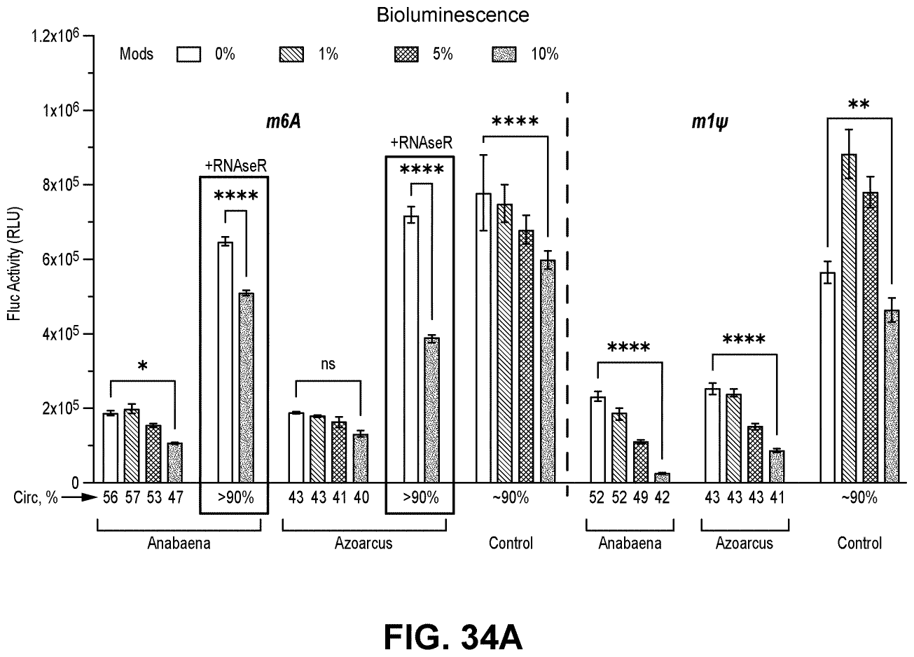

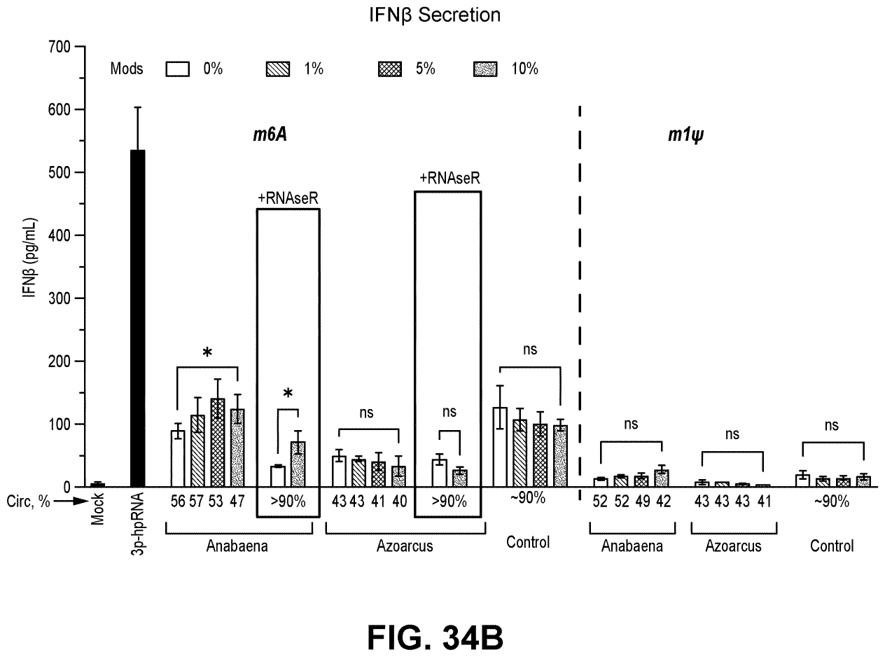

IVT reactions were performed on three different DNA templates comprising: (1) a T7 polymerase promoter, (2) 5′ and 3′ permuted intron segments, (3) 5′ and 3′ intron segments, (4) either a CVB3 or Caprine kobuvirus IRES, (5) an expression sequence encoding firefly luciferase, and (6) XbaI restriction site. Two of the three DNA templates (including the control DNA) comprised intron segments developed from naturally occurring Anabaena intron sequences that were permuted at position 230 in FIG. 8A. The third DNA template comprised intron segments developed from naturally occurring Azoarcus intron sequences that were permuted at position 12 in FIG. 8B. The control DNA template further comprised 5′ and 3′ internal spacers, 5′ and 3′ internal duplex region. Each of the IVT reaction was given m6A or m1Ψ modified nucleotides at a 0%, 1%, 5%, or 10%. The resulting IVT products (e.g., comprising circular RNAs) were purified via a silica column purification method and analyzed by SEC-HPLC in 1× tris-EDTA running buffer (e.g., from Agilent) at a pH of 6. M6A modified IVT reactions were further treated with RNase R endonuclease for the non-control constructs and purified using a silica column. The circular RNAs made from the control DNA were purified with oligo-dT and then a silica column. The resulting purified circular RNAs were transfected using a commercially available lipofectamine into A549 cells. For control, the A549 cells were either treated with purely lipofectamine (negative control) or 5′ triphosphate hairpin RNA (3p-hpRNA) (positive control). At 24 hours post transfection of the lipofectamine, the cells were lysed and measured for luminescence and analyzed using interferon-1-beta ELISA. The resulting luminescence and IFNβ data are depicted in FIG. 34A and FIG. 34B respectively.

b. Transfection Dosage

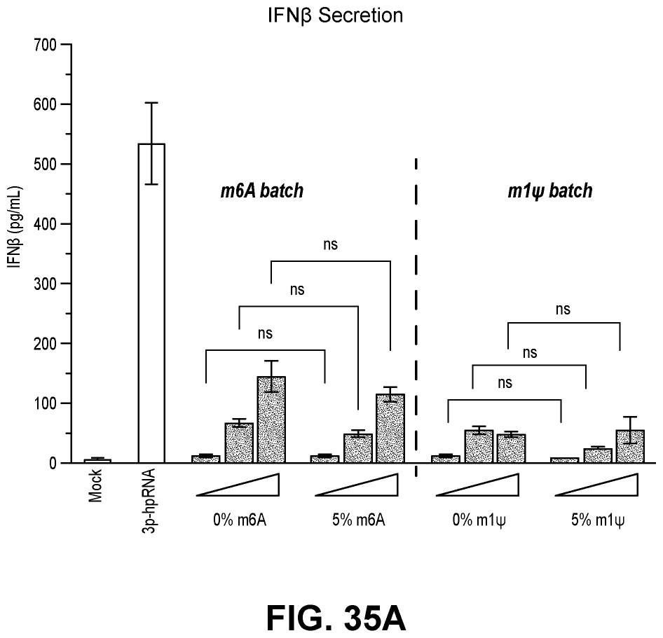

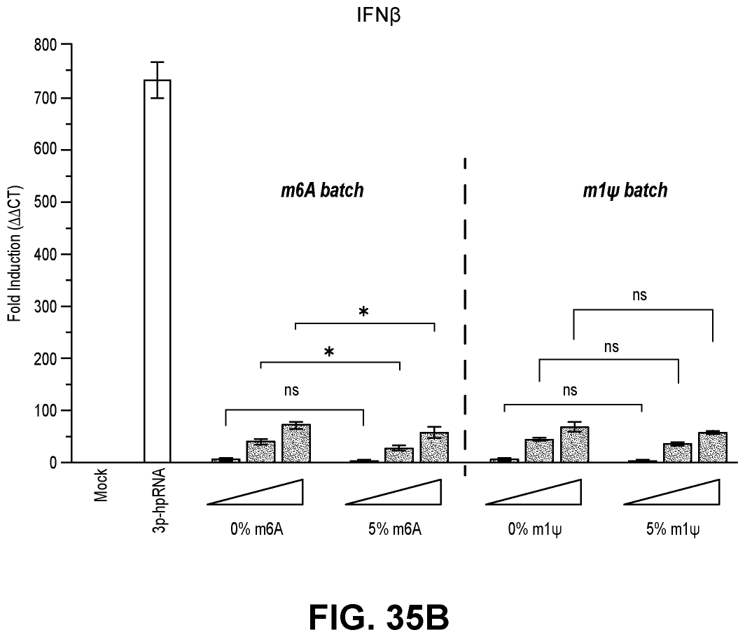

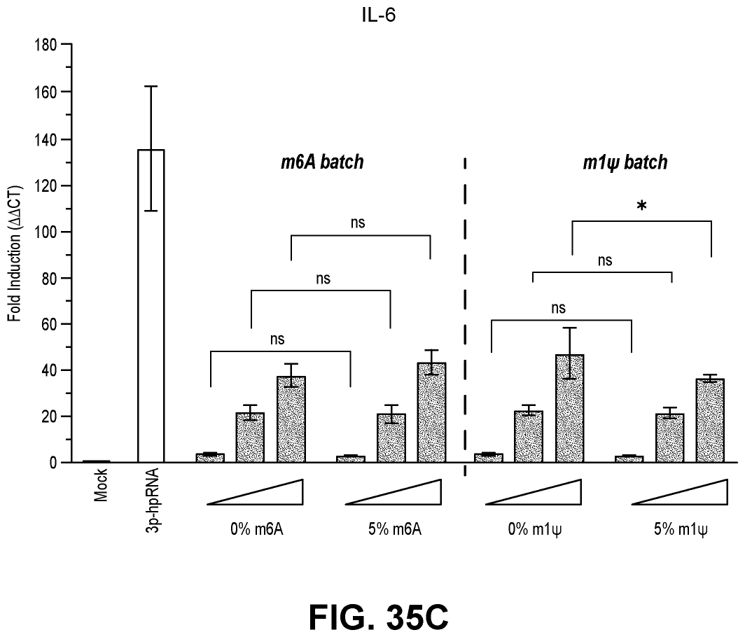

IVT reaction was performed on a DNA template comprising: (1) a T7 polymerase promoter, (2) 5′ and 3′ permuted intron segments, (3) 5′ and 3′ exon segments, (4) a Caprine kobuvirus IRES, (5) an expression sequence encoding firefly luciferase, and (6) a XbaI restriction site. Each of the IVT reactions were conducted with either no modifications (i.e., 0% m6A-TP or 0% m1Ψ-TP), 5% m6A-TP or 5% m1Ψ-TP. The resulting circular RNAs from the IVT reaction were purified using methods described herein. The purified circular RNAs were transfected into A549 cells using lipofectamine at either a low, medium or high dosage (i.e., 2×, 6×, 18× ng/well). Lipofectamine without an RNA polynucleotide was used as a negative control. 3′ triphosphate hairpin RNA transfected using lipofectamine was used as a positive control. After 24 hours, the IFNβ secretion levels were analyzed using interferon-1-beta ELISA (FIG. 35A), relative amounts of IFNβ (FIG. 35B) and IL-6 (FIG. 35C) secreted were analyzed using qPCR.

### Example 11: Effects of Modifications on Circularization

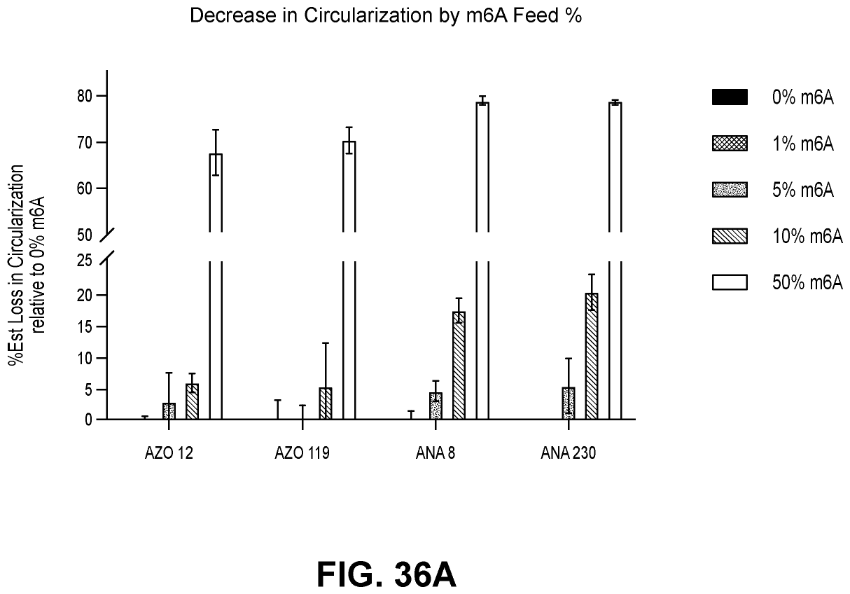

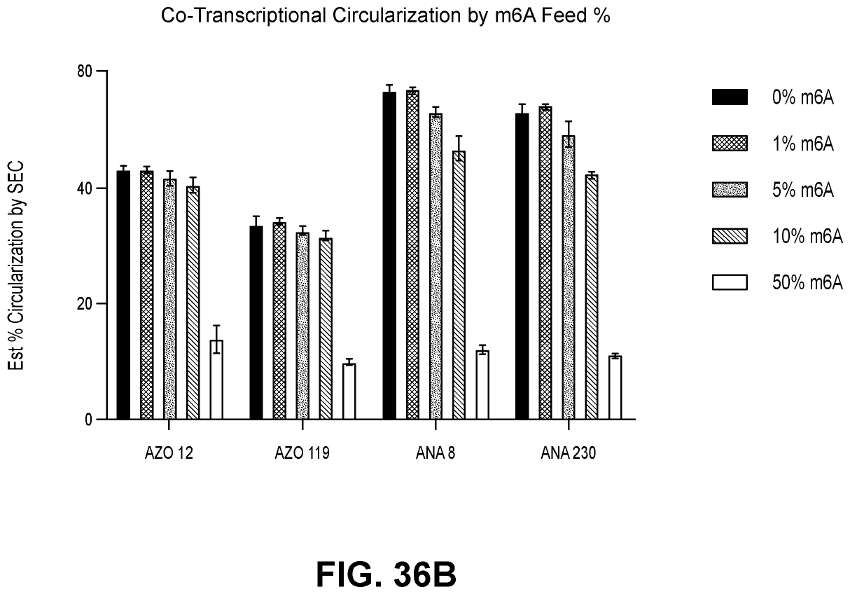

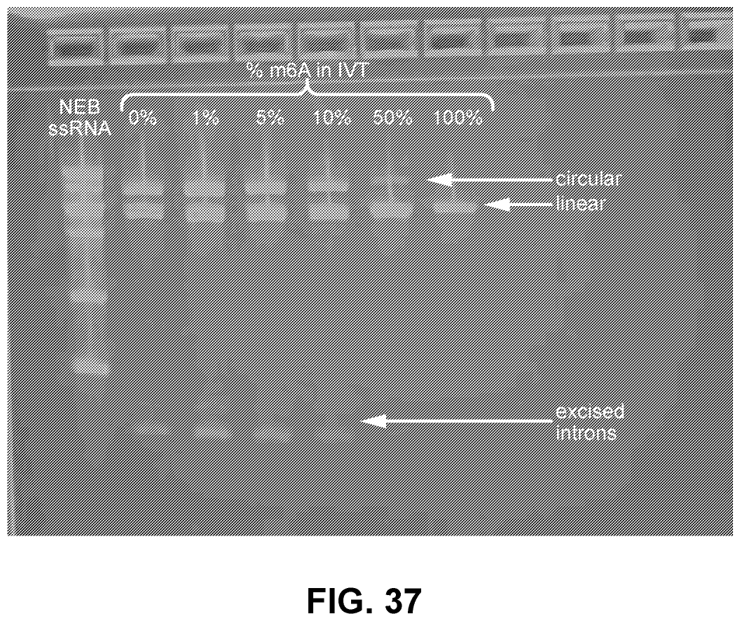

IVT reactions were conducted on four different DNA templates comprising: (1) a T7 polymerase promoter, (2) 5′ and 3′ permuted Anabaena or Azoarcus intron segments, (3) 5′ and 3′ exon segments, (4) a CVB3 TRES, (5) an expression sequence encoding firefly luciferase, and (6) a XbaI restriction site. The DNA templates comprised 5′ or 3′ intron segments developed from either an Anabaena intron position 8 or 230 permutation site in FIG. 8A or an Azoarcus intron position 12 or 119 permutation site in FIG. 8B. Each IVT reaction was given either 0%, 1%, 5%, 10%, 50% or optionally 100% m6A-TP. The resulting IVT products were purified using a silica column and analyzed by SEC-HPLC in 1× tris-EDTA running buffer (e.g., from Agilent) at a pH of 6 to determine splicing, estimated circularization, and estimated loss of circularization as compared to DNA templates that were not treated with m6A-TP. FIG. 36A depicts the calculated amount of circular RNA generated post IVT reaction with m6A-TP as compared to the autocatalytically generated circular RNA before enrichment for each IVT reaction performed. FIG. 36B depicts the estimated percent circularization for the DNA templates post IVT. The table below provides the nucleotide and/or nucleoside composition of the DNA templates. FIG. 37 provides an exemplary 2% electroporation gel of the DNA template comprising Anabaena introns permuted at position 230 (in FIG. 8A) alongside an NEB ssRNA ladder for reference. The electroporation gel was ran for 15 minutes before splicing activity was determined.

| | Intron | #A′s | % A's | #C′s | % C's | #G′s | % G's | #U′s | % U's |
|---|---|---|---|---|---|---|---|---|---|
| DNA | Anabaena | 757 | 27.09% | 758 | 27.13% | 721 | 25.81% | 558 | 19.97% |
| template 3 | 230 | | | | | | | | |
| DNA | Anabaena | | | | | | | | |
| template 4 | 8 | | | | | | | | |
| DNA | Azoarcus | 712 | 25.89% | 771 | 28.04% | 729 | 26.51% | 538 | 19.56% |
| template 5 | 12 | | | | | | | | |
| DNA | Azoarcus | | | | | | | | |
| template 6 | 119 | | | | | | | | |

### Example 12: Protecting Introns from Modifications

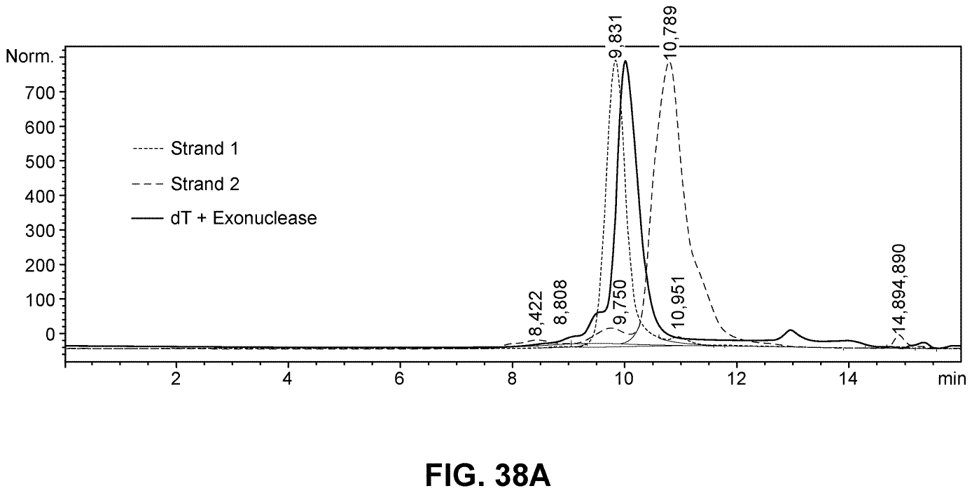

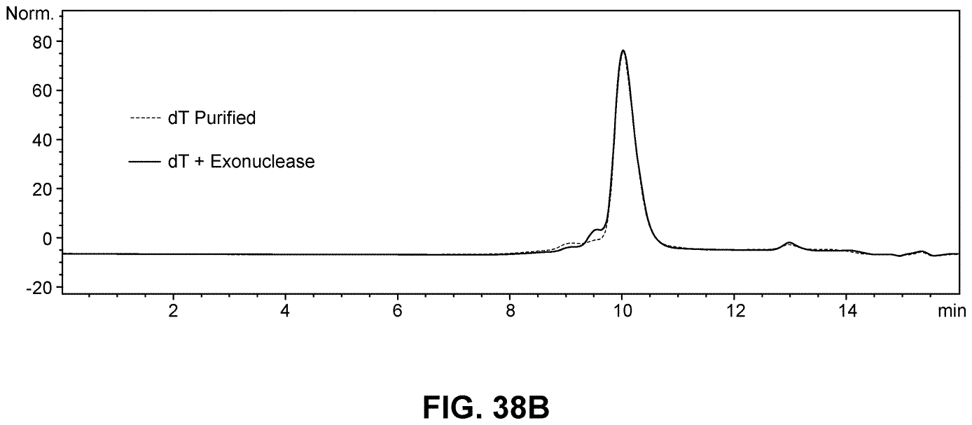

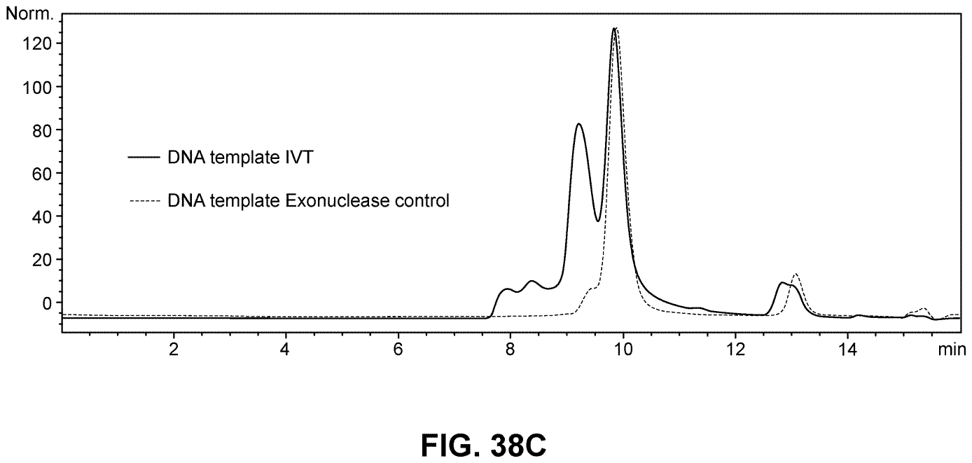

Two separate DNA templates were designed (e.g., Strand 1 and Strand 2). Strand 1 comprised a 3′ monotron sequence developed using the 3′ side of a Staphylococcus phage Twort intron permuted at position 16 in FIG. 29, a 5′ intron segment developed using a 5′ side of an Anabaena permuted at position 230 in FIG. 8A, and a Caprine kobuvirus TRES. Strand 2 comprised a 3′ intron segment developed using the 3′ side of an Anabaena intron permuted at position 230 in FIG. 8A and a 5′ intron segment developed using the 5′ side of a Staphylococcus phage Twort intron permuted at position 16 in FIG. 29. Both DNA templates further comprised: (1) a 5′ and 3′ exon segment, (2) an internal duplex region, (3) an internal spacer, and an expression sequence encoding part of a firefly luciferase protein. The expression sequences present in Strand 1 and Strand 2 combined are capable of encoding a fully functional firefly luciferase protein. Strand 1 and Strand 2 were synthesized in separate IVT reactions with magnesium concentrations of 7 mM. After synthesis of Strand 1 and Strand 2, both strands were purified using methods described herein. The purified IVT reaction products were analyzed by SEC-HPLC in 1× tris-EDTA and depicted as “Strand 1” and “Strand 2” in FIG. 38A. The purified strands were then incubated together in a solution comprising 3 mM of magnesium as well as 1 mM guanosine-monophosphate to facilitate trans-splicing between both strands and aid with circularization. The resulting circular RNA product were then isolated, purified using column and/or dT purification methods, treated with an endonuclease and analyzed by SEC-HPLC in 1× tris-EDTA (shown in FIG. 38A and FIG. 38B as “dT+Exonuclease”). FIG. 38B provides the resulting circular RNA product that did not receive exonuclease treatment but was oligo-dT purified. For comparison purposes, an IVT reaction was performed for a DNA template comprising: (1) a CVB3 IRES, (2) 5′ and 3′ intron segments developed from a permuted Anabaena intron at position 230 in FIG. 8A, (3) 5′ and 3′ Anabaena exon segments, and (4) an expression sequence encoding for firefly luciferase and further treated with an exonuclease control.

### Example 13: Assessing Immunostimulatory Effects

To deliver linear mRNA therapies, base modifications have been used to reduce the associated immunostimulatory effects. Absent base modification, linear mRNA impurities may trigger multiple immunostimulatory pathways (e.g., RIG-I, MDAS, TLR3, TLR7, TLR8) and may result in cytokine secretion and tolerability limitations.

Circular RNAs without base modifications were generated using a method comprising an in vitro transcription (IVT) reaction, during which circularization occurs co-transcriptionally, followed by purification processes. Purification reduces reactogenic species that may result in cytokine induction. Post-IVT, pre-purification SEC yields the presence of precursor and intron species. Post-IVT, post-purification SEC yields the removal of precursor and intron species.

J2 dsRNA ELISA was used to detect double stranded RNA impurities, the presence of which may be an indicator of RNA-induced reactogenicity. A549 IFN-β ELISA is a cell-based assay using a human A549 lung carcinoma cell line transfected with RNA via lipofectamine. Supernatants were collected after 24 hr incubation and evaluated for IFN-β response using an ELISA. A549s express pattern recognition receptors (PRRs), including RLRs and TLR2, TLR3, and TLR5.

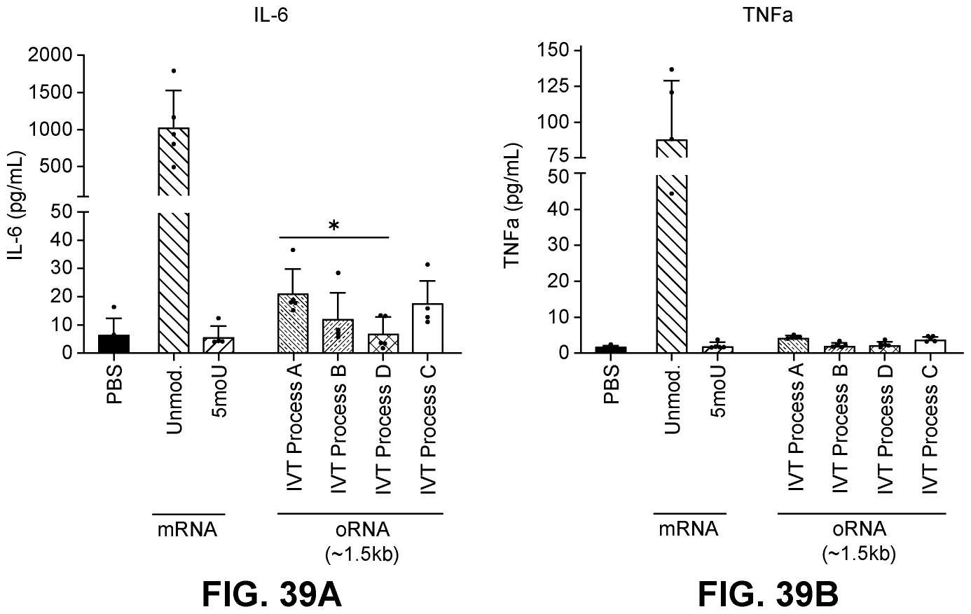

6-week old Balb/c mice were injected with a single 1.0 mg/kg i.v. dose of circular RNA generated using the disclosed methods or 5moU modified linear mRNA formulated in LNP. Blood serum was collected for a multiplex cytokine assay for proinflammatory cytokine profiles 6 hours post dose. The circular RNA showed reduced reactogenicity in mice using the IVT processes shown in FIG. 39A and FIG. 39B. The unmodified circular RNA showed comparable IL-6 and TNFα as compared to 5moU modified linear mRNA.

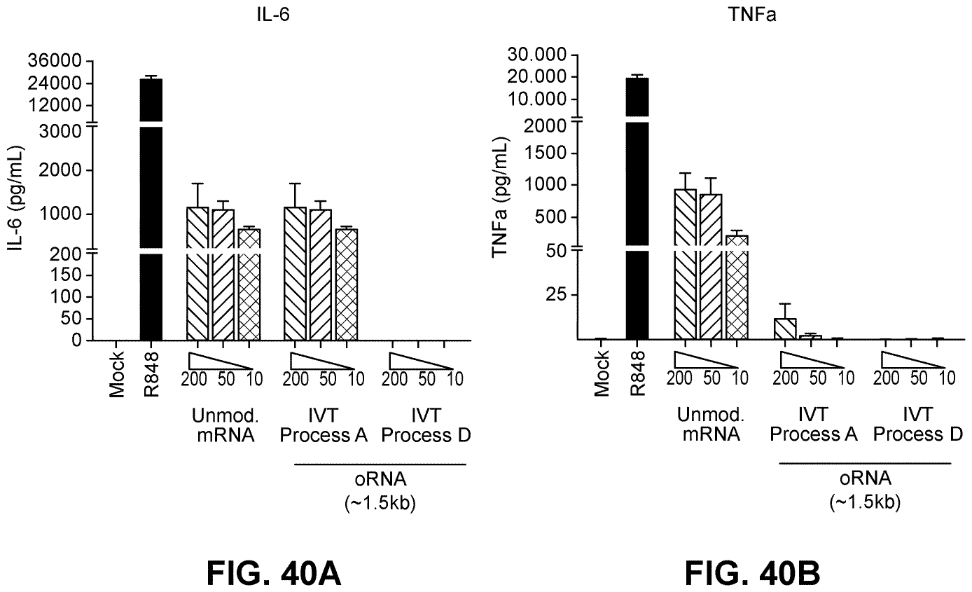

The human B-cell precursor leukemia cell line, BLaER1, can be transdifferentiated into monocytes by the addition of β-estradiol, IL-3, and M-CSF, and during this process, cells can lose CD19+ B-cell phenotype and gain CD11b+/CD14+ myeloid/monocyte phenotype. See, e.g., Rapino et al. “C/EBPA induces highly efficient macrophage transdifferentiation of B lymphoma and leukemia cell lines and impairs their tumorigenicity.” Cell Reports. 3, 4 (2013). Presence of ssRNA-detecting TLR7 and TLR8 were confirmed in transdifferentiated BLaER1 cells by the reactogenic response to the agonist R848, resulting in high levels of IL-6 and TNFα secretion. Circular RNAs generated using the disclosed methods were transfected into BLaER1 cells via lipofectamine. Supernatants were collected and assessed on a multiplexed cytokine assay 24 hr post transfection for proinflammatory cytokine response. A reduction of reactogenic response was observed from 200 ng to 10 ng of circular RNA transfected in Process A, and reactogenic impurities were undetectable in Process D (FIG. 40A and FIG. 40B).

### INCORPORATION BY REFERENCE

All publications, patents, and patent applications mentioned in this specification are herein incorporated by reference to the same extent as if each individual publication, patent, or patent application was specifically and individually indicated as being incorporated by reference herein.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

What is claimed is:

1. A circular RNA polynucleotide comprising, in the following order:
(a) a 3′ self-spliced exon segment, wherein the 3′ self-spliced exon segment comprises an exon segment and a 3′ nucleotide of a 3′ splice site dinucleotide;
(b) an intervening region; and
(c) a 5′ self-spliced exon segment, wherein the 5′ self-spliced exon segment comprises an exon and a 5′ nucleotide of a 5′ splice site dinucleotide.

2. The circular RNA polynucleotide of claim 1, wherein the 3′ nucleotide of the 3′ splice site dinucleotide is a Group I or Group II exon splice site dinucleotide, and wherein the 5′ nucleotide of the 5′ splice site dinucleotide is a Group I or Group II exon splice site dinucleotide.

3. The circular RNA polynucleotide of any one of claims 1-2, wherein the 3′ self-spliced exon segment and/or the 5′ self-spliced exon segment comprises a sequence having a percent sequence identity of about 70%, 75%, 80%, 85%, 90%, 91%, 92%, 93%, 94%, 95%, 96%, 97%, 98%, 99% or more to a sequence selected from SEQ ID NOs: 2990-3668, 25573, and 25574.

4. The circular RNA polynucleotide of any one of claims 1-3, wherein the circular RNA polynucleotide comprises at least one modified A, C, G, or U nucleotide or nucleoside.

5. The circular RNA polynucleotide of claim 4, wherein the modified nucleotide or nucleoside is:
a) one or more of m⁵U (5-methyluridine); m⁶A (N⁶-methyladenosine); s²U (2-thiouridine); Ψ (pseudouridine); Um (2′-O-methyluridine); m¹A (1-methyladenosine);
m²A (2-methyladenosine); Am (2′-O-methyladenosine); ms² m⁶A (2-methylthio-N⁶-methyladenosine); i⁶A (N⁶-isopentenyladenosine); ms²i6A (2-methylthio-N⁶ isopentenyladenosine); io⁶A (N⁶-(cis-hydroxyisopentenyl)adenosine); ms²io⁶A (2-methylthio-N⁶-(cis-hydroxyisopentenyl)adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms² hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m² ₂G (N²,N²-dimethylguanosine); m²Gm (N²,2′-O-dimethylguanosine); m² ₂Gm (N²,N²,2′-O-trimethylguanosine); Gr(p) (2′-O-ribosylguanosine(phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mCmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵S²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶ 2A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶ 2Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm ⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine); pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, N1-methylpseudouridine, and N2,N2-dimethyl-6-thio-guanosine; or
b) is selected from one or more of: 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine, 5-(2-amino)propyl uridine, 5-halocytidine, 5-halouridine, 4-acetylcytidine, 1-methyladenosine, 2-methyladenosine, 3-methyicytidine, 6-methyluridine, 2-methylguanosine, 7-methylguanosine, 2,2-dimethylguanosine, 5-methylaminoethyluridine, 5-methyloxyuridine, 7-deaza-adenosine, 6-azouridine, 6-azocytidine, 6-azothymidine, 5-methyl-2-thiouridine, 2-thiouridine, 4-thiouridine, 2-thiocytidine, dihydrouridine, pseudouridine, queuosine, archaeosine, naphthyl substituted naphthyl groups, an O- and N-alkylated purines and pyrimidines, N6-methyladenosine, 5-methylcarbonylmethyluridine, uridine 5-oxyacetic acid, pyridine-4-one, pyridine-2-one, aminophenol, 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, N1-methylpseudouridine, and alkylcarbonylalkylated nucleotides; or
c) is selected from one or more of: 5-methylcytidine, 5-methoxyuridine, 1-methyl-pseudouridine, N6-methyladenosine, and/or pseudouridine.

6. A circular RNA polynucleotide comprising the following elements arranged in the following order:
(a) at least a portion of a terminal element,
(b) a 3′ exon segment comprising a 3′ nucleotide of a 3′ splice site dinucleotide,
(c) an intervening region,
(d) a 5′ exon segment comprising a 5′ nucleotide of a 5′ splice site dinucleotide, and
(e) optionally, a portion of a monotron element;
wherein the 5′ and/or 3′ splice site dinucleotides are distinct from the natural splice site dinucleotide(s) associated with a natural Group I or Group II intron sequence.

7. The circular RNA polynucleotide of claim 6, wherein the monotron element sequence comprises a polynucleotide sequence that has a percent sequence identity of about 70%, 75%, 80%, 85%, 90%, 91%, 92%, 93%, 94%, 95%, 96%, 97%, 98%, 99% or more to a sequence selected from SEQ ID NOs: 2990-3187, 25573, and 25574.

8. The circular RNA polynucleotide of any one of claim 6 or 7, wherein element (d) comprises the first nucleotide of a 5′ Group I or Group II splice site dinucleotide and a natural exon sequence.

9. The circular RNA polynucleotide of claim 6-8, wherein element (b) comprises the second nucleotide of a 3′ Group I or Group II exon splice site dinucleotide and a natural exon sequence.

10. The circular RNA polynucleotide of any one of claims 1-8, comprising a 5′ internal duplex and a 3′ internal duplex.

11. The circular RNA polynucleotide of any one of claims 1-10, comprising a 5′ internal spacer and/or a 3′ internal spacer.

12. The circular RNA polynucleotide of any one of claims 1-11, wherein the intervening region comprises a coding sequence.

13. The circular RNA polynucleotide of any one of claims 1-11, wherein the intervening region comprises a noncoding sequence.

14. The circular RNA polynucleotide of one of claims 1-13, comprising a translation initiation element (TIE).

15. The circular RNA polynucleotide of claim 14, comprising, in the following order, a 3′ self-spliced exon segment, a translation initiation element (TIE), a coding sequence with which the TIE is not naturally associated, and a 5′ self-spliced exon segment.

16. The circular RNA polynucleotide of any one of claims 1-15, wherein the circular RNA polynucleotide is from about 50 nucleotides to about 15 kilobases in length.

17. The circular RNA polynucleotide of any one of claims 1-16, wherein the circular RNA polynucleotide:
a. has an in vivo duration of therapeutic effect in a subject of at least about 10 hours;
b. has a functional half-life of at least about 10 hours;
c. has a duration of therapeutic effect in a cell greater than or equal to that of an equivalent linear RNA polynucleotide comprising the same expression sequence;
d. has a functional half-life in a cell greater than or equal to that of an equivalent linear RNA polynucleotide comprising the same expression sequence;
e. has an in vivo duration of therapeutic effect in a subject greater than that of an equivalent linear RNA polynucleotide having the same expression sequence; and/or
f. has an in vivo functional half-life in a subject greater than that of an equivalent linear RNA polynucleotide having the same expression sequence.

18. A circular RNA polynucleotide comprising, in the following order, a 3′ self-spliced exon segment, an intervening region, and a 5′ self-spliced exon segment, wherein at least one self-spliced exon segment is selected from an exon segment comprising a sequence selected from SEQ ID NOs: 2990-3668, 25573, and 25574.

19. A precursor RNA polynucleotide useful for preparing the circular RNA polynucleotide of any one of claims 1-18.

20. A pharmaceutical composition comprising the circular RNA polynucleotide of any one of claims 1-18, the precursor RNA polynucleotide of claim 19, or combinations thereof.

21. The pharmaceutical composition of claim 20 and a transfer vehicle.

22. A method of treating a subject in need thereof, comprising administering to the subject the pharmaceutical composition of claim 20 or 21.
